SEQUENCE LISTING (6 sequences)

SEQ ID NO: 1 - protein, 1130 aa, homo sapiens [sequence omitted]

SEQ ID NO: 2 - protein, 27 aa, artificial
  His tagged leader sequence
MGYPYDVPDYAMAHHHHHHIEGRAAMD

SEQ ID NO: 3 - protein, 310 aa, homo sapiens
MGYPYDVPDYAMAHHHHHHIEGRAAMDPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQES

SEQ ID NO: 4 - protein, 273 aa, homo sapiens
VSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQES

SEQ ID NO: 5 - protein, 146 aa, homo sapiens
WEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVA

SEQ ID NO: 6 - protein, 98 aa, homo sapiens
FPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQ

(19) United States
(12) Patent Application Publication
(10) Pub. No.: US 2006/0030017 A1
(43) Pub. Date: Feb. 9, 2006

(54) Three-dimensional structure of c-Abl

(76) Inventors: Gabriele Fendrich, Basel (CH); Sandra Wendy Jacob, Rantzwiller (FR); Anthony Edward Klon, Lawrenceville, NJ (US); Paul William Manley, Arlesheim (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/086,482

(22) Filed: Mar. 22, 2005

Related U.S. Application Data
(60) Provisional application No. 60/558,457, filed on Apr. 1, 2004.

(51) Int. Cl. C12N 9/12; G01N 33/48; G06F 19/00

(52) U.S. Cl. 435/194; 702/19

(57) ABSTRACT

The present invention concerns c-Abl, in particular, the three-dimensional structure of c-Abl containing an active DFG conformation. The atomic coordinates of c-Abl possessing this active DFG motif complexed with a specific ligand are also provided.

### FIELD OF THE INVENTION

The present invention relates to a recombinant form of c-Abl, in particular, the three-dimensional structure of this protein in a catalytically active conformation. The invention further relates to the use of the three-dimensional structure of c-Abl disclosed herein for identifying and designing ligands or low molecular weight compounds which inhibit the tyrosine kinase activity of c-Abl.

### BACKGROUND OF THE INVENTION

Human c-Abl is a structurally complex 145 kDa protein possessing three SRC homology domains (SH1-SH3), located towards the amino terminus. The tyrosine kinase activity of c-Abl is normally tightly regulated by the phosphorylation/de-phosphorylation of residues Tyr245, Tyr412 and Ser465 (isoform B) or Tyr226, Tyr393 and Ser446 (isoform A), together with a number of inter- and intra-molecular interactions. A recent structure of c-Abl in the assembled inactive state shows that critical interactions between the SH2 domain and the kinase domain depend upon a conformational change induced by the binding of myristate within the C-terminal lobe of the kinase domain. See Nagar et al., Cell, Vol. 112, pp. 859-871 (2003).

Kinases can adopt distinct conformations, in which there are large variations in the size and shape of the ATP pocket. See Huse and Kuriyan, Cell, Vol. 109, pp. 275-282 (2002). The crystal structure of STI571 (GLEEVEC) in complex with Abl kinase reveals that the drug targets an inactive conformation of the kinase. In addition to occupying the adenosine sub-site of the ATP pocket, and due to the small size of Thr315, the inhibitor is able to reach past this residue which contributes to the shape of the hydrophobic pocket used to design selectivity into kinase inhibitors, and occupy the space left by the conformational change of the DFG motif. This change is required for the tight binding of STI571. A negative aspect of this type of binding is that inhibitors, such as STI571 are exposed to residues that are neither involved in binding ATP, nor necessary for the function of the enzyme. These residues can be mutated to give changes in structure that prevent STI571 binding without losing enzyme activity, leading to resistance. Thus, in the case of the treatment of advanced chronic myelogenous leukemia (accelerated or blast crisis phase) or of acute lymphoblastic leukemia, patients frequently develop resistance to STI571 therapy and relapse after initially responding to treatment. In the majority of relapsed patients, resistance is the result of point mutations in the Abl kinase domain of the Bcr-Abl protein. These mutant forms of Bcr-Abl are desensitized towards inhibition by STI571, allowing clonal expansion of Ph+cells resistant to the drug. Following the initial findings of Gorre et al. who first isolated the Thr315Ile mutant [see Gorre et al., Science, Vol. 293, pp. 876-880 (2001), studies in which the Bcr-Abl kinase domains from STI571-resistant patients were sequenced revealed more than 30 distinct mutations. Cells are able to generate these mutant forms of Bcr-Abl, since the point mutations in the Abl tyrosine kinase domain do not impede the binding of ATP to the catalytically active conformation of the enzyme, nor hinder the catalytic activity of the active conformation. However, in the case of a drug molecule which binds to the catalytically active ATP-binding conformation to inhibit the kinase, there would be far less scope for drug-resistant, functionally competent mutant forms of the enzyme to undergo clonal expansion, leading to disease progression.

The crystal structure of a complex between PD173955, an inhibitor of Abl kinase, and the kinase domain of Abl has been published and reveals that the ligand binds within the adenine-binding site via two H-bonds and a range of van der Waals interactions. See Nagar et al., Cancer Res, Vol. 62, pp. 4236-4243 (2002). Although this complex has a structure that resembles the catalytically active conformation of other kinases, the DFG motif is in an inactive conformation unsuitable for optimal binding of ATP. See Cowan-Jacob et al., Mini Rev Med Chem, Vol. 4, pp. 285-299 (2004).

It is therefore clear that additional information regarding the conformation of active and inactive forms of c-Abl is necessary to gain a better understanding of this tyrosine kinase and to find ways to inhibit its function when associated with debilitating disease states. To that end, the present invention describes for the first time to our knowledge the three dimensional structure of recombinant human c-Abl possessing a DFG motif in an active conformation determined by crystallization of c-Abl and it is demonstrated herein that this structural information may be used for identifying and/or designing inhibitors of c-Abl with therapeutic usefulness.

### SUMMARY OF THE INVENTION

The present invention provides the three-dimensional structure of c-Abl comprising a DFG motif in an active conformation thereby enabling the identification and design of ligands or low molecular weight molecules which may bind to c-Abl, particularly inhibitors of c-Abl.

In one aspect of the invention, a crystal of c-Abl comprising a unit cell with dimensions of a=182.6 Å, b=58.3 Å, c=104.0 Å, beta=119.2 o, with space group C2 is provided.

In another aspect of the invention, a three-dimensional structure of c-Abl comprising the atomic structure coordinates of Table 6 is provided.

In yet another aspect, a crystal of c-Abl comprising an active conformation of the DFG motif is provided.

In a further aspect of the invention, a crystal of c-Abl in which at least one ligand or low molecular weight compound is bound to c-Abl is provided.

In yet another aspect of the invention, a computer readable medium comprising data storage material encoded with computer readable data wherein said data comprises the atomic coordinates of c-Abl as disclosed in Table 6 is provided.

In another aspect, the invention provides a method of determining the three-dimensional structure of c-Abl comprising:
(a) crystallisation of c-Abl, the amino acid sequence of which comprises SEQ ID NO. 4, or a mutant, fragment or homologue thereof; and
(b) utilizing the atomic coordinates disclosed in Table 6 in whole or in part to determine the three-dimensional structure of said polypeptide.

In a further aspect, the invention provides a method of determining the three-dimensional structure of a complex comprising c-Abl, the amino acid sequence of which comprises SEQ ID NO. 4, or a mutant, fragment or homologue thereof, bound to at least one ligand comprising:
(a) obtaining x-ray diffraction data for crystals of the complex; and
(b) utilizing the atomic coordinates disclosed in Table 6 in whole or in part to define the three-dimensional structure of the complex.

In another aspect, the invention provides a method of identifying a ligand or low molecular weight compound that binds to c-Abl comprising:
(a) utilizing the three dimensional structure of c-Abl derived in whole or in part from the set of atomic coordinates in Table 6 to identify a potential ligand or low molecular weight compound that binds to c-Abl; and
(b) selecting those ligands or low molecular weight compounds that bind to c-Abl.

In another aspect of the invention, a method of identifying a ligand or low molecular weight compound that selectively binds to c-Abl is provided comprising:
(a) utilizing the three dimensional structure of c-Abl derived in whole or in part from the set of atomic coordinates in Table 6 to identify a potential ligand or low molecular weight compound that binds selectively to c-Abl and to no other tyrosine kinase; and
(b) selecting only those ligands or low molecular weight compounds which bind selectively to c-Abl and to no other tyrosine kinase.

In another aspect of the invention, a method of designing a ligand or low molecular weight compound capable of binding to c-Abl is provided comprising:
(a) using the atomic coordinates of Table 6 in whole or in part to determine the three dimensional structure of c-Abl;
(b) screening said c-Abl with candidate ligands or low molecular weight compounds to determine which bind to c-Abl;
(c) selecting those ligands or low molecular weight compounds which bind to c-Abl; and
(d) modifying those ligands or low molecular weight compounds which bind to maximize physical properties, such as solubility, affinity, specificity or potency.

In another aspect of the invention, a method of designing a ligand or low molecular weight compound capable of binding selectively to c-Abl is provided comprising:
(a) using the atomic coordinates disclosed in Table 6 in whole or in part to determine the three dimensional structure of c-Abl;
(b) screening said c-Abl with candidate ligands or low molecular weight compounds to determine which selectively bind to c-Abl and;
(c) selecting those ligands or low molecular weight compounds which selectively bind to c-Abl; and
(d) modifying those ligands or low molecular weight compounds which bind to maximize physical properties, such as solubility, affinity, specificity or potency.

In preferred embodiments of this invention, the candidate ligands or low molecular weight compounds are screened and designed in silico.

### DETAILED DESCRIPTION OF THE INVENTION

The term “unit cell”, according to the invention, refers to the basic shape block. The entire volume of a crystal is constructed by regular assembly of such blocks. Each unit cell comprises a complete representation of the unit of pattern, the repetition of which builds up the crystal.

The term “space group”, according to the invention, refers to the arrangement of symmetry elements of a crystal.

The term “ligand”, according to the invention, refers to a molecule or group of molecules that bind to one or more specific sites of c-Abl. Preferred are ligands that selectively bind to the active site of c-Abl. Ligands according to the invention are preferably low molecular weight molecules.

The term “low molecular weight compound” or “low molecular weight molecules”, according to the invention, refers to preferably organic compounds generally having a molecular weight less than about 1000, more preferably less than about 600. Most preferably, said low molecular weight compounds or ligands selectively inhibit the biological activity of c-Abl.

The term “selective binding” refers to the ability of a ligand or low molecular weight compound to bind to c-Abl but not to any other tyrosine kinase.

The term “in silico” or an “in silico screen”, as used herein, refers to computational or computer methodologies and modeling to predict, e.g., protein function or protein-protein, protein-ligand interaction.

The “DFG motif”, as referred to herein, relates to a stretch of amino acids on the c-Abl protein, e.g., the human form of c-Abl as disclosed in Swiss Prot Accession No. P00519, Isoform 1A) (SEQ ID NO. 1) from amino acids 381-383 that is highly-conserved among protein kinases. The aspartate group (D) from this motif is necessary for the function of the protein because it is involved in positioning magnesium ions and the phosphate groups of ATP correctly for catalysis. An “active DFG motif conformation” refers to the state in which the Phe residue of the motif is buried in a hydrophobic pocket in the groove between the N-terminal, e.g., residues Met290 and Ile293; and C-terminal lobes, e.g., residues His362 and Ala380, of the kinase. See Yamaguchi and Hendrickson, Nature, Vol. 384, pp. 484-489 (1996). This is in contrast to an inactive conformation where the Phe residue, and or other residues, such as the Asp are flipped out in the case of Phe382 [see Schindler et al., Science, Vol. 289, pp. 1938-1942 (2000)] or in the case of Asp [see Nagar et al. (2002), supra] and are therefore not correctly positioned for catalysis. An “optimum active DFG conformation” refers to an active DFG motif conformation with all residues, including those of this motif and the neighboring residues, e.g., Lys271, Glu286, Asp381, Asp363 and Asn368, in a conformation suitable for catalysis.

The terms “active” and “inactive” with regard to the conformation of the c-Abl protein refer to the conformation of the protein which has the DFG motif in the correct conformation for catalytic activity, the activation loop in an extended conformation such that main chain atoms do not traverse the substrate binding site, and Lys-271 and Glu-286 close enough within experimental error to form a salt bridge in the active site (“active”), whereas the inactive conformation can have any one or more of these components in a different conformation that is not suitable for activity (“inactive”). A “catalytically active conformation” refers to a conformational state of the c-Abl protein that is theoretically suitable for performing the kinase reaction based on similarity to structures of other kinases in the active state, e.g., the LCK kinase structure. See Yamaguchi and Hendrickson (1996), supra.

The term “activation loop” refers to a stretch of amino acid residues that starts from the DFG motif and finishes at residue Phe-401 in Abl kinase (SEQ ID NO. 1). In tyrosine kinases this region usually contains one or more tyrosines (Tyr-393 in Abl), that is or are phosphorylated for the kinase to achieve full catalytic activity.

While the sequence of the wild type form of human c-Abl as provided in Swiss Prot Accession No. P00519, Isoform 1A is utilized herein, it is contemplated that other forms and variants of c-Abl may be employed in the instant invention by one of skill in the art without undue experimentation.

In context of a c-Abl inhibitor, the terms “peptide” or “peptide derivative” are intended to embrace a “peptidomimetic” or “peptide analogue” which complement the three-dimensional structure of the binding site of c-Abl or can be designed with improved physical or chemical properties to bind with the three-dimensional binding site of the c-Abl as provided in the present invention.

The term “mutant” refers to possible differences within the wild-type sequence of c-Abl and/or the kinase domain sequence set forth in SEQ ID NO. 4 by deletion, insertion, extension or replacement of one or more selected amino acids.

According to the present invention, the term “mutant” also refers to a polypeptide, whose amino acid sequence differs from the wild type sequence or the kinase domain sequence given in SEQ ID NO.4 by deletion, insertion, extension or preferably replacement of one or more selected amino acids. For example, a c-Abl mutant of the present invention is preferably at least 50% homologous to the wild type sequence, more preferably at least 80% homologous to the wild type sequence, most preferably at least 90% homologous to the wild type sequence.

An “N-terminal extension” of c-Abl, according to the invention, comprises the addition of amino acids at the N-terminus of the full-length c-Abl or a fragment thereof. For example, such N-terminal extension may include the addition of amino acids comprising a stretch of histidine residues that may be utilized to facilitate purification of the protein and which is later cleaved from the protein sequence.

c-Abl may be crystallizable with or without at least one ligand. According to the present invention, c-Abl crystals are stable if kept under suitable conditions. For example, the crystals are stable in their mother liquor at 4° C. for at least 3-4 weeks. Preferably, the crystals are cryo-cooled and stored in liquid nitrogen.

According to the invention, c-Abl may be prepared by methods well-known to one of skill in the art including, but not limited to, isolation from natural sources, e.g., cultured human cells or preferably by recombinant heterologous expression. Expression of recombinant c-Abl is achievable in eukaryotic or prokaryotic systems. For example, recombinant human c-Abl may be expressed in bacteria or Sf9 cells, e.g., as a Strep-tag fusion protein, a glutathione-S-transferase (GST) fusion protein, a histidine-tagged fusion protein or as an untagged protein. If desired, the fusion partner is removed before crystallization. The heterologously produced c-Abl to be used for crystallization is potentially biologically active. Such activity may be determined by morphological, biochemical or viability analysis well-known in the art.

Methods for the preparation of c-Abl mutants are commonly-known in the art. For example, c-Abl mutants may be prepared by expression of c-Abl DNA previously modified in its coding region by oligo-nucleotide directed mutagenesis.

In the present invention, purified c-Abl is preferably at least 90% homogeneous. Protein homogeneity is determinable according to analytical methods well-known in the art, e.g., sequence analysis, electrophoresis, spectroscopic or chromatographic techniques. The purified protein is potentially enzymatically active. Appropriate assays for determining c-Abl activity towards a suitable substrate, e.g., a natural substrate or a synthetic substrate, are known in the art. In one embodiment of the invention, at any step prior to crystallization c-Abl may be complexed with a low molecular weight compound or ligand which is capable of suitably binding to c-Abl. Preferred is a compound inhibiting c-Abl activity. Kinase inhibition is determinable employing assays known in the art. Suitable inhibitors include kinase inhibitors which act on the catalytic site to inhibit c-Abl activity.

Various methods of crystallization familiar to one of skill in the art can be used in the claimed invention including vapor diffusion, dialysis or batch crystallization. In vapor diffusion crystallization, a small volume, i.e., a few microliters, of protein solution is mixed with a solution containing a precipitant. This mixed volume is suspended over a well containing a small amount, i.e., about 0.15-1 mL, of precipitant. Vapor diffusion between the drop and the well will result in crystal formation in the drop.

The dialysis method of crystallization utilizes a semipermeable, size-exclusion membrane that retains the protein but allows small molecules, i.e., buffers and precipitants, to diffuse in and out. In dialysis, rather than concentrating the protein and the precipitant by evaporation, the precipitant is allowed to slowly diffuse through the membrane and reduce the solubility of the protein while keeping the protein concentration fixed.

The batch method generally involves the slow addition of a precipitant to an aqueous solution of protein until the solution just becomes turbid, at this point the container can be sealed and left undisturbed for a period of time until crystallization occurs. In the batch technique the precipitant and the target molecule solution are simply mixed. Supersaturation is achieved directly rather than by diffusion. Often the batch technique is performed under oil. The oil prevents evaporation and extremely small drops can be used. For this, the term “microbatch” is used. A modification of this technique is not to use paraffin oil (which prevents evaporation completely) but rather use silicone oil or a mixture of silicone and paraffin oils so that a slow evaporation is possible.

The claimed invention can encompass any and all methods of crystallization. One skilled in the art can choose any of such methods and vary the parameters such that the chosen method results in the desired crystals. Crystallization methods are also described in Crystallization of Biological Macromolecules, McPherson, Ed., Cold Spring Harbor Laboratory Press, NY (1999)—ISBN 0-87969-527-7, which is hereby incorporated by reference in its entirety.

One preferred method of crystallization of c-Abl involves mixing a c-Abl solution with a “reservoir buffer”, resulting in a lower concentration of the precipitating agent than necessary for crystal formation. For crystal formation, the concentration of the precipitating agent has to be increased, e.g., by addition of precipitating agent, e.g., by titration, or by allowing the concentration of precipitating agent to balance by diffusion between the crystallization buffer and a reservoir buffer. Under suitable conditions such diffusion of water or volatile precipitating agent occurs along the gradient of precipitating agent, e.g., between the reservoir buffer having a higher concentration of precipitating agent and the crystallization buffer having a lower concentration of precipitating agent. Diffusion may be achieved, e.g., by vapor diffusion techniques allowing diffusion of water in the common gas phase. Known techniques are, e.g., vapor diffusion methods, such as the “hanging drop” or the “sitting drop” method. In the vapor diffusion method a drop of crystallization buffer containing the protein is hanging above or sitting beside a much larger pool of reservoir buffer. Alternatively, the equilibration of the precipitating agent can be achieved through a semi-permeable membrane (dialysis method) that separates the crystallization buffer from the reservoir buffer and prevents dilution of the protein into the reservoir buffer.

Formation of c-Abl crystals can be achieved under various conditions, including the conditions disclosed herein, which are essentially determined based on the following parameters: pH, presence of salts and additives, precipitating agent, protein concentration and temperature. The pH may range, e.g., from about 4.0-10.0.

The present invention also relates to a computer readable medium having stored a model of the c-Abl crystal structure. In a preferred embodiment, said model is built from all or part of X-ray diffraction data using methodologies familiar to one of skill in the art. The atomic coordinates are shown in Table 6 appended hereto.

The present invention provides the structure coordinates of recombinant human c-Abl. The term “structure coordinates” or “atomic coordinates” refers to mathematical coordinates derived from the mathematical equations (Fourier transformation) related to the diffraction pattern obtained on a monochromatic beam of X-rays by the atoms (scattering centers) of a crystal comprising a c-Abl. The diffraction data are used to calculate an electron density map of the repeating unit of the crystal. The electron density maps are used to establish the positions of the individual atoms within the unit cell of the crystal.

Structural coordinates of a crystalline composition of this invention may be stored in a machine-readable form on a machine-readable storage medium, e.g., a computer hard drive, diskette, DAT tape, CD, DVD, etc., for display as a three-dimensional shape or for other uses involving computer-assisted manipulation of, or computation based on, the structural coordinates or the three-dimensional structures they define. For example, data defining the three dimensional structure of a protein of c-Abl, or portions or structurally similar homologues of such proteins, may be stored in a machine-readable storage medium, and may be displayed as a graphical three-dimensional representation of the protein structure, typically using a computer capable of reading the data from said storage medium and programmed with instructions for creating the representation from such data.

According to the present invention, a three-dimensional c-Abl model is obtainable from a crystal of c-Abl polypeptide, the amino acid sequence of which comprises SEQ ID NO. 4, or a mutant, fragment or homologue thereof. Such a model can be built or refined from all or part of the c-Abl structure data of the present invention using the X-ray diffraction coordinates, particularly the atomic structure coordinates of Table 6. Ideally, said models contemplated herein all possess a DFG motif in the active conformation.

The knowledge obtained from the three-dimensional model of c-Abl can be used in various ways. It can be used to identify chemical entities, e.g., small organic and bioorganic molecules, such as peptidomimetics and synthetic organic molecules that bind to c-Abl and preferably block or prevent a c-Abl mediated or associated process or event. Using the three-dimensional structure of c-Abl, the skilled artisan can construct a model of c-Abl. For example, every atom can be depicted as a sphere of the appropriate van der Waals radius, and a detailed surface map of c-Abl can be constructed.

Chemical entities that have a surface that mimics the accessible surface of the catalytic binding site of c-Abl can be constructed by those skilled in the art. By way of example, the skilled artisan can screen three-dimensional structural databases of compounds to identify those compounds that position appropriate functional groups in similar three-dimensional structural arrangement, then build combinatorial chemistry libraries around such chemical entities to identify those with high affinity to the catalytic binding site of c-Abl.

Ligands or small molecular compounds can be identified from screening compound databases or libraries and using a computational means to form a fitting operation to a binding site on c-Abl. The three-dimensional structure of c-Abl as provided in the present invention in whole or in part by the structural coordinates of Table 6, can be used together with various docking programs.

The potential inhibitory or binding effect of a chemical entity on c-Abl may be analyzed prior to its actual synthesis and testing by the use of computer-modeling, or “in silico”, techniques familiar to one of skill in the art. If the theoretical structure of the given chemical entity suggests insufficient interaction and association between it and c-Abl, the need for synthesis and testing of the chemical entity is obviated. However, if computer modeling indicates a strong interaction, the molecule may then be synthesized and tested for its ability to bind to c-Abl. Thus, expensive and time-consuming synthesis of inoperative compounds may be avoided.

An inhibitory or other binding compound of c-Abl may be computationally evaluated and designed by means of a series of steps in which chemical entities or fragments are screened and selected for their ability to associate with the individual binding sites of c-Abl. Thus, one skilled in the art may use one of several methods to screen chemical entities or fragments for their ability to associate with c-Abl. This process may begin by visual inspection of, e.g., the binding site on a computer screen based on the structural coordinates of Table 6 in whole or in part. Selected fragments or chemical entities may then be positioned in a variety of orientations, or “docked,” within the catalytic binding site of c-Abl. Docking may be accomplished using software, such as Quanta and SYBYL, followed by energy minimization and molecular dynamics with standard molecular mechanics force fields, such as CHARMM or AMBER. Specialized computer programs may be of use for selecting interesting fragments or chemical entities. These programs include, e.g., GRID, available from Oxford University, Oxford, UK; 5 MCSS or CATALYST, available from Molecular Simulations, Burlington, Mass.; AUTODOCK, available from Scripps Research Institute, La Jolla, Calif.; DOCK, available from University of California, San Francisco, Calif.; and XSITE, available from University College of London, UK.

Using molecular replacement to exploit a set of coordinates, such as those of Table 6 of the invention, the structure of a crystalline c-Abl or portion thereof can, e.g., be bound to one or more ligands or low molecular weight compounds to form a complex.

The term “molecular replacement” refers to a method that involves generating a preliminary structural model of a crystal whose structural coordinates are unknown, by orienting and positioning a molecule whose structural coordinates are known, e.g., the c-Abl coordinates within the unit cell of the unknown crystal, so as to best account for the observed diffraction pattern of the unknown crystal. Phases can then be calculated from this model, and combined with the observed amplitudes to give an approximated Fourier synthesis of the structure whose coordinates are unknown. This in turn can be subject to any of the several forms of refinement to provide a final accurate structure. Using the structural coordinates provided by this invention, molecular replacement may be used to determine the structural coordinates of a crystalline co complex, unknown ligand, mutant or homolog, or of a different crystalline form of c-Abl. Additionally, the claimed crystal and its coordinates may be used to determine the structural coordinates of a chemical entity that associates with c-Abl.

“Homology modeling” according to the invention involves constructing a model of an unknown structure using structural coordinates of one or more related proteins, protein domains and/or one subdomain. Homology modeling may be conducted by fitting common or homologous portions of the protein or peptide whose three dimensional structure is to be solved to the three-dimensional structure of homologous structural elements. Homology modeling can include rebuilding part or all of a three-dimensional structure by replacement of amino acids or other components by those of the related structure to be solved.

Molecular replacement according to the present invention uses a molecule having a known structure. The three-dimensional structure of c-Abl provided in whole or in part in Table 6 in a machine-readable form on a data-carrier can be used as a starting point to model the structure of an unknown crystalline sample. This technique is based on the principle that two molecules which have similar structures, orientations and positions in the unit cell diffract similarly. Molecular replacement involves positioning the known structure in the unit cell in the same location and orientation as the unknown structure. Once positioned, the atoms of the known structure in the unit cell are used to calculate the structure factors that would result from a hypothetical diffraction experiment. This involves rotating the known structure in the six dimensions (three angular and three spatial dimensions) until alignment of the known structure with the experimental data is achieved. This approximate structure can be fine-tuned to yield a more accurate and often higher resolution structure using various refinement techniques. For instance, the resultant model for the structure defined by the experimental data may be subjected to rigid body refinement in which the model is subjected to limited additional rotation in the six dimensions yielding positioning shifts of under about 5%. The refined model may then be further refined using other known refinement methods. The present invention also enables homologues and mutants of c-Abl and the solving of their crystal structure. Based on the three-dimensional structure of c-Abl as provided in the present invention and using the atomic coordinates of Table 6 in whole or in part, the effects of site-specific mutations can be predicted. More specifically, the structural information provided herein permits the identification of desirable sites for amino acid modification, particularly amino acid mutation resulting in substitutional, insertional or deletional variants. Such variants may be designed to have special properties, particularly properties distinct from wild-type c-Abl, such as altered catalytic activity. Substitutions, deletions and insertions may be combined to arrive at a desired variant. Such variants can be prepared by methods well-known in the art, e.g. starting from wild-type c-Abl or by de novo synthesis.

c-Abl may also crystallize in a form different from the one disclosed herein. The structural information provided, for example, in the wild type sequence (Swiss Prot Accession No. P00519, Isoform A) (SEQ. ID NO. 1), SEQ ID NO. 4 and Table 6 in whole or in part, is also useful for solving the structure of other crystal forms. Furthermore, it may serve to solve the structure of a c-Abl mutant, a c-Abl co-complex or a sufficiently homologous protein.

The c-Abl structural information provided herein is useful for the design of ligands or small molecule compounds which are capable of selectively interacting with c-Abl and thereby specifically modulating the biological activity of c-Abl. In particular, the coordinates disclosed herein reflect catalytically active c-Abl possessing an active DFG motif conformation suitable for optimum binding of ATP. Furthermore, this information can be used to design and prepare c-Abl mutants, e.g., mutants with altered catalytic activity, model the three-dimensional structure and solve the crystal structure of proteins, such as c-Abl homologues, c-Abl mutants or c-Abl co-complexes, involving, e.g., molecular replacement.

Preferred is a method for designing a c-Abl inhibitor which interacts at the active binding site possessing the optimum active DFG conformation. The present invention may also be used to design ligands or low molecular weight compounds which specifically inhibit c-Abl and which specifically do not bind to other tyrosine kinases.

One approach enabled by this invention is the use of the structural coordinates of c-Abl to design chemical entities that bind to or associate with c-Abl and alter the physical properties of the chemical entities in different ways. Thus, properties such as, e.g., solubility, affinity, specificity, potency, on/off rates or other binding characteristics may all be altered and/or maximized. One may design desired chemical entities by probing a c-Abl crystal comprising c-Abl with a library of different entities to determine optimal sites for interaction between candidate chemical entities and c-Abl. For example, high-resolution X-ray diffraction data collected from crystals saturated with solutes allows the determination of where each type of solute molecule adheres. Small molecules that bind tightly to those sites can then be designed and synthesized and tested for the desired activity. Once the desired activity is obtained, the molecule can be further altered to maximize desired properties.

The invention also contemplates computational screening of small-molecule databases or designing of chemical entities that can bind in whole or in part to c-Abl. They may also be used to solve the crystal structure of mutants, co-complexes or the crystalline form of any other molecule homologous to, or capable of associating with, at least a portion of c-Abl. One method that may be employed for this purpose is molecular replacement. An unknown crystal structure, which may be any unknown structure, such as, e.g., another crystal form of c-Abl, a c-Abl mutant or peptide or a co-complex with c-Abl, or any other unknown crystal of a chemical entity that associates with c-Abl that is of interest, may be determined using the whole of part of the structural coordinates set forth in Table 6. This method provides an accurate structural form for the unknown crystal far more quickly and efficiently than attempting to determine such information without the invention herein.

In one preferred embodiment of the invention, candidate ligands are screened in silico. The information obtained can be used to obtain specific inhibitors of c-Abl. For example, an inhibitor which inhibits only c-Abl but not other tyrosine kinases.

In another preferred embodiment of the invention, a method is provided to design ligands which inhibit the activity of c-Abl. In a preferred embodiment, a method is provided to design a selective inhibitor which is specific for only c-Abl and not for other tyrosine kinases.

The design of chemical entities that inhibit c-Abl generally involves consideration of at least two factors. First, the chemical entity must be capable of physically or structurally associating with c-Abl, preferably at the catalytic site of c-Abl. The association may be any physical, structural or chemical association, such as, e.g., covalent or non-covalent binding, or van der Waals, hydrophobic or electrostatic interactions. Second, the chemical entity must be able to assume a conformation that allows it to associate with c-Abl, preferentially at the catalytic site of c-Abl possessing the DFG motif for optimum ATP binding as reflected in the coordinates for c-Abl disclosed herein. Although not all portions of the chemical entity will necessarily participate in the association with c-Abl, those non-participating portions may still influence the overall conformation of the molecule. This in turn may have a significant impact on the desirability of the chemical entity. Such conformational requirements include the overall three-dimensional structure and orientation of the chemical entity in relation to all or a portion of the binding site.

Once a compound has been designed or selected by the above methods, the efficiency with which that compound may bind to c-Abl may be tested and modified for the maximum desired characteristic(s) using computational or experimental evaluation. Various parameters can be maximized depending on the desired result. These include, but are not limited to, specificity, affinity, on/off rates, hydrophobicity, solubility and other characteristics readily identifiable by the skilled artisan.

The present invention also relates to identification of compounds which inhibit c-Abl activity. In particular, said compounds are useful in preventing or treating disorders mediated by c-Abl, or variants thereof, e.g., proliferative diseases, such as CML and GIST in which an aberrant BCR-ABL hybrid is implicated, as well as other nonproliferative diseases familiar to one of skill in the art, including but not limited to, e.g., those in which c-Abl is implicated in the trafficking of pathogens, such as prions or viral proteins, into cell nuclei.

For the above uses, the required dosage will depend on the mode of administration, the particular condition to be treated and the desired effect. In general, satisfactory results are to be obtained systematically at daily dosages from about 0.1 mg/kg to about 100 mg/kg body weight. A daily dosage in a larger mammal, e.g., human, is in the range from about 0.5 mg to about 2,000 mg, conveniently administered, e.g., in divided doses up to 4 times a day or in delayed dosage form. The compounds may be administered by any conventional route, in particular, enterally, e.g., orally, e.g., in the form or tablets or capsules, or parenterally, e.g., in the form of injectable solutions or suspensions; topically, e.g., in the form of lotions, gels, ointments or creams; or in a nasal or a suppository form. Pharmaceutical compositions comprising said compound in free form or in a pharmaceutically acceptable salt form in association with at least one pharmaceutically acceptable carrier or diluent may be manufactured in conventional manner by mixing with a pharmaceutically acceptable carrier or diluent. Unit dosage forms for oral administration contain, e.g., from about 0.1 mg to about 500 mg of active substance. Topical administration is, e.g., to the skin. A further form of topical administration is to the eye. Compounds may be administered in free form or in a pharmaceutically acceptable salt form. Such salts may be prepared in a conventional manner and exhibit the same order of activity as the free compounds.

The present invention enables the use of molecular design techniques, particularly the rational drug design approach, to prepare new or improved chemical entities and compounds, including c-Abl inhibitors capable of irreversibly or reversibly modulating c-Abl activity. Improved entities or compounds means that these entities or compounds are superior to the “original” or parent compound they are derived from with regard to a property relevant to therapeutic use including suitability for in vivo administration, e.g., cellular uptake, solubility, stability against (enzymatic) degradation, binding affinity or specificity and the like. For example, on the basis of the information provided herein it is possible to specially design c-Abl inhibitors which covalently, or preferably non-covalently, bind to c-Abl. Such inhibitors may act in a competitive or uncompetitive manner, bind at or close to the active site of c-Abl or act allosterically.

With regard to the design of c-Abl modulators the following aspects should be considered:
(i) if the candidate compound is capable of physically and structurally associating with c-Abl; and/or
(ii) if the compound is capable of assuming a conformation allowing it to associate with c-Abl.

Advantageously, computer modeling techniques may be used in the process of assessing these abilities for the modulator as a whole, or a fragment thereof in order to minimize efforts in the synthesis or testing of unsuccessful candidate compounds. To this end specialized computer software may be used and is well-known in the art.

Another design approach is to probe a c-Abl crystal with a variety of different chemical entities to determine optimal sites for interaction between candidate c-Abl inhibitors and the target kinase. Yet another possibility which arises from the present invention is to screen computationally small molecule data bases for chemical entities or compounds that are capable of binding, in whole or in part, to a c-Abl catalytic domain. The quality of fit to the binding site may be judged, e.g., by shape complementarity or by estimated interaction energy. Knowledge of the three-dimensional arrangement of the modifications can then be utilized for the design of new c-Abl ligands or low molecular weight compounds, such as selective inhibitors.

Chemical entities that are capable of associating with c-Abl may inhibit its interaction with naturally-occurring ligands of the protein and may inhibit biological functions mediated by such interaction. Such chemical entities are potential drug candidates. Compounds of the structures selected or designed by any of the foregoing means may be tested for their ability to bind to c-Abl and to inhibit the binding to a natural or non-natural ligand thereof, and/or inhibit a biological function mediated by c-Abl.

The following examples serve to illustrate embodiments of the present invention but should not be construed as a limitation thereof.

In practicing the present invention, many conventional techniques in molecular biology are used. These techniques are well-known and are explained in, e.g., the following texts, the contents of which are hereby incorporated by reference. See Current Protocols in Molecular Biology, Vols. I-III, Ausubel, Ed. (1997); Sambrook et al., Molecular Cloning: A Laboratory Manual, 2^{nd} Edition, Cold Spring Harbor Laboratory Press, Cold Spring Harbor, N.Y. (1989); DNA Cloning: A Practical Approach, Vols. I and II, Glover, Ed. (1985); Oligonucleotide Synthesis, Gait, Ed. (1984); Nucleic Acid Hybridization, Hames and Higgins, Eds. (1985); Transcription and Translation, Hames and Higgins, Eds. (1984); Animal Cell Culture, Freshney, Ed. (1986); Immobilized Cells and Enzymes, IRL Press (1986); A Practical Guide to Molecular Cloning; the series, Methods in Enzymology, Perbal, Ed., Academic Press, Inc. (1984); Gene Transfer Vectors for Mammalian Cells, Miller and Calos, Eds., Cold Spring Harbor Laboratory (1987); and Methods in Enzymology, Vols. 154 and 155, Wu and Grossman, and Wu, Eds., respectively (1987). In addition, unless noted otherwise, the manufacturer's recommendations are followed when using commercially-available kits.

### EXAMPLE 1

Generation of c-Abl for Expression in Insect Cells

The DNA construct comprising the kinase domain of c-Abl is cloned and expressed in the baculovirus/Sf9 system as described in Bhat et al., J Biol Chem, Vol. 27, pp. 16170-16175 (1997). Cell culture and propagation is performed as described therein with minor modifications. Specifically, Sf9 cells are grown in Excell 400 medium (JRH Biosciences) containing 5% (v/v) fetal calf serum (fcs) in Techne spinner bottles at 28° C. with rotation at 80 rpm.

To generate a high titer virus stock, an amplification protocol is applied where stepwise the Sf9 cell amounts and Excell 400 medium volumes, as well as the infecting virus supernatant volumes, are increased until a virus stock of 500 mL is generated.

Expression of recombinant c-Abl protein in Sf9 cells is carried out in a 8 L total volume bioreactor (FairMenTec GmbH, Germany). Cells are seeded at a density of 2×10⁵ cells/mL in Excell 400 medium+5% fcs and grown to a density of 2.5×10⁶ cells/mL in a volume of 5 L. The O₂ level is maintained at 36% saturation by oxygen-sensor (Cole Parmer) driven addition of O₂ as required. The culture is stirred with a pitch-blade impeller at a speed of 125 rpm throughout the culture time. The pH of the culture generally remains constant during this time when Excell 400 medium is used. The culture temperature is maintained at 28° C. by a water-jacket surrounding the culture vessel. Compressed air is added to the headroom to prevent foaming. Recombinant baculovirus harboring the gene encoding a His-tagged fragment (amino acids P218-S500) of human Abl-kinase (Swiss Prot Accession No. P00519; and Isoform IA, see Tables 1 and 2, below) is added to give a multiplicity of infection (m.o.i.) of 10.

TABLE 1
Swiss Prot Accession No. P00519; Isoform IA of Human c-Abl

[SEQ ID NO. 1]

```
                10 20 30 40 50 60
                  | | | | | |
MLEICLKLVG CKSKKGLSSS SSCYLEEALQ RPVASDFEPQ GLSEAARWNS KENLLAGPSE
                70 80 90 100 110 120
                  | | | | | |
NDPNLFVALY DFVASGDNTL SITKGEKLRV LGYNHNGEWC EAQTKNGQGW VPSNYITPVN
              130 140 150 160 170 180
                  | | | | | |
SLEKHSWYHG PVSRNAAEYL LSSGINGSFL VRESESSPGQ RSISLRYEGR VYHYRINTAS
              190 200 210 220 230 240
                  | | | | | |
DGKLYVSSES RFNTLAELVH HHSTVADGLI TTLHYPAPKR NKPTVYGVSP NYDKWEMERT
              250 260 270 280 290 300
                  | | | | | |
DITMKHKLGG GQYGEVYEGV WKKYSLTVAV KTLKEDTMEV EEFLKEAAVM KEIKHPNLVQ
              310 320 330 340 350 360
                  | | | | | |
LLGVCTREPP FYIITEFMTY GNLLDYLREC NRQEVNAVVL LYMATQTSSA MEYLEKKNFI
              370 380 390 400 410 420
                  | | | | | |
HRDLAARNCL VGENHLVKVA DFGLSRLMTG DTYTAHAGAK FPIKWTAPES LAYNKESIKS
              430 440 450 460 470 480
                  | | | | | |
DVWAFGVLLW EIATYGMSPY PGIDLSQVYE LLEKDYRMER PEGCPEKVYE LMRACWQWNP
              490 500 510 520 530 540
                  | | | | | |
SDRPSFAEIH QAFETMFQES SISDEVEKEL GKQGVRGAVS TLLQAPELPT KTRTSRRAAE
              550 560 570 580 590 600
                  | | | | | |
HRDTTDVPEM PHSKGQGESD PLDHEPAVSP LLPRKERGPP EGGLNEDERL LPKDKKTNLF
              610 620 630 640 650 660
                  | | | | | |
SALIKKKKKT APTPPKRSSS FRENDGQPER RGAGEEEGRD ISNGALAFTP LDTADPAKSP
              670 680 690 700 710 720
                  | | | | | |
KPSNGAGVPN GALRESGGSG FRSPHLWKKS STLTSSRLAT GEEEGGGSSS KRFLRSCSAS
              730 740 750 760 770 780
                  | | | | | |
CVPHGAKDTE WRSVTLPRDL QSTGRQFDSS TFGGHKSEKP ALPRKRAGEN RSDQVTRGTV
              790 800 810 820 830 840
                  | | | | | |
TPPPRLVKKN EEAADEVFKD IMESSPGSSP PNLTPKPLRR QVTVAPASGL PHKEEAEKGS
              850 860 870 880 890 900
                  | | | | | |
ALGTPAAAEP VTPTSKAGSG APGGTSKGPA EESRVRRHKH SSESPGRDKG KLSRLKPAPP
              910 920 930 940 950 960
                  | | | | | |
PPPAASAGKA GGKPSQSPSQ EAAGEAVLGA KTKATSLVDA VNSDAAKPSQ PGEGLKKPVL
              970 980 990 1000 1010 1020
                  | | | | | |
PATPKPQSAK PSGTPISPAP VPSTLPSASS ALAGDQPSST AFIPLISTRV SLRKTRQPPE
            1030 1040 1050 1060 1070 1080
                  | | | | | |
RIASGATTKG VVLDSTEALC LAISRNSEQM ASHSAVLEAG KNLYTFCVSY VDSTQQMRNK
            1090 1100 1110 1120 1130
                  | | | | |
FAFREAINKL ENNLRELQIC PATAGSGPAA TQDFSKLLSS VKETSDIVQR
```

The c-Abl polypeptide fragment used in this invention comprises the kinase domain (proline 218 to serine 500) and also comprises an additional stretch of 27 amino acids at the N-terminus comprising a 6×His stretch to allow purification by immobilized metal affinity chromatography (IMAC), as well as a cleavage site for Factor Xa protease digestion (IEGR) to allow removal of the first 23 amino acids prior to the final purification (see SEQ ID NO. 2 and Table 2 below).

[SEQ ID NO 2]

MGYPYDVPDY AMAHHHHHHI EGRAAMD

TABLE 2
c-Abl Kinase Fragment, Amino Acids 218-500

[SEQ ID NO. 3]

```
              Factor Xa Papain
              ↓ 218 ↓
MGYPYDVPDY AMAHHHHHHI EGRAAMDPKR NKPTVYGVSP
NYDKWEMERT DITMKHKLGG GQYGEVYEGV WKKYSLTVAV KTLKEDTMEV EEFLKEAAVM
KEIKHPNLVQ LLGVCTREPP FYIITEFMTY GNLLDYLREC NRQEVNAVVL LYMATQISSA
MEYLEKKNFT HRDLAARNCL VGENHLVKVA DFGLSRLMTG DTYTAHAGAK FPIKWTAPES
LAYNKFSIKS DVWAFGVLLW
EIATYGMSPY PGTDLSQVYE LLEKDYRMER PEGCPEKVYE LMRACWQWNP SDRPSFAEIH
QAFETMFQES
```

While a c-Abl protein fragment may be obtained by Factor Xa cleavage, the fragment V228 to S500 obtained by cleavage of SEQ ID NO. 3 with the protease papain is found to crystallize much better than the one obtained from Factor Xa cleavage. Thus, in the present invention, the segment of the human c-Abl protein crystallized in this invention is a fragment of the kinase domain from V228 to S500 (SEQ ID NO. 4) obtained by cleavage with the protease papain. A total of 37 amino acids are removed from the N-terminus of the original construct which is expressed in insect cells and the c-Abl protein fragment obtained from papain cleavage does not contain any amino acid not originating from the human c-Abl sequence.

Human c-abl Kinase Domain (V228 to S500)

[SEQ ID NO. 4]
VSPNYDKWEM ERTDITMKHK LGGGQYGEVY EGVWKKYSLT VAVKTLKEDT MEVEEFLKEA
AVMKETKHPN LVQLLGVCTR EPPFYIITEF MTYGNLLDYL RECNRQEVNA VVLLYMATQI
SSANEYLEKK NFIHRDLAAR NCLVGENHLV KVADFGLSRL MTGDTYTAHA GAKFPIKWTA
PESLAYNKES IKSDVWAFGV LLWEIATYGM SPYPGIDLSQ VYELLEKDYR MERPEGCPEK
VYELMRACWQ WNPSDRPSFA EIHQAFETMF QES

Forty-eight (48) hours after infection, Compound A (discussed below) (10 mM in DMSO) is added to the culture to achieve a final concentration of 3 μM. Twenty-four (24) hours later, cells with a density of 1.5×10⁶ are harvested by centrifugation and the cell pellet stored at −70° C.

Compound A is added to allow complex formation between the c-Abl protein and a compound known to bind to the active site of the protein. Compound A is a polycyclic compound with anti-tumor properties and its structure and synthesis is disclosed in Example 1 of WO 99/02532, the contents of which are hereby incorporated by reference. The chemical structure of Compound A is provided below: 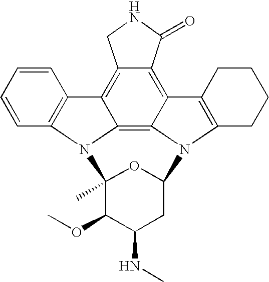
Purification

Cells from one 5 L bioreactor (7.5×10⁹ cells total) are lysed in 180 mL of buffer A (50 mM Na-phosphate, 300 mM NaCl, 10 vol % glycerol, 10 mM 2-mercaptoethanol, pH 8) supplemented with a protease inhibitor cocktail (Complete™, EDTA-free, Roche) and 20 μM Compound A (tartrate salt) by sonication at 4-10° C. The lysate is clarified by centrifugation at 45,000×g and 4° C. for 100 minutes and is additionally filtered through a 0.45 μM membrane (Stericup, Millipore). The filtrate (250 mL) is adjusted to pH 8 with NaOH and after addition of 0.5 mL Compound A (10 mM in DMSO) is loaded onto a 18 mL column of Ni-NTA superflow resin (Quiagen) at a flow rate of 100 mL/hour at 4° C. After loading the column is subsequently washed with buffer A containing 10 mM and 15 mM imidazole to remove contaminating proteins. After an additional wash with 15 mM imidazole in buffer B (20 mM tris, 100 mM NaCl, 10 vol % glycerol, 5 mM 2-mercaptoethanol, pH 8 adjusted with HCl at 4° C.) the bound His-Abl is eluted with 50 mL of 100 mM imidazole in buffer B at a flow rate of 60 mL/hour. HPLC analysis indicates that the buffer B wash contains about 6 mg and the 100 mM imidazole eluate about 13 mg His-Abl and co-elution of Compound A is not detectable. Analysis by mass spectrometry indicates that about 50% of the isolated His-Abl is un-phosphorylated, 35% mono- and 15% di-phosphorylated. After addition of 1 mM EDTA, 2 mM DTT and 20 μM Compound A (final concentrations) both solutions are concentrated to 5 mL and 13 mL, respectively and combined. HPLC-analysis of this pool shows a concentration of about 1 mg c-Abl/mL and a molar ratio of c-Abl to Compound A of about 1:1. To remove the His-tag together with 10 amino acids from the N-terminus of c-Abl (amino acids 218-227) 18.5 μg of papain (Roche) is added and the reaction allowed to proceed at 4° C. for 4 hours Papain is inactivated by addition of anti-pain (Roche, 25 μg/mL final concentration). Five hundred (500) units of T-cell protein tyrosine phosphate (New England Biolabs) is added and incubation continued for additional 19 hours at 4° C. to dephosphorylate c-Abl. Compound A is then added to achieve a 2-fold molar excess and the reaction concentrated to 3.5 mL by ultratiltration in a centrifugal filter device with a 10 KDa MW cut-off (Amicon). The concentrate containing about 15 mg of c-Abl and a 1.25 molar excess of Compound A is loaded onto a HiLoad 16/60 Superdex 75 prep grade column (Amersham Biosciences) run in 20 mM Tris, 100 mM NaCl, 0.2 M ethyleneglycol, 1 mM EDTA, 3 mM DTT, pH 7.6 at a flow rate of 1.3 mL/min. The column is cooled by pumping ice water through the column jacket. c-Abl elutes as symmetrical peak at a elution volume corresponding to the monomeric protein, but most of Compound A is lost during size exclusion chromatography. To the Superdex 75 fractions (total 12 mL, 14 mg c-abl) is added 50 μM of Compound A to achieve a 2-fold molar excess over c-Abl, the solution concentrated to 0.6 mL by ultrafiltration and the concentrate centrifuged to remove a small amount of precipitate. HPLC analysis indicates a concentration of 24-25 mg c-Abl/mL and a molar ratio of c-Abl: Compound A of about 1:0.95. The purified c-Abl kinase is at least 95% pure judged by reducing, Coomassie stained SDS-PAGE and by reversed-phase HPLC. Mass spectrometry however shows that about 12% of the protein is still phosphorylated (expected mass: 31 642.3 Da for c-Abl (V228 to S500) unphosphorylated; measured: 31 641.9 Da (−88%); 31 721.5 Da (−12%), +79.6 Da, monophosphorylated). The c-Abl/Compound A complex at this stage of purity and homogeneity is used for crystallization.

### EXAMPLE 2

Structure Determination of Human Abl Kinase in Complex with Compound A at 2.8 Å Resolution

Crystallization

Abl protein complexed with Compound A (in ˜24 mg/mL in 20 mM Tris HCl, 100 mM NaCl, 1 mM EDTA, 0.2 M ethylene glycol, pH 7.6, +3 mM DTT) produced as disclosed above is used for crystallization. Crystal growth is obtained after incubation of 1 μL of protein solution with 1 μL of reservoir solution (12% PEG 8000 (w/v), 0.2 M MgAcetate, 0.1 M HEPES pH 7.5) and equilibration through the vapor phase with 1,000 μL of the reservoir solution (12% PEG 8,000 (w/v), 0.2 M MgAcetate, 0.1 M HEPES pH 7.5) at 4° C. using methods familiar to one of skill in the art.

Data Collection

The crystals are gradually transferred over a period of 2 hours to 12% PEG 8,000 (w/v), 0.2 M MgAcetate, 0.1 M HEPES pH 7.5, 25% glycerol, then cryo cooled by dunking in liquid nitrogen and stored in liquid nitrogen. Data are collected at beamline X06SA [Swiss Light Source (SLS) Villigen, Switzerland]. The wavelength of the X-rays is 0.977789 Å and the beam current is 300 mA. The crystal to detector distance is 200 mm and the rotation range for each image is 0.75° with an exposure time of 1.5 seconds, 280 images are collected, but only 214 are used in data processing because radiation damage is obvious in the last images. Data are processed with the HKL package (Zbyszek Otwinowski, University of Texas Southwestern Medical Center, Dallas) and statistics are listed in Table 3.

TABLE 3
Data Collection Statistics

| Crystal Size | 0.06 × 0.06 × 0.015 mm³ |
|---|---|
| Number of crystals used for data set | 1 |
| Space group | C2 |
| Unit cell (a, b, c, beta) | 182.56 Å, 58.33 Å, |
| | 103.99 Å, 119.16° |
| Images used | 214 (≡160.5°) |
| Measurements (each partial) | 291875 |
| Unique measurements (each reflection) | 63157 |
| Unique reflections | 22731 |
| Multiplicity | 2.8 |
| Resolution range | 40.0-2.8 Å |
| Refined mosaicity | 1.25° |
| Completeness (shell 2.9-2.8 Å) | 96.2% (83.3%) |
| Rmerge (shell 2.9-2.8 Å) | 0.087 (0.335) |
| Percentage of data with l/sig(l) ≧ 2 | 77.3 |

Structure Determination and Refinement

Molecular replacement is performed using Abl kinase amino acids 235-380 and 401-498 (see Table 4 below).

TABLE 4
Amino Acid Sequence for Molecular Replacement Model

[SEQ ID NO. 5]

235-380:
WEMERTDITM KHKLGGGQYG EVYEGVWKKY SLTVAVKTLK EDTMEVEEFL KEAAVMKEIK
HPNLVQLLGV CTREPPFYII TEFMTYGNLL DYLRECNRQE VSAVVLLYMA TQISSAMEYL
EKKNFIHRDL AARNCLVGEN HLVKVA

[SEQ ID NO. 6]

401-498:
FPIK WTAPESLAYN KFSIKSDVWA FGVLLWEIAT
YGMSPYPGID LSQVYELLEK DYRMERPEGC PEKVYELMRA CWQWNPSDRP SFAEIHQAFE
TMFQ

Based on the cell volume, the expected number of molecules in the asymmetric unit is 3 (52.6% solvent). Molecular replacement (using MolRep, “MOLREP: an automated program for molecular replacement” [see Vagin and Teplyakov, J Appl Cryst, Vol. 30, pp. 1022-1025 (1997)], within the CCP4 suite “The CCP4 suite: Programs for Protein Crystallography” [see Collaborative Computational Project Number 4, Acta Cryst, Vol. D50, pp. 760-763 (1994) gives 2 strong and one weaker peak in the rotation function and a clearly correct solution after placing of all three molecules with the translation function (R-factor 0.485, correlation 0.422, resolution range 40.0-4.0 Å). Refinement is carried out with alternate cycles of model building on the graphics display using the program 0 [see Jones et al., Acta Cryst, vol. A47, pp. 110-119 (1991)] and restrained maximum likelihood refinement with Refmac5. See Murshudov et al., Acta Cryst, Vol. D53, pp. 240-255 (1997).

Tight NCS restraints are used in the initial cycles, but due to differences between the three molecules in the asymmetric unit, these are taken off in the last cycles. Several water molecules are identified from strong difference density and three molecules of a compound referred to herein as Compound A are found and added to the model.

A parameterization of the translation, libration and screw-rotation displacements of all three protein molecules in the asymmetric unit of the crystals is used in the final refinement cycles. See Winn, Isupov and Murshudov, Acta Cryst, Vol. D57 (2001). A summary of the refinement statistics is listed in Table 5.

TABLE 5
Refinement Statistics

| Contents of model: | |
|---|---|
| Abl kinase | Residues 234-248, 254-500 (2139 |
| (chain id A) | atoms, av. B = 62.6 Å²) |
| Abl kinase | Residues 233-248, 254-498 (2134 |
| (chain id B) | atoms, av. B = 56.6 Å²) |
| Abl kinase | Residues 234-248, 254-264, 267-274, |
| (chain id C) | 276-500 (2115 atoms, av. B = 78.2 Å²) |
| Compound A | 3 × 35 atoms (av. B = |
| (chains I, J, K) | 47.7 Å², 67.9 Å², 81.0 Å²) |
| Water | 19 atoms (av. B = 51.5 Å²) |
| (chain id W) | |
| R-value | 0.218 |
| Free R-value | 0.287 |
| Mean B-value | 63.29 Å² |
| r.m.s.d values: | |
| bonds | 0.020 Å |
| angles | 1.86° |
| Residues in allowed | 99.6% (86.3%) |
| region of | |
| Ramachandran | |
| plot (core) | |

Overall Structure

The final structure of Abl in these crystals contains two molecules of Abl kinase with empty ATP binding sites (chains A and C) and one molecule (chain B) with Compound A (chain I) bound in the ATP binding site. The other two Compound A molecules (chains K and J) are hydrogen bonded to the C-terminus of molecules A and C respectively, and are held in place by crystal contacts with other Abl kinase domains. Molecules A and C are related by a local two-fold symmetry axis.

The conformation of all three Abl kinase domains is very similar. There is some shift of the N-terminal lobe with respect to the C-terminal lobe, but the positions of the residues important for catalysis are very similar. A superposition with the activated Lck kinase domain [see Yamaguchi and Hendrickson (1996), supra] shows that Abl is in an active conformation (data not shown). The activation loop including the DFG motif, despite the lack of phosphorylation, follows the same path as in Lck and the catalytic residues, e.g., from helix C, are all correctly positioned.

TABLE 6
Atomic Coordinates for c-Abl Crystal Structure

| HEADER | | ---- | XX-XXX-XX xxxx | |
|---|---|---|---|---|
| COMPND | | --- | | |
| REMARK | 3 | | | |
| REMARK | 3 | REFINEMENT. | | |
| REMARK | 3 | PROGRAM: | REFMAC 5.1.9999 | |
| REMARK | 3 | AUTHORS: | MURSHUDOV, | |
| REMARK | 3 | VAGIN, | | |
| REMARK | 3 | DODSON | | |
| REMARK | 3 | | | |
| REMARK | 3 | REFINEMENT TARGET: | MAXIMUM | |
| REMARK | 3 | LIKELIHOOD | | |
| REMARK | 3 | | | |
| REMARK | 3 | DATA USED IN REFINEMENT. | | |
| REMARK | 3 | RESOLUTION RANGE HIGH (ANGSTROMS): | 2.80 | |
| REMARK | 3 | RESOLUTION RANGE LOW (ANGSTROMS): | 40.00 | |
| REMARK | 3 | DATA CUTOFF (SIGMA(F)): | NONE | |
| REMARK | 3 | COMPLETENESS FOR RANGE | (%): | 96.24 |
| REMARK | 3 | NUMBER OF REFLECTIONS: | 22372 | |
| REMARK | 3 | | | |
| REMARK | 3 | FIT TO DATA USED IN REFINEMENT. | | |
| REMARK | 3 | CROSS-VALIDATION METHOD: | THROUGHOUT | |
| REMARK | 3 | FREE R VALUE TEST SET SELECTION: | RANDOM | |
| REMARK | 3 | R VALUE (WORKING + TEST SET): | 0.21990 | |
| REMARK | 3 | R VALUE (WORKING SET): | 0.21644 | |
| REMARK | 3 | FREE R VALUE: | 0.28505 | |
| REMARK | 3 | FREE R VALUE TEST SET SIZE (%): | 5.1 | |
| REMARK | 3 | FREE R VALUE TEST SET COUNT: | 1203 | |
| REMARK | 3 | | | |
| REMARK | 3 | FIT IN THE HIGHEST RESOLUTION BIN. | | |
| REMARK | 3 | TOTAL NUMBER OF BINS USED: | 20 | |
| REMARK | 3 | BIN RESOLUTION RANGE HIGH: | 2.800 | |
| REMARK | 3 | BIN RESOLUTION RANGE LOW: | 2.872 | |
| REMARK | 3 | REFLECTION IN BIN (WORKING SET): | 1330 | |
| REMARK | 3 | BIN R VALUE (WORKING SET): | 0.335 | |
| REMARK | 3 | BIN FREE R VALUE SET COUNT: | 60 | |
| REMARK | 3 | BIN FREE R VALUE: | 0.399 | |
| REMARK | 3 | | | |
| REMARK | 3 | NUMBER OF NON-HYDROGEN ATOMS USED IN REFINEMENT. | | |
| REMARK | 3 | ALL ATOMS: | 6518 | |
| REMARK | 3 | | | |
| REMARK | 3 | B VALUES. | | |
| REMARK | 3 | FROM WILSON PLOT (A**2): | NULL | |
| REMARK | 3 | MEAN B VALUE (OVERALL, A**2): | 63.614 | |
| REMARK | 3 | OVERALL ANISOTROPIC B VALUE. | | |

| | | | |
|---|---|---|---|
| REMARK | 3 | B11 (A**2): | −1.42 |
| REMARK | 3 | B22 (A**2): | 6.00 |
| REMARK | 3 | B33 (A**2): | −5.84 |
| REMARK | 3 | B12 (A**2): | 0.00 |
| REMARK | 3 | B13 (A**2): | −1.30 |
| REMARK | 3 | B23 (A**2): | 0.00 |
| REMARK | 3 | | |

| | | | |
|---|---|---|---|
| REMARK | 3 | ESTIMATED OVERALL COORDINATE ERROR. | |
| REMARK | 3 | ESU BASED ON R VALUE (A): | NULL |
| REMARK | 3 | ESU BASED ON FREE R VALUE (A): | 0.444 |
| REMARK | 3 | ESU BASED ON MAXIMUM LIKELIHOOD (A): | 0.326 |
| REMARK | 3 | ESU FOR B VALUES BASED ON MAXIMUM LIKELIHOOD (A**2): | 36.309 |
| REMARK | 3 | | |
| REMARK | 3 | CORRELATION COEFFICIENTS. | |
| REMARK | 3 | CORRELATION COEFFICIENT FO-FC: | 0.935 |
| REMARK | 3 | CORRELATION COEFFICIENT FO-FC FREE: | 0.885 |
| REMARK | 3 | | |

| | | | | | |
|---|---|---|---|---|---|
| REMARK | 3 | RMS DEVIATIONS FROM IDEAL VALUES | COUNT | RMS | WEIGHT |
| REMARK | 3 | BOND LENGTHS REFINED ATOMS (A): | 6680 | 0.020 | 0.022 |
| REMARK | 3 | BOND LENGTHS OTHERS (A): | 5965 | 0.003 | 0.020 |
| REMARK | 3 | BOND ANGLES REFINED ATOMS (DEGREES): | 9067 | 1.869 | 1.966 |
| REMARK | 3 | BOND ANGLES OTHERS (DEGREES): | 13898 | 1.315 | 3.000 |
| REMARK | 3 | TORSION ANGLES, PERIOD 1 (DEGREES): | 771 | 8.781 | 5.000 |
| REMARK | 3 | TORSION ANGLES, PERIOD 2 (DEGREES): | 308 | 39.831 | 24.156 |
| REMARK | 3 | TORSION ANGLES, PERIOD 3 (DEGREES): | 1169 | 18.050 | 15.000 |
| REMARK | 3 | TORSION ANGLES, PERIOD 4 (DEGREES): | 33 | 19.175 | 15.000 |
| REMARK | 3 | CHIRAL-CENTER RESTRAINTS (A**3): | 964 | 0.102 | 0.200 |
| REMARK | 3 | GENERAL PLANES REFINED ATOMS (A): | 7218 | 0.007 | 0.020 |
| REMARK | 3 | GENERAL PLANES OTHERS (A): | 1365 | 0.003 | 0.020 |
| REMARK | 3 | NON-BONDED CONTACTS REFINED ATOMS (A): | 1317 | 0.189 | 0.200 |
| REMARK | 3 | NON-BONDED CONTACTS OTHERS (A): | 5686 | 0.130 | 0.200 |
| REMARK | 3 | NON-BONDED TORSION OTHERS (A): | 3612 | 0.077 | 0.200 |
| REMARK | 3 | H-BOND (X. . .Y) REFINED ATOMS (A): | 194 | 0.137 | 0.200 |
| REMARK | 3 | SYMMETRY VDW REFINED ATOMS (A): | 22 | 0.141 | 0.200 |
| REMARK | 3 | SYMMETRY VDW OTHERS (A): | 64 | 0.142 | 0.200 |
| REMARK | 3 | SYMMETRY H-BOND REFINED ATOMS (A): | 7 | 0.159 | 0.200 |
| REMARK | 3 | | | | |
| REMARK | 3 | ISOTROPIC THERMAL FACTOR RESTRAINTS. | COUNT | RMS | WEIGHT |
| REMARK | 3 | MAIN-CHAIN BOND REFINED ATOMS (A**2): | 4498 | 0.765 | 1.500 |
| REMARK | 3 | MAIN-CHAIN BOND OTHER ATOMS (A**2): | 1573 | 0.122 | 1.500 |
| REMARK | 3 | MAIN-CHAIN ANGLE REFINED ATOMS (A**2): | 6297 | 1.100 | 2.000 |
| REMARK | 3 | SIDE-CHAIN BOND REFINED ATOMS (A**2): | 3667 | 1.368 | 3.000 |
| REMARK | 3 | SIDE-CHAIN ANGLE REFINED ATOMS (A**2): | 2770 | 2.264 | 4.500 |
| REMARK | 3 | | | | |

| | | | |
|---|---|---|---|
| REMARK | 3 | NCS RESTRAINTS STATISTICS | |
| REMARK | 3 | NUMBER OF NCS GROUPS: NULL | |
| REMARK | 3 | | |
| REMARK | 3 | | |
| REMARK | 3 | TLS DETAILS | |
| REMARK | 3 | NUMBER OF TLS GROUPS: 6 | |
| REMARK | 3 | | |
| REMARK | 3 | TLS GROUP: 1 | |
| REMARK | 3 | NUMBER OF COMPONENTS GROUP: 1 | |
| REMARK | 3 | COMPONENTS | C SSSEQI TO C SSSEQI |
| REMARK | 3 | RESIDUE RANGE: | A 234 A 318 |

| | | | | |
|---|---|---|---|---|
| REMARK | 3 | ORIGIN FOR THE GROUP (A): 33.1740 | 0.3010 | 37.1720 |
| REMARK | 3 | T TENSOR | | |

| | | | | | |
|---|---|---|---|---|---|
| REMARK | 3 | T11: | −0.1258 | T22: | 0.1688 |
| REMARK | 3 | T33: | −0.1102 | T12: | 0.0875 |
| REMARK | 3 | T13: | −0.0254 | T23: | −0.0041 |

| | | |
|---|---|---|
| REMARK | 3 | L TENSOR |

| | | | | | |
|---|---|---|---|---|---|
| REMARK | 3 | L11: | 4.2002 | L22: | 4.5001 |
| REMARK | 3 | L33: | 4.3983 | L12: | 0.4480 |
| REMARK | 3 | L13: | −0.7500 | L23: | −0.3640 |

| | | |
|---|---|---|
| REMARK | 3 | S TENSOR |

| | | | | | | | |
|---|---|---|---|---|---|---|---|
| REMARK | 3 | S11: | 0.3150 | S12: | −0.2302 | S13: | −0.0056 |
| REMARK | 3 | S21: | 0.6499 | S22: | −0.1337 | S23: | 0.4308 |
| REMARK | 3 | S31: | −0.2533 | S32: | −0.4923 | S33: | −0.1813 |
| REMARK | 3 | | | | | | |

| | | | |
|---|---|---|---|
| REMARK | 3 | TLS GROUP : 2 | |
| REMARK | 3 | NUMBER OF COMPONENTS GROUP: 2 | |
| REMARK | 3 | COMPONENTS | C SSSEQI TO C SSSEQI |
| REMARK | 3 | RESIDUE RANGE: | A 319 A 500 |
| REMARK | 3 | RESIDUE RANGE: | I 501 I 501 |

| | | | | | |
|---|---|---|---|---|---|
| REMARK | 3 | ORIGIN FOR THE GROUP (A): | 57.0230 | 0.5150 | 27.0400 |
| REMARK | 3 | T TENSOR | | | |

| | | | | | |
|---|---|---|---|---|---|
| REMARK | 3 | T11: | 0.0306 | T22: | −0.1866 |
| REMARK | 3 | T33: | 0.0244 | T12: | 0.0591 |
| REMARK | 3 | T13: | −0.0248 | T23: | 0.0154 |

| | | |
|---|---|---|
| REMARK | 3 | L TENSOR |

| | | | | | |
|---|---|---|---|---|---|
| REMARK | 3 | L11: | 1.7326 | L22: | 1.4053 |
| REMARK | 3 | L33: | 1.5694 | L12: | −0.4819 |
| REMARK | 3 | L13: | −1.0195 | L23: | 0.6939 |

| | | |
|---|---|---|
| REMARK | 3 | S TENSOR |

| | | | | | | | |
|---|---|---|---|---|---|---|---|
| REMARK | 3 | S11: | 0.1908 | S12: | 0.2970 | S13: | −0.0068 |
| REMARK | 3 | S21: | −0.1108 | S22: | 0.1466 | S23: | 0.0678 |
| REMARK | 3 | S31: | −0.1679 | S32: | 0.3014 | S33: | 0.0442 |
| REMARK | 3 | | | | | | |

| | | | |
|---|---|---|---|
| REMARK | 3 | TLS GROUP : 3 | |
| REMARK | 3 | NUMBER OF COMPONENTS GROUP: 1 | |
| REMARK | 3 | COMPONENTS | C SSSEQI TO C SSSEQI |
| REMARK | 3 | RESIDUE RANGE: | B 233 B 318 |

| | | | | | |
|---|---|---|---|---|---|
| REMARK | 3 | ORIGIN FOR THE GROUP (A): | 42.7060 | 12.2540 | 65.8540 |
| REMARK | 3 | T TENSOR | | | |

| | | | | | |
|---|---|---|---|---|---|
| REMARK | 3 | T11: | 0.0983 | T22: | −0.2988 |
| REMARK | 3 | T33: | 0.1021 | T12: | −0.0617 |
| REMARK | 3 | T13: | 0.0974 | T23: | −0.0054 |

| | | |
|---|---|---|
| REMARK | 3 | L TENSOR |

| | | | | | |
|---|---|---|---|---|---|
| REMARK | 3 | L11: | 4.3243 | L22: | 5.7869 |
| REMARK | 3 | L33: | 3.8414 | L12: | 0.1164 |
| REMARK | 3 | L13: | −0.8713 | L23: | 0.5655 |

| | | |
|---|---|---|
| REMARK | 3 | S TENSOR |

| | | | | | | | |
|---|---|---|---|---|---|---|---|
| REMARK | 3 | S11: | −0.2374 | S12: | 0.5340 | S13: | 0.5608 |
| REMARK | 3 | S21: | 0.7627 | S22: | −0.0670 | S23: | 0.2730 |
| REMARK | 3 | S31: | 0.3596 | S32: | −0.2658 | S33: | 0.3043 |
| REMARK | 3 | | | | | | |

| | | | |
|---|---|---|---|
| REMARK | 3 | TLS GROUP : 4 | |
| REMARK | 3 | NUMBER OF COMPONENTS GROUP: 2 | |
| REMARK | 3 | COMPONENTS | C SSSEQI TO C SSSEQI |
| REMARK | 3 | RESIDUE RANGE: | B 319 B 498 |
| REMARK | 3 | RESIDUE RANGE: | J 501 J 501 |

| | | | | | |
|---|---|---|---|---|---|
| REMARK | 3 | ORIGIN FOR THE GROUP (A): | 49.06 | 33.2100 | 53.2640 |
| REMARK | 3 | T TENSOR | | | |

| | | | | | |
|---|---|---|---|---|---|
| REMARK | 3 | T11: | 0.0147 | T22: | ta,32 −0.1809 |
| REMARK | 3 | T33: | 0.0790 | T12: | 0.0262 |
| REMARK | 3 | T13: | −0.0005 | T23: | 0.0182 |

| | | |
|---|---|---|
| REMARK | 3 | L TENSOR |

| | | | | | |
|---|---|---|---|---|---|
| REMARK | 3 | L11: | 0.7064 | L22: | 1.3543 |
| REMARK | 3 | L33: | 0.8307 | L12: | 0.2355 |
| REMARK | 3 | L13: | 0.5106 | L23: | 0.4316 |

| | | |
|---|---|---|
| REMARK | 3 | S TENSOR |

| | | | | | | | |
|---|---|---|---|---|---|---|---|
| REMARK | 3 | S11: | 0.0778 | S12: | 0.0331 | S13: | 0.0008 |
| REMARK | 3 | S21: | −0.0287 | S22: | 0.1143 | S23: | 0.0500 |
| REMARK | 3 | S31: | −0.1019 | S32: | 0.1826 | S33: | 0.0366 |
| REMARK | 3 | | | | | | |

| | | | |
|---|---|---|---|
| REMARK | 3 | TLS GROUP: 5 | |
| REMARK | 3 | NUMBER OF COMPONENTS GROUP: 1 | |
| REMARK | 3 | COMPONENTS | C SSSEQI TO C SSSEQI |
| REMARK | 3 | RESIDUE RANGE: | C 234 C 318 |

| | | | | | |
|---|---|---|---|---|---|
| REMARK | 3 | ORIGIN FOR THE GROUP (A): | 38.3990 | −2.2700 | −4.7780 |
| REMARK | 3 | T TENSOR | | | |

| | | | | | |
|---|---|---|---|---|---|
| REMARK | 3 | T11: | 0.0724 | T22: | 0.0739 |
| REMARK | 3 | T33: | −0.0972 | T12: | 0.1733 |
| REMARK | 3 | T13: | 0.0996 | T23: | 0.1977 |

| | | |
|---|---|---|
| REMARK | 3 | L TENSOR |

| | | | | | |
|---|---|---|---|---|---|
| REMARK | 3 | L11: | 3.6132 | L22: | 2.3842 |
| REMARK | 3 | L33: | 3.5724 | L12: | −2.1106 |
| REMARK | 3 | L13: | −1.0922 | L23: | 0.9867 |

| | | |
|---|---|---|
| REMARK | 3 | S TENSOR |

| | | | | | | | |
|---|---|---|---|---|---|---|---|
| REMARK | 3 | S11: | −0.4434 | S12: | −0.4700 | S13: | −0.2869 |
| REMARK | 3 | S21: | −0.1804 | S22: | 0.1448 | S23: | 0.0240 |
| REMARK | 3 | S31: | 0.7706 | S32: | 0.5263 | S33: | 0.2986 |
| REMARK | 3 | | | | | | |

| | | | |
|---|---|---|---|
| REMARK | 3 | TLS GROUP: 6 | |
| REMARK | 3 | NUMBER OF COMPONENTS GROUP: 2 | |
| REMARK | 3 | COMPONENTS: C SSSEQI TO C SSSEQI | |
| REMARK | 3 | RESIDUE RANGE | C 319 C 500 |
| REMARK | 3 | RESIDUE RANGE: | K 501 K 501 |

| | | | | | |
|---|---|---|---|---|---|
| REMARK | 3 | ORIGIN FOR THE GROUP (A): | 19.4870 | 10.3820 | 5.7900 |
| REMARK | 3 | T TENSOR | | | |

| | | | | | |
|---|---|---|---|---|---|
| REMARK | 3 | T11: | −0.0708 | T22: | −0.1500 |
| REMARK | 3 | T33: | −0.1131 | T12: | −0.0134 |
| REMARK | 3 | T13: | −0.0050 | T23: | 0.0785 |

| | | |
|---|---|---|
| REMARK | 3 | L TENSOR |

| | | | | | |
|---|---|---|---|---|---|
| REMARK | 3 | L11: | 5.3830 | L22: | 0.2782 |
| REMARK | 3 | L33: | 9.0399 | L12: | 0.3295 |
| REMARK | 3 | L13: | −5.3853 | L23: | −0.7917 |

| | | |
|---|---|---|
| REMARK | 3 | S TENSOR |

| | | | | | | | |
|---|---|---|---|---|---|---|---|
| REMARK | 3 | S11: | −0.0185 | S12: | −0.3576 | S13: | 0.1481 |
| REMARK | 3 | S21: | −0.0292 | S22: | 0.0844 | S23: | 0.0728 |
| REMARK | 3 | S31: | −0.0448 | S32: | 0.3330 | S33: | −0.0659 |
| REMARK | 3 | | | | | | |
| REMARK | 3 | | | | | | |

| | | |
|---|---|---|
| REMARK | 3 | BULK SOLVENT MODELLING. |
| REMARK | 3 | METHOD USED: BABINET MODEL WITH MASK |
| REMARK | 3 | PARAMETERS FOR MASK CALCULATION |
| REMARK | 3 | VDW PROBE RADIUS: 1.20 |
| REMARK | 3 | ION PROBE RADIUS: 0.80 |
| REMARK | 3 | SHRINKAGE RADIUS: 0.80 |
| REMARK | 3 | |
| REMARK | 3 | OTHER REFINEMENT REMARKS: |
| REMARK | 3 | HYDROGENS HAVE BEEN ADDED IN THE RIDING POSITIONS |
| REMARK | 3 | |

| | | | |
|---|---|---|---|
| LINK | LEU A 248 | GLY A 254 | gap |
| LINK | PRO A 309 | PRO A 310 | PNCIS |
| LINK | LEU B 248 | GLY B 254 | gap |
| LINK | PRO B 309 | PRO B 310 | PNCIS |
| LINK | LEU C 248 | GLY C 254 | gap |
| LINK | TYR C 264 | THR C 267 | gap |
| LINK | LYS C 274 | ASP C 276 | gap |
| LINK | PRO C 309 | PRO C 310 | PNCIS |

| | | |
|---|---|---|
| REMARK | 465 | |
| REMARK | 465 | MISSING RESIDUES |
| REMARK | 465 | THE FOLLOWING RESIDUES WERE NOT LOCATED IN THE |
| REMARK | 465 | EXPERIMENT. (M = MODEL NUMBER; RES = RESIDUE NAME; C = CHAIN |
| REMARK | 465 | IDENTIFIER; SSSEQ = SEQUENCE NUMBER; I = INSERTION CODE.) |
| REMARK | 465 | |

| | | | | |
|---|---|---|---|---|
| REMARK | 465 | M | RES C | SSSEQI |
| REMARK | 465 | | VAL A | 228 |
| REMARK | 465 | | SER A | 229 |
| REMARK | 465 | | PRO A | 230 |
| REMARK | 465 | | ASN A | 231 |
| REMARK | 465 | | TYR A | 232 |
| REMARK | 465 | | ASP A | 233 |
| REMARK | 465 | | GLY A | 249 |
| REMARK | 465 | | GLY A | 250 |
| REMARK | 465 | | GLY A | 251 |
| REMARK | 465 | | GLN A | 252 |
| REMARK | 465 | | TYR A | 253 |
| REMARK | 465 | | VAL B | 228 |
| REMARK | 465 | | SER B | 229 |
| REMARK | 465 | | PRO B | 230 |
| REMARK | 465 | | ASN B | 231 |
| REMARK | 465 | | TYR B | 232 |
| REMARK | 465 | | GLY B | 249 |
| REMARK | 465 | | GLY B | 250 |
| REMARK | 465 | | GLY B | 251 |
| REMARK | 465 | | GLN B | 252 |
| REMARK | 465 | | TYR B | 253 |
| REMARK | 465 | | GLU B | 499 |
| REMARK | 465 | | SER B | 500 |
| REMARK | 465 | | VAL C | 228 |
| REMARK | 465 | | SER C | 229 |
| REMARK | 465 | | PRO C | 230 |
| REMARK | 465 | | ASN C | 231 |
| REMARK | 465 | | TYR C | 232 |
| REMARK | 465 | | ASP C | 233 |
| REMARK | 465 | | GLY C | 249 |
| REMARK | 465 | | GLY C | 250 |
| REMARK | 465 | | GLY C | 251 |
| REMARK | 465 | | GLN C | 252 |
| REMARK | 465 | | TYR C | 253 |
| REMARK | 465 | | SER C | 265 |
| REMARK | 465 | | LEU C | 266 |
| REMARK | 465 | | GLU C | 275 |

| | | | | |
|---|---|---|---|---|
| SEQRES | 1 | A | 273 | VAL SER PRO ASN TYR ASP LYS TRP GLU MET GLU ARG THR |
| SEQRES | 2 | A | 273 | ASP ILE THR MET LYS HIS LYS LEU GLY GLY GLY GLN TYR |
| SEQRES | 3 | A | 273 | GLY GLU VAL TYR GLU GLY VAL TRP LYS LYS TYR SER LEU |
| SEQRES | 4 | A | 273 | THR VAL ALA VAL LYS THR LEU LYS GLU ASP THR MET GLU |
| SEQRES | 5 | A | 273 | VAL GLU GLU PHE LEU LYS GLU ALA ALA VAL MET LYS GLU |
| SEQRES | 6 | A | 273 | ILE LYS HIS PRO ASN LEU VAL GLN LEU LEU GLY VAL CYS |
| SEQRES | 7 | A | 273 | THR ARG GLU PRO PRO PHE TYR ILE ILE THR GLU PHE MET |
| SEQRES | 8 | A | 273 | THR TYR GLY ASN LEU LEU ASP TYR LEU ARG GLU CYS ASN |
| SEQRES | 9 | A | 273 | ARG GLN GLU VAL ASN ALA VAL VAL LEU LEU TYR MET ALA |
| SEQRES | 10 | A | 273 | THR GLN ILE SER SER ALA MET GLU TYR LEU GLU LYS LYS |
| SEQRES | 11 | A | 273 | ASN PHE ILE HIS ARG ASP LEU ALA ALA ARG ASN CYS LEU |
| SEQRES | 12 | A | 273 | VAL GLY GLU ASN HIS LEU VAL LYS VAL ALA ASP PHE GLY |
| SEQRES | 13 | A | 273 | LEU SER ARG LEU MET THR GLY ASP THR TYR THR ALA HIS |
| SEQRES | 14 | A | 273 | ALA GLY ALA LYS PHE PRO ILE LYS TRP THR ALA PRO GLU |
| SEQRES | 15 | A | 273 | SER LEU ALA TYR ASN LYS PHE SER ILE LYS SER ASP VAL |
| SEQRES | 16 | A | 273 | TRP ALA PHE GLY VAL LEU LEU TRP GLU ILE ALA TER TYR |
| SEQRES | 17 | A | 273 | GLY MET SER PRO TYR PRO GLY ILE ASP LEU SER GLN VAL |
| SEQRES | 18 | A | 273 | TYR GLU LEU LEU GLU LYS ASP TYR ARG MET GLU ARG PRO |
| SEQRES | 19 | A | 273 | GLU GLY CYS PRO GLU LYS VAL TYR GLU LEU MET ARG ALA |
| SEQRES | 20 | A | 273 | CYS TRP GLN TRP ASN PRO SER ASP ARG PRO SER PHE ALA |
| SEQRES | 21 | A | 273 | GLU ILE HIS GLN ALA PHE GLU THR MET PHE GLN GLU SER |
| SEQRES | 22 | B | 273 | VAL SER PRO ASN TYR ASP LYS TRP GLU MET GLU ARG THR |
| SEQRES | 23 | B | 273 | ASP ILE THR MET LYS HIS LYS LEU GLY GLY GLY GLN TYR |
| SEQRES | 24 | B | 273 | GLY GLU VAL TYR GLU GLY VAL TRP LYS LYS TYR SER LEU |
| SEQRES | 25 | B | 273 | THR VAL ALA VAL LYS THR LEU LYS GLU ASP THR MET GLU |
| SEQRES | 26 | B | 273 | VAL GLU GLU PHE LEU LYS GLU ALA ALA VAL MET LYS GLU |
| SEQRES | 27 | B | 273 | ILE LYS HIS PRO ASN LEU VAL GLN LEU LEU GLY VAL CYS |
| SEQRES | 28 | B | 273 | THR ARG GLU PRO PRO PHE TYR ILE ILE THR GLU PHE MET |
| SEQRES | 29 | B | 273 | THR TYR GLY ASN LEU LEU ASP TYR LEU ARG GLU CYS ASN |
| SEQRES | 30 | B | 273 | ARG GLN GLU VAL ASN ALA VAL VAL LEU LEU TYR MET ALA |
| SEQRES | 31 | B | 273 | THR GLN ILE SEE SER ALA MET GLU TYR LEU GLU LYS LYS |
| SEQRES | 32 | B | 273 | ASN PHE ILE HIS ARG ASP LEU ALA ALA ARG ASN CYS LEU |
| SEQRES | 33 | B | 273 | VAL GLY GLU ASN HIS LEU VAL LYS VAL ALA ASP PHE GLY |
| SEQRES | 34 | B | 273 | LEU SER ARG LEU MET THR GLY ASP THR TYR THR ALA HIS |
| SEQRES | 35 | B | 273 | ALA GLY ALA LYS PHE PRO ILE LYS TRP THR ALA PRO GLU |
| SEQRES | 36 | B | 273 | SER LEU ALA TYR ASN LYS PHE SER ILE LYS SER ASP VAL |
| SEQRES | 37 | B | 273 | TRP ALA PHE GLY VAL LEU LEU TRP GLU ILE ALA THR TYR |
| SEQRES | 38 | B | 273 | GLY MET SER PRO TYR PRO GLY ILE ASP LEU SER GLN VAL |
| SEQRES | 39 | B | 273 | TYR GLU LEU LEU GLU LYS ASP TYR ARG MET GLU ARG PRO |
| SEQRES | 40 | B | 273 | GLU GLY CYS PRO GLU LYS VAL TYR GLU LEU MET ARG ALA |
| SEQRES | 41 | B | 273 | CYS TRP GLN TRP ASN PRO SER ASP ARG PRO SER PHE ALA |
| SEQRES | 42 | B | 273 | GLU ILE HIS GLN ALA PHE GLU THR MET PHE GLN GLU SER |
| SEQRES | 43 | C | 273 | VAL SER PRO ASN TYR ASP LYS TRP GLU MET GLU ARG THR |
| SEQRES | 44 | C | 273 | ASP ILE THR MET LYS HIS LYS LEU GLY GLY GLY GLN TYR |
| SEQRES | 45 | C | 273 | GLY GLU VAL TYR GLU GLY VAL TRP LYS LYS TYR SER LEU |
| SEQRES | 46 | C | 273 | THR VAL ALA VAL LYS THR LEU LYS GLU ASP THR MET GLU |
| SEQRES | 47 | C | 273 | VAL GLU GLU PHE LEU LYS GLU ALA ALA VAL MET LYS GLU |
| SEQRES | 48 | C | 273 | ILE LYS HIS PRO ASN LEU VAL GLN LEU LEU GLY VAL CYS |
| SEQRES | 49 | C | 273 | THR ARG GLU PRO PRO PHE TYR ILE ILE THR GLU PHE MET |
| SEQRES | 50 | C | 273 | THR TYR GLY ASN LEU LEU ASP TYR LEU ARG GLU CYS ASN |
| SEQRES | 51 | C | 273 | ARG GLN GLU VAL ASN ALA VAL VAL LEU LEU TYR MET ALA |
| SEQRES | 52 | C | 273 | THR GLN ILE SER SER ALA MET GLU TYR LEU GLU LYS LYS |
| SEQRES | 53 | C | 273 | ASN PHE ILE HIS ARG ASP LEU ALA ALA ARG ASN CYS LEU |
| SEQRES | 54 | C | 273 | VAL GLY GLU ASN HIS LEU VAL LYS VAL ALA ASP PHE GLY |
| SEQRES | 55 | C | 273 | LEU SER ARG LEU MET THR GLY ASP THR TYR THR ALA HIS |
| SEQRES | 56 | C | 273 | ALA GLY ALA LYS PHE PRO ILE LYS TRP THR ALA PRO GLU |
| SEQRES | 57 | C | 273 | SER LEU ALA TYR ASN LYS PHE SER ILE LYS SER ASP VAL |
| SEQRES | 58 | C | 273 | TRP ALA PHE GLY VAL LEU LEU TRP GLU ILE ALA THR TYR |
| SEQRES | 59 | C | 273 | GLY MET SER PRO TYR PRO GLY ILE ASP LEU SER GLN VAL |
| SEQRES | 60 | C | 273 | TYR GLU LEU LEU GLU LYS ASP TYR ARG MET GLU ARG PRO |
| SEQRES | 61 | C | 273 | GLU GLY CYS PRO GLU LYS VAL TYR GLU LEU MET ARG ALA |
| SEQRES | 62 | C | 273 | CYS TRP GLN TRP ASN PRO SER ASP ARG PRO SER PHE ALA |
| SEQRES | 63 | C | 273 GLU ILE HIS GLN ALA PHE GLU THR MET PHE GLN GLU SER | |

| | | | | | | | |
|---|---|---|---|---|---|---|---|
| CRYST1 | 185.447 | 58.853 | 103.977 | 90.00 | 119.02 | 90.00 | C 1 2 1 |

| | | | | |
|---|---|---|---|---|
| SCALE1 | 0.005392 | 0.000000 | 0.002991 | 0.00000 |
| SCALE2 | 0.000000 | 0.016991 | 0.000000 | 0.00000 |
| SCALE3 | 0.000000 | 0.000000 | 0.010998 | 0.00000 |

| | | | | | | | | | | | | |
|---|---|---|---|---|---|---|---|---|---|---|---|---|
| ATOM | 1 | N | LYS | A | 234 | 26.818 | 13.365 | 34.898 | 1.00 | 78.16 | N | |
| ATOM | 2 | CA | LYS | A | 234 | 27.472 | 12.325 | 35.718 | 1.00 | 78.53 | C | |
| ATOM | 4 | CB | LYS | A | 234 | 26.778 | 12.076 | 37.074 | 1.00 | 78.69 | C | |
| ATOM | 7 | CG | LYS | A | 234 | 26.818 | 13.199 | 38.038 | 1.00 | 79.58 | C | |
| ATOM | 10 | CD | LYS | A | 234 | 28.206 | 13.505 | 38.576 | 1.00 | 80.52 | C | |
| ATOM | 13 | CE | LYS | A | 234 | 28.126 | 14.765 | 39.474 | 1.00 | 80.97 | C | |
| ATOM | 16 | NZ | LYS | A | 234 | 29.426 | 15.471 | 39.604 | 1.00 | 81.71 | N | |
| ATOM | 20 | C | LYS | A | 234 | 27.549 | 10.992 | 35.015 | 1.00 | 78.86 | C | |
| ATOM | 21 | O | LYS | A | 234 | 26.839 | 10.726 | 34.043 | 1.00 | 78.12 | O | |
| ATOM | 25 | N | TRP | A | 235 | 28.450 | 10.190 | 35.584 | 1.00 | 79.48 | N | |
| ATOM | 26 | CA | TRP | A | 235 | 28.766 | 8.811 | 35.242 | 1.00 | 79.74 | C | |
| ATOM | 28 | CB | TRP | A | 235 | 30.283 | 8.615 | 35.291 | 1.00 | 80.07 | C | |
| ATOM | 31 | CG | TRP | A | 235 | 31.057 | 9.632 | 34.521 | 1.00 | 80.26 | C | |
| ATOM | 32 | CD1 | TRP | A | 235 | 31.312 | 10.931 | 34.883 | 1.00 | 80.10 | C | |
| ATOM | 34 | NE1 | TRP | A | 235 | 32.055 | 11.554 | 33.911 | 1.00 | 80.57 | N | |
| ATOM | 36 | CE2 | TRP | A | 235 | 32.303 | 10.660 | 32.902 | 1.00 | 80.38 | C | |
| ATOM | 37 | CD2 | TRP | A | 235 | 31.684 | 9.435 | 33.259 | 1.00 | 80.12 | C | |
| ATOM | 38 | CE3 | TRP | A | 235 | 31.787 | 8.346 | 32.380 | 1.00 | 80.41 | C | |
| ATOM | 40 | CZ3 | TRP | A | 235 | 32.502 | 8.511 | 31.189 | 1.00 | 80.75 | C | |
| ATOM | 42 | CH2 | TRP | A | 235 | 33.102 | 9.747 | 30.864 | 1.00 | 80.77 | C | |
| ATOM | 44 | CZ2 | TRP | A | 235 | 33.021 | 10.826 | 31.709 | 1.00 | 80.35 | C | |
| ATOM | 46 | C | TRP | A | 235 | 28.160 | 7.868 | 36.259 | 1.00 | 79.87 | C | |
| ATOM | 47 | O | TRP | A | 235 | 28.013 | 6.678 | 36.000 | 1.00 | 79.41 | O | |
| ATOM | 49 | N | GLU | A | 236 | 27.923 | 8.397 | 37.458 | 1.00 | 80.54 | N | |
| ATOM | 50 | CA | GLU | A | 236 | 27.007 | 7.819 | 38.431 | 1.00 | 81.08 | C | |
| ATOM | 52 | CB | GLU | A | 236 | 26.608 | 8.856 | 39.505 | 1.00 | 81.08 | C | |
| ATOM | 55 | CG | GLU | A | 236 | 27.618 | 8.953 | 40.647 | 1.00 | 81.29 | C | |
| ATOM | 58 | CD | GLU | A | 236 | 27.762 | 7.660 | 41.444 | 1.00 | 80.91 | C | |
| ATOM | 59 | OE1 | GLU | A | 236 | 26.846 | 6.802 | 41.428 | 1.00 | 81.58 | O | |
| ATOM | 60 | OE2 | GLU | A | 236 | 28.804 | 7.480 | 42.089 | 1.00 | 79.08 | O | |
| ATOM | 61 | C | GLU | A | 236 | 25.755 | 7.204 | 37.820 | 1.00 | 81.19 | C | |
| ATOM | 62 | O | GLU | A | 236 | 25.034 | 7.862 | 37.062 | 1.00 | 81.13 | O | |
| ATOM | 64 | N | MET | A | 237 | 25.546 | 5.933 | 38.156 | 1.00 | 81.30 | N | |
| ATOM | 65 | CA | MET | A | 237 | 24.476 | 5.131 | 37.602 | 1.00 | 81.81 | C | |
| ATOM | 67 | CB | MET | A | 237 | 24.896 | 4.640 | 36.209 | 1.00 | 81.98 | C | |
| ATOM | 70 | CG | MET | A | 237 | 26.044 | 3.646 | 36.188 | 1.00 | 83.64 | C | |
| ATOM | 73 | SD | MET | A | 237 | 25.463 | 1.940 | 36.032 | 1.00 | 87.27 | S | |
| ATOM | 74 | CE | MET | A | 237 | 25.986 | 1.190 | 37.497 | 1.00 | 86.41 | C | |
| ATOM | 78 | C | MET | A | 237 | 24.082 | 3.993 | 38.579 | 1.00 | 81.67 | C | |
| ATOM | 79 | O | MET | A | 237 | 24.632 | 3.891 | 39.683 | 1.00 | 81.67 | O | |
| ATOM | 81 | N | GLU | A | 238 | 23.140 | 3.150 | 38.163 | 1.00 | 81.49 | N | |
| ATOM | 82 | CA | GLU | A | 238 | 22.421 | 2.269 | 39.068 | 1.00 | 81.47 | C | |
| ATOM | 84 | CB | GLU | A | 238 | 20.909 | 2.315 | 38.738 | 1.00 | 81.55 | C | |
| ATOM | 87 | CG | GLU | A | 238 | 19.998 | 1.412 | 39.562 | 1.00 | 81.65 | C | |
| ATOM | 90 | CD | GLU | A | 238 | 20.276 | 1.457 | 41.059 | 1.00 | 81.86 | C | |
| ATOM | 91 | OE1 | GLU | A | 238 | 19.951 | 2.470 | 41.713 | 1.00 | 83.01 | O | |
| ATOM | 92 | OE2 | GLU | A | 238 | 20.831 | 0.480 | 41.580 | 1.00 | 80.92 | O | |
| ATOM | 93 | C | GLU | A | 238 | 22.985 | 0.840 | 39.030 | 1.00 | 81.31 | C | |
| ATOM | 94 | O | GLU | A | 238 | 22.810 | 0.112 | 38.048 | 1.00 | 81.40 | O | |
| ATOM | 96 | N | ARG | A | 239 | 23.703 | 0.496 | 40.102 | 1.00 | 81.18 | N | |
| ATOM | 97 | CA | ARG | A | 239 | 24.079 | −0.872 | 40.513 | 1.00 | 81.06 | C | |
| ATOM | 99 | CB | ARG | A | 239 | 23.987 | −0.932 | 42.037 | 1.00 | 81.08 | C | |
| ATOM | 102 | CG | ARG | A | 239 | 24.233 | −2.292 | 42.715 | 1.00 | 80.93 | C | |
| ATOM | 105 | CD | ARG | A | 239 | 24.461 | −2.217 | 44.238 | 1.00 | 80.06 | C | |
| ATOM | 108 | NE | ARG | A | 239 | 25.195 | −1.006 | 44.639 | 1.00 | 79.61 | N | |
| ATOM | 110 | CZ | ARG | A | 239 | 26.489 | −0.735 | 44.376 | 1.00 | 77.88 | C | |
| ATOM | 111 | NH1 | ARG | A | 239 | 27.255 | −1.600 | 43.701 | 1.00 | 76.95 | N | |
| ATOM | 114 | NH2 | ARG | A | 239 | 27.006 | 0.427 | 44.790 | 1.00 | 76.22 | N | |
| ATOM | 117 | C | ARG | A | 239 | 23.245 | −2.005 | 39.940 | 1.00 | 81.74 | C | |
| ATOM | 118 | O | ARG | A | 239 | 23.794 | −2.917 | 39.320 | 1.00 | 82.14 | O | |
| ATOM | 120 | N | THR | A | 240 | 21.926 | −1.917 | 40.158 | 1.00 | 82.28 | N | |
| ATOM | 121 | CA | THR | A | 240 | 20.937 | −2.963 | 39.827 | 1.00 | 82.51 | C | |
| ATOM | 123 | CB | THR | A | 240 | 19.536 | −2.506 | 40.239 | 1.00 | 82.46 | C | |
| ATOM | 125 | OG1 | THR | A | 240 | 19.579 | −1.976 | 41.558 | 1.00 | 81.86 | O | |
| ATOM | 127 | CG2 | THR | A | 240 | 18.553 | −3.670 | 40.339 | 1.00 | 82.61 | C | |
| ATOM | 131 | C | THR | A | 240 | 20.877 | −3.336 | 38.363 | 1.00 | 83.25 | C | |
| ATOM | 132 | O | THR | A | 240 | 20.510 | −4.462 | 38.049 | 1.00 | 83.29 | O | |
| ATOM | 134 | N | ASP | A | 241 | 21.243 | −2.409 | 37.472 | 1.00 | 84.25 | N | |
| ATOM | 135 | CA | ASP | A | 241 | 21.297 | −2.700 | 36.021 | 1.00 | 85.16 | C | |
| ATOM | 137 | CB | ASP | A | 241 | 21.595 | −1.436 | 35.172 | 1.00 | 85.09 | C | |
| ATOM | 140 | CG | ASP | A | 241 | 20.594 | −0.286 | 35.372 | 1.00 | 85.12 | C | |
| ATOM | 141 | OD1 | ASP | A | 241 | 19.460 | −0.512 | 35.871 | 1.00 | 85.91 | O | |
| ATOM | 142 | OD2 | ASP | A | 241 | 20.880 | 0.892 | 35.024 | 1.00 | 82.69 | O | |
| ATOM | 143 | C | ASP | A | 241 | 22.322 | −3.802 | 35.604 | 1.00 | 85.66 | C | |
| ATOM | 144 | O | ASP | A | 241 | 22.244 | −4.287 | 34.467 | 1.00 | 86.40 | O | |
| ATOM | 146 | N | ILE | A | 242 | 23.245 | −4.203 | 36.491 | 1.00 | 85.57 | N | |
| ATOM | 147 | CA | ILE | A | 242 | 24.346 | −5.096 | 36.135 | 1.00 | 85.67 | C | |
| ATOM | 149 | CB | ILE | A | 242 | 25.684 | −4.390 | 36.511 | 1.00 | 86.01 | C | |
| ATOM | 151 | CG1 | ILE | A | 242 | 25.948 | −3.260 | 35.500 | 1.00 | 86.69 | C | |
| ATOM | 154 | CD1 | ILE | A | 242 | 26.758 | −2.134 | 36.054 | 1.00 | 87.46 | C | |
| ATOM | 158 | CG2 | ILE | A | 242 | 26.911 | −5.364 | 36.561 | 1.00 | 85.74 | C | |
| ATOM | 162 | C | ILE | A | 242 | 24.226 | −6.496 | 36.747 | 1.00 | 85.61 | C | |
| ATOM | 163 | O | ILE | A | 242 | 24.393 | −6.660 | 37.946 | 1.00 | 85.93 | O | |
| ATOM | 165 | N | THR | A | 243 | 23.963 | −7.501 | 35.915 | 1.00 | 85.46 | N | |
| ATOM | 166 | CA | THR | A | 243 | 24.156 | −8.909 | 36.309 | 1.00 | 85.54 | C | |
| ATOM | 168 | CB | THR | A | 243 | 23.537 | −9.883 | 35.234 | 1.00 | 85.52 | C | |
| ATOM | 170 | OG1 | THR | A | 243 | 22.120 | −9.986 | 35.422 | 1.00 | 84.66 | O | |
| ATOM | 172 | CG2 | THR | A | 243 | 24.031 | −11.347 | 35.374 | 1.00 | 85.36 | C | |
| ATOM | 176 | C | THR | A | 243 | 25.656 | −9.140 | 36.424 | 1.00 | 85.52 | C | |
| ATOM | 177 | O | THR | A | 243 | 26.337 | −8.984 | 35.427 | 1.00 | 85.69 | O | |
| ATOM | 179 | N | MET | A | 244 | 26.167 | −9.499 | 37.603 | 1.00 | 85.53 | N | |
| ATOM | 180 | CA | MET | A | 244 | 27.580 | −9.934 | 37.728 | 1.00 | 85.86 | C | |
| ATOM | 182 | CB | MET | A | 244 | 28.085 | −9.803 | 39.170 | 1.00 | 85.70 | C | |
| ATOM | 185 | CG | MET | A | 244 | 28.112 | −8.375 | 39.709 | 1.00 | 85.54 | C | |
| ATOM | 188 | SD | MET | A | 244 | 29.385 | −7.266 | 39.012 | 1.00 | 86.87 | S | |
| ATOM | 189 | CE | MET | A | 244 | 30.875 | −7.696 | 40.018 | 1.00 | 87.07 | C | |
| ATOM | 193 | C | MET | A | 244 | 27.782 | −11.381 | 37.206 | 1.00 | 86.18 | C | |
| ATOM | 194 | O | MET | A | 244 | 26.801 | −12.120 | 37.033 | 1.00 | 86.44 | O | |
| ATOM | 196 | N | LYS | A | 245 | 29.043 | −11.763 | 36.937 | 1.00 | 86.44 | N | |
| ATOM | 197 | CA | LYS | A | 245 | 29.409 | −13.137 | 36.496 | 1.00 | 86.63 | C | |
| ATOM | 199 | CB | LYS | A | 245 | 29.563 | −13.206 | 34.970 | 1.00 | 86.76 | C | |
| ATOM | 202 | CG | LYS | A | 245 | 28.215 | −13.142 | 34.218 | 1.00 | 86.71 | C | |
| ATOM | 205 | CD | LYS | A | 245 | 28.247 | −13.856 | 32.849 | 1.00 | 86.59 | C | |
| ATOM | 208 | CE | LYS | A | 245 | 26.887 | −14.432 | 32.489 | 1.00 | 86.97 | C | |
| ATOM | 211 | NZ | LYS | A | 245 | 25.759 | −13.459 | 32.726 | 1.00 | 87.93 | N | |
| ATOM | 215 | C | LYS | A | 245 | 30.615 | −13.792 | 37.219 | 1.00 | 86.82 | C | |
| ATOM | 216 | O | LYS | A | 245 | 30.406 | −14.733 | 37.985 | 1.00 | 87.03 | O | |
| ATOM | 218 | N | HIS | A | 246 | 31.846 | −13.333 | 36.991 | 1.00 | 86.96 | N | |
| ATOM | 219 | CA | HIS | A | 246 | 33.037 | −14.010 | 37.569 | 1.00 | 87.20 | C | |
| ATOM | 221 | CB | HIS | A | 246 | 33.264 | −15.363 | 36.865 | 1.00 | 87.43 | C | |
| ATOM | 224 | CG | HIS | A | 246 | 33.300 | −15.259 | 35.370 | 1.00 | 87.88 | C | |
| ATOM | 225 | ND1 | HIS | A | 246 | 34.372 | −14.723 | 34.685 | 1.00 | 87.72 | N | |
| ATOM | 227 | CE1 | HIS | A | 246 | 34.122 | −14.751 | 33.390 | 1.00 | 87.65 | C | |
| ATOM | 229 | NE2 | HIS | A | 246 | 32.921 | −15.274 | 33.209 | 1.00 | 88.08 | N | |
| ATOM | 231 | CD2 | HIS | A | 246 | 32.385 | −15.598 | 34.431 | 1.00 | 88.07 | C | |
| ATOM | 233 | C | HIS | A | 246 | 34.328 | −13.205 | 37.466 | 1.00 | 87.24 | C | |
| ATOM | 234 | O | HIS | A | 246 | 34.415 | −12.279 | 36.659 | 1.00 | 87.14 | O | |
| ATOM | 236 | N | LYS | A | 247 | 35.343 | −13.649 | 38.217 | 1.00 | 87.30 | N | |
| ATOM | 237 | CA | LYS | A | 247 | 36.687 | −13.015 | 38.257 | 1.00 | 87.60 | C | |
| ATOM | 239 | CB | LYS | A | 247 | 37.676 | −13.739 | 39.200 | 1.00 | 87.74 | C | |
| ATOM | 242 | CG | LYS | A | 247 | 37.220 | −13.966 | 40.654 | 1.00 | 87.39 | C | |
| ATOM | 245 | CD | LYS | A | 247 | 37.494 | −15.411 | 41.116 | 1.00 | 86.50 | C | |
| ATOM | 248 | CE | LYS | A | 247 | 36.404 | −15.928 | 42.014 | 1.00 | 86.08 | C | |
| ATOM | 251 | NZ | LYS | A | 247 | 36.583 | −17.383 | 42.190 | 1.00 | 84.97 | N | |
| ATOM | 255 | C | LYS | A | 247 | 37.367 | −12.956 | 36.895 | 1.00 | 87.89 | C | |
| ATOM | 256 | O | LYS | A | 247 | 37.052 | −13.741 | 36.006 | 1.00 | 87.86 | O | |
| ATOM | 258 | N | LEU | A | 248 | 38.342 | −12.04 | 8 36.791 | 1.00 | 88.30 | N | |
| ATOM | 259 | CA | LEU | A | 248 | 38.956 | −11.66 | 3 35.524 | 1.00 | 88.54 | C | |
| ATOM | 261 | CB | LEU | A | 248 | 38.469 | −10.259 | 35.116 | 1.00 | 88.42 | C | |
| ATOM | 264 | CG | LEU | A | 248 | 38.095 | −10.118 | 33.629 | 1.00 | 89.36 | C | |
| ATOM | 266 | CD1 | LEU | A | 248 | 36.673 | −10.649 | 33.379 | 1.00 | 88.25 | C | |
| ATOM | 270 | CD2 | LEU | A | 248 | 38.272 | −8.655 | 33.057 | 1.00 | 89.70 | C | |
| ATOM | 274 | C | LEU | A | 248 | 40.470 | −11.714 | 35.658 | 1.00 | 88.12 | C | |
| ATOM | 275 | O | LEU | A | 248 | 41.055 | −12.791 | 35.545 | 1.00 | 87.99 | O | |
| ATOM | 277 | N | GLY | A | 254 | 42.071 | −4.427 | 44.620 | 1.00 | 88.95 | N | |
| ATOM | 278 | CA | GLY | A | 254 | 40.680 | −4.664 | 44.224 | 1.00 | 89.27 | C | |
| ATOM | 281 | C | GLY | A | 254 | 40.501 | −5.449 | 42.920 | 1.00 | 89.51 | C | |
| ATOM | 282 | O | GLY | A | 254 | 41.369 | −5.399 | 42.038 | 1.00 | 89.70 | O | |
| ATOM | 284 | N | GLU | A | 255 | 39.344 | −6.117 | 42.786 | 1.00 | 89.44 | N | |
| ATOM | 285 | CA | GLU | A | 255 | 39.090 | −7.168 | 41.755 | 1.00 | 88.99 | C | |
| ATOM | 287 | CB | GLU | A | 255 | 38.439 | −8.396 | 42.415 | 1.00 | 88.93 | C | |
| ATOM | 290 | CG | GLU | A | 255 | 39.408 | −9.201 | 43.283 | 1.00 | 89.57 | C | |
| ATOM | 293 | CD | GLU | A | 255 | 38.744 | −10.069 | 44.357 | 1.00 | 89.88 | C | |
| ATOM | 294 | OE1 | GLU | A | 255 | 37.533 | −9.895 | 44.674 | 1.00 | 90.32 | O | |
| ATOM | 295 | OE2 | GLU | A | 255 | 39.460 | −10.939 | 44.907 | 1.00 | 90.32 | O | |
| ATOM | 296 | C | GLU | A | 255 | 38.210 | −6.715 | 40.579 | 1.00 | 88.56 | C | |
| ATOM | 297 | O | GLU | A | 255 | 37.374 | −5.838 | 40.744 | 1.00 | 89.00 | O | |
| ATOM | 299 | N | VAL | A | 256 | 38.396 | −7.364 | 39.423 | 1.00 | 87.76 | N | |
| ATOM | 300 | CA | VAL | A | 256 | 37.652 | −7.123 | 38.175 | 1.00 | 87.33 | C | |
| ATOM | 302 | CB | VAL | A | 256 | 38.621 | −6.876 | 36.959 | 1.00 | 87.32 | C | |
| ATOM | 304 | CG1 | VAL | A | 256 | 37.863 | −6.540 | 35.667 | 1.00 | 87.04 | C | |
| ATOM | 308 | CG2 | VAL | A | 256 | 39.615 | −5.743 | 37.249 | 1.00 | 86.86 | C | |
| ATOM | 312 | C | VAL | A | 256 | 36.752 | −8.334 | 37.881 | 1.00 | 86.63 | C | |
| ATOM | 313 | O | VAL | A | 256 | 37.148 | −9.481 | 38.100 | 1.00 | 86.42 | O | |
| ATOM | 315 | N | TYR | A | 257 | 35.541 | −8.060 | 37.392 | 1.00 | 85.89 | N | |
| ATOM | 316 | CA | TYR | A | 257 | 34.536 | −9.087 | 37.115 | 1.00 | 85.28 | C | |
| ATOM | 318 | CB | TYR | A | 257 | 33.411 | −9.068 | 38.168 | 1.00 | 85.83 | C | |
| ATOM | 321 | CG | TYR | A | 257 | 33.876 | −9.525 | 39.535 | 1.00 | 86.28 | C | |
| ATOM | 322 | CD1 | TYR | A | 257 | 34.611 | −8.663 | 40.364 | 1.00 | 86.91 | C | |
| ATOM | 324 | CE1 | TYR | A | 257 | 35.091 | −9.076 | 41.611 | 1.00 | 87.58 | C | |
| ATOM | 326 | CZ | TYR | A | 257 | 34.823 | −10.362 | 42.064 | 1.00 | 87.23 | C | |
| ATOM | 327 | OH | TYR | A | 257 | 35.297 | −10.728 | 43.303 | 1.00 | 86.48 | O | |
| ATOM | 329 | CE2 | TYR | A | 257 | 34.085 | −11.248 | 41.263 | 1.00 | 86.98 | C | |
| ATOM | 331 | CD2 | TYR | A | 257 | 33.617 | −10.821 | 39.994 | 1.00 | 86.43 | C | |
| ATOM | 333 | C | TYR | A | 257 | 33.940 | −8.833 | 35.766 | 1.00 | 84.40 | C | |
| ATOM | 334 | O | TYR | A | 257 | 33.750 | −7.674 | 35.395 | 1.00 | 84.03 | O | |
| ATOM | 336 | N | GLU | A | 258 | 33.634 | −9.906 | 35.034 | 1.00 | 83.48 | N | |
| ATOM | 337 | CA | GLU | A | 258 | 32.751 | −9.785 | 33.867 | 1.00 | 82.83 | C | |
| ATOM | 339 | CB | GLU | A | 258 | 32.700 | −11.069 | 33.033 | 1.00 | 82.76 | C | |
| ATOM | 342 | CG | GLU | A | 258 | 32.002 | −10.883 | 31.689 | 1.00 | 83.06 | C | |
| ATOM | 345 | CD | GLU | A | 258 | 31.950 | −12.151 | 30.854 | 1.00 | 83.34 | C | |
| ATOM | 346 | OE1 | GLU | A | 258 | 32.996 | −12.810 | 30.663 | 1.00 | 83.83 | O | |
| ATOM | 347 | OE2 | GLU | A | 258 | 30.856 | −12.489 | 30.358 | 1.00 | 85.42 | O | |
| ATOM | 348 | C | GLU | A | 258 | 31.345 | −9.428 | 34.381 | 1.00 | 82.52 | C | |
| ATOM | 349 | O | GLU | A | 258 | 30.874 | −10.022 | 35.357 | 1.00 | 82.83 | O | |
| ATOM | 351 | N | GLY | A | 259 | 30.712 | −8.445 | 33.737 | 1.00 | 81.70 | N | |
| ATOM | 352 | CA | GLY | A | 259 | 29.334 | −8.023 | 34.026 | 1.00 | 80.99 | C | |
| ATOM | 355 | C | GLY | A | 259 | 28.494 | −7.888 | 32.756 | 1.00 | 79.87 | C | |
| ATOM | 356 | O | GLY | A | 259 | 29.026 | −7.932 | 31.638 | 1.00 | 79.33 | O | |
| ATOM | 358 | N | VAL | A | 260 | 27.173 | −7.756 | 32.955 | 1.00 | 78.85 | N | |
| ATOM | 359 | CA | VAL | A | 260 | 26.187 | −7.545 | 31.884 | 1.00 | 77.72 | C | |
| ATOM | 361 | CB | VAL | A | 260 | 25.367 | −8.820 | 31.548 | 1.00 | 77.27 | C | |
| ATOM | 363 | CG1 | VAL | A | 260 | 24.669 | −8.654 | 30.210 | 1.00 | 76.96 | C | |
| ATOM | 367 | CG2 | VAL | A | 260 | 26.243 | −10.045 | 31.472 | 1.00 | 77.81 | C | |
| ATOM | 371 | C | VAL | A | 260 | 25.234 | −6.401 | 32.272 | 1.00 | 77.02 | C | |
| ATOM | 372 | O | VAL | A | 260 | 24.499 | −6.525 | 33.253 | 1.00 | 76.86 | O | |
| ATOM | 374 | N | TRP | A | 261 | 25.257 | −5.313 | 31.493 | 1.00 | 76.10 | N | |
| ATOM | 375 | CA | TRP | A | 261 | 24.374 | −4.174 | 31.686 | 1.00 | 75.92 | C | |
| ATOM | 377 | CB | TRP | A | 261 | 25.015 | −2.881 | 31.122 | 1.00 | 75.41 | C | |
| ATOM | 380 | CG | TRP | A | 261 | 24.434 | −1.571 | 31.658 | 1.00 | 75.36 | C | |
| ATOM | 381 | CD1 | TRP | A | 261 | 23.142 | −1.352 | 32.059 | 1.00 | 75.54 | C | |
| ATOM | 383 | NE1 | TRP | A | 261 | 22.975 | −0.049 | 32.472 | 1.00 | 75.59 | N | |
| ATOM | 385 | CE2 | TRP | A | 261 | 24.158 | 0.626 | 32.328 | 1.00 | 74.96 | C | |
| ATOM | 386 | CD2 | TRP | A | 261 | 25.107 | −0.296 | 31.810 | 1.00 | 75.43 | C | |
| ATOM | 387 | CE3 | TRP | A | 261 | 26.422 | 0.156 | 31.592 | 1.00 | 74.99 | C | |
| ATOM | 389 | CZ3 | TRP | A | 261 | 26.738 | 1.512 | 31.884 | 1.00 | 74.65 | C | |
| ATOM | 391 | CH2 | TRP | A | 261 | 25.759 | 2.393 | 32.383 | 1.00 | 74.88 | C | |
| ATOM | 393 | CZ2 | TRP | A | 261 | 24.473 | 1.973 | 32.614 | 1.00 | 74.58 | C | |
| ATOM | 395 | C | TRP | A | 261 | 23.016 | −4.568 | 31.026 | 1.00 | 76.27 | C | |
| ATOM | 396 | O | TRP | A | 261 | 22.785 | −4.309 | 29.835 | 1.00 | 76.64 | O | |
| ATOM | 398 | N | LYS | A | 262 | 22.156 | −5.227 | 31.827 | 1.00 | 76.14 | N | |
| ATOM | 399 | CA | LYS | A | 262 | 20.798 | −5.733 | 31.448 | 1.00 | 75.87 | C | |
| ATOM | 401 | CB | LYS | A | 262 | 19.889 | −5.798 | 32.700 | 1.00 | 75.99 | C | |
| ATOM | 404 | CG | LYS | A | 262 | 20.218 | −6.892 | 33.729 | 1.00 | 75.98 | C | |
| ATOM | 407 | CD | LYS | A | 262 | 19.696 | −6.545 | 35.141 | 1.00 | 75.65 | C | |
| ATOM | 410 | CE | LYS | A | 262 | 20.231 | −7.530 | 36.181 | 1.00 | 75.98 | C | |
| ATOM | 413 | NZ | LYS | A | 262 | 19.984 | −7.153 | 37.603 | 1.00 | 75.85 | N | |
| ATOM | 417 | C | LYS | A | 262 | 20.056 | −4.909 | 30.379 | 1.00 | 75.79 | C | |
| ATOM | 418 | O | LYS | A | 262 | 19.639 | −5.440 | 29.336 | 1.00 | 75.56 | O | |
| ATOM | 420 | N | LYS | A | 263 | 19.927 | −3.613 | 30.683 | 1.00 | 75.60 | N | |
| ATOM | 421 | CA | LYS | A | 263 | 19.263 | −2.569 | 29.857 | 1.00 | 75.28 | C | |
| ATOM | 423 | CB | LYS | A | 263 | 19.536 | −1.201 | 30.545 | 1.00 | 75.29 | C | |
| ATOM | 426 | CG | LYS | A | 263 | 19.048 | 0.072 | 29.892 | 1.00 | 75.41 | C | |
| ATOM | 429 | CD | LYS | A | 263 | 19.257 | 1.300 | 30.841 | 1.00 | 74.82 | C | |
| ATOM | 432 | CE | LYS | A | 263 | 18.101 | 1.534 | 31.815 | 1.00 | 74.33 | C | |
| ATOM | 435 | NZ | LYS | A | 263 | 17.792 | 2.983 | 32.047 | 1.00 | 72.65 | N | |
| ATOM | 439 | C | LYS | A | 263 | 19.676 | −2.563 | 28.363 | 1.00 | 75.05 | C | |
| ATOM | 440 | O | LYS | A | 263 | 18.827 | −2.439 | 27.489 | 1.00 | 74.92 | O | |
| ATOM | 442 | N | TYR | A | 264 | 20.972 | −2.757 | 28.098 | 1.00 | 75.05 | N | |
| ATOM | 443 | CA | TYR | A | 264 | 21.581 | −2.629 | 26.759 | 1.00 | 74.80 | C | |
| ATOM | 445 | CB | TYR | A | 264 | 22.679 | −1.564 | 26.808 | 1.00 | 74.92 | C | |
| ATOM | 448 | CG | TYR | A | 264 | 22.337 | −0.270 | 27.497 | 1.00 | 74.94 | C | |
| ATOM | 449 | CD1 | TYR | A | 264 | 22.498 | −0.123 | 28.867 | 1.00 | 75.62 | C | |
| ATOM | 451 | CE1 | TYR | A | 264 | 22.220 | 1.096 | 29.503 | 1.00 | 75.76 | C | |
| ATOM | 453 | CZ | TYR | A | 264 | 21.798 | 2.176 | 28.771 | 1.00 | 75.40 | C | |
| ATOM | 454 | OH | TYR | A | 264 | 21.539 | 3.345 | 29.430 | 1.00 | 75.16 | O | |
| ATOM | 456 | CE2 | TYR | A | 264 | 21.657 | 2.070 | 27.402 | 1.00 | 75.92 | C | |
| ATOM | 458 | CD2 | TYR | A | 264 | 21.937 | 0.840 | 26.770 | 1.00 | 75.74 | C | |
| ATOM | 460 | C | TYR | A | 264 | 22.211 | −3.932 | 26.212 | 1.00 | 74.67 | C | |
| ATOM | 461 | O | TYR | A | 264 | 23.180 | −3.877 | 25.434 | 1.00 | 74.39 | O | |
| ATOM | 463 | N | SER | A | 265 | 21.673 | −5.087 | 26.627 | 1.00 | 74.64 | N | |
| ATOM | 464 | CA | SER | A | 265 | 22.172 | −6.457 | 26.279 | 1.00 | 74.05 | C | |
| ATOM | 466 | CB | SER | A | 265 | 21.518 | −6.904 | 24.950 | 1.00 | 74.19 | C | |
| ATOM | 469 | OG | SER | A | 265 | 21.420 | −8.318 | 24.842 | 1.00 | 74.38 | O | |
| ATOM | 471 | C | SER | A | 265 | 23.721 | −6.661 | 26.262 | 1.00 | 73.43 | C | |
| ATOM | 472 | O | SER | A | 265 | 24.234 | −7.570 | 25.607 | 1.00 | 73.19 | O | |
| ATOM | 474 | N | LEU | A | 266 | 24.430 | −5.850 | 27.051 | 1.00 | 72.71 | N | |
| ATOM | 475 | CA | LEU | A | 266 | 25.854 | −5.506 | 26.815 | 1.00 | 72.45 | C | |
| ATOM | 477 | CB | LEU | A | 266 | 26.016 | −3.971 | 26.872 | 1.00 | 72.46 | C | |
| ATOM | 480 | CG | LEU | A | 266 | 27.398 | −3.319 | 26.983 | 1.00 | 72.21 | C | |
| ATOM | 482 | CD1 | LEU | A | 266 | 28.158 | −3.552 | 25.679 | 1.00 | 71.84 | C | |
| ATOM | 486 | CD2 | LEU | A | 266 | 27.239 | −1.836 | 27.293 | 1.00 | 72.67 | C | |
| ATOM | 490 | C | LEU | A | 266 | 26.793 | −6.096 | 27.849 | 1.00 | 71.24 | C | |
| ATOM | 491 | O | LEU | A | 266 | 26.587 | −5.832 | 29.031 | 1.00 | 71.07 | O | |
| ATOM | 493 | N | THR | A | 267 | 27.846 | −6.804 | 27.413 | 1.00 | 69.96 | N | |
| ATOM | 494 | CA | THR | A | 267 | 28.921 | −7.217 | 28.317 | 1.00 | 69.27 | C | |
| ATOM | 496 | CB | THR | A | 267 | 29.662 | −8.485 | 27.811 | 1.00 | 69.35 | C | |
| ATOM | 498 | OG1 | THR | A | 267 | 28.791 | −9.614 | 27.913 | 1.00 | 68.85 | O | |
| ATOM | 500 | CG2 | THR | A | 267 | 30.805 | −8.923 | 28.752 | 1.00 | 69.65 | C | |
| ATOM | 504 | C | THR | A | 267 | 29.892 | −6.051 | 28.623 | 1.00 | 69.12 | C | |
| ATOM | 505 | O | THR | A | 267 | 30.276 | −5.267 | 27.741 | 1.00 | 68.18 | O | |
| ATOM | 507 | N | VAL | A | 268 | 30.277 | −5.987 | 29.902 | 1.00 | 69.34 | N | |
| ATOM | 508 | CA | VAL | A | 268 | 31.144 | −4.951 | 30.489 | 1.00 | 69.17 | C | |
| ATOM | 510 | CB | VAL | A | 268 | 30.334 | −3.902 | 31.288 | 1.00 | 68.79 | C | |
| ATOM | 512 | CG1 | VAL | A | 268 | 29.660 | −2.936 | 30.364 | 1.00 | 68.30 | C | |
| ATOM | 516 | CG2 | VAL | A | 268 | 29.309 | −4.558 | 32.208 | 1.00 | 68.29 | C | |
| ATOM | 520 | C | VAL | A | 268 | 32.161 | −5.572 | 31.443 | 1.00 | 69.57 | C | |
| ATOM | 521 | O | VAL | A | 268 | 32.005 | −6.699 | 31.894 | 1.00 | 69.27 | O | |
| ATOM | 523 | N | ALA | A | 269 | 33.216 | −4.829 | 31.724 | 1.00 | 70.81 | N | |
| ATOM | 524 | CA | ALA | A | 269 | 34.108 | −5.141 | 32.849 | 1.00 | 71.81 | C | |
| ATOM | 526 | CB | ALA | A | 269 | 35.551 | −4.853 | 32.484 | 1.00 | 71.50 | C | |
| ATOM | 530 | C | ALA | A | 269 | 33.658 | −4.259 | 34.006 | 1.00 | 72.55 | C | |
| ATOM | 531 | O | ALA | A | 269 | 33.377 | −3.086 | 33.789 | 1.00 | 72.88 | O | |
| ATOM | 533 | N | VAL | A | 270 | 33.551 | −4.821 | 35.209 | 1.00 | 73.46 | N | |
| ATOM | 534 | CA | VAL | A | 270 | 33.347 | −4.030 | 36.420 | 1.00 | 74.63 | C | |
| ATOM | 536 | CB | VAL | A | 270 | 31.934 | −4.263 | 37.049 | 1.00 | 74.51 | C | |
| ATOM | 538 | CG1 | VAL | A | 270 | 31.677 | −3.297 | 38.215 | 1.00 | 74.25 | C | |
| ATOM | 542 | CG2 | VAL | A | 270 | 30.826 | −4.095 | 35.996 | 1.00 | 74.34 | C | |
| ATOM | 546 | C | VAL | A | 270 | 34.499 | −4.312 | 37.414 | 1.00 | 75.53 | C | |
| ATOM | 547 | O | VAL | A | 270 | 34.587 | −5.432 | 37.967 | 1.00 | 75.75 | O | |
| ATOM | 549 | N | LYS | A | 271 | 35.392 | −3.315 | 37.589 | 1.00 | 76.54 | N | |
| ATOM | 550 | CA | LYS | A | 271 | 36.379 | −3.289 | 38.691 | 1.00 | 76.71 | C | |
| ATOM | 552 | CB | LYS | A | 271 | 37.650 | −2.423 | 38.405 | 1.00 | 77.09 | C | |
| ATOM | 555 | CG | LYS | A | 271 | 38.873 | −2.742 | 39.415 | 1.00 | 77.54 | C | |
| ATOM | 558 | CD | LYS | A | 271 | 40.361 | −2.133 | 39.146 | 1.00 | 77.33 | C | |
| ATOM | 561 | CE | LYS | A | 271 | 41.168 | −1.921 | 40.537 | 1.00 | 76.92 | C | |
| ATOM | 564 | NZ | LYS | A | 271 | 42.674 | −2.166 | 40.628 | 1.00 | 74.71 | N | |
| ATOM | 568 | C | LYS | A | 271 | 35.653 | −2.833 | 39.955 | 1.00 | 77.26 | C | |
| ATOM | 569 | O | LYS | A | 271 | 35.034 | −1.781 | 39.974 | 1.00 | 76.34 | O | |
| ATOM | 571 | N | THR | A | 272 | 35.730 | −3.670 | 40.985 | 1.00 | 78.63 | N | |
| ATOM | 572 | CA | THR | A | 272 | 35.006 | −3.516 | 42.236 | 1.00 | 80.00 | C | |
| ATOM | 574 | CB | THR | A | 272 | 34.149 | −4.817 | 42.470 | 1.00 | 79.55 | C | |
| ATOM | 576 | OG1 | THR | A | 272 | 33.042 | −4.529 | 43.315 | 1.00 | 79.43 | O | |
| ATOM | 578 | CG2 | THR | A | 272 | 34.891 | −5.928 | 43.203 | 1.00 | 79.48 | C | |
| ATOM | 582 | C | THR | A | 272 | 35.991 | −3.196 | 43.382 | 1.00 | 80.67 | C | |
| ATOM | 583 | O | THR | A | 272 | 37.211 | −3.413 | 43.241 | 1.00 | 80.64 | O | |
| ATOM | 585 | N | LEU | A | 273 | 35.459 | −2.668 | 44.490 | 1.00 | 81.70 | N | |
| ATOM | 586 | CA | LEU | A | 273 | 36.216 | −2.502 | 45.754 | 1.00 | 82.45 | C | |
| ATOM | 588 | CB | LEU | A | 273 | 36.662 | −1.051 | 45.952 | 1.00 | 82.31 | C | |
| ATOM | 591 | CG | LEU | A | 273 | 37.804 | −0.832 | 46.971 | 1.00 | 81.89 | C | |
| ATOM | 593 | CD1 | LEU | A | 273 | 39.194 | −1.166 | 46.387 | 1.00 | 81.15 | C | |
| ATOM | 597 | CD2 | LEU | A | 273 | 37.793 | 0.592 | 47.501 | 1.00 | 81.96 | C | |
| ATOM | 601 | C | LEU | A | 273 | 35.398 | −2.958 | 46.968 | 1.00 | 83.39 | C | |
| ATOM | 602 | O | LEU | A | 273 | 34.313 | −2.435 | 47.214 | 1.00 | 83.53 | O | |
| ATOM | 604 | N | LYS | A | 274 | 35.938 | −3.915 | 47.731 | 1.00 | 84.79 | N | |
| ATOM | 605 | CA | LYS | A | 274 | 35.287 | −4.441 | 48.951 | 1.00 | 85.58 | C | |
| ATOM | 607 | CB | LYS | A | 274 | 36.033 | −5.677 | 49.490 | 1.00 | 85.72 | C | |
| ATOM | 610 | CG | LYS | A | 274 | 36.039 | −6.904 | 48.557 | 1.00 | 85.83 | C | |
| ATOM | 613 | CD | LYS | A | 274 | 36.906 | −8.036 | 49.155 | 1.00 | 85.99 | C | |
| ATOM | 616 | CE | LYS | A | 274 | 37.466 | −9.011 | 48.113 | 1.00 | 86.03 | C | |
| ATOM | 619 | NZ | LYS | A | 274 | 38.640 | −9.761 | 48.665 | 1.00 | 85.15 | N | |
| ATOM | 623 | C | LYS | A | 274 | 35.210 | −3.362 | 50.058 | 1.00 | 86.26 | C | |
| ATOM | 624 | O | LYS | A | 274 | 36.247 | −2.983 | 50.620 | 1.00 | 86.20 | O | |
| ATOM | 626 | N | GLU | A | 275 | 33.981 | −2.893 | 50.322 | 1.00 | 86.92 | N | |
| ATOM | 627 | CA | GLU | A | 275 | 33.599 | −1.857 | 51.336 | 1.00 | 87.61 | C | |
| ATOM | 629 | CB | GLU | A | 275 | 32.345 | −2.327 | 52.135 | 1.00 | 87.78 | C | |
| ATOM | 632 | CG | GLU | A | 275 | 32.345 | −3.772 | 52.662 | 1.00 | 88.17 | C | |
| ATOM | 635 | CD | GLU | A | 275 | 30.958 | −4.427 | 52.683 | 1.00 | 87.94 | C | |
| ATOM | 636 | OE1 | GLU | A | 275 | 30.216 | −4.329 | 51.676 | 1.00 | 87.74 | O | |
| ATOM | 637 | OE2 | GLU | A | 275 | 30.611 | −5.065 | 53.704 | 1.00 | 88.21 | O | |
| ATOM | 638 | C | GLU | A | 275 | 34.635 | −1.203 | 52.299 | 1.00 | 88.06 | C | |
| ATOM | 639 | O | GLU | A | 275 | 34.982 | −0.026 | 52.111 | 1.00 | 88.02 | O | |
| ATOM | 641 | N | ASP | A | 276 | 35.098 | −1.959 | 53.306 | 1.00 | 88.51 | N | |
| ATOM | 642 | CA | ASP | A | 276 | 35.996 | −1.429 | 54.363 | 1.00 | 88.89 | C | |
| ATOM | 644 | CB | ASP | A | 276 | 36.169 | −2.432 | 55.524 | 1.00 | 88.64 | C | |
| ATOM | 647 | CG | ASP | A | 276 | 35.189 | −2.193 | 56.648 | 1.00 | 88.36 | C | |
| ATOM | 648 | OD1 | ASP | A | 276 | 33.987 | −2.103 | 56.347 | 1.00 | 88.75 | O | |
| ATOM | 649 | OD2 | ASP | A | 276 | 35.519 | −2.081 | 57.854 | 1.00 | 86.74 | O | |
| ATOM | 650 | C | ASP | A | 276 | 37.385 | −0.997 | 53.876 | 1.00 | 89.58 | C | |
| ATOM | 651 | O | ASP | A | 276 | 37.790 | 0.147 | 54.140 | 1.00 | 89.49 | O | |
| ATOM | 653 | N | THR | A | 277 | 38.080 | −1.910 | 53.171 | 1.00 | 90.48 | N | |
| ATOM | 654 | CA | THR | A | 277 | 39.526 | −1.776 | 52.795 | 1.00 | 90.67 | C | |
| ATOM | 656 | CB | THR | A | 277 | 40.045 | −3.008 | 51.970 | 1.00 | 90.64 | C | |
| ATOM | 658 | OG1 | THR | A | 277 | 39.129 | −3.335 | 50.916 | 1.00 | 89.62 | O | |
| ATOM | 660 | CG2 | THR | A | 277 | 40.110 | −4.275 | 52.855 | 1.00 | 90.49 | C | |
| ATOM | 664 | C | THR | A | 277 | 39.872 | −0.465 | 52.059 | 1.00 | 91.07 | C | |
| ATOM | 665 | O | THR | A | 277 | 39.463 | −0.271 | 50.911 | 1.00 | 90.51 | O | |
| ATOM | 667 | N | MET | A | 278 | 40.638 | 0.406 | 52.750 | 1.00 | 91.74 | N | |
| ATOM | 668 | CA | MET | A | 278 | 40.877 | 1.815 | 52.356 | 1.00 | 91.84 | C | |
| ATOM | 670 | CB | MET | A | 278 | 41.070 | 2.741 | 53.585 | 1.00 | 91.87 | C | |
| ATOM | 673 | CO | MET | A | 278 | 39.814 | 2.976 | 54.461 | 1.00 | 91.93 | C | |
| ATOM | 676 | SD | MET | A | 278 | 40.170 | 3.936 | 55.999 | 1.00 | 92.15 | S | |
| ATOM | 677 | CE | MET | A | 278 | 38.531 | 4.195 | 56.708 | 1.00 | 91.95 | C | |
| ATOM | 681 | C | MET | A | 278 | 42.038 | 1.973 | 51.340 | 1.00 | 92.19 | C | |
| ATOM | 682 | O | MET | A | 278 | 43.107 | 2.525 | 51.641 | 1.00 | 92.28 | O | |
| ATOM | 684 | N | GLU | A | 279 | 41.791 | 1.443 | 50.137 | 1.00 | 92.25 | N | |
| ATOM | 685 | CA | GLU | A | 279 | 42.265 | 2.042 | 48.896 | 1.00 | 91.93 | C | |
| ATOM | 687 | CB | GLU | A | 279 | 42.763 | 0.956 | 47.919 | 1.00 | 92.14 | C | |
| ATOM | 690 | CG | GLU | A | 279 | 43.976 | 0.161 | 48.419 | 1.00 | 92.21 | C | |
| ATOM | 693 | CD | GLU | A | 279 | 43.608 | −1.147 | 49.106 | 1.00 | 91.94 | C | |
| ATOM | 694 | OE1 | GLU | A | 279 | 43.319 | −1.121 | 50.326 | 1.00 | 91.54 | O | |
| ATOM | 695 | OE2 | GLU | A | 279 | 43.621 | −2.199 | 48.422 | 1.00 | 90.77 | O | |
| ATOM | 696 | C | GLU | A | 279 | 41.104 | 2.865 | 48.296 | 1.00 | 91.66 | C | |
| ATOM | 697 | O | GLU | A | 279 | 41.034 | 3.025 | 47.082 | 1.00 | 91.72 | O | |
| ATOM | 699 | N | VAL | A | 280 | 40.228 | 3.409 | 49.158 | 1.00 | 91.38 | N | |
| ATOM | 700 | CA | VAL | A | 280 | 39.082 | 4.262 | 48.790 | 1.00 | 91.44 | C | |
| ATOM | 702 | CB | VAL | A | 280 | 38.187 | 4.573 | 50.059 | 1.00 | 91.62 | C | |
| ATOM | 704 | CG1 | VAL | A | 280 | 37.117 | 5.649 | 49.795 | 1.00 | 92.06 | C | |
| ATOM | 708 | CG2 | VAL | A | 280 | 37.513 | 3.299 | 50.569 | 1.00 | 92.20 | C | |
| ATOM | 712 | C | VAL | A | 280 | 39.610 | 5.565 | 48.157 | 1.00 | 90.87 | C | |
| ATOM | 713 | O | VAL | A | 280 | 39.104 | 6.025 | 47.127 | 1.00 | 90.63 | O | |
| ATOM | 715 | N | GLU | A | 281 | 40.623 | 6.135 | 48.813 | 1.00 | 90.12 | N | |
| ATOM | 716 | CA | GLU | A | 281 | 41.472 | 7.205 | 48.284 | 1.00 | 89.59 | C | |
| ATOM | 718 | CB | GLU | A | 281 | 42.694 | 7.408 | 49.229 | 1.00 | 89.64 | C | |
| ATOM | 721 | CG | GLU | A | 281 | 43.697 | 8.487 | 48.845 | 1.00 | 89.59 | C | |
| ATOM | 724 | CD | GLU | A | 281 | 43.055 | 9.851 | 48.741 | 1.00 | 89.08 | C | |
| ATOM | 725 | OE1 | GLU | A | 281 | 42.905 | 10.507 | 49.789 | 1.00 | 89.29 | O | |
| ATOM | 726 | OE2 | GLU | A | 281 | 42.691 | 10.258 | 47.615 | 1.00 | 88.22 | O | |
| ATOM | 727 | C | GLU | A | 281 | 41.937 | 6.866 | 46.873 | 1.00 | 88.92 | C | |
| ATOM | 728 | O | GLU | A | 281 | 41.589 | 7.555 | 45.918 | 1.00 | 88.87 | O | |
| ATOM | 730 | N | GLU | A | 282 | 42.658 | 5.756 | 46.765 | 1.00 | 88.39 | N | |
| ATOM | 731 | CA | GLU | A | 282 | 43.383 | 5.368 | 45.542 | 1.00 | 87.76 | C | |
| ATOM | 733 | CB | GLU | A | 282 | 44.440 | 4.297 | 45.922 | 1.00 | 88.40 | C | |
| ATOM | 736 | CG | GLU | A | 282 | 45.480 | 3.945 | 44.861 | 1.00 | 89.10 | C | |
| ATOM | 739 | CD | GLU | A | 282 | 46.223 | 5.161 | 44.323 | 1.00 | 90.47 | C | |
| ATOM | 740 | OE1 | GLU | A | 282 | 46.624 | 6.032 | 45.149 | 1.00 | 90.12 | O | |
| ATOM | 741 | OE2 | GLU | A | 282 | 46.384 | 5.238 | 43.079 | 1.00 | 90.36 | O | |
| ATOM | 742 | C | GLU | A | 282 | 42.514 | 4.887 | 44.344 | 1.00 | 87.08 | C | |
| ATOM | 743 | O | GLU | A | 282 | 42.950 | 4.992 | 43.194 | 1.00 | 86.60 | O | |
| ATOM | 745 | N | PHE | A | 283 | 41.302 | 4.393 | 44.637 | 1.00 | 86.28 | N | |
| ATOM | 746 | CA | PHE | A | 283 | 40.321 | 3.833 | 43.663 | 1.00 | 85.78 | C | |
| ATOM | 748 | CB | PHE | A | 283 | 39.465 | 2.796 | 44.414 | 1.00 | 85.60 | C | |
| ATOM | 751 | CG | PHE | A | 283 | 38.454 | 2.043 | 43.576 | 1.00 | 86.05 | C | |
| ATOM | 752 | CD1 | PHE | A | 283 | 37.164 | 2.571 | 43.359 | 1.00 | 85.61 | C | |
| ATOM | 754 | CE1 | PHE | A | 283 | 36.204 | 1.846 | 42.620 | 1.00 | 85.32 | C | |
| ATOM | 756 | CZ | PHE | A | 283 | 36.520 | 0.571 | 42.123 | 1.00 | 85.38 | C | |
| ATOM | 758 | CE2 | PHE | A | 283 | 37.792 | 0.017 | 42.364 | 1.00 | 85.62 | C | |
| ATOM | 760 | CD2 | PHE | A | 283 | 38.747 | 0.746 | 43.100 | 1.00 | 85.65 | C | |
| ATOM | 762 | C | PHE | A | 283 | 39.415 | 4.906 | 43.026 | 1.00 | 85.17 | C | |
| ATOM | 763 | O | PHE | A | 283 | 39.115 | 4.836 | 41.837 | 1.00 | 85.20 | O | |
| ATOM | 765 | N | LEU | A | 284 | 38.935 | 5.841 | 43.848 | 1.00 | 84.54 | N | |
| ATOM | 766 | CA | LEU | A | 284 | 38.289 | 7.082 | 43.383 | 1.00 | 84.07 | C | |
| ATOM | 768 | CB | LEU | A | 284 | 37.799 | 7.976 | 44.553 | 1.00 | 84.23 | C | |
| ATOM | 771 | CG | LEU | A | 284 | 36.345 | 7.834 | 45.041 | 1.00 | 84.42 | C | |
| ATOM | 773 | CD1 | LEU | A | 284 | 36.217 | 6.760 | 46.133 | 1.00 | 84.04 | C | |
| ATOM | 777 | CD2 | LEU | A | 284 | 35.792 | 9.191 | 45.545 | 1.00 | 84.18 | C | |
| ATOM | 781 | C | LEU | A | 284 | 39.204 | 7.937 | 42.510 | 1.00 | 83.56 | C | |
| ATOM | 782 | O | LEU | A | 284 | 38.707 | 8.614 | 41.602 | 1.00 | 83.85 | O | |
| ATOM | 784 | N | LYS | A | 285 | 40.506 | 7.962 | 42.826 | 1.00 | 82.50 | N | |
| ATOM | 785 | CA | LYS | A | 285 | 41.501 | 8.664 | 41.988 | 1.00 | 81.94 | C | |
| ATOM | 787 | CB | LYS | A | 285 | 42.900 | 8.725 | 42.637 | 1.00 | 81.87 | C | |
| ATOM | 790 | CG | LYS | A | 285 | 43.101 | 9.916 | 43.589 | 1.00 | 83.18 | C | |
| ATOM | 793 | CD | LYS | A | 285 | 43.577 | 11.243 | 42.886 | 1.00 | 84.29 | C | |
| ATOM | 796 | CE | LYS | A | 285 | 42.473 | 12.020 | 42.103 | 1.00 | 84.41 | C | |
| ATOM | 799 | NZ | LYS | A | 285 | 42.376 | 11.636 | 40.647 | 1.00 | 83.92 | N | |
| ATOM | 803 | C | LYS | A | 285 | 41.590 | 8.055 | 40.585 | 1.00 | 80.49 | C | |
| ATOM | 804 | O | LYS | A | 285 | 41.631 | 8.799 | 39.597 | 1.00 | 80.40 | O | |
| ATOM | 806 | N | GLU | A | 286 | 41.590 | 6.717 | 40.509 | 1.00 | 78.57 | N | |
| ATOM | 807 | CA | GLU | A | 286 | 41.571 | 6.025 | 39.229 | 1.00 | 76.92 | C | |
| ATOM | 809 | CB | GLU | A | 286 | 41.609 | 4.509 | 39.390 | 1.00 | 76.80 | C | |
| ATOM | 812 | CG | GLU | A | 286 | 42.068 | 3.853 | 38.111 | 1.00 | 76.55 | C | |
| ATOM | 815 | CD | GLU | A | 286 | 41.897 | 2.357 | 38.076 | 1.00 | 77.14 | C | |
| ATOM | 816 | OE1 | GLU | A | 286 | 41.944 | 1.694 | 39.145 | 1.00 | 74.02 | O | |
| ATOM | 817 | OE2 | GLU | A | 286 | 41.741 | 1.852 | 36.930 | 1.00 | 79.62 | O | |
| ATOM | 818 | C | GLU | A | 286 | 40.342 | 6.431 | 38.427 | 1.00 | 75.69 | C | |
| ATOM | 819 | O | GLU | A | 286 | 40.471 | 6.886 | 37.293 | 1.00 | 75.74 | O | |
| ATOM | 821 | N | ALA | A | 287 | 39.170 | 6.288 | 39.034 | 1.00 | 74.28 | N | |
| ATOM | 822 | CA | ALA | A | 287 | 37.907 | 6.733 | 38.438 | 1.00 | 73.38 | C | |
| ATOM | 824 | CB | ALA | A | 287 | 36.790 | 6.571 | 39.422 | 1.00 | 73.37 | C | |
| ATOM | 828 | C | ALA | A | 287 | 37.952 | 8.184 | 37.934 | 1.00 | 72.87 | C | |
| ATOM | 829 | O | ALA | A | 287 | 37.603 | 8.441 | 36.796 | 1.00 | 72.37 | O | |
| ATOM | 831 | N | ALA | A | 288 | 38.419 | 9.104 | 38.775 | 1.00 | 72.65 | N | |
| ATOM | 832 | CA | ALA | A | 288 | 38.531 | 10.538 | 38.428 | 1.00 | 72.79 | C | |
| ATOM | 834 | CB | ALA | A | 288 | 39.084 | 11.327 | 39.614 | 1.00 | 72.97 | C | |
| ATOM | 838 | C | ALA | A | 288 | 39.409 | 10.792 | 37.203 | 1.00 | 72.62 | C | |
| ATOM | 839 | O | ALA | A | 288 | 39.117 | 11.664 | 36.387 | 1.00 | 73.00 | O | |
| ATOM | 841 | N | VAL | A | 289 | 40.493 | 10.031 | 37.102 | 1.00 | 72.27 | N | |
| ATOM | 842 | CA | VAL | A | 289 | 41.428 | 10.123 | 35.987 | 1.00 | 71.86 | C | |
| ATOM | 844 | CB | VAL | A | 289 | 42.725 | 9.265 | 36.267 | 1.00 | 71.83 | C | |
| ATOM | 846 | CG1 | VAL | A | 289 | 43.555 | 9.016 | 35.004 | 1.00 | 72.53 | C | |
| ATOM | 850 | CG2 | VAL | A | 289 | 43.573 | 9.934 | 37.310 | 1.00 | 71.78 | C | |
| ATOM | 854 | C | VAL | A | 289 | 40.698 | 9.693 | 34.707 | 1.00 | 71.68 | C | |
| ATOM | 855 | O | VAL | A | 289 | 40.582 | 10.489 | 33.769 | 1.00 | 71.31 | O | |
| ATOM | 857 | N | MET | A | 290 | 40.194 | 8.451 | 34.716 | 1.00 | 71.41 | N | |
| ATOM | 858 | CA | MET | A | 290 | 39.414 | 7.866 | 33.607 | 1.00 | 71.13 | C | |
| ATOM | 860 | CB | MET | A | 290 | 38.777 | 6.552 | 34.027 | 1.00 | 70.54 | C | |
| ATOM | 863 | CG | MET | A | 290 | 39.772 | 5.449 | 34.104 | 1.00 | 70.96 | C | |
| ATOM | 866 | SD | MET | A | 290 | 39.063 | 3.871 | 33.716 | 1.00 | 71.69 | S | |
| ATOM | 867 | CE | MET | A | 290 | 39.144 | 3.123 | 35.325 | 1.00 | 72.91 | C | |
| ATOM | 871 | C | MET | A | 290 | 38.329 | 8.765 | 33.020 | 1.00 | 71.20 | C | |
| ATOM | 872 | O | MET | A | 290 | 38.183 | 8.778 | 31.804 | 1.00 | 71.87 | O | |
| ATOM | 874 | N | LYS | A | 291 | 37.591 | 9.492 | 33.878 | 1.00 | 70.87 | N | |
| ATOM | 875 | CA | LYS | A | 291 | 36.621 | 10.520 | 33.455 | 1.00 | 70.54 | C | |
| ATOM | 877 | CB | LYS | A | 291 | 36.052 | 11.257 | 34.662 | 1.00 | 70.45 | C | |
| ATOM | 880 | CG | LYS | A | 291 | 35.052 | 10.482 | 35.497 | 1.00 | 70.39 | C | |
| ATOM | 883 | CD | LYS | A | 291 | 34.522 | 11.383 | 36.630 | 1.00 | 70.23 | C | |
| ATOM | 886 | CE | LYS | A | 291 | 33.411 | 10.743 | 37.460 | 1.00 | 70.38 | C | |
| ATOM | 889 | NZ | LYS | A | 291 | 33.857 | 10.291 | 38.810 | 1.00 | 70.64 | N | |
| ATOM | 893 | C | LYS | A | 291 | 37.189 | 11.573 | 32.493 | 1.00 | 70.72 | C | |
| ATOM | 894 | O | LYS | A | 291 | 36.508 | 11.949 | 31.557 | 1.00 | 71.37 | O | |
| ATOM | 896 | N | GLU | A | 292 | 38.422 | 12.027 | 32.740 | 1.00 | 70.66 | N | |
| ATOM | 897 | CA | GLU | A | 292 | 39.121 | 13.061 | 31.929 | 1.00 | 70.19 | C | |
| ATOM | 899 | CB | GLU | A | 292 | 40.274 | 13.716 | 32.756 | 1.00 | 71.34 | C | |
| ATOM | 902 | CG | GLU | A | 292 | 39.844 | 14.600 | 33.924 | 1.00 | 72.52 | C | |
| ATOM | 905 | CD | GLU | A | 292 | 38.824 | 15.634 | 33.489 | 1.00 | 76.37 | C | |
| ATOM | 906 | OE1 | GLU | A | 292 | 39.064 | 16.293 | 32.443 | 1.00 | 78.86 | O | |
| ATOM | 907 | OE2 | GLU | A | 292 | 37.759 | 15.753 | 34.150 | 1.00 | 79.36 | O | |
| ATOM | 908 | C | GLU | A | 292 | 39.726 | 12.617 | 30.602 | 1.00 | 69.45 | C | |
| ATOM | 909 | O | GLU | A | 292 | 40.089 | 13.483 | 29.798 | 1.00 | 68.68 | O | |
| ATOM | 911 | N | ILE | A | 293 | 39.847 | 11.297 | 30.391 | 1.00 | 68.93 | N | |
| ATOM | 912 | CA | ILE | A | 293 | 40.522 | 10.703 | 29.207 | 1.00 | 68.37 | C | |
| ATOM | 914 | CB | ILE | A | 293 | 41.723 | 9.744 | 29.585 | 1.00 | 68.22 | C | |
| ATOM | 916 | CG1 | ILE | A | 293 | 41.284 | 8.561 | 30.478 | 1.00 | 68.00 | C | |
| ATOM | 919 | CD1 | ILE | A | 293 | 42.408 | 7.534 | 30.814 | 1.00 | 68.00 | C | |
| ATOM | 923 | CG2 | ILE | A | 293 | 42.904 | 10.551 | 30.182 | 1.00 | 67.58 | C | |
| ATOM | 927 | C | ILE | A | 293 | 39.533 | 9.946 | 28.344 | 1.00 | 67.75 | C | |
| ATOM | 928 | O | ILE | A | 293 | 38.601 | 9.331 | 28.868 | 1.00 | 67.66 | O | |
| ATOM | 930 | N | LYS | A | 294 | 39.764 | 9.984 | 27.027 | 1.00 | 67.16 | N | |
| ATOM | 931 | CA | LYS | A | 294 | 38.852 | 9.416 | 26.018 | 1.00 | 66.81 | C | |
| ATOM | 933 | CB | LYS | A | 294 | 37.611 | 10.318 | 25.852 | 1.00 | 66.74 | C | |
| ATOM | 936 | CG | LYS | A | 294 | 36.504 | 9.737 | 25.024 | 1.00 | 66.80 | C | |
| ATOM | 939 | CD | LYS | A | 294 | 35.379 | 10.722 | 24.811 | 1.00 | 67.87 | C | |
| ATOM | 942 | CE | LYS | A | 294 | 34.162 | 10.028 | 24.184 | 1.00 | 69.17 | C | |
| ATOM | 945 | NZ | LYS | A | 294 | 33.543 | 9.072 | 25.184 | 1.00 | 70.26 | N | |
| ATOM | 949 | C | LYS | A | 294 | 39.609 | 9.331 | 24.693 | 1.00 | 66.21 | C | |
| ATOM | 950 | O | LYS | A | 294 | 40.126 | 10.335 | 24.220 | 1.00 | 66.76 | O | |
| ATOM | 952 | N | HIS | A | 295 | 39.684 | 8.140 | 24.111 | 1.00 | 65.16 | N | |
| ATOM | 953 | CA | HIS | A | 295 | 40.510 | 7.880 | 22.920 | 1.00 | 64.11 | C | |
| ATOM | 955 | CB | HIS | A | 295 | 41.991 | 8.048 | 23.269 | 1.00 | 63.82 | C | |
| ATOM | 958 | CG | HIS | A | 295 | 42.905 | 7.962 | 22.100 | 1.00 | 63.30 | C | |
| ATOM | 959 | ND1 | HIS | A | 295 | 43.278 | 6.768 | 21.535 | 1.00 | 62.33 | N | |
| ATOM | 961 | CE1 | HIS | A | 295 | 44.095 | 6.996 | 20.528 | 1.00 | 62.75 | C | |
| ATOM | 963 | NE2 | HIS | A | 295 | 44.282 | 8.300 | 20.434 | 1.00 | 62.37 | N | |
| ATOM | 965 | CD2 | HIS | A | 295 | 43.547 | 8.925 | 21.404 | 1.00 | 62.78 | C | |
| ATOM | 967 | C | HIS | A | 295 | 40.218 | 6.427 | 22.498 | 1.00 | 64.01 | C | |
| ATOM | 968 | O | HIS | A | 295 | 40.043 | 5.565 | 23.381 | 1.00 | 64.08 | O | |
| ATOM | 970 | N | PRO | A | 296 | 40.154 | 6.111 | 21.201 | 1.00 | 63.65 | N | |
| ATOM | 971 | CA | PRO | A | 296 | 39.740 | 4.734 | 20.807 | 1.00 | 63.45 | C | |
| ATOM | 973 | CB | PRO | A | 296 | 39.514 | 4.862 | 19.300 | 1.00 | 63.16 | C | |
| ATOM | 976 | CG | PRO | A | 296 | 40.433 | 5.970 | 18.907 | 1.00 | 63.20 | C | |
| ATOM | 979 | CD | PRO | A | 296 | 40.392 | 6.960 | 20.015 | 1.00 | 63.29 | C | |
| ATOM | 982 | C | PRO | A | 296 | 40.720 | 3.583 | 21.158 | 1.00 | 63.15 | C | |
| ATOM | 983 | O | PRO | A | 296 | 40.373 | 2.415 | 20.965 | 1.00 | 62.79 | O | |
| ATOM | 984 | N | ASN | A | 297 | 41.911 | 3.943 | 21.665 | 1.00 | 63.33 | N | |
| ATOM | 985 | CA | ASN | A | 297 | 42.987 | 3.049 | 22.153 | 1.00 | 62.79 | C | |
| ATOM | 987 | CB | ASN | A | 297 | 44.248 | 3.329 | 21.344 | 1.00 | 62.18 | C | |
| ATOM | 990 | CG | ASN | A | 297 | 44.099 | 2.982 | 19.886 | 1.00 | 61.52 | C | |
| ATOM | 991 | OD1 | ASN | A | 297 | 44.534 | 3.737 | 19.023 | 1.00 | 61.60 | O | |
| ATOM | 992 | ND2 | ASN | A | 297 | 43.518 | 1.830 | 19.601 | 1.00 | 59.64 | N | |
| ATOM | 995 | C | ASN | A | 297 | 43.273 | 3.197 | 23.674 | 1.00 | 62.80 | C | |
| ATOM | 996 | O | ASN | A | 297 | 44.376 | 2.858 | 24.149 | 1.00 | 62.69 | O | |
| ATOM | 998 | N | LEU | A | 298 | 42.291 | 3.720 | 24.418 | 1.00 | 62.50 | N | |
| ATOM | 999 | CA | LEU | A | 298 | 42.278 | 3.691 | 25.869 | 1.00 | 63.06 | C | |
| ATOM | 1001 | CB | LEU | A | 298 | 42.342 | 5.106 | 26.432 | 1.00 | 63.37 | C | |
| ATOM | 1004 | CG | LEU | A | 298 | 43.577 | 5.945 | 26.111 | 1.00 | 64.23 | C | |
| ATOM | 1006 | CD1 | LEU | A | 298 | 43.403 | 7.348 | 26.624 | 1.00 | 64.50 | C | |
| ATOM | 1010 | CD2 | LEU | A | 298 | 44.805 | 5.338 | 26.721 | 1.00 | 65.89 | C | |
| ATOM | 1014 | C | LEU | A | 298 | 40.982 | 3.039 | 26.333 | 1.00 | 63.13 | C | |
| ATOM | 1015 | O | LEU | A | 298 | 39.922 | 3.464 | 25.905 | 1.00 | 64.25 | O | |
| ATOM | 1017 | N | VAL | A | 299 | 41.047 | 2.066 | 27.238 | 1.00 | 62.67 | N | |
| ATOM | 1018 | CA | VAL | A | 299 | 39.845 | 1.394 | 27.732 | 1.00 | 62.56 | C | |
| ATOM | 1020 | CB | VAL | A | 299 | 40.185 | 0.406 | 28.834 | 1.00 | 62.14 | C | |
| ATOM | 1022 | CG1 | VAL | A | 299 | 38.919 | −0.124 | 29.535 | 1.00 | 62.99 | C | |
| ATOM | 1026 | CG2 | VAL | A | 299 | 40.955 | −0.729 | 28.260 | 1.00 | 62.66 | C | |
| ATOM | 1030 | C | VAL | A | 299 | 38.897 | 2.445 | 28.291 | 1.00 | 62.79 | C | |
| ATOM | 1031 | O | VAL | A | 299 | 39.328 | 3.274 | 29.047 | 1.00 | 63.51 | O | |
| ATOM | 1033 | N | GLN | A | 300 | 37.628 | 2.400 | 27.913 | 1.00 | 63.32 | N | |
| ATOM | 1034 | CA | GLN | A | 300 | 36.685 | 3.500 | 28.127 | 1.00 | 63.63 | C | |
| ATOM | 1036 | CB | GLN | A | 300 | 35.756 | 3.675 | 26.917 | 1.00 | 63.32 | C | |
| ATOM | 1039 | CG | GLN | A | 300 | 34.685 | 4.816 | 27.034 | 1.00 | 63.33 | C | |
| ATOM | 1042 | CD | GLN | A | 300 | 35.263 | 6.198 | 27.379 | 1.00 | 61.22 | C | |
| ATOM | 1043 | OE1 | GLN | A | 300 | 35.041 | 6.720 | 28.456 | 1.00 | 59.23 | O | |
| ATOM | 1044 | NE2 | GLN | A | 300 | 36.002 | 6.769 | 26.461 | 1.00 | 61.49 | N | |
| ATOM | 1047 | C | GLN | A | 300 | 35.854 | 3.259 | 29.381 | 1.00 | 64.19 | C | |
| ATOM | 1048 | O | GLN | A | 300 | 35.225 | 2.217 | 29.514 | 1.00 | 63.96 | O | |
| ATOM | 1050 | N | LEU | A | 301 | 35.866 | 4.245 | 30.283 | 1.00 | 65.01 | N | |
| ATOM | 1051 | CA | LEU | A | 301 | 34.946 | 4.310 | 31.400 | 1.00 | 65.66 | C | |
| ATOM | 1053 | CB | LEU | A | 301 | 35.311 | 5.484 | 32.307 | 1.00 | 65.87 | C | |
| ATOM | 1056 | CG | LEU | A | 301 | 34.454 | 5.771 | 33.555 | 1.00 | 65.55 | C | |
| ATOM | 1058 | CD1 | LEU | A | 301 | 34.585 | 4.634 | 34.534 | 1.00 | 64.53 | C | |
| ATOM | 1062 | CD2 | LEU | A | 301 | 34.850 | 7.107 | 34.212 | 1.00 | 65.68 | C | |
| ATOM | 1066 | C | LEU | A | 301 | 33.522 | 4.512 | 30.865 | 1.00 | 66.65 | C | |
| ATOM | 1067 | O | LEU | A | 301 | 33.227 | 5.528 | 30.210 | 1.00 | 66.88 | O | |
| ATOM | 1069 | N | LEU | A | 302 | 32.648 | 3.545 | 31.142 | 1.00 | 67.04 | N | |
| ATOM | 1070 | CA | LEU | A | 302 | 31.225 | 3.652 | 30.801 | 1.00 | 66.76 | C | |
| ATOM | 1072 | CB | LEU | A | 302 | 30.670 | 2.307 | 30.378 | 1.00 | 66.20 | C | |
| ATOM | 1075 | CG | LEU | A | 302 | 31.392 | 1.728 | 29.156 | 1.00 | 65.50 | C | |
| ATOM | 1077 | CD1 | LEU | A | 302 | 30.845 | 0.348 | 28.924 | 1.00 | 66.48 | C | |
| ATOM | 1081 | CD2 | LEU | A | 302 | 31.259 | 2.583 | 27.887 | 1.00 | 64.32 | C | |
| ATOM | 1085 | C | LEU | A | 302 | 30.433 | 4.220 | 31.959 | 1.00 | 67.32 | C | |
| ATOM | 1086 | O | LEU | A | 302 | 29.543 | 5.030 | 31.731 | 1.00 | 67.57 | O | |
| ATOM | 1088 | N | GLY | A | 303 | 30.753 | 3.822 | 33.193 | 1.00 | 68.14 | N | |
| ATOM | 1089 | CA | GLY | A | 303 | 29.989 | 4.287 | 34.367 | 1.00 | 68.66 | C | |
| ATOM | 1092 | C | GLY | A | 303 | 30.540 | 3.874 | 35.699 | 1.00 | 69.03 | C | |
| ATOM | 1093 | O | GLY | A | 303 | 31.467 | 3.067 | 35.748 | 1.00 | 68.17 | O | |
| ATOM | 1095 | N | VAL | A | 304 | 29.951 | 4.421 | 36.773 | 1.00 | 70.26 | N | |
| ATOM | 1096 | CA | VAL | A | 304 | 30.425 | 4.182 | 38.163 | 1.00 | 71.13 | C | |
| ATOM | 1098 | CB | VAL | A | 304 | 31.456 | 5.263 | 38.660 | 1.00 | 71.03 | C | |
| ATOM | 1100 | CG1 | VAL | A | 304 | 32.751 | 5.176 | 37.892 | 1.00 | 70.35 | C | |
| ATOM | 1104 | CG2 | VAL | A | 304 | 30.868 | 6.707 | 38.606 | 1.00 | 71.13 | C | |
| ATOM | 1108 | C | VAL | A | 304 | 29.327 | 4.097 | 39.212 | 1.00 | 72.12 | C | |
| ATOM | 1109 | O | VAL | A | 304 | 28.233 | 4.594 | 39.006 | 1.00 | 72.20 | O | |
| ATOM | 1111 | N | CYS | A | 305 | 29.657 | 3.453 | 40.333 | 1.00 | 73.76 | N | |
| ATOM | 1112 | CA | CYS | A | 305 | 28.875 | 3.495 | 41.584 | 1.00 | 75.38 | C | |
| ATOM | 1114 | CB | CYS | A | 305 | 28.204 | 2.152 | 41.846 | 1.00 | 75.15 | C | |
| ATOM | 1117 | SG | CYS | A | 305 | 27.535 | 1.430 | 40.364 | 1.00 | 74.26 | S | |
| ATOM | 1119 | C | CYS | A | 305 | 29.812 | 3.879 | 42.751 | 1.00 | 76.29 | C | |
| ATOM | 1120 | O | CYS | A | 305 | 30.569 | 3.036 | 43.257 | 1.00 | 76.41 | O | |
| ATOM | 1122 | N | THR | A | 306 | 29.793 | 5.166 | 43.111 | 1.00 | 77.27 | N | |
| ATOM | 1123 | CA | THR | A | 306 | 30.635 | 5.749 | 44.166 | 1.00 | 78.12 | C | |
| ATOM | 1125 | CB | THR | A | 306 | 31.701 | 6.687 | 43.537 | 1.00 | 78.19 | C | |
| ATOM | 1127 | OG1 | THR | A | 306 | 31.067 | 7.781 | 42.862 | 1.00 | 78.61 | O | |
| ATOM | 1129 | CG2 | THR | A | 306 | 32.499 | 5.995 | 42.440 | 1.00 | 78.02 | C | |
| ATOM | 1133 | C | THR | A | 306 | 29.858 | 6.517 | 45.260 | 1.00 | 79.18 | C | |
| ATOM | 1134 | O | THR | A | 306 | 30.475 | 7.091 | 46.169 | 1.00 | 79.58 | O | |
| ATOM | 1136 | N | ARG | A | 307 | 28.523 | 6.523 | 45.186 | 1.00 | 79.97 | N | |
| ATOM | 1137 | CA | ARG | A | 307 | 27.679 | 7.102 | 46.232 | 1.00 | 80.18 | C | |
| ATOM | 1139 | CB | ARG | A | 307 | 26.207 | 7.111 | 45.821 | 1.00 | 80.34 | C | |
| ATOM | 1142 | CG | ARG | A | 307 | 25.892 | 7.891 | 44.601 | 1.00 | 80.53 | C | |
| ATOM | 1145 | CD | ARG | A | 307 | 25.630 | 9.359 | 44.791 | 1.00 | 81.08 | C | |
| ATOM | 1148 | NE | ARG | A | 307 | 25.410 | 9.968 | 43.468 | 1.00 | 81.40 | N | |
| ATOM | 1150 | CZ | ARG | A | 307 | 24.657 | 11.040 | 43.188 | 1.00 | 81.22 | C | |
| ATOM | 1151 | NH1 | ARG | A | 307 | 23.980 | 11.714 | 44.133 | 1.00 | 80.50 | N | |
| ATOM | 1154 | NH2 | ARG | A | 307 | 24.577 | 11.439 | 41.912 | 1.00 | 81.42 | N | |
| ATOM | 1157 | C | ARG | A | 307 | 27.811 | 6.237 | 47.477 | 1.00 | 81.10 | C | |
| ATOM | 1158 | O | ARG | A | 307 | 28.081 | 6.752 | 48.565 | 1.00 | 81.00 | O | |
| ATOM | 1160 | N | GLU | A | 308 | 27.655 | 4.921 | 47.268 | 1.00 | 81.86 | N | |
| ATOM | 1161 | CA | GLU | A | 308 | 27.400 | 3.921 | 48.319 | 1.00 | 82.42 | C | |
| ATOM | 1163 | CB | GLU | A | 308 | 25.889 | 3.538 | 48.324 | 1.00 | 82.41 | C | |
| ATOM | 1166 | CG | GLU | A | 308 | 24.962 | 4.452 | 47.501 | 1.00 | 82.71 | C | |
| ATOM | 1169 | CD | GLU | A | 308 | 23.480 | 4.117 | 47.591 | 1.00 | 83.00 | C | |
| ATOM | 1170 | OE1 | GLU | A | 308 | 22.850 | 4.336 | 48.665 | 1.00 | 82.97 | O | |
| ATOM | 1171 | OE2 | GLU | A | 308 | 22.935 | 3.661 | 46.554 | 1.00 | 83.49 | O | |
| ATOM | 1172 | C | GLU | A | 308 | 28.238 | 2.668 | 48.003 | 1.00 | 82.54 | C | |
| ATOM | 1173 | O | GLU | A | 308 | 28.139 | 2.185 | 46.882 | 1.00 | 83.35 | O | |
| ATOM | 1175 | N | PRO | A | 309 | 29.070 | 2.146 | 48.916 | 1.00 | 82.70 | N | |
| ATOM | 1176 | CA | PRO | A | 309 | 29.683 | 0.792 | 48.731 | 1.00 | 82.65 | C | |
| ATOM | 1178 | CB | PRO | A | 309 | 30.504 | 0.595 | 50.015 | 1.00 | 82.72 | C | |
| ATOM | 1181 | CG | PRO | A | 309 | 30.808 | 1.986 | 50.483 | 1.00 | 82.78 | C | |
| ATOM | 1184 | CD | PRO | A | 309 | 29.594 | 2.807 | 50.125 | 1.00 | 82.73 | C | |
| ATOM | 1187 | C | PRO | A | 309 | 28.692 | −0.405 | 48.511 | 1.00 | 82.39 | C | |
| ATOM | 1188 | O | PRO | A | 309 | 27.467 | −0.191 | 48.577 | 1.00 | 82.22 | O | |
| ATOM | 1189 | N | PRO | A | 310 | 29.193 | −1.637 | 48.268 | 1.00 | 82.03 | N | |
| ATOM | 1190 | CA | PRO | A | 310 | 30.388 | −1.934 | 47.408 | 1.00 | 81.56 | C | |
| ATOM | 1192 | CB | PRO | A | 310 | 30.121 | −3.390 | 46.937 | 1.00 | 81.53 | C | |
| ATOM | 1195 | CG | PRO | A | 310 | 28.660 | −3.681 | 47.332 | 1.00 | 81.51 | C | |
| ATOM | 1198 | CD | PRO | A | 310 | 28.452 | −2.885 | 48.579 | 1.00 | 81.90 | C | |
| ATOM | 1201 | C | PRO | A | 310 | 30.600 | −1.034 | 46.184 | 1.00 | 81.28 | C | |
| ATOM | 1202 | O | PRO | A | 310 | 29.702 | −0.946 | 45.371 | 1.00 | 81.70 | O | |
| ATOM | 1203 | N | PHE | A | 311 | 31.753 | −0.368 | 46.071 | 1.00 | 80.84 | N | |
| ATOM | 1204 | CA | PHE | A | 311 | 32.013 | 0.562 | 44.941 | 1.00 | 80.50 | C | |
| ATOM | 1206 | CB | PHE | A | 311 | 33.229 | 1.468 | 45.215 | 1.00 | 81.26 | C | |
| ATOM | 1209 | CG | PHE | A | 311 | 33.056 | 2.466 | 46.341 | 1.00 | 81.65 | C | |
| ATOM | 1210 | CD1 | PHE | A | 311 | 31.836 | 3.143 | 46.568 | 1.00 | 83.07 | C | |
| ATOM | 1212 | CE1 | PHE | A | 311 | 31.715 | 4.100 | 47.602 | 1.00 | 83.08 | C | |
| ATOM | 1214 | CZ | PHE | A | 311 | 32.832 | 4.407 | 48.406 | 1.00 | 83.12 | C | |
| ATOM | 1216 | CE2 | PHE | A | 311 | 34.060 | 3.753 | 48.175 | 1.00 | 82.98 | C | |
| ATOM | 1218 | CD2 | PHE | A | 311 | 34.162 | 2.795 | 47.138 | 1.00 | 82.23 | C | |
| ATOM | 1220 | C | PHE | A | 311 | 32.287 | −0.165 | 43.616 | 1.00 | 80.01 | C | |
| ATOM | 1221 | O | PHE | A | 311 | 32.975 | −1.198 | 43.638 | 1.00 | 80.09 | O | |
| ATOM | 1223 | N | TYR | A | 312 | 31.795 | 0.403 | 42.488 | 1.00 | 78.96 | N | |
| ATOM | 1224 | CA | TYR | A | 312 | 31.973 | −0.142 | 41.095 | 1.00 | 78.21 | C | |
| ATOM | 1226 | CB | TYR | A | 312 | 30.616 | −0.589 | 40.507 | 1.00 | 78.08 | C | |
| ATOM | 1229 | CG | TYR | A | 312 | 29.934 | −1.847 | 41.023 | 1.00 | 78.36 | C | |
| ATOM | 1230 | CD1 | TYR | A | 312 | 30.598 | −2.783 | 41.846 | 1.00 | 78.53 | C | |
| ATOM | 1232 | CE1 | TYR | A | 312 | 29.947 | −3.945 | 42.289 | 1.00 | 78.74 | C | |
| ATOM | 1234 | CZ | TYR | A | 312 | 28.638 | −4.204 | 41.898 | 1.00 | 77.96 | C | |
| ATOM | 1235 | OH | TYR | A | 312 | 28.058 | −5.353 | 42.365 | 1.00 | 78.24 | O | |
| ATOM | 1237 | CE2 | TYR | A | 312 | 27.955 | −3.315 | 41.062 | 1.00 | 77.95 | C | |
| ATOM | 1239 | CD2 | TYR | A | 312 | 28.606 | −2.141 | 40.626 | 1.00 | 77.80 | C | |
| ATOM | 1241 | C | TYR | A | 312 | 32.536 | 0.870 | 40.066 | 1.00 | 76.60 | C | |
| ATOM | 1242 | O | TYR | A | 312 | 31.951 | 1.915 | 39.887 | 1.00 | 76.21 | O | |
| ATOM | 1244 | N | ILE | A | 313 | 33.637 | 0.542 | 39.388 | 1.00 | 75.47 | N | |
| ATOM | 1245 | CA | ILE | A | 313 | 33.999 | 1.162 | 38.107 | 1.00 | 75.08 | C | |
| ATOM | 1247 | CB | ILE | A | 313 | 35.494 | 1.490 | 38.011 | 1.00 | 74.66 | C | |
| ATOM | 1249 | CG1 | ILE | A | 313 | 35.833 | 2.686 | 38.885 | 1.00 | 75.43 | C | |
| ATOM | 1252 | CD1 | ILE | A | 313 | 37.380 | 2.834 | 39.177 | 1.00 | 76.05 | C | |
| ATOM | 1256 | CG2 | ILE | A | 313 | 35.898 | 1.837 | 36.568 | 1.00 | 74.63 | C | |
| ATOM | 1260 | C | ILE | A | 313 | 33.605 | 0.182 | 36.993 | 1.00 | 74.76 | C | |
| ATOM | 1261 | O | ILE | A | 313 | 34.070 | −0.952 | 36.989 | 1.00 | 74.17 | O | |
| ATOM | 1263 | N | ILE | A | 314 | 32.794 | 0.663 | 36.046 | 1.00 | 74.35 | N | |
| ATOM | 1264 | CA | ILE | A | 314 | 32.342 | −0.090 | 34.888 | 1.00 | 74.17 | C | |
| ATOM | 1266 | CB | ILE | A | 314 | 30.817 | 0.072 | 34.688 | 1.00 | 74.31 | C | |
| ATOM | 1268 | CG1 | ILE | A | 314 | 30.070 | −0.402 | 35.951 | 1.00 | 75.01 | C | |
| ATOM | 1271 | CD1 | ILE | A | 314 | 29.828 | 0.654 | 37.004 | 1.00 | 75.58 | C | |
| ATOM | 1275 | CG2 | ILE | A | 314 | 30.341 | −0.698 | 33.433 | 1.00 | 73.68 | C | |
| ATOM | 1279 | C | ILE | A | 314 | 33.085 | 0.455 | 33.678 | 1.00 | 73.98 | C | |
| ATOM | 1280 | O | ILE | A | 314 | 33.120 | 1.672 | 33.468 | 1.00 | 74.80 | O | |
| ATOM | 1282 | N | THR | A | 315 | 33.682 | −0.437 | 32.894 | 1.00 | 73.15 | N | |
| ATOM | 1283 | CA | THR | A | 315 | 34.313 | −0.075 | 31.627 | 1.00 | 72.37 | C | |
| ATOM | 1285 | CB | THR | A | 315 | 35.875 | −0.050 | 31.729 | 1.00 | 72.39 | C | |
| ATOM | 1287 | OG1 | THR | A | 315 | 36.382 | −1.367 | 31.919 | 1.00 | 71.53 | O | |
| ATOM | 1289 | CG2 | THR | A | 315 | 36.380 | 0.709 | 32.965 | 1.00 | 73.01 | C | |
| ATOM | 1293 | C | THR | A | 315 | 33.888 | −1.046 | 30.537 | 1.00 | 71.97 | C | |
| ATOM | 1294 | O | THR | A | 315 | 33.270 | −2.092 | 30.788 | 1.00 | 71.30 | O | |
| ATOM | 1296 | N | GLU | A | 316 | 34.257 | −0.674 | 29.314 | 1.00 | 71.64 | N | |
| ATOM | 1297 | CA | GLU | A | 316 | 34.007 | −1.484 | 28.146 | 1.00 | 71.30 | C | |
| ATOM | 1299 | CB | GLU | A | 316 | 34.409 | −0.758 | 26.863 | 1.00 | 71.42 | C | |
| ATOM | 1302 | CG | GLU | A | 316 | 35.900 | −0.586 | 26.636 | 1.00 | 72.45 | C | |
| ATOM | 1305 | CD | GLU | A | 316 | 36.220 | 0.227 | 25.389 | 1.00 | 73.75 | C | |
| ATOM | 1306 | OE1 | GLU | A | 316 | 35.465 | 0.194 | 24.394 | 1.00 | 77.94 | O | |
| ATOM | 1307 | OE2 | GLU | A | 316 | 37.266 | 0.900 | 25.377 | 1.00 | 78.81 | O | |
| ATOM | 1308 | C | GLU | A | 316 | 34.785 | −2.751 | 28.297 | 1.00 | 69.81 | C | |
| ATOM | 1309 | O | GLU | A | 316 | 35.849 | −2.728 | 28.870 | 1.00 | 69.06 | O | |
| ATOM | 1311 | N | PHE | A | 317 | 34.220 | −3.835 | 27.785 | 1.00 | 69.21 | N | |
| ATOM | 1312 | CA | PHE | A | 317 | 34.814 | −5.163 | 27.839 | 1.00 | 68.70 | C | |
| ATOM | 1314 | CB | PHE | A | 317 | 33.709 | −6.190 | 27.990 | 1.00 | 68.39 | C | |
| ATOM | 1317 | CG | PHE | A | 317 | 34.190 | −7.569 | 28.187 | 1.00 | 67.49 | C | |
| ATOM | 1318 | CD1 | PHE | A | 317 | 34.851 | −7.917 | 29.346 | 1.00 | 68.51 | C | |
| ATOM | 1320 | CE1 | PHE | A | 317 | 35.289 | −9.243 | 29.550 | 1.00 | 69.21 | C | |
| ATOM | 1322 | CZ | PHE | A | 317 | 35.053 | −10.219 | 28.571 | 1.00 | 68.33 | C | |
| ATOM | 1324 | CE2 | PHE | A | 317 | 34.388 | −9.865 | 27.409 | 1.00 | 68.37 | C | |
| ATOM | 1326 | CD2 | PHE | A | 317 | 33.950 | −8.546 | 27.232 | 1.00 | 68.31 | C | |
| ATOM | 1328 | C | PHE | A | 317 | 35.641 | −5.420 | 26.552 | 1.00 | 69.05 | C | |
| ATOM | 1329 | O | PHE | A | 317 | 35.269 | −4.944 | 25.454 | 1.00 | 69.34 | O | |
| ATOM | 1331 | N | MET | A | 318 | 36.751 | −6.163 | 26.725 | 1.00 | 68.64 | N | |
| ATOM | 1332 | CA | MET | A | 318 | 37.743 | −6.510 | 25.687 | 1.00 | 67.80 | C | |
| ATOM | 1334 | CB | MET | A | 318 | 39.069 | −5.798 | 26.000 | 1.00 | 68.06 | C | |
| ATOM | 1337 | CG | MET | A | 318 | 38.953 | −4.289 | 26.156 | 1.00 | 68.55 | C | |
| ATOM | 1340 | SD | MET | A | 318 | 38.679 | −3.484 | 24.554 | 1.00 | 72.31 | S | |
| ATOM | 1341 | CE | MET | A | 318 | 40.286 | −3.408 | 23.859 | 1.00 | 70.76 | C | |
| ATOM | 1345 | C | MET | A | 318 | 37.913 | −8.050 | 25.700 | 1.00 | 66.94 | C | |
| ATOM | 1346 | O | MET | A | 318 | 38.691 | −8.601 | 26.476 | 1.00 | 66.14 | O | |
| ATOM | 1348 | N | THR | A | 319 | 37.139 | −8.718 | 24.848 | 1.00 | 66.49 | N | |
| ATOM | 1349 | CA | THR | A | 319 | 36.995 | −10.201 | 24.780 | 1.00 | 66.27 | C | |
| ATOM | 1351 | CB | THR | A | 319 | 36.371 | −10.589 | 23.427 | 1.00 | 66.14 | C | |
| ATOM | 1353 | OG1 | THR | A | 319 | 35.224 | −9.776 | 23.196 | 1.00 | 65.99 | O | |
| ATOM | 1355 | CG2 | THR | A | 319 | 35.827 | −12.049 | 23.414 | 1.00 | 66.55 | C | |
| ATOM | 1359 | C | THR | A | 319 | 38.239 | −11.055 | 24.938 | 1.00 | 65.58 | C | |
| ATOM | 1360 | O | THR | A | 319 | 38.151 | −12.131 | 25.529 | 1.00 | 65.37 | O | |
| ATOM | 1362 | N | TYR | A | 320 | 39.362 | −10.590 | 24.373 | 1.00 | 65.07 | N | |
| ATOM | 1363 | CA | TYR | A | 320 | 40.620 | −11.359 | 24.314 | 1.00 | 64.71 | C | |
| ATOM | 1365 | CB | TYR | A | 320 | 41.219 | −11.253 | 22.899 | 1.00 | 65.92 | C | |
| ATOM | 1368 | CG | TYR | A | 320 | 40.383 | −12.013 | 21.883 | 1.00 | 66.66 | C | |
| ATOM | 1369 | CD1 | TYR | A | 320 | 40.394 | −13.422 | 21.855 | 1.00 | 68.48 | C | |
| ATOM | 1371 | CE1 | TYR | A | 320 | 39.590 | −14.163 | 20.920 | 1.00 | 68.46 | C | |
| ATOM | 1373 | CZ | TYR | A | 320 | 38.775 | −13.475 | 20.026 | 1.00 | 68.18 | C | |
| ATOM | 1374 | OH | TYR | A | 320 | 38.018 | −14.199 | 19.137 | 1.00 | 67.52 | O | |
| ATOM | 1376 | CE2 | TYR | A | 320 | 38.748 | −12.062 | 20.048 | 1.00 | 67.89 | C | |
| ATOM | 1378 | CD2 | TYR | A | 320 | 39.545 | −11.342 | 20.975 | 1.00 | 67.64 | C | |
| ATOM | 1380 | C | TYR | A | 320 | 41.651 | −11.030 | 25.394 | 1.00 | 63.30 | C | |
| ATOM | 1381 | O | TYR | A | 320 | 42.745 | −11.600 | 25.401 | 1.00 | 63.09 | O | |
| ATOM | 1383 | N | GLY | A | 321 | 41.304 | −10.121 | 26.303 | 1.00 | 61.92 | N | |
| ATOM | 1384 | CA GLY | A | 321 | 42.048 | −9.926 | 27.541 | 1.00 | 60.67 | C | | |
| ATOM | 1387 | C | GLY | A | 321 | 43.422 | −9.371 | 27.281 | 1.00 | 59.66 | C | |
| ATOM | 1388 | O | GLY | A | 321 | 43.613 | −8.684 | 26.262 | 1.00 | 59.76 | O | |
| ATOM | 1390 | N | ASN | A | 322 | 44.380 | −9.719 | 28.154 | 1.00 | 58.09 | N | |
| ATOM | 1391 | CA | ASN | A | 322 | 45.676 | −9.014 | 28.227 | 1.00 | 56.58 | C | |
| ATOM | 1393 | CB | ASN | A | 322 | 46.337 | −9.102 | 29.624 | 1.00 | 56.33 | C | |
| ATOM | 1396 | CG | ASN | A | 322 | 47.110 | −10.393 | 29.846 | 1.00 | 56.31 | C | |
| ATOM | 1397 | OD1 | ASN | A | 322 | 48.324 | −10.402 | 29.781 | 1.00 | 57.93 | O | |
| ATOM | 1398 | ND2 | ASN | A | 322 | 46.405 | −11.483 | 30.087 | 1.00 | 53.78 | N | |
| ATOM | 1401 | C | ASN | A | 322 | 46.639 | −9.404 | 27.111 | 1.00 | 55.23 | C | |
| ATOM | 1402 | O | ASN | A | 322 | 46.817 | −10.572 | 26.818 | 1.00 | 55.23 | O | |
| ATOM | 1404 | N | LEU | A | 323 | 47.256 | −8.396 | 26.510 | 1.00 | 53.84 | N | |
| ATOM | 1405 | CA | LEU | A | 323 | 48.155 | −8.559 | 25.360 | 1.00 | 53.07 | C | |
| ATOM | 1407 | CB | LEU | A | 323 | 48.701 | −7.196 | 24.927 | 1.00 | 52.47 | C | |
| ATOM | 1410 | CG | LEU | A | 323 | 49.698 | −7.102 | 23.785 | 1.00 | 52.78 | C | |
| ATOM | 1412 | CD1 | LEU | A | 323 | 49.101 | −7.640 | 22.509 | 1.00 | 52.22 | C | |
| ATOM | 1416 | CD2 | LEU | A | 323 | 50.106 | −5.644 | 23.610 | 1.00 | 53.61 | C | |
| ATOM | 1420 | C | LEU | A | 323 | 49.324 | −9.507 | 25.592 | 1.00 | 52.29 | C | |
| ATOM | 1421 | O | LEU | A | 323 | 49.734 | −10.125 | 24.658 | 1.00 | 51.98 | O | |
| ATOM | 1423 | N | LEU | A | 324 | 49.858 | −9.599 | 26.811 | 1.00 | 52.01 | N | |
| ATOM | 1424 | CA | LEU | A | 324 | 50.987 | −10.499 | 27.096 | 1.00 | 52.39 | C | |
| ATOM | 1426 | CB | LEU | A | 324 | 51.576 | −10.263 | 28.491 | 1.00 | 51.92 | C | |
| ATOM | 1429 | CG | LEU | A | 324 | 52.753 | −11.160 | 28.912 | 1.00 | 51.66 | C | |
| ATOM | 1431 | CD1 | LEU | A | 324 | 53.991 | −11.039 | 28.001 | 1.00 | 51.19 | C | |
| ATOM | 1435 | CD2 | LEU | A | 324 | 53.110 | −10.832 | 30.311 | 1.00 | 51.89 | C | |
| ATOM | 1439 | C | LEU | A | 324 | 50.602 | −11.988 | 26.951 | 1.00 | 52.94 | C | |
| ATOM | 1440 | O | LEU | A | 324 | 51.338 | −12.768 | 26.336 | 1.00 | 52.42 | O | |
| ATOM | 1442 | N | ASP | A | 325 | 49.468 | −12.366 | 27.533 | 1.00 | 53.59 | N | |
| ATOM | 1443 | CA | ASP | A | 325 | 48.974 | −13.724 | 27.379 | 1.00 | 54.58 | C | |
| ATOM | 1445 | CB | ASP | A | 325 | 47.966 | −14.090 | 28.465 | 1.00 | 54.98 | C | |
| ATOM | 1448 | CG | ASP | A | 325 | 48.583 | −14.065 | 29.864 | 1.00 | 57.90 | C | |
| ATOM | 1449 | OD1 | ASP | A | 325 | 49.792 | −14.422 | 29.992 | 1.00 | 61.26 | O | |
| ATOM | 1450 | OD2 | ASP | A | 325 | 47.942 | −13.695 | 30.888 | 1.00 | 60.00 | O | |
| ATOM | 1451 | C | ASP | A | 325 | 48.400 | −13.933 | 25.998 | 1.00 | 54.49 | C | |
| ATOM | 1452 | O | ASP | A | 325 | 48.471 | −15.028 | 25.497 | 1.00 | 54.99 | O | |
| ATOM | 1454 | N | TYR | A | 326 | 47.860 | −12.890 | 25.376 | 1.00 | 54.48 | N | |
| ATOM | 1455 | CA | TYR | A | 326 | 47.347 | −12.987 | 24.023 | 1.00 | 54.35 | C | |
| ATOM | 1457 | CB | TYR | A | 326 | 46.728 | −11.666 | 23.586 | 1.00 | 55.11 | C | |
| ATOM | 1460 | CG | TYR | A | 326 | 46.174 | −11.719 | 22.197 | 1.00 | 56.05 | C | |
| ATOM | 1461 | CD1 | TYR | A | 326 | 44.961 | −12.349 | 21.961 | 1.00 | 56.75 | C | |
| ATOM | 1463 | CE1 | TYR | A | 326 | 44.428 | −12.434 | 20.686 | 1.00 | 56.63 | C | |
| ATOM | 1465 | CZ | TYR | A | 326 | 45.102 | −11.867 | 19.622 | 1.00 | 56.70 | C | |
| ATOM | 1466 | OH | TYR | A | 326 | 44.522 | −11.992 | 18.381 | 1.00 | 57.16 | O | |
| ATOM | 1468 | CE2 | TYR | A | 326 | 46.329 | −11.208 | 19.821 | 1.00 | 56.39 | C | |
| ATOM | 1470 | CD2 | TYR | A | 326 | 46.868 | −11.159 | 21.108 | 1.00 | 56.27 | C | |
| ATOM | 1472 | C | TYR | A | 326 | 48.468 | −13.388 | 23.081 | 1.00 | 53.77 | C | |
| ATOM | 1473 | O | TYR | A | 326 | 48.392 | −14.424 | 22.449 | 1.00 | 54.36 | O | |
| ATOM | 1475 | N | LEU | A | 327 | 49.514 | −12.577 | 23.041 | 1.00 | 53.12 | N | |
| ATOM | 1476 | CA | LEU | A | 327 | 50.718 | −12.837 | 22.250 | 1.00 | 52.14 | C | |
| ATOM | 1478 | CB | LEU | A | 327 | 51.782 | −11.815 | 22.588 | 1.00 | 51.78 | C | |
| ATOM | 1481 | CG | LEU | A | 327 | 51.578 | −10.396 | 22.107 | 1.00 | 52.13 | C | |
| ATOM | 1483 | CD1 | LEU | A | 327 | 52.612 | −9.475 | 22.831 | 1.00 | 54.47 | C | |
| ATOM | 1487 | CD2 | LEU | A | 327 | 51.722 | −10.316 | 20.642 | 1.00 | 51.88 | C | |
| ATOM | 1491 | C | LEU | A | 327 | 51.350 | −14.192 | 22.486 | 1.00 | 51.44 | C | |
| ATOM | 1492 | O | LEU | A | 327 | 51.778 | −14.824 | 21.537 | 1.00 | 51.45 | 0 | |
| ATOM | 1494 | N | ARG | A | 328 | 51.456 | −14.596 | 23.747 | 1.00 | 50.98 | N | |
| ATOM | 1495 | CA | ARG | A | 328 | 52.064 | −15.898 | 24.100 | 1.00 | 51.10 | C | |
| ATOM | 1497 | CB | ARG | A | 328 | 52.209 | −16.045 | 25.617 | 1.00 | 50.94 | C | |
| ATOM | 1500 | CG | ARG | A | 328 | 53.410 | −15.397 | 26.249 | 1.00 | 50.36 | C | |
| ATOM | 1503 | CD | ARG | A | 328 | 53.289 | −15.321 | 27.771 | 1.00 | 51.71 | C | |
| ATOM | 1506 | NE | ARG | A | 328 | 54.501 | −14.810 | 28.380 | 1.00 | 53.96 | N | |
| ATOM | 1508 | CZ | ARG | A | 328 | 54.655 | −14.481 | 29.664 | 1.00 | 55.35 | C | |
| ATOM | 1509 | NH1 | ARG | A | 328 | 53.687 | −14.670 | 30.581 | 1.00 | 57.04 | N | |
| ATOM | 1512 | NH2 | ARG | A | 328 | 55.839 | −13.993 | 30.042 | 1.00 | 54.89 | N | |
| ATOM | 1515 | C | ARG | A | 328 | 51.281 | −17.120 | 23.586 | 1.00 | 51.26 | C | |
| ATOM | 1516 | O | ARG | A | 328 | 51.868 | −18.100 | 23.195 | 1.00 | 50.00 | O | |
| ATOM | 1518 | N | GLU | A | 329 | 49.954 | −17.017 | 23.635 | 1.00 | 52.51 | N | |
| ATOM | 1519 | CA | GLU | A | 329 | 49.023 | −18.098 | 23.387 | 1.00 | 53.25 | C | |
| ATOM | 1521 | CB | GLU | A | 329 | 47.780 | −17.993 | 24.310 | 1.00 | 53.62 | C | |
| ATOM | 1524 | CG | GLU | A | 329 | 47.975 | −18.613 | 25.700 | 1.00 | 57.15 | C | |
| ATOM | 1527 | CD | GLU | A | 329 | 47.579 | −17.705 | 26.888 | 1.00 | 63.27 | C | |
| ATOM | 1528 | OE1 | GLU | A | 329 | 46.461 | −17.105 | 26.890 | 1.00 | 66.10 | O | |
| ATOM | 1529 | OE2 | GLU | A | 329 | 48.402 | −17.583 | 27.849 | 1.00 | 67.56 | O | |
| ATOM | 1530 | C | GLU | A | 329 | 48.602 | −18.156 | 21.936 | 1.00 | 53.37 | C | |
| ATOM | 1531 | O | GLU | A | 329 | 48.052 | −19.192 | 21.536 | 1.00 | 54.14 | O | |
| ATOM | 1533 | N | CYS | A | 330 | 48.875 | −17.116 | 21.136 | 1.00 | 53.25 | N | |
| ATOM | 1534 | CA | CYS | A | 330 | 48.327 | −17.031 | 19.766 | 1.00 | 53.54 | C | |
| ATOM | 1536 | CB | CYS | A | 330 | 48.265 | −15.601 | 19.268 | 1.00 | 53.40 | C | |
| ATOM | 1539 | SG | CYS | A | 330 | 49.873 | −15.008 | 18.732 | 1.00 | 57.08 | S | |
| ATOM | 1541 | C | CYS | A | 330 | 49.058 | −17.852 | 18.698 | 1.00 | 53.17 | C | |
| ATOM | 1542 | O | CYS | A | 330 | 50.240 | −18.190 | 18.846 | 1.00 | 52.64 | O | |
| ATOM | 1544 | N | ASN | A | 331 | 48.277 | −18.169 | 17.653 | 1.00 | 53.38 | N | |
| ATOM | 1545 | CA | ASN | A | 331 | 48.710 | −18.569 | 16.285 | 1.00 | 52.93 | C | |
| ATOM | 1547 | CB | ASN | A | 331 | 47.486 | −19.166 | 15.557 | 1.00 | 52.32 | C | |
| ATOM | 1550 | CG | ASN | A | 331 | 47.676 | −19.381 | 14.070 | 1.00 | 51.85 | C | |
| ATOM | 1551 | OD1 | ASN | A | 331 | 48.167 | −18.533 | 13.346 | 1.00 | 52.00 | O | |
| ATOM | 1552 | ND2 | ASN | A | 331 | 47.187 | −20.507 | 13.594 | 1.00 | 52.93 | N | |
| ATOM | 1555 | C | ASN | A | 331 | 49.307 | −17.330 | 15.535 | 1.00 | 53.24 | C | |
| ATOM | 1556 | O | ASN | A | 331 | 48.568 | −16.404 | 15.117 | 1.00 | 52.88 | O | |
| ATOM | 1558 | N | ARG | A | 332 | 50.626 | −17.380 | 15.301 | 1.00 | 53.17 | N | |
| ATOM | 1559 | CA | ARO | A | 332 | 51.414 | −16.231 | 14.795 | 1.00 | 52.68 | C | |
| ATOM | 1561 | CB | ARG | A | 332 | 52.895 | −16.413 | 15.158 | 1.00 | 52.67 | C | |
| ATOM | 1564 | CG | ARG | A | 332 | 53.163 | −16.648 | 16.633 | 1.00 | 51.10 | C | |
| ATOM | 1567 | CD | ARG | A | 332 | 54.590 | −16.702 | 16.951 | 1.00 | 49.69 | C | |
| ATOM | 1570 | NE | ARG | A | 332 | 54.852 | −16.927 | 18.369 | 1.00 | 48.10 | N | |
| ATOM | 1572 | CZ | ARG | A | 332 | 56.074 | −17.005 | 18.907 | 1.00 | 45.55 | C | |
| ATOM | 1573 | NH1 | ARG | A | 332 | 57.177 | −16.893 | 18.153 | 1.00 | 46.29 | N | |
| ATOM | 1576 | NH2 | ARG | A | 332 | 56.197 | −17.186 | 20.208 | 1.00 | 42.43 | N | |
| ATOM | 1579 | C | ARG | A | 332 | 51.324 | −16.002 | 13.294 | 1.00 | 53.91 | C | |
| ATOM | 1580 | O | ARG | A | 332 | 51.780 | −14.968 | 12.781 | 1.00 | 53.57 | O | |
| ATOM | 1582 | N | GLN | A | 333 | 50.769 | −16.986 | 12.590 | 1.00 | 55.31 | N | |
| ATOM | 1583 | CA | GLN | A | 333 | 50.434 | −16.842 | 11.166 | 1.00 | 56.72 | C | |
| ATOM | 1585 | CB | GLN | A | 333 | 49.995 | −18.169 | 10.503 | 1.00 | 57.03 | C | |
| ATOM | 1588 | CG | GLN | A | 333 | 50.601 | −19.504 | 11.021 | 1.00 | 59.84 | C | |
| ATOM | 1591 | CD | GLN | A | 333 | 52.087 | −19.444 | 11.422 | 1.00 | 63.22 | C | |
| ATOM | 1592 | OE1 | GLN | A | 333 | 52.891 | −18.682 | 10.859 | 1.00 | 64.83 | O | |
| ATOM | 1593 | NE2 | GLN | A | 333 | 52.433 | −20.229 | 12.427 | 1.00 | 64.99 | N | |
| ATOM | 1596 | C | GLN | A | 333 | 49.316 | −15.784 | 11.021 | 1.00 | 57.10 | C | |
| ATOM | 1597 | O | GLN | A | 333 | 49.374 | −14.939 | 10.124 | 1.00 | 57.29 | O | |
| ATOM | 1599 | N | GLU | A | 334 | 48.333 | −15.830 | 11.924 | 1.00 | 57.26 | N | |
| ATOM | 1600 | CA | GLU | A | 334 | 47.282 | −14.841 | 11.969 | 1.00 | 57.93 | C | |
| ATOM | 1602 | CB | GLU | A | 334 | 46.093 | −15.439 | 12.703 | 1.00 | 57.81 | C | |
| ATOM | 1605 | CG | GLU | A | 334 | 44.824 | −14.623 | 12.556 | 1.00 | 59.79 | C | |
| ATOM | 1608 | CD | GLU | A | 334 | 43.711 | −15.160 | 13.419 | 1.00 | 60.61 | C | |
| ATOM | 1609 | OE1 | GLU | A | 334 | 43.301 | −16.310 | 13.150 | 1.00 | 65.79 | O | |
| ATOM | 1610 | OE2 | GLU | A | 334 | 43.254 | −14.444 | 14.348 | 1.00 | 64.99 | O | |
| ATOM | 1611 | C | GLU | A | 334 | 47.765 | −13.495 | 12.616 | 1.00 | 57.92 | C | |
| ATOM | 1612 | O | GLU | A | 334 | 47.579 | −12.405 | 12.036 | 1.00 | 57.91 | O | |
| ATOM | 1614 | N | VAL | A | 335 | 48.362 | −13.597 | 13.810 | 1.00 | 57.54 | N | |
| ATOM | 1615 | CA | VAL | A | 335 | 49.011 | −12.481 | 14.520 | 1.00 | 57.11 | C | |
| ATOM | 1617 | CB | VAL | A | 335 | 49.000 | −12.731 | 16.054 | 1.00 | 56.99 | C | |
| ATOM | 1619 | CG1 | VAL | A | 335 | 49.665 | −11.564 | 16.841 | 1.00 | 57.86 | C | |
| ATOM | 1623 | CG2 | VAL | A | 335 | 47.582 | −12.975 | 16.563 | 1.00 | 56.72 | C | |
| ATOM | 1627 | C | VAL | A | 335 | 50.463 | −12.369 | 13.982 | 1.00 | 57.12 | C | |
| ATOM | 1628 | O | VAL | A | 335 | 51.429 | −12.835 | 14.592 | 1.00 | 57.16 | O | |
| ATOM | 1630 | N | ASN | A | 336 | 50.598 | −11.773 | 12.807 | 1.00 | 56.99 | N | |
| ATOM | 1631 | CA | ASN | A | 336 | 51.851 | −11.794 | 12.029 | 1.00 | 56.48 | C | |
| ATOM | 1633 | CB | ASN | A | 336 | 51.544 | −12.181 | 10.561 | 1.00 | 56.72 | C | |
| ATOM | 1636 | CG | ASN | A | 336 | 50.867 | −11.069 | 9.769 | 1.00 | 57.43 | C | |
| ATOM | 1637 | OD1 | ASN | A | 336 | 50.411 | −10.071 | 10.331 | 1.00 | 61.57 | O | |
| ATOM | 1638 | ND2 | ASN | A | 336 | 50.813 | −11.236 | 8.459 | 1.00 | 58.02 | N | |
| ATOM | 1641 | C | ASN | A | 336 | 52.600 | −10.467 | 12.132 | 1.00 | 55.12 | C | |
| ATOM | 1642 | O | ASN | A | 336 | 52.102 | −9.533 | 12.736 | 1.00 | 54.98 | O | |
| ATOM | 1644 | N | ALA | A | 337 | 53.763 | −10.399 | 11.497 | 1.00 | 54.01 | N | |
| ATOM | 1645 | CA | ALA | A | 337 | 54.635 | −9.224 | 11.525 | 1.00 | 53.69 | C | |
| ATOM | 1647 | CB | ALA | A | 337 | 55.698 | −9.338 | 10.440 | 1.00 | 53.52 | C | |
| ATOM | 1651 | C | ALA | A | 337 | 53.901 | −7.876 | 11.405 | 1.00 | 53.41 | C | |
| ATOM | 1652 | O | ALA | A | 337 | 54.078 | −6.979 | 12.231 | 1.00 | 52.79 | O | |
| ATOM | 1654 | N | VAL | A | 338 | 53.053 | −7.759 | 10.396 | 1.00 | 53.44 | N | |
| ATOM | 1655 | CA | VAL | A | 338 | 52.275 | −6.518 | 10.209 | 1.00 | 53.57 | C | |
| ATOM | 1657 | CB | VAL | A | 338 | 51.583 | −6.427 | 8.808 | 1.00 | 53.82 | C | |
| ATOM | 1659 | CG1 | VAL | A | 338 | 50.550 | −7.553 | 8.573 | 1.00 | 53.74 | C | |
| ATOM | 1663 | CG2 | VAL | A | 338 | 50.928 | −5.010 | 8.593 | 1.00 | 53.91 | C | |
| ATOM | 1667 | C | VAL | A | 338 | 51.254 | −6.268 | 11.341 | 1.00 | 52.91 | C | |
| ATOM | 1668 | O | VAL | A | 338 | 51.054 | −5.115 | 11.735 | 1.00 | 52.27 | O | |
| ATOM | 1670 | N | VAL | A | 339 | 50.615 | −7.329 | 11.849 | 1.00 | 52.29 | N | |
| ATOM | 1671 | CA | VAL | A | 339 | 49.711 | −7.161 | 12.985 | 1.00 | 51.79 | C | |
| ATOM | 1673 | CB | VAL | A | 339 | 49.039 | −8.465 | 13.452 | 1.00 | 51.84 | C | |
| ATOM | 1675 | CG1 | VAL | A | 339 | 48.353 | −8.298 | 14.885 | 1.00 | 50.59 | C | |
| ATOM | 1679 | CG2 | VAL | A | 339 | 48.033 | −8.914 | 12.421 | 1.00 | 50.16 | C | |
| ATOM | 1683 | C | VAL | A | 339 | 50.459 | −6.503 | 14.132 | 1.00 | 51.96 | C | |
| ATOM | 1684 | O | VAL | A | 339 | 49.960 | −5.499 | 14.639 | 1.00 | 51.30 | O | |
| ATOM | 1686 | N | LEU | A | 340 | 51.660 | −7.037 | 14.468 | 1.00 | 51.94 | N | |
| ATOM | 1687 | CA | LEU | A | 340 | 52.527 | −6.500 | 15.555 | 1.00 | 51.95 | C | |
| ATOM | 1689 | CB | LEU | A | 340 | 53.866 | −7.233 | 15.651 | 1.00 | 51.64 | C | |
| ATOM | 1692 | CG | LEU | A | 340 | 53.886 | −8.764 | 15.795 | 1.00 | 53.25 | C | |
| ATOM | 1694 | CD1 | LEU | A | 340 | 55.285 | −9.281 | 16.075 | 1.00 | 52.63 | C | |
| ATOM | 1698 | CD2 | LEU | A | 340 | 52.935 | −9.269 | 16.882 | 1.00 | 53.62 | C | |
| ATOM | 1702 | C | LEU | A | 340 | 52.778 | −4.996 | 15.420 | 1.00 | 52.11 | C | |
| ATOM | 1703 | O | LEU | A | 340 | 52.789 | −4.244 | 16.408 | 1.00 | 53.25 | O | |
| ATOM | 1705 | N | LEU | A | 341 | 52.923 | −4.569 | 14.182 | 1.00 | 51.95 | N | |
| ATOM | 1706 | CA | LEU | A | 341 | 53.220 | −3.212 | 13.848 | 1.00 | 52.33 | C | |
| ATOM | 1708 | CB | LEU | A | 341 | 53.711 | −3.173 | 12.375 | 1.00 | 52.64 | C | |
| ATOM | 1711 | CG | LEU | A | 341 | 54.864 | −2.267 | 11.892 | 1.00 | 54.23 | C | |
| ATOM | 1713 | CD1 | LEU | A | 341 | 56.093 | −2.219 | 12.805 | 1.00 | 53.35 | C | |
| ATOM | 1717 | CD2 | LEU | A | 341 | 55.276 | −2.663 | 10.417 | 1.00 | 53.46 | C | |
| ATOM | 1721 | C | LEU | A | 341 | 51.986 | −2.321 | 14.144 | 1.00 | 52.22 | C | |
| ATOM | 1722 | O | LEU | A | 341 | 52.147 | −1.225 | 14.669 | 1.00 | 52.70 | O | |
| ATOM | 1724 | N | TYR | A | 342 | 50.773 | −2.827 | 13.878 | 1.00 | 52.15 | N | |
| ATOM | 1725 | CA | TYR | A | 342 | 49.482 | −2.135 | 14.198 | 1.00 | 51.96 | C | |
| ATOM | 1727 | CB | TYR | A | 342 | 48.299 | −2.945 | 13.630 | 1.00 | 51.65 | C | |
| ATOM | 1730 | CG | TYR | A | 342 | 46.887 | −2.336 | 13.691 | 1.00 | 51.98 | C | |
| ATOM | 1731 | CD1 | TYR | A | 342 | 46.672 | −0.941 | 13.633 | 1.00 | 51.01 | C | |
| ATOM | 1733 | CE1 | TYR | A | 342 | 45.374 | −0.398 | 13.688 | 1.00 | 50.45 | C | |
| ATOM | 1735 | CZ | TYR | A | 342 | 44.279 | −1.244 | 13.754 | 1.00 | 51.02 | C | |
| ATOM | 1736 | OH | TYR | A | 342 | 43.003 | −0.685 | 13.795 | 1.00 | 51.89 | O | |
| ATOM | 1738 | CE2 | TYR | A | 342 | 44.460 | −2.640 | 13.779 | 1.00 | 50.55 | C | |
| ATOM | 1740 | CD2 | TYR | A | 342 | 45.748 | −3.178 | 13.743 | 1.00 | 49.97 | C | |
| ATOM | 1742 | C | TYR | A | 342 | 49.269 | −1.938 | 15.686 | 1.00 | 52.12 | C | |
| ATOM | 1743 | O | TYR | A | 342 | 48.806 | −0.891 | 16.129 | 1.00 | 52.02 | O | |
| ATOM | 1745 | N | MET | A | 343 | 49.665 | −2.945 | 16.453 | 1.00 | 52.44 | N | |
| ATOM | 1746 | CA | MET | A | 343 | 49.497 | −2.944 | 17.905 | 1.00 | 52.85 | C | |
| ATOM | 1748 | CB | MET | A | 343 | 49.816 | −4.314 | 18.488 | 1.00 | 52.17 | C | |
| ATOM | 1751 | CG | MET | A | 343 | 48.860 | −5.347 | 17.896 | 1.00 | 53.59 | C | |
| ATOM | 1754 | SD | MET | A | 343 | 48.691 | −6.887 | 18.680 | 1.00 | 55.71 | S | |
| ATOM | 1755 | CE | MET | A | 343 | 50.344 | −7.441 | 18.756 | 1.00 | 55.23 | C | |
| ATOM | 1759 | C | MET | A | 343 | 50.364 | −1.885 | 18.485 | 1.00 | 52.32 | C | |
| ATOM | 1760 | O | MET | A | 343 | 49.875 | −0.995 | 19.193 | 1.00 | 52.53 | O | |
| ATOM | 1762 | N | ALA | A | 344 | 51.638 | −1.933 | 18.111 | 1.00 | 52.07 | N | |
| ATOM | 1763 | CA | ALA | A | 344 | 52.573 | −0.868 | 18.465 | 1.00 | 51.62 | C | |
| ATOM | 1765 | CB | ALA | A | 344 | 53.951 | −1.153 | 17.878 | 1.00 | 51.90 | C | |
| ATOM | 1769 | C | ALA | A | 344 | 52.081 | 0.516 | 18.037 | 1.00 | 51.28 | C | |
| ATOM | 1770 | O | ALA | A | 344 | 52.253 | 1.469 | 18.782 | 1.00 | 51.72 | O | |
| ATOM | 1772 | N | THR | A | 345 | 51.463 | 0.611 | 16.855 | 1.00 | 50.78 | N | |
| ATOM | 1773 | CA | TER | A | 345 | 50.996 | 1.897 | 16.311 | 1.00 | 50.28 | C | |
| ATOM | 1775 | CB | THR | A | 345 | 50.568 | 1.761 | 14.843 | 1.00 | 50.01 | C | |
| ATOM | 1777 | OG1 | THR | A | 345 | 51.645 | 1.230 | 14.077 | 1.00 | 47.39 | O | |
| ATOM | 1779 | CG2 | THR | A | 345 | 50.318 | 3.164 | 14.233 | 1.00 | 49.91 | C | |
| ATOM | 1783 | C | THR | A | 345 | 49.819 | 2.471 | 17.078 | 1.00 | 49.83 | C | |
| ATOM | 1784 | O | THR | A | 345 | 49.737 | 3.673 | 17.279 | 1.00 | 48.98 | O | |
| ATOM | 1786 | N | GLN | A | 346 | 48.876 | 1.589 | 17.412 | 1.00 | 49.88 | N | |
| ATOM | 1787 | CA | GLN | A | 346 | 47.711 | 1.939 | 18.243 | 1.00 | 49.50 | C | |
| ATOM | 1789 | CB | GLN | A | 346 | 46.809 | 0.714 | 18.472 | 1.00 | 49.19 | C | |
| ATOM | 1792 | CG | GLN | A | 346 | 46.083 | 0.234 | 17.254 | 1.00 | 48.02 | C | |
| ATOM | 1795 | CD | GLN | A | 346 | 45.349 | −1.060 | 17.521 | 1.00 | 48.89 | C | |
| ATOM | 1796 | OE1 | GLN | A | 346 | 44.354 | −1.055 | 18.278 | 1.00 | 47.75 | O | |
| ATOM | 1797 | NE2 | GLN | A | 346 | 45.822 | −2.175 | 16.926 | 1.00 | 44.34 | N | |
| ATOM | 1800 | C | GLN | A | 346 | 48.190 | 2.473 | 19.582 | 1.00 | 49.15 | C | |
| ATOM | 1801 | O | GLN | A | 346 | 47.789 | 3.548 | 20.018 | 1.00 | 49.02 | O | |
| ATOM | 1803 | N | ILE | A | 347 | 49.092 | 1.726 | 20.197 | 1.00 | 48.85 | N | |
| ATOM | 1804 | CA | ILE | A | 347 | 49.515 | 2.048 | 21.528 | 1.00 | 49.44 | C | |
| ATOM | 1806 | CB | ILE | A | 347 | 50.322 | 0.900 | 22.152 | 1.00 | 49.65 | C | |
| ATOM | 1808 | CG1 | ILE | A | 347 | 49.368 | −0.298 | 22.352 | 1.00 | 49.70 | C | |
| ATOM | 1811 | CD1 | ILE | A | 347 | 50.031 | −1.577 | 22.599 | 1.00 | 50.76 | C | |
| ATOM | 1815 | CG2 | ILE | A | 347 | 50.904 | 1.315 | 23.523 | 1.00 | 49.56 | C | |
| ATOM | 1819 | C | ILE | A | 347 | 50.224 | 3.382 | 21.536 | 1.00 | 49.59 | C | |
| ATOM | 1820 | O | ILE | A | 347 | 49.936 | 4.202 | 22.404 | 1.00 | 49.17 | O | |
| ATOM | 1822 | N | SER | A | 348 | 51.078 | 3.626 | 20.542 | 1.00 | 49.74 | N | |
| ATOM | 1823 | CA | SER | A | 348 | 51.701 | 4.941 | 20.389 | 1.00 | 50.09 | C | |
| ATOM | 1825 | CB | SER | A | 348 | 52.726 | 4.954 | 19.243 | 1.00 | 49.95 | C | |
| ATOM | 1828 | OG | SER | A | 348 | 52.279 | 4.291 | 18.081 | 1.00 | 49.56 | O | |
| ATOM | 1830 | C | SER | A | 348 | 50.652 | 6.065 | 20.247 | 1.00 | 50.75 | C | |
| ATOM | 1831 | O | SER | A | 348 | 50.768 | 7.117 | 20.881 | 1.00 | 51.02 | O | |
| ATOM | 1833 | N | SER | A | 349 | 49.595 | 5.816 | 19.485 | 1.00 | 51.78 | N | |
| ATOM | 1834 | CA | SER | A | 349 | 48.547 | 6.817 | 19.294 | 1.00 | 52.48 | C | |
| ATOM | 1836 | CB | SER | A | 349 | 47.418 | 6.287 | 18.444 | 1.00 | 52.11 | C | |
| ATOM | 1839 | OG | SER | A | 349 | 46.588 | 7.370 | 18.164 | 1.00 | 54.26 | O | |
| ATOM | 1841 | C | SER | A | 349 | 47.977 | 7.313 | 20.620 | 1.00 | 52.81 | C | |
| ATOM | 1842 | O | SER | A | 349 | 47.901 | 8.524 | 20.859 | 1.00 | 52.83 | O | |
| ATOM | 1844 | N | ALA | A | 350 | 47.619 | 6.359 | 21.476 | 1.00 | 53.39 | N | |
| ATOM | 1845 | CA | ALA | A | 350 | 47.093 | 6.663 | 22.822 | 1.00 | 53.78 | C | |
| ATOM | 1847 | CB | ALA | A | 350 | 46.609 | 5.393 | 23.480 | 1.00 | 53.95 | C | |
| ATOM | 1851 | C | ALA | A | 350 | 48.063 | 7.372 | 23.779 | 1.00 | 53.96 | C | |
| ATOM | 1852 | O | ALA | A | 350 | 47.615 | 8.035 | 24.722 | 1.00 | 54.73 | O | |
| ATOM | 1854 | N | MET | A | 351 | 49.366 | 7.180 | 23.565 | 1.00 | 53.72 | N | |
| ATOM | 1855 | CA | MET | A | 351 | 50.408 | 7.732 | 24.413 | 1.00 | 53.43 | C | |
| ATOM | 1857 | CB | MET | A | 351 | 51.612 | 6.827 | 24.385 | 1.00 | 53.33 | C | |
| ATOM | 1860 | CG | MET | A | 351 | 51.393 | 5.506 | 25.060 | 1.00 | 53.80 | C | |
| ATOM | 1863 | SD | MET | A | 351 | 50.982 | 5.577 | 26.819 | 1.00 | 55.30 | S | |
| ATOM | 1864 | CE | MET | A | 351 | 52.240 | 6.513 | 27.585 | 1.00 | 57.96 | C | |
| ATOM | 1868 | C | MET | A | 351 | 50.811 | 9.131 | 23.992 | 1.00 | 53.93 | C | |
| ATOM | 1869 | O | MET | A | 351 | 51.166 | 9.960 | 24.831 | 1.00 | 52.90 | O | |
| ATOM | 1871 | N | GLU | A | 352 | 50.778 | 9.390 | 22.694 | 1.00 | 54.74 | N | |
| ATOM | 1872 | CA | GLU | A | 352 | 50.848 | 10.756 | 22.200 | 1.00 | 56.24 | C | |
| ATOM | 1874 | CB | GLU | A | 352 | 50.792 | 10.751 | 20.687 | 1.00 | 56.27 | C | |
| ATOM | 1877 | CG | GLU | A | 352 | 50.817 | 12.126 | 20.017 | 1.00 | 57.41 | C | |
| ATOM | 1880 | CD | GLU | A | 352 | 50.642 | 12.033 | 18.512 | 1.00 | 58.76 | C | |
| ATOM | 1881 | OE1 | GLU | A | 352 | 50.134 | 10.976 | 18.034 | 1.00 | 64.72 | O | |
| ATOM | 1882 | OE2 | GLU | A | 352 | 51.018 | 13.003 | 17.801 | 1.00 | 60.35 | O | |
| ATOM | 1883 | C | GLU | A | 352 | 49.689 | 11.606 | 22.745 | 1.00 | 56.18 | C | |
| ATOM | 1884 | O | GLU | A | 352 | 49.882 | 12.808 | 23.011 | 1.00 | 56.18 | O | |
| ATOM | 1886 | N | TYR | A | 353 | 48.502 | 10.990 | 22.858 | 1.00 | 56.38 | N | |
| ATOM | 1887 | CA | TYR | A | 353 | 47.310 | 11.639 | 23.426 | 1.00 | 56.45 | C | |
| ATOM | 1889 | CB | TYR | A | 353 | 46.060 | 10.745 | 23.266 | 1.00 | 56.57 | C | |
| ATOM | 1892 | CG | TYR | A | 353 | 44.797 | 11.231 | 23.969 | 1.00 | 56.99 | C | |
| ATOM | 1893 | CD1 | TYR | A | 353 | 44.023 | 12.288 | 23.447 | 1.00 | 57.49 | C | |
| ATOM | 1895 | CE1 | TYR | A | 353 | 42.856 | 12.744 | 24.111 | 1.00 | 56.27 | C | |
| ATOM | 1897 | CZ | TYR | A | 353 | 42.457 | 12.124 | 25.289 | 1.00 | 56.81 | C | |
| ATOM | 1898 | OH | TYR | A | 353 | 41.318 | 12.536 | 25.955 | 1.00 | 57.14 | O | |
| ATOM | 1900 | CE2 | TYR | A | 353 | 43.201 | 11.067 | 25.813 | 1.00 | 56.88 | C | |
| ATOM | 1902 | CD2 | TYR | A | 353 | 44.357 | 10.625 | 25.157 | 1.00 | 57.07 | C | |
| ATOM | 1904 | C | TYR | A | 353 | 47.599 | 11.986 | 24.880 | 1.00 | 56.93 | C | |
| ATOM | 1905 | O | TYR | A | 353 | 47.515 | 13.148 | 25.269 | 1.00 | 56.46 | O | |
| ATOM | 1907 | N | LEU | A | 354 | 48.007 | 10.984 | 25.659 | 1.00 | 57.62 | N | |
| ATOM | 1908 | CA | LEU | A | 354 | 48.389 | 11.199 | 27.063 | 1.00 | 58.03 | C | |
| ATOM | 1910 | CB | LEU | A | 354 | 48.769 | 9.875 | 27.720 | 1.00 | 57.98 | C | |
| ATOM | 1913 | CG | LEU | A | 354 | 47.692 | 9.076 | 28.468 | 1.00 | 59.37 | C | |
| ATOM | 1915 | CD1 | LEU | A | 354 | 46.305 | 9.179 | 27.855 | 1.00 | 61.13 | C | |
| ATOM | 1919 | CD2 | LEU | A | 354 | 48.087 | 7.600 | 28.598 | 1.00 | 58.39 | C | |
| ATOM | 1923 | C | LEU | A | 354 | 49.507 | 12.224 | 27.222 | 1.00 | 58.26 | C | |
| ATOM | 1924 | O | LEU | A | 354 | 49.441 | 13.050 | 28.112 | 1.00 | 58.67 | O | |
| ATOM | 1926 | N | GLU | A | 355 | 50.510 | 12.164 | 26.347 | 1.00 | 58.64 | N | |
| ATOM | 1927 | CA | GLU | A | 355 | 51.601 | 13.154 | 26.278 | 1.00 | 58.85 | C | |
| ATOM | 1929 | CB | GLU | A | 355 | 52.604 | 12.715 | 25.190 | 1.00 | 58.98 | C | |
| ATOM | 1932 | CG | GLU | A | 355 | 53.784 | 13.631 | 24.847 | 1.00 | 59.78 | C | |
| ATOM | 1935 | CD | GLU | A | 355 | 54.477 | 13.209 | 23.549 | 1.00 | 61.24 | C | |
| ATOM | 1936 | OE1 | GLU | A | 355 | 53.920 | 13.513 | 22.451 | 1.00 | 63.60 | O | |
| ATOM | 1937 | OE2 | GLU | A | 355 | 55.555 | 12.548 | 23.629 | 1.00 | 63.93 | O | |
| ATOM | 1938 | C | GLU | A | 355 | 51.112 | 14.601 | 26.027 | 1.00 | 58.65 | C | |
| ATOM | 1939 | O | GLU | A | 355 | 51.752 | 15.556 | 26.497 | 1.00 | 57.91 | O | |
| ATOM | 1941 | N | LYS | A | 356 | 50.008 | 14.747 | 25.283 | 1.00 | 58.75 | N | |
| ATOM | 1942 | CA | LYS | A | 356 | 49.436 | 16.075 | 24.938 | 1.00 | 59.18 | C | |
| ATOM | 1944 | CB | LYS | A | 356 | 48.437 | 16.005 | 23.758 | 1.00 | 58.87 | C | |
| ATOM | 1947 | CG | LYS | A | 356 | 48.797 | 16.898 | 22.576 | 1.00 | 61.02 | C | |
| ATOM | 1950 | CD | LYS | A | 356 | 47.563 | 17.543 | 21.890 | 1.00 | 62.28 | C | |
| ATOM | 1953 | CE | LYS | A | 356 | 46.584 | 16.535 | 21.232 | 1.00 | 65.16 | C | |
| ATOM | 1956 | NZ | LYS | A | 356 | 45.452 | 17.270 | 20.510 | 1.00 | 64.83 | N | |
| ATOM | 1960 | C | LYS | A | 356 | 48.750 | 16.656 | 26.165 | 1.00 | 58.24 | C | |
| ATOM | 1961 | O | LYS | A | 356 | 48.938 | 17.826 | 26.495 | 1.00 | 57.93 | O | |
| ATOM | 1963 | N | LYS | A | 357 | 47.981 | 15.808 | 26.842 | 1.00 | 57.84 | N | |
| ATOM | 1964 | CA | LYS | A | 357 | 47.324 | 16.152 | 28.109 | 1.00 | 57.62 | C | |
| ATOM | 1966 | CB | LYS | A | 357 | 46.173 | 15.155 | 28.426 | 1.00 | 57.35 | C | |
| ATOM | 1969 | CG | LYS | A | 357 | 45.115 | 14.990 | 27.333 | 1.00 | 58.70 | C | |
| ATOM | 1972 | CD | LYS | A | 357 | 43.715 | 14.649 | 27.915 | 1.00 | 59.47 | C | |
| ATOM | 1975 | CE | LYS | A | 357 | 42.817 | 15.920 | 28.167 | 1.00 | 61.35 | C | |
| ATOM | 1978 | NZ | LYS | A | 357 | 41.740 | 15.731 | 29.238 | 1.00 | 61.43 | N | |
| ATOM | 1982 | C | LYS | A | 357 | 48.279 | 16.235 | 29.323 | 1.00 | 56.77 | C | |
| ATOM | 1983 | O | LYS | A | 357 | 47.819 | 16.557 | 30.398 | 1.00 | 56.59 | O | |
| ATOM | 1985 | N | ASN | A | 358 | 49.570 | 15.936 | 29.164 | 1.00 | 56.33 | N | |
| ATOM | 1986 | CA | ASN | A | 358 | 50.543 | 15.848 | 30.274 | 1.00 | 56.66 | C | |
| ATOM | 1988 | CB | ASN | A | 358 | 50.749 | 17.231 | 30.912 | 1.00 | 56.15 | C | |
| ATOM | 1991 | CG | ASN | A | 358 | 52.137 | 17.392 | 31.539 | 1.00 | 56.69 | C | |
| ATOM | 1992 | OD1 | ASN | A | 358 | 53.178 | 17.171 | 30.886 | 1.00 | 58.14 | O | |
| ATOM | 1993 | ND2 | ASN | A | 358 | 52.159 | 17.745 | 32.816 | 1.00 | 54.39 | N | |
| ATOM | 1996 | C | ASN | A | 358 | 50.313 | 14.731 | 31.354 | 1.00 | 56.66 | C | |
| ATOM | 1997 | O | ASN | A | 358 | 50.644 | 14.895 | 32.555 | 1.00 | 55.97 | O | |
| ATOM | 1999 | N | PHE | A | 359 | 49.802 | 13.585 | 30.897 | 1.00 | 56.72 | N | |
| ATOM | 2000 | CA | PHE | A | 359 | 49.605 | 12.415 | 31.746 | 1.00 | 57.52 | C | |
| ATOM | 2002 | CB | PHE | A | 359 | 48.360 | 11.629 | 31.299 | 1.00 | 58.78 | C | |
| ATOM | 2005 | CG | PHE | A | 359 | 47.094 | 12.192 | 31.820 | 1.00 | 59.04 | C | |
| ATOM | 2006 | CD1 | PHE | A | 359 | 46.622 | 13.418 | 31.332 | 1.00 | 62.61 | C | |
| ATOM | 2008 | CE1 | PHE | A | 359 | 45.400 | 14.032 | 31.831 | 1.00 | 61.37 | C | |
| ATOM | 2010 | CZ | PHE | A | 359 | 44.687 | 13.399 | 32.845 | 1.00 | 61.22 | C | |
| ATOM | 2012 | CE2 | PHE | A | 359 | 45.174 | 12.135 | 33.347 | 1.00 | 62.29 | C | |
| ATOM | 2014 | CD2 | PHE | A | 359 | 46.371 | 11.545 | 32.818 | 1.00 | 60.64 | C | |
| ATOM | 2016 | C | PHE | A | 359 | 50.845 | 11.515 | 31.702 | 1.00 | 57.59 | C | |
| ATOM | 2017 | O | PHE | A | 359 | 51.493 | 11.460 | 30.685 | 1.00 | 58.75 | O | |
| ATOM | 2019 | N | ILE | A | 360 | 51.192 | 10.839 | 32.787 | 1.00 | 56.99 | N | |
| ATOM | 2020 | CA | ILE | A | 360 | 52.056 | 9.657 | 32.690 | 1.00 | 57.39 | C | |
| ATOM | 2022 | CB | ILE | A | 360 | 53.287 | 9.660 | 33.655 | 1.00 | 57.76 | C | |
| ATOM | 2024 | CG1 | ILE | A | 360 | 52.897 | 9.824 | 35.137 | 1.00 | 58.11 | C | |
| ATOM | 2027 | CD1 | ILE | A | 360 | 54.060 | 9.883 | 36.079 | 1.00 | 58.13 | C | |
| ATOM | 2031 | CG2 | ILE | A | 360 | 54.282 | 10.703 | 33.229 | | 1.00 | 57.51 | C |
| ATOM | 2035 | C | ILE | A | 360 | 51.153 | 8.527 | 33.012 | 1.00 | 56.96 | C | |
| ATOM | 2036 | O | ILE | A | 360 | 50.271 | 8.718 | 33.828 | 1.00 | 57.32 | O | |
| ATOM | 2038 | N | HIS | A | 361 | 51.325 | 7.394 | 32.335 | 1.00 | 56.59 | N | |
| ATOM | 2039 | CA | HIS | A | 361 | 50.718 | 6.107 | 32.727 | 1.00 | 56.34 | C | |
| ATOM | 2041 | CB | HIS | A | 361 | 50.863 | 5.060 | 31.608 | 1.00 | 56.14 | C | |
| ATOM | 2044 | CG | HIS | A | 361 | 49.999 | 3.859 | 31.797 | 1.00 | 54.84 | C | |
| ATOM | 2045 | ND1 | HIS | A | 361 | 50.466 | 2.674 | 32.310 | 1.00 | 54.23 | N | |
| ATOM | 2047 | CE1 | HIS | A | 361 | 49.473 | 1.810 | 32.405 | 1.00 | 53.40 | C | |
| ATOM | 2049 | NE2 | HIS | A | 361 | 48.380 | 2.393 | 31.972 | 1.00 | 54.30 | N | |
| ATOM | 2051 | CD2 | HIS | A | 361 | 48.675 | 3.683 | 31.606 | 1.00 | 54.59 | C | |
| ATOM | 2053 | C | HIS | A | 361 | 51.431 | 5.555 | 33.938 | 1.00 | 57.04 | C | |
| ATOM | 2054 | O | HIS | A | 361 | 50.811 | 5.006 | 34.868 | 1.00 | 56.47 | O | |
| ATOM | 2056 | N | ARG | A | 362 | 52.764 | 5.630 | 33.873 | 1.00 | 58.13 | N | |
| ATOM | 2057 | CA | ARG | A | 362 | 53.670 | 5.149 | 34.936 | 1.00 | 59.12 | C | |
| ATOM | 2059 | CB | ARG | A | 362 | 53.408 | 5.910 | 36.262 | 1.00 | 59.96 | C | |
| ATOM | 2062 | CG | ARG | A | 362 | 54.630 | 6.418 | 37.014 | 1.00 | 61.64 | C | |
| ATOM | 2065 | CD | ARG | A | 362 | 54.472 | 6.384 | 38.571 | 1.00 | 62.77 | C | |
| ATOM | 2068 | NE | ARG | A | 362 | 53.174 | 6.872 | 39.071 | 1.00 | 63.86 | N | |
| ATOM | 2070 | CZ | ARG | A | 362 | 52.809 | 6.857 | 40.363 | 1.00 | 64.88 | C | |
| ATOM | 2071 | NH1 | ARG | A | 362 | 53.623 | 6.402 | 41.314 | 1.00 | 66.44 | N | |
| ATOM | 2074 | NH2 | ARG | A | 362 | 51.624 | 7.340 | 40.729 | 1.00 | 65.03 | N | |
| ATOM | 2077 | C | ARG | A | 362 | 53.670 | 3.619 | 35.170 | 1.00 | 57.97 | C | |
| ATOM | 2078 | O | ARG | A | 362 | 54.380 | 3.199 | 36.039 | 1.00 | 58.27 | O | |
| ATOM | 2080 | N | ASP | A | 363 | 52.922 | 2.812 | 34.401 | 1.00 | 57.45 | N | |
| ATOM | 2081 | CA | ASP | A | 363 | 52.996 | 1.315 | 34.429 | 1.00 | 57.10 | C | |
| ATOM | 2083 | CB | ASP | A | 363 | 52.150 | 0.679 | 35.577 | 1.00 | 56.55 | C | |
| ATOM | 2086 | CG | ASP | A | 363 | 52.383 | −0.845 | 35.721 | 1.00 | 58.60 | C | |
| ATOM | 2087 | OD1 | ASP | A | 363 | 53.440 | −1.365 | 35.254 | 1.00 | 62.48 | O | |
| ATOM | 2088 | OD2 | ASP | A | 363 | 51.548 | −1.632 | 36.226 | 1.00 | 60.76 | O | |
| ATOM | 2089 | C | ASP | A | 363 | 52.651 | 0.684 | 33.036 | 1.00 | 55.60 | C | |
| ATOM | 2090 | O | ASP | A | 363 | 51.767 | −0.155 | 32.896 | 1.00 | 56.32 | O | |
| ATOM | 2092 | N | LEU | A | 364 | 53.387 | 1.082 | 32.019 | 1.00 | 53.90 | N | |
| ATOM | 2093 | CA | LEU | A | 364 | 53.119 | 0.660 | 30.656 | 1.00 | 53.22 | C | |
| ATOM | 2095 | CB | LEU | A | 364 | 53.691 | 1.697 | 29.732 | 1.00 | 52.89 | C | |
| ATOM | 2098 | CG | LEU | A | 364 | 53.155 | 1.663 | 28.334 | 1.00 | 54.86 | C | |
| ATOM | 2100 | CD1 | LEU | A | 364 | 51.636 | 1.946 | 28.288 | 1.00 | 55.65 | C | |
| ATOM | 2104 | CD2 | LEU | A | 364 | 53.947 | 2.720 | 27.538 | 1.00 | 57.12 | C | |
| ATOM | 2108 | C | LEU | A | 364 | 53.776 | −0.687 | 30.355 | 1.00 | 52.95 | C | |
| ATOM | 2109 | O | LEU | A | 364 | 54.998 | −0.830 | 30.537 | 1.00 | 54.77 | O | |
| ATOM | 2111 | N | ALA | A | 365 | 53.001 | −1.662 | 29.899 | 1.00 | 50.88 | N | |
| ATOM | 2112 | CA | ALA | A | 365 | 53.495 | −3.011 | 29.711 | 1.00 | 49.55 | C | |
| ATOM | 2114 | CB | ALA | A | 365 | 53.922 | −3.589 | 31.036 | 1.00 | 49.24 | C | |
| ATOM | 2118 | C | ALA | A | 365 | 52.452 | −3.937 | 29.063 | 1.00 | 49.17 | C | |
| ATOM | 2119 | O | ALA | A | 365 | 51.256 | −3.741 | 29.207 | 1.00 | 47.53 | O | |
| ATOM | 2121 | N | ALA | A | 366 | 52.940 | −4.996 | 28.425 | 1.00 | 49.72 | N | |
| ATOM | 2122 | CA | ALA | A | 366 | 52.118 | −5.946 | 27.658 | 1.00 | 50.24 | C | |
| ATOM | 2124 | CB | ALA | A | 366 | 52.953 | −7.095 | 27.202 | 1.00 | 49.81 | C | |
| ATOM | 2128 | C | ALA | A | 366 | 50.929 | −6.452 | 28.453 | 1.00 | 50.59 | C | |
| ATOM | 2129 | O | ALA | A | 366 | 49.812 | −6.330 | 27.982 | 1.00 | 50.28 | O | |
| ATOM | 2131 | N | ARG | A | 367 | 51.185 | −6.935 | 29.680 | 1.00 | 51.46 | N | |
| ATOM | 2132 | CA | ARG | A | 367 | 50.136 | −7.314 | 30.660 | 1.00 | 52.33 | C | |
| ATOM | 2134 | CB | ARG | A | 367 | 50.773 | −7.718 | 31.988 | 1.00 | 52.44 | C | |
| ATOM | 2137 | CG | ARG | A | 367 | 51.368 | −6.589 | 32.796 | 1.00 | 53.50 | C | |
| ATOM | 2140 | CD | ARG | A | 367 | 52.072 | −6.977 | 34.119 | 1.00 | 55.16 | C | |
| ATOM | 2143 | NE | ARG | A | 367 | 52.654 | −5.752 | 34.715 | 1.00 | 57.55 | N | |
| ATOM | 2145 | CZ | ARG | A | 367 | 53.898 | −5.232 | 34.490 | 1.00 | 59.68 | C | |
| ATOM | 2146 | NH1 | ARG | A | 367 | 54.814 | −5.846 | 33.717 | 1.00 | 59.88 | N | |
| ATOM | 2149 | NH2 | ARG | A | 367 | 54.238 | −4.072 | 35.086 | 1.00 | 59.93 | N | |
| ATOM | 2152 | C | ARG | A | 367 | 49.023 | −6.295 | 30.982 | 1.00 | 52.96 | C | |
| ATOM | 2153 | O | ARG | A | 367 | 47.953 | −6.699 | 31.403 | 1.00 | 53.67 | O | |
| ATOM | 2155 | N | ASN | A | 368 | 49.318 | −4.995 | 30.866 | 1.00 | 53.77 | N | |
| ATOM | 2156 | CA | ASN | A | 368 | 48.353 | −3.882 | 31.064 | 1.00 | 54.07 | C | |
| ATOM | 2158 | CB | ASN | A | 368 | 49.056 | −2.722 | 31.804 | 1.00 | 54.24 | C | |
| ATOM | 2161 | CG | ASN | A | 368 | 49.390 | −3.086 | 33.234 | 1.00 | 56.11 | C | |
| ATOM | 2162 | OD1 | ASN | A | 368 48.635 | −3.843 | 33.883 | 1.00 | 58.47 | O | | |
| ATOM | 2163 | ND2 | ASN | A | 368 50.520 | −2.576 | 33.744 | 1.00 | 56.09 | N | | |
| ATOM | 2166 | C | ASN | A | 368 | 47.665 | −3.363 | 29.782 | 1.00 | 54.37 | C | |
| ATOM | 2167 | O | ASN | A | 368 | 46.982 | −2.329 | 29.810 | 1.00 | 53.43 | O | |
| ATOM | 2169 | N | CYS | A | 369 | 47.852 | −4.087 | 28.671 | 1.00 | 54.80 | N | |
| ATOM | 2170 | CA | CYS | A | 369 | 47.190 | −3.807 | 27.400 | 1.00 | 55.20 | C | |
| ATOM | 2172 | CB | CYS | A | 369 | 48.172 | −3.844 | 26.232 | 1.00 | 55.73 | C | |
| ATOM | 2175 | SG | CYS | A | 369 | 49.292 | −2.416 | 26.161 | 1.00 | 58.82 | S | |
| ATOM | 2177 | C | CYS | A | 369 | 46.173 | −4.873 | 27.201 | 1.00 | 54.94 | C | |
| ATOM | 2178 | O | CYS | A | 369 | 46.459 | −6.017 | 27.462 | 1.00 | 54.51 | O | |
| ATOM | 2180 | N | LEU | A | 370 | 45.009 | −4.479 | 26.711 | 1.00 | 55.57 | N | |
| ATOM | 2181 | CA | LEU | A | 370 | 43.898 | −5.367 | 26.482 | 1.00 | 56.67 | C | |
| ATOM | 2183 | CB | LEU | A | 370 | 42.702 | −4.891 | 27.287 | 1.00 | 56.78 | C | |
| ATOM | 2186 | CC | LEU | A | 370 | 42.893 | −5.112 | 28.791 | 1.00 | 55.78 | C | |
| ATOM | 2188 | CD1 | LEU | A | 370 | 41.825 | −4.348 | 29.533 | 1.00 | 55.28 | C | |
| ATOM | 2192 | CD2 | LEU | A | 370 | 42.874 | −6.579 | 29.176 | 1.00 | 54.67 | C | |
| ATOM | 2196 | C | LEU | A | 370 | 43.563 | −5.419 | 24.998 | 1.00 | 57.88 | C | |
| ATOM | 2197 | O | LEU | A | 370 | 43.827 | −4.454 | 24.282 | 1.00 | 57.65 | O | |
| ATOM | 2199 | N | VAL | A | 371 | 42.978 | −6.545 | 24.568 | 1.00 | 59.20 | N | |
| ATOM | 2200 | CA | VAL | A | 371 | 42.690 | −6.836 | 23.152 | 1.00 | 60.77 | C | |
| ATOM | 2202 | CB | VAL | A | 371 | 43.548 | −8.034 | 22.693 | 1.00 | 61.16 | C | |
| ATOM | 2204 | CC1 | VAL | A | 371 43.586 | −8.163 | 21.135 | 1.00 | 60.80 | C | | |
| ATOM | 2208 | CG2 | VAL | A | 371 44.995 | −7.922 | 23.261 | 1.00 | 61.81 | C | | |
| ATOM | 2212 | C | VAL | A | 371 | 41.186 | −7.127 | 22.837 | 1.00 | 61.14 | C | |
| ATOM | 2213 | O | VAL | A | 371 | 40.486 | −7.813 | 23.604 | 1.00 | 61.52 | O | |
| ATOM | 2215 | N | GLY | A | 372 | 40.717 | −6.602 | 21.708 | 1.00 | 61.60 | N | |
| ATOM | 2216 | CA | GLY | A | 372 | 39.396 | −6.941 | 21.145 | 1.00 | 62.33 | C | |
| ATOM | 2219 | C | GLY | A | 372 | 39.422 | −7.654 | 19.791 | 1.00 | 63.36 | C | |
| ATOM | 2220 | O | GLY | A | 372 | 40.482 | −8.079 | 19.287 | 1.00 | 63.33 | O | |
| ATOM | 2222 | N | GLU | A | 373 | 38.234 | −7.769 | 19.192 | 1.00 | 64.32 | N | |
| ATOM | 2223 | CA | GLU | A | 373 | 38.082 | −8.362 | 17.854 | 1.00 | 64.77 | C | |
| ATOM | 2225 | CB | GLU | A | 373 | 36.594 | −8.462 | 17.420 | 1.00 | 65.14 | C | |
| ATOM | 2228 | CC | GLU | A | 373 | 35.716 | −9.433 | 18.219 | 1.00 | 66.24 | C | |
| ATOM | 2231 | CD | GLU | A | 373 | 36.078 | −10.916 | 18.038 | 1.00 | 69.46 | C | |
| ATOM | 2232 | OE1 | GLU | A | 373 | 36.931 | −11.296 | 17.183 | 1.00 | 70.44 | O | |
| ATOM | 2233 | OE2 | GLU | A | 373 | 35.514 | −11.739 | 18.788 | 1.00 | 72.43 | O | |
| ATOM | 2234 | C | GLU | A | 373 | 38.852 | −7.519 | 16.834 | 1.00 | 64.57 | C | |
| ATOM | 2235 | O | GLU | A | 373 | 38.887 | −6.269 | 16.931 | 1.00 | 64.10 | O | |
| ATOM | 2237 | N | ASN | A | 374 | 39.455 | −8.227 | 15.870 | 1.00 | 64.46 | N | |
| ATOM | 2238 | CA | ASN | A | 374 | 40.229 | −7.625 | 14.774 | 1.00 | 64.03 | C | |
| ATOM | 2240 | CB | ASN | A | 374 | 39.313 | −6.746 | 13.884 | 1.00 | 64.03 | C | |
| ATOM | 2243 | CG | ASN | A | 374 | 37.933 | −7.378 | 13.631 | 1.00 | 62.67 | C | |
| ATOM | 2244 | OD1 | ASN | A | 374 | 37.767 | −8.216 | 12.750 | 1.OQ | 60.24 | O | |
| ATOM | 2245 | ND2 | ASN | A | 374 | 36.951 | −6.968 | 14.420 | 1.00 | 60.66 | N | |
| ATOM | 2248 | C | ASN | A | 374 | 41.474 | −6.853 | 15.286 | 1.00 | 64.05 | C | |
| ATOM | 2249 | O | ASN | A | 374 | 41.811 | −5.759 | 14.794 | 1.00 | 63.76 | O | |
| ATOM | 2251 | N | HIS | A | 375 | 42.124 | −7.421 | 16.312 | 1.00 | 63.89 | N | |
| ATOM | 2252 | CA | HIS | A | 375 | 43.398 | −6.909 | 16.852 | 1.00 | 63.66 | C | |
| ATOM | 2254 | CB | HIS | A | 375 | 44.510 | −7.274 | 15.887 | 1.00 | 63.72 | C | |
| ATOM | 2257 | CG | HIS | A | 375 | 44.505 | −8.729 | 15.567 | 1.00 | 64.44 | C | |
| ATOM | 2258 | ND1 | HIS | A | 375 | 44.529 | −9.229 | 14.282 | 1.00 | 64.59 | N | |
| ATOM | 2260 | CE1 | HIS | A | 375 | 44.448 | −10.549 | 14.334 | 1.00 | 66.15 | C | |
| ATOM | 2262 | NE2 | HIS | A | 375 | 44.334 | −10.915 | 15.600 | 1.00 | 65.60 | N | |
| ATOM | 2264 | CD2 | HIS | A | 375 | 44.343 | −9.791 | 16.386 | 1.00 | 64.61 | C | |
| ATOM | 2266 | C | HIS | A | 375 | 43.379 | −5.439 | 17.225 | 1.006 | 3.18 | C | |
| ATOM | 2267 | O | HIS | A | 375 | 44.354 | −4.693 | 17.059 | 1.00 | 62.77 | O | |
| ATOM | 2269 | N | LEU | A | 376 | 42.223 | −5.055 | 17.763 | 1.00 | 62.88 | N | |
| ATOM | 2270 | CA | LEU | A | 376 | 42.065 | −3.800 | 18.464 | 1.00 | 62.41 | C | |
| ATOM | 2272 | CB | LEU | A | 376 | 40.586 | −3.527 | 18.815 | 1.00 | 62.22 | C | |
| ATOM | 2275 | CG | LED | A | 376 | 40.053 | −2.113 | 18.583 | 1.00 | 62.21 | C | |
| ATOM | 2277 | CD1 | LED | A | 376 | 38.750 | −1.959 | 19.332 | 1.00 | 62.60 | C | |
| ATOM | 2281 | CD2 | LED | A | 376 | 41.016 | −1.004 | 18.977 | 1.00 | 62.02 | C | |
| ATOM | 2285 | C | LEU | A | 376 | 42.903 | −3.998 | 19.733 | 1.00 | 61.90 | C | |
| ATOM | 2286 | O | LEU | A | 376 | 42.921 | −5.099 | 20.310 | 1.00 | 61.49 | O | |
| ATOM | 2288 | N | VAL | A | 377 | 43.652 | −2.966 | 20.109 | 1.00 | 61.32 | N | |
| ATOM | 2289 | CA | VAL | A | 377 | 44.405 | −2.953 | 21.367 | 1.00 | 60.57 | C | |
| ATOM | 2291 | CB | VAL | A | 377 | 45.921 | −3.264 | 21.182 | 1.00 | 60.17 | C | |
| ATOM | 2293 | CG1 | VAL | A | 377 | 46.658 | −3.204 | 22.514 | 1.00 | 60.72 | C | |
| ATOM | 2297 | CG2 | VAL | A | 377 | 46.122 | −4.628 | 20.584 | 1.00 | 59.80 | C | |
| ATOM | 2301 | C | VAL | A | 377 | 44.176 | −1.593 | 22.021 | 1.00 | 60.34 | C | |
| ATOM | 2302 | O | VAL | A | 377 | 44.138 | −0.546 | 21.334 | 1.00 | 59.94 | O | |
| ATOM | 2304 | N | LYS | A | 378 | 44.006 | −1.632 | 23.343 | 1.00 | 60.16 | N | |
| ATOM | 2305 | CA | LYS | A | 378 | 43.843 | −0.437 | 24.152 | 1.00 | 60.17 | C | |
| ATOM | 2307 | CB | LYS | A | 378 | 42.370 | −0.222 | 24.518 | 1.00 | 60.54 | C | |
| ATOM | 2310 | CG | LYS | A | 378 | 41.395 | −0.222 | 23.357 | 1.00 | 60.58 | C | |
| ATOM | 2313 | CD | LYS | A | 378 | 39.985 | 0.022 | 23.822 | 1.00 | 61.22 | C | |
| ATOM | 2316 | CE | LYS | A | 378 | 38.995 | −0.089 | 22.654 | 1.00 | 62.94 | C | |
| ATOM | 2319 | NZ | LYS | A | 378 | 37.887 | 0.911 | 22.722 | 1.00 | 63.18 | N | |
| ATOM | 2323 | C | LYS | A | 378 | 44.672 | −0.516 | 25.424 | 1.00 | 59.64 | C | |
| ATOM | 2324 | O | LYS | A | 378 | 44.861 | −1.575 | 26.006 | 1.00 | 58.88 | O | |
| ATOM | 2326 | N | VAL | A | 379 | 45.112 | 0.650 | 25.865 | 1.00 | 60.09 | N | |
| ATOM | 2327 | CA | VAL | A | 379 | 45.884 | 0.794 | 27.076 | 1.00 | 60.50 | C | |
| ATOM | 2329 | CB | VAL | A | 379 | 46.772 | 2.065 | 27.070 | 1.00 | 60.61 | C | |
| ATOM | 2331 | CG1 | VAL | A | 379 | 47.792 | 1.991 | 28.187 | 1.00 | 61.44 | C | |
| ATOM | 2335 | CG2 | VAL | A | 379 | 47.499 | 2.262 | 25.734 | 1.00 | 60.54 | C | |
| ATOM | 2339 | C | VAL | A | 379 | 44.854 | 0.863 | 28.227 | 1.00 | 60.94 | C | |
| ATOM | 2340 | O | VAL | A | 379 | 43.787 | 1.477 | 28.091 | 1.00 | 60.32 | O | |
| ATOM | 2342 | N | ALA | A | 380 | 45.213 | 0.226 | 29.346 | 1.00 | 61.40 | N | |
| ATOM | 2343 | CA | ALA | A | 380 | 44.341 | −0.021 | 30.495 | 1.00 | 61.23 | C | |
| ATOM | 2345 | CB | ALA | A | 380 | 43.790 | −1.413 | 30.392 | 1.00 | 60.79 | C | |
| ATOM | 2349 | C | ALA | A | 380 | 45.121 | 0.155 | 31.813 | 1.00 | 61.71 | C | |
| ATOM | 2350 | O | ALA | A | 380 | 46.338 | 0.471 | 31.800 | 1.00 | 62.08 | O | |
| ATOM | 2352 | N | ASP | A | 381 | 44.420 | −0.068 | 32.930 | 1.00 | 61.96 | N | |
| ATOM | 2353 | CA | ASP | A | 381 | 44.981 | −0.051 | 34.294 | 1.00 | 61.39 | C | |
| ATOM | 2355 | CB | ASP | A | 381 | 45.960 | −1.225 | 34.529 | 1.00 | 61.73 | C | |
| ATOM | 2358 | CG | ASP | A | 381 | 46.388 | −1.377 | 36.031 | 1.00 | 64.32 | C | |
| ATOM | 2359 | OD1 | ASP | A | 381 | 45.914 | −0.607 | 36.914 | 1.00 | 69.30 | O | |
| ATOM | 2360 | OD2 | ASP | A | 381 | 47.223 | −2.231 | 36.435 | 1.00 | 70.09 | O | |
| ATOM | 2361 | C | ASP | A | 381 | 45.566 | 1.312 | 34.669 | 1.00 | 60.20 | C | |
| ATOM | 2362 | O | ASP | A | 381 | 46.757 | 1.485 | 34.749 | 1.00 | 60.44 | O | |
| ATOM | 2364 | N | PHE | A | 382 | 44.695 | 2.265 | 34.944 | 1.00 | 59.40 | N | |
| ATOM | 2365 | CA | PHE | A | 382 | 45.109 | 3.669 | 35.118 | 1.00 | 59.19 | C | |
| ATOM | 2367 | CB | PHE | A | 382 | 44.075 | 4.579 | 34.440 | 1.00 | 59.11 | C | |
| ATOM | 2370 | CG | PHE | A | 382 | 43.970 | 4.330 | 32.955 | 1.00 | 59.61 | C | |
| ATOM | 2371 | CD1 | PHE | A | 382 | 44.955 | 4.831 | 32.093 | 1.00 | 58.60 | C | |
| ATOM | 2373 | CE1 | PEE | A | 382 | 44.894 | 4.573 | 30.745 | 1.00 | 59.35 | C | |
| ATOM | 2375 | CZ | PHE | A | 382 | 43.826 | 3.785 | 30.219 | 1.00 | 59.59 | C | |
| ATOM | 2377 | CE2 | PHE | A | 382 | 42.843 | 3.269 | 31.071 | 1.00 | 57.52 | C | |
| ATOM | 2379 | CD2 | PHE | A | 382 | 42.928 | 3.530 | 32.426 | 1.00 | 58.91 | C | |
| ATOM | 2381 | C | PHE | A | 382 | 45.409 | 4.108 | 36.571 | 1.00 | 59.00 | C | |
| ATOM | 2382 | O | PHE | A | 382 | 45.592 | 5.307 | 36.827 | 1.00 | 57.78 | O | |
| ATOM | 2384 | N | GLY | A | 383 | 45.488 | 3.124 | 37.489 | 1.00 | 58.98 | N | |
| ATOM | 2385 | CA | GLY | A | 383 | 45.755 | 3.333 | 38.920 | 1.00 | 58.78 | C | |
| ATOM | 2388 | C | GLY | A | 383 | 46.906 | 4.253 | 39.279 | 1.00 | 58.72 | C | |
| ATOM | 2389 | O | GLY | A | 383 | 46.735 | 5.106 | 40.129 | 1.00 | 58.62 | O | |
| ATOM | 2391 | N | LEU | A | 384 | 48.054 | 4.086 | 38.613 | 1.00 | 58.79 | N | |
| ATOM | 2392 | CA | LEU | A | 384 | 49.228 | 4.901 | 38.843 | 1.00 | 59.18 | C | |
| ATOM | 2394 | CB | LEU | A | 384 | 50.506 | 4.080 | 38.670 | 1.00 | 59.34 | C | |
| ATOM | 2397 | CG | LEU | A | 384 | 50.791 | 2.720 | 39.306 | 1.00 | 59.79 | C | |
| ATOM | 2399 | CD1 | LEU | A | 384 | 52.253 | 2.387 | 39.059 | 1.00 | 59.46 | C | |
| ATOM | 2403 | CD2 | LEU | A | 384 | 50.500 | 2.670 | 40.780 | 1.00 | 61.54 | C | |
| ATOM | 2407 | C | LEU | A | 384 | 49.325 | 6.100 | 37.892 | 1.00 | 60.19 | C | |
| ATOM | 2408 | O | LEU | A | 384 | 50.370 | 6.775 | 37.852 | 1.00 | 60.78 | O | |
| ATOM | 2410 | N | SER | A | 385 | 48.297 | 6.358 | 37.087 | 1.00 | 60.74 | N | |
| ATOM | 2411 | CA | SER | A | 385 | 48.351 | 7.453 | 36.128 | 1.00 | 60.72 | C | |
| ATOM | 2413 | CB | SER | A | 385 | 47.319 | 7.294 | 35.052 | 1.00 | 60.64 | C | |
| ATOM | 2416 | OG | SER | A | 385 | 47.427 | 5.988 | 34.565 | 1.00 | 64.31 | O | |
| ATOM | 2418 | C | SER | A | 385 | 48.058 | 8.707 | 36.861 | 1.00 | 60.72 | C | |
| ATOM | 2419 | O | SER | A | 385 | 47.093 | 8.744 | 37.622 | 1.00 | 61.12 | O | |
| ATOM | 2421 | N | ARG | A | 386 | 48.882 | 9.722 | 36.613 | 1.00 | 60.79 | N | |
| ATOM | 2422 | CA | ARG | A | 386 | 48.759 | 11.049 | 37.197 | 1.00 | 60.48 | C | |
| ATOM | 2424 | CB | ARG | A | 386 | 49.986 | 11.336 | 38.053 | 1.00 | 60.63 | C | |
| ATOM | 2427 | CG | ARG | A | 386 | 50.223 | 10.342 | 39.194 | 1.00 | 61.37 | C | |
| ATOM | 2430 | CD | ARG | A | 386 | 49.496 | 10.639 | 40.518 | 1.00 | 62.33 | C | |
| ATOM | 2433 | NE | ARG | A | 386 | 49.484 | 9.438 | 41.368 | 1.00 | 63.78 | N | |
| ATOM | 2435 | CZ | ARG | A | 386 | 48.658 | 8.373 | 41.237 | 1.00 | 63.16 | C | |
| ATOM | 2436 | NH1 | ARG | A | 386 | 47.751 | 8.309 | 40.269 | 1.00 | 63.46 | N | |
| ATOM | 2439 | NH2 | ARG | A | 386 | 48.767 | 7.351 | 42.078 | 1.00 | 62.04 | N | |
| ATOM | 2442 | C | ARG | A | 386 | 48.683 | 12.052 | 36.067 | 1.00 | 60.06 | C | |
| ATOM | 2443 | O | ARG | A | 386 | 49.185 | 11.777 | 35.006 | 1.00 | 59.42 | O | |
| ATOM | 2445 | N | LEU | A | 387 | 47.998 | 13.173 | 36.277 | 1.00 | 60.69 | N | |
| ATOM | 2446 | CA | LEU | A | 387 | 48.208 | 14.364 | 35.458 | 1.00 | 61.26 | C | |
| ATOM | 2448 | CB | LEU | A | 387 | 46.953 | 15.219 | 35.264 | 1.00 | 61.15 | C | |
| ATOM | 2451 | CG | LEU | A | 387 | 47.238 | 16.599 | 34.593 | 1.00 | 61.19 | C | |
| ATOM | 2453 | CD1 | LEU | A | 387 46.324 | 16.852 | 33.421 | 1.00 | 62.20 | C | | |
| ATOM | 2457 | CD2 | LEU | A | 387 47.169 | 17.767 | 35.585 | 1.00 | 61.56 | C | | |
| ATOM | 2461 | C | LEU | A | 387 | 49.236 | 15.160 | 36.213 | 1.00 | 61.64 | C | |
| ATOM | 2462 | O | LEU | A | 387 | 48.963 | 15.583 | 37.322 | 1.00 | 62.25 | O | |
| ATOM | 2464 | N | MET | A | 388 | 50.392 | 15.384 | 35.609 | 1.00 | 62.04 | N | |
| ATOM | 2465 | CA | MET | A | 388 | 51.443 | 16.138 | 36.268 | 1.00 | 62.74 | C | |
| ATOM | 2467 | CB | MET | A | 388 | 52.799 | 15.786 | 35.713 | 1.00 | 62.94 | C | |
| ATOM | 2470 | CG | MET | A | 388 | 53.157 | 14.351 | 35.830 | 1.00 | 63.04 | C | |
| ATOM | 2473 | SD | MET | A | 388 | 54.862 | 14.300 | 35.451 | 1.00 | 64.63 | S | |
| ATOM | 2474 | CE | MET | A | 388 | 54.904 | 14.808 | 33.677 | 1.00 | 64.63 | C | |
| ATOM | 2478 | C | MET | A | 388 | 51.260 | 17.634 | 36.084 | 1.00 | 62.32 | C | |
| ATOM | 2479 | O | MET | A | 388 | 50.929 | 18.093 | 35.003 | 1.00 | 61.54 | O | |
| ATOM | 2481 | N | THR | A | 389 | 51.460 | 18.362 | 37.171 | 1.00 | 62.55 | N | |
| ATOM | 2482 | CA | THR | A | 389 | 51.901 | 19.742 | 37.116 | 1.00 | 63.55 | C | |
| ATOM | 2484 | CB | THR | A | 389 | 51.387 | 20.489 | 38.326 | 1.00 | 64.05 | C | |
| ATOM | 2486 | OG1 | THR | A | 389 | 49.969 | 20.259 | 38.454 | 1.00 | 66.31 | O | |
| ATOM | 2488 | CG2 | THR | A | 389 | 51.533 | 21.992 | 38.128 | 1.00 | 64.34 | C | |
| ATOM | 2492 | C | THR | A | 389 | 53.428 | 19.676 | 37.125 | 1.00 | 63.41 | C | |
| ATOM | 2493 | O | THR | A | 389 | 54.011 | 18.898 | 37.927 | 1.00 | 64.15 | O | |
| ATOM | 2495 | N | GLY | A | 390 | 54.067 | 20.434 | 36.224 | 1.00 | 62.43 | N | |
| ATOM | 2496 | CA | GLY | A | 390 | 55.496 | 20.236 | 35.906 | 1.00 | 62.03 | C | |
| ATOM | 2499 | C | GLY | A | 390 | 55.840 | 18.940 | 35.165 | 1.00 | 61.54 | C | |
| ATOM | 2500 | O | GLY | A | 390 | 54.976 | 18.119 | 34.922 | 1.00 | 61.59 | O | |
| ATOM | 2502 | N | ASP | A | 391 | 57.123 | 18.731 | 34.864 | 1.00 | 61.75 | N | |
| ATOM | 2503 | CA | ASP | A | 391 | 57.595 | 17.617 | 33.992 | 1.00 | 61.90 | C | |
| ATOM | 2505 | CB | ASP | A | 391 | 58.784 | 18.103 | 33.142 | 1.00 | 62.78 | C | |
| ATOM | 2508 | CG | ASP | A | 391 | 58.349 | 18.819 | 31.894 | 1.00 | 64.21 | C | |
| ATOM | 2509 | OD1 | ASP | A | 391 | 57.596 | 18.206 | 31.094 | 1.00 | 65.43 | O | |
| ATOM | 2510 | OD2 | ASP | A | 391 | 58.732 | 19.982 | 31.631 | 1.00 | 68.16 | O | |
| ATOM | 2511 | C | ASP | A | 391 | 58.000 | 16.325 | 34.677 | 1.00 | 61.28 | C | |
| ATOM | 2512 | O | ASP | A | 391 | 58.539 | 15.424 | 34.015 | 1.00 | 60.82 | O | |
| ATOM | 2514 | N | THR | A | 392 | 57.665 | 16.200 | 35.964 | 1.00 | 60.74 | N | |
| ATOM | 2515 | CA | THR | A | 392 | 58.343 | 15.292 | 36.890 | 1.00 | 60.12 | C | |
| ATOM | 2517 | CB | THR | A | 392 | 59.593 | 16.038 | 37.477 | 1.00 | 60.00 | C | |
| ATOM | 2519 | OG1 | THR | A | 392 | 60.513 | 16.348 | 36.429 | 1.00 | 61.67 | O | |
| ATOM | 2521 | CG2 | THR | A | 392 | 60.402 | 15.174 | 38.416 | 1.00 | 59.60 | C | |
| ATOM | 2525 | C | THR | A | 392 | 57.429 | 14.914 | 38.047 | 1.00 | 59.38 | C | |
| ATOM | 2526 | O | THR | A | 392 | 57.183 | 15.755 | 38.933 | 1.00 | 60.19 | O | |
| ATOM | 2528 | N | TYR | A | 393 | 56.942 | 13.679 | 38.053 | 1.00 | 58.12 | N | |
| ATOM | 2529 | CA | TYR | A | 393 | 56.362 | 13.070 | 39.252 | 1.00 | 57.57 | C | |
| ATOM | 2531 | CB | TYR | A | 393 | 55.152 | 12.158 | 38.897 | 1.00 | 58.36 | C | |
| ATOM | 2534 | CG | TYR | A | 393 | 54.597 | 11.353 | 40.060 | 1.00 | 58.49 | C | |
| ATOM | 2535 | CD1 | TYR | A | 393 | 55.248 | 10.199 | 40.485 | 1.00 | 59.65 | C | |
| ATOM | 2537 | CE1 | TYR | A | 393 | 54.801 | 9.438 | 41.560 | 1.00 | 60.00 | C | |
| ATOM | 2539 | CZ | TYR | A | 393 | 53.671 | 9.805 | 42.255 | 1.00 | 59.96 | C | |
| ATOM | 2540 | OH | TYR | A | 393 | 53.353 | 8.931 | 43.277 | 1.00 | 61.50 | O | |
| ATOM | 2542 | CE2 | TYR | A | 393 | 52.936 | 10.952 | 41.864 | 1.00 | 58.92 | C | |
| ATOM | 2544 | CD2 | TYR | A | 393 | 53.427 | 11.737 | 40.742 | 1.00 | 60.07 | C | |
| ATOM | 2546 | C | TYR | A | 393 | 57.491 | 12.290 | 39.938 | 1.00 | 56.90 | C | |
| ATOM | 2547 | O | TYR | A | 393 | 58.133 | 11.490 | 39.295 | 1.00 | 56.34 | O | |
| ATOM | 2549 | N | THR | A | 394 | 57.674 | 12.502 | 41.244 | 1.00 | 56.49 | N | |
| ATOM | 2550 | CA | THR | A | 394 | 58.640 | 11.778 | 42.088 | 1.00 | 56.57 | C | |
| ATOM | 2552 | CB | THR | A | 394 | 59.473 | 12.799 | 42.937 | 1.00 | 55.92 | C | |
| ATOM | 2554 | OG1 | THR | A | 394 | 60.456 | 13.427 | 42.112 | 1.00 | 54.95 | O | |
| ATOM | 2556 | CG2 | THR | A | 394 | 60.316 | 12.146 | 44.051 | 1.00 | 55.31 | C | |
| ATOM | 2560 | C | THR | A | 394 | 57.925 | 10.738 | 42.984 | 1.00 | 56.54 | C | |
| ATOM | 2561 | O | THR | A | 394 | 57.113 | 11.092 | 43.797 | 1.00 | 55.96 | O | |
| ATOM | 2563 | N | ALA | A | 395 | 58.251 | 9.453 | 42.848 | 1.00 | 57.70 | N | |
| ATOM | 2564 | CA | ALA | A | 395 | 57.638 | 8.413 | 43.686 | 1.00 | 58.38 | C | |
| ATOM | 2566 | CB | ALA | A | 395 | 57.925 | 7.036 | 43.156 | 1.00 | 58.60 | C | |
| ATOM | 2570 | C | ALA | A | 395 | 58.112 | 8.508 | 45.122 | 1.00 | 60.18 | C | |
| ATOM | 2571 | O | ALA | A | 395 | 59.196 | 9.025 | 45.398 | 1.00 | 60.48 | O | |
| ATOM | 2573 | N | HIS | A | 396 | 57.300 | 8.006 | 46.041 | 1.00 | 62.13 | N | |
| ATOM | 2574 | CA | HIS | A | 396 | 57.701 | 7.974 | 47.432 | 1.00 | 63.98 | C | |
| ATOM | 2576 | CB | HIS | A | 396 | 56.511 | 7.723 | 48.374 | 1.00 | 64.98 | C | |
| ATOM | 2579 | CG | HIS | A | 396 | 56.808 | 8.051 | 49.807 | 1.00 | 67.58 | C | |
| ATOM | 2580 | ND1 | HIS | A | 396 | 57.407 | 9.239 | 50.191 | 1.00 | 68.28 | N | |
| ATOM | 2582 | CE1 | HIS | A | 396 | 57.581 | 9.230 | 51.506 | 1.00 | 70.33 | C | |
| ATOM | 2584 | NE2 | HIS | A | 396 | 57.101 | 8.090 | 51.990 | 1.00 | 70.58 | N | |
| ATOM | 2586 | CD2 | HIS | A | 396 | 56.617 | 7.332 | 50.947 | 1.00 | 70.45 | C | |
| ATOM | 2588 | C | HIS | A | 396 | 58.787 | 6.903 | 47.539 | 1.00 | 64.38 | C | |
| ATOM | 2589 | O | HIS | A | 396 | 58.811 | 5.953 | 46.730 | 1.00 | 65.07 | O | |
| ATOM | 2591 | N | ALA | A | 397 | 59.718 | 7.099 | 48.478 | 1.00 | 64.69 | N | |
| ATOM | 2592 | CA | ALA | A | 397 | 60.857 | 6.194 | 48.650 | 1.00 | 64.75 | C | |
| ATOM | 2594 | CB | ALA | A | 397 | 61.914 | 6.774 | 49.523 | 1.00 | 65.08 | C | |
| ATOM | 2598 | C | ALA | A | 397 | 60.335 | 4.955 | 49.265 | 1.00 | 65.23 | C | |
| ATOM | 2599 | O | ALA | A | 397 | 59.335 | 4.978 | 50.000 | 1.00 | 65.93 | O | |
| ATOM | 2601 | N | GLY | A | 398 | 60.983 | 3.854 | 48.929 | 1.00 | 65.61 | N | |
| ATOM | 2602 | CA | GLY | A | 398 | 60.417 | 2.557 | 49.211 | 1.00 | 66.37 | C | |
| ATOM | 2605 | C | GLY | A | 398 | 59.133 | 2.249 | 48.450 | 1.00 | 66.61 | C | |
| ATOM | 2606 | O | GLY | A | 398 | 58.406 | 1.332 | 48.842 | 1.00 | 67.56 | O | |
| ATOM | 2608 | N | ALA | A | 399 | 58.854 | 2.972 | 47.356 | 1.00 | 66.29 | N | |
| ATOM | 2609 | CA | ALA | A | 399 | 57.878 | 2.494 | 46.373 | 1.00 | 66.12 | C | |
| ATOM | 2611 | CB | ALA | A | 399 | 57.481 | 3.606 | 45.381 | 1.00 | 65.80 | C | |
| ATOM | 2615 | C | ALA | A | 399 | 58.483 | 1.256 | 45.673 | 1.00 | 65.50 | C | |
| ATOM | 2616 | O | ALA | A | 399 | 59.698 | 1.065 | 45.651 | 1.00 | 65.20 | O | |
| ATOM | 2618 | N | LYS | A | 400 | 57.625 | 0.377 | 45.196 | 1.00 | 65.33 | N | |
| ATOM | 2619 | CA | LYS | A | 400 | 58.072 | −0.848 | 44.518 | 1.00 | 65.47 | C | |
| ATOM | 2621 | CB | LYS | A | 400 | 57.428 | −2.082 | 45.132 | 1.00 | 65.39 | C | |
| ATOM | 2624 | CG | LYS | A | 400 | 57.992 | −2.423 | 46.515 | 1.00 | 66.42 | C | |
| ATOM | 2630 | C | LYS | A | 400 | 57.705 | −0.688 | 43.044 | 1.00 | 65.09 | C | |
| ATOM | 2631 | O | LYS | A | 400 | 56.561 | −0.336 | 42.732 | 1.00 | 66.11 | O | |
| ATOM | 2633 | N | PHE | A | 401 | 58.699 | −0.878 | 42.168 | 1.00 | 63.74 | N | |
| ATOM | 2634 | CA | PHE | A | 401 | 58.560 | −0.753 | 40.731 | 1.00 | 62.36 | C | |
| ATOM | 2636 | CB | PHE | A | 401 | 59.710 | 0.054 | 40.184 | 1.00 | 62.44 | C | |
| ATOM | 2639 | CG | PHE | A | 401 | 59.883 | 1.398 | 40.808 | 1.00 | 63.52 | C | |
| ATOM | 2640 | CD1 | PHE | A | 401 | 58.787 | 2.176 | 41.202 | 1.00 | 63.93 | C | |
| ATOM | 2642 | CE1 | PHE | A | 401 | 58.994 | 3.437 | 41.767 | 1.00 | 64.70 | C | |
| ATOM | 2644 | CZ | PHE | A | 401 | 60.298 | 3.955 | 41.891 | 1.00 | 63.13 | C | |
| ATOM | 2646 | CE2 | PHE | A | 401 | 61.367 | 3.224 | 41.464 | 1.00 | 62.81 | C | |
| ATOM | 2648 | CD2 | PHE | A | 401 | 61.167 | 1.944 | 40.923 | 1.00 | 63.99 | C | |
| ATOM | 2650 | C | PHE | A | 401 | 58.709 | −2.106 | 40.072 | 1.00 | 61.65 | C | |
| ATOM | 2651 | O | PHE | A | 401 | 59.452 | −2.968 | 40.621 | 1.00 | 62.75 | O | |
| ATOM | 2653 | N | PRO | A | 402 | 58.121 | −2.291 | 38.875 | 1.00 | 59.69 | N | |
| ATOM | 2654 | CA | PRO | A | 402 | 58.587 | −3.376 | 37.972 | 1.00 | 57.88 | C | |
| ATOM | 2656 | CB | PRO | A | 402 | 57.463 | −3.482 | 36.939 | 1.00 | 58.48 | C | |
| ATOM | 2659 | CG | PRO | A | 402 | 56.849 | −2.058 | 36.889 | 1.00 | 58.79 | C | |
| ATOM | 2662 | CD | PRO | A | 402 | 57.010 | −1.520 | 38.275 | 1.00 | 59.09 | C | |
| ATOM | 2665 | C | PRO | A | 402 | 59.942 | −2.978 | 37.351 | 1.00 | 56.59 | C | |
| ATOM | 2666 | O | PRO | A | 402 | 59.985 | −2.318 | 36.344 | 1.00 | 55.60 | O | |
| ATOM | 2667 | N | ILE | A | 403 | 61.029 | −3.376 | 37.999 | 1.00 | 55.79 | N | |
| ATOM | 2668 | CA | ILE | A | 403 | 62.403 | −2.962 | 37.650 | 1.00 | 55.33 | C | |
| ATOM | 2670 | CB | ILE | A | 403 | 63.463 | −3.887 | 38.367 | 1.00 | 55.76 | C | |
| ATOM | 2672 | CG1 | ILE | A | 403 | 63.390 | −3.790 | 39.909 | 1.00 | 56.03 | C | |
| ATOM | 2675 | CD1 | ILE | A | 403 | 63.737 | −2.428 | 40.498 | 1.00 | 55.85 | C | |
| ATOM | 2679 | CG2 | ILE | A | 403 | 64.899 | −3.579 | 37.888 | 1.00 | 56.38 | C | |
| ATOM | 2683 | C | ILE | A | 403 | 62.668 | −2.993 | 36.145 | 1.00 | 54.38 | C | |
| ATOM | 2684 | O | ILE | A | 403 | 62.990 | −1.961 | 35.560 | 1.00 | 53.73 | O | |
| ATOM | 2686 | N | LYS | A | 404 | 62.455 | −4.173 | 35.543 | 1.00 | 53.63 | N | |
| ATOM | 2687 | CA | LYS | A | 404 | 62.794 | −4.474 | 34.134 | 1.00 | 52.99 | C | |
| ATOM | 2689 | CB | LYS | A | 404 | 62.629 | −5.964 | 33.851 | 1.00 | 52.71 | C | |
| ATOM | 2692 | CG | LYS | A | 404 | 63.702 | −6.801 | 34.540 | 1.00 | 54.23 | C | |
| ATOM | 2695 | CD | LYS | A | 404 | 63.345 | −8.306 | 34.508 | 1.00 | 56.00 | C | |
| ATOM | 2698 | CE | LYS | A | 404 | 64.416 | −9.240 | 35.160 | 1.00 | 57.15 | C | |
| ATOM | 2701 | NZ | LYS | A | 404 | 65.179 | −10.091 | 34.197 | 1.00 | 57.27 | N | |
| ATOM | 2705 | C | LYS | A | 404 | 62.063 | −3.650 | 33.061 | 1.00 | 51.78 | C | |
| ATOM | 2706 | O | LYS | A | 404 | 62.398 | −3.748 | 31.892 | 1.00 | 51.95 | O | |
| ATOM | 2708 | N | TRP | A | 405 | 61.064 | −2.877 | 33.463 | 1.00 | 50.55 | N | |
| ATOM | 2709 | CA | TRP | A | 405 | 60.387 | −1.874 | 32.646 | 1.00 | 49.89 | C | |
| ATOM | 2711 | CB | TRP | A | 405 | 58.868 | −1.993 | 32.841 | 1.00 | 48.43 C | | |
| ATOM | 2714 | CG | TRP | A | 405 | 58.321 | −3.029 | 32.076 | 1.00 | 46.94 | C | |
| ATOM | 2715 | CD1 | TRP | A | 405 | 57.691 | −2.891 | 30.900 | 1.00 | 46.16 | C | |
| ATOM | 2717 | NE1 | TRP | A | 405 | 57.303 | −4.114 | 30.431 | 1.00 | 46.67 | N | |
| ATOM | 2719 | CE2 | TRP | A | 405 | 57.706 | −5.076 | 31.320 | 1.00 | 47.09 | C | |
| ATOM | 2720 | CD2 | TRP | A | 405 | 58.354 | −4.417 | 32.373 | 1.00 | 46.79 | C | |
| ATOM | 2721 | CE3 | TRP | A | 405 | 58.858 | −5.186 | 33.438 | 1.00 | 46.32 | C | |
| ATOM | 2723 | CZ3 | TRP | A | 405 | 58.701 | −6.510 | 33.414 | 1.00 | 44.90 | C | |
| ATOM | 2725 | CH2 | TRP | A | 405 | 58.063 | −7.135 | 32.354 | 1.00 | 45.72 | C | |
| ATOM | 2727 | CZ2 | TRP | A | 405 | 57.558 | −6.443 | 31.292 | 1.00 | 44.52 | C | |
| ATOM | 2729 | C | TRP | A | 405 | 60.716 | −0.479 | 33.046 | 1.00 | 49.55 | C | |
| ATOM | 2730 | O | TRP | A | 405 | 60.271 | 0.426 | 32.377 | 1.00 | 50.62 | O | |
| ATOM | 2732 | N | THR | A | 406 | 61.427 | −0.287 | 34.153 | 1.00 | 49.74 | N | |
| ATOM | 2733 | CA | THR | A | 406 | 61.627 | 1.037 | 34.747 | 1.00 | 49.85 | C | |
| ATOM | 2735 | CB | THR | A | 406 | 61.574 | 0.931 | 36.291 | 1.00 | 49.94 | C | |
| ATOM | 2737 | OG1 | THR | A | 406 | 60.329 | 0.335 | 36.706 | 1.00 | 48.47 | O | |
| ATOM | 2739 | CG2 | THR | A | 406 | 61.584 | 2.322 | 36.916 | 1.00 | 49.29 | C | |
| ATOM | 2743 | C | THR | A | 406 | 62.959 | 1.659 | 34.297 | 1.00 | 49.97 | C | |
| ATOM | 2744 | O | THR | A | 406 | 63.996 | 0.948 | 34.302 | 1.00 | 50.67 | O | |
| ATOM | 2746 | N | ALA | A | 407 | 62.919 | 2.960 | 33.983 | 1.00 | 49.61 | N | |
| ATOM | 2747 | CA | ALA | A | 407 | 64.068 | 3.733 | 33.448 | 1.00 | 49.64 | C | |
| ATOM | 2749 | CB | ALA | A | 407 | 63.591 | 5.048 | 32.847 | 1.00 | 49.55 | C | |
| ATOM | 2753 | C | ALA | A | 407 | 65.077 | 4.079 | 34.501 | 1.00 | 49.68 | C | |
| ATOM | 2754 | O | ALA | A | 407 | 64.675 | 4.253 | 35.628 | 1.00 | 50.21 | O | |
| ATOM | 2756 | N | PRO | A | 408 | 66.351 | 4.300 | 34.150 | 1.00 | 50.27 | N | |
| ATOM | 2757 | CA | PRO | A | 408 | 67.398 | 4.433 | 35.152 | 1.00 | 50.64 | C | |
| ATOM | 2759 | CB | PRO | A | 408 | 68.669 | 4.651 | 34.313 | 1.00 | 50.13 | C | |
| ATOM | 2762 | CG | PRO | A | 408 | 68.368 | 3.978 | 33.090 | 1.00 | 50.42 | C | |
| ATOM | 2765 | CD | PRO | A | 408 | 66.943 | 4.465 | 32.811 | 1.00 | 50.69 | C | |
| ATOM | 2768 | C | PRO | A | 408 | 67.207 | 5.546 | 36.169 | 1.00 | 51.16 | C | |
| ATOM | 2769 | O | PRO | A | 408 | 67.391 | 5.287 | 37.369 | 1.00 | 52.10 | O | |
| ATOM | 2770 | N | GLU | A | 409 | 66.848 | 6.750 | 35.724 | 1.00 | 51.37 | N | |
| ATOM | 2771 | CA | GLU | A | 409 | 66.573 | 7.860 | 36.663 | 1.00 | 51.89 | C | |
| ATOM | 2773 | CB | GLU | A | 409 | 66.367 | 9.212 | 35.970 | 1.00 | 52.46 | C | |
| ATOM | 2776 | CG | GLU | A | 409 | 65.099 | 9.363 | 35.098 | 1.00 | 54.26 | C | |
| ATOM | 2779 | CD | GLU | A | 409 | 65.120 | 8.562 | 33.809 | 1.00 | 52.61 | C | |
| ATOM | 2780 | OE1 | GLU | A | 409 | 66.165 | 7.944 | 33.500 | 1.00 | 54.56 | O | |
| ATOM | 2781 | OE2 | GLU | A | 409 | 64.099 | 8.550 | 33.123 | 1.00 | 50.79 | O | |
| ATOM | 2782 | C | GLU | A | 409 | 65.403 | 7.583 | 37.610 | 1.00 | 51.83 | C | |
| ATOM | 2783 | O | GLU | A | 409 | 65.378 | 8.126 | 38.728 | 1.00 | 52.36 | O | |
| ATOM | 2785 | N | SER | A | 410 | 64.453 | 6.741 | 37.178 | 1.00 | 51.16 | N | |
| ATOM | 2786 | CA | SER | A | 410 | 63.358 | 6.301 | 38.052 | 1.00 | 50.45 | C | |
| ATOM | 2788 | CB | SER | A | 410 | 62.209 | 5.688 | 37.254 | 1.00 | 50.22 | C | |
| ATOM | 2791 | OG | SER | A | 410 | 61.816 | 6.484 | 36.142 | 1.00 | 48.58 | O | |
| ATOM | 2793 | C | SER | A | 410 | 63.895 | 5.319 | 39.092 | 1.00 | 50.93 | C | |
| ATOM | 2794 | O | SER | A | 410 | 63.574 | 5.477 | 40.282 | 1.00 | 52.03 | O | |
| ATOM | 2796 | N | LEU | A | 411 | 64.735 | 4.345 | 38.691 | 1.00 | 50.30 | N | |
| ATOM | 2797 | CA | LEU | A | 411 | 65.378 | 3.423 | 39.685 | 1.00 | 50.08 | C | |
| ATOM | 2799 | CB | LEU | A | 411 | 66.111 | 2.254 | 39.022 | 1.00 | 50.19 | C | |
| ATOM | 2802 | CG | LEU | A | 411 | 65.339 | 1.288 | 38.090 | 1.00 | 51.68 | C | |
| ATOM | 2804 | CD1 | LEU | A | 411 | 66.281 | 0.454 | 37.196 | 1.00 | 51.31 | C | |
| ATOM | 2808 | CD2 | LEU | A | 411 | 64.487 | 0.375 | 38.863 | 1.00 | 52.17 | C | |
| ATOM | 2812 | C | LEU | A | 411 | 66.340 | 4.107 | 40.680 | 1.00 | 49.77 | C | |
| ATOM | 2813 | O | LEU | A | 411 | 66.444 | 3.651 | 41.796 | 1.00 | 50.26 | O | |
| ATOM | 2815 | N | ALA | A | 412 | 67.032 | 5.186 | 40.304 | 1.00 | 49.48 | N | |
| ATOM | 2816 | CA | ALA | A | 412 | 68.003 | 5.827 | 41.221 | 1.00 | 49.44 | C | |
| ATOM | 2818 | CB | ALA | A | 412 | 69.140 | 6.443 | 40.459 | 1.00 | 49.50 | C | |
| ATOM | 2822 | C | ALA | A | 412 | 67.342 | 6.873 | 42.088 | 1.00 | 49.35 | C | |
| ATOM | 2823 | O | ALA | A | 412 | 67.481 | 6.859 | 43.302 | 1.00 | 50.56 | O | |
| ATOM | 2825 | N | TYR | A | 413 | 66.589 | 7.762 | 41.459 | 1.00 | 49.00 | N | |
| ATOM | 2826 | CA | TYR | A | 413 | 66.062 | 8.963 | 42.102 | 1.00 | 48.50 | C | |
| ATOM | 2828 | CB | TYR | A | 413 | 66.554 | 10.140 | 41.287 | 1.00 | 47.26 | C | |
| ATOM | 2831 | CG | TYR | A | 413 | 68.047 | 10.061 | 41.059 | 1.00 | 47.26 | C | |
| ATOM | 2832 | CD1 | TYR | A | 413 | 68.926 | 10.069 | 42.138 | 1.00 | 46.98 | C | |
| ATOM | 2834 | CE1 | TYR | A | 413 | 70.315 | 10.033 | 41.940 | 1.00 | 46.70 | C | |
| ATOM | 2836 | CZ | TYR | A | 413 | 70.831 | 9.951 | 40.643 | 1.00 | 45.93 | C | |
| ATOM | 2837 | OH | TYR | A | 413 | 72.211 | 9.868 | 40.478 | 1.00 | 47.65 | O | |
| ATOM | 2839 | CE2 | TYR | A | 413 | 69.979 | 9.932 | 39.559 | 1.00 | 44.66 | C | |
| ATOM | 2841 | CD2 | TYR | A | 413 | 68.595 | 9.973 | 39.766 | 1.00 | 46.15 | C | |
| ATOM | 2843 | C | TYR | A | 413 | 64.539 | 9.007 | 42.228 | 1.00 | 48.63 | C | |
| ATOM | 2844 | O | TYR | A | 413 | 63.981 | 10.026 | 42.636 | 1.00 | 48.32 | O | |
| ATOM | 2846 | N | ASN | A | 414 | 63.859 | 7.916 | 41.865 | 1.00 | 49.52 | N | |
| ATOM | 2847 | CA | ASN | A | 414 | 62.378 | 7.903 | 41.738 | 1.00 | 50.04 | C | |
| ATOM | 2849 | CB | ASN | A | 414 | 61.698 | 7.874 | 43.109 | 1.00 | 49.50 | C | |
| ATOM | 2852 | CG | ASN | A | 414 | 62.299 | 6.888 | 44.014 | 1.00 | 48.81 | C | |
| ATOM | 2853 | OD1 | ASN | A | 414 | 62.072 | 5.691 | 43.893 | 1.00 | 50.48 | O | |
| ATOM | 2854 | ND2 | ASN | A | 414 | 63.103 | 7.373 | 44.942 | 1.00 | 51.56 | N | |
| ATOM | 2857 | C | ASN | A | 414 | 61.799 | 9.058 | 40.889 | 1.00 | 50.92 | C | |
| ATOM | 2858 | O | ASN | A | 414 | 60.690 | 9.510 | 41.141 | 1.00 | 50.65 | O | |
| ATOM | 2860 | N | LYS | A | 415 | 62.540 | 9.510 | 39.885 | 1.00 | 51.77 | N | |
| ATOM | 2861 | CA | LYS | A | 415 | 62.018 | 10.527 | 38.985 | 1.00 | 53.04 | C | |
| ATOM | 2863 | CB | LYS | A | 415 | 63.106 | 11.543 | 38.574 | 1.00 | 52.90 | C | |
| ATOM | 2866 | CG | LYS | A | 415 | 63.119 | 12.820 | 39.508 | 1.00 | 55.05 | C | |
| ATOM | 2869 | CD | LYS | A | 415 | 64.539 | 13.405 | 39.779 | 1.00 | 56.64 | C | |
| ATOM | 2872 | CE | LYS | A | 415 | 65.111 | 14.352 | 38.625 | 1.00 | 60.50 | C | |
| ATOM | 2875 | NZ | LYS | A | 415 | 66.036 | 13.655 | 37.638 | 1.00 | 63.58 | N | |
| ATOM | 2879 | C | LYS | A | 415 | 61.292 | 9.797 | 37.823 | 1.00 | 51.91 | C | |
| ATOM | 2880 | O | LYS | A | 415 | 61.906 | 9.052 | 37.065 | 1.00 | 51.90 | O | |
| ATOM | 2882 | N | PHE | A | 416 | 59.969 | 9.991 | 37.768 | 1.00 | 51.15 | N | |
| ATOM | 2883 | CA | PHE | A | 416 | 59.110 | 9.602 | 36.655 | 1.00 | 50.52 | C | |
| ATOM | 2885 | CB | PHE | A | 416 | 57.874 | 8.911 | 37.165 | 1.00 | 50.24 | C | |
| ATOM | 2888 | CG | PHE | A | 416 | 58.193 | 7.624 | 37.814 | 1.00 | 50.01 | C | |
| ATOM | 2889 | CD1 | PHE | A | 416 | 58.714 | 7.584 | 39.126 | 1.00 | 51.45 | C | |
| ATOM | 2891 | CE1 | PHE | A | 416 | 59.065 | 6.383 | 39.706 | 1.00 | 48.81 | C | |
| ATOM | 2893 | CZ | PHE | A | 416 | 58.908 | 5.228 | 38.983 | 1.00 | 48.80 | C | |
| ATOM | 2895 | CE2 | PHE | A | 416 | 58.432 | 5.268 | 37.704 | 1.00 | 49.22 | C | |
| ATOM | 2897 | CD2 | PHE | A | 416 | 58.095 | 6.463 | 37.115 | 1.00 | 48.43 | C | |
| ATOM | 2899 | C | PHE | A | 416 | 58.755 | 10.818 | 35.854 | 1.00 | 50.13 | C | |
| ATOM | 2900 | O | PHE | A | 416 | 58.834 | 11.914 | 36.380 | 1.00 | 51.13 | O | |
| ATOM | 2902 | N | SER | A | 417 | 58.439 | 10.599 | 34.568 | 1.00 | 49.55 | N | |
| ATOM | 2903 | CA | SER | A | 417 | 58.114 | 11.629 | 33.547 | 1.00 | 48.43 | C | |
| ATOM | 2905 | CB | SER | A | 417 | 59.363 | 12.349 | 33.188 | 1.00 | 47.84 | C | |
| ATOM | 2908 | OG | SER | A | 417 | 60.183 | 11.494 | 32.429 | 1.00 | 48.45 | O | |
| ATOM | 2910 | C | SER | A | 417 | 57.550 | 10.971 | 32.247 | 1.00 | 48.41 | C | |
| ATOM | 2911 | O | SER | A | 417 | 57.387 | 9.737 | 32.136 | 1.00 | 48.30 | O | |
| ATOM | 2913 | N | ILE | A | 418 | 57.257 | 11.772 | 31.242 | 1.00 | 48.13 | N | |
| ATOM | 2914 | CA | ILE | A | 418 | 56.745 | 11.189 | 29.996 | 1.00 | 48.24 | C | |
| ATOM | 2916 | CB | ILE | A | 418 | 56.293 | 12.302 | 29.062 | 1.00 | 48.42 | C | |
| ATOM | 2918 | CG1 | ILE | A | 418 | 55.074 | 13.010 | 29.686 | 1.00 | 50.54 | C | |
| ATOM | 2921 | CD1 | ILE | A | 418 | 55.021 | 14.519 | 29.436 | 1.00 | 53.60 | C | |
| ATOM | 2925 | CG2 | ILE | A | 418 | 55.901 | 11.743 | 27.689 | 1.00 | 48.57 | C | |
| ATOM | 2929 | C | ILE | A | 418 | 57.828 | 10.263 | 29.393 | 1.00 | 47.77 | C | |
| ATOM | 2930 | O | ILE | A | 418 | 57.569 | 9.106 | 29.032 | 1.00 | 47.92 | O | |
| ATOM | 2932 | N | LYS | A | 419 | 59.033 | 10.817 | 29.332 | 1.00 | 46.76 | N | |
| ATOM | 2933 | CA | LYS | A | 419 | 60.266 | 10.136 | 29.009 | 1.00 | 45.78 | C | |
| ATOM | 2935 | CB | LYS | A | 419 | 61.442 | 11.062 | 29.322 | 1.00 | 44.69 | C | |
| ATOM | 2938 | CG | LYS | A | 419 | 62.007 | 11.750 | 28.133 | 1.00 | 43.87 | C | |
| ATOM | 2941 | CD | LYS | A | 419 | 61.036 | 12.446 | 27.166 | 1.00 | 41.50 | C | |
| ATOM | 2944 | CE | LYS | A | 419 | 61.699 | 12.738 | 25.833 | 1.00 | 41.17 | C | |
| ATOM | 2947 | NZ | LYS | A | 419 | 60.662 | 13.163 | 24.853 | 1.00 | 42.34 | N | |
| ATOM | 2951 | C | LYS | A | 419 | 60.480 | 8.800 | 29.690 | 1.00 | 46.43 | C | |
| ATOM | 2952 | 0 | LYS | A | 419 | 60.977 | 7.865 | 29.045 | 1.00 | 45.91 | O | |
| ATOM | 2954 | N | SER | A | 420 | 60.122 | 8.700 | 30.968 | 1.00 | 46.65 | N | |
| ATOM | 2955 | CA | SER | A | 420 | 60.267 | 7.426 | 31.662 | 1.00 | 47.64 | C | |
| ATOM | 2957 | CB | SER | A | 420 | 60.252 | 7.623 | 33.183 | 1.00 | 47.41 | C | |
| ATOM | 2960 | OG | SER | A | 420 | 58.940 | 7.729 | 33.718 | 1.00 | 49.07 | O | |
| ATOM | 2962 | C | SER | A | 420 | 59.228 | 6.379 | 31.117 | 1.00 | 47.84 | C | |
| ATOM | 2963 | 0 | SER | A | 420 | 59.525 | 5.219 | 30.984 | 1.00 | 48.93 | O | |
| ATOM | 2965 | N | ASP | A | 421 | 58.041 | 6.812 | 30.748 | 1.00 | 48.57 | N | |
| ATOM | 2966 | CA | ASP | A | 421 | 57.112 | 5.984 | 29.952 | 1.00 | 48.99 | C | |
| ATOM | 2968 | CB | ASP | A | 421 | 55.701 | 6.662 | 29.830 | 1.00 | 49.45 | C | |
| ATOM | 2971 | CG | ASP | A | 421 | 54.994 | 6.807 | 31.162 | 1.00 | 51.17 | C | |
| ATOM | 2972 | OD1 | ASP | A | 421 | 55.559 | 6.471 | 32.249 | 1.00 | 51.48 | O | |
| ATOM | 2973 | OD2 | ASP | A | 421 | 53.837 | 7.259 | 31.211 | 1.00 | 55.51 | O | |
| ATOM | 2974 | C | ASP | A | 421 | 57.601 | 5.669 | 28.530 | 1.00 | 48.39 | C | |
| ATOM | 2975 | O | ASP | A | 421 | 57.162 | 4.668 | 27.952 | 1.00 | 48.16 | O | |
| ATOM | 2977 | N | VAL | A | 422 | 58.411 | 6.555 | 27.940 | 1.00 | 47.58 | N | |
| ATOM | 2978 | CA | VAL | A | 422 | 59.000 | 6.284 | 26.629 | 1.00 | 47.40 | C | |
| ATOM | 2980 | CB | VAL | A | 422 | 59.846 | 7.480 | 26.068 | 1.00 | 47.85 | C | |
| ATOM | 2982 | CG1 | VAL | A | 422 | 60.595 | 7.094 | 24.765 | 1.00 | 47.45 | C | |
| ATOM | 2986 | CG2 | VAL | A | 422 | 58.959 | 8.756 | 25.827 | 1.00 | 47.11 | C | |
| ATOM | 2990 | C | VAL | A | 422 | 59.865 | 5.040 | 26.783 | 1.00 | 47.21 | C | |
| ATOM | 2991 | O | VAL | A | 422 | 59.702 | 4.062 | 26.026 | 1.00 | 47.55 | O | |
| ATOM | 2993 | N | TRP | A | 423 | 60.729 | 5.061 | 27.802 | 1.00 | 46.57 | N | |
| ATOM | 2994 | CA | TRP | A | 423 | 61.530 | 3.895 | 28.192 | 1.00 | 45.98 | C | |
| ATOM | 2996 | CB | TRP | A | 423 | 62.315 | 4.194 | 29.492 | 1.00 | 46.04 | C | |
| ATOM | 2999 | CG | TRP | A | 423 | 63.273 | 3.090 | 29.897 | 1.00 | 47.36 | C | |
| ATOM | 3000 | CD1 | TRP | A | 423 | 62.929 | 1.889 | 30.432 | 1.00 | 47.55 | C | |
| ATOM | 3002 | NE1 | TRP | A | 423 | 64.052 | 1.153 | 30.668 | 1.00 | 48.29 | N | |
| ATOM | 3004 | CE2 | TRP | A | 423 | 65.153 | 1.846 | 30.261 | 1.00 | 46.14 | C | |
| ATOM | 3005 | CD2 | TRP | A | 423 | 64.704 | 3.073 | 29.767 | 1.00 | 46.11 | C | |
| ATOM | 3006 | CE3 | TRP | A | 423 | 65.652 | 3.985 | 29.303 | 1.00 | 47.79 | C | |
| ATOM | 3008 | CZ3 | TRP | A | 423 | 67.016 | 3.624 | 29.337 | 1.00 | 47.76 | C | |
| ATOM | 3010 | CH2 | TRP | A | 423 | 67.418 | 2.364 | 29.816 | 1.00 | 47.57 | C | |
| ATOM | 3012 | CZ2 | TRP | A | 423 | 66.506 | 1.470 | 30.276 | 1.00 | 46.69 | C | |
| ATOM | 3014 | C | TRP | A | 423 | 60.692 | 2.604 | 28.297 | 1.00 | 45.21 | C | |
| ATOM | 3015 | O | TRP | A | 423 | 60.979 | 1.595 | 27.654 | 1.00 | 43.59 | O | |
| ATOM | 3017 | N | ALA | A | 424 | 59.642 | 2.709 | 29.101 | 1.00 | 45.77 | N | |
| ATOM | 3018 | CA | ALA | A | 424 | 58.621 | 1.654 | 29.322 | 1.00 | 46.14 | C | |
| ATOM | 3020 | CB | ALA | A | 424 | 57.497 | 2.209 | 30.182 | 1.00 | 46.41 | C | |
| ATOM | 3024 | C | ALA | A | 424 | 58.011 | 1.084 | 28.035 | 1.00 | 46.30 | C | |
| ATOM | 3025 | O | ALA | A | 424 | 57.834 | −0.136 | 27.876 | 1.00 | 43.97 | O | |
| ATOM | 3027 | N | PHE | A | 425 | 57.683 | 2.015 | 27.139 | 1.00 | 47.45 | N | |
| ATOM | 3028 | CA | PHE | A | 425 | 57.122 | 1.689 | 25.828 | 1.00 | 48.30 | C | |
| ATOM | 3030 | CB | PHE | A | 425 | 56.640 | 2.956 | 25.073 | 1.00 | 48.93 | C | |
| ATOM | 3033 | CG | PHE | A | 425 | 56.116 | 2.656 | 23.715 | 1.00 | 50.33 | C | |
| ATOM | 3034 | CD1 | PHE | A | 425 | 54.797 | 2.214 | 23.550 | 1.00 | 50.66 | C | |
| ATOM | 3036 | CE1 | PHE | A | 425 | 54.303 | 1.848 | 22.258 | 1.00 | 50.15 | C | |
| ATOM | 3038 | CZ | PHE | A | 425 | 55.157 | 1.935 | 21.131 | 1.00 | 51.09 | C | |
| ATOM | 3040 | CE2 | PHE | A | 425 | 56.506 | 2.345 | 21.293 | 1.00 | 50.74 | C | |
| ATOM | 3042 | CD2 | PHE | A | 425 | 56.974 | 2.705 | 22.591 | 1.00 | 51.55 | C | |
| ATOM | 3044 | C | PHE | A | 425 | 58.144 | 0.879 | 25.018 | 1.00 | 47.58 | C | |
| ATOM | 3045 | O | PHE | A | 425 | 57.793 | −0.164 | 24.448 | 1.00 | 47.13 | O | |
| ATOM | 3047 | N | GLY | A | 426 | 59.395 | 1.344 | 25.007 | 1.00 | 47.25 | N | |
| ATOM | 3048 | CA | GLY | A | 426 | 60.474 | 0.628 | 24.308 | 1.00 | 47.10 | C | |
| ATOM | 3051 | C | GLY | A | 426 | 60.579 | −0.851 | 24.701 | 1.00 | 46.27 | C | |
| ATOM | 3052 | O | GLY | A | 426 | 60.798 | −1.720 | 23.857 | 1.00 | 45.66 | O | |
| ATOM | 3054 | N | VAL | A | 427 | 60.405 | −1.110 | 25.993 | 1.00 | 45.34 | N | |
| ATOM | 3055 | CA | VAL | A | 427 | 60.470 | −2.460 | 26.545 | 1.00 | 45.21 | C | |
| ATOM | 3057 | CB | VAL | A | 427 | 60.648 | −2.402 | 28.125 | 1.00 | 45.04 | C | |
| ATOM | 3059 | CG1 | VAL | A | 427 | 60.593 | −3.814 | 28.824 | 1.00 | 45.31 | C | |
| ATOM | 3063 | CG2 | VAL | A | 427 | 61.955 | −1.732 | 28.445 | 1.00 | 44.03 | C | |
| ATOM | 3067 | C | VAL | A | 427 | 59.263 | −3.311 | 26.097 | 1.00 | 44.99 | C | |
| ATOM | 3068 | O | VAL | A | 427 | 59.430 | −4.486 | 25.729 | 1.00 | 45.67 | O | |
| ATOM | 3070 | N | LEU | A | 428 | 58.073 | −2.719 | 26.171 | 1.00 | 44.78 | N | |
| ATOM | 3071 | CA | LEU | A | 428 | 56.855 | −3.254 | 25.548 | 1.00 | 44.69 | C | |
| ATOM | 3073 | CB | LEU | A | 428 | 55.670 | −2.289 | 25.785 | 1.00 | 45.09 | C | |
| ATOM | 3076 | CG | LEU | A | 428 | 54.375 | −2.388 | 24.956 | 1.00 | 45.86 | C | |
| ATOM | 3078 | CD1 | LEU | A | 428 | 53.662 | −3.677 | 25.258 | 1.00 | 48.58 | C | |
| ATOM | 3082 | CD2 | LEU | A | 428 | 53.466 | −1.224 | 25.226 | 1.00 | 45.75 | C | |
| ATOM | 3086 | C | LEU | A | 428 | 57.012 | −3.612 | 24.035 | 1.00 | 44.14 | C | |
| ATOM | 3087 | O | LEU | A | 428 | 56.494 | −4.647 | 23.593 | 1.00 | 42.31 | O | |
| ATOM | 3089 | N | LEU | A | 429 | 57.711 | −2.777 | 23.257 | 1.00 | 43.88 | N | |
| ATOM | 3090 | CA | LEU | A | 429 | 58.003 | −3.154 | 21.871 | 1.00 | 44.20 | C | |
| ATOM | 3092 | CB | LEU | A | 429 | 58.809 | −2.114 | 21.096 | 1.00 | 43.64 | C | |
| ATOM | 3095 | CG | LEU | A | 429 | 58.079 | −0.832 | 20.655 | 1.00 | 45.00 | C | |
| ATOM | 3097 | CD1 | LEU | A | 429 | 59.048 | 0.175 | 19.948 | 1.00 | 43.66 | C | |
| ATOM | 3101 | CD2 | LEU | A | 429 | 56.826 | −1.102 | 19.746 | 1.00 | 44.75 | C | |
| ATOM | 3105 | C | LEU | A | 429 | 58.757 | −4.463 | 21.889 | 1.00 | 44.59 | C | |
| ATOM | 3106 | O | LEU | A | 429 | 58.407 | −5.379 | 21.137 | 1.00 | 45.60 | O | |
| ATOM | 3108 | N | TRP | A | 430 | 59.743 | −4.567 | 22.783 | 1.00 | 44.42 | N | |
| ATOM | 3109 | CA | TRP | A | 430 | 60.551 | −5.770 | 22.916 | 1.00 | 44.32 | C | |
| ATOM | 3111 | CB | TRP | A | 430 | 61.713 | −5.537 | 23.917 | 1.00 | 43.34 | C | |
| ATOM | 3114 | CG | TRP | A | 430 | 62.747 | −6.578 | 23.849 | 1.00 | 42.11 | C | |
| ATOM | 3115 | CD1 | TRP | A | 430 | 63.896 | −6.518 | 23.155 | 1.00 | 41.67 | C | |
| ATOM | 3117 | NE1 | TRP | A | 430 | 64.586 | −7.698 | 23.290 | 1.00 | 41.49 | N | |
| ATOM | 3119 | CE2 | TRP | A | 430 | 63.873 | −8.547 | 24.085 | 1.00 | 39.79 | C | |
| ATOM | 3120 | CD2 | TRP | A | 430 | 62.704 | −7.872 | 24.459 | 1.00 | 40.36 | C | |
| ATOM | 3121 | CE3 | TRP | A | 430 | 61.782 | −8.541 | 25.275 | 1.00 | 40.59 | C | |
| ATOM | 3123 | CZ3 | TRP | A | 430 | 62.093 | −9.840 | 25.723 | 1.00 | 41.66 | C | |
| ATOM | 3125 | CH2 | TRP | A | 430 | 63.266 | −10.467 | 25.335 | 1.00 | 41.06 | C | |
| ATOM | 3127 | CZ2 | TRP | A | 430 | 64.169 | −9.837 | 24.514 | 1.00 | 40.31 | C | |
| ATOM | 3129 | C | TRP | A | 430 | 59.660 | −6.980 | 23.286 | 1.00 | 44.66 | C | |
| ATOM | 3130 | O | TRP | A | 430 | 59.860 | −8.097 | 22.781 | 1.00 | 42.95 | O | |
| ATOM | 3132 | N | GLU | A | 431 | 58.653 | −6.737 | 24.133 | 1.00 | 45.66 | N | |
| ATOM | 3133 | CA | GLU | A | 431 | 57.661 | −7.788 | 24.447 | 1.00 | 46.28 | C | |
| ATOM | 3135 | CB | GLU | A | 431 | 56.694 | −7.365 | 25.549 | 1.00 | 45.74 | C | |
| ATOM | 3138 | CG | GLU | A | 431 | 57.320 | −6.917 | 26.868 | 1.00 | 46.68 | C | |
| ATOM | 3141 | CD | GLU | A | 431 | 56.252 | −6.568 | 27.912 | 1.00 | 48.88 | C | |
| ATOM | 3142 | OE1 | GLU | A | 431 | 55.767 | −7.497 | 28.625 | 1.00 | 50.05 | O | |
| ATOM | 3143 | OE2 | GLU | A | 431 | 55.862 | −5.351 | 27.986 | 1.00 | 53.25 | O | |
| ATOM | 3144 | C | GLU | A | 431 | 56.868 | −8.210 | 23.222 | 1.00 | 46.59 | C | |
| ATOM | 3145 | O | GLU | A | 431 | 56.596 | −9.390 | 23.051 | 1.00 | 48.89 | O | |
| ATOM | 3147 | N | ILE | A | 432 | 56.501 | −7.256 | 22.377 | 1.00 | 46.79 | N | |
| ATOM | 3148 | CA | ILE | A | 432 | 55.615 | −7.497 | 21.235 | 1.00 | 47.22 | C | |
| ATOM | 3150 | CB | ILE | A | 432 | 55.051 | −6.126 | 20.679 | 1.00 | 47.19 | C | |
| ATOM | 3152 | CG1 | ILE | A | 432 | 53.999 | −5.578 | 21.647 | 1.00 | 47.56 | C | |
| ATOM | 3155 | CD1 | ILE | A | 432 | 53.571 | −4.143 | 21.409 | 1.00 | 47.69 | C | |
| ATOM | 3159 | CG2 | ILE | A | 432 | 54.388 | −6.299 | 19.338 | 1.00 | 47.88 | C | |
| ATOM | 3163 | C | ILE | A | 432 | 56.387 | −8.263 | 20.189 | 1.00 | 47.72 | C | |
| ATOM | 3164 | O | ILE | A | 432 | 55.940 | −9.291 | 19.720 | 1.00 | 47.80 | O | |
| ATOM | 3166 | N | ALA | A | 433 | 57.560 | −7.743 | 19.855 | 1.00 | 49.10 | N | |
| ATOM | 3167 | CA | ALA | A | 433 | 58.541 | −8.380 | 18.943 | 1.00 | 50.08 | C | |
| ATOM | 3169 | CB | ALA | A | 433 | 59.800 | −7.538 | 18.876 | 1.00 | 50.50 | C | |
| ATOM | 3173 | C | ALA | A | 433 | 58.954 | −9.799 | 19.243 | 1.00 | 50.16 | C | |
| ATOM | 3174 | O | ALA | A | 433 | 59.299 | −10.498 | 18.327 | 1.00 | 51.09 | O | |
| ATOM | 3176 | N | THR | A | 434 | 58.933 | −10.210 | 20.507 | 1.00 | 51.09 | N | |
| ATOM | 3177 | CA | THR | A | 434 | 59.255 | −11.600 | 20.930 | 1.00 | 51.38 | C | |
| ATOM | 3179 | CB | THR | A | 434 | 60.117 | −11.546 | 22.217 | 1.00 | 51.73 | C | |
| ATOM | 3181 | OG1 | THR | A | 434 | 59.392 | −10.824 | 23.222 | 1.00 | 53.77 | O | |
| ATOM | 3183 | CG2 | THR | A | 434 | 61.451 | −10.758 | 22.003 | 1.00 | 51.31 | C | |
| ATOM | 3187 | C | THR | A | 434 | 58.050 | −12.461 | 21.247 | 1.00 | 51.17 | C | |
| ATOM | 3188 | O | THR | A | 434 | 58.212 | −13.584 | 21.753 | 1.00 | 51.41 | O | |
| ATOM | 3190 | N | TYR | A | 435 | 56.848 | −11.948 | 21.010 | 1.00 | 51.51 | N | |
| ATOM | 3191 | CA | TYR | A | 435 | 55.588 | −12.628 | 21.455 | 1.00 | 51.75 | C | |
| ATOM | 3193 | CB | TYR | A | 435 | 55.271 | −13.902 | 20.633 | 1.00 | 50.84 | C | |
| ATOM | 3196 | CG | TYR | A | 435 | 55.007 | −13.606 | 19.196 | 1.00 | 49.66 | C | |
| ATOM | 3197 | CD1 | TYR | A | 435 | 56.054 | −13.454 | 18.293 | 1.00 | 49.18 | C | |
| ATOM | 3199 | CE1 | TYR | A | 435 | 55.817 | −13.172 | 16.960 | 1.00 | 48.20 | C | |
| ATOM | 3201 | CZ | TYR | A | 435 | 54.533 | −13.045 | 16.530 | 1.00 | 48.92 | C | |
| ATOM | 3202 | OH | TYR | A | 435 | 54.287 | −12.748 | 15.253 | 1.00 | 50.40 | O | |
| ATOM | 3204 | CE2 | TYR | A | 435 | 53.474 | −13.177 | 17.389 | 1.00 | 49.60 | C | |
| ATOM | 3206 | CD2 | TYR | A | 435 | 53.719 | −13.448 | 18.731 | 1.00 | 49.74 | C | |
| ATOM | 3208 | C | TYR | A | 435 | 55.595 | −12.949 | 22.942 | 1.00 | 51.79 | C | |
| ATOM | 3209 | O | TYR | A | 435 | 55.373 | −14.099 | 23.349 | 1.00 | 50.77 | O | |
| ATOM | 3211 | N | GLY | A | 436 | 55.907 | −11.918 | 23.718 | 1.00 | 52.32 | N | |
| ATOM | 3212 | CA | GLY | A | 436 | 55.778 | −11.971 | 25.161 | 1.00 | 53.64 | C | |
| ATOM | 3215 | C | GLY | A | 436 | 56.827 | −12.726 | 25.962 | 1.00 | 54.48 | C | |
| ATOM | 3216 | O | GLY | A | 436 | 56.488 | −13.339 | 26.971 | 1.00 | 54.70 | O | |
| ATOM | 3218 | N | MET | A | 437 | 58.092 | −12.674 | 25.541 | 1.00 | 55.41 | N | |
| ATOM | 3219 | CA | MET | A | 437 | 59.222 | −13.059 | 26.426 | 1.00 | 56.42 | C | |
| ATOM | 3221 | CB | MET | A | 437 | 60.539 | −13.157 | 25.647 | 1.00 | 56.49 | C | |
| ATOM | 3224 | CG | MET | A | 437 | 60.745 | −14.482 | 24.876 | 1.00 | 59.17 | C | |
| ATOM | 3227 | SD | MET | A | 437 | 62.386 | −14.383 | 23.998 | 1.00 | 62.41 | S | |
| ATOM | 3228 | CE | MET | A | 437 | 63.728 | −15.039 | 25.513 | 1.00 | 64.10 | C | |
| ATOM | 3232 | C | MET | A | 437 | 59.373 | −12.019 | 27.543 | 1.00 | 54.60 | C | |
| ATOM | 3233 | O | MET | A | 437 | 59.088 | −10.826 | 27.324 | 1.00 | 54.63 | O | |
| ATOM | 3235 | N | SER | A | 438 | 59.796 | −12.469 | 28.728 | 1.00 | 53.13 | N | |
| ATOM | 3236 | CA | SER | A | 438 | 60.126 | −11.562 | 29.839 | 1.00 | 52.40 | C | |
| ATOM | 3238 | CB | SER | A | 438 | 60.311 | −12.296 | 31.139 | 1.00 | 52.37 | C | |
| ATOM | 3241 | OG | SER | A | 438 | 59.146 | −13.010 | 31.450 | 1.00 | 56.00 | O | |
| ATOM | 3243 | C | SER | A | 438 | 61.439 | −10.849 | 29.566 | 1.00 | 51.38 | C | |
| ATOM | 3244 | O | SER | A | 438 | 62.426 | −11.516 | 29.142 | 1.00 | 51.57 | O | |
| ATOM | 3246 | N | PRO | A | 439 | 61.489 | −9.535 | 29.806 | 1.00 | 49.65 | N | |
| ATOM | 3247 | CA | PRO | A | 439 | 62.725 | −8.768 | 29.564 | 1.00 | 48.82 | C | |
| ATOM | 3249 | CB | PRO | A | 439 | 62.327 | −7.312 | 29.846 | 1.00 | 48.91 | C | |
| ATOM | 3252 | CG | PRO | A | 439 | 60.891 | −7.297 | 30.014 | 1.00 | 49.15 | C | |
| ATOM | 3255 | CD | PRO | A | 439 | 60.384 | −8.665 | 30.211 | 1.00 | 49.33 | C | |
| ATOM | 3258 | C | PRO | A | 439 | 63.966 | −9.199 | 30.380 | 1.00 | 48.32 | C | |
| ATOM | 3259 | O | PRO | A | 439 | 63.831 | −9.837 | 31.423 | 1.00 | 47.61 | O | |
| ATOM | 3260 | N | TYR | A | 440 | 65.147 | −8.864 | 29.833 | 1.00 | 47.63 | N | |
| ATOM | 3261 | CA | TYR | A | 440 | 66.470 | −9.221 | 30.382 | 1.00 | 46.71 | C | |
| ATOM | 3263 | CB | TYR | A | 440 | 66.863 | −8.267 | 31.536 | 1.00 | 46.57 | C | |
| ATOM | 3266 | CG | TYR | A | 440 | 66.886 | −6.885 | 31.098 | 1.00 | 44.68 | C | |
| ATOM | 3267 | CD1 | TYR | A | 440 | 67.974 | −6.396 | 30.403 | 1.00 | 46.62 | C | |
| ATOM | 3269 | CE1 | TYR | A | 440 | 68.027 | −5.047 | 29.980 | 1.00 | 47.17 | C | |
| ATOM | 3271 | CZ | TYR | A | 440 | 66.919 | −4.220 | 30.203 | 1.00 | 45.95 | C | |
| ATOM | 3272 | OH | TYR | A | 440 | 66.935 | −2.965 | 29.729 | 1.00 | 47.49 | O | |
| ATOM | 3274 | CE2 | TYR | A | 440 | 65.812 | −4.686 | 30.864 | 1.00 | 45.22 | C | |
| ATOM | 3276 | CD2 | TYR | A | 440 | 65.797 | −6.049 | 31.310 | 1.00 | 46.32 | C | |
| ATOM | 3278 | C | TYR | A | 440 | 66.493 | −10.643 | 30.840 | 1.00 | 44.68 | C | |
| ATOM | 3279 | O | TYR | A | 440 | 66.759 | −10.886 | 31.995 | 1.00 | 42.62 | O | |
| ATOM | 3281 | N | PRO | A | 441 | 66.203 | −11.568 | 29.927 | 1.00 | 44.78 | N | |
| ATOM | 3282 | CA | PRO | A | 441 | 65.956 | −12.991 | 30.288 | 1.00 | 45.49 | C | |
| ATOM | 3284 | CB | PRO | A | 441 | 65.764 | −13.703 | 28.939 | 1.00 | 44.71 | C | |
| ATOM | 3287 | CG | PRO | A | 441 | 65.906 | −12.720 | 27.900 | 1.00 | 43.56 | C | |
| ATOM | 3290 | CD | PRO | A | 441 | 66.163 | −11.371 | 28.474 | 1.00 | 44.55 | C | |
| ATOM | 3293 | C | PRO | A | 441 | 67.132 | −13.588 | 31.003 | 1.00 | 46.24 | C | |
| ATOM | 3294 | O | PRO | A | 441 | 68.257 | −13.486 | 30.507 | 1.00 | 46.98 | O | |
| ATOM | 3295 | N | GLY | A | 442 | 66.898 | −14.083 | 32.201 | 1.00 | 47.65 | N | |
| ATOM | 3296 | CA | GLY | A | 442 | 67.916 | −14.854 | 32.924 | 1.00 | 49.12 | C | |
| ATOM | 3299 | C | GLY | A | 442 | 68.727 | −14.030 | 33.894 | 1.00 | 50.64 | C | |
| ATOM | 3300 | O | GLY | A | 442 | 69.505 | −14.603 | 34.649 | 1.00 | 51.14 | O | |
| ATOM | 3302 | N | ILE | A | 443 | 68.511 | −12.711 | 33.901 | 1.00 | 52.0 | N | |
| ATOM | 3303 | CA | ILE | A | 443 | 69.234 | −11.765 | 34.724 | 1.00 | 53.8 | C | |
| ATOM | 3305 | CB | ILE | A | 443 | 69.487 | −10.475 | 33.908 | 1.00 | 53.6 | C | |
| ATOM | 3307 | CG1 | ILE | A | 443 | 70.501 | −10.793 | 32.808 | 1.00 | 54.1 | C | |
| ATOM | 3310 | CD1 | ILE | A | 443 | 70.717 | −9.696 | 31.838 | 1.00 | 54.3 | C | |
| ATOM | 3314 | CG2 | ILE | A | 443 | 69.945 | −9.274 | 34.808 | 1.00 | 53.6 | C | |
| ATOM | 3318 | C | ILE | A | 443 | 68.414 | −11.447 | 35.974 | 1.00 | 54.9 | C | |
| ATOM | 3319 | O | ILE | A | 443 | 67.226 | −11.138 | 35.862 | 1.00 | 55.9 | O | |
| ATOM | 3321 | N | ASP | A | 444 | 69.055 | −11.498 | 37.148 | 1.00 | 56.0 | N | |
| ATOM | 3322 | CA | ASP | A | 444 | 68.446 | −11.029 | 38.413 | 1.00 | 56.7 | C | |
| ATOM | 3324 | CB | ASP | A | 444 | 69.280 | −11.414 | 39.667 | 1.00 | 57.8 | C | |
| ATOM | 3327 | CG | ASP | A | 444 | 70.776 | −10.879 | 39.617 | 1.00 | 62.5 | C | |
| ATOM | 3328 | OD1 | ASP | A | 444 | 71.319 | −10.611 | 38.473 | 1.00 | 66.8 | O | |
| ATOM | 3329 | OD2 | ASP | A | 444 | 71.489 | −10.731 | 40.670 | 1.00 | 65.1 | O | |
| ATOM | 3330 | C | ASP | A | 444 | 68.233 | −9.512 | 38.407 | 1.00 | 56.44 | C | |
| ATOM | 3331 | O | ASP | A | 444 | 69.109 | −8.750 | 37.955 | 1.00 | 55.83 | O | |
| ATOM | 3333 | N | LEU | A | 445 | 67.073 | −9.101 | 38.942 | 1.00 | 55.87 | N | |
| ATOM | 3334 | CA | LEU | A | 445 | 66.749 | −7.690 | 39.215 | 1.00 | 55.54 | C | |
| ATOM | 3336 | CB | LEU | A | 445 | 65.589 | −7.584 | 40.190 | 1.00 | 55.50 | C | |
| ATOM | 3339 | CG | LEU | A | 445 | 64.288 | −8.311 | 39.969 | 1.00 | 56.17 | C | |
| ATOM | 3341 | CD1 | LEU | A | 445 | 63.266 | −7.790 | 41.002 | 1.00 | 55.87 | C | |
| ATOM | 3345 | CD2 | LEU | A | 445 | 63.821 | −8.150 | 38.519 | 1.00 | 56.83 | C | |
| ATOM | 3349 | C | LEU | A | 445 | 67.872 | −6.862 | 39.831 | 1.00 | 55.12 | C | |
| ATOM | 3350 | O | LEU | A | 445 | 68.017 | −5.702 | 39.516 | 1.00 | 55.02 | O | |
| ATOM | 3352 | N | SER | A | 446 | 68.617 | −7.475 | 40.741 | 1.00 | 54.92 | N | |
| ATOM | 3353 | CA | SER | A | 446 | 69.707 | −6.818 | 41.461 | 1.00 | 54.79 | C | |
| ATOM | 3355 | CB | SER | A | 446 | 70.156 | −7.709 | 42.625 | 1.00 | 54.76 | C | |
| ATOM | 3358 | OG | SER | A | 446 | 70.105 | −9.061 | 42.203 | 1.00 | 56.56 | O | |
| ATOM | 3360 | C | SER | A | 446 | 70.933 | −6.446 | 40.619 | 1.00 | 54.57 | C | |
| ATOM | 3361 | O | SER | A | 446 | 71.752 | −5.686 | 41.109 | 1.00 | 54.74 | O | |
| ATOM | 3363 | N | GLN | A | 447 | 71.082 | −6.979 | 39.396 | 1.00 | 54.00 | N | |
| ATOM | 3364 | CA | GLN | A | 447 | 72.150 | −6.504 | 38.467 | 1.00 | 53.08 | C | |
| ATOM | 3366 | CB | GLN | A | 447 | 72.994 | −7.687 | 37.996 | 1.00 | 52.87 | C | |
| ATOM | 3369 | CG | GLN | A | 447 | 73.694 | −8.352 | 39.212 | 1.00 | 52.82 | C | |
| ATOM | 3372 | CD | GLN | A | 447 | 74.741 | −9.404 | 38.852 | 1.00 | 52.72 | C | |
| ATOM | 3373 | OE1 | GLN | A | 447 | 75.873 | −9.059 | 38.407 | 1.00 | 49.36 | O | |
| ATOM | 3374 | NE2 | GLN | A | 447 | 74.397 | −10.690 | 39.091 | 1.00 | 49.26 | N | |
| ATOM | 3377 | C | GLN | A | 447 | 71.708 | −5.553 | 37.322 | 1.00 | 52.34 | C | |
| ATOM | 3378 | O | GLN | A | 447 | 72.546 | −4.864 | 36.739 | 1.00 | 52.85 | O | |
| ATOM | 3380 | N | VAL | A | 448 | 70.409 | −5.422 | 37.095 | 1.00 | 51.23 | N | |
| ATOM | 3381 | CA | VAL | A | 448 | 69.886 | −4.681 | 35.952 | 1.00 | 50.51 | C | |
| ATOM | 3383 | CB | VAL | A | 448 | 68.348 | −4.711 | 35.905 | 1.00 | 49.82 | C | |
| ATOM | 3385 | CG1 | VAL | A | 448 | 67.820 | −3.814 | 34.810 | 1.00 | 50.00 | C | |
| ATOM | 3389 | CG2 | VAL | A | 448 | 67.862 | −6.132 | 35.644 | 1.00 | 49.15 | C | |
| ATOM | 3393 | C | VAL | A | 448 | 70.428 | −3.254 | 35.888 | 1.00 | 49.77 | C | |
| ATOM | 3394 | O | VAL | A | 448 | 70.989 | −2.881 | 34.885 | 1.00 | 49.91 | O | |
| ATOM | 3396 | N | TYR | A | 449 | 70.315 | −2.481 | 36.956 | 1.00 | 49.39 | N | |
| ATOM | 3397 | CA | TYR | A | 449 | 70.724 | −1.048 | 36.922 | 1.00 | 49.55 | C | |
| ATOM | 3399 | CB | TYR | A | 449 | 70.418 | −0.261 | 38.229 | 1.00 | 47.59 | C | |
| ATOM | 3402 | CG | TYR | A | 449 | 70.910 | 1.191 | 38.208 | 1.00 | 47.42 | C | |
| ATOM | 3403 | CD1 | TYR | A | 449 | 72.234 | 1.511 | 38.549 | 1.00 | 44.15 | C | |
| ATOM | 3405 | CE1 | TYR | A | 449 | 72.701 | 2.803 | 38.502 | 1.00 | 43.51 | C | |
| ATOM | 3407 | CZ | TYR | A | 449 | 71.847 | 3.862 | 38.124 | 1.00 | 44.96 | C | |
| ATOM | 3408 | OH | TYR | A | 449 | 72.346 | 5.196 | 38.116 | 1.00 | 44.92 | O | |
| ATOM | 3410 | CE2 | TYR | A | 449 | 70.527 | 3.579 | 37.794 | 1.00 | 43.62 | C | |
| ATOM | 3412 | CD2 | TYR | A | 449 | 70.063 | 2.247 | 37.842 | 1.00 | 45.18 | C | |
| ATOM | 3414 | C | TYR | A | 449 | 72.190 | −0.953 | 36.566 | 1.00 | 50.41 | C | |
| ATOM | 3415 | O | TYR | A | 449 | 72.508 | −0.295 | 35.603 | 1.00 | 51.50 | O | |
| ATOM | 3417 | N | GLU | A | 450 | 73.068 | −1.611 | 37.316 | 1.00 | 51.84 | N | |
| ATOM | 3418 | CA | GLU | A | 450 | 74.511 | −1.628 | 36.982 | 1.00 | 53.36 | C | |
| ATOM | 3420 | CB | GLU | A | 450 | 75.264 | −2.664 | 37.793 | 1.00 | 54.22 | C | |
| ATOM | 3423 | CG | GLU | A | 450 | 75.283 | −2.370 | 39.276 | 1.00 | 61.11 | C | |
| ATOM | 3426 | CD | GLU | A | 450 | 76.679 | −2.074 | 39.841 | 1.00 | 69.67 | C | |
| ATOM | 3427 | OE1 | GLU | A | 450 | 77.608 | −2.926 | 39.566 | 1.00 | 75.16 | O | |
| ATOM | 3428 | OE2 | GLU | A | 450 | 76.829 | −1.029 | 40.578 | 1.00 | 71.51 | O | |
| ATOM | 3429 | C | GLU | A | 450 | 74.794 | −1.935 | 35.507 | 1.00 | 52.66 | C | |
| ATOM | 3430 | 0 | GLU | A | 450 | 75.678 | −1.297 | 34.888 | 1.00 | 53.17 | O | |
| ATOM | 3432 | N | LEU | A | 451 | 74.062 | −2.903 | 34.953 | 1.00 | 51.14 | N | |
| ATOM | 3433 | CA | LEU | A | 451 | 74.248 | −3.250 | 33.544 | 1.00 | 50.23 | C | |
| ATOM | 3435 | CB | LEU | A | 451 | 73.541 | −4.551 | 33.218 | 1.00 | 49.97 | C | |
| ATOM | 3438 | CG | LEU | A | 451 | 74.094 | −5.783 | 33.893 | 1.00 | 48.69 | C | |
| ATOM | 3440 | CD1 | LEU | A | 451 | 73.174 | −6.955 | 33.683 | 1.00 | 48.35 | C | |
| ATOM | 3444 | CD2 | LEU | A | 451 | 75.438 | −6.104 | 33.319 | 1.00 | 51.44 | C | |
| ATOM | 3448 | C | LEU | A | 451 | 73.810 | −2.131 | 32.595 | 1.00 | 49.31 | C | |
| ATOM | 3449 | O | LEU | A | 451 | 74.529 | −1.800 | 31.645 | 1.00 | 48.75 | O | |
| ATOM | 3451 | N | LEU | A | 452 | 72.675 | −1.522 | 32.884 | 1.00 | 48.98 | N | |
| ATOM | 3452 | CA | LEU | A | 452 | 72.252 | −0.304 | 32.141 | 1.00 | 49.73 | C | |
| ATOM | 3454 | CB | LEU | A | 452 | 70.850 | 0.186 | 32.558 | 1.00 | 49.09 | C | |
| ATOM | 3457 | CG | LEU | A | 452 | 69.688 | −0.834 | 32.498 | 1.00 | 48.08 | C | |
| ATOM | 3459 | CD1 | LEU | A | 452 | 68.464 | −0.367 | 33.250 | 1.00 | 47.95 | C | |
| ATOM | 3463 | CD2 | LEU | A | 452 | 69.302 | −1.204 | 31.099 | 1.00 | 47.26 | C | |
| ATOM | 3467 | C | LEU | A | 452 | 73.309 | 0.828 | 32.245 | 1.00 | 50.02 | C | |
| ATOM | 3468 | O | LEU | A | 452 | 73.639 | 1.443 | 31.241 | 1.00 | 50.47 | O | |
| ATOM | 3470 | N | GLU | A | 453 | 73.894 | 1.029 | 33.430 | 1.00 | 50.20 | N | |
| ATOM | 3471 | CA | GLU | A | 453 | 74.995 | 2.000 | 33.617 | 1.00 | 50.60 | C | |
| ATOM | 3473 | CB | GLU | A | 453 | 75.392 | 2.093 | 35.087 | 1.00 | 50.82 | C | |
| ATOM | 3476 | CG | GLU | A | 453 | 76.242 | 3.328 | 35.399 | 1.00 | 51.95 | C | |
| ATOM | 3479 | CD | GLU | A | 453 | 76.359 | 3.540 | 36.892 | 1.00 | 54.05 | C | |
| ATOM | 3480 | OE1 | GLU | A | 453 | 76.746 | 2.547 | 37.586 | 1.00 | 61.43 | O | |
| ATOM | 3481 | OE2 | GLU | A | 453 | 76.055 | 4.668 | 37.378 | 1.00 | 58.31 | O | |
| ATOM | 3482 | C | GLU | A | 453 | 76.261 | 1.711 | 32.809 | 1.00 | 49.47 | C | |
| ATOM | 3483 | O | GLU | A | 453 | 76.847 | 2.621 | 32.233 | 1.00 | 49.30 | O | |
| ATOM | 3485 | N | LYS | A | 454 | 76.680 | 0.446 | 32.809 | 1.00 | 48.70 | N | |
| ATOM | 3486 | CA | LYS | A | 454 | 77.765 | −0.022 | 31.950 | 1.00 | 48.14 | C | |
| ATOM | 3488 | CB | LYS | A | 454 | 78.368 | −1.313 | 32.488 | 1.00 | 48.10 | C | |
| ATOM | 3491 | CG | LYS | A | 454 | 78.985 | −1.153 | 33.840 | 1.00 | 47.78 | C | |
| ATOM | 3494 | CD | LYS | A | 454 | 80.230 | −1.960 | 33.986 | 1.00 | 48.32 | C | |
| ATOM | 3497 | CE | LYS | A | 454 | 80.950 | −1.591 | 35.252 | 1.00 | 50.37 | C | |
| ATOM | 3500 | NZ | LYS | A | 454 | 80.330 | −2.232 | 36.415 | 1.00 | 51.50 | N | |
| ATOM | 3504 | C | LYS | A | 454 | 77.338 | −0.200 | 30.489 | 1.00 | 48.14 | C | |
| ATOM | 3505 | O | LYS | A | 454 | 78.129 | −0.660 | 29.674 | 1.00 | 47.25 | O | |
| ATOM | 3507 | N | ASP | A | 455 | 76.091 | 0.149 | 30.159 | 1.00 | 48.43 | N | |
| ATOM | 3508 | CA | ASP | A | 455 | 75.672 | 0.427 | 28.772 | 1.00 | 48.30 | C | |
| ATOM | 3510 | CB | ASP | A | 455 | 76.795 | 1.242 | 28.023 | 1.00 | 48.40 | C | |
| ATOM | 3513 | CG | ASP | A | 455 | 76.350 | 1.855 | 26.716 | 1.00 | 49.21 | C | |
| ATOM | 3514 | OD1 | ASP | A | 455 | 75.111 | 1.971 | 26.447 | 1.00 | 51.06 | O | |
| ATOM | 3515 | OD2 | ASP | A | 455 | 77.225 | 2.227 | 25.882 | 1.00 | 50.21 | O | |
| ATOM | 3516 | C | ASP | A | 455 | 75.241 | −0.873 | 28.061 | 1.00 | 47.27 | C | |
| ATOM | 3517 | O | ASP | A | 455 | 75.527 | −1.064 | 26.886 | 1.00 | 46.60 | O | |
| ATOM | 3519 | N | TYR | A | 456 | 74.573 | −1.759 | 28.810 | 1.00 | 46.29 | N | |
| ATOM | 3520 | CA | TYR | A | 456 | 74.049 | −3.012 | 28.282 | 1.00 | 45.50 | C | |
| ATOM | 3522 | CB | TYR | A | 456 | 74.186 | −4.178 | 29.272 | 1.00 | 45.82 | C | |
| ATOM | 3525 | CG | TYR | A | 456 | 73.548 | −5.448 | 28.732 | 1.00 | 45.17 | C | |
| ATOM | 3526 | CD1 | TYR | A | 456 | 74.217 | −6.231 | 27.820 | 1.00 | 46.67 | C | |
| ATOM | 3528 | CE1 | TYR | A | 456 | 73.620 | −7.382 | 27.300 | 1.00 | 48.52 | C | |
| ATOM | 3530 | CZ | TYR | A | 456 | 72.330 | −7.699 | 27.653 | 1.00 | 45.78 | C | |
| ATOM | 3531 | OH | TYR | A | 456 | 71.788 | −8.817 | 27.146 | 1.00 | 46.00 | O | |
| ATOM | 3533 | CE2 | TYR | A | 456 | 71.643 | −6.914 | 28.521 | 1.00 | 46.03 | C | |
| ATOM | 3535 | CD2 | TYR | A | 456 | 72.246 | −5.804 | 29.058 | 1.00 | 44.81 | C | |
| ATOM | 3537 | C | TYR | A | 456 | 72.573 | −2.822 | 27.999 | 1.00 | 45.07 | C | |
| ATOM | 3538 | O | TYR | A | 456 | 71.860 | −2.247 | 28.822 | 1.00 | 44.69 | O | |
| ATOM | 3540 | N | ARG | A | 457 | 72.120 | −3.335 | 26.852 | 1.00 | 44.67 | N | |
| ATOM | 3541 | CA | ARG | A | 457 | 70.721 | −3.301 | 26.445 | 1.00 | 44.17 | C | |
| ATOM | 3543 | CB | ARG | A | 457 | 70.439 | −2.169 | 25.473 | 1.00 | 43.75 | C | |
| ATOM | 3546 | CC | ARG | A | 457 | 70.830 | −0.789 | 25.989 | 1.00 | 41.99 | C | |
| ATOM | 3549 | CD | ARG | A | 457 | 69.900 | −0.329 | 27.047 | 1.00 | 40.73 | C | |
| ATOM | 3552 | NE | ARG | A | 457 | 70.060 | 1.049 | 27.438 | 1.00 | 39.42 | N | |
| ATOM | 3554 | CZ | ARG | A | 457 | 71.014 | 1.490 | 28.266 | 1.00 | 44.59 | C | |
| ATOM | 3555 | NH1 | ARG | A | 457 | 71.972 | 0.657 | 28.732 | 1.00 | 46.75 | N | |
| ATOM | 3558 | NH2 | ARG | A | 457 | 71.053 | 2.787 | 28.632 | 1.00 | 41.68 | N | |
| ATOM | 3561 | C | ARG | A | 457 | 70.442 | −4.614 | 25.776 | 1.00 | 45.28 | C | |
| ATOM | 3562 | O | ARG | A | 457 | 71.338 | −5.266 | 25.249 | 1.00 | 45.77 | O | |
| ATOM | 3564 | N | MET | A | 458 | 69.174 | −4.996 | 25.824 | 1.00 | 46.74 | N | |
| ATOM | 3565 | CA | MET | A | 458 | 68.673 | −6.223 | 25.276 | 1.00 | 47.04 | C | |
| ATOM | 3567 | CB | MET | A | 458 | 67.153 | −6.327 | 25.495 | 1.00 | 46.68 | C | |
| ATOM | 3570 | CG | MET | A | 458 | 66.718 | −6.782 | 26.905 | 1.00 | 46.91 | C | |
| ATOM | 3573 | SD | MET | A | 458 | 64.868 | −7.030 | 27.067 | 1.00 | 50.39 | S | |
| ATOM | 3574 | CE | MET | A | 458 | 64.286 | −5.185 | 27.361 | 1.00 | 50.07 | C | |
| ATOM | 3578 | C | MET | A | 458 | 68.989 | −6.216 | 23.793 | 1.00 | 47.60 | C | |
| ATOM | 3579 | O | MET | A | 458 | 68.796 | −5.235 | 23.082 | 1.00 | 48.87 | O | |
| ATOM | 3581 | N | GLU | A | 459 | 69.485 | −7.336 | 23.333 | 1.00 | 48.66 | N | |
| ATOM | 3582 | CA | GLU | A | 459 | 69.792 | −7.573 | 21.932 | 1.00 | 48.74 | C | |
| ATOM | 3584 | CB | GLU | A | 459 | 70.420 | −8.984 | 21.789 | 1.00 | 49.12 | C | |
| ATOM | 3587 | CG | GLU | A | 459 | 69.491 | −10.201 | 21.806 | 1.00 | 49.63 | C | |
| ATOM | 3590 | CD | GLU | A | 459 | 68.971 | −10.669 | 23.154 | 1.00 | 51.69 | C | |
| ATOM | 3591 | OE1 | GLU | A | 459 | 68.673 | −9.785 | 23.984 | 1.00 | 51.46 | O | |
| ATOM | 3592 | OE2 | GLU | A | 459 | 68.825 | −11.934 | 23.371 | 1.00 | 51.37 | O | |
| ATOM | 3593 | C | GLU | A | 459 | 68.544 | −7.483 | 21.100 | 1.00 | 48.46 | C | |
| ATOM | 3594 | O | GLU | A | 459 | 67.471 | −7.790 | 21.598 | 1.00 | 47.81 | O | |
| ATOM | 3596 | N | ARG | A | 460 | 68.696 | −7.061 | 19.850 | 1.00 | 48.70 | N | |
| ATOM | 3597 | CA | ARG | A | 460 | 67.624 | −7.146 | 18.850 | 1.00 | 49.66 | C | |
| ATOM | 3599 | CB | ARG | A | 460 | 68.139 | −6.651 | 17.514 | 1.00 | 48.54 | C | |
| ATOM | 3602 | CG | ARG | A | 460 | 67.118 | −6.584 | 16.439 | 1.00 | 51.59 | C | |
| ATOM | 3605 | CD | ARG | A | 460 | 67.704 | −6.694 | 15.030 | 1.00 | 54.46 | C | |
| ATOM | 3608 | NE | ARG | A | 460 | 68.260 | −5.409 | 14.647 | 1.00 | 59.82 | N | |
| ATOM | 3610 | CZ | ARG | A | 460 | 67.883 | −4.620 | 13.628 | 1.00 | 63.54 | C | |
| ATOM | 3611 | NH1 | ARG | A | 460 | 66.931 | −4.991 | 12.762 | 1.00 | 66.66 | N | |
| ATOM | 3614 | NH2 | ARG | A | 460 | 68.518 | −3.451 | 13.450 | 1.00 | 62.38 | N | |
| ATOM | 3617 | C | ARG | A | 460 | 67.017 | −8.594 | 18.728 | 1.00 | 49.50 | C | |
| ATOM | 3618 | O | ARG | A | 460 | 67.725 | −9.574 | 18.394 | 1.00 | 47.71 | O | |
| ATOM | 3620 | N | PRO | A | 461 | 65.701 | −8.719 | 18.985 | 1.00 | 50.24 | N | |
| ATOM | 3621 | CA | PRO | A | 461 | 65.013 | −10.050 | 18.867 | 1.00 | 50.10 | C | |
| ATOM | 3623 | CB | PRO | A | 461 | 63.652 | −9.804 | 19.541 | 1.00 | 50.43 | C | |
| ATOM | 3626 | CG | PRO | A | 461 | 63.397 | −8.320 | 19.350 | 1.00 | 50.15 | C | |
| ATOM | 3629 | CD | PRO | A | 461 | 64.743 | −7.641 | 19.341 | 1.00 | 49.66 | C | |
| ATOM | 3632 | C | PRO | A | 461 | 64.785 | −10.441 | 17.415 | 1.00 | 49.58 | C | |
| ATOM | 3633 | O | PRO | A | 461 | 64.678 | −9.554 | 16.571 | 1.00 | 49.88 | O | |
| ATOM | 3634 | N | GLU | A | 462 | 64.672 | −11.741 | 17.159 | 1.00 | 48.78 | N | |
| ATOM | 3635 | CA | GLU | A | 462 | 64.534 | −12.309 | 15.810 | 1.00 | 48.12 | C | |
| ATOM | 3637 | CB | GLU | A | 462 | 64.095 | −13.784 | 15.879 | 1.00 | 47.84 | C | |
| ATOM | 3640 | CG | GLU | A | 462 | 63.910 | −14.503 | 14.546 | 1.00 | 48.25 | C | |
| ATOM | 3643 | CD | GLU | A | 462 | 65.141 | −14.492 | 13.632 | 1.00 | 49.32 | C | |
| ATOM | 3644 | OE1 | GLU | A | 462 | 66.290 | −14.437 | 14.105 | 1.00 | 49.65 | O | |
| ATOM | 3645 | OE2 | GLU | A | 462 | 64.970 | −14.602 | 12.410 | 1.00 | 52.10 | O | |
| ATOM | 3646 | C | GLU | A | 462 | 63.545 | −11.573 | 14.961 | 1.00 | 47.92 | C | |
| ATOM | 3647 | O | GLU | A | 462 | 62.391 | −11.453 | 15.329 | 1.00 | 47.70 | O | |
| ATOM | 3649 | N | GLY | A | 463 | 64.006 | −11.055 | 13.826 | 1.00 | 48.83 | N | |
| ATOM | 3650 | CA | GLY | A | 463 | 63.104 | −10.503 | 12.810 | 1.00 | 49.16 | C | |
| ATOM | 3653 | C | GLY | A | 463 | 62.495 | −9.157 | 13.095 | 1.00 | 49.48 | C | |
| ATOM | 3654 | O | GLY | A | 463 | 61.742 | −8.664 | 12.283 | 1.00 | 50.32 | O | |
| ATOM | 3656 | N | CYS | A | 464 | 62.818 | −8.550 | 14.225 | 1.00 | 50.06 | N | |
| ATOM | 3657 | CA | CYS | A | 464 | 62.429 | −7.201 | 14.509 | 1.00 | 51.09 | C | |
| ATOM | 3659 | CB | CYS | A | 464 | 63.089 | −6.713 | 15.785 | 1.00 | 50.80 | C | |
| ATOM | 3662 | SG | CYS | A | 464 | 62.533 | −5.049 | 16.177 | 1.00 | 49.81 | S | |
| ATOM | 3664 | C | CYS | A | 464 | 62.907 | −6.325 | 13.382 | 1.00 | 52.38 | C | |
| ATOM | 3665 | O | CYS | A | 464 | 64.061 | −6.463 | 13.051 | 1.00 | 53.60 | O | |
| ATOM | 3667 | N | PRO | A | 465 | 62.074 | −5.486 | 12.753 | 1.00 | 53.51 | N | |
| ATOM | 3668 | CA | PRO | A | 465 | 62.578 | −4.554 | 11.754 | 1.00 | 55.11 | C | |
| ATOM | 3670 | CB | PRO | A | 465 | 61.357 | −3.692 | 11.419 | 1.00 | 54.64 | C | |
| ATOM | 3673 | CG | PRO | A | 465 | 60.228 | −4.579 | 11.639 | 1.00 | 53.39 | C | |
| ATOM | 3676 | CD | PRO | A | 465 | 60.613 | −5.394 | 12.852 | 1.00 | 53.16 | C | |
| ATOM | 3679 | C | PRO | A | 465 | 63.729 | −3.708 | 12.254 | 1.00 | 57.32 | C | |
| ATOM | 3680 | O | PRO | A | 465 | 64.001 | −3.683 | 13.431 | 1.00 | 58.76 | O | |
| ATOM | 3681 | N | GLU | A | 466 | 64.413 | −3.045 | 11.348 | 1.00 | 60.05 | N | |
| ATOM | 3682 | CA | GLU | A | 466 | 65.592 | −2.232 | 11.684 | 1.00 | 61.76 | C | |
| ATOM | 3684 | CB | GLU | A | 466 | 66.490 | −2.066 | 10.413 | 1.00 | 62.21 | C | |
| ATOM | 3687 | CG | GLU | A | 466 | 67.817 | −1.269 | 10.434 | 1.00 | 62.63 | C | |
| ATOM | 3690 | CD | GLU | A | 466 | 68.532 | −1.336 | 9.052 | 1.00 | 65.77 | C | |
| ATOM | 3691 | OE1 | GLU | A | 466 | 67.857 | −1.040 | 8.002 | 1.00 | 70.31 | O | |
| ATOM | 3692 | OE2 | GLU | A | 466 | 69.752 | −1.732 | 8.987 | 1.00 | 70.71 | O | |
| ATOM | 3693 | C | GLU | A | 466 | 65.118 | −0.946 | 12.321 | 1.00 | 61.51 | C | |
| ATOM | 3694 | O | GLU | A | 466 | 65.655 | −0.533 | 13.356 | 1.00 | 61.77 | O | |
| ATOM | 3696 | N | LYS | A | 467 | 64.076 | −0.355 | 11.757 | 1.00 | 61.30 | N | |
| ATOM | 3697 | CA | LYS | A | 467 | 63.540 | 0.870 | 12.320 | 1.00 | 61.66 | C | |
| ATOM | 3699 | CB | LYS | A | 467 | 62.741 | 1.665 | 11.281 | 1.00 | 61.79 | C | |
| ATOM | 3702 | CG | LYS | A | 467 | 63.649 | 2.255 | 10.179 | 1.00 | 63.09 | C | |
| ATOM | 3705 | CD | LYS | A | 467 | 62.947 | 3.410 | 9.409 | 1.00 | 63.74 | C | |
| ATOM | 3708 | CE | LYS | A | 467 | 63.928 | 4.200 | 8.513 | 1.00 | 64.74 | C | |
| ATOM | 3711 | NZ | LYS | A | 467 | 64.544 | 5.376 | 9.218 | 1.00 | 65.98 | N | |
| ATOM | 3715 | C | LYS | A | 467 | 62.781 | 0.620 | 13.632 | 1.00 | 60.39 | C | |
| ATOM | 3716 | O | LYS | A | 467 | 62.692 | 1.547 | 14.467 | 1.00 | 61.04 | O | |
| ATOM | 3718 | N | VAL | A | 468 | 62.310 | −0.614 | 13.869 | 1.00 | 58.38 | N | |
| ATOM | 3719 | CA | VAL | A | 468 | 61.685 | −0.915 | 15.169 | 1.00 | 57.02 | C | |
| ATOM | 3721 | CB | VAL | A | 468 | 60.792 | −2.157 | 15.123 | 1.00 | 56.81 | C | |
| ATOM | 3723 | CG1 | VAL | A | 468 | 60.201 | −2.455 | 16.503 | 1.00 | 56.62 | C | |
| ATOM | 3727 | CG2 | VAL | A | 468 | 59.643 | −1.958 | 14.170 | 1.00 | 56.56 | C | |
| ATOM | 3731 | C | VAL | A | 468 | 62.787 | −0.998 | 16.250 | 1.00 | 56.12 | C | |
| ATOM | 3732 | O | VAL | A | 468 | 62.733 | −0.259 | 17.251 | 1.00 | 55.88 | O | |
| ATOM | 3734 | N | TYR | A | 469 | 63.798 | −1.841 | 16.011 | 1.00 | 54.44 | N | |
| ATOM | 3735 | CA | TYR | A | 469 | 64.979 | −1.846 | 16.850 | 1.00 | 53.72 | C | |
| ATOM | 3737 | CB | TYR | A | 469 | 66.038 | −2.864 | 16.380 | 1.00 | 52.95 | C | |
| ATOM | 3740 | CG | TYR | A | 469 | 67.069 | −3.153 | 17.472 | 1.00 | 51.62 | C | |
| ATOM | 3741 | CD1 | TYR | A | 469 | 66.650 | −3.712 | 18.677 | 1.00 | 51.92 | C | |
| ATOM | 3743 | CE1 | TYR | A | 469 | 67.533 | −3.957 | 19.720 | 1.00 | 51.09 | C | |
| ATOM | 3745 | CZ | TYR | A | 469 | 68.849 | −3.666 | 19.575 | 1.00 | 50.41 | C | |
| ATOM | 3746 | OH | TYR | A | 469 | 69.613 | −3.933 | 20.699 | 1.00 | 50.90 | O | |
| ATOM | 3748 | CE2 | TYR | A | 469 | 69.316 | −3.091 | 18.374 | 1.00 | 49.07 | C | |
| ATOM | 3750 | CD2 | TYR | A | 469 | 68.412 | −2.834 | 17.334 | 1.00 | 47.04 | C | |
| ATOM | 3752 | C | TYR | A | 469 | 65.633 | −0.468 | 17.061 | 1.00 | 53.64 | C | |
| ATOM | 3753 | O | TYR | A | 469 | 65.998 | −0.172 | 18.174 | 1.00 | 53.66 | O | |
| ATOM | 3755 | N | GLU | A | 470 | 65.753 | 0.373 | 16.031 | 1.00 | 54.40 | N | |
| ATOM | 3756 | CA | GLU | A | 470 | 66.277 | 1.780 | 16.199 | 1.00 | 55.39 | C | |
| ATOM | 3758 | CB | GLU | A | 470 | 66.272 | 2.617 | 14.893 | 1.00 | 55.85 | C | |
| ATOM | 3761 | CG | GLU | A | 470 | 67.593 | 2.687 | 14.125 | 1.00 | 58.10 | C | |
| ATOM | 3764 | CD | GLU | A | 470 | 67.420 | 3.018 | 12.619 | 1.00 | 58.70 | C | |
| ATOM | 3765 | OE1 | GLU | A | 470 | 66.577 | 3.927 | 12.274 | 1.00 | 58.60 | O | |
| ATOM | 3766 | OE2 | GLU | A | 470 | 68.148 | 2.351 | 11.796 | 1.00 | 59.32 | O | |
| ATOM | 3767 | C | GLU | A | 470 | 65.473 | 2.570 | 17.217 | 1.00 | 55.05 | C | |
| ATOM | 3768 | O | GLU | A | 470 | 66.053 | 3.318 | 18.005 | 1.00 | 54.93 | O | |
| ATOM | 3770 | N | LEU | A | 471 | 64.142 | 2.429 | 17.156 | 1.00 | 54.69 | N | |
| ATOM | 3771 | CA | LEU | A | 471 | 63.257 | 3.124 | 18.098 | 1.00 | 54.13 | C | |
| ATOM | 3773 | CB | LEU | A | 471 | 61.781 | 3.052 | 17.700 | 1.00 | 54.07 | C | |
| ATOM | 3776 | CG | LEU | A | 471 | 61.326 | 4.034 | 16.619 | 1.00 | 54.83 | C | |
| ATOM | 3778 | CD1 | LEU | A | 471 | 59.808 | 3.915 | 16.513 | 1.00 | 55.39 | C | |
| ATOM | 3782 | CD2 | LEU | A | 471 | 61.703 | 5.518 | 16.875 | 1.00 | 55.64 | C | |
| ATOM | 3786 | C | LEU | A | 471 | 63.425 | 2.612 | 19.506 | 1.00 | 53.27 | C | |
| ATOM | 3787 | O | LEU | A | 471 | 63.479 | 3.443 | 20.421 | 1.00 | 52.70 | O | |
| ATOM | 3789 | N | MET | A | 472 | 63.519 | 1.279 | 19.662 | 1.00 | 52.60 | N | |
| ATOM | 3790 | CA | MET | A | 472 | 63.811 | 0.657 | 20.954 | 1.00 | 52.58 | C | |
| ATOM | 3792 | CB | MET | A | 472 | 64.038 | −0.835 | 20.849 | 1.00 | 52.59 | C | |
| ATOM | 3795 | CG | MET | A | 472 | 62.887 | −1.649 | 20.491 | 1.00 | 52.73 | C | |
| ATOM | 3798 | SD | MET | A | 472 | 63.389 | −3.360 | 20.806 | 1.00 | 54.01 | S | |
| ATOM | 3799 | CE | MET | A | 472 | 62.296 | −4.161 | 19.742 | 1.00 | 53.53 | C | |
| ATOM | 3803 | C | MET | A | 472 | 65.089 | 1.202 | 21.560 | 1.00 | 52.64 | C | |
| ATOM | 3804 | O | MET | A | 472 | 65.135 | 1.516 | 22.752 | 1.00 | 52.03 | O | |
| ATOM | 3806 | N | ARG | A | 473 | 66.131 | 1.294 | 20.747 | 1.00 | 52.93 | N | |
| ATOM | 3807 | CA | ARG | A | 473 | 67.382 | 1.816 | 21.242 | 1.00 | 53.86 | C | |
| ATOM | 3809 | CB | ARG | A | 473 | 68.525 | 1.437 | 20.307 | 1.00 | 54.51 | C | |
| ATOM | 3812 | CG | ARG | A | 473 | 68.768 | −0.075 | 20.223 | 1.00 | 55.83 | C | |
| ATOM | 3815 | CD | ARG | A | 473 | 69.222 | −0.757 | 21.543 | 1.00 | 60.55 | C | |
| ATOM | 3818 | NE | ARG | A | 473 | 70.669 | −1.090 | 21.660 | 1.00 | 63.39 | N | |
| ATOM | 3820 | CZ | ARG | A | 473 | 71.625 | −0.306 | 22.197 | 1.00 | 62.78 | C | |
| ATOM | 3821 | NH1 | ARG | A | 473 | 71.363 | 0.938 | 22.624 | 1.00 | 64.65 | N | |
| ATOM | 3824 | NH2 | ARG | A | 473 | 72.874 | −0.766 | 22.269 | 1.00 | 61.38 | N | |
| ATOM | 3827 | C | ARG | A | 473 | 67.378 | 3.307 | 21.584 | 1.00 | 54.09 | C | |
| ATOM | 3828 | O | ARG | A | 473 | 68.053 | 3.711 | 22.522 | 1.00 | 54.89 | O | |
| ATOM | 3830 | N | ALA | A | 474 | 66.598 | 4.121 | 20.876 | 1.00 | 54.43 | N | |
| ATOM | 3831 | CA | ALA | A | 474 | 66.486 | 5.558 | 21.215 | 1.00 | 54.14 | C | |
| ATOM | 3833 | CB | ALA | A | 474 | 65.930 | 6.381 | 20.037 | 1.00 | 53.93 | C | |
| ATOM | 3837 | C | ALA | A | 474 | 65.608 | 5.702 | 22.459 | 1.00 | 54.26 | C | |
| ATOM | 3838 | O | ALA | A | 474 | 65.831 | 6.604 | 23.308 | 1.00 | 54.45 | O | |
| ATOM | 3840 | N | CYS | A | 475 | 64.615 | 4.814 | 22.568 | 1.00 | 52.81 | N | |
| ATOM | 3841 | CA | CYS | A | 475 | 63.852 | 4.709 | 23.781 | 1.00 | 52.43 | C | |
| ATOM | 3843 | CB | CYS | A | 475 | 62.721 | 3.684 | 23.629 | 1.00 | 53.07 | C | |
| ATOM | 3846 | SG | CYS | A | 475 | 61.280 | 4.398 | 22.870 | 1.00 | 54.23 | S | |
| ATOM | 3848 | C | CYS | A | 475 | 64.705 | 4.340 | 25.016 | 1.00 | 51.73 | C | |
| ATOM | 3849 | O | CYS | A | 475 | 64.288 | 4.615 | 26.144 | 1.00 | 52.56 | O | |
| ATOM | 3851 | N | TRP | A | 476 | 65.851 | 3.692 | 24.835 | 1.00 | 49.72 | N | |
| ATOM | 3852 | CA | TRP | A | 476 | 66.674 | 3.329 | 25.965 | 1.00 | 48.31 | C | |
| ATOM | 3854 | CB | TRP | A | 476 | 67.043 | 1.819 | 25.904 | 1.00 | 47.84 | C | |
| ATOM | 3857 | CG | TRP | A | 476 | 65.866 | 0.886 | 25.808 | 1.00 | 45.66 | C | |
| ATOM | 3858 | CD1 | TRP | A | 476 | 64.620 | 1.116 | 26.282 | 1.00 | 45.95 | C | |
| ATOM | 3860 | NE1 | TRP | A | 476 | 63.814 | 0.034 | 26.041 | 1.00 | 46.25 | N | |
| ATOM | 3862 | CE2 | TRP | A | 476 | 64.538 | −0.930 | 25.406 | 1.00 | 44.25 | C | |
| ATOM | 3863 | CD2 | TRP | A | 476 | 65.846 | −0.416 | 25.236 | 1.00 | 42.39 | C | |
| ATOM | 3864 | CE3 | TRP | A | 476 | 66.796 | −1.208 | 24.586 | 1.00 | 44.51 | C | |
| ATOM | 3866 | CZ3 | TRP | A | 476 | 66.412 | −2.489 | 24.128 | 1.00 | 46.64 | C | |
| ATOM | 3868 | CH2 | TRP | A | 476 | 65.080 | −2.974 | 24.318 | 1.00 | 46.33 | C | |
| ATOM | 3870 | CZ2 | TRP | A | 476 | 64.139 | −2.204 | 24.947 | 1.00 | 45.55 | C | |
| ATOM | 3872 | C | TRP | A | 476 | 67.896 | 4.226 | 26.140 | 1.00 | 48.26 | C | |
| ATOM | 3873 | O | TRP | A | 476 | 68.862 | 3.814 | 26.736 | 1.00 | 47.55 | O | |
| ATOM | 3875 | N | GLN | A | 477 | 67.852 | 5.466 | 25.681 | 1.00 | 48.88 | N | |
| ATOM | 3876 | CA | GLN | A | 477 | 68.919 | 6.393 | 26.022 | 1.00 | 50.02 | C | |
| ATOM | 3878 | CB | GLN | A | 477 | 68.749 | 7.740 | 25.320 | 1.00 | 50.10 | C | |
| ATOM | 3881 | CG | GLN | A | 477 | 68.906 | 7.676 | 23.809 | 1.00 | 51.99 | C | |
| ATOM | 3884 | CD | GLN | A | 477 | 70.272 | 7.187 | 23.334 | 1.00 | 53.01 | C | |
| ATOM | 3885 | OE1 | GLN | A | 477 | 71.301 | 7.563 | 23.864 | 1.00 | 54.29 | O | |
| ATOM | 3886 | NE2 | GLN | A | 477 | 70.264 | 6.348 | 22.325 | 1.00 | 54.64 | N | |
| ATOM | 3889 | C | GLN | A | 477 | 68.982 | 6.608 | 27.560 | 1.00 | 50.32 | C | |
| ATOM | 3890 | O | GLN | A | 477 | 67.965 | 6.807 | 28.212 | 1.00 | 49.81 | O | |
| ATOM | 3892 | N | TRP | A | 478 | 70.191 | 6.483 | 28.115 | 1.00 | 50.78 | N | |
| ATOM | 3893 | CA | TRP | A | 478 | 70.470 | 6.747 | 29.519 | 1.00 | 50.64 | C | |
| ATOM | 3895 | CB | TRP | A | 478 | 71.995 | 6.699 | 29.789 | 1.00 | 49.49 | C | |
| ATOM | 3898 | CG | TRP | A | 478 | 72.350 | 6.853 | 31.240 | 1.00 | 49.47 | C | |
| ATOM | 3899 | CD1 | TRP | A | 478 | 72.749 | 7.999 | 31.875 | 1.00 | 47.77 | C | |
| ATOM | 3901 | NE1 | TRP | A | 478 | 72.938 | 7.747 | 33.212 | 1.00 | 48.01 | N | |
| ATOM | 3903 | CE2 | TRP | A | 478 | 72.645 | 6.435 | 33.477 | 1.00 | 46.76 | C | |
| ATOM | 3904 | CD2 | TRP | A | 478 | 72.277 | 5.834 | 32.259 | 1.00 | 48.94 | C | |
| ATOM | 3905 | CE3 | TRP | A | 478 | 71.951 | 4.457 | 32.257 | 1.00 | 48.50 | C | |
| ATOM | 3907 | CZ3 | TRP | A | 478 | 72.005 | 3.752 | 33.427 | 1.00 | 47.37 | C | |
| ATOM | 3909 | CH2 | TRP | A | 478 | 72.399 | 4.374 | 34.610 | 1.00 | 48.27 | C | |
| ATOM | 3911 | CZ2 | TRP | A | 478 | 72.709 | 5.723 | 34.658 | 1.00 | 47.03 | C | |
| ATOM | 3913 | C | TRP | A | 478 | 69.891 | 8.099 | 29.905 | 1.00 | 52.22 | C | |
| ATOM | 3914 | O | TRP | A | 478 | 69.149 | 8.200 | 30.877 | 1.00 | 53.74 | O | |
| ATOM | 3916 | N | ASN | A | 479 | 70.185 | 9.116 | 29.113 | 1.00 | 53.24 | N | |
| ATOM | 3917 | CA | ASN | A | 479 | 69.860 | 10.481 | 29.463 | 1.00 | 54.64 | C | |
| ATOM | 3919 | CB | ASN | A | 479 | 71.044 | 11.321 | 28.992 | 1.00 | 55.18 | C | |
| ATOM | 3922 | CG | ASN | A | 479 | 70.877 | 12.791 | 29.199 | 1.00 | 58.30 | C | |
| ATOM | 3923 | OD1 | ASN | A | 479 | 69.817 | 13.342 | 29.634 | 1.00 | 63.42 | O | |
| ATOM | 3924 | ND2 | ASN | A | 479 | 71.936 | 13.482 | 28.835 | 1.00 | 62.13 | N | |
| ATOM | 3927 | C | ASN | A | 479 | 68.479 | 10.850 | 28.815 | 1.00 | 54.77 | C | |
| ATOM | 3928 | O | ASN | A | 479 | 68.384 | 10.918 | 27.582 | 1.00 | 54.48 | O | |
| ATOM | 3930 | N | PRO | A | 480 | 67.424 | 11.081 | 29.623 | 1.00 | 54.97 | N | |
| ATOM | 3931 | CA | PRO | A | 480 | 66.022 | 11.238 | 29.086 | 1.00 | 55.15 | C | |
| ATOM | 3933 | CB | PRO | A | 480 | 65.139 | 11.515 | 30.323 | 1.00 | 54.60 | C | |
| ATOM | 3936 | CG | PRO | A | 480 | 66.028 | 11.573 | 31.486 | 1.00 | 54.84 | C | |
| ATOM | 3939 | CD | PRO | A | 480 | 67.456 | 11.187 | 31.086 | 1.00 | 54.86 | C | |
| ATOM | 3942 | C | PRO | A | 480 | 65.790 | 12.298 | 28.022 | 1.00 | 55.19 | C | |
| ATOM | 3943 | O | PRO | A | 480 | 64.904 | 12.137 | 27.195 | 1.00 | 55.66 | O | |
| ATOM | 3944 | N | SER | A | 481 | 66.575 | 13.358 | 28.011 | 1.00 | 55.30 | N | |
| ATOM | 3945 | CA | SER | A | 481 | 66.457 | 14.338 | 26.919 | 1.00 | 55.50 | C | |
| ATOM | 3947 | CB | SER | A | 481 | 67.364 | 15.559 | 27.154 | 1.00 | 55.82 | C | |
| ATOM | 3950 | OG | SER | A | 481 | 68.503 | 15.207 | 27.961 | 1.00 | 58.24 | O | |
| ATOM | 3952 | C | SER | A | 481 | 66.741 | 13.626 | 25.586 | 1.00 | 54.92 | C | |
| ATOM | 3953 | O | SER | A | 481 | 66.020 | 13.823 | 24.623 | 1.00 | 54.70 | O | |
| ATOM | 3955 | N | ASP | A | 482 | 67.719 | 12.717 | 25.587 | 1.00 | 54.83 | N | |
| ATOM | 3956 | CA | ASP | A | 482 | 68.109 | 11.961 | 24.376 | 1.00 | 54.45 | C | |
| ATOM | 3958 | CB | ASP | A | 482 | 69.522 | 11.319 | 24.542 | 1.00 | 54.20 | C | |
| ATOM | 3961 | CG | ASP | A | 482 | 70.635 | 12.372 | 24.904 | 1.00 | 54.75 | C | |
| ATOM | 3962 | OD1 | ASP | A | 482 | 70.452 | 13.571 | 24.551 | 1.00 | 57.92 | O | |
| ATOM | 3963 | OD2 | ASP | A | 482 | 71.704 | 12.132 | 25.541 | 1.00 | 51.96 | O | |
| ATOM | 3964 | C | ASP | A | 482 | 67.031 | 10.981 | 23.872 | 1.00 | 53.70 | C | |
| ATOM | 3965 | O | ASP | A | 482 | 67.035 | 10.625 | 22.736 | 1.00 | 54.26 | O | |
| ATOM | 3967 | N | ARG | A | 483 | 66.082 | 10.592 | 24.696 | 1.00 | 53.87 | N | |
| ATOM | 3968 | CA | ARG | A | 483 | 64.925 | 9.779 | 24.241 | 1.00 | 54.11 | C | |
| ATOM | 3970 | CB | ARG | A | 483 | 64.112 | 9.271 | 25.458 | 1.00 | 53.35 | C | |
| ATOM | 3973 | CG | ARG | A | 483 | 64.577 | 7.925 | 25.921 | 1.00 | 53.98 | C | |
| ATOM | 3976 | CD | ARG | A | 483 | 64.210 | 7.575 | 27.353 | 1.00 | 54.09 | C | |
| ATOM | 3979 | NE | ARG | A | 483 | 65.292 | 7.759 | 28.303 | 1.00 | 52.99 | N | |
| ATOM | 3981 | CZ | ARG | A | 483 | 65.168 | 7.624 | 29.622 | 1.00 | 51.34 | C | |
| ATOM | 3982 | NH1 | ARG | A | 483 | 63.992 | 7.306 | 30.185 | 1.00 | 50.07 | N | |
| ATOM | 3985 | NH2 | ARG | A | 483 | 66.227 | 7.853 | 30.386 | 1.00 | 49.97 | N | |
| ATOM | 3988 | C | ARG | A | 483 | 63.993 | 10.495 | 23.253 | 1.00 | 53.94 | C | |
| ATOM | 3989 | O | ARG | A | 483 | 63.841 | 11.671 | 23.399 | 1.00 | 55.23 | O | |
| ATOM | 3991 | N | PRO | A | 484 | 63.335 | 9.802 | 22.308 | 1.00 | 54.47 | N | |
| ATOM | 3992 | CA | PRO | A | 484 | 62.263 | 10.417 | 21.500 | 1.00 | 54.61 | C | |
| ATOM | 3994 | CB | PRO | A | 484 | 61.876 | 9.309 | 20.488 | 1.00 | 54.75 | C | |
| ATOM | 3997 | CG | PRO | A | 484 | 62.285 | 7.993 | 21.153 | 1.00 | 55.15 | C | |
| ATOM | 4000 | CD | PRO | A | 484 | 63.515 | 8.369 | 21.947 | 1.00 | 55.04 | C | |
| ATOM | 4003 | C | PRO | A | 484 | 61.020 | 10.794 | 22.326 | 1.00 | 55.19 | C | |
| ATOM | 4004 | O | PRO | A | 484 | 60.988 | 10.609 | 23.549 | 1.00 | 55.44 | O | |
| ATOM | 4005 | N | SER | A | 485 | 60.020 | 11.341 | 21.638 | 1.00 | 55.35 | N | |
| ATOM | 4006 | CA | SER | A | 485 | 58.719 | 11.660 | 22.207 | 1.00 | 55.13 | C | |
| ATOM | 4008 | CB | SER | A | 485 | 58.364 | 13.078 | 21.815 | 1.00 | 55.10 | C | |
| ATOM | 4011 | OG | SER | A | 485 | 57.848 | 13.045 | 20.505 | 1.00 | 55.57 | O | |
| ATOM | 4013 | C | SER | A | 485 | 57.676 | 10.711 | 21.624 | 1.00 | 54.79 | C | |
| ATOM | 4014 | O | SER | A | 485 | 57.907 | 10.093 | 20.584 | 1.00 | 53.34 | O | |
| ATOM | 4016 | N | PHE | A | 486 | 56.514 | 10.646 | 22.265 | 1.00 | 54.96 | N | |
| ATOM | 4017 | CA | PHE | A | 486 | 55.416 | 9.819 | 21.747 | 1.00 | 55.83 | C | |
| ATOM | 4019 | CB | PHE | A | 486 | 54.340 | 9.602 | 22.801 | 1.00 | 55.92 | C | |
| ATOM | 4022 | CG | PHE | A | 486 | 54.715 | 8.575 | 23.817 | 1.00 | 56.95 | C | |
| ATOM | 4023 | CD1 | PHE | A | 486 | 54.854 | 7.245 | 23.441 | 1.00 | 55.57 | C | |
| ATOM | 4025 | CE1 | PHE | A | 486 | 55.210 | 6.280 | 24.392 | 1.00 | 56.99 | C | |
| ATOM | 4027 | CZ | PHE | A | 486 | 55.426 | 6.639 | 25.725 | 1.00 | 56.16 | C | |
| ATOM | 4029 | CE2 | PHE | A | 486 | 55.277 | 7.963 | 26.111 | 1.00 | 56.57 | C | |
| ATOM | 4031 | CD2 | PHE | A | 486 | 54.916 | 8.929 | 25.168 | 1.00 | 57.32 | C | |
| ATOM | 4033 | C | PHE | A | 486 | 54.793 | 10.300 | 20.409 | 1.00 | 56.20 | C | |
| ATOM | 4034 | O | PHE | A | 486 | 54.397 | 9.437 | 19.565 | 1.00 | 55.89 | O | |
| ATOM | 4036 | N | ALA | A | 487 | 54.718 | 11.635 | 20.227 | 1.00 | 55.77 | N | |
| ATOM | 4037 | CA | ALA | A | 487 | 54.428 | 12.232 | 18.914 | 1.00 | 55.99 | C | |
| ATOM | 4039 | CB | ALA | A | 487 | 54.392 | 13.755 | 19.001 | 1.00 | 55.56 | C | |
| ATOM | 4043 | C | ALA | A | 487 | 55.441 | 11.749 | 17.837 | 1.00 | 56.19 | C | |
| ATOM | 4044 | O | ALA | A | 487 | 55.019 | 11.195 | 16.816 | 1.00 | 55.87 | O | |
| ATOM | 4046 | N | GLU | A | 488 | 56.751 | 11.888 | 18.112 | 1.00 | 56.63 | N | |
| ATOM | 4047 | CA | GLU | A | 488 | 57.824 | 11.403 | 17.215 | 1.00 | 57.49 | C | |
| ATOM | 4049 | CB | GLU | A | 488 | 59.221 | 11.616 | 17.817 | 1.00 | 57.41 | C | |
| ATOM | 4052 | CG | GLU | A | 488 | 59.751 | 13.040 | 17.893 | 1.00 | 59.42 | C | |
| ATOM | 4055 | CD | GLU | A | 488 | 61.073 | 13.151 | 18.673 | 1.00 | 60.53 | C | |
| ATOM | 4056 | OE1 | GLU | A | 488 | 61.989 | 12.316 | 18.476 | 1.00 | 66.04 | O | |
| ATOM | 4057 | OE2 | GLU | A | 488 | 61.229 | 14.087 | 19.489 | 1.00 | 65.98 | O | |
| ATOM | 4058 | C | GLU | A | 488 | 57.679 | 9.912 | 16.870 | 1.00 | 57.66 | C | |
| ATOM | 4059 | O | GLU | A | 488 | 57.728 | 9.570 | 15.694 | 1.00 | 58.17 | O | |
| ATOM | 4061 | N | ILE | A | 489 | 57.506 | 9.063 | 17.902 | 1.00 | 57.93 | N | |
| ATOM | 4062 | CA | ILE | A | 489 | 57.424 | 7.593 | 17.797 | 1.00 | 57.80 | C | |
| ATOM | 4064 | CB | ILE | A | 489 | 57.316 | 6.917 | 19.216 | 1.00 | 57.97 | C | |
| ATOM | 4066 | CG1 | ILE | A | 489 | 58.635 | 7.023 | 19.992 | 1.00 | 57.93 | C | |
| ATOM | 4069 | CD1 | ILE | A | 489 | 58.506 | 6.904 | 21.514 | 1.00 | 57.02 | C | |
| ATOM | 4073 | CG2 | ILE | A | 489 | 56.900 | 5.422 | 19.139 | 1.00 | 57.66 | C | |
| ATOM | 4077 | C | ILE | A | 489 | 56.219 | 7.191 | 16.997 | 1.00 | 59.21 | C | |
| ATOM | 4078 | O | ILE | A | 489 | 56.345 | 6.328 | 16.117 | 1.00 | 59.18 | O | |
| ATOM | 4080 | N | HIS | A | 490 | 55.048 | 7.766 | 17.333 | 1.00 | 60.68 | N | |
| ATOM | 4081 | CA | HIS | A | 490 | 53.781 | 7.429 | 16.642 | 1.00 | 61.52 | C | |
| ATOM | 4083 | CB | HIS | A | 490 | 52.585 | 8.197 | 17.204 | 1.00 | 61.77 | C | |
| ATOM | 4086 | CG | HIS | A | 490 | 51.278 | 7.876 | 16.521 | 1.00 | 61.77 | C | |
| ATOM | 4087 | ND1 | HIS | A | 490 | 50.864 | 6.581 | 16.273 | 1.00 | 62.15 | N | |
| ATOM | 4089 | CE1 | HIS | A | 490 | 49.683 | 6.601 | 15.685 | 1.00 | 60.38 | C | |
| ATOM | 4091 | NE2 | HIS | A | 490 | 49.315 | 7.857 | 15.545 | 1.00 | 60.73 | N | |
| ATOM | 4093 | CD2 | HIS | A | 490 | 50.294 | 8.677 | 16.052 | 1.00 | 60.01 | C | |
| ATOM | 4095 | C | HIS | A | 490 | 53.876 | 7.716 | 15.150 | 1.00 | 62.34 | C | |
| ATOM | 4096 | O | HIS | A | 490 | 53.542 | 6.847 | 14.332 | 1.00 | 61.47 | O | |
| ATOM | 4098 | N | GLN | A | 491 | 54.343 | 8.936 | 14.842 | 1.00 | 63.75 | N | |
| ATOM | 4099 | CA | GLN | A | 491 | 54.633 | 9.393 | 13.468 | 1.00 | 64.86 | C | |
| ATOM | 4101 | CB | GLN | A | 491 | 55.398 | 10.727 | 13.467 | 1.00 | 64.77 | C | |
| ATOM | 4104 | CG | GLN | A | 491 | 55.596 | 11.400 | 12.095 | 1.00 | 64.80 | C | |
| ATOM | 4107 | CD | GLN | A | 491 | 54.293 | 11.834 | 11.434 | 1.00 | 65.11 | C | |
| ATOM | 4108 | OE1 | GLN | A | 491 | 53.898 | 11.287 | 10.398 | 1.00 | 64.93 | O | |
| ATOM | 4109 | NE2 | GLN | A | 491 | 53.624 | 12.805 | 12.037 | 1.00 | 63.54 | N | |
| ATOM | 4112 | C | GLN | A | 491 | 55.445 | 8.383 | 12.696 | 1.00 | 66.25 | C | |
| ATOM | 4113 | O | GLN | A | 491 | 55.044 | 8.004 | 11.603 | 1.00 | 66.66 | O | |
| ATOM | 4115 | N | ALA | A | 492 | 56.575 | 7.963 | 13.280 | 1.00 | 67.77 | N | |
| ATOM | 4116 | CA | ALA | A | 492 | 57.439 | 6.919 | 12.708 | 1.00 | 68.89 | C | |
| ATOM | 4118 | CB | ALA | A | 492 | 58.673 | 6.692 | 13.561 | 1.00 | 68.89 | C | |
| ATOM | 4122 | C | ALA | A | 492 | 56.705 | 5.603 | 12.509 | 1.00 | 69.86 | C | |
| ATOM | 4123 | O | ALA | A | 492 | 56.892 | 4.962 | 11.477 | 1.00 | 70.02 | O | |
| ATOM | 4125 | N | PHE | A | 493 | 55.871 | 5.223 | 13.479 | 1.00 | 70.90 | N | |
| ATOM | 4126 | CA | PHE | A | 493 | 55.052 | 4.017 | 13.345 | 1.00 | 72.05 | C | |
| ATOM | 4128 | CB | PHE | A | 493 | 54.393 | 3.598 | 14.664 | 1.00 | 71.47 | C | |
| ATOM | 4131 | CG | PHE | A | 493 | 55.226 | 2.669 | 15.461 | 1.00 | 70.61 | C | |
| ATOM | 4132 | CD1 | PHE | A | 493 | 55.283 | 1.326 | 15.114 | 1.00 | 69.27 | C | |
| ATOM | 4134 | CE1 | PHE | A | 493 | 56.062 | 0.460 | 15.824 | 1.00 | 69.40 | C | |
| ATOM | 4136 | CZ | PHE | A | 493 | 56.816 | 0.922 | 16.911 | 1.00 | 69.74 | C | |
| ATOM | 4138 | CE2 | PHE | A | 493 | 56.771 | 2.265 | 17.268 | 1.00 | 69.29 | C | |
| ATOM | 4140 | CD2 | PHE | A | 493 | 55.984 | 3.131 | 16.539 | 1.00 | 69.63 | C | |
| ATOM | 4142 | C | PHE | A | 493 | 53.993 | 4.156 | 12.281 | 1.00 | 72.91 | C | |
| ATOM | 4143 | O | PHE | A | 493 | 53.815 | 3.215 | 11.533 | 1.00 | 72.99 | O | |
| ATOM | 4145 | N | GLU | A | 494 | 53.293 | 5.301 | 12.233 | 1.00 | 74.32 | N | |
| ATOM | 4146 | CA | GLU | A | 494 | 52.277 | 5.592 | 11.184 | 1.00 | 75.23 | C | |
| ATOM | 4148 | CB | GLU | A | 494 | 51.758 | 7.046 | 11.234 | 1.00 | 75.46 | C | |
| ATOM | 4151 | CG | GLU | A | 494 | 50.381 | 7.244 | 11.855 | 1.00 | 75.68 | C | |
| ATOM | 4154 | CD | GLU | A | 494 | 49.858 | 8.655 | 11.601 | 1.00 | 76.06 | C | |
| ATOM | 4155 | OE1 | GLU | A | 494 | 50.272 | 9.603 | 12.328 | 1.00 | 75.88 | O | |
| ATOM | 4156 | OE2 | GLU | A | 494 | 49.044 | 8.822 | 10.652 | 1.00 | 77.47 | O | |
| ATOM | 4157 | C | GLU | A | 494 | 52.866 | 5.328 | 9.809 | 1.00 | 76.90 | C | |
| ATOM | 4158 | O | GLU | A | 494 | 52.349 | 4.485 | 9.082 | 1.00 | 76.96 | O | |
| ATOM | 4160 | N | THR | A | 495 | 53.968 | 6.033 | 9.508 | 1.00 | 78.73 | N | |
| ATOM | 4161 | CA | THR | A | 495 | 54.827 | 5.807 | 8.323 | 1.00 | 80.18 | C | |
| ATOM | 4163 | CB | THR | A | 495 | 56.141 | 6.658 | 8.435 | 1.00 | 79.88 | C | |
| ATOM | 4165 | OG1 | THR | A | 495 | 55.847 | 8.024 | 8.122 | 1.00 | 79.58 | O | |
| ATOM | 4167 | CG2 | THR | A | 495 | 57.216 | 6.267 | 7.395 | 1.00 | 79.47 | C | |
| ATOM | 4171 | C | THR | A | 495 | 55.134 | 4.319 | 8.081 | 1.00 | 80.99 | C | |
| ATOM | 4172 | O | THR | A | 495 | 54.543 | 3.735 | 7.182 | 1.00 | 80.95 | O | |
| ATOM | 4174 | N | MET | A | 496 | 56.009 | 3.722 | 8.904 | 1.00 | 82.49 | N | |
| ATOM | 4175 | CA | MET | A | 496 | 56.347 | 2.263 | 8.864 | 1.00 | 83.49 | C | |
| ATOM | 4177 | CB | MET | A | 496 | 57.035 | 1.823 | 10.172 | 1.00 | 83.57 | C | |
| ATOM | 4180 | CG | MET | A | 496 | 58.482 | 2.231 | 10.294 | 1.00 | 83.65 | C | |
| ATOM | 4183 | SD | MET | A | 496 | 59.290 | 1.292 | 11.589 | 1.00 | 84.99 | S | |
| ATOM | 4184 | CE | MET | A | 496 | 58.899 | 2.235 | 13.040 | 1.00 | 86.07 | C | |
| ATOM | 4188 | C | MET | A | 496 | 55.165 | 1.310 | 8.645 | 1.00 | 84.97 | C | |
| ATOM | 4189 | O | MET | A | 496 | 55.289 | 0.301 | 7.927 | 1.00 | 85.05 | O | |
| ATOM | 4191 | N | PHE | A | 497 | 54.039 | 1.640 | 9.284 | 1.00 | 86.49 | N | |
| ATOM | 4192 | CA | PHE | A | 497 | 52.826 | 0.834 | 9.204 | 1.00 | 87.64 | C | |
| ATOM | 4194 | CB | PHE | A | 497 | 51.818 | 1.203 | 10.304 | 1.00 | 87.70 | C | |
| ATOM | 4197 | CG | PHE | A | 497 | 50.590 | 0.352 | 10.286 | 1.00 | 87.51 | C | |
| ATOM | 4198 | CD1 | PHE | A | 497 | 50.704 | −1.052 | 10.370 | 1.00 | 87.55 | C | |
| ATOM | 4200 | CE1 | PHE | A | 497 | 49.574 | −1.878 | 10.322 | 1.00 | 87.67 | C | |
| ATOM | 4202 | CZ | PHE | A | 497 | 48.303 | −1.297 | 10.189 | 1.00 | 88.27 | C | |
| ATOM | 4204 | CE2 | PHE | A | 497 | 48.180 | 0.121 | 10.097 | 1.00 | 88.29 | C | |
| ATOM | 4206 | CD2 | PHE | A | 497 | 49.328 | 0.927 | 10.138 | 1.00 | 87.42 | C | |
| ATOM | 4208 | C | PHE | A | 497 | 52.143 | 0.876 | 7.832 | 1.00 | 88.50 | C | |
| ATOM | 4209 | O | PHE | A | 497 | 51.702 | −0.175 | 7.344 | 1.00 | 89.13 | O | |
| ATOM | 4211 | N | GLN | A | 498 | 52.045 | 2.055 | 7.217 | 1.00 | 89.15 | N | |
| ATOM | 4212 | CA | GLN | A | 498 | 51.504 | 2.138 | 5.843 | 1.00 | 89.92 | C | |
| ATOM | 4214 | CB | GLN | A | 498 | 51.051 | 3.566 | 5.475 | 1.00 | 89.97 | C | |
| ATOM | 4217 | CG | GLN | A | 498 | 49.986 | 4.196 | 6.436 | 1.00 | 90.20 | C | |
| ATOM | 4220 | CD | GLN | A | 498 | 48.601 | 3.502 | 6.466 | 1.00 | 89.97 | C | |
| ATOM | 4221 | OE1 | GLN | A | 498 | 48.414 | 2.380 | 5.983 | 1.00 | 89.61 | O | |
| ATOM | 4222 | NE2 | GLN | A | 498 | 47.632 | 4.196 | 7.050 | 1.00 | 89.92 | N | |
| ATOM | 4225 | C | GLN | A | 498 | 52.416 | 1.531 | 4.746 | 1.00 | 90.23 | C | |
| ATOM | 4226 | O | GLN | A | 498 | 51.903 | 1.173 | 3.688 | 1.00 | 90.77 | O | |
| ATOM | 4228 | N | OLU | A | 499 | 53.724 | 1.386 | 5.005 | 1.00 | 90.48 | N | |
| ATOM | 4229 | CA | GLU | A | 499 | 54.694 | 0.785 | 4.059 | 1.00 | 90.46 | C | |
| ATOM | 4231 | CB | GLU | A | 499 | 56.057 | 1.479 | 4.186 | 1.00 | 90.46 | C | |
| ATOM | 4234 | CG | GLU | A | 499 | 56.010 | 2.982 | 3.896 | 1.00 | 90.15 | C | |
| ATOM | 4237 | CD | GLU | A | 499 | 57.265 | 3.740 | 4.314 | 1.00 | 89.99 | C | |
| ATOM | 4238 | OE1 | GLU | A | 499 | 58.032 | 3.282 | 5.192 | 1.00 | 89.84 | O | |
| ATOM | 4239 | OE2 | GLU | A | 499 | 57.479 | 4.831 | 3.765 | 1.00 | 88.66 | O | |
| ATOM | 4240 | C | GLU | A | 499 | 54.805 | −0.719 | 4.336 | 1.00 | 90.94 | C | |
| ATOM | 4241 | O | GLU | A | 499 | 55.865 | −1.225 | 4.702 | 1.00 | 91.32 | O | |
| ATOM | 4243 | N | SER | A | 500 | 53.687 | −1.420 | 4.153 | 1.00 | 91.34 | N | |
| ATOM | 4244 | CA | SER | A | 500 | 53.482 | −2.774 | 4.699 | 1.00 | 91.48 | C | |
| ATOM | 4246 | CB | SER | A | 500 | 53.236 | −2.676 | 6.225 | 1.00 | 91.48 | C | |
| ATOM | 4249 | OG | SER | A | 500 | 53.852 | −3.733 | 6.937 | 1.00 | 91.48 | O | |
| ATOM | 4251 | C | SER | A | 500 | 52.331 | −3.501 | 3.947 | 1.00 | 91.62 | C | |
| ATOM | 4252 | O | SER | A | 500 | 51.229 | −3.831 | 4.416 | 1.00 | 91.62 | O | |
| ATOM | 4254 | OXT | SER | A | 500 | 52.495 | −3.764 | 2.756 | 1.00 | 91.74 | O | |
| ATOM | 4255 | N | ASP | B | 233 | 29.222 | 7.771 | 67.736 | 1.00 | 71.87 | N | |
| ATOM | 4256 | CA | ASP | B | 233 | 28.675 | 8.257 | 66.421 | 1.00 | 71.71 | C | |
| ATOM | 4258 | CB | ASP | B | 233 | 28.316 | 9.765 | 66.513 | 1.00 | 71.81 | C | |
| ATOM | 4261 | CG | ASP | B | 233 | 29.506 | 10.691 | 66.271 | 1.00 | 71.64 | C | |
| ATOM | 4262 | OD1 | ASP | B | 233 | 29.858 | 10.923 | 65.103 | 1.00 | 70.89 | O | |
| ATOM | 4263 | OD2 | ASP | B | 233 | 30.123 | 11.274 | 67.177 | 1.00 | 73.18 | O | |
| ATOM | 4264 | C | ASP | B | 233 | 29.599 | 7.910 | 65.212 | 1.00 | 71.33 | C | |
| ATOM | 4265 | O | ASP | B | 233 | 30.555 | 7.140 | 65.357 | 1.00 | 70.95 | O | |
| ATOM | 4269 | N | LYS | B | 234 | 29.252 | 8.464 | 64.046 | 1.00 | 70.98 | N | |
| ATOM | 4270 | CA | LYS | B | 234 | 29.985 | 8.337 | 62.771 | 1.00 | 71.17 | C | |
| ATOM | 4272 | CB | LYS | B | 234 | 29.605 | 9.502 | 61.831 | 1.00 | 71.49 | C | |
| ATOM | 4275 | CG | LYS | B | 234 | 28.140 | 9.548 | 61.391 | 1.00 | 72.11 | C | |
| ATOM | 4278 | CD | LYS | B | 234 | 27.460 | 10.906 | 61.643 | 1.00 | 72.06 | C | |
| ATOM | 4281 | CE | LYS | B | 234 | 25.956 | 10.763 | 61.376 | 1.00 | 72.83 | C | |
| ATOM | 4284 | NZ | LYS | B | 234 | 25.242 | 12.059 | 61.296 | 1.00 | 73.36 | N | |
| ATOM | 4288 | C | LYS | B | 234 | 31.498 | 8.354 | 62.918 | 1.00 | 71.01 | C | |
| ATOM | 4289 | O | LYS | B | 234 | 32.181 | 7.420 | 62.527 | 1.00 | 71.04 | O | |
| ATOM | 4291 | N | TRP | B | 235 | 31.992 | 9.420 | 63.525 | 1.00 | 70.76 | N | |
| ATOM | 4292 | CA | TRP | B | 235 | 33.419 | 9.706 | 63.599 | 1.00 | 70.82 | C | |
| ATOM | 4294 | CB | TRP | B | 235 | 33.635 | 11.196 | 63.929 | 1.00 | 70.12 | C | |
| ATOM | 4297 | CG | TRP | B | 235 | 33.097 | 12.052 | 62.851 | 1.00 | 69.28 | C | |
| ATOM | 4298 | CD1 | TRP | B | 235 | 31.938 | 12.758 | 62.863 | 1.00 | 68.49 | C | |
| ATOM | 4300 | NE1 | TRP | B | 235 | 31.756 | 13.392 | 61.656 | 1.00 | 68.33 | N | |
| ATOM | 4302 | CE2 | TRP | B | 235 | 32.801 | 13.080 | 60.827 | 1.00 | 68.11 | C | |
| ATOM | 4303 | CD2 | TRP | B | 235 | 33.661 | 12.217 | 61.550 | 1.00 | 69.02 | C | |
| ATOM | 4304 | CE3 | TRP | B | 235 | 34.832 | 11.743 | 60.922 | 1.00 | 68.43 | C | |
| ATOM | 4306 | CZ3 | TRP | B | 235 | 35.101 | 12.140 | 59.613 | 1.00 | 68.67 | C | |
| ATOM | 4308 | CH2 | TRP | B | 235 | 34.218 | 13.006 | 58.914 | 1.00 | 68.53 | C | |
| ATOM | 4310 | CZ2 | TRP | B | 235 | 33.070 | 13.483 | 59.504 | 1.00 | 68.14 | C | |
| ATOM | 4312 | C | TRP | B | 235 | 34.155 | 8.840 | 64.604 | 1.00 | 71.63 | C | |
| ATOM | 4313 | O | TRP | B | 235 | 35.295 | 8.455 | 64.374 | 1.00 | 72.01 | O | |
| ATOM | 4315 | N | GLU | B | 236 | 33.495 | 8.523 | 65.711 | 1.00 | 72.93 | N | |
| ATOM | 4316 | CA | GLU | B | 236 | 34.128 | 7.832 | 66.841 | 1.00 | 73.75 | C | |
| ATOM | 4318 | CB | GLU | B | 236 | 33.185 | 7.742 | 68.038 | 1.00 | 73.42 | C | |
| ATOM | 4321 | CG | GLU | B | 236 | 32.846 | 9.116 | 68.630 | 1.00 | 72.96 | C | |
| ATOM | 4324 | CD | GLU | B | 236 | 33.809 | 9.616 | 69.692 | 1.00 | 71.46 | C | |
| ATOM | 4325 | OE1 | GLU | B | 236 | 34.782 | 8.902 | 70.093 | 1.00 | 72.48 | O | |
| ATOM | 4326 | OE2 | GLU | B | 236 | 33.567 | 10.749 | 70.139 | 1.00 | 67.53 | O | |
| ATOM | 4327 | C | GLU | B | 236 | 34.569 | 6.447 | 66.454 | 1.00 | 75.16 | C | |
| ATOM | 4328 | O | GLU | B | 236 | 33.948 | 5.801 | 65.594 | 1.00 | 75.36 | O | |
| ATOM | 4330 | N | MET | B | 237 | 35.651 | 6.005 | 67.092 | 1.00 | 76.54 | N | |
| ATOM | 4331 | CA | MET | B | 237 | 36.283 | 4.738 | 66.758 | 1.00 | 77.67 | C | |
| ATOM | 4333 | CB | MET | B | 237 | 37.071 | 4.936 | 65.480 | 1.00 | 77.70 | C | |
| ATOM | 4336 | CG | MET | B | 237 | 37.915 | 6.168 | 65.480 | 1.00 | 78.27 | C | |
| ATOM | 4339 | SD | MET | B | 237 | 39.363 | 5.931 | 64.441 | 1.00 | 81.61 | S | |
| ATOM | 4340 | CE | MET | B | 237 | 40.430 | 4.806 | 65.532 | 1.00 | 81.78 | C | |
| ATOM | 4344 | C | MET | B | 237 | 37.207 | 4.205 | 67.848 | 1.00 | 77.29 | C | |
| ATOM | 4345 | O | MET | B | 237 | 37.685 | 4.969 | 68.689 | 1.00 | 77.20 | O | |
| ATOM | 4347 | N | GLU | B | 238 | 37.500 | 2.904 | 67.780 | 1.00 | 77.21 | N | |
| ATOM | 4348 | CA | GLU | B | 238 | 38.237 | 2.233 | 68.843 | 1.00 | 77.20 | C | |
| ATOM | 4350 | CB | GLU | B | 238 | 37.967 | 0.721 | 68.835 | 1.00 | 76.95 | C | |
| ATOM | 4353 | CG | GLU | B | 238 | 38.670 | −0.097 | 69.930 | 1.00 | 76.85 | C | |
| ATOM | 4356 | CD | GLU | B | 238 | 38.468 | 0.427 | 71.356 | 1.00 | 75.50 | C | |
| ATOM | 4357 | OE1 | GLU | B | 238 | 37.395 | 0.996 | 71.667 | 1.00 | 73.17 | O | |
| ATOM | 4358 | OE2 | GLU | B | 238 | 39.397 | 0.264 | 72.175 | 1.00 | 74.62 | O | |
| ATOM | 4359 | C | GLU | B | 238 | 39.743 | 2.551 | 68.819 | 1.00 | 77.16 | C | |
| ATOM | 4360 | O | GLU | B | 238 | 40.409 | 2.421 | 67.799 | 1.00 | 76.61 | O | |
| ATOM | 4362 | N | ARG | B | 239 | 40.225 | 2.979 | 69.983 | 1.00 | 77.90 | N | |
| ATOM | 4363 | CA | ARG | B | 239 | 41.631 | 3.341 | 70.292 | 1.00 | 78.53 | C | |
| ATOM | 4365 | CB | ARG | B | 239 | 41.656 | 3.722 | 71.780 | 1.00 | 78.71 | C | |
| ATOM | 4368 | CG | ARG | B | 239 | 42.986 | 4.044 | 72.465 | 1.00 | 79.31 | C | |
| ATOM | 4371 | CD | ARG | B | 239 | 42.787 | 4.405 | 73.935 | 1.00 | 79.93 | C | |
| ATOM | 4374 | NE | ARG | B | 239 | 41.766 | 5.467 | 74.096 | 1.00 | 81.57 | N | |
| ATOM | 4376 | CZ | ARG | B | 239 | 41.979 | 6.735 | 74.493 | 1.00 | 82.84 | C | |
| ATOM | 4377 | NH1 | ARG | B | 239 | 43.206 | 7.169 | 74.840 | 1.00 | 83.83 | N | |
| ATOM | 4380 | NH2 | ARG | B | 239 | 40.941 | 7.572 | 74.584 | 1.00 | 80.98 | N | |
| ATOM | 4383 | C | ARG | B | 239 | 42.687 | 2.256 | 70.029 | 1.00 | 78.60 | C | |
| ATOM | 4384 | O | ARG | B | 239 | 43.789 | 2.557 | 69.564 | 1.00 | 78.18 | O | |
| ATOM | 4386 | N | THR | B | 240 | 42.347 | 1.020 | 70.420 | 1.00 | 79.26 | N | |
| ATOM | 4387 | CA | THR | B | 240 | 43.070 | −0.224 | 70.063 | 1.00 | 79.27 | C | |
| ATOM | 4389 | CB | THR | B | 240 | 42.139 | −1.433 | 70.234 | 1.00 | 79.22 | C | |
| ATOM | 4391 | OG1 | THR | B | 240 | 41.579 | −1.398 | 71.544 | 1.00 | 79.76 | O | |
| ATOM | 4393 | CG2 | THR | B | 240 | 42.890 | −2.790 | 70.153 | 1.00 | 79.35 | C | |
| ATOM | 4397 | C | THR | B | 240 | 43.550 | −0.215 | 68.627 | 1.00 | 79.68 | C | |
| ATOM | 4398 | O | THR | B | 240 | 44.727 | −0.444 | 68.381 | 1.00 | 79.72 | O | |
| ATOM | 4400 | N | ASP | B | 241 | 42.632 | 0.108 | 67.706 | 1.00 | 80.02 | N | |
| ATOM | 4401 | CA | ASP | B | 241 | 42.865 | 0.040 | 66.262 | 1.00 | 80.43 | C | |
| ATOM | 4403 | CB | ASP | B | 241 | 41.699 | 0.654 | 65.456 | 1.00 | 80.82 | C | |
| ATOM | 4406 | CG | ASP | B | 241 | 40.405 | −0.193 | 65.517 | 1.00 | 81.92 | C | |
| ATOM | 4407 | OD1 | ASP | B | 241 | 40.123 | −0.797 | 66.580 | 1.00 | 82.58 | O | |
| ATOM | 4408 | OD2 | ASP | B | 241 | 39.610 | −0.309 | 64.544 | 1.00 | 82.91 | O | |
| ATOM | 4409 | C | ASP | B | 241 | 44.183 | 0.666 | 65.818 | 1.00 | 80.54 | C | |
| ATOM | 4410 | O | ASP | B | 241 | 44.813 | 0.135 | 64.908 | 1.00 | 80.61 | O | |
| ATOM | 4412 | N | ILE | B | 242 | 44.607 | 1.741 | 66.496 | 1.00 | 80.62 | N | |
| ATOM | 4413 | CA | ILE | B | 242 | 45.839 | 2.491 | 66.170 | 1.00 | 80.66 | C | |
| ATOM | 4415 | CB | ILE | B | 242 | 45.614 | 4.029 | 66.403 | 1.00 | 80.79 | C | |
| ATOM | 4417 | CG1 | ILE | B | 242 | 44.297 | 4.533 | 65.785 | 1.00 | 81.46 | C | |
| ATOM | 4420 | CD1 | ILE | B | 242 | 43.630 | 5.535 | 66.665 | 1.00 | 81.89 | C | |
| ATOM | 4424 | CG2 | ILE | B | 242 | 46.771 | 4.852 | 65.839 | 1.00 | 80.63 | C | |
| ATOM | 4428 | C | ILE | B | 242 | 47.105 | 2.038 | 66.972 | 1.00 | 80.54 | C | |
| ATOM | 4429 | O | ILE | B | 242 | 47.154 | 2.213 | 68.202 | 1.00 | 80.22 | O | |
| ATOM | 4431 | N | THR | B | 243 | 48.106 | 1.474 | 66.272 | 1.00 | 80.39 | N | |
| ATOM | 4432 | CA | THR | B | 243 | 49.513 | 1.429 | 66.754 | 1.00 | 80.36 | C | |
| ATOM | 4434 | CB | THR | B | 243 | 50.408 | 0.469 | 65.903 | 1.00 | 80.46 | C | |
| ATOM | 4436 | OG1 | THR | B | 243 | 49.920 | −0.884 | 65.937 | 1.00 | 79.79 | O | |
| ATOM | 4438 | CG2 | THR | B | 243 | 51.839 | 0.364 | 66.481 | 1.00 | 80.52 | C | |
| ATOM | 4442 | C | THR | B | 243 | 50.081 | 2.845 | 66.610 | 1.00 | 80.50 | C | |
| ATOM | 4443 | O | THR | B | 243 | 49.795 | 3.500 | 65.617 | 1.00 | 80.99 | O | |
| ATOM | 4445 | N | MET | B | 244 | 50.900 | 3.291 | 67.570 | 1.00 | 80.35 | N | |
| ATOM | 4446 | CA | MET | B | 244 | 51.532 | 4.632 | 67.560 | 1.00 | 80.01 | C | |
| ATOM | 4448 | CB | MET | B | 244 | 51.321 | 5.307 | 68.899 | 1.00 | 79.71 | C | |
| ATOM | 4451 | CG | MET | B | 244 | 49.874 | 5.491 | 69.311 | 1.00 | 80.04 | C | |
| ATOM | 4454 | SD | MET | B | 244 | 49.308 | 7.231 | 69.362 | 1.00 | 82.18 | S | |
| ATOM | 4455 | CE | MET | B | 244 | 50.604 | 8.042 | 70.563 | 1.00 | 81.97 | C | |
| ATOM | 4459 | C | MET | B | 244 | 53.047 | 4.515 | 67.305 | 1.00 | 79.70 | C | |
| ATOM | 4460 | O | MET | B | 244 | 53.739 | 3.740 | 67.994 | 1.00 | 79.69 | O | |
| ATOM | 4462 | N | LYS | B | 245 | 53.557 | 5.298 | 66.349 | 1.00 | 79.19 | N | |
| ATOM | 4463 | CA | LYS | B | 245 | 54.965 | 5.245 | 65.924 | 1.00 | 78.77 | C | |
| ATOM | 4465 | CB | LYS | B | 245 | 55.083 | 5.254 | 64.393 | 1.00 | 78.72 | C | |
| ATOM | 4468 | CG | LYS | B | 245 | 54.220 | 4.205 | 63.668 | 1.00 | 78.36 | C | |
| ATOM | 4471 | CD | LYS | B | 245 | 54.461 | 4.240 | 62.143 | 1.00 | 78.35 | C | |
| ATOM | 4474 | CE | LYS | B | 245 | 55.620 | 3.359 | 61.703 | 1.00 | 78.65 | C | |
| ATOM | 4477 | NZ | LYS | B | 245 | 55.778 | 3.231 | 60.224 | 1.00 | 78.02 | N | |
| ATOM | 4481 | C | LYS | B | 245 | 55.818 | 6.342 | 66.598 | 1.00 | 78.54 | C | |
| ATOM | 4482 | O | LYS | B | 245 | 56.403 | 6.072 | 67.634 | 1.00 | 79.30 | O | |
| ATOM | 4484 | N | HIS | B | 246 | 55.884 | 7.557 | 66.058 | 1.00 | 78.04 | N | |
| ATOM | 4485 | CA | HIS | B | 246 | 56.888 | 8.554 | 66.501 | 1.00 | 77.74 | C | |
| ATOM | 4487 | CB | HIS | B | 246 | 58.214 | 8.416 | 65.721 | 1.00 | 78.75 | C | |
| ATOM | 4490 | CG | HIS | B | 246 | 58.116 | 8.657 | 64.229 | 1.00 | 82.12 | C | |
| ATOM | 4491 | ND1 HIS | B | 246 | 57.981 | 7.631 | 63.307 | 1.00 | 84.97 | N | | |
| ATOM | 4493 | CE1 | HIS | B | 246 | 57.977 | 8.132 | 62.082 | 1.00 | 84.48 | C | |
| ATOM | 4495 | NE2 | HIS | B | 246 | 58.111 | 9.446 | 62.170 | 1.00 | 85.17 | N | |
| ATOM | 4497 | CD2 | HIS | B | 246 | 58.214 | 9.799 | 63.498 | 1.00 | 84.33 | C | |
| ATOM | 4499 | C | HIS | B | 246 | 56.389 | 9.971 | 66.404 | 1.00 | 76.54 | C | |
| ATOM | 4500 | O | HIS | B | 246 | 55.593 | 10.260 | 65.523 | 1.00 | 75.60 | O | |
| ATOM | 4502 | N | LYS | B | 247 | 56.879 | 10.841 | 67.300 | 1.00 | 75.69 | N | |
| ATOM | 4503 | CA | LYS | B | 247 | 56.505 | 12.289 | 67.328 | 1.00 | 75.38 | C | |
| ATOM | 4505 | CB | LYS | B | 247 | 57.204 | 13.028 | 68.483 | 1.00 | 75.17 | C | |
| ATOM | 4508 | CG | LYS | B | 247 | 56.697 | 14.423 | 68.746 | 1.00 | 74.61 | C | |
| ATOM | 4511 | CD | LYS | B | 247 | 56.926 | 14.830 | 70.189 | 1.00 | 74.47 | C | |
| ATOM | 4514 | CE | LYS | B | 247 | 56.490 | 16.264 | 70.447 | 1.00 | 73.93 | C | |
| ATOM | 4517 | NZ | LYS | B | 247 | 56.085 | 16.466 | 71.853 | 1.00 | 73.65 | N | |
| ATOM | 4521 | C | LYS | B | 247 | 56.852 | 12.983 | 66.024 | 1.00 | 74.82 | C | |
| ATOM | 4522 | O | LYS | B | 247 | 57.855 | 12.626 | 65.394 | 1.00 | 74.48 | O | |
| ATOM | 4524 | N | LEU | B | 248 | 56.029 | 13.966 | 65.638 | 1.00 | 74.77 | N | |
| ATOM | 4525 | CA | LEU | B | 248 | 56.176 | 14.705 | 64.358 | 1.00 | 74.61 | C | |
| ATOM | 4527 | CB | LEU | B | 248 | 54.871 | 14.604 | 63.575 | 1.00 | 74.37 | C | |
| ATOM | 4530 | CG | LEU | B | 248 | 54.555 | 13.219 | 63.021 | 1.00 | 74.17 | C | |
| ATOM | 4532 | CD1 | LEU | B | 248 | 53.143 | 13.189 | 62.457 | 1.00 | 74.25 | C | |
| ATOM | 4536 | CD2 | LEU | B | 248 | 55.567 | 12.800 | 61.935 | 1.00 | 74.91 | C | |
| ATOM | 4540 | C | LEU | B | 248 | 56.598 | 16.170 | 64.510 | 1.00 | 73.91 | C | |
| ATOM | 4541 | O | LEU | B | 248 | 55.987 | 16.901 | 65.265 | 1.00 | 73.45 | O | |
| ATOM | 4543 | N | GLY | B | 254 | 48.936 | 19.328 | 73.181 | 1.00 | 77.99 | N | |
| ATOM | 4544 | CA | GLY | B | 254 | 48.713 | 19.228 | 71.730 | 1.00 | 77.76 | C | |
| ATOM | 4547 | C | GLY | B | 254 | 49.841 | 18.596 | 70.904 | 1.00 | 77.93 | C | |
| ATOM | 4548 | O | GLY | B | 254 | 50.200 | 19.145 | 69.859 | 1.00 | 78.38 | O | |
| ATOM | 4550 | N | GLU | B | 255 | 50.361 | 17.435 | 71.349 | 1.00 | 77.65 | N | |
| ATOM | 4551 | CA | GLU | B | 255 | 51.456 | 16.680 | 70.688 | 1.00 | 77.17 | C | |
| ATOM | 4553 | CB | GLU | B | 255 | 52.171 | 15.730 | 71.676 | 1.00 | 77.20 | C | |
| ATOM | 4556 | CG | GLU | B | 255 | 52.680 | 16.315 | 73.016 | 1.00 | 77.72 | C | |
| ATOM | 4559 | CD | GLU | B | 255 | 52.676 | 15.300 | 74.193 | 1.00 | 77.55 | C | |
| ATOM | 4560 | OE1 | GLU | B | 255 | 52.880 | 14.078 | 73.957 | 1.00 | 76.95 | O | |
| ATOM | 4561 | OE2 | GLU | B | 255 | 52.456 | 15.714 | 75.367 | 1.00 | 76.25 | O | |
| ATOM | 4562 | C | GLU | B | 255 | 50.870 | 15.826 | 69.551 | 1.00 | 77.31 | C | |
| ATOM | 4563 | O | GLU | B | 255 | 49.748 | 15.323 | 69.686 | 1.00 | 78.20 | O | |
| ATOM | 4565 | N | VAL | B | 256 | 51.621 | 15.642 | 68.451 | 1.00 | 76.73 | N | |
| ATOM | 4566 | CA | VAL | B | 256 | 51.184 | 14.832 | 67.265 | 1.00 | 75.57 | C | |
| ATOM | 4568 | CB | VAL | B | 256 | 51.012 | 15.687 | 65.974 | 1.00 | 74.99 | C | |
| ATOM | 4570 | CG1 | VAL | B | 256 | 50.371 | 14.879 | 64.828 | 1.00 | 73.24 | C | |
| ATOM | 4574 | CG2 | VAL | B | 256 | 50.172 | 16.923 | 66.279 | 1.00 | 75.06 | C | |
| ATOM | 4578 | C | VAL | B | 256 | 52.175 | 13.699 | 66.997 | 1.00 | 74.81 | C | |
| ATOM | 4579 | O | VAL | B | 256 | 53.387 | 13.893 | 67.021 | 1.00 | 74.53 | O | |
| ATOM | 4581 | N | TYR | B | 257 | 51.623 | 12.527 | 66.716 | 1.00 | 74.52 | N | |
| ATOM | 4582 | CA | TYR | B | 257 | 52.367 | 11.278 | 66.595 | 1.00 | 74.29 | C | |
| ATOM | 4584 | CB | TYR | B | 257 | 52.022 | 10.342 | 67.773 | 1.00 | 74.06 | C | |
| ATOM | 4587 | CG | TYR | B | 257 | 52.755 | 10.772 | 69.021 | 1.00 | 73.90 | C | |
| ATOM | 4588 | CD1 | TYR | B | 257 | 54.088 | 10.368 | 69.242 | 1.00 | 74.85 | C | |
| ATOM | 4590 | CE1 | TYR | B | 257 | 54.822 | 10.798 | 70.378 | 1.00 | 74.80 | C | |
| ATOM | 4592 | CZ | TYR | B | 257 | 54.214 | 11.662 | 71.303 | 1.00 | 75.29 | C | |
| ATOM | 4593 | OH | TYR | B | 257 | 54.941 | 12.068 | 72.400 | 1.00 | 74.81 | O | |
| ATOM | 4595 | CE2 | TYR | B | 257 | 52.883 | 12.098 | 71.099 | 1.00 | 74.76 | C | |
| ATOM | 4597 | CD2 | TYR | B | 257 | 52.165 | 11.646 | 69.950 | 1.00 | 74.28 | C | |
| ATOM | 4599 | C | TYR | B | 257 | 52.033 | 10.660 | 65.255 | 1.00 | 74.33 | C | |
| ATOM | 4600 | O | TYR | B | 257 | 50.946 | 10.893 | 64.700 | 1.00 | 74.16 | O | |
| ATOM | 4602 | N | GLU | B | 258 | 52.988 | 9.916 | 64.711 | 1.00 | 74.51 | N | |
| ATOM | 4603 | CA | GLU | B | 258 | 52.743 | 9.120 | 63.520 | 1.00 | 74.72 | C | |
| ATOM | 4605 | CB | GLU | B | 258 | 54.028 | 8.801 | 62.730 | 1.00 | 74.77 | C | |
| ATOM | 4608 | CG | GLU | B | 258 | 53.753 | 7.991 | 61.451 | 1.00 | 75.16 | C | |
| ATOM | 4611 | CD | GLU | B | 258 | 54.916 | 7.954 | 60.456 | 1.00 | 75.83 | C | |
| ATOM | 4612 | OE1 | GLU | B | 258 | 55.411 | 9.061 | 60.075 | 1.00 | 74.05 | O | |
| ATOM | 4613 | OE2 | GLU | B | 258 | 55.303 | 6.812 | 60.031 | 1.00 | 77.61 | O | |
| ATOM | 4614 | C | GLU | B | 258 | 52.104 | 7.872 | 64.046 | 1.00 | 74.19 | C | |
| ATOM | 4615 | O | GLU | B | 258 | 52.585 | 7.321 | 65.022 | 1.00 | 74.34 | O | |
| ATOM | 4617 | N | GLY | B | 259 | 51.003 | 7.461 | 63.427 | 1.00 | 74.27 | N | |
| ATOM | 4618 | CA | GLY | B | 259 | 50.267 | 6.251 | 63.816 | 1.00 | 74.05 | C | |
| ATOM | 4621 | C | GLY | B | 259 | 49.906 | 5.405 | 62.615 | 1.00 | 73.92 | C | |
| ATOM | 4622 | O | GLY | B | 259 | 50.067 | 5.826 | 61.467 | 1.00 | 74.07 | O | |
| ATOM | 4624 | N | VAL | B | 260 | 49.407 | 4.215 | 62.910 | 1.00 | 74.01 | N | |
| ATOM | 4625 | CA | VAL | B | 260 | 49.023 | 3.212 | 61.925 | 1.00 | 74.31 | C | |
| ATOM | 4627 | CB | VAL | B | 260 | 49.959 | 1.952 | 61.970 | 1.00 | 73.78 | C | |
| ATOM | 4629 | CG1 | VAL | B | 260 | 49.593 | 0.968 | 60.853 | 1.00 | 73.58 | C | |
| ATOM | 4633 | CG2 | VAL | B | 260 | 51.406 | 2.354 | 61.868 | 1.00 | 73.27 | C | |
| ATOM | 4637 | C | VAL | B | 260 | 47.606 | 2.758 | 62.262 | 1.00 | 75.00 | C | |
| ATOM | 4638 | O | VAL | B | 260 | 47.398 | 2.181 | 63.332 | 1.00 | 74.72 | O | |
| ATOM | 4640 | N | TRP | B | 261 | 46.641 | 3.009 | 61.368 | 1.00 | 75.83 | N | |
| ATOM | 4641 | CA | TRP | B | 261 | 45.303 | 2.381 | 61.462 | 1.00 | 76.03 | C | |
| ATOM | 4643 | CB | TRP | B | 261 | 44.212 | 3.234 | 60.770 | 1.00 | 76.47 | C | |
| ATOM | 4646 | CG | TRP | B | 261 | 42.792 | 2.999 | 61.258 | 1.00 | 76.40 | C | |
| ATOM | 4647 | CD1 | TRP | B | 261 | 42.312 | 1.907 | 61.913 | 1.00 | 77.18 | C | |
| ATOM | 4649 | NE1 | TRP | B | 261 | 40.975 | 2.059 | 62.188 | 1.00 | 76.69 | N | |
| ATOM | 4651 | CE2 | TRP | B | 261 | 40.560 | 3.258 | 61.687 | 1.00 | 76.04 | C | |
| ATOM | 4652 | CD2 | TRP | B | 261 | 41.679 | 3.875 | 61.091 | 1.00 | 76.69 | C | |
| ATOM | 4653 | CE3 | TRP | B | 261 | 41.512 | 5.139 | 60.508 | 1.00 | 78.52 | C | |
| ATOM | 4655 | CZ3 | TRP | B | 261 | 40.223 | 5.742 | 60.534 | 1.00 | 78.10 | C | |
| ATOM | 4657 | CH2 | TRP | B | 261 | 39.135 | 5.081 | 61.127 | 1.00 | 77.12 | C | |
| ATOM | 4659 | CZ2 | TRP | B | 261 | 39.287 | 3.851 | 61.712 | 1.00 | 76.77 | C | |
| ATOM | 4661 | C | TRP | B | 261 | 45.418 | 0.944 | 60.893 | 1.00 | 76.08 | C | |
| ATOM | 4662 | O | TRP | B | 261 | 45.013 | 0.658 | 59.755 | 1.00 | 76.05 | O | |
| ATOM | 4664 | N | LYS | B | 262 | 46.005 | 0.073 | 61.733 | 1.00 | 76.08 | N | |
| ATOM | 4665 | CA | LYS | B | 262 | 46.209 | −1.384 | 61.527 | 1.00 | 75.79 | C | |
| ATOM | 4667 | CB | LYS | B | 262 | 46.218 | −2.122 | 62.894 | 1.00 | 75.92 | C | |
| ATOM | 4670 | CG | LYS | B | 262 | 47.274 | −1.763 | 63.996 | 1.00 | 75.88 | C | |
| ATOM | 4673 | CD | LYS | B | 262 | 47.091 | −2.742 | 65.220 | 1.00 | 76.26 | C | |
| ATOM | 4676 | CE | LYS | B | 262 | 47.015 | −2.075 | 66.614 | 1.00 | 76.17 | C | |
| ATOM | 4679 | NZ | LYS | B | 262 | 46.269 | −2.948 | 67.589 | 1.00 | 76.29 | N | |
| ATOM | 4683 | C | LYS | B | 262 | 45.125 | −2.063 | 60.666 | 1.00 | 75.58 | C | |
| ATOM | 4684 | O | LYS | B | 262 | 45.442 | −2.780 | 59.700 | 1.00 | 75.22 | O | |
| ATOM | 4686 | N | LYS | B | 263 | 43.862 | −1.809 | 61.058 | 1.00 | 75.43 | N | |
| ATOM | 4687 | CA | LYS | B | 263 | 42.610 | −2.343 | 60.434 | 1.00 | 75.22 | C | |
| ATOM | 4689 | CB | LYS | B | 263 | 41.371 | −1.672 | 61.094 | 1.00 | 75.29 | C | |
| ATOM | 4692 | CG | LYS | B | 263 | 39.988 | −1.938 | 60.457 | 1.00 | 75.46 | C | |
| ATOM | 4695 | CD | LYS | B | 263 | 38.810 | −1.709 | 61.457 | 1.00 | 75.52 | C | |
| ATOM | 4698 | CE | LYS | B | 263 | 38.634 | −2.877 | 62.459 | 1.00 | 76.30 | C | |
| ATOM | 4701 | NZ | LYS | B | 263 | 37.203 | −3.292 | 62.682 | 1.00 | 76.12 | N | |
| ATOM | 4705 | C | LYS | B | 263 | 42.554 | −2.148 | 58.918 | 1.00 | 74.43 | C | |
| ATOM | 4706 | O | LYS | B | 263 | 42.212 | −3.085 | 58.170 | 1.00 | 74.18 | O | |
| ATOM | 4708 | N | TYR | B | 264 | 42.923 | −0.936 | 58.497 | 1.00 | 73.31 | N | |
| ATOM | 4709 | CA | TYR | B | 264 | 42.941 | −0.541 | 57.097 | 1.00 | 72.28 | C | |
| ATOM | 4711 | CB | TYR | B | 264 | 42.196 | 0.785 | 56.974 | 1.00 | 73.54 | C | |
| ATOM | 4714 | CG | TYR | B | 264 | 40.779 | 0.744 | 57.526 | 1.00 | 74.52 | C | |
| ATOM | 4715 | CD1 | TYR | B | 264 | 39.743 | 0.084 | 56.831 | 1.00 | 75.51 | C | |
| ATOM | 4717 | CE1 | TYR | B | 264 | 38.422 | 0.065 | 57.338 | 1.00 | 74.65 | C | |
| ATOM | 4719 | CZ | TYR | B | 264 | 38.142 | 0.697 | 58.542 | 1.00 | 74.09 | C | |
| ATOM | 4720 | OH | TYR | B | 264 | 36.883 | 0.723 | 59.078 | 1.00 | 74.39 | O | |
| ATOM | 4722 | CE2 | TYR | B | 264 | 39.139 | 1.343 | 59.240 | 1.00 | 74.81 | C | |
| ATOM | 4724 | CD2 | TYR | B | 264 | 40.458 | 1.374 | 58.733 | 1.00 | 75.16 | C | |
| ATOM | 4726 | C | TYR | B | 264 | 44.353 | −0.432 | 56.468 | 1.00 | 71.56 | C | |
| ATOM | 4727 | O | TYR | B | 264 | 44.480 | 0.121 | 55.358 | 1.00 | 71.76 | O | |
| ATOM | 4729 | N | SER | B | 265 | 45.389 | −0.954 | 57.146 | 1.00 | 69.99 | N | |
| ATOM | 4730 | CA | SER | B | 265 | 46.770 | −0.987 | 56.631 | 1.00 | 68.92 | C | |
| ATOM | 4732 | CB | SER | B | 265 | 46.847 | −2.022 | 55.504 | 1.00 | 68.35 | C | |
| ATOM | 4735 | OG | SER | B | 265 | 48.186 | −2.323 | 55.159 | 1.00 | 67.80 | O | |
| ATOM | 4737 | C | SER | B | 265 | 47.263 | 0.429 | 56.192 | 1.00 | 67.95 | C | |
| ATOM | 4738 | O | SER | B | 265 | 47.826 | 0.605 | 55.105 | 1.00 | 67.21 | O | |
| ATOM | 4740 | N | LEU | B | 266 | 47.037 | 1.414 | 57.072 | 1.00 | 67.13 | N | |
| ATOM | 4741 | CA | LEU | B | 266 | 47.051 | 2.855 | 56.724 | 1.00 | 67.09 | C | |
| ATOM | 4743 | CB | LEU | B | 266 | 45.598 | 3.341 | 56.615 | 1.00 | 66.89 | C | |
| ATOM | 4746 | CG | LEU | B | 266 | 45.310 | 4.839 | 56.477 | 1.00 | 66.36 | C | |
| ATOM | 4748 | CD1 | LEU | B | 266 | 45.916 | 5.323 | 55.221 | 1.00 | 66.40 | C | |
| ATOM | 4752 | CD2 | LEU | B | 266 | 43.815 | 5.120 | 56.465 | 1.00 | 66.34 | C | |
| ATOM | 4756 | C | LEU | B | 266 | 47.812 | 3.731 | 57.747 | 1.00 | 66.82 | C | |
| ATOM | 4757 | O | LEU | B | 266 | 47.629 | 3.568 | 58.948 | 1.00 | 66.59 | O | |
| ATOM | 4759 | N | THR | B | 267 | 48.626 | 4.669 | 57.255 | 1.00 | 66.40 | N | |
| ATOM | 4760 | CA | THR | B | 267 | 49.396 | 5.604 | 58.101 | 1.00 | 66.25 | C | |
| ATOM | 4762 | CB | THR | B | 267 | 50.786 | 5.892 | 57.476 | 1.00 | 66.40 | C | |
| ATOM | 4764 | OG1 | THR | B | 267 | 51.426 | 4.657 | 57.129 | 1.00 | 66.77 | O | |
| ATOM | 4766 | CG2 | THR | B | 267 | 51.754 | 6.539 | 58.494 | 1.00 | 66.42 | C | |
| ATOM | 4770 | C | THR | B | 267 | 48.657 | 6.926 | 58.333 | 1.00 | 65.66 | C | |
| ATOM | 4771 | O | THR | B | 267 | 48.231 | 7.601 | 57.388 | 1.00 | 65.10 | O | |
| ATOM | 4773 | N | VAL | B | 268 | 48.562 | 7.303 | 59.599 | 1.00 | 65.42 | N | |
| ATOM | 4774 | CA | VAL | B | 268 | 47.797 | 8.470 | 60.027 | 1.00 | 65.52 | C | |
| ATOM | 4776 | CB | VAL | B | 268 | 46.483 | 8.090 | 60.724 | 1.00 | 65.19 | C | |
| ATOM | 4778 | CG1 | VAL | B | 268 | 45.506 | 7.529 | 59.714 | 1.00 | 64.85 | C | |
| ATOM | 4782 | CG2 | VAL | B | 268 | 46.706 | 7.085 | 61.876 | 1.00 | 65.55 | C | |
| ATOM | 4786 | C | VAL | B | 268 | 48.601 | 9.325 | 60.977 | 1.00 | 66.07 | C | |
| ATOM | 4787 | O | VAL | B | 268 | 49.564 | 8.849 | 61.611 | 1.00 | 66.16 | O | |
| ATOM | 4789 | N | ALA | B | 269 | 48.193 | 10.590 | 61.047 | 1.00 | 66.44 | N | |
| ATOM | 4790 | CA | ALA | B | 269 | 48.684 | 11.538 | 62.023 | 1.00 | 66.73 | C | |
| ATOM | 4792 | CB | ALA | B | 269 | 48.835 | 12.899 | 61.412 | 1.00 | 66.48 | C | |
| ATOM | 4796 | C | ALA | B | 269 | 47.637 | 11.547 | 63.141 | 1.00 | 67.56 | C | |
| ATOM | 4797 | O | ALA | B | 269 | 46.419 | 11.624 | 62.889 | 1.00 | 66.88 | O | |
| ATOM | 4799 | N | VAL | B | 270 | 48.122 | 11.464 | 64.373 | 1.00 | 68.46 | N | |
| ATOM | 4800 | CA | VAL | B | 270 | 47.264 | 11.350 | 65.529 | 1.00 | 69.21 | C | |
| ATOM | 4802 | CB | VAL | B | 270 | 47.498 | 10.020 | 66.294 | 1.00 | 69.47 | C | |
| ATOM | 4804 | CG1 | VAL | B | 270 | 46.556 | 9.924 | 67.520 | 1.00 | 70.10 | C | |
| ATOM | 4808 | CG2 | VAL | B | 270 | 47.285 | 8.833 | 65.354 | 1.00 | 68.98 | C | |
| ATOM | 4812 | C | VAL | B | 270 | 47.598 | 12.513 | 66.388 | 1.00 | 69.41 | C | |
| ATOM | 4813 | O | VAL | B | 270 | 48.689 | 12.566 | 66.916 | 1.00 | 68.78 | O | |
| ATOM | 4815 | N | LYS | B | 271 | 46.672 | 13.457 | 66.486 | 1.00 | 70.93 | N | |
| ATOM | 4816 | CA | LYS | B | 271 | 46.834 | 14.594 | 67.389 | 1.00 | 72.33 | C | |
| ATOM | 4818 | CB | LYS | B | 271 | 46.197 | 15.881 | 66.872 | 1.00 | 71.32 | C | |
| ATOM | 4821 | CG | LYS | B | 271 | 46.305 | 16.998 | 67.905 | 1.00 | 71.61 | C | |
| ATOM | 4824 | CD | LYS | B | 271 | 46.396 | 18.372 | 67.267 | 1.00 | 71.68 | C | |
| ATOM | 4827 | CE | LYS | B | 271 | 46.395 | 19.461 | 68.330 | 1.00 | 69.12 | C | |
| ATOM | 4830 | NZ | LYS | B | 271 | 45.648 | 20.556 | 67.814 | 1.00 | 64.94 | N | |
| ATOM | 4834 | C | LYS | B | 271 | 46.258 | 14.187 | 68.734 | 1.00 | 73.84 | C | |
| ATOM | 4835 | O | LYS | B | 271 | 45.140 | 13.710 | 68.799 | 1.00 | 73.97 | O | |
| ATOM | 4837 | N | THR | B | 272 | 47.036 | 14.426 | 69.786 | 1.00 | 76.02 | N | |
| ATOM | 4838 | CA | THR | B | 272 | 46.780 | 13.915 | 71.107 | 1.00 | 77.93 | C | |
| ATOM | 4840 | CB | THR | B | 272 | 47.803 | 12.776 | 71.379 | 1.00 | 78.19 | C | |
| ATOM | 4842 | OG1 | THR | B | 272 | 47.194 | 11.783 | 72.221 | 1.00 | 79.95 | O | |
| ATOM | 4844 | CG2 | THR | B | 272 | 49.100 | 13.238 | 72.131 | 1.00 | 77.87 | C | |
| ATOM | 4848 | C | THR | B | 272 | 46.816 | 15.027 | 72.165 | 1.00 | 79.55 | C | |
| ATOM | 4849 | O | THR | B | 272 | 47.117 | 16.181 | 71.858 | 1.00 | 79.69 | O | |
| ATOM | 4851 | N | LEU | B | 273 | 46.512 | 14.660 | 73.408 | 1.00 | 81.73 | N | |
| ATOM | 4852 | CA | LEU | B | 273 | 46.434 | 15.622 | 74.511 | 1.00 | 83.33 | C | |
| ATOM | 4854 | CB | LEU | B | 273 | 44.963 | 15.851 | 74.901 | 1.00 | 83.12 | C | |
| ATOM | 4857 | CG | LEU | B | 273 | 44.548 | 17.212 | 75.476 | 1.00 | 82.38 | C | |
| ATOM | 4859 | CD1 | LEU | B | 273 | 45.077 | 18.405 | 74.660 | 1.00 | 82.39 | C | |
| ATOM | 4863 | CD2 | LEU | B | 273 | 43.022 | 17.264 | 75.584 | 1.00 | 82.43 | C | |
| ATOM | 4867 | C | LEU | B | 273 | 47.290 | 15.181 | 75.720 | 1.00 | 84.40 | C | |
| ATOM | 4868 | O | LEU | B | 273 | 47.252 | 13.984 | 76.120 | 1.00 | 84.36 | O | |
| ATOM | 4870 | N | LYS | B | 274 | 48.053 | 16.167 | 76.255 | 1.00 | 85.64 | N | |
| ATOM | 4871 | CA | LYS | B | 274 | 48.922 | 16.054 | 77.454 | 1.00 | 86.47 | C | |
| ATOM | 4873 | CB | LYS | B | 274 | 49.579 | 17.415 | 77.801 | 1.00 | 86.83 | C | |
| ATOM | 4876 | CG | LYS | B | 274 | 50.553 | 18.003 | 76.747 | 1.00 | 87.38 | C | |
| ATOM | 4879 | CD | LYS | B | 274 | 50.590 | 19.553 | 76.777 | 1.00 | 87.23 | C | |
| ATOM | 4882 | CE | LYS | B | 274 | 51.404 | 20.146 | 75.618 | 1.00 | 87.22 | C | |
| ATOM | 4885 | NZ | LYS | B | 274 | 51.299 | 21.633 | 75.568 | 1.00 | 86.43 | N | |
| ATOM | 4889 | C | LYS | B | 274 | 48.070 | 15.607 | 78.647 | 1.00 | 87.79 | C | |
| ATOM | 4890 | O | LYS | B | 274 | 47.018 | 16.208 | 78.896 | 1.00 | 87.91 | O | |
| ATOM | 4892 | N | GLU | B | 275 | 48.562 | 14.617 | 79.399 | 1.00 | 88.93 | N | |
| ATOM | 4893 | CA | GLU | B | 275 | 47.720 | 13.719 | 80.238 | 1.00 | 90.06 | C | |
| ATOM | 4895 | CB | GLU | B | 275 | 48.584 | 12.678 | 81.008 | 1.00 | 90.30 | C | |
| ATOM | 4898 | CG | GLU | B | 275 | 49.502 | 11.795 | 80.147 | 1.00 | 91.26 | C | |
| ATOM | 4901 | CD | GLU | B | 275 | 48.806 | 11.208 | 78.923 | 1.00 | 92.58 | C | |
| ATOM | 4902 | OE1 | GLU | B | 275 | 48.158 | 10.143 | 79.080 | 1.00 | 94.14 | O | |
| ATOM | 4903 | OE2 | GLU | B | 275 | 48.886 | 11.823 | 77.821 | 1.00 | 91.41 | O | |
| ATOM | 4904 | C | GLU | B | 275 | 46.730 | 14.404 | 81.206 | 1.00 | 90.45 | C | |
| ATOM | 4905 | O | GLU | B | 275 | 47.062 | 14.690 | 82.368 | 1.00 | 90.70 | O | |
| ATOM | 4907 | N | ASP | B | 276 | 45.527 | 14.681 | 80.675 | 1.00 | 90.77 | N | |
| ATOM | 4908 | CA | ASP | B | 276 | 44.353 | 15.163 | 81.429 | 1.00 | 90.66 | C | |
| ATOM | 4910 | CB | ASP | B | 276 | 43.916 | 14.107 | 82.472 | 1.00 | 90.82 | C | |
| ATOM | 4913 | CG | ASP | B | 276 | 43.807 | 12.673 | 81.872 | 1.00 | 91.31 | C | |
| ATOM | 4914 | OD1 | ASP | B | 276 | 43.534 | 12.535 | 80.653 | 1.00 | 92.61 | O | |
| ATOM | 4915 | OD2 | ASP | B | 276 | 43.986 | 11.629 | 82.541 | 1.00 | 90.49 | O | |
| ATOM | 4916 | C | ASP | B | 276 | 44.575 | 16.578 | 82.019 | 1.00 | 90.66 | C | |
| ATOM | 4917 | O | ASP | B | 276 | 44.516 | 16.787 | 83.237 | 1.00 | 90.58 | O | |
| ATOM | 4919 | N | THR | B | 277 | 44.827 | 17.528 | 81.110 | 1.00 | 90.64 | N | |
| ATOM | 4920 | CA | THR | B | 277 | 45.103 | 18.950 | 81.415 | 1.00 | 90.55 | C | |
| ATOM | 4922 | CB | THR | B | 277 | 46.322 | 19.454 | 80.510 | 1.00 | 90.81 | C | |
| ATOM | 4924 | OG1 | THR | B | 277 | 47.362 | 18.465 | 80.488 | 1.00 | 91.55 | O | |
| ATOM | 4926 | CG2 | THR | B | 277 | 47.070 | 20.675 | 81.072 | 1.00 | 90.67 | C | |
| ATOM | 4930 | C | THR | B | 277 | 43.803 | 19.778 | 81.191 | 1.00 | 90.34 | C | |
| ATOM | 4931 | O | THR | B | 277 | 42.723 | 19.209 | 80.980 | 1.00 | 90.06 | O | |
| ATOM | 4933 | N | MET | B | 278 | 43.915 | 21.106 | 81.303 | 1.00 | 90.13 | N | |
| ATOM | 4934 | CA | MET | B | 278 | 42.851 | 22.098 | 80.958 | 1.00 | 89.77 | C | |
| ATOM | 4936 | CB | MET | B | 278 | 43.210 | 23.466 | 81.571 | 1.00 | 89.80 | C | |
| ATOM | 4939 | CG | MET | B | 278 | 44.410 | 24.181 | 80.906 | 1.00 | 90.14 | C | |
| ATOM | 4942 | SD | MET | B | 278 | 44.666 | 25.819 | 81.565 | 1.00 | 91.00 | S | |
| ATOM | 4943 | CE | MET | B | 278 | 43.366 | 26.812 | 80.752 | 1.00 | 91.23 | C | |
| ATOM | 4947 | C | MET | B | 278 | 42.516 | 22.348 | 79.460 | 1.00 | 89.24 | C | |
| ATOM | 4948 | O | MET | B | 278 | 41.521 | 23.028 | 79.154 | 1.00 | 89.00 | O | |
| ATOM | 4950 | N | GLU | B | 279 | 43.325 | 21.817 | 78.537 | 1.00 | 88.58 | N | |
| ATOM | 4951 | CA | GLU | B | 279 | 43.235 | 22.154 | 77.102 | 1.00 | 87.74 | C | |
| ATOM | 4953 | CB | GLU | B | 279 | 44.604 | 21.859 | 76.411 | 1.00 | 87.95 | C | |
| ATOM | 4956 | CG | GLU | B | 279 | 45.829 | 22.642 | 76.924 | 1.00 | 88.19 | C | |
| ATOM | 4959 | CD | GLU | B | 279 | 47.176 | 21.917 | 76.715 | 1.00 | 88.45 | C | |
| ATOM | 4960 | OE1 | GLU | B | 279 | 47.477 | 21.432 | 75.584 | 1.00 | 89.31 | O | |
| ATOM | 4961 | OE2 | GLU | B | 279 | 47.952 | 21.826 | 77.703 | 1.00 | 89.33 | O | |
| ATOM | 4962 | C | GLU | B | 279 | 42.078 | 21.407 | 76.373 | 1.00 | 86.89 | C | |
| ATOM | 4963 | O | GLU | B | 279 | 42.118 | 21.256 | 75.144 | 1.00 | 87.26 | O | |
| ATOM | 4965 | N | VAL | B | 280 | 41.025 | 21.002 | 77.092 | 1.00 | 85.58 | N | |
| ATOM | 4966 | CA | VAL | B | 280 | 40.112 | 19.954 | 76.618 | 1.00 | 84.75 | C | |
| ATOM | 4968 | CB | VAL | B | 280 | 39.455 | 19.139 | 77.767 | 1.00 | 84.63 | C | |
| ATOM | 4970 | CG1 | VAL | B | 280 | 38.671 | 17.938 | 77.194 | 1.00 | 84.05 | C | |
| ATOM | 4974 | CG2 | VAL | B | 280 | 40.502 | 18.668 | 78.779 | 1.00 | 84.77 | C | |
| ATOM | 4978 | C | VAL | B | 280 | 39.015 | 20.539 | 75.764 | 1.00 | 82.91 | C | |
| ATOM | 4979 | O | VAL | B | 280 | 38.838 | 20.110 | 74.644 | 1.00 | 82.80 | O | |
| ATOM | 4981 | N | GLU | B | 281 | 38.266 | 21.496 | 76.304 | 1.00 | 81.34 | N | |
| ATOM | 4982 | CA | GLU | B | 281 | 37.148 | 22.118 | 75.568 | 1.00 | 79.94 | C | |
| ATOM | 4984 | CB | GLU | B | 281 | 36.496 | 23.257 | 76.373 | 1.00 | 79.79 | C | |
| ATOM | 4987 | CG | GLU | B | 281 | 35.821 | 22.849 | 77.687 | 1.00 | 79.41 | C | |
| ATOM | 4990 | CD | GLU | B | 281 | 34.421 | 22.232 | 77.553 | 1.00 | 78.70 | C | |
| ATOM | 4991 | OE1 | GLU | B | 281 | 34.014 | 21.806 | 76.454 | 1.00 | 78.74 | O | |
| ATOM | 4992 | OE2 | GLU | B | 281 | 33.715 | 22.153 | 78.584 | 1.00 | 77.12 | O | |
| ATOM | 4993 | C | GLU | B | 281 | 37.597 | 22.644 | 74.197 | 1.00 | 79.36 | C | |
| ATOM | 4994 | O | GLU | B | 281 | 36.825 | 22.579 | 73.251 | 1.00 | 79.15 | O | |
| ATOM | 4996 | N | GLU | B | 282 | 38.840 | 23.145 | 74.124 | 1.00 | 78.53 | N | |
| ATOM | 4997 | CA | GLU | B | 282 | 39.519 | 23.584 | 72.877 | 1.00 | 78.25 | C | |
| ATOM | 4999 | CB | GLU | B | 282 | 40.877 | 24.243 | 73.215 | 1.00 | 78.32 | C | |
| ATOM | 5002 | CG | GLU | B | 282 | 40.768 | 25.700 | 73.658 | 1.00 | 80.39 | C | |
| ATOM | 5005 | CD | GLU | B | 282 | 40.198 | 26.611 | 72.569 | 1.00 | 84.05 | C | |
| ATOM | 5006 | OE1 | GLU | B | 282 | 40.394 | 26.303 | 71.354 | 1.00 | 86.18 | O | |
| ATOM | 5007 | OE2 | GLU | B | 282 | 39.527 | 27.618 | 72.925 | 1.00 | 85.80 | O | |
| ATOM | 5008 | C | GLU | B | 282 | 39.755 | 22.500 | 71.813 | 1.00 | 76.92 | C | |
| ATOM | 5009 | O | GLU | B | 282 | 39.569 | 22.740 | 70.621 | 1.00 | 76.75 | O | |
| ATOM | 5011 | N | PHE | B | 283 | 40.224 | 21.342 | 72.268 | 1.00 | 75.59 | N | |
| ATOM | 5012 | CA | PHE | B | 283 | 40.312 | 20.102 | 71.481 | 1.00 | 74.41 | C | |
| ATOM | 5014 | CB | PHE | B | 283 | 40.786 | 18.973 | 72.403 | 1.00 | 74.84 | C | |
| ATOM | 5017 | CG | PHE | B | 283 | 41.629 | 17.962 | 71.740 | 1.00 | 75.42 | C | |
| ATOM | 5018 | CD1 | PHE | B | 283 | 42.965 | 18.240 | 71.470 | 1.00 | 76.41 | C | |
| ATOM | 5020 | CE1 | PHE | B | 283 | 43.790 | 17.273 | 70.855 | 1.00 | 76.45 | C | |
| ATOM | 5022 | CZ | PHE | B | 283 | 43.269 | 16.026 | 70.522 | 1.00 | 76.08 | C | |
| ATOM | 5024 | CE2 | PHE | B | 283 | 41.937 | 15.729 | 70.799 | 1.00 | 76.63 | C | |
| ATOM | 5026 | CD2 | PHE | B | 283 | 41.119 | 16.699 | 71.419 | 1.00 | 77.17 | C | |
| ATOM | 5028 | C | PHE | B | 283 | 38.954 | 19.714 | 70.859 | 1.00 | 73.30 | C | |
| ATOM | 5029 | O | PHE | B | 283 | 38.866 | 19.408 | 69.674 | 1.00 | 73.03 | O | |
| ATOM | 5031 | N | LEU | B | 284 | 37.902 | 19.754 | 71.673 | 1.00 | 72.05 | N | |
| ATOM | 5032 | CA | LEU | B | 284 | 36.526 | 19.531 | 71.208 | 1.00 | 71.23 | C | |
| ATOM | 5034 | CB | LEU | B | 284 | 35.534 | 19.431 | 72.391 | 1.00 | 71.20 | C | |
| ATOM | 5037 | CG | LEU | B | 284 | 35.405 | 18.117 | 73.194 | 1.00 | 71.29 | C | |
| ATOM | 5039 | CD1 | LEU | B | 284 | 36.622 | 17.826 | 74.085 | 1.00 | 71.13 | C | |
| ATOM | 5043 | CD2 | LEU | B | 284 | 34.138 | 18.161 | 74.068 | 1.00 | 71.08 | C | |
| ATOM | 5047 | C | LEU | B | 284 | 36.015 | 20.559 | 70.154 | 1.00 | 71.00 | C | |
| ATOM | 5048 | O | LEU | B | 284 | 35.242 | 20.171 | 69.280 | 1.00 | 71.12 | O | |
| ATOM | 5050 | N | LYS | B | 285 | 36.415 | 21.841 | 70.235 | 1.00 | 70.68 | N | |
| ATOM | 5051 | CA | LYS | B | 285 | 36.076 | 22.839 | 69.194 | 1.00 | 70.24 | C | |
| ATOM | 5053 | CB | LYS | B | 285 | 36.551 | 24.255 | 69.535 | 1.00 | 70.03 | C | |
| ATOM | 5056 | CG | LYS | B | 285 | 35.880 | 24.872 | 70.748 | 1.00 | 70.27 | C | |
| ATOM | 5059 | CD | LYS | B | 285 | 36.117 | 26.402 | 70.884 | 1.00 | 69.97 | C | |
| ATOM | 5062 | CE | LYS | B | 285 | 35.119 | 27.245 | 70.056 | 1.00 | 69.55 | C | |
| ATOM | 5065 | NZ | LYS | B | 285 | 35.227 | 28.732 | 70.307 | 1.00 | 69.51 | N | |
| ATOM | 5069 | C | LYS | B | 285 | 36.714 | 22.378 | 67.879 | 1.00 | 70.17 | C | |
| ATOM | 5070 | O | LYS | B | 285 | 35.993 | 22.133 | 66.886 | 1.00 | 70.44 | O | |
| ATOM | 5072 | N | GLU | B | 286 | 38.041 | 22.192 | 67.918 | 1.00 | 69.15 | N | |
| ATOM | 5073 | CA | GLU | B | 286 | 38.830 | 21.672 | 66.799 | 1.00 | 68.54 | C | |
| ATOM | 5075 | CB | GLU | B | 286 | 40.273 | 21.413 | 67.255 | 1.00 | 68.67 | C | |
| ATOM | 5078 | CG | GLU | B | 286 | 41.242 | 20.889 | 66.203 | 1.00 | 68.83 | C | |
| ATOM | 5081 | CD | GLU | B | 286 | 42.683 | 20.788 | 66.696 | 1.00 | 69.05 | C | |
| ATOM | 5082 | OE1 | GLU | B | 286 | 42.968 | 20.966 | 67.912 | 1.00 | 70.57 | O | |
| ATOM | 5083 | OE2 | GLU | B | 286 | 43.563 | 20.519 | 65.854 | 1.00 | 69.97 | O | |
| ATOM | 5084 | C | GLU | B | 286 | 38.221 | 20.404 | 66.167 | 1.00 | 68.23 | C | |
| ATOM | 5085 | O | GLU | B | 286 | 37.965 | 20.381 | 64.975 | 1.00 | 68.32 | O | |
| ATOM | 5087 | N | ALA | B | 287 | 37.948 | 19.378 | 66.963 | 1.00 | 67.62 | N | |
| ATOM | 5088 | CA | ALA | B | 287 | 37.365 | 18.125 | 66.423 | 1.00 | 67.12 | C | |
| ATOM | 5090 | CB | ALA | B | 287 | 37.233 | 17.064 | 67.512 | 1.00 | 66.71 | C | |
| ATOM | 5094 | C | ALA | B | 287 | 36.018 | 18.361 | 65.724 | 1.00 | 66.31 | C | |
| ATOM | 5095 | O | ALA | B | 287 | 35.804 | 17.901 | 64.613 | 1.00 | 66.49 | O | |
| ATOM | 5097 | N | ALA | B | 288 | 35.143 | 19.124 | 66.358 | 1.00 | 65.71 | N | |
| ATOM | 5098 | CA | ALA | B | 288 | 33.847 | 19.497 | 65.763 | 1.00 | 65.18 | C | |
| ATOM | 5100 | CB | ALA | B | 288 | 33.025 | 20.351 | 66.759 | 1.00 | 65.49 | C | |
| ATOM | 5104 | C | ALA | B | 288 | 33.970 | 20.242 | 64.431 | 1.00 | 64.17 | C | |
| ATOM | 5105 | O | ALA | B | 288 | 33.212 | 19.960 | 63.495 | 1.00 | 63.27 | O | |
| ATOM | 5107 | N | VAL | B | 289 | 34.908 | 21.196 | 64.385 | 1.00 | 63.11 | N | |
| ATOM | 5108 | CA | VAL | B | 289 | 35.193 | 21.987 | 63.182 | 1.00 | 62.82 | C | |
| ATOM | 5110 | CB | VAL | B | 289 | 36.317 | 23.069 | 63.430 | 1.00 | 63.30 | C | |
| ATOM | 5112 | CG1 | VAL | B | 289 | 36.849 | 23.716 | 62.117 | 1.00 | 63.73 | C | |
| ATOM | 5116 | CG2 | VAL | B | 289 | 35.806 | 24.187 | 64.358 | 1.00 | 62.91 | C | |
| ATOM | 5120 | C | VAL | B | 289 | 35.576 | 21.096 | 62.026 | 1.00 | 62.35 | C | |
| ATOM | 5121 | O | VAL | B | 289 | 35.101 | 21.297 | 60.927 | 1.00 | 61.52 | O | |
| ATOM | 5123 | N | MET | B | 290 | 36.415 | 20.102 | 62.321 | 1.00 | 62.83 | N | |
| ATOM | 5124 | CA | MET | B | 290 | 36.942 | 19.129 | 61.349 | 1.00 | 62.80 | C | |
| ATOM | 5126 | CB | MET | B | 290 | 38.136 | 18.372 | 61.947 | 1.00 | 62.65 | C | |
| ATOM | 5129 | CG | MET | B | 290 | 39.351 | 19.253 | 62.368 | 1.00 | 63.93 | C | |
| ATOM | 5132 | SD | MET | B | 290 | 40.994 | 18.621 | 61.814 | 1.00 | 68.60 | S | |
| ATOM | 5133 | CE | MET | B | 290 | 41.333 | 17.490 | 62.871 | 1.00 | 68.54 | C | |
| ATOM | 5137 | C | MET | B | 290 | 35.906 | 18.131 | 60.840 | 1.00 | 61.96 | C | |
| ATOM | 5138 | O | MET | B | 290 | 36.104 | 17.547 | 59.760 | 1.00 | 62.57 | O | |
| ATOM | 5140 | N | LYS | B | 291 | 34.834 | 17.908 | 61.616 | 1.00 | 61.17 | N | |
| ATOM | 5141 | CA | LYS | B | 291 | 33.659 | 17.115 | 61.175 | 1.00 | 60.27 | C | |
| ATOM | 5143 | CB | LYS | B | 291 | 32.762 | 16.722 | 62.351 | 1.00 | 59.69 | C | |
| ATOM | 5146 | CG | LYS | B | 291 | 33.405 | 15.873 | 63.427 | 1.00 | 59.63 | C | |
| ATOM | 5149 | CD | LYS | B | 291 | 32.538 | 15.794 | 64.699 | 1.00 | 60.23 | C | |
| ATOM | 5152 | CE | LYS | B | 291 | 33.222 | 14.982 | 65.824 | 1.00 | 60.65 | C | |
| ATOM | 5155 | NZ | LYS | B | 291 | 32.474 | 15.027 | 67.119 | 1.00 | 59.38 | N | |
| ATOM | 5159 | C | LYS | B | 291 | 32.819 | 17.874 | 60.151 | 1.00 | 59.66 | C | |
| ATOM | 5160 | O | LYS | B | 291 | 32.310 | 17.266 | 59.238 | 1.00 | 59.59 | O | |
| ATOM | 5162 | N | GLU | B | 292 | 32.696 | 19.197 | 60.299 | 1.00 | 59.75 | N | |
| ATOM | 5163 | CA | GLU | B | 292 | 31.893 | 20.058 | 59.407 | 1.00 | 60.03 | C | |
| ATOM | 5165 | CB | GLU | B | 292 | 31.372 | 21.292 | 60.201 | 1.00 | 60.78 | C | |
| ATOM | 5168 | CG | GLU | B | 292 | 30.267 | 20.978 | 61.214 | 1.00 | 63.30 | C | |
| ATOM | 5171 | CD | GLU | B | 292 | 29.051 | 20.319 | 60.559 | 1.00 | 66.86 | C | |
| ATOM | 5172 | OE1 | GLU | B | 292 | 28.521 | 20.912 | 59.574 | 1.00 | 68.26 | O | |
| ATOM | 5173 | OE2 | GLU | B | 292 | 28.659 | 19.199 | 60.994 | 1.00 | 67.37 | O | |
| ATOM | 5174 | C | GLU | B | 292 | 32.565 | 20.547 | 58.115 | 1.00 | 59.79 | C | |
| ATOM | 5175 | O | GLU | B | 292 | 31.937 | 21.314 | 57.348 | 1.00 | 60.02 | O | |
| ATOM | 5177 | N | ILE | B | 293 | 33.817 | 20.140 | 57.870 | 1.00 | 59.28 | N | |
| ATOM | 5178 | CA | ILE | B | 293 | 34.606 | 20.641 | 56.731 | 1.00 | 58.88 | C | |
| ATOM | 5180 | CB | ILE | B | 293 | 35.615 | 21.691 | 57.150 | 1.00 | 58.86 | C | |
| ATOM | 5182 | CG1 | ILE | B | 293 | 36.639 | 21.136 | 58.121 | 1.00 | 58.71 | C | |
| ATOM | 5185 | CD1 | ILE | B | 293 | 37.583 | 22.215 | 58.633 | 1.00 | 59.08 | C | |
| ATOM | 5189 | CG2 | ILE | B | 293 | 34.871 | 22.933 | 57.710 | 1.00 | 60.28 | C | |
| ATOM | 5193 | C | ILE | B | 293 | 35.293 | 19.552 | 55.939 | 1.00 | 57.99 | C | |
| ATOM | 5194 | O | ILE | B | 293 | 35.839 | 18.596 | 56.511 | 1.00 | 57.96 | O | |
| ATOM | 5196 | N | LYS | B | 294 | 35.223 | 19.723 | 54.613 | 1.00 | 56.92 | N | |
| ATOM | 5197 | CA | LYS | B | 294 | 35.668 | 18.738 | 53.625 | 1.00 | 56.22 | C | |
| ATOM | 5199 | CB | LYS | B | 294 | 34.612 | 17.629 | 53.439 | 1.00 | 56.16 | C | |
| ATOM | 5202 | CG | LYS | B | 294 | 34.927 | 16.475 | 52.462 | 1.00 | 55.99 | C | |
| ATOM | 5205 | CD | LYS | B | 294 | 34.259 | 15.139 | 52.974 | 1.00 | 57.03 | C | |
| ATOM | 5208 | CE | LYS | B | 294 | 34.386 | 13.885 | 52.043 | 1.00 | 56.31 | C | |
| ATOM | 5211 | NZ | LYS | B | 294 | 33.211 | 13.792 | 51.129 | 1.00 | 56.01 | N | |
| ATOM | 5215 | C | LYS | B | 294 | 35.967 | 19.542 | 52.337 | 1.00 | 54.90 | C | |
| ATOM | 5216 | O | LYS | B | 294 | 35.079 | 20.130 | 51.702 | 1.00 | 53.43 | O | |
| ATOM | 5218 | N | HIS | B | 295 | 37.262 | 19.604 | 52.039 | 1.00 | 54.29 | N | |
| ATOM | 5219 | CA | HIS | B | 295 | 37.804 | 20.192 | 50.821 | 1.00 | 53.92 | C | |
| ATOM | 5221 | CB | HIS | B | 295 | 38.096 | 21.644 | 51.096 | 1.00 | 53.38 | C | |
| ATOM | 5224 | CG | HIS | B | 295 | 38.482 | 22.394 | 49.883 | 1.00 | 53.43 | C | |
| ATOM | 5225 | ND1 | HIS | B | 295 | 39.783 | 22.447 | 49.438 | 1.00 | 52.74 | N | |
| ATOM | 5227 | CE1 | HIS | B | 295 | 39.830 | 23.161 | 48.332 | 1.00 | 54.00 | C | |
| ATOM | 5229 | NE2 | HIS | B | 295 | 38.604 | 23.574 | 48.048 | 1.00 | 54.59 | N | |
| ATOM | 5231 | CD2 | HIS | B | 295 | 37.739 | 23.091 | 48.993 | 1.00 | 53.24 | C | |
| ATOM | 5233 | C | HIS | B | 295 | 39.105 | 19.432 | 50.431 | 1.00 | 53.86 | C | |
| ATOM | 5234 | O | HIS | B | 295 | 39.801 | 18.952 | 51.332 | 1.00 | 53.84 | O | |
| ATOM | 5236 | N | PRO | B | 296 | 39.467 | 19.319 | 49.144 | 1.00 | 53.90 | N | |
| ATOM | 5237 | CA | PRO | B | 296 | 40.706 | 18.590 | 48.771 | 1.00 | 53.93 | C | |
| ATOM | 5239 | CB | PRO | B | 296 | 40.670 | 18.604 | 47.230 | 1.00 | 53.67 | C | |
| ATOM | 5242 | CG | PRO | B | 296 | 39.307 | 18.852 | 46.896 | 1.00 | 53.33 | C | |
| ATOM | 5245 | CD | PRO | B | 296 | 38.776 | 19.798 | 47.928 | 1.00 | 53.79 | C | |
| ATOM | 5248 | C | PRO | B | 296 | 42.027 | 19.177 | 49.221 | 1.00 | 53.85 | C | |
| ATOM | 5249 | O | PRO | B | 296 | 43.019 | 18.576 | 49.013 | 1.00 | 53.95 | O | |
| ATOM | 5250 | N | ASN | B | 297 | 42.020 | 20.365 | 49.788 | 1.00 | 55.34 | N | |
| ATOM | 5251 | CA | ASN | B | 297 | 43.218 | 21.103 | 50.217 | 1.00 | 56.36 | C | |
| ATOM | 5253 | CB | ASN | B | 297 | 43.438 | 22.343 | 49.329 | 1.00 | 56.22 | C | |
| ATOM | 5256 | CG | ASN | B | 297 | 43.525 | 21.988 | 47.856 | 1.00 | 56.12 | C | |
| ATOM | 5257 | OD1 | ASN | B | 297 | 42.500 | 21.876 | 47.190 | 1.00 | 55.87 | O | |
| ATOM | 5258 | ND2 | ASN | B | 297 | 44.736 | 21.800 | 47.351 | 1.00 | 55.34 | N | |
| ATOM | 5261 | C | ASN | B | 297 | 43.156 | 21.471 | 51.710 | 1.00 | 56.46 | C | |
| ATOM | 5262 | O | ASN | B | 297 | 43.820 | 22.407 | 52.137 | 1.00 | 56.41 | O | |
| ATOM | 5264 | N | LEU | B | 298 | 42.362 | 20.715 | 52.481 | 1.00 | 56.71 | N | |
| ATOM | 5265 | CA | LEU | B | 298 | 42.359 | 20.763 | 53.930 | 1.00 | 56.79 | C | |
| ATOM | 5267 | CB | LEU | B | 298 | 41.026 | 21.246 | 54.423 | 1.00 | 56.14 | C | |
| ATOM | 5270 | CG | LEU | B | 298 | 40.658 | 22.630 | 53.993 | 1.00 | 56.05 | C | |
| ATOM | 5272 | CD1 | LEU | B | 298 | 39.256 | 22.879 | 54.438 | 1.00 | 57.74 | C | |
| ATOM | 5276 | CD2 | LEU | B | 298 | 41.626 | 23.707 | 54.573 | 1.00 | 57.13 | C | |
| ATOM | 5280 | C | LEU | B | 298 | 42.586 | 19.352 | 54.414 | 1.00 | 57.67 | C | |
| ATOM | 5281 | O | LEU | B | 298 | 41.859 | 18.440 | 53.996 | 1.00 | 57.07 | O | |
| ATOM | 5283 | N | VAL | B | 299 | 43.593 | 19.169 | 55.276 | 1.00 | 59.06 | N | |
| ATOM | 5284 | CA | VAL | B | 299 | 43.886 | 17.826 | 55.869 | 1.00 | 60.00 | C | |
| ATOM | 5286 | CB | VAL | B | 299 | 45.078 | 17.814 | 56.882 | 1.00 | 59.76 | C | |
| ATOM | 5288 | CG1 | VAL | B | 299 | 44.963 | 18.914 | 57.870 | 1.00 | 64.35 | C | |
| ATOM | 5292 | CG2 | VAL | B | 299 | 45.129 | 16.539 | 57.678 | 1.00 | 59.64 | C | |
| ATOM | 5296 | C | VAL | B | 299 | 42.602 | 17.305 | 56.477 | 1.00 | 60.10 | C | |
| ATOM | 5297 | O | VAL | B | 299 | 41.877 | 18.052 | 57.106 | 1.00 | 60.50 | O | |
| ATOM | 5299 | N | GLN | B | 300 | 42.297 | 16.047 | 56.204 | 1.00 | 61.87 | N | |
| ATOM | 5300 | CA | GLN | B | 300 | 40.955 | 15.473 | 56.399 | 1.00 | 62.49 | C | |
| ATOM | 5302 | CB | GLN | B | 300 | 40.507 | 14.660 | 55.169 | 1.00 | 61.97 | C | |
| ATOM | 5305 | CG | GLN | B | 300 | 39.081 | 14.054 | 55.300 | 1.00 | 62.54 | C | |
| ATOM | 5308 | CD | GLN | B | 300 | 37.958 | 15.033 | 55.030 | 1.00 | 63.67 | C | |
| ATOM | 5309 | OE1 | GLN | B | 300 | 37.810 | 15.493 | 53.892 | 1.00 | 67.89 | O | |
| ATOM | 5310 | NE2 | GLN | B | 300 | 37.122 | 15.301 | 56.038 | 1.00 | 60.56 | N | |
| ATOM | 5313 | C | GLN | B | 300 | 40.889 | 14.629 | 57.676 | 1.00 | 63.05 | C | |
| ATOM | 5314 | O | GLN | B | 300 | 41.739 | 13.773 | 57.912 | 1.00 | 61.84 | O | |
| ATOM | 5316 | N | LEU | B | 301 | 39.833 | 14.885 | 58.453 | 1.00 | 64.73 | N | |
| ATOM | 5317 | CA | LEU | B | 301 | 39.482 | 14.089 | 59.604 | 1.00 | 65.54 | C | |
| ATOM | 5319 | CB | LEU | B | 301 | 38.363 | 14.768 | 60.361 | 1.00 | 65.78 | C | |
| ATOM | 5322 | CG | LEU | B | 301 | 37.798 | 14.077 | 61.611 | 1.00 | 67.12 | C | |
| ATOM | 5324 | CD1 | LEU | B | 301 | 38.899 | 13.861 | 62.670 | 1.00 | 69.43 | C | |
| ATOM | 5328 | CD2 | LEU | B | 301 | 36.615 | 14.917 | 62.183 | 1.00 | 67.01 | C | |
| ATOM | 5332 | C | LEU | B | 301 | 39.009 | 12.718 | 59.146 | 1.00 | 66.39 | C | |
| ATOM | 5333 | O | LEU | B | 301 | 38.188 | 12.610 | 58.231 | 1.00 | 66.26 | O | |
| ATOM | 5335 | N | LEU | B | 302 | 39.557 | 11.691 | 59.786 | 1.00 | 67.35 | N | |
| ATOM | 5336 | CA | LEU | B | 302 | 39.168 | 10.303 | 59.597 | 1.00 | 68.11 | C | |
| ATOM | 5338 | CB | LEU | B | 302 | 40.405 | 9.477 | 59.260 | 1.00 | 68.69 | C | |
| ATOM | 5341 | CG | LEU | B | 302 | 41.315 | 9.944 | 58.133 | 1.00 | 69.48 | C | |
| ATOM | 5343 | CD1 | LEU | B | 302 | 42.592 | 9.098 | 58.153 | 1.00 | 69.38 | C | |
| ATOM | 5347 | CD2 | LEU | B | 302 | 40.570 | 9.817 | 56.786 | 1.00 | 70.13 | C | |
| ATOM | 5351 | C | LEU | B | 302 | 38.533 | 9.658 | 60.828 | 1.00 | 68.40 | C | |
| ATOM | 5352 | O | LEU | B | 302 | 37.843 | 8.650 | 60.694 | 1.00 | 68.99 | O | |
| ATOM | 5354 | N | GLY | B | 303 | 38.817 | 10.160 | 62.021 | 1.00 | 68.75 | N | |
| ATOM | 5355 | CA | GLY | B | 303 | 38.275 | 9.549 | 63.231 | 1.00 | 69.49 | C | |
| ATOM | 5358 | C | GLY | B | 303 | 38.666 | 10.295 | 64.476 | 1.00 | 70.27 | C | |
| ATOM | 5359 | O | GLY | B | 303 | 39.460 | 11.249 | 64.410 | 1.00 | 70.16 | O | |
| ATOM | 5361 | N | VAL | B | 304 | 38.072 | 9.890 | 65.596 | 1.00 | 71.26 | N | |
| ATOM | 5362 | CA | VAL | B | 304 | 38.339 | 10.511 | 66.914 | 1.00 | 72.27 | C | |
| ATOM | 5364 | CB | VAL | B | 304 | 37.415 | 11.765 | 67.239 | 1.00 | 72.22 | C | |
| ATOM | 5366 | CG1 | VAL | B | 304 | 37.713 | 12.972 | 66.328 | 1.00 | 71.24 | C | |
| ATOM | 5370 | CG2 | VAL | B | 304 | 35.913 | 11.397 | 67.209 | 1.00 | 71.95 | C | |
| ATOM | 5374 | C | VAL | B | 304 | 38.147 | 9.512 | 68.034 | 1.00 | 73.39 | C | |
| ATOM | 5375 | O | VAL | B | 304 | 37.552 | 8.443 | 67.827 | 1.00 | 73.23 | O | |
| ATOM | 5377 | N | CYS | B | 305 | 38.653 | 9.877 | 69.213 | 1.00 | 75.11 | N | |
| ATOM | 5378 | CA | CYS | B | 305 | 38.310 | 9.202 | 70.487 | 1.00 | 76.71 | C | |
| ATOM | 5380 | CB | CYS | B | 305 | 39.469 | 8.304 | 70.923 | 1.00 | 76.71 | C | |
| ATOM | 5383 | SG | CYS | B | 305 | 40.061 | 7.284 | 69.567 | 1.00 | 80.06 | S | |
| ATOM | 5385 | C | CYS | B | 305 | 37.991 | 10.242 | 71.563 | 1.00 | 76.81 | C | |
| ATOM | 5386 | O | CYS | B | 305 | 38.886 | 10.647 | 72.298 | 1.00 | 76.76 | O | |
| ATOM | 5388 | N | THR | B | 306 | 36.729 | 10.686 | 71.625 | 1.00 | 77.27 | N | |
| ATOM | 5389 | CA | THR | B | 306 | 36.302 | 11.767 | 72.552 | 1.00 | 77.61 | C | |
| ATOM | 5391 | CB | THR | B | 306 | 35.975 | 13.086 | 71.769 | 1.00 | 77.86 | C | |
| ATOM | 5393 | OG1 | THR | B | 306 | 34.943 | 12.872 | 70.795 | 1.00 | 77.79 | O | |
| ATOM | 5395 | CG2 | THR | B | 306 | 37.209 | 13.576 | 70.950 | 1.00 | 77.27 | C | |
| ATOM | 5399 | C | THR | B | 306 | 35.195 | 11.350 | 73.553 | 1.00 | 78.19 | C | |
| ATOM | 5400 | O | THR | B | 306 | 34.187 | 12.049 | 73.742 | 1.00 | 78.09 | O | |
| ATOM | 5402 | N | ARG | B | 307 | 35.434 | 10.193 | 74.185 | 1.00 | 78.84 | N | |
| ATOM | 5403 | CA | ARG | B | 307 | 34.690 | 9.699 | 75.354 | 1.00 | 79.28 | C | |
| ATOM | 5405 | CB | ARG | B | 307 | 33.957 | 8.374 | 75.040 | 1.00 | 79.16 | C | |
| ATOM | 5408 | CG | ARG | B | 307 | 32.560 | 8.541 | 74.401 | 1.00 | 79.65 | C | |
| ATOM | 5411 | CD | ARG | B | 307 | 32.486 | 8.360 | 72.864 | 1.00 | 79.25 | C | |
| ATOM | 5414 | NE | ARG | B | 307 | 31.243 | 8.928 | 72.288 | 1.00 | 78.87 | N | |
| ATOM | 5416 | CZ | ARG | B | 307 | 30.468 | 8.387 | 71.319 | 1.00 | 77.88 | C | |
| ATOM | 5417 | NH1 | ARG | B | 307 | 30.743 | 7.199 | 70.748 | 1.00 | 77.45 | N | |
| ATOM | 5420 | NH2 | ARG | B | 307 | 29.382 | 9.058 | 70.917 | 1.00 | 77.41 | N | |
| ATOM | 5423 | C | ARG | B | 307 | 35.683 | 9.529 | 76.513 | 1.00 | 79.49 | C | |
| ATOM | 5424 | O | ARG | B | 307 | 35.660 | 10.323 | 77.465 | 1.00 | 79.71 | O | |
| ATOM | 5426 | N | GLU | B | 308 | 36.579 | 8.540 | 76.393 | 1.00 | 79.48 | N | |
| ATOM | 5427 | CA | GLU | B | 308 | 37.615 | 8.276 | 77.411 | 1.00 | 79.30 | C | |
| ATOM | 5429 | CB | GLU | B | 308 | 38.102 | 6.807 | 77.455 | 1.00 | 79.29 | C | |
| ATOM | 5432 | CG | GLU | B | 308 | 37.172 | 5.716 | 76.913 | 1.00 | 79.56 | C | |
| ATOM | 5435 | CD | GLU | B | 308 | 37.372 | 5.446 | 75.423 | 1.00 | 79.93 | C | |
| ATOM | 5436 | OE1 | GLU | B | 308 | 37.704 | 6.396 | 74.674 | 1.00 | 80.96 | O | |
| ATOM | 5437 | OE2 | GLU | B | 308 | 37.198 | 4.291 | 74.994 | 1.00 | 79.29 | O | |
| ATOM | 5438 | C | GLU | B | 308 | 38.805 | 9.194 | 77.061 | 1.00 | 79.29 | C | |
| ATOM | 5439 | O | GLU | B | 308 | 39.079 | 9.383 | 75.869 | 1.00 | 79.13 | O | |
| ATOM | 5441 | N | PRO | B | 309 | 39.486 | 9.782 | 78.058 | 1.00 | 79.04 | N | |
| ATOM | 5442 | CA | PRO | B | 309 | 40.865 | 10.284 | 77.868 | 1.00 | 78.98 | C | |
| ATOM | 5444 | CB | PRO | B | 309 | 41.143 | 11.049 | 79.174 | 1.00 | 78.80 | C | |
| ATOM | 5447 | CG | PRO | B | 309 | 39.823 | 11.298 | 79.759 | 1.00 | 78.63 | C | |
| ATOM | 5450 | CD | PRO | B | 309 | 38.989 | 10.114 | 79.403 | 1.00 | 78.87 | C | |
| ATOM | 5453 | C | PRO | B | 309 | 41.898 | 9.149 | 77.675 | 1.00 | 78.88 | C | |
| ATOM | 5454 | O | PRO | B | 309 | 41.570 | 7.995 | 77.959 | 1.00 | 78.52 | O | |
| ATOM | 5455 | N | PRO | B | 310 | 43.137 | 9.450 | 77.258 | 1.00 | 78.99 | N | |
| ATOM | 5456 | CA | PRO | B | 310 | 43.547 | 10.648 | 76.466 | 1.00 | 79.02 | C | |
| ATOM | 5458 | CB | PRO | B | 310 | 45.081 | 10.508 | 76.412 | 1.00 | 79.17 | C | |
| ATOM | 5461 | CG | PRO | B | 310 | 45.358 | 9.153 | 77.021 | 1.00 | 79.45 | C | |
| ATOM | 5464 | CD | PRO | B | 310 | 44.271 | 8.926 | 78.033 | 1.00 | 79.06 | C | |
| ATOM | 5467 | C | PRO | B | 310 | 43.008 | 10.798 | 75.027 | 1.00 | 78.50 | C | |
| ATOM | 5468 | O | PRO | B | 310 | 43.185 | 9.906 | 74.193 | 1.00 | 78.68 | O | |
| ATOM | 5469 | N | PHE | B | 311 | 42.457 | 11.967 | 74.723 | 1.00 | 77.83 | N | |
| ATOM | 5470 | CA | PHE | B | 311 | 41.713 | 12.178 | 73.478 | 1.00 | 77.56 | C | |
| ATOM | 5472 | CB | PHE | B | 311 | 40.849 | 13.464 | 73.554 | 1.00 | 78.13 | C | |
| ATOM | 5475 | CG | PHE | B | 311 | 39.906 | 13.495 | 74.763 | 1.00 | 78.77 | C | |
| ATOM | 5476 | CD1 | PHE | B | 311 | 39.007 | 12.439 | 74.999 | 1.00 | 79.88 | C | |
| ATOM | 5478 | CE1 | PHE | B | 311 | 38.142 | 12.430 | 76.120 | 1.00 | 79.52 | C | |
| ATOM | 5480 | CZ | PHE | B | 311 | 38.180 | 13.483 | 77.031 | 1.00 | 79.10 | C | |
| ATOM | 5482 | CE2 | PHE | B | 311 | 39.083 | 14.538 | 76.820 | 1.00 | 78.86 | C | |
| ATOM | 5484 | CD2 | PHE | B | 311 | 39.944 | 14.542 | 75.689 | 1.00 | 78.70 | C | |
| ATOM | 5486 | C | PHE | B | 311 | 42.619 | 12.144 | 72.220 | 1.00 | 76.93 | C | |
| ATOM | 5487 | O | PHE | B | 311 | 43.850 | 12.305 | 72.298 | 1.00 | 77.27 | O | |
| ATOM | 5489 | N | TYR | B | 312 | 41.988 | 11.877 | 71.078 | 1.00 | 75.60 | N | |
| ATOM | 5490 | CA | TYR | B | 312 | 42.671 | 11.619 | 69.810 | 1.00 | 74.16 | C | |
| ATOM | 5492 | CB | TYR | B | 312 | 42.716 | 10.106 | 69.500 | 1.00 | 74.48 | C | |
| ATOM | 5495 | CG | TYR | B | 312 | 43.797 | 9.223 | 70.111 | 1.00 | 74.54 | C | |
| ATOM | 5496 | CD1 | TYR | B | 312 | 44.983 | 9.726 | 70.698 | 1.00 | 74.24 | C | |
| ATOM | 5498 | CE1 | TYR | B | 312 | 45.946 | 8.839 | 71.221 | 1.00 | 74.30 | C | |
| ATOM | 5500 | CZ | TYR | B | 312 | 45.713 | 7.452 | 71.129 | 1.00 | 74.47 | C | |
| ATOM | 5501 | OH | TYR | B | 312 | 46.575 | 6.503 | 71.609 | 1.00 | 74.29 | O | |
| ATOM | 5503 | CE2 | TYR | B | 312 | 44.570 | 6.971 | 70.553 | 1.00 | 73.85 | C | |
| ATOM | 5505 | CD2 | TYR | B | 312 | 43.640 | 7.841 | 70.044 | 1.00 | 73.73 | C | |
| ATOM | 5507 | C | TYR | B | 312 | 41.869 | 12.238 | 68.680 | 1.00 | 72.73 | C | |
| ATOM | 5508 | O | TYR | B | 312 | 40.640 | 12.100 | 68.647 | 1.00 | 72.52 | O | |
| ATOM | 5510 | N | ILE | B | 313 | 42.580 | 12.868 | 67.750 | 1.00 | 70.96 | N | |
| ATOM | 5511 | CA | ILE | B | 313 | 42.081 | 13.184 | 66.422 | 1.00 | 69.57 | C | |
| ATOM | 5513 | CB | ILE | B | 313 | 42.190 | 14.692 | 66.183 | 1.00 | 69.43 | C | |
| ATOM | 5515 | CG1 | ILE | B | 313 | 41.189 | 15.478 | 67.065 | 1.00 | 68.90 | C | |
| ATOM | 5518 | CD1 | ILE | B | 313 | 41.571 | 16.948 | 67.330 | 1.00 | 68.29 | C | |
| ATOM | 5522 | CG2 | ILE | B | 313 | 41.978 | 14.999 | 64.721 | 1.00 | 68.84 | C | |
| ATOM | 5526 | C | ILE | B | 313 | 42.954 | 12.445 | 65.390 | 1.00 | 68.99 | C | |
| ATOM | 5527 | O | ILE | B | 313 | 44.139 | 12.741 | 65.273 | 1.00 | 69.34 | O | |
| ATOM | 5529 | N | ILE | B | 314 | 42.360 | 11.548 | 64.613 | 1.00 | 67.46 | N | |
| ATOM | 5530 | CA | ILE | B | 314 | 43.078 | 10.837 | 63.569 | 1.00 | 67.04 | C | |
| ATOM | 5532 | CB | ILE | B | 314 | 42.600 | 9.337 | 63.471 | 1.00 | 67.19 | C | |
| ATOM | 5534 | CG1 | ILE | B | 314 | 43.132 | 8.495 | 64.633 | 1.00 | 67.44 | C | |
| ATOM | 5537 | CD1 | ILE | B | 314 | 42.847 | 9.003 | 65.998 | 1.00 | 67.98 | C | |
| ATOM | 5541 | CG2 | ILE | B | 314 | 43.120 | 8.637 | 62.198 | 1.00 | 66.89 | C | |
| ATOM | 5545 | C | ILE | B | 314 | 42.795 | 11.572 | 62.283 | 1.00 | 66.44 | C | |
| ATOM | 5546 | O | ILE | B | 314 | 41.642 | 11.656 | 61.898 | 1.00 | 66.69 | O | |
| ATOM | 5548 | N | THR | B | 315 | 43.826 | 12.078 | 61.608 | 1.00 | 66.12 | N | |
| ATOM | 5549 | CA | THR | B | 315 | 43.684 | 12.584 | 60.227 | 1.00 | 66.33 | C | |
| ATOM | 5551 | CB | THR | B | 315 | 44.067 | 14.093 | 60.139 | 1.00 | 65.90 | C | |
| ATOM | 5553 | OG1 | THR | B | 315 | 45.466 | 14.232 | 60.300 | 1.00 | 67.05 | O | |
| ATOM | 5555 | CG2 | THR | B | 315 | 43.512 | 14.924 | 61.295 | 1.00 | 66.14 | C | |
| ATOM | 5559 | C | THR | B | 315 | 44.516 | 11.764 | 59.200 | 1.00 | 66.52 | C | |
| ATOM | 5560 | O | THR | B | 315 | 45.261 | 10.845 | 59.553 | 1.00 | 66.67 | O | |
| ATOM | 5562 | N | GLU | B | 316 | 44.391 | 12.119 | 57.923 | 1.00 | 66.70 | N | |
| ATOM | 5563 | CA | GLU | B | 316 | 45.316 | 11.623 | 56.887 | 1.00 | 66.85 | C | |
| ATOM | 5565 | CB | GLU | B | 316 | 44.898 | 12.091 | 55.471 | 1.00 | 67.10 | C | |
| ATOM | 5568 | CG | GLU | B | 316 | 44.974 | 13.593 | 55.192 | 1.00 | 67.03 | C | |
| ATOM | 5571 | CD | GLU | B | 316 | 44.267 | 13.993 | 53.884 | 1.00 | 68.03 | C | |
| ATOM | 5572 | OE1 | GLU | B | 316 | 44.448 | 13.319 | 52.841 | 1.00 | 66.56 | O | |
| ATOM | 5573 | OE2 | GLU | B | 316 | 43.523 | 15.003 | 53.879 | 1.00 | 72.06 | O | |
| ATOM | 5574 | C | GLU | B | 316 | 46.760 | 12.061 | 57.177 | 1.00 | 66.17 | C | |
| ATOM | 5575 | O | GLU | B | 316 | 46.978 | 13.125 | 57.759 | 1.00 | 66.38 | O | |
| ATOM | 5577 | N | PHE | B | 317 | 47.709 | 11.239 | 56.743 | 1.00 | 65.06 | N | |
| ATOM | 5578 | CA | PHE | B | 317 | 49.127 | 11.482 | 56.905 | 1.00 | 64.62 | C | |
| ATOM | 5580 | CB | PHE | B | 317 | 49.872 | 10.176 | 57.245 | 1.00 | 65.17 | C | |
| ATOM | 5583 | CG | PHE | B | 317 | 51.356 | 10.356 | 57.383 | 1.00 | 66.01 | C | |
| ATOM | 5584 | CD1 | PHE | B | 317 | 51.876 | 11.177 | 58.388 | 1.00 | 67.15 | C | |
| ATOM | 5586 | CE1 | PHE | B | 317 | 53.259 | 11.380 | 58.497 | 1.00 | 67.42 | C | |
| ATOM | 5588 | CZ | PHE | B | 317 | 54.125 | 10.773 | 57.587 | 1.00 | 67.18 | C | |
| ATOM | 5590 | CE2 | PHE | B | 317 | 53.610 | 9.963 | 56.573 | 1.00 | 66.79 | C | |
| ATOM | 5592 | CD2 | PHE | B | 317 | 52.234 | 9.775 | 56.466 | 1.00 | 66.42 | C | |
| ATOM | 5594 | C | PHE | B | 317 | 49.673 | 12.074 | 55.608 | 1.00 | 63.60 | C | |
| ATOM | 5595 | O | PHE | B | 317 | 49.302 | 11.640 | 54.523 | 1.00 | 63.73 | O | |
| ATOM | 5597 | N | MET | B | 318 | 50.605 | 13.010 | 55.741 | 1.00 | 62.40 | N | |
| ATOM | 5598 | CA | MET | B | 318 | 51.143 | 13.760 | 54.636 | 1.00 | 61.97 | C | |
| ATOM | 5600 | CB | MET | B | 318 | 51.043 | 15.250 | 54.937 | 1.00 | 62.09 | C | |
| ATOM | 5603 | CG | MET | B | 318 | 49.586 | 15.701 | 55.031 | 1.00 | 62.79 | C | |
| ATOM | 5606 | SD | MET | B | 318 | 48.566 | 15.458 | 53.539 | 1.00 | 65.59 | S | |
| ATOM | 5607 | CE | MET | B | 318 | 49.495 | 16.137 | 52.238 | 1.00 | 65.71 | C | |
| ATOM | 5611 | C | MET | B | 318 | 52.576 | 13.309 | 54.380 | 1.00 | 61.63 | C | |
| ATOM | 5612 | O | MET | B | 318 | 53.450 | 13.382 | 55.259 | 1.00 | 62.01 | O | |
| ATOM | 5614 | N | THR | B | 319 | 52.800 | 12.860 | 53.144 | 1.00 | 60.85 | N | |
| ATOM | 5615 | CA | THR | B | 319 | 53.886 | 11.929 | 52.803 | 1.00 | 59.80 | C | |
| ATOM | 5617 | CB | THR | B | 319 | 53.416 | 11.105 | 51.607 | 1.00 | 59.64 | C | |
| ATOM | 5619 | OG1 | THR | B | 319 | 52.476 | 10.139 | 52.082 | 1.00 | 58.47 | O | |
| ATOM | 5621 | CG2 | THR | B | 319 | 54.476 | 10.241 | 51.024 | 1.00 | 61.13 | C | |
| ATOM | 5625 | C | THR | B | 319 | 55.212 | 12.633 | 52.554 | 1.00 | 59.25 | C | |
| ATOM | 5626 | O | THR | B | 319 | 56.258 | 12.011 | 52.679 | 1.00 | 59.56 | O | |
| ATOM | 5628 | N | TYR | B | 320 | 55.150 | 13.929 | 52.232 | 1.00 | 58.42 | N | |
| ATOM | 5629 | CA | TYR | B | 320 | 56.288 | 14.741 | 51.866 | 1.00 | 56.99 | C | |
| ATOM | 5631 | CB | TYR | B | 320 | 56.071 | 15.244 | 50.428 | 1.00 | 57.33 | C | |
| ATOM | 5634 | CG | TYR | B | 320 | 56.125 | 14.111 | 49.371 | 1.00 | 57.70 | C | |
| ATOM | 5635 | CD1 | TYR | B | 320 | 55.032 | 13.267 | 49.158 | 1.00 | 58.13 | C | |
| ATOM | 5637 | CE1 | TYR | B | 320 | 55.074 | 12.230 | 48.192 | 1.00 | 58.84 | C | |
| ATOM | 5639 | CZ | TYR | B | 320 | 56.231 | 12.023 | 47.418 | 1.00 | 58.72 | C | |
| ATOM | 5640 | OH | TYR | B | 320 | 56.268 | 10.955 | 46.480 | 1.00 | 57.02 | O | |
| ATOM | 5642 | CE2 | TYR | B | 320 | 57.330 | 12.878 | 47.608 | 1.00 | 57.30 | C | |
| ATOM | 5644 | CD2 | TYR | B | 320 | 57.276 | 13.899 | 48.577 | 1.00 | 57.97 | C | |
| ATOM | 5646 | C | TYR | B | 320 | 56.585 | 15.883 | 52.881 | 1.00 | 56.79 | C | |
| ATOM | 5647 | O | TYR | B | 320 | 57.490 | 16.690 | 52.630 | 1.00 | 57.93 | O | |
| ATOM | 5649 | N | GLY | B | 321 | 55.894 | 15.950 | 54.027 | 1.00 | 55.52 | N | |
| ATOM | 5650 | CA | GLY | B | 321 | 56.308 | 16.828 | 55.126 | 1.00 | 55.01 | C | |
| ATOM | 5653 | C | GLY | B | 321 | 55.853 | 18.255 | 54.962 | 1.00 | 54.92 | C | |
| ATOM | 5654 | O | GLY | B | 321 | 54.983 | 18.522 | 54.167 | 1.00 | 55.89 | O | |
| ATOM | 5656 | N | ASN | B | 322 | 56.392 | 19.193 | 55.715 | 1.00 | 54.81 | N | |
| ATOM | 5657 | CA | ASN | B | 322 | 55.813 | 20.531 | 55.676 | 1.00 | 55.82 | C | |
| ATOM | 5659 | CB | ASN | B | 322 | 55.934 | 21.333 | 56.986 | 1.00 | 56.67 | C | |
| ATOM | 5662 | CG | ASN | B | 322 | 57.322 | 21.772 | 57.250 | 1.00 | 59.45 | C | |
| ATOM | 5663 | OD1 | ASN | B | 322 | 58.230 | 20.945 | 57.291 | 1.00 | 60.95 | O | |
| ATOM | 5664 | ND2 | ASN | B | 322 | 57.515 | 23.073 | 57.444 | 1.00 | 64.38 | N | |
| ATOM | 5667 | C | ASN | B | 322 | 56.409 | 21.290 | 54.573 | 1.00 | 55.03 | C | |
| ATOM | 5668 | O | ASN | B | 322 | 57.461 | 20.940 | 54.090 | 1.00 | 55.55 | O | |
| ATOM | 5670 | N | LEU | B | 323 | 55.737 | 22.375 | 54.238 | 1.00 | 54.09 | N | |
| ATOM | 5671 | CA | LEU | B | 323 | 56.062 | 23.151 | 53.094 | 1.00 | 53.43 | C | |
| ATOM | 5673 | CB | LEU | B | 323 | 54.852 | 24.022 | 52.759 | 1.00 | 53.53 | C | |
| ATOM | 5676 | CG | LEU | B | 323 | 54.774 | 24.640 | 51.372 | 1.00 | 54.13 | C | |
| ATOM | 5678 | CD1 | LEU | B | 323 | 54.537 | 23.519 | 50.346 | 1.00 | 55.21 | C | |
| ATOM | 5682 | CD2 | LEU | B | 323 | 53.712 | 25.776 | 51.301 | 1.00 | 53.75 | C | |
| ATOM | 5686 | C | LEU | B | 323 | 57.364 | 23.990 | 53.260 | 1.00 | 53.45 | C | |
| ATOM | 5687 | O | LEU | B | 323 | 57.955 | 24.354 | 52.260 | 1.00 | 54.68 | O | |
| ATOM | 5689 | N | LEU | B | 324 | 57.838 | 24.290 | 54.466 | 1.00 | 52.58 | N | |
| ATOM | 5690 | CA | LEU | B | 324 | 59.178 | 24.900 | 54.606 | 1.00 | 52.25 | C | |
| ATOM | 5692 | CB | LEU | B | 324 | 59.474 | 25.354 | 56.034 | 1.00 | 52.59 | C | |
| ATOM | 5695 | CG | LEU | B | 324 | 58.666 | 26.544 | 56.508 | 1.00 | 53.69 | C | |
| ATOM | 5697 | CD1 | LEU | B | 324 | 59.289 | 27.008 | 57.822 | 1.00 | 53.57 | C | |
| ATOM | 5701 | CD2 | LEU | B | 324 | 58.671 | 27.696 | 55.448 | 1.00 | 54.16 | C | |
| ATOM | 5705 | C | LEU | B | 324 | 60.324 | 24.014 | 54.212 | 1.00 | 50.92 | C | |
| ATOM | 5706 | O | LEU | B | 324 | 61.124 | 24.372 | 53.366 | 1.00 | 50.69 | O | |
| ATOM | 5708 | N | ASP | B | 325 | 60.403 | 22.865 | 54.844 | 1.00 | 49.78 | N | |
| ATOM | 5709 | CA | ASP | B | 325 | 61.489 | 21.928 | 54.584 | 1.00 | 49.19 | C | |
| ATOM | 5711 | CB | ASP | B | 325 | 61.417 | 20.724 | 55.515 | 1.00 | 49.24 | C | |
| ATOM | 5714 | CG | ASP | B | 325 | 61.399 | 21.108 | 56.992 | 1.00 | 52.83 | C | |
| ATOM | 5715 | OD1 | ASP | B | 325 | 61.737 | 22.282 | 57.394 | 1.00 | 59.64 | O | |
| ATOM | 5716 | OD2 | ASP | B | 325 | 61.047 | 20.265 | 57.845 | 1.00 | 59.33 | O | |
| ATOM | 5717 | C | ASP | B | 325 | 61.472 | 21.465 | 53.122 | 1.00 | 47.84 | C | |
| ATOM | 5718 | O | ASP | B | 325 | 62.532 | 21.397 | 52.472 | 1.00 | 47.41 | O | |
| ATOM | 5720 | N | TYR | B | 326 | 60.277 | 21.212 | 52.603 | 1.00 | 45.92 | N | |
| ATOM | 5721 | CA | TYR | B | 326 | 60.129 | 20.872 | 51.208 | 1.00 | 45.83 | C | |
| ATOM | 5723 | CB | TYR | B | 326 | 58.652 | 20.647 | 50.891 | 1.00 | 46.16 | C | |
| ATOM | 5726 | CG | TYR | B | 326 | 58.355 | 20.096 | 49.526 | 1.00 | 45.58 | C | |
| ATOM | 5727 | CD1 | TYR | B | 326 | 58.442 | 18.737 | 49.281 | 1.00 | 46.52 | C | |
| ATOM | 5729 | CE1 | TYR | B | 326 | 58.129 | 18.213 | 48.019 | 1.00 | 46.47 | C | |
| ATOM | 5731 | CZ | TYR | B | 326 | 57.722 | 19.076 | 46.997 | 1.00 | 45.99 | C | |
| ATOM | 5732 | OH | TYR | B | 326 | 57.453 | 18.570 | 45.769 | 1.00 | 46.68 | O | |
| ATOM | 5734 | CE2 | TYR | B | 326 | 57.605 | 20.416 | 47.219 | 1.00 | 44.66 | C | |
| ATOM | 5736 | CD2 | TYR | B | 326 | 57.920 | 20.930 | 48.490 | 1.00 | 45.75 | C | |
| ATOM | 5738 | C | TYR | B | 326 | 60.712 | 21.964 | 50.294 | 1.00 | 45.56 | C | |
| ATOM | 5739 | O | TYR | B | 326 | 61.575 | 21.694 | 49.457 | 1.00 | 45.86 | O | |
| ATOM | 5741 | N | LEU | B | 327 | 60.214 | 23.182 | 50.458 | 1.00 | 44.81 | N | |
| ATOM | 5742 | CA | LEU | B | 327 | 60.671 | 24.294 | 49.695 | 1.00 | 44.18 | C | |
| ATOM | 5744 | CB | LEU | B | 327 | 59.825 | 25.512 | 50.065 | 1.00 | 44.60 | C | |
| ATOM | 5747 | CG | LEU | B | 327 | 58.433 | 25.703 | 49.440 | 1.00 | 44.78 | C | |
| ATOM | 5749 | CD1 | LEU | B | 327 | 57.741 | 26.874 | 50.137 | 1.00 | 45.05 | C | |
| ATOM | 5753 | CD2 | LEU | B | 327 | 58.490 | 25.999 | 47.889 | 1.00 | 43.39 | C | |
| ATOM | 5757 | C | LEU | B | 327 | 62.201 | 24.597 | 49.873 | 1.00 | 44.18 | C | |
| ATOM | 5758 | O | LEU | B | 327 | 62.865 | 24.960 | 48.915 | 1.00 | 43.53 | O | |
| ATOM | 5760 | N | ARG | B | 328 | 62.722 | 24.462 | 51.097 | 1.00 | 44.12 | N | |
| ATOM | 5761 | CA | ARG | B | 328 | 64.140 | 24.635 | 51.413 | 1.00 | 44.29 | C | |
| ATOM | 5763 | CB | ARG | B | 328 | 64.356 | 24.590 | 52.918 | 1.00 | 44.11 | C | |
| ATOM | 5766 | CG | ARG | B | 328 | 63.912 | 25.845 | 53.614 | 1.00 | 47.22 | C | |
| ATOM | 5769 | CD | ARG | B | 328 | 63.732 | 25.780 | 55.147 | 1.00 | 47.72 | C | |
| ATOM | 5772 | NE | ARG | B | 328 | 63.628 | 27.155 | 55.679 | 1.00 | 51.89 | N | |
| ATOM | 5774 | CZ | ARG | B | 328 | 63.153 | 27.526 | 56.888 | 1.00 | 54.30 | C | |
| ATOM | 5775 | NH1 | ARG | B | 328 | 62.702 | 26.633 | 57.767 | 1.00 | 56.91 | N | |
| ATOM | 5778 | NH2 | ARG | B | 328 | 63.115 | 28.828 | 57.222 | 1.00 | 52.31 | N | |
| ATOM | 5781 | C | ARG | B | 328 | 65.040 | 23.596 | 50.811 | 1.00 | 44.28 | C | |
| ATOM | 5782 | O | ARG | B | 328 | 66.183 | 23.872 | 50.626 | 1.00 | 44.29 | O | |
| ATOM | 5784 | N | GLU | B | 329 | 64.537 | 22.393 | 50.546 | 1.00 | 44.77 | N | |
| ATOM | 5785 | CA | GLU | B | 329 | 65.337 | 21.253 | 50.088 | 1.00 | 45.63 | C | |
| ATOM | 5787 | CB | GLU | B | 329 | 65.060 | 20.085 | 51.011 | 1.00 | 45.67 | C | |
| ATOM | 5790 | CG | GLU | B | 329 | 65.517 | 20.396 | 52.413 | 1.00 | 48.46 | C | |
| ATOM | 5793 | CD | GLU | B | 329 | 65.010 | 19.450 | 53.485 | 1.00 | 49.88 | C | |
| ATOM | 5794 | OE1 | GLU | B | 329 | 64.371 | 18.373 | 53.188 | 1.00 | 53.29 | O | |
| ATOM | 5795 | OE2 | GLU | B | 329 | 65.275 | 19.841 | 54.664 | 1.00 | 54.85 | O | |
| ATOM | 5796 | C | GLU | B | 329 | 65.113 | 20.774 | 48.649 | 1.00 | 45.25 | C | |
| ATOM | 5797 | O | GLU | B | 329 | 65.888 | 19.921 | 48.163 | 1.00 | 45.50 | O | |
| ATOM | 5799 | N | CYS | B | 330 | 64.080 | 21.304 | 47.977 | 1.00 | 44.83 | N | |
| ATOM | 5800 | CA | CYS | B | 330 | 63.706 | 20.921 | 46.607 | 1.00 | 44.55 | C | |
| ATOM | 5802 | CB | CYS | B | 330 | 62.419 | 21.641 | 46.161 | 1.00 | 43.80 | C | |
| ATOM | 5805 | SG | CYS | B | 330 | 62.531 | 23.462 | 46.036 | 1.00 | 46.00 | S | |
| ATOM | 5807 | C | CYS | B | 330 | 64.848 | 21.192 | 45.601 | 1.00 | 43.89 | C | |
| ATOM | 5808 | O | CYS | B | 330 | 65.846 | 21.878 | 45.933 | 1.00 | 44.55 | O | |
| ATOM | 5810 | N | ASN | B | 331 | 64.721 | 20.577 | 44.416 | 1.00 | 42.32 | N | |
| ATOM | 5811 | CA | ASN | B | 331 | 65.487 | 20.922 | 43.219 | 1.00 | 40.75 | C | |
| ATOM | 5813 | CB | ASN | B | 331 | 65.657 | 19.689 | 42.328 | 1.00 | 39.93 | C | |
| ATOM | 5816 | CG | ASN | B | 331 | 66.144 | 20.001 | 40.951 | 1.00 | 39.67 | C | |
| ATOM | 5817 | OD1 | ASN | B | 331 | 65.978 | 21.101 | 40.449 | 1.00 | 43.10 | O | |
| ATOM | 5818 | ND2 | ASN | B | 331 | 66.706 | 19.018 | 40.297 | 1.00 | 37.26 | N | |
| ATOM | 5821 | C | ASN | B | 331 | 64.633 | 21.993 | 42.561 | 1.00 | 40.34 | C | |
| ATOM | 5822 | O | ASN | B | 331 | 63.588 | 21.680 | 42.047 | 1.00 | 40.60 | O | |
| ATOM | 5824 | N | ARG | B | 332 | 65.090 | 23.240 | 42.536 | 1.00 | 40.06 | N | |
| ATOM | 5825 | CA | ARG | B | 332 | 64.279 | 24.347 | 41.983 | 1.00 | 39.76 | C | |
| ATOM | 5827 | CB | ARG | B | 332 | 64.928 | 25.666 | 42.286 | 1.00 | 39.53 | C | |
| ATOM | 5830 | CG | ARG | B | 332 | 64.934 | 26.006 | 43.725 | 1.00 | 39.04 | C | |
| ATOM | 5833 | CD | ARG | B | 332 | 65.345 | 27.407 | 43.966 | 1.00 | 41.30 | C | |
| ATOM | 5836 | NE | ARG | B | 332 | 65.672 | 27.656 | 45.356 | 1.00 | 43.82 | N | |
| ATOM | 5838 | CZ | ARG | B | 332 | 65.843 | 28.878 | 45.889 | 1.00 | 47.86 | C | |
| ATOM | 5839 | NH1 | ARG | B | 332 | 65.742 | 30.022 | 45.172 | 1.00 | 47.83 | N | |
| ATOM | 5842 | NH2 | ARG | B | 332 | 66.159 | 28.966 | 47.172 | 1.00 | 49.85 | N | |
| ATOM | 5845 | C | ARG | B | 332 | 63.972 | 24.290 | 40.483 | 1.00 | 40.24 | C | |
| ATOM | 5846 | O | ARG | B | 332 | 63.041 | 24.939 | 40.018 | 1.00 | 40.19 | O | |
| ATOM | 5848 | N | GLN | B | 333 | 64.733 | 23.503 | 39.724 | 1.00 | 41.33 | N | |
| ATOM | 5849 | CA | GLN | B | 333 | 64.370 | 23.213 | 38.330 | 1.00 | 41.60 | C | |
| ATOM | 5851 | CB | GLN | B | 333 | 65.489 | 22.565 | 37.522 | 1.00 | 40.28 | C | |
| ATOM | 5854 | CG | GLN | B | 333 | 66.789 | 23.316 | 37.497 | 1.00 | 38.01 | C | |
| ATOM | 5857 | CD | GLN | B | 333 | 66.645 | 24.810 | 37.158 | 1.00 | 37.58 | C | |
| ATOM | 5858 | OE1 | GLN | B | 333 | 66.093 | 25.151 | 36.116 | 1.00 | 35.75 | O | |
| ATOM | 5859 | NE2 | GLN | B | 333 | 67.171 | 25.695 | 38.029 | 1.00 | 35.24 | N | |
| ATOM | 5862 | C | GLN | B | 333 | 63.105 | 22.376 | 38.286 | 1.00 | 42.51 | C | |
| ATOM | 5863 | O | GLN | B | 333 | 62.363 | 22.545 | 37.327 | 1.00 | 43.43 | O | |
| ATOM | 5865 | N | GLU | B | 334 | 62.842 | 21.555 | 39.325 | 1.00 | 43.09 | N | |
| ATOM | 5866 | CA | GLU | B | 334 | 61.606 | 20.735 | 39.448 | 1.00 | 44.04 | C | |
| ATOM | 5868 | CB | GLU | B | 334 | 61.828 | 19.470 | 40.279 | 1.00 | 44.33 | C | |
| ATOM | 5871 | CG | GLU | B | 334 | 62.490 | 18.317 | 39.555 | 1.00 | 46.10 | C | |
| ATOM | 5874 | CD | GLU | B | 334 | 62.823 | 17.154 | 40.514 | 1.00 | 48.08 | C | |
| ATOM | 5875 | OE1 | GLU | B | 334 | 62.444 | 17.225 | 41.719 | 1.00 | 55.37 | O | |
| ATOM | 5876 | OE2 | GLU | B | 334 | 63.442 | 16.147 | 40.080 | 1.00 | 50.14 | O | |
| ATOM | 5877 | C | GLU | B | 334 | 60.440 | 21.485 | 40.078 | 1.00 | 43.28 | C | |
| ATOM | 5878 | O | GLU | B | 334 | 59.386 | 21.585 | 39.471 | 1.00 | 44.76 | O | |
| ATOM | 5880 | N | VAL | B | 335 | 60.610 | 21.957 | 41.298 | 1.00 | 42.40 | N | |
| ATOM | 5881 | CA | VAL | B | 335 | 59.660 | 22.865 | 41.981 | 1.00 | 42.46 | C | |
| ATOM | 5883 | CB | VAL | B | 335 | 59.798 | 22.721 | 43.549 | 1.00 | 42.61 | C | |
| ATOM | 5885 | CG1 | VAL | B | 335 | 58.921 | 23.659 | 44.308 | 1.00 | 42.12 | C | |
| ATOM | 5889 | CG2 | VAL | B | 335 | 59.534 | 21.219 | 43.992 | 1.00 | 43.74 | C | |
| ATOM | 5893 | C | VAL | B | 335 | 59.942 | 24.319 | 41.437 | 1.00 | 42.51 | C | |
| ATOM | 5894 | O | VAL | B | 335 | 60.405 | 25.248 | 42.120 | 1.00 | 41.41 | O | |
| ATOM | 5896 | N | ASN | B | 336 | 59.612 | 24.459 | 40.166 | 1.00 | 42.36 | N | |
| ATOM | 5897 | CA | ASN | B | 336 | 59.837 | 25.615 | 39.388 | 1.00 | 42.47 | C | |
| ATOM | 5899 | CB | ASN | B | 336 | 59.970 | 25.155 | 37.927 | 1.00 | 44.16 | C | |
| ATOM | 5902 | CG | ASN | B | 336 | 58.659 | 24.742 | 37.293 | 1.00 | 46.84 | C | |
| ATOM | 5903 | OD1 | ASN | B | 336 | 57.598 | 24.835 | 37.910 | 1.00 | 55.00 | O | |
| ATOM | 5904 | ND2 | ASN | B | 336 | 58.724 | 24.310 | 36.036 | 1.00 | 50.06 | N | |
| ATOM | 5907 | C | ASN | B | 336 | 58.704 | 26.570 | 39.596 | 1.00 | 41.56 | C | |
| ATOM | 5908 | O | ASN | B | 336 | 57.872 | 26.321 | 40.421 | 1.00 | 40.53 | O | |
| ATOM | 5910 | N | ALA | B | 337 | 58.638 | 27.626 | 38.792 | 1.00 | 42.68 | N | |
| ATOM | 5911 | CA | ALA | B | 337 | 57.631 | 28.727 | 38.966 | 1.00 | 42.87 | C | |
| ATOM | 5913 | CB | ALA | B | 337 | 57.964 | 29.860 | 38.099 | 1.00 | 42.22 | C | |
| ATOM | 5917 | C | ALA | B | 337 | 56.167 | 28.344 | 38.756 | 1.00 | 43.30 | C | |
| ATOM | 5918 | O | ALA | B | 337 | 55.346 | 28.765 | 39.533 | 1.00 | 43.77 | O | |
| ATOM | 5920 | N | VAL | B | 338 | 55.848 | 27.533 | 37.738 | 1.00 | 44.16 | N | |
| ATOM | 5921 | CA | VAL | B | 338 | 54.505 | 26.912 | 37.614 | 1.00 | 44.19 | C | |
| ATOM | 5923 | CB | VAL | B | 338 | 54.259 | 26.004 | 36.301 | 1.00 | 44.55 | C | |
| ATOM | 5925 | CG1 | VAL | B | 338 | 54.770 | 26.659 | 35.022 | 1.00 | 44.40 | C | |
| ATOM | 5929 | CG2 | VAL | B | 338 | 54.826 | 24.581 | 36.388 | 1.00 | 44.72 | C | |
| ATOM | 5933 | C | VAL | B | 338 | 54.100 | 26.078 | 38.845 | 1.00 | 44.51 | C | |
| ATOM | 5934 | O | VAL | B | 338 | 52.889 | 26.042 | 39.199 | 1.00 | 45.32 | O | |
| ATOM | 5936 | N | VAL | B | 339 | 55.065 | 25.389 | 39.473 | 1.00 | 43.40 | N | |
| ATOM | 5937 | CA | VAL | B | 339 | 54.722 | 24.623 | 40.691 | 1.00 | 43.21 | C | |
| ATOM | 5939 | CB | VAL | B | 339 | 55.724 | 23.478 | 41.042 | 1.00 | 42.87 | C | |
| ATOM | 5941 | CG1 | VAL | B | 339 | 55.446 | 22.861 | 42.423 | 1.00 | 42.90 | C | |
| ATOM | 5945 | CG2 | VAL | B | 339 | 55.642 | 22.382 | 39.993 | 1.00 | 40.89 | C | |
| ATOM | 5949 | C | VAL | B | 339 | 54.457 | 25.598 | 41.821 | 1.00 | 43.20 | C | |
| ATOM | 5950 | O | VAL | B | 339 | 53.534 | 25.366 | 42.580 | 1.00 | 43.94 | O | |
| ATOM | 5952 | N | LEU | B | 340 | 55.158 | 26.738 | 41.874 | 1.00 | 43.09 | N | |
| ATOM | 5953 | CA | LEU | B | 340 | 54.842 | 27.770 | 42.888 | 1.00 | 42.40 | C | |
| ATOM | 5955 | CB | LEU | B | 340 | 55.845 | 28.926 | 42.934 | 1.00 | 41.12 | C | |
| ATOM | 5958 | CG | LEU | B | 340 | 57.322 | 28.614 | 43.315 | 1.00 | 39.97 | C | |
| ATOM | 5960 | CD1 | LEU | B | 340 | 58.098 | 29.797 | 43.795 | 1.00 | 38.85 | C | |
| ATOM | 5964 | CD2 | LEU | B | 340 | 57.498 | 27.526 | 44.373 | 1.00 | 42.87 | C | |
| ATOM | 5968 | C | LEU | B | 340 | 53.406 | 28.209 | 42.740 | 1.00 | 43.14 | C | |
| ATOM | 5969 | O | LEU | B | 340 | 52.626 | 28.062 | 43.675 | 1.00 | 44.25 | O | |
| ATOM | 5971 | N | LEU | B | 341 | 53.017 | 28.593 | 41.539 | 1.00 | 44.01 | N | |
| ATOM | 5972 | CA | LEU | B | 341 | 51.602 | 28.939 | 41.251 | 1.00 | 44.25 | C | |
| ATOM | 5974 | CB | LEU | B | 341 | 51.427 | 29.244 | 39.772 | 1.00 | 44.45 | C | |
| ATOM | 5977 | CG | LEU | B | 341 | 51.424 | 30.705 | 39.334 | 1.00 | 44.97 | C | |
| ATOM | 5979 | CD1 | LEU | B | 341 | 51.875 | 31.764 | 40.380 | 1.00 | 45.03 | C | |
| ATOM | 5983 | CD2 | LEU | B | 341 | 52.213 | 30.804 | 38.040 | 1.00 | 44.69 | C | |
| ATOM | 5987 | C | LEU | B | 341 | 50.565 | 27.911 | 41.613 | 1.00 | 44.44 | C | |
| ATOM | 5988 | O | LEU | B | 341 | 49.511 | 28.255 | 42.131 | 1.00 | 44.14 | O | |
| ATOM | 5990 | N | TYR | B | 342 | 50.854 | 26.660 | 41.298 | 1.00 | 45.44 | N | |
| ATOM | 5991 | CA | TYR | B | 342 | 49.938 | 25.570 | 41.607 | 1.00 | 46.41 | C | |
| ATOM | 5993 | CB | TYR | B | 342 | 50.537 | 24.274 | 41.136 | 1.00 | 46.60 | C | |
| ATOM | 5996 | CG | TYR | B | 342 | 49.769 | 23.017 | 41.450 | 1.00 | 46.39 | C | |
| ATOM | 5997 | CD1 | TYR | B | 342 | 48.386 | 22.919 | 41.265 | 1.00 | 46.14 | C | |
| ATOM | 5999 | CE1 | TYR | B | 342 | 47.700 | 21.707 | 41.523 | 1.00 | 46.60 | C | |
| ATOM | 6001 | CZ | TYR | B | 342 | 48.425 | 20.600 | 41.977 | 1.00 | 49.02 | C | |
| ATOM | 6002 | OH | TYR | B | 342 | 47.880 | 19.372 | 42.285 | 1.00 | 48.62 | O | |
| ATOM | 6004 | CE2 | TYR | B | 342 | 49.798 | 20.703 | 42.174 | 1.00 | 50.27 | C | |
| ATOM | 6006 | CD2 | TYR | B | 342 | 50.452 | 21.898 | 41.905 | 1.00 | 48.65 | C | |
| ATOM | 6008 | C | TYR | B | 342 | 49.651 | 25.518 | 43.104 | 1.00 | 47.73 | C | |
| ATOM | 6009 | O | TYR | B | 342 | 48.497 | 25.476 | 43.512 | 1.00 | 48.70 | O | |
| ATOM | 6011 | N | MET | B | 343 | 50.711 | 25.573 | 43.904 | 1.00 | 48.51 | N | |
| ATOM | 6012 | CA | MET | B | 343 | 50.605 | 25.521 | 45.352 | 1.00 | 49.05 | C | |
| ATOM | 6014 | CB | MET | B | 343 | 51.991 | 25.524 | 46.003 | 1.00 | 49.15 | C | |
| ATOM | 6017 | CG | MET | B | 343 | 52.855 | 24.302 | 45.668 | 1.00 | 50.17 | C | |
| ATOM | 6020 | SD | MET | B | 343 | 54.574 | 24.212 | 46.372 | 1.00 | 52.32 | S | |
| ATOM | 6021 | CE | MET | B | 343 | 54.747 | 25.849 | 47.112 | 1.00 | 52.41 | C | |
| ATOM | 6025 | C | MET | B | 343 | 49.757 | 26.684 | 45.876 | 1.00 | 48.46 | C | |
| ATOM | 6026 | O | MET | B | 343 | 48.854 | 26.447 | 46.683 | 1.00 | 48.89 | O | |
| ATOM | 6028 | N | ALA | B | 344 | 49.986 | 27.908 | 45.391 | 1.00 | 47.96 | N | |
| ATOM | 6029 | CA | ALA | B | 344 | 49.137 | 29.068 | 45.812 | 1.00 | 48.14 | C | |
| ATOM | 6031 | CB | ALA | B | 344 | 49.692 | 30.374 | 45.307 | 1.00 | 47.60 | C | |
| ATOM | 6035 | C | ALA | B | 344 | 47.647 | 28.936 | 45.416 | 1.00 | 47.93 | C | |
| ATOM | 6036 | O | ALA | B | 344 | 46.763 | 29.323 | 46.171 | 1.00 | 46.98 | O | |
| ATOM | 6038 | N | THR | B | 345 | 47.394 | 28.386 | 44.229 | 1.00 | 48.52 | N | |
| ATOM | 6039 | CA | THR | B | 345 | 46.020 | 28.110 | 43.751 | 1.00 | 48.85 | C | |
| ATOM | 6041 | CB | THR | B | 345 | 46.072 | 27.503 | 42.305 | 1.00 | 49.34 | C | |
| ATOM | 6043 | OG1 | THR | B | 345 | 46.775 | 28.402 | 41.425 | 1.00 | 50.75 | O | |
| ATOM | 6045 | CG2 | THR | B | 345 | 44.662 | 27.353 | 41.681 | 1.00 | 49.27 | C | |
| ATOM | 6049 | C | THR | B | 345 | 45.299 | 27.147 | 44.682 | 1.00 | 48.53 | C | |
| ATOM | 6050 | O | THR | B | 345 | 44.197 | 27.403 | 45.101 | 1.00 | 48.04 | O | |
| ATOM | 6052 | N | GLN | B | 346 | 45.974 | 26.037 | 44.968 | 1.00 | 48.90 | N | |
| ATOM | 6053 | CA | GLN | B | 346 | 45.546 | 25.016 | 45.902 | 1.00 | 48.82 | C | |
| ATOM | 6055 | CB | GLN | B | 346 | 46.674 | 24.013 | 46.063 | 1.00 | 48.55 | C | |
| ATOM | 6058 | CG | GLN | B | 346 | 46.773 | 23.068 | 44.929 | 1.00 | 49.36 | C | |
| ATOM | 6061 | CD | GLN | B | 346 | 47.798 | 22.005 | 45.183 | 1.00 | 50.50 | C | |
| ATOM | 6062 | OE1 | GLN | B | 346 | 47.441 | 20.937 | 45.655 | 1.00 | 49.75 | O | |
| ATOM | 6063 | NE2 | GLN | B | 346 | 49.079 | 22.303 | 44.919 | 1.00 | 50.51 | N | |
| ATOM | 6066 | C | GLN | B | 346 | 45.158 | 25.569 | 47.264 | 1.00 | 48.96 | C | |
| ATOM | 6067 | O | GLN | B | 346 | 44.048 | 25.340 | 47.764 | 1.00 | 48.28 | O | |
| ATOM | 6069 | N | ILE | B | 347 | 46.077 | 26.325 | 47.840 | 1.00 | 48.93 | N | |
| ATOM | 6070 | CA | ILE | B | 347 | 45.830 | 26.931 | 49.139 | 1.00 | 49.62 | C | |
| ATOM | 6072 | CB | ILE | B | 347 | 47.162 | 27.485 | 49.770 | 1.00 | 49.74 | C | |
| ATOM | 6074 | CG | ILE | B | 347 | 48.200 | 26.360 | 49.961 | 1.00 | 50.81 | C | |
| ATOM | 6077 | CD1 | ILE | B | 347 | 49.677 | 26.934 | 50.135 | 1.00 | 51.35 | C | |
| ATOM | 6081 | CG2 | ILE | B | 347 | 46.926 | 28.232 | 51.120 | 1.00 | 48.40 | C | |
| ATOM | 6085 | C | ILE | B | 347 | 44.723 | 28.029 | 49.076 | 1.00 | 49.56 | C | |
| ATOM | 6086 | O | ILE | B | 347 | 43.999 | 28.147 | 50.049 | 1.00 | 50.95 | O | |
| ATOM | 6088 | N | SER | B | 348 | 44.594 | 28.819 | 47.999 | 1.00 | 48.93 | N | |
| ATOM | 6089 | CA | SER | B | 348 | 43.551 | 29.886 | 47.934 | 1.00 | 49.05 | C | |
| ATOM | 6091 | CB | SER | B | 348 | 43.887 | 30.951 | 46.913 | 1.00 | 48.89 | C | |
| ATOM | 6094 | OG | SER | B | 348 | 43.851 | 30.401 | 45.627 | 1.00 | 48.89 | O | |
| ATOM | 6096 | C | SER | B | 348 | 42.145 | 29.347 | 47.632 | 1.00 | 49.20 | C | |
| ATOM | 6097 | O | SER | B | 348 | 41.154 | 29.951 | 48.032 | 1.00 | 49.48 | O | |
| ATOM | 6099 | N | SER | B | 349 | 42.074 | 28.242 | 46.890 | 1.00 | 49.19 | N | |
| ATOM | 6100 | CA | SER | B | 349 | 40.882 | 27.372 | 46.803 | 1.00 | 49.07 | C | |
| ATOM | 6102 | CB | SER | B | 349 | 41.258 | 26.118 | 45.992 | 1.00 | 49.03 | C | |
| ATOM | 6105 | OG | SER | B | 349 | 40.277 | 25.100 | 45.988 | 1.00 | 49.12 | O | |
| ATOM | 6107 | C | SER | B | 349 | 40.283 | 26.988 | 48.196 | 1.00 | 48.88 | C | |
| ATOM | 6108 | O | SER | B | 349 | 39.081 | 27.181 | 48.446 | 1.00 | 48.10 | O | |
| ATOM | 6110 | N | ALA | B | 350 | 41.140 | 26.468 | 49.072 | 1.00 | 48.93 | N | |
| ATOM | 6111 | CA | ALA | B | 350 | 40.793 | 26.139 | 50.472 | 1.00 | 49.59 | C | |
| ATOM | 6113 | CB | ALA | B | 350 | 41.998 | 25.554 | 51.173 | 1.00 | 49.82 | C | |
| ATOM | 6117 | C | ALA | B | 350 | 40.272 | 27.304 | 51.317 | 1.00 | 50.55 | C | |
| ATOM | 6118 | O | ALA | B | 350 | 39.359 | 27.153 | 52.084 | 1.00 | 50.92 | O | |
| ATOM | 6120 | N | MET | B | 351 | 40.875 | 28.465 | 51.196 | 1.00 | 51.81 | N | |
| ATOM | 6121 | CA | MET | B | 351 | 40.407 | 29.631 | 51.948 | 1.00 | 53.08 | C | |
| ATOM | 6123 | CB | MET | B | 351 | 41.488 | 30.702 | 51.953 | 1.00 | 53.27 | C | |
| ATOM | 6126 | CG | MET | B | 351 | 42.854 | 30.281 | 52.488 | 1.00 | 53.87 | C | |
| ATOM | 6129 | SD | MET | B | 351 | 42.848 | 29.434 | 54.059 | 1.00 | 56.31 | S | |
| ATOM | 6130 | CE | MET | B | 351 | 41.865 | 30.640 | 55.056 | 1.00 | 56.23 | C | |
| ATOM | 6134 | C | MET | B | 351 | 39.088 | 30.222 | 51.435 | 1.00 | 53.48 | C | |
| ATOM | 6135 | O | MET | B | 351 | 38.281 | 30.708 | 52.223 | 1.00 | 53.66 | O | |
| ATOM | 6137 | N | GLU | B | 352 | 38.906 | 30.206 | 50.111 | 1.00 | 53.97 | N | |
| ATOM | 6138 | CA | GLU | B | 352 | 37.602 | 30.514 | 49.439 | 1.00 | 53.40 | C | |
| ATOM | 6140 | CB | GLU | B | 352 | 37.771 | 30.494 | 47.890 | 1.00 | 53.34 | C | |
| ATOM | 6143 | CG | GLU | B | 352 | 36.566 | 30.105 | 47.048 | 1.00 | 54.31 | C | |
| ATOM | 6146 | CD | GLU | B | 352 | 36.716 | 30.498 | 45.569 | 1.00 | 56.26 | C | |
| ATOM | 6147 | OE1 | GLU | B | 352 | 37.489 | 29.828 | 44.818 | 1.00 | 59.72 | O | |
| ATOM | 6148 | OE2 | GLU | B | 352 | 36.030 | 31.469 | 45.138 | 1.00 | 59.48 | O | |
| ATOM | 6149 | C | GLU | B | 352 | 36.469 | 29.597 | 49.941 | 1.00 | 52.20 | C | |
| ATOM | 6150 | O | GLU | B | 352 | 35.371 | 30.066 | 50.161 | 1.00 | 51.51 | O | |
| ATOM | 6152 | N | TYR | B | 353 | 36.770 | 28.315 | 50.156 | 1.00 | 51.58 | N | |
| ATOM | 6153 | CA | TYR | B | 353 | 35.809 | 27.337 | 50.729 | 1.00 | 51.45 | C | |
| ATOM | 6155 | CB | TYR | B | 353 | 36.384 | 25.923 | 50.699 | 1.00 | 51.05 | C | |
| ATOM | 6158 | CG | TYR | B | 353 | 35.607 | 24.901 | 51.471 | 1.00 | 51.39 | C | |
| ATOM | 6159 | CD1 | TYR | B | 353 | 34.509 | 24.255 | 50.895 | 1.00 | 51.40 | C | |
| ATOM | 6161 | CE1 | TYR | B | 353 | 33.768 | 23.290 | 51.592 | 1.00 | 50.75 | C | |
| ATOM | 6163 | CZ | TYR | B | 353 | 34.129 | 22.941 | 52.873 | 1.00 | 51.95 | C | |
| ATOM | 6164 | OH | TYR | B | 353 | 33.369 | 21.992 | 53.527 | 1.00 | 52.48 | O | |
| ATOM | 6166 | CE2 | TYR | B | 353 | 35.220 | 23.568 | 53.492 | 1.00 | 52.52 | C | |
| ATOM | 6168 | CD2 | TYR | B | 353 | 35.961 | 24.557 | 52.773 | 1.00 | 52.17 | C | |
| ATOM | 6170 | C | TYR | B | 353 | 35.437 | 27.694 | 52.156 | 1.00 | 51.34 | C | |
| ATOM | 6171 | O | TYR | B | 353 | 34.274 | 27.788 | 52.494 | 1.00 | 51.21 | O | |
| ATOM | 6173 | N | LEU | B | 354 | 36.451 | 27.882 | 52.990 | 1.00 | 51.70 | N | |
| ATOM | 6174 | CA | LEU | B | 354 | 36.257 | 28.360 | 54.360 | 1.00 | 51.19 | C | |
| ATOM | 6176 | CB | LEU | B | 354 | 37.551 | 28.250 | 55.193 | 1.00 | 51.56 | C | |
| ATOM | 6179 | CG | LEU | B | 354 | 38.239 | 26.878 | 55.370 | 1.00 | 51.86 | C | |
| ATOM | 6181 | CD1 | LEU | B | 354 | 39.385 | 27.006 | 56.340 | 1.00 | 51.42 | C | |
| ATOM | 6185 | CD2 | LEU | B | 354 | 37.307 | 25.780 | 55.855 | 1.00 | 53.30 | C | |
| ATOM | 6189 | C | LEU | B | 354 | 35.698 | 29.771 | 54.429 | 1.00 | 50.03 | C | |
| ATOM | 6190 | O | LEU | B | 354 | 35.057 | 30.068 | 55.402 | 1.00 | 49.16 | O | |
| ATOM | 6192 | N | GLU | B | 355 | 35.897 | 30.600 | 53.411 | 1.00 | 49.71 | N | |
| ATOM | 6193 | CA | GLU | B | 355 | 35.271 | 31.952 | 53.373 | 1.00 | 50.98 | C | |
| ATOM | 6195 | CB | GLU | B | 355 | 35.909 | 32.823 | 52.275 | 1.00 | 50.57 | C | |
| ATOM | 6198 | CG | GLU | B | 355 | 35.242 | 34.151 | 51.974 | 1.00 | 50.42 | C | |
| ATOM | 6201 | CD | GLU | B | 355 | 36.038 | 34.989 | 50.948 | 1.00 | 52.55 | C | |
| ATOM | 6202 | OE1 | GLU | B | 355 | 35.944 | 34.744 | 49.697 | 1.00 | 52.19 | O | |
| ATOM | 6203 | OE2 | GLU | B | 355 | 36.759 | 35.920 | 51.400 | 1.00 | 54.21 | O | |
| ATOM | 6204 | C | GLU | B | 355 | 33.743 | 31.823 | 53.191 | 1.00 | 51.27 | C | |
| ATOM | 6205 | O | GLU | B | 355 | 32.951 | 32.272 | 54.019 | 1.00 | 50.91 | O | |
| ATOM | 6207 | N | LYS | B | 356 | 33.367 | 31.153 | 52.114 | 1.00 | 52.39 | N | |
| ATOM | 6208 | CA | LYS | B | 356 | 31.977 | 30.731 | 51.825 | 1.00 | 53.31 | C | |
| ATOM | 6210 | CB | LYS | B | 356 | 31.986 | 29.672 | 50.687 | 1.00 | 52.80 | C | |
| ATOM | 6213 | CG | LYS | B | 356 | 30.627 | 29.364 | 50.137 | 1.00 | 53.68 | C | |
| ATOM | 6216 | CD | LYS | B | 356 | 30.670 | 28.890 | 48.684 | 1.00 | 55.06 | C | |
| ATOM | 6219 | CE | LYS | B | 356 | 29.532 | 29.459 | 47.790 | 1.00 | 56.66 | C | |
| ATOM | 6222 | NZ | LYS | B | 356 | 29.865 | 29.136 | 46.331 | 1.00 | 56.17 | N | |
| ATOM | 6226 | C | LYS | B | 356 | 31.190 | 30.186 | 53.043 | 1.00 | 53.11 | C | |
| ATOM | 6227 | O | LYS | B | 356 | 30.049 | 30.609 | 53.262 | 1.00 | 52.88 | O | |
| ATOM | 6229 | N | LYS | B | 357 | 31.814 | 29.269 | 53.805 | 1.00 | 52.97 | N | |
| ATOM | 6230 | CA | LYS | B | 357 | 31.265 | 28.740 | 55.068 | 1.00 | 53.04 | C | |
| ATOM | 6232 | CB | LYS | B | 357 | 31.754 | 27.318 | 55.300 | 1.00 | 53.30 | C | |
| ATOM | 6235 | CG | LYS | B | 357 | 31.395 | 26.364 | 54.178 | 1.00 | 53.20 | C | |
| ATOM | 6238 | CD | LYS | B | 357 | 31.640 | 24.927 | 54.581 | 1.00 | 53.36 | C | |
| ATOM | 6241 | CE | LYS | B | 357 | 30.365 | 24.066 | 54.751 | 1.00 | 55.93 | C | |
| ATOM | 6244 | NZ | LYS | B | 357 | 30.544 | 22.695 | 54.049 | 1.00 | 56.88 | N | |
| ATOM | 6248 | C | LYS | B | 357 | 31.533 | 29.566 | 56.335 | 1.00 | 53.17 | C | |
| ATOM | 6249 | O | LYS | B | 357 | 31.205 | 29.120 | 57.439 | 1.00 | 52.57 | O | |
| ATOM | 6251 | N | ASN | B | 358 | 32.102 | 30.770 | 56.165 | 1.00 | 53.61 | N | |
| ATOM | 6252 | CA | ASN | B | 358 | 32.346 | 31.762 | 57.223 | 1.00 | 53.33 | C | |
| ATOM | 6254 | CB | ASN | B | 358 | 30.996 | 32.319 | 57.722 | 1.00 | 53.57 | C | |
| ATOM | 6257 | CG | ASN | B | 358 | 31.061 | 33.796 | 58.198 | 1.00 | 54.20 | C | |
| ATOM | 6258 | OD1 | ASN | B | 358 | 31.934 | 34.571 | 57.823 | 1.00 | 55.77 | O | |
| ATOM | 6259 | ND2 | ASN | B | 358 | 30.100 | 34.174 | 59.025 | 1.00 | 55.88 | N | |
| ATOM | 6262 | C | ASN | B | 358 | 33.235 | 31.243 | 58.373 | 1.00 | 53.42 | C | |
| ATOM | 6263 | O | ASN | B | 358 | 33.070 | 31.643 | 59.528 | 1.00 | 54.20 | O | |
| ATOM | 6265 | N | PHE | B | 359 | 34.180 | 30.360 | 58.033 | 1.00 | 53.10 | N | |
| ATOM | 6266 | CA | PHE | B | 359 | 35.257 | 29.919 | 58.931 | 1.00 | 52.82 | C | |
| ATOM | 6268 | CB | PHE | B | 359 | 35.672 | 28.475 | 58.604 | 1.00 | 54.17 | C | |
| ATOM | 6271 | CG | PHE | B | 359 | 34.776 | 27.459 | 59.203 | 1.00 | 55.27 | C | |
| ATOM | 6272 | CD1 | PHE | B | 359 | 35.010 | 26.992 | 60.490 | 1.00 | 58.03 | C | |
| ATOM | 6274 | CE1 | PHE | B | 359 | 34.145 | 26.029 | 61.088 | 1.00 | 58.89 | C | |
| ATOM | 6276 | CZ | PHE | B | 359 | 33.018 | 25.528 | 60.384 | 1.00 | 57.33 | C | |
| ATOM | 6278 | CE2 | PHE | B | 359 | 32.776 | 25.992 | 59.078 | 1.00 | 58.10 | C | |
| ATOM | 6280 | CD2 | PHE | B | 359 | 33.662 | 26.984 | 58.502 | 1.00 | 58.68 | C | |
| ATOM | 6282 | C | PHE | B | 359 | 36.494 | 30.827 | 58.866 | 1.00 | 51.31 | C | |
| ATOM | 6283 | O | PHE | B | 359 | 36.817 | 31.361 | 57.814 | 1.00 | 50.90 | O | |
| ATOM | 6285 | N | ILE | B | 360 | 37.175 | 30.989 | 60.000 | 1.00 | 50.15 | N | |
| ATOM | 6286 | CA | ILE | B | 360 | 38.554 | 31.492 | 60.022 | 1.00 | 49.77 | C | |
| ATOM | 6288 | CB | ILE | B | 360 | 38.667 | 32.846 | 60.724 | 1.00 | 49.77 | C | |
| ATOM | 6290 | CG1 | ILE | B | 360 | 38.110 | 32.814 | 62.147 | 1.00 | 49.19 | C | |
| ATOM | 6293 | CD1 | ILE | B | 360 | 38.574 | 34.039 | 62.973 | 1.00 | 49.80 | C | |
| ATOM | 6297 | CG2 | ILE | B | 360 | 37.968 | 33.933 | 59.880 | 1.00 | 50.05 | C | |
| ATOM | 6301 | C | ILE | B | 360 | 39.536 | 30.427 | 60.580 | 1.00 | 49.72 | C | |
| ATOM | 6302 | O | ILE | B | 360 | 39.151 | 29.571 | 61.365 | 1.00 | 49.63 | O | |
| ATOM | 6304 | N | HIS | B | 361 | 40.780 | 30.469 | 60.097 | 1.00 | 49.57 | N | |
| ATOM | 6305 | CA | HIS | B | 361 | 41.831 | 29.491 | 60.399 | 1.00 | 49.53 | C | |
| ATOM | 6307 | CB | HIS | B | 361 | 42.713 | 29.247 | 59.156 | 1.00 | 49.65 | C | |
| ATOM | 6310 | CG | HIS | B | 361 | 43.741 | 28.165 | 59.336 | 1.00 | 49.95 | C | |
| ATOM | 6311 | ND1 | HIS | B | 361 | 45.051 | 28.417 | 59.704 | 1.00 | 48.80 | N | |
| ATOM | 6313 | CE1 | HIS | B | 361 | 45.706 | 27.271 | 59.800 | 1.00 | 50.27 | C | |
| ATOM | 6315 | NE2 | HIS | B | 361 | 44.879 | 26.292 | 59.479 | 1.00 | 51.03 | N | |
| ATOM | 6317 | CD2 | HIS | B | 361 | 43.640 | 26.823 | 59.201 | 1.00 | 49.19 | C | |
| ATOM | 6319 | C | HIS | B | 361 | 42.712 | 29.993 | 61.516 | 1.00 | 49.42 | C | |
| ATOM | 6320 | O | HIS | B | 361 | 43.087 | 29.231 | 62.399 | 1.00 | 49.10 | O | |
| ATOM | 6322 | N | ARG | B | 362 | 43.124 | 31.250 | 61.392 | 1.00 | 49.61 | N | |
| ATOM | 6323 | CA | ARG | B | 362 | 43.966 | 31.971 | 62.352 | 1.00 | 50.28 | C | |
| ATOM | 6325 | CB | ARG | B | 362 | 43.237 | 32.151 | 63.676 | 1.00 | 49.95 | C | |
| ATOM | 6328 | CG | ARG | B | 362 | 42.136 | 33.185 | 63.627 | 1.00 | 50.78 | C | |
| ATOM | 6331 | CD | ARG | B | 362 | 41.712 | 33.622 | 64.985 | 1.00 | 49.91 | C | |
| ATOM | 6334 | NE | ARG | B | 362 | 41.403 | 32.439 | 65.765 | 1.00 | 48.95 | N | |
| ATOM | 6336 | CZ | ARG | B | 362 | 41.544 | 32.320 | 67.073 | 1.00 | 51.46 | C | |
| ATOM | 6337 | NH1 | ARG | B | 362 41.959 | 33.343 | 67.845 | 1.00 | 51.57 | N | | |
| ATOM | 6340 | NH2 | ARG | B | 362 41.204 | 31.155 | 67.652 | 1.00 | 53.04 | N | | |
| ATOM | 6343 | C | ARG | B | 362 | 45.423 | 31.551 | 62.600 | 1.00 | 50.31 | C | |
| ATOM | 6344 | O | ARG | B | 362 | 46.092 | 32.245 | 63.322 | 1.00 | 50.08 | O | |
| ATOM | 6346 | N | ASP | B | 363 | 45.922 | 30.460 | 62.027 | 1.00 | 51.58 | N | |
| ATOM | 6347 | CA | ASP | B | 363 | 47.366 | 30.115 | 62.113 | 1.00 | 51.84 | C | |
| ATOM | 6349 | CB | ASP | B | 363 | 47.617 | 29.114 | 63.244 | 1.00 | 52.50 | C | |
| ATOM | 6352 | CG | ASP | B | 363 | 49.102 | 28.894 | 63.547 | 1.00 | 53.49 | C | |
| ATOM | 6353 | OD1 | ASP | B | 363 49.938 | 29.812 | 63.306 | 1.00 | 57.02 | O | | |
| ATOM | 6354 | OD2 | ASP | B | 363 | 49.504 | 27.792 | 64.015 | 1.00 | 56.95 | O | |
| ATOM | 6355 | C | ASP | B | 363 | 47.897 | 29.633 | 60.761 | 1.00 | 52.05 | C | |
| ATOM | 6356 | O | ASP | B | 363 | 48.505 | 28.566 | 60.627 | 1.00 | 52.49 | O | |
| ATOM | 6358 | N | LEU | B | 364 | 47.670 | 30.481 | 59.760 | 1.00 | 52.12 | N | |
| ATOM | 6359 | CA | LEU | B | 364 | 48.019 | 30.201 | 58.365 | 1.00 | 51.31 | C | |
| ATOM | 6361 | CB | LEU | B | 364 | 47.076 | 30.978 | 57.462 | 1.00 | 51.80 | C | |
| ATOM | 6364 | CG | LEU | B | 364 | 47.074 | 30.765 | 55.958 | 1.00 | 52.67 | C | |
| ATOM | 6366 | CD1 | LEU | B | 364 | 46.666 | 29.353 | 55.618 | 1.00 | 53.11 | C | |
| ATOM | 6370 | CD2 | LEU | B | 364 | 46.135 | 31.827 | 55.295 | 1.00 | 52.29 | C | |
| ATOM | 6374 | C | LEU | B | 364 | 49.444 | 30.664 | 58.179 | 1.00 | 49.67 | C | |
| ATOM | 6375 | O | LEU | B | 364 | 49.782 | 31.761 | 58.531 | 1.00 | 51.46 | O | |
| ATOM | 6377 | N | ALA | B | 365 | 50.256 | 29.818 | 57.607 | 1.00 | 47.82 | N | |
| ATOM | 6378 | CA | ALA | B | 365 | 51.678 | 29.910 | 57.600 | 1.00 | 46.42 | C | |
| ATOM | 6380 | CB | ALA | B | 365 | 52.218 | 29.956 | 59.018 | 1.00 | 46.55 | C | |
| ATOM | 6384 | C | ALA | B | 365 | 52.179 | 28.635 | 56.894 | 1.00 | 46.17 | C | |
| ATOM | 6385 | O | ALA | B | 365 | 51.540 | 27.601 | 56.905 | 1.00 | 45.40 | O | |
| ATOM | 6387 | N | ALA | B | 366 | 53.338 | 28.741 | 56.295 | 1.00 | 46.80 | N | |
| ATOM | 6388 | CA | ALA | B | 366 | 53.968 | 27.668 | 55.553 | 1.00 | 47.57 | C | |
| ATOM | 6390 | CB | ALA | B | 366 | 55.289 | 28.178 | 54.995 | 1.00 | 48.13 | C | |
| ATOM | 6394 | C | ALA | B | 366 | 54.210 | 26.412 | 56.390 | 1.00 | 47.64 | C | |
| ATOM | 6395 | O | ALA | B | 366 | 53.971 | 25.321 | 55.923 | 1.00 | 46.90 | O | |
| ATOM | 6397 | N | ARG | B | 367 | 54.630 | 26.594 | 57.644 | 1.00 | 48.44 | N | |
| ATOM | 6398 | CA | ARG | B | 367 | 54.877 | 25.484 | 58.591 | 1.00 | 48.55 | C | |
| ATOM | 6400 | CB | ARG | B | 367 | 55.465 | 25.985 | 59.918 | 1.00 | 47.93 | C | |
| ATOM | 6403 | CG | ARG | B | 367 | 54.529 | 26.841 | 60.743 | 1.00 | 48.51 | C | |
| ATOM | 6406 | CD | ARG | B | 367 | 55.139 | 27.555 | 61.948 | 1.00 | 49.87 | C | |
| ATOM | 6409 | NE | ARG | B | 367 | 54.230 | 28.591 | 62.482 | 1.00 | 53.28 | N | |
| ATOM | 6411 | CZ | ARG | B | 367 | 54.195 | 29.908 | 62.124 | 1.00 | 56.39 | C | |
| ATOM | 6412 | NH1 | ARG | B | 367 | 55.028 | 30.401 | 61.219 | 1.00 | 60.35 | N | |
| ATOM | 6415 | NH2 | ARG | B | 367 | 53.327 | 30.753 | 62.691 | 1.00 | 54.63 | N | |
| ATOM | 6418 | C | ARG | B | 367 | 53.637 | 24.628 | 58.867 | 1.00 | 49.35 | C | |
| ATOM | 6419 | O | ARG | B | 367 | 53.770 | 23.501 | 59.339 | 1.00 | 50.07 | O | |
| ATOM | 6421 | N | ASN | B | 368 | 52.456 | 25.170 | 58.577 | 1.00 | 50.19 | N | |
| ATOM | 6422 | CA | ASN | B | 368 | 51.185 | 24.477 | 58.690 | 1.00 | 50.81 | C | |
| ATOM | 6424 | CB | ASN | B | 368 | 50.199 | 25.378 | 59.430 | 1.00 | 51.14 | C | |
| ATOM | 6427 | CG | ASN | B | 368 | 50.632 | 25.657 | 60.800 | 1.00 | 50.73 | C | |
| ATOM | 6428 | OD1 | ASN | B | 368 | 51.332 | 24.859 | 61.397 | 1.00 | 51.15 | O | |
| ATOM | 6429 | ND2 | ASN | B | 368 | 50.247 | 26.803 | 61.320 | 1.00 | 52.40 | N | |
| ATOM | 6432 | C | ASN | B | 368 | 50.574 | 24.057 | 57.376 | 1.00 | 51.32 | C | |
| ATOM | 6433 | O | ASN | B | 368 | 49.427 | 23.572 | 57.371 | 1.00 | 51.72 | O | |
| ATOM | 6435 | N | CYS | B | 369 | 51.291 | 24.253 | 56.271 | 1.00 | 51.29 | N | |
| ATOM | 6436 | CA | CYS | B | 369 | 50.941 | 23.578 | 55.029 | 1.00 | 51.65 | C | |
| ATOM | 6438 | CB | CYS | B | 369 | 51.193 | 24.467 | 53.868 | 1.00 | 51.65 | C | |
| ATOM | 6441 | SG | CYS | B | 369 | 50.343 | 26.038 | 54.159 | 1.00 | 55.03 | S | |
| ATOM | 6443 | C | CYS | B | 369 | 51.767 | 22.321 | 54.967 | 1.00 | 51.55 | C | |
| ATOM | 6444 | O | CYS | B | 369 | 52.938 | 22.302 | 55.365 | 1.00 | 52.33 | O | |
| ATOM | 6446 | N | LEU | B | 370 | 51.129 | 21.250 | 54.536 | 1.00 | 51.32 | N | |
| ATOM | 6447 | CA | LEU | B | 370 | 51.765 | 19.950 | 54.394 | 1.00 | 51.06 | C | |
| ATOM | 6449 | CB | LEU | B | 370 | 51.035 | 18.921 | 55.264 | 1.00 | 50.98 | C | |
| ATOM | 6452 | CG | LEU | B | 370 | 51.435 | 18.640 | 56.729 | 1.00 | 49.93 | C | |
| ATOM | 6454 | CD1 | LEU | B | 370 | 52.580 | 19.493 | 57.203 | 1.00 | 50.05 | C | |
| ATOM | 6458 | CD2 | LEU | B | 370 | 50.261 | 18.733 | 57.695 | 1.00 | 48.45 | C | |
| ATOM | 6462 | C | LEU | B | 370 | 51.717 | 19.555 | 52.947 | 1.00 | 50.86 | C | |
| ATOM | 6463 | O | LEU | B | 370 | 50.873 | 20.029 | 52.230 | 1.00 | 50.99 | O | |
| ATOM | 6465 | N | VAL | B | 371 | 52.619 | 18.680 | 52.530 | 1.00 | 51.51 | N | |
| ATOM | 6466 | CA | VAL | B | 371 | 52.728 | 18.301 | 51.124 | 1.00 | 52.17 | C | |
| ATOM | 6468 | CB | VAL | B | 371 | 54.115 | 18.636 | 50.523 | 1.00 | 52.20 | C | |
| ATOM | 6470 | CG1 | VAL | B | 371 | 54.047 | 18.603 | 48.967 | 1.00 | 50.93 | C | |
| ATOM | 6474 | CG2 | VAL | B | 371 | 54.611 | 20.017 | 51.023 | 1.00 | 51.52 | C | |
| ATOM | 6478 | C | VAL | B | 371 | 52.414 | 16.831 | 50.916 | 1.00 | 52.80 | C | |
| ATOM | 6479 | O | VAL | B | 371 | 52.650 | 15.996 | 51.789 | 1.00 | 53.35 | O | |
| ATOM | 6481 | N | GLY | B | 372 | 51.847 | 16.543 | 49.751 | 1.00 | 53.47 | N | |
| ATOM | 6482 | CA | GLY | B | 372 | 51.628 | 15.198 | 49.269 | 1.00 | 53.68 | C | |
| ATOM | 6485 | C | GLY | B | 372 | 52.151 | 14.945 | 47.857 | 1.00 | 54.31 | C | |
| ATOM | 6486 | O | GLY | B | 372 | 53.022 | 15.660 | 47.308 | 1.00 | 54.45 | O | |
| ATOM | 6488 | N | GLU | B | 373 | 51.587 | 13.887 | 47.282 | 1.00 | 55.20 | N | |
| ATOM | 6489 | CA | GLU | B | 373 | 52.038 | 13.267 | 46.036 | 1.00 | 54.98 | C | |
| ATOM | 6491 | CB | GLU | B | 373 | 51.226 | 11.984 | 45.831 | 1.00 | 55.64 | C | |
| ATOM | 6494 | CG | GLU | B | 373 | 51.939 | 10.896 | 45.093 | 1.00 | 60.09 | C | |
| ATOM | 6497 | CD | GLU | B | 373 | 52.608 | 9.877 | 46.012 | 1.00 | 65.91 | C | |
| ATOM | 6498 | OE1 | GLU | B | 373 | 51.839 | 9.118 | 46.668 | 1.00 | 69.45 | O | |
| ATOM | 6499 | OE2 | GLU | B | 373 | 53.887 | 9.796 | 46.022 | 1.00 | 67.34 | O | |
| ATOM | 6500 | C | GLU | B | 373 | 51.769 | 14.295 | 44.924 | 1.00 | 54.34 | C | |
| ATOM | 6501 | O | GLU | B | 373 | 50.716 | 14.940 | 44.963 | 1.00 | 53.24 | O | |
| ATOM | 6503 | N | ASN | B | 374 | 52.740 | 14.500 | 44.018 | 1.00 | 54.00 | N | |
| ATOM | 6504 | CA | ASN | B | 374 | 52.634 | 15.459 | 42.861 | 1.00 | 53.84 | C | |
| ATOM | 6506 | CB | ASN | B | 374 | 51.625 | 14.915 | 41.841 | 1.00 | 54.18 | C | |
| ATOM | 6509 | CG | ASN | B | 374 | 51.793 | 15.505 | 40.413 | 1.00 | 54.64 | C | |
| ATOM | 6510 | OD1 | ASN | B | 374 | 52.830 | 16.110 | 40.050 | 1.00 | 52.89 | O | |
| ATOM | 6511 | ND2 | ASN | B | 374 | 50.752 | 15.282 | 39.583 | 1.00 | 54.67 | N | |
| ATOM | 6514 | C | ASN | B | 374 | 52.285 | 16.935 | 43.213 | 1.00 | 53.43 | C | |
| ATOM | 6515 | O | ASN | B | 374 | 51.356 | 17.542 | 42.657 | 1.00 | 53.15 | O | |
| ATOM | 6517 | N | HIS | B | 375 | 53.026 | 17.471 | 44.178 | 1.00 | 53.02 | N | |
| ATOM | 6518 | CA | HIS | B | 375 | 52.927 | 18.885 | 44.630 | 1.00 | 52.54 | C | |
| ATOM | 6520 | CB | HIS | B | 375 | 53.354 | 19.854 | 43.499 | 1.00 | 52.15 | C | |
| ATOM | 6523 | CG | HIS | B | 375 | 54.723 | 19.572 | 42.966 | 1.00 | 50.07 | C | |
| ATOM | 6524 | ND1 | HIS | B | 375 | 55.052 | 19.591 | 41.622 | 1.00 | 49.30 | N | |
| ATOM | 6526 | CE1 | HIS | B | 375 | 56.336 | 19.304 | 41.487 | 1.00 | 46.42 | C | |
| ATOM | 6528 | NE2 | HIS | B | 375 | 56.844 | 19.111 | 42.691 | 1.00 | 45.04 | N | |
| ATOM | 6530 | CD2 | HIS | B | 375 | 55.850 | 19.260 | 43.623 | 1.00 | 45.68 | C | |
| ATOM | 6532 | C | HIS | B | 375 | 51.590 | 19.295 | 45.260 | 1.00 | 51.78 | C | |
| ATOM | 6533 | O | HIS | B | 375 | 51.246 | 20.495 | 45.279 | 1.00 | 52.18 | O | |
| ATOM | 6535 | N | LEU | B | 376 | 50.878 | 18.308 | 45.810 | 1.00 | 50.84 | N | |
| ATOM | 6536 | CA | LEU | B | 376 | 49.586 | 18.537 | 46.471 | 1.00 | 50.31 | C | |
| ATOM | 6538 | CB | LEU | B | 376 | 48.863 | 17.225 | 46.712 | 1.00 | 50.27 | C | |
| ATOM | 6541 | CG | LEU | B | 376 | 47.485 | 17.292 | 47.362 | 1.00 | 50.31 | C | |
| ATOM | 6543 | CD1 | LEU | B | 376 | 46.401 | 17.526 | 46.278 | 1.00 | 52.32 | C | |
| ATOM | 6547 | CD2 | LEU | B | 376 | 47.229 | 16.042 | 48.150 | 1.00 | 47.19 | C | |
| ATOM | 6551 | C | LEU | B | 376 | 49.865 | 19.188 | 47.811 | 1.00 | 49.98 | C | |
| ATOM | 6552 | O | LEU | B | 376 | 50.608 | 18.609 | 48.605 | 1.00 | 49.49 | O | |
| ATOM | 6554 | N | VAL | B | 377 | 49.297 | 20.382 | 48.048 | 1.00 | 49.71 | N | |
| ATOM | 6555 | CA | VAL | B | 377 | 49.464 | 21.111 | 49.313 | 1.00 | 48.98 | C | |
| ATOM | 6557 | CB | VAL | B | 377 | 49.951 | 22.505 | 49.065 | 1.00 | 48.72 | C | |
| ATOM | 6559 | CG1 | VAL | B | 377 | 50.284 | 23.171 | 50.389 | 1.00 | 49.65 | C | |
| ATOM | 6563 | CG2 | VAL | B | 377 | 51.186 | 22.473 | 48.230 | 1.00 | 49.10 | C | |
| ATOM | 6567 | C | VAL | B | 377 | 48.121 | 21.139 | 50.057 | 1.00 | 49.03 | C | |
| ATOM | 6568 | O | VAL | B | 377 | 47.067 | 21.367 | 49.430 | 1.00 | 49.97 | O | |
| ATOM | 6570 | N | LYS | B | 378 | 48.139 | 20.909 | 51.368 | 1.00 | 48.50 | N | |
| ATOM | 6571 | CA | LYS | B | 378 | 46.901 | 20.909 | 52.182 | 1.00 | 48.97 | C | |
| ATOM | 6573 | CB | LYS | B | 378 | 46.442 | 19.477 | 52.544 | 1.00 | 49.43 | C | |
| ATOM | 6576 | CG | LYS | B | 378 | 45.891 | 18.589 | 51.382 | 1.00 | 48.53 | C | |
| ATOM | 6579 | CD | LYS | B | 378 | 45.476 | 17.235 | 51.865 | 1.00 | 48.74 | C | |
| ATOM | 6582 | CE | LYS | B | 378 | 44.525 | 16.545 | 50.870 | 1.00 | 50.27 | C | |
| ATOM | 6585 | NZ | LYS | B | 378 | 44.505 | 15.040 | 50.986 | 1.00 | 48.11 | N | |
| ATOM | 6589 | C | LYS | B | 378 | 47.127 | 21.744 | 53.456 | 1.00 | 49.71 | C | |
| ATOM | 6590 | O | LYS | B | 378 | 48.116 | 21.551 | 54.175 | 1.00 | 49.45 | O | |
| ATOM | 6592 | N | VAL | B | 379 | 46.219 | 22.687 | 53.728 | 1.00 | 50.43 | N | |
| ATOM | 6593 | CA | VAL | B | 379 | 46.322 | 23.521 | 54.918 | 1.00 | 50.48 | C | |
| ATOM | 6595 | CB | VAL | B | 379 | 45.451 | 24.765 | 54.819 | 1.00 | 50.54 | C | |
| ATOM | 6597 | CG1 | VAL | B | 379 | 45.571 | 25.628 | 56.115 | 1.00 | 50.67 | C | |
| ATOM | 6601 | CG2 | VAL | B | 379 | 45.875 | 25.595 | 53.617 | 1.00 | 49.61 | C | |
| ATOM | 6605 | C | VAL | B | 379 | 45.927 | 22.643 | 56.096 | 1.00 | 50.97 | C | |
| ATOM | 6606 | O | VAL | B | 379 | 45.002 | 21.844 | 55.964 | 1.00 | 52.24 | O | |
| ATOM | 6608 | N | ALA | B | 380 | 46.649 | 22.776 | 57.213 | 1.00 | 51.10 | N | |
| ATOM | 6609 | CA | ALA | B | 380 | 46.445 | 21.979 | 58.428 | 1.00 | 51.04 | C | |
| ATOM | 6611 | CB | ALA | B | 380 | 47.484 | 20.875 | 58.463 | 1.00 | 51.41 | C | |
| ATOM | 6615 | C | ALA | B | 380 | 46.489 | 22.804 | 59.723 | 1.00 | 51.52 | C | |
| ATOM | 6616 | O | ALA | B | 380 | 46.702 | 24.016 | 59.683 | 1.00 | 52.60 | O | |
| ATOM | 6618 | N | ASP | B | 381 | 46.267 | 22.126 | 60.856 | 1.00 | 51.45 | N | |
| ATOM | 6619 | CA | ASP | B | 381 | 46.149 | 22.705 | 62.208 | 1.00 | 50.86 | C | |
| ATOM | 6621 | CB | ASP | B | 381 | 47.409 | 23.351 | 62.729 | 1.00 | 50.30 | C | |
| ATOM | 6624 | CG | ASP | B | 381 | 47.380 | 23.487 | 64.223 | 1.00 | 51.47 | C | |
| ATOM | 6625 | OD1 | ASP | B | 381 | 46.285 | 23.684 | 64.799 | 1.00 | 54.38 | O | |
| ATOM | 6626 | OD2 | ASP | B | 381 | 48.382 | 23.345 | 64.942 | 1.00 | 53.95 | O | |
| ATOM | 6627 | C | ASP | B | 381 | 44.995 | 23.652 | 62.386 | 1.00 | 51.12 | C | |
| ATOM | 6628 | O | ASP | B | 381 | 45.120 | 24.881 | 62.225 | 1.00 | 51.58 | O | |
| ATOM | 6630 | N | PHE | B | 382 | 43.892 | 23.077 | 62.828 | 1.00 | 51.55 | N | |
| ATOM | 6631 | CA | PHE | B | 382 | 42.697 | 23.851 | 63.157 | 1.00 | 52.00 | C | |
| ATOM | 6633 | CB | PHE | B | 382 | 41.475 | 23.145 | 62.538 | 1.00 | 52.71 | C | |
| ATOM | 6636 | CG | PHE | B | 382 | 41.644 | 22.888 | 61.065 | 1.00 | 53.02 | C | |
| ATOM | 6637 | CD1 | PHE | B | 382 | 41.472 | 23.916 | 60.136 | 1.00 | 53.82 | C | |
| ATOM | 6639 | CE1 | PHE | B | 382 | 41.659 | 23.692 | 58.770 | 1.00 | 54.21 | C | |
| ATOM | 6641 | CZ | PHE | B | 382 | 42.037 | 22.422 | 58.319 | 1.00 | 55.25 | C | |
| ATOM | 6643 | CE2 | PHE | B | 382 | 42.215 | 21.371 | 59.246 | 1.00 | 55.72 | C | |
| ATOM | 6645 | CD2 | PHE | B | 382 | 42.016 | 21.636 | 60.615 | 1.00 | 54.93 | C | |
| ATOM | 6647 | C | PHE | B | 382 | 42.546 | 24.126 | 64.671 | 1.00 | 51.17 | C | |
| ATOM | 6648 | O | PHE | B | 382 | 41.470 | 24.487 | 65.113 | 1.00 | 50.81 | O | |
| ATOM | 6650 | N | GLY | B | 383 | 43.616 | 23.969 | 65.452 | 1.00 | 50.11 | N | |
| ATOM | 6651 | CA | GLY | B | 383 | 43.568 | 24.290 | 66.852 | 1.00 | 50.07 | C | |
| ATOM | 6654 | C | GLY | B | 383 | 42.989 | 25.674 | 67.182 | 1.00 | 50.37 | C | |
| ATOM | 6655 | O | GLY | B | 383 | 42.260 | 25.794 | 68.162 | 1.00 | 50.65 | O | |
| ATOM | 6657 | N | LEU | B | 384 | 43.308 | 26.689 | 66.365 | 1.00 | 50.10 | N | |
| ATOM | 6658 | CA | LEU | B | 384 | 42.789 | 28.045 | 66.481 | 1.00 | 49.79 | C | |
| ATOM | 6660 | CB | LEU | B | 384 | 43.962 | 29.027 | 66.313 | 1.00 | 49.37 | C | |
| ATOM | 6663 | CG | LEU | B | 384 | 45.110 | 28.956 | 67.327 | 1.00 | 48.94 | C | |
| ATOM | 6665 | CD1 | LEU | B | 384 | 46.200 | 29.933 | 66.909 | 1.00 | 46.27 | C | |
| ATOM | 6669 | CD2 | LEU | B | 384 | 44.683 | 29.258 | 68.792 | 1.00 | 47.51 | C | |
| ATOM | 6673 | C | LEU | B | 384 | 41.684 | 28.407 | 65.460 | 1.00 | 50.29 | C | |
| ATOM | 6674 | O | LEU | B | 384 | 41.532 | 29.575 | 65.152 | 1.00 | 51.84 | O | |
| ATOM | 6676 | N | SER | B | 385 | 40.914 | 27.459 | 64.931 | 1.00 | 50.11 | N | |
| ATOM | 6677 | CA | SER | B | 385 | 39.796 | 27.771 | 64.000 | 1.00 | 50.06 | C | |
| ATOM | 6679 | CB | SER | B | 385 | 39.511 | 26.576 | 63.121 | 1.00 | 50.19 | C | |
| ATOM | 6682 | OG | SER | B | 385 | 40.484 | 26.549 | 62.112 | 1.00 | 52.84 | O | |
| ATOM | 6684 | C | SER | B | 385 | 38.492 | 28.155 | 64.674 | 1.00 | 50.19 | C | |
| ATOM | 6685 | O | SER | B | 385 | 38.322 | 27.893 | 65.823 | 1.00 | 50.01 | O | |
| ATOM | 6687 | N | ARG | B | 386 | 37.565 | 28.751 | 63.935 | 1.00 | 50.70 | N | |
| ATOM | 6688 | CA | ARG | B | 386 | 36.320 | 29.245 | 64.495 | 1.00 | 51.56 | C | |
| ATOM | 6690 | CB | ARG | B | 386 | 36.490 | 30.653 | 65.102 | 1.00 | 52.33 | C | |
| ATOM | 6693 | CG | ARG | B | 386 | 37.356 | 30.849 | 66.347 | 1.00 | 53.70 | C | |
| ATOM | 6696 | CD | ARG | B | 386 | 36.924 | 30.111 | 67.622 | 1.00 | 56.86 | C | |
| ATOM | 6699 | NE | ARG | B | 386 | 38.031 | 30.117 | 68.611 | 1.00 | 58.12 | N | |
| ATOM | 6701 | CZ | ARG | B | 386 | 38.727 | 29.053 | 69.062 | 1.00 | 58.91 | C | |
| ATOM | 6702 | NH1 | ARG | B | 386 | 38.488 | 27.799 | 68.667 | 1.00 | 60.42 | N | |
| ATOM | 6705 | NH2 | ARG | B | 386 | 39.681 | 29.254 | 69.960 | 1.00 | 58.46 | N | |
| ATOM | 6708 | C | ARG | B | 386 | 35.250 | 29.385 | 63.430 | 1.00 | 51.77 | C | |
| ATOM | 6709 | O | ARG | B | 386 | 35.504 | 30.031 | 62.435 | 1.00 | 50.96 | O | |
| ATOM | 6711 | N | LEU | B | 387 | 34.049 | 28.840 | 63.685 | 1.00 | 52.61 | N | |
| ATOM | 6712 | CA | LEU | B | 387 | 32.814 | 29.222 | 62.940 | 1.00 | 52.85 | C | |
| ATOM | 6714 | CB | LEU | B | 387 | 31.737 | 28.117 | 63.004 | 1.00 | 53.13 | C | |
| ATOM | 6717 | CG | LEU | B | 387 | 30.313 | 28.476 | 62.473 | 1.00 | 53.38 | C | |
| ATOM | 6719 | CD1 | LEU | B | 387 | 29.917 | 27.755 | 61.206 | 1.00 | 53.46 | C | |
| ATOM | 6723 | CD2 | LEU | B | 387 | 29.265 | 28.208 | 63.541 | 1.00 | 55.37 | C | |
| ATOM | 6727 | C | LEU | B | 387 | 32.215 | 30.545 | 63.461 | 1.00 | 52.49 | C | |
| ATOM | 6728 | O | LEU | B | 387 | 31.588 | 30.577 | 64.502 | 1.00 | 52.24 | O | |
| ATOM | 6730 | N | MET | B | 388 | 32.328 | 31.600 | 62.675 | 1.00 | 52.75 | N | |
| ATOM | 6731 | CA | MET | B | 388 | 31.924 | 32.932 | 63.103 | 1.00 | 52.92 | C | |
| ATOM | 6733 | CB | MET | B | 388 | 32.722 | 33.953 | 62.342 | 1.00 | 52.61 | C | |
| ATOM | 6736 | CG | MET | B | 388 | 34.166 | 33.938 | 62.605 | 1.00 | 51.08 | C | |
| ATOM | 6739 | SD | MET | B | 388 | 34.704 | 35.491 | 61.919 | 1.00 | 53.35 | S | |
| ATOM | 6740 | CE | MET | B | 388 | 34.695 | 35.103 | 60.168 | 1.00 | 54.32 | C | |
| ATOM | 6744 | C | MET | B | 388 | 30.462 | 33.298 | 62.858 | 1.00 | 53.68 | C | |
| ATOM | 6745 | O | MET | B | 388 | 29.956 | 33.085 | 61.771 | 1.00 | 53.92 | O | |
| ATOM | 6747 | N | THR | B | 389 | 29.825 | 33.873 | 63.882 | 1.00 | 54.77 | N | |
| ATOM | 6748 | CA | THR | B | 389 | 28.690 | 34.787 | 63.746 | 1.00 | 55.77 | C | |
| ATOM | 6750 | CB | THR | B | 389 | 27.921 | 34.924 | 65.088 | 1.00 | 55.87 | C | |
| ATOM | 6752 | OG1 | THR | B | 389 | 27.549 | 33.639 | 65.598 | 1.00 | 56.78 | O | |
| ATOM | 6754 | CG2 | THR | B | 389 | 26.577 | 35.630 | 64.905 | 1.00 | 56.50 | C | |
| ATOM | 6758 | C | THR | B | 389 | 29.277 | 36.147 | 63.397 | 1.00 | 56.91 | C | |
| ATOM | 6759 | O | THR | B | 389 | 30.169 | 36.636 | 64.104 | 1.00 | 57.57 | O | |
| ATOM | 6761 | N | GLY | B | 390 | 28.798 | 36.769 | 62.321 | 1.00 | 58.00 | N | |
| ATOM | 6762 | CA | GLY | B | 390 | 29.391 | 38.023 | 61.812 | 1.00 | 58.88 | C | |
| ATOM | 6765 | C | GLY | B | 390 | 30.722 | 37.727 | 61.148 | 1.00 | 59.36 | C | |
| ATOM | 6766 | O | GLY | B | 390 | 31.070 | 36.552 | 61.018 | 1.00 | 59.78 | O | |
| ATOM | 6768 | N | ASP | B | 391 | 31.464 | 38.759 | 60.724 | 1.00 | 60.07 | N | |
| ATOM | 6769 | CA | ASP | B | 391 | 32.888 | 38.567 | 60.320 | 1.00 | 60.19 | C | |
| ATOM | 6771 | CB | ASP | B | 391 | 33.184 | 39.124 | 58.896 | 1.00 | 61.19 | C | |
| ATOM | 6774 | CG | ASP | B | 391 | 34.574 | 38.627 | 58.296 | 1.00 | 61.68 | C | |
| ATOM | 6775 | OD1 | ASP | B | 391 | 34.740 | 37.375 | 58.117 | 1.00 | 62.75 | O | |
| ATOM | 6776 | OD2 | ASP | B | 391 | 35.521 | 39.433 | 57.957 | 1.00 | 61.66 | O | |
| ATOM | 6777 | C | ASP | B | 391 | 33.831 | 39.157 | 61.353 | 1.00 | 59.13 | C | |
| ATOM | 6778 | O | ASP | B | 391 | 34.836 | 39.765 | 61.005 | 1.00 | 60.25 | O | |
| ATOM | 6780 | N | THR | B | 392 | 33.504 | 39.004 | 62.630 | 1.00 | 57.77 | N | |
| ATOM | 6781 | CA | THR | B | 392 | 34.487 | 39.166 | 63.691 | 1.00 | 56.07 | C | |
| ATOM | 6783 | CB | THR | B | 392 | 34.283 | 40.466 | 64.415 | 1.00 | 55.55 | C | |
| ATOM | 6785 | OG1 | THR | B | 392 | 34.437 | 41.524 | 63.475 | 1.00 | 54.07 | O | |
| ATOM | 6787 | CG2 | THR | B | 392 | 35.391 | 40.737 | 65.435 | 1.00 | 54.91 | C | |
| ATOM | 6791 | C | THR | B | 392 | 34.437 | 37.958 | 64.623 | 1.00 | 55.94 | C | |
| ATOM | 6792 | O | THR | B | 392 | 33.358 | 37.354 | 64.878 | 1.00 | 55.72 | O | |
| ATOM | 6794 | N | TYR | B | 393 | 35.643 | 37.548 | 65.011 | 1.00 | 55.20 | N | |
| ATOM | 6795 | CA | TYR | B | 393 | 35.894 | 36.786 | 66.218 | 1.00 | 54.75 | C | |
| ATOM | 6797 | CB | TYR | B | 393 | 36.452 | 35.416 | 65.860 | 1.00 | 54.46 | C | |
| ATOM | 6800 | CG | TYR | B | 393 | 36.644 | 34.571 | 67.068 | 1.00 | 54.65 | C | |
| ATOM | 6801 | CD1 | TYR | B | 393 | 35.581 | 33.863 | 67.614 | 1.00 | 53.91 | C | |
| ATOM | 6803 | CE1 | TYR | B | 393 | 35.748 | 33.090 | 68.769 | 1.00 | 55.30 | C | |
| ATOM | 6805 | CZ | TYR | B | 393 | 36.996 | 33.055 | 69.405 | 1.00 | 55.84 | C | |
| ATOM | 6806 | OH | TYR | B | 393 | 37.172 | 32.279 | 70.513 | 1.00 | 56.63 | O | |
| ATOM | 6808 | CE2 | TYR | B | 393 | 38.073 | 33.756 | 68.890 | 1.00 | 55.57 | C | |
| ATOM | 6810 | CD2 | TYR | B | 393 | 37.889 | 34.524 | 67.726 | 1.00 | 55.87 | C | |
| ATOM | 6812 | C | TYR | B | 393 | 36.901 | 37.602 | 67.027 | 1.00 | 54.29 | C | |
| ATOM | 6813 | O | TYR | B | 393 | 37.869 | 38.065 | 66.453 | 1.00 | 54.77 | O | |
| ATOM | 6815 | N | THR | B | 394 | 36.686 | 37.792 | 68.332 | 1.00 | 54.39 | N | |
| ATOM | 6816 | CA | THR | B | 394 | 37.691 | 38.462 | 69.188 | 1.00 | 54.91 | C | |
| ATOM | 6818 | CB | THR | B | 394 | 37.208 | 39.849 | 69.686 | 1.00 | 54.76 | C | |
| ATOM | 6820 | OG1 | THR | B | 394 | 37.009 | 40.709 | 68.562 | 1.00 | 56.00 | O | |
| ATOM | 6822 | CG2 | THR | B | 394 | 38.295 | 40.629 | 70.406 | 1.00 | 54.43 | C | |
| ATOM | 6826 | C | THR | B | 394 | 38.206 | 37.560 | 70.324 | 1.00 | 55.16 | C | |
| ATOM | 6827 | O | THR | B | 394 | 37.426 | 37.024 | 71.117 | 1.00 | 55.43 | O | |
| ATOM | 6829 | N | ALA | B | 395 | 39.535 | 37.437 | 70.378 | 1.00 | 55.30 | N | |
| ATOM | 6830 | CA | ALA | B | 395 | 40.227 | 36.603 | 71.326 | 1.00 | 55.97 | C | |
| ATOM | 6832 | CB | ALA | B | 395 | 41.565 | 36.166 | 70.749 | 1.00 | 56.01 | C | |
| ATOM | 6836 | C | ALA | B | 395 | 40.466 | 37.359 | 72.597 | 1.00 | 57.10 | C | |
| ATOM | 6837 | O | ALA | B | 395 | 40.604 | 38.590 | 72.571 | 1.00 | 57.98 | O | |
| ATOM | 6839 | N | HIS | B | 396 | 40.555 | 36.622 | 73.705 | 1.00 | 57.90 | N | |
| ATOM | 6840 | CA | HIS | B | 396 | 41.182 | 37.101 | 74.953 | 1.00 | 58.76 | C | |
| ATOM | 6842 | CB | HIS | B | 396 | 41.481 | 35.912 | 75.896 | 1.00 | 59.30 | C | |
| ATOM | 6845 | CG | HIS | B | 396 | 41.427 | 36.276 | 77.338 | 1.00 | 61.44 | C | |
| ATOM | 6846 | ND1 | HIS | B | 396 | 42.542 | 36.646 | 78.058 | 1.00 | 62.79 | N | |
| ATOM | 6848 | CE1 | HIS | B | 396 | 42.186 | 36.981 | 79.286 | 1.00 | 64.22 | C | |
| ATOM | 6850 | NE2 | HIS | B | 396 | 40.872 | 36.857 | 79.384 | 1.00 | 66.20 | N | |
| ATOM | 6852 | CD2 | HIS | B | 396 | 40.373 | 36.417 | 78.177 | 1.00 | 64.67 | C | |
| ATOM | 6854 | C | HIS | B | 396 | 42.490 | 37.907 | 74.758 | 1.00 | 58.74 | C | |
| ATOM | 6855 | O | HIS | B | 396 | 43.220 | 37.691 | 73.779 | 1.00 | 58.79 | O | |
| ATOM | 6857 | N | ALA | B | 397 | 42.767 | 38.848 | 75.670 | 1.00 | 58.96 | N | |
| ATOM | 6858 | CA | ALA | B | 397 | 44.137 | 39.420 | 75.810 | 1.00 | 58.93 | C | |
| ATOM | 6860 | CB | ALA | B | 397 | 44.137 | 40.701 | 76.581 | 1.00 | 58.43 | C | |
| ATOM | 6864 | C | ALA | B | 397 | 45.053 | 38.376 | 76.479 | 1.00 | 59.16 | C | |
| ATOM | 6865 | O | ALA | B | 397 | 44.610 | 37.341 | 76.984 | 1.00 | 58.04 | O | |
| ATOM | 6867 | N | GLY | B | 398 | 46.352 | 38.583 | 76.393 | 1.00 | 60.00 | N | |
| ATOM | 6868 | CA | GLY | B | 398 | 47.292 | 37.534 | 76.813 | 1.00 | 60.70 | C | |
| ATOM | 6871 | C | GLY | B | 398 | 47.303 | 36.267 | 75.966 | 1.00 | 60.73 | C | |
| ATOM | 6872 | O | GLY | B | 398 | 48.161 | 35.418 | 76.176 | 1.00 | 60.77 | O | |
| ATOM | 6874 | N | ALA | B | 399 | 46.328 | 36.105 | 75.060 | 1.00 | 60.88 | N | |
| ATOM | 6875 | CA | ALA | B | 399 | 46.495 | 35.287 | 73.858 | 1.00 | 60.56 | C | |
| ATOM | 6877 | CB | ALA | B | 399 | 45.196 | 35.173 | 73.074 | 1.00 | 60.48 | C | |
| ATOM | 6881 | C | ALA | B | 399 | 47.528 | 35.999 | 73.026 | 1.00 | 59.93 | C | |
| ATOM | 6882 | O | ALA | B | 399 | 47.247 | 37.080 | 72.529 | 1.00 | 60.40 | O | |
| ATOM | 6884 | N | LYS | B | 400 | 48.727 | 35.415 | 72.942 | 1.00 | 59.18 | N | |
| ATOM | 6885 | CA | LYS | B | 400 | 49.809 | 35.905 | 72.085 | 1.00 | 58.43 | C | |
| ATOM | 6887 | CB | LYS | B | 400 | 51.172 | 35.723 | 72.780 | 1.00 | 59.26 | C | |
| ATOM | 6890 | CG | LYS | B | 400 | 51.437 | 36.689 | 74.000 | 1.00 | 60.73 | C | |
| ATOM | 6893 | CD | LYS | B | 400 | 51.132 | 38.147 | 73.671 | 1.00 | 61.59 | C | |
| ATOM | 6896 | CE | LYS | B | 400 | 51.857 | 39.114 | 74.622 | 1.00 | 63.68 | C | |
| ATOM | 6899 | NZ | LYS | B | 400 | 51.721 | 40.562 | 74.159 | 1.00 | 63.82 | N | |
| ATOM | 6903 | C | LYS | B | 400 | 49.782 | 35.191 | 70.734 | 1.00 | 57.05 | C | |
| ATOM | 6904 | O | LYS | B | 400 | 49.522 | 33.996 | 70.665 | 1.00 | 56.84 | O | |
| ATOM | 6906 | N | PHE | B | 401 | 50.031 | 35.941 | 69.660 | 1.00 | 55.67 | N | |
| ATOM | 6907 | CA | PHE | B | 401 | 50.090 | 35.403 | 68.308 | 1.00 | 54.70 | C | |
| ATOM | 6909 | CB | PHE | B | 401 | 48.921 | 35.953 | 67.524 | 1.00 | 55.18 | C | |
| ATOM | 6912 | CG | PHE | B | 401 | 47.620 | 35.512 | 68.066 | 1.00 | 55.72 | C | |
| ATOM | 6913 | CD1 | PHE | B | 401 | 47.185 | 34.172 | 67.850 | 1.00 | 57.74 | C | |
| ATOM | 6915 | CE1 | PHE | B | 401 | 45.937 | 33.698 | 68.383 | 1.00 | 57.37 | C | |
| ATOM | 6917 | CZ | PHE | B | 401 | 45.134 | 34.587 | 69.173 | 1.00 | 57.21 | C | |
| ATOM | 6919 | CE2 | PHE B | 401 | 45.587 | 35.950 | 69.400 | 1.00 | 57.49 | C | | |
| ATOM | 6921 | CD2 | PHE | B | 401 | 46.829 | 36.391 | 68.841 | 1.00 | 56.67 | C | |
| ATOM | 6923 | C | PHE | B | 401 | 51.431 | 35.730 | 67.632 | 1.00 | 54.26 | C | |
| ATOM | 6924 | O | PHE | B | 401 | 52.051 | 36.739 | 68.018 | 1.00 | 54.77 | O | |
| ATOM | 6926 | N | PRO | B | 402 | 51.891 | 34.909 | 66.659 | 1.00 | 52.89 | N | |
| ATOM | 6927 | CA | PRO | B | 402 | 53.141 | 35.165 | 65.939 | 1.00 | 52.59 | C | |
| ATOM | 6929 | CB | PRO | B | 402 | 53.244 | 33.985 | 64.980 | 1.00 | 52.04 | C | |
| ATOM | 6932 | CG | PRO | B | 402 | 51.915 | 33.574 | 64.804 | 1.00 | 52.25 | C | |
| ATOM | 6935 | CD | PRO | B | 402 | 51.306 | 33.666 | 66.162 | 1.00 | 52.70 | C | |
| ATOM | 6938 | C | PRO | B | 402 | 53.158 | 36.473 | 65.167 | 1.00 | 51.28 | C | |
| ATOM | 6939 | O | PRO | B | 402 | 52.410 | 36.655 | 64.223 | 1.00 | 50.93 | O | |
| ATOM | 6940 | N | ILE | B | 403 | 54.068 | 37.347 | 65.564 | 1.00 | 50.49 | N | |
| ATOM | 6941 | CA | ILE | B | 403 | 54.118 | 38.711 | 65.066 | 1.00 | 49.71 | C | |
| ATOM | 6943 | CB | ILE | B | 403 | 55.214 | 39.513 | 65.779 | 1.00 | 49.57 | C | |
| ATOM | 6945 | CG1 | ILE | B | 403 | 54.852 | 39.605 | 67.274 | 1.00 | 48.53 | C | |
| ATOM | 6948 | CD1 | ILE | B | 403 | 55.784 | 40.361 | 68.113 | 1.00 | 48.55 | C | |
| ATOM | 6952 | CG2 | ILE | B | 403 | 55.381 | 40.942 | 65.131 | 1.00 | 50.49 | C | |
| ATOM | 6956 | C | ILE | B | 403 | 54.230 | 38.792 | 63.554 | 1.00 | 49.87 | C | |
| ATOM | 6957 | O | ILE | B | 403 | 53.558 | 39.637 | 62.925 | 1.00 | 51.16 | O | |
| ATOM | 6959 | N | LYS | B | 404 | 55.039 | 37.919 | 62.963 | 1.00 | 49.88 | N | |
| ATOM | 6960 | CA | LYS | B | 404 | 55.349 | 38.027 | 61.518 | 1.00 | 49.82 | C | |
| ATOM | 6962 | CB | LYS | B | 404 | 56.691 | 37.389 | 61.196 | 1.00 | 50.80 | C | |
| ATOM | 6965 | CG | LYS | B | 404 | 57.835 | 38.174 | 61.804 | 1.00 | 53.31 | C | |
| ATOM | 6968 | CD | LYS | B | 404 | 59.118 | 37.480 | 61.460 | 1.00 | 58.47 | C | |
| ATOM | 6971 | CE | LYS | B | 404 | 60.368 | 38.173 | 62.014 | 1.00 | 58.71 | C | |
| ATOM | 6974 | NZ | LYS | B | 404 | 61.326 | 37.028 | 62.268 | 1.00 | 63.21 | N | |
| ATOM | 6978 | C | LYS | B | 404 | 54.256 | 37.540 | 60.584 | 1.00 | 47.84 | C | |
| ATOM | 6979 | O | LYS | B | 404 | 54.318 | 37.752 | 59.415 | 1.00 | 47.75 | O | |
| ATOM | 6981 | N | TRP | B | 405 | 53.258 | 36.895 | 61.114 | 1.00 | 46.55 | N | |
| ATOM | 6982 | CA | TRP | B | 405 | 52.202 | 36.345 | 60.315 | 1.00 | 46.04 | C | |
| ATOM | 6984 | CB | TRP | B | 405 | 52.140 | 34.829 | 60.550 | 1.00 | 45.86 | C | |
| ATOM | 6987 | CG | TRP | B | 405 | 53.214 | 34.119 | 59.790 | 1.00 | 44.84 | C | |
| ATOM | 6988 | CD1 | TRP | B | 405 | 53.122 | 33.677 | 58.527 | 1.00 | 45.06 | C | |
| ATOM | 6990 | NE1 | TRP | B | 405 | 54.284 | 33.051 | 58.141 | 1.00 | 45.50 | N | |
| ATOM | 6992 | CE2 | TRP | B | 405 | 55.185 | 33.142 | 59.157 | 1.00 | 44.92 | C | |
| ATOM | 6993 | CD2 | TRP | B | 405 | 54.535 | 33.832 | 60.219 | 1.00 | 44.66 | C | |
| ATOM | 6994 | CE3 | TRP | B | 405 | 55.246 | 34.046 | 61.408 | 1.00 | 46.33 | C | |
| ATOM | 6996 | CZ3 | TRP | B | 405 | 56.603 | 33.566 | 61.487 | 1.00 | 45.72 | C | |
| ATOM | 6998 | CH2 | TRP | B | 405 | 57.203 | 32.901 | 60.398 | 1.00 | 43.78 | C | |
| ATOM | 7000 | CZ2 | TRP | B | 405 | 56.521 | 32.691 | 59.231 | 1.00 | 43.41 | C | |
| ATOM | 7002 | C | TRP | B | 405 | 50.885 | 36.969 | 60.586 | 1.00 | 45.54 | C | |
| ATOM | 7003 | O | TRP | B | 405 | 50.030 | 36.874 | 59.728 | 1.00 | 46.42 | O | |
| ATOM | 7005 | N | THR | B | 406 | 50.726 | 37.619 | 61.740 | 1.00 | 45.08 | N | |
| ATOM | 7006 | CA | THR | B | 406 | 49.462 | 38.119 | 62.190 | 1.00 | 44.69 | C | |
| ATOM | 7008 | CB | THR | B | 406 | 49.469 | 38.239 | 63.669 | 1.00 | 44.33 | C | |
| ATOM | 7010 | OG1 | THR | B | 406 | 49.883 | 37.006 | 64.260 | 1.00 | 46.89 | O | |
| ATOM | 7012 | CG2 | THR | B | 406 | 48.074 | 38.480 | 64.206 | 1.00 | 44.14 | C | |
| ATOM | 7016 | C | THR | B | 406 | 49.297 | 39.492 | 61.646 | 1.00 | 45.36 | C | |
| ATOM | 7017 | O | THR | B | 406 | 50.269 | 40.294 | 61.610 | 1.00 | 46.48 | O | |
| ATOM | 7019 | N | ALA | B | 407 | 48.038 | 39.821 | 61.347 | 1.00 | 45.45 | N | |
| ATOM | 7020 | CA | ALA | B | 407 | 47.699 | 41.135 | 60.814 | 1.00 | 44.77 | C | |
| ATOM | 7022 | CB | ALA | B | 407 | 46.327 | 41.129 | 60.222 | 1.00 | 44.93 | C | |
| ATOM | 7026 | C | ALA | B | 407 | 47.770 | 42.114 | 61.939 | 1.00 | 44.66 | C | |
| ATOM | 7027 | O | ALA | B | 407 | 47.586 | 41.691 | 63.045 | 1.00 | 44.40 | O | |
| ATOM | 7029 | N | PRO | B | 408 | 48.028 | 43.404 | 61.658 | 1.00 | 45.22 | N | |
| ATOM | 7030 | CA | PRO | B | 408 | 48.116 | 44.459 | 62.644 | 1.00 | 44.88 | C | |
| ATOM | 7032 | CB | PRO | B | 408 | 48.069 | 45.723 | 61.779 | 1.00 | 44.93 | C | |
| ATOM | 7035 | CG | PRO | B | 408 | 48.610 | 45.368 | 60.579 | 1.00 | 44.95 | C | |
| ATOM | 7038 | CD | PRO | B | 408 | 48.293 | 43.958 | 60.327 | 1.00 | 45.57 | C | |
| ATOM | 7041 | C | PRO | B | 408 | 46.998 | 44.512 | 63.659 | 1.00 | 45.59 | C | |
| ATOM | 7042 | O | PRO | B | 408 | 47.247 | 44.430 | 64.864 | 1.00 | 45.08 | O | |
| ATOM | 7043 | N | GLU | B | 409 | 45.771 | 44.635 | 63.170 | 1.00 | 46.70 | N | |
| ATOM | 7044 | CA | GLU | B | 409 | 44.604 | 44.803 | 64.043 | 1.00 | 47.26 | C | |
| ATOM | 7046 | CB | GLU | B | 409 | 43.363 | 45.231 | 63.255 | 1.00 | 46.76 | C | |
| ATOM | 7049 | CG | GLU | B | 409 | 42.746 | 44.214 | 62.324 | 1.00 | 47.23 | C | |
| ATOM | 7052 | CD | GLU | B | 409 | 43.528 | 43.953 | 61.050 | 1.00 | 50.93 | C | |
| ATOM | 7053 | OE1 | GLU | B | 409 | 44.494 | 44.705 | 60.771 | 1.00 | 54.85 | O | |
| ATOM | 7054 | OE2 | GLU | B | 409 | 43.193 | 42.970 | 60.336 | 1.00 | 51.60 | O | |
| ATOM | 7055 | C | GLU | B | 409 | 44.359 | 43.532 | 64.896 | 1.00 | 48.10 | C | |
| ATOM | 7056 | O | GLU | B | 409 | 43.832 | 43.628 | 66.033 | 1.00 | 47.56 | O | |
| ATOM | 7058 | N | SER | B | 410 | 44.729 | 42.365 | 64.339 | 1.00 | 48.23 | N | |
| ATOM | 7059 | CA | SER | B | 410 | 44.772 | 41.165 | 65.126 | 1.00 | 49.12 | C | |
| ATOM | 7061 | CB | SER | B | 410 | 44.946 | 39.959 | 64.260 | 1.00 | 49.42 | C | |
| ATOM | 7064 | OG | SER | B | 410 | 43.798 | 39.792 | 63.472 | 1.00 | 52.68 | O | |
| ATOM | 7066 | C | SER | B | 410 | 45.849 | 41.215 | 66.202 | 1.00 | 49.38 | C | |
| ATOM | 7067 | O | SER | B | 410 | 45.574 | 40.776 | 67.308 | 1.00 | 50.23 | O | |
| ATOM | 7069 | N | LEU | B | 411 | 47.045 | 41.742 | 65.902 | 1.00 | 49.54 | N | |
| ATOM | 7070 | CA | LEU | B | 411 | 48.091 | 42.022 | 66.943 | 1.00 | 49.72 | C | |
| ATOM | 7072 | CB | LEU | B | 411 | 49.476 | 42.437 | 66.336 | 1.00 | 49.50 | C | |
| ATOM | 7075 | CG | LEU | B | 411 | 50.296 | 41.378 | 65.561 | 1.00 | 51.21 | C | |
| ATOM | 7077 | CD1 | LEU | B | 411 | 51.634 | 41.935 | 65.153 | 1.00 | 51.19 | C | |
| ATOM | 7081 | CD2 | LEU | B | 411 | 50.523 | 40.053 | 66.360 | 1.00 | 52.38 | C | |
| ATOM | 7085 | C | LEU | B | 411 | 47.679 | 43.078 | 67.992 | 1.00 | 49.70 | C | |
| ATOM | 7086 | O | LEU | B | 411 | 48.017 | 42.935 | 69.152 | 1.00 | 49.53 | O | |
| ATOM | 7088 | N | ALA | B | 412 | 46.966 | 44.117 | 67.571 | 1.00 | 49.85 | N | |
| ATOM | 7089 | CA | ALA | B | 412 | 46.724 | 45.306 | 68.383 | 1.00 | 50.42 | C | |
| ATOM | 7091 | CB | ALA | B | 412 | 46.709 | 46.573 | 67.487 | 1.00 | 49.98 | C | |
| ATOM | 7095 | C | ALA | B | 412 | 45.431 | 45.222 | 69.209 | 1.00 | 51.15 | C | |
| ATOM | 7096 | O | ALA | B | 412 | 45.399 | 45.719 | 70.328 | 1.00 | 52.64 | O | |
| ATOM | 7098 | N | TYR | B | 413 | 44.374 | 44.622 | 68.662 | 1.00 | 51.39 | N | |
| ATOM | 7099 | CA | TYR | B | 413 | 43.081 | 44.502 | 69.316 | 1.00 | 51.21 | C | |
| ATOM | 7101 | CB | TYR | B | 413 | 42.055 | 45.369 | 68.581 | 1.00 | 52.01 | C | |
| ATOM | 7104 | CG | TYR | B | 413 | 42.568 | 46.726 | 68.223 | 1.00 | 53.18 | C | |
| ATOM | 7105 | CD1 | TYR | B | 413 | 42.447 | 47.793 | 69.110 | 1.00 | 54.41 | C | |
| ATOM | 7107 | CE1 | TYR | B | 413 | 42.942 | 49.062 | 68.786 | 1.00 | 53.23 | C | |
| ATOM | 7109 | CZ | TYR | B | 413 | 43.573 | 49.266 | 67.575 | 1.00 | 53.15 | C | |
| ATOM | 7110 | OH | TYR | B | 413 | 44.061 | 50.514 | 67.270 | 1.00 | 54.57 | O | |
| ATOM | 7112 | CE2 | TYR | B 413 | 43.719 | 48.219 | 66.676 | 1.00 | 54.14 | C | | |
| ATOM | 7114 | CD2 | TYR | B | 413 | 43.209 | 46.953 | 67.001 | 1.00 | 53.77 | C | |
| ATOM | 7116 | C | TYR | B | 413 | 42.560 | 43.063 | 69.370 | 1.00 | 51.19 | C | |
| ATOM | 7117 | O | TYR | B | 413 | 41.400 | 42.883 | 69.706 | 1.00 | 51.03 | O | |
| ATOM | 7119 | N | ASN | B | 414 | 43.365 | 42.045 | 69.043 | 1.00 | 51.30 | N | |
| ATOM | 7120 | CA | ASN | B | 414 | 42.890 | 40.646 | 69.001 | 1.00 | 51.62 | C | |
| ATOM | 7122 | CB | ASN | B | 414 | 42.772 | 40.090 | 70.441 | 1.00 | 52.08 | C | |
| ATOM | 7125 | CG | ASN | B | 414 | 44.121 | 39.975 | 71.137 | 1.00 | 52.36 | C | |
| ATOM | 7126 | OD1 | ASN | B | 414 | 44.768 | 38.937 | 71.079 | 1.00 | 49.91 | O | |
| ATOM | 7127 | ND2 | ASN | B | 414 | 44.563 | 41.065 | 71.769 | 1.00 | 53.70 | N | |
| ATOM | 7130 | C | ASN | B | 414 | 41.571 | 40.456 | 68.211 | 1.00 | 51.85 | C | |
| ATOM | 7131 | O | ASN | B | 414 | 40.753 | 39.628 | 68.560 | 1.00 | 52.68 | O | |
| ATOM | 7133 | N | LYS | B | 415 | 41.373 | 41.247 | 67.155 | 1.00 | 51.82 | N | |
| ATOM | 7134 | CA | LYS | B | 415 | 40.178 | 41.186 | 66.287 | 1.00 | 51.60 | C | |
| ATOM | 7136 | CB | LYS | B | 415 | 39.743 | 42.600 | 65.801 | 1.00 | 52.48 | C | |
| ATOM | 7139 | CG | LYS | B | 415 | 38.839 | 43.413 | 66.764 | 1.00 | 53.82 | C | |
| ATOM | 7142 | CD | LYS | B | 415 | 38.122 | 44.554 | 66.037 | 1.00 | 53.50 | C | |
| ATOM | 7145 | CE | LYS | B | 415 | 37.629 | 45.612 | 67.050 | 1.00 | 56.22 | C | |
| ATOM | 7148 | NZ | LYS | B | 415 | 37.256 | 46.951 | 66.440 | 1.00 | 56.46 | N | |
| ATOM | 7152 | C | LYS | B | 415 | 40.565 | 40.368 | 65.075 | 1.00 | 49.64 | C | |
| ATOM | 7153 | O | LYS | B | 415 | 41.317 | 40.844 | 64.231 | 1.00 | 49.81 | O | |
| ATOM | 7155 | N | PHE | B | 416 | 40.056 | 39.156 | 64.987 | 1.00 | 47.61 | N | |
| ATOM | 7156 | CA | PHE | B | 416 | 40.281 | 38.314 | 63.824 | 1.00 | 46.43 | C | |
| ATOM | 7158 | CB | PHE | B | 416 | 40.632 | 36.907 | 64.272 | 1.00 | 45.58 | C | |
| ATOM | 7161 | CG | PHE | B | 416 | 41.909 | 36.847 | 65.011 | 1.00 | 44.03 | C | |
| ATOM | 7162 | CD1 | PHE | B | 416 | 41.941 | 37.043 | 66.388 | 1.00 | 42.39 | C | |
| ATOM | 7164 | CE1 | PHE | B | 416 | 43.128 | 36.977 | 67.054 | 1.00 | 41.98 | C | |
| ATOM | 7166 | CZ | PHE | B | 416 | 44.331 | 36.777 | 66.314 | 1.00 | 41.32 | C | |
| ATOM | 7168 | CE2 | PHE | B | 416 | 44.287 | 36.628 | 64.964 | 1.00 | 39.81 | C | |
| ATOM | 7170 | CD2 | PHE | B | 416 | 43.092 | 36.651 | 64.323 | 1.00 | 41.51 | C | |
| ATOM | 7172 | C | PHE | B | 416 | 39.075 | 38.336 | 62.869 | 1.00 | 46.31 | C | |
| ATOM | 7173 | O | PHE | B | 416 | 37.962 | 38.718 | 63.242 | 1.00 | 45.52 | O | |
| ATOM | 7175 | N | SER | B | 417 | 39.340 | 37.972 | 61.620 | 1.00 | 46.14 | N | |
| ATOM | 7176 | CA | SER | B | 417 | 38.333 | 38.003 | 60.577 | 1.00 | 46.57 | C | |
| ATOM | 7178 | CB | SER | B | 417 | 38.034 | 39.480 | 60.174 | 1.00 | 46.06 | C | |
| ATOM | 7181 | OG | SER | B | 417 | 38.993 | 40.002 | 59.267 | 1.00 | 45.60 | O | |
| ATOM | 7183 | C | SER | B | 417 | 38.778 | 37.141 | 59.368 | 1.00 | 46.88 | C | |
| ATOM | 7184 | O | SER | B | 417 | 39.834 | 36.551 | 59.367 | 1.00 | 47.28 | O | |
| ATOM | 7186 | N | ILE | B | 418 | 37.964 | 37.077 | 58.335 | 1.00 | 47.08 | N | |
| ATOM | 7187 | CA | ILE | B | 418 | 38.432 | 36.562 | 57.055 | 1.00 | 47.50 | C | |
| ATOM | 7189 | CB | ILE | B | 418 | 37.235 | 36.318 | 56.105 | 1.00 | 46.99 | C | |
| ATOM | 7191 | CG | ILE | B | 418 | 36.518 | 35.059 | 56.641 | 1.00 | 49.87 | C | |
| ATOM | 7194 | CD1 | ILE | B | 418 | 35.104 | 34.786 | 56.134 | 1.00 | 50.68 | C | |
| ATOM | 7198 | CG2 | ILE | B | 418 | 37.704 | 36.060 | 54.691 | 1.00 | 46.24 | C | |
| ATOM | 7202 | C | ILE | B | 418 | 39.552 | 37.430 | 56.486 | 1.00 | 46.42 | C | |
| ATOM | 7203 | O | ILE | B | 418 | 40.550 | 36.903 | 56.018 | 1.00 | 48.00 | O | |
| ATOM | 7205 | N | LYS | B | 419 | 39.442 | 38.741 | 56.620 | 1.00 | 45.30 | N | |
| ATOM | 7206 | CA | LYS | B | 419 | 40.518 | 39.632 | 56.178 | 1.00 | 44.18 | C | |
| ATOM | 7208 | CB | LYS | B | 419 | 40.096 | 41.082 | 56.210 | 1.00 | 43.26 | C | |
| ATOM | 7211 | CG | LYS | B | 419 | 38.797 | 41.379 | 55.479 | 1.00 | 43.63 | C | |
| ATOM | 7214 | CD | LYS | B | 419 | 38.754 | 40.859 | 54.036 | 1.00 | 43.15 | C | |
| ATOM | 7217 | CE | LYS | B | 419 | 37.933 | 41.751 | 53.104 | 1.00 | 41.60 | C | |
| ATOM | 7220 | NZ | LYS | B | 419 | 38.179 | 41.398 | 51.699 | 1.00 | 39.80 | N | |
| ATOM | 7224 | C | LYS | B | 419 | 41.813 | 39.447 | 56.972 | 1.00 | 43.94 | C | |
| ATOM | 7225 | O | LYS | B | 419 | 42.877 | 39.653 | 56.415 | 1.00 | 43.27 | O | |
| ATOM | 7227 | N | SER | B | 420 | 41.754 | 39.024 | 58.235 | 1.00 | 44.25 | N | |
| ATOM | 7228 | CA | SER | B | 420 | 43.013 | 38.800 | 58.970 | 1.00 | 45.21 | C | |
| ATOM | 7230 | CB | SER | B | 420 | 42.827 | 38.644 | 60.481 | 1.00 | 44.92 | C | |
| ATOM | 7233 | OG | SER | B | 420 | 42.397 | 37.343 | 60.798 | 1.00 | 47.44 | O | |
| ATOM | 7235 | C | SER | B | 420 | 43.757 | 37.624 | 58.330 | 1.00 | 45.51 | C | |
| ATOM | 7236 | O | SER | B | 420 | 44.928 | 37.772 | 58.019 | 1.00 | 46.91 | O | |
| ATOM | 7238 | N | ASP | B | 421 | 43.066 | 36.520 | 58.045 | 1.00 | 45.42 | N | |
| ATOM | 7239 | CA | ASP | B | 421 | 43.659 | 35.395 | 57.297 | 1.00 | 45.45 | C | |
| ATOM | 7241 | CB | ASP | B | 421 | 42.666 | 34.235 | 57.143 | 1.00 | 46.29 | C | |
| ATOM | 7244 | CG | ASP | B | 421 | 42.386 | 33.503 | 58.463 | 1.00 | 49.26 | C | |
| ATOM | 7245 | OD1 | ASP | B | 421 | 42.682 | 34.047 | 59.592 | 1.00 | 53.74 | O | |
| ATOM | 7246 | OD2 | ASP | B | 421 | 41.798 | 32.398 | 58.445 | 1.00 | 49.93 | O | |
| ATOM | 7247 | C | ASP | B | 421 | 44.181 | 35.753 | 55.913 | 1.00 | 44.47 | C | |
| ATOM | 7248 | O | ASP | B | 421 | 45.185 | 35.198 | 55.478 | 1.00 | 45.25 | O | |
| ATOM | 7250 | N | VAL | B | 422 | 43.513 | 36.649 | 55.209 | 1.00 | 42.85 | N | |
| ATOM | 7251 | CA | VAL | B | 422 | 44.069 | 37.171 | 53.948 | 1.00 | 42.15 | C | |
| ATOM | 7253 | CB | VAL | B | 422 | 42.993 | 38.103 | 53.269 | 1.00 | 42.62 | C | |
| ATOM | 7255 | CG1 | VAL | B | 422 | 43.566 | 39.073 | 52.223 | 1.00 | 43.05 | C | |
| ATOM | 7259 | CG2 | VAL | B | 422 | 41.884 | 37.257 | 52.682 | 1.00 | 41.64 | C | |
| ATOM | 7263 | C | VAL | B | 422 | 45.511 | 37.823 | 54.122 | 1.00 | 41.82 | C | |
| ATOM | 7264 | O | VAL | B | 422 | 46.427 | 37.657 | 53.260 | 1.00 | 41.08 | O | |
| ATOM | 7266 | N | TRP | B | 423 | 45.719 | 38.520 | 55.239 | 1.00 | 40.82 | N | |
| ATOM | 7267 | CA | TRP | B | 423 | 47.052 | 39.077 | 55.542 | 1.00 | 40.53 | C | |
| ATOM | 7269 | CB | TRP | B | 423 | 47.053 | 39.849 | 56.846 | 1.00 | 39.80 | C | |
| ATOM | 7272 | CG | TRP | B | 423 | 48.333 | 40.436 | 57.171 | 1.00 | 39.83 | C | |
| ATOM | 7273 | CD1 | TRP | B | 423 | 49.423 | 39.796 | 57.666 | 1.00 | 40.26 | C | |
| ATOM | 7275 | NE1 | TRP | B | 423 | 50.440 | 40.690 | 57.889 | 1.00 | 40.52 | N | |
| ATOM | 7277 | CE2 | TRP | B | 423 | 50.008 | 41.941 | 57.569 | 1.00 | 39.09 | C | |
| ATOM | 7278 | CD2 | TRP | B | 423 | 48.674 | 41.815 | 57.119 | 1.00 | 41.19 | C | |
| ATOM | 7279 | CE3 | TRP | B | 423 | 47.978 | 42.978 | 56.762 | 1.00 | 41.01 | C | |
| ATOM | 7281 | CZ3 | TRP | B | 423 | 48.656 | 44.231 | 56.820 | 1.00 | 40.90 | C | |
| ATOM | 7283 | CH2 | TRP | B | 423 | 49.996 | 44.310 | 57.244 | 1.00 | 40.22 | C | |
| ATOM | 7285 | CZ2 | TRP | B | 423 | 50.682 | 43.170 | 57.631 | 1.00 | 40.16 | C | |
| ATOM | 7287 | C | TRP | B | 423 | 48.086 | 37.966 | 55.600 | 1.00 | 39.71 | C | |
| ATOM | 7288 | O | TRP | B | 423 | 49.107 | 38.043 | 54.933 | 1.00 | 38.99 | O | |
| ATOM | 7290 | N | ALA | B | 424 | 47.793 | 36.971 | 56.431 | 1.00 | 39.53 | N | |
| ATOM | 7291 | CA | ALA | B | 424 | 48.636 | 35.802 | 56.644 | 1.00 | 39.67 | C | |
| ATOM | 7293 | CB | ALA | B | 424 | 47.987 | 34.810 | 57.591 | 1.00 | 39.62 | C | |
| ATOM | 7297 | C | ALA | B | 424 | 48.884 | 35.121 | 55.322 | 1.00 | 39.93 | C | |
| ATOM | 7298 | O | ALA | B | 424 | 50.023 | 34.868 | 54.959 | 1.00 | 39.41 | O | |
| ATOM | 7300 | N | PHE | B | 425 | 47.817 | 34.893 | 54.569 | 1.00 | 40.73 | N | |
| ATOM | 7301 | CA | PHE | B | 425 | 47.956 | 34.337 | 53.246 | 1.00 | 41.46 | C | |
| ATOM | 7303 | CB | PHE | B | 425 | 46.618 | 34.271 | 52.520 | 1.00 | 41.19 | C | |
| ATOM | 7306 | CG | PHE | B | 425 | 46.756 | 33.764 | 51.148 | 1.00 | 42.09 | C | |
| ATOM | 7307 | CD1 | PHE | B | 425 | 46.779 | 32.342 | 50.915 | 1.00 | 41.45 | C | |
| ATOM | 7309 | CE1 | PHE | B | 425 | 46.987 | 31.816 | 49.655 | 1.00 | 38.78 | C | |
| ATOM | 7311 | CZ | PHE | B | 425 | 47.195 | 32.712 | 48.583 | 1.00 | 40.92 | C | |
| ATOM | 7313 | CE2 | PHE | B | 425 | 47.229 | 34.167 | 48.814 | 1.00 | 41.74 | C | |
| ATOM | 7315 | CD2 | PHE | B | 425 | 47.012 | 34.670 | 50.092 | 1.00 | 39.72 | C | |
| ATOM | 7317 | C | PHE | B | 425 | 49.006 | 35.123 | 52.451 | 1.00 | 41.98 | C | |
| ATOM | 7318 | O | PHE | B | 425 | 49.854 | 34.516 | 51.846 | 1.00 | 42.33 | O | |
| ATOM | 7320 | N | GLY | B | 426 | 48.955 | 36.469 | 52.503 | 1.00 | 43.52 | N | |
| ATOM | 7321 | CA | GLY | B | 426 | 50.014 | 37.371 | 51.959 | 1.00 | 42.94 | C | |
| ATOM | 7324 | C | GLY | B | 426 | 51.440 | 36.975 | 52.290 | 1.00 | 43.22 | C | |
| ATOM | 7325 | O | GLY | B | 426 | 52.302 | 36.952 | 51.416 | 1.00 | 43.25 | O | |
| ATOM | 7327 | N | VAL | B | 427 | 51.660 | 36.623 | 53.554 | 1.00 | 43.45 | N | |
| ATOM | 7328 | CA | VAL | B | 427 | 52.980 | 36.205 | 54.077 | 1.00 | 43.77 | C | |
| ATOM | 7330 | CB | VAL | B | 427 | 53.063 | 36.327 | 55.624 | 1.00 | 43.99 | C | |
| ATOM | 7332 | CG1 | VAL | B | 427 | 54.468 | 35.973 | 56.147 | 1.00 | 45.40 | C | |
| ATOM | 7336 | CG2 | VAL | B | 427 | 52.687 | 37.756 | 56.071 | 1.00 | 44.09 | C | |
| ATOM | 7340 | C | VAL | B | 427 | 53.298 | 34.771 | 53.670 | 1.00 | 43.37 | C | |
| ATOM | 7341 | O | VAL | B | 427 | 54.451 | 34.429 | 53.416 | 1.00 | 43.04 | O | |
| ATOM | 7343 | N | LEU | B | 428 | 52.278 | 33.942 | 53.600 | 1.00 | 43.10 | N | |
| ATOM | 7344 | CA | LEU | B | 428 | 52.428 | 32.630 | 53.060 | 1.00 | 43.80 | C | |
| ATOM | 7346 | CB | LEU | B | 428 | 51.119 | 31.860 | 53.084 | 1.00 | 43.17 | C | |
| ATOM | 7349 | CG | LEU | B | 428 | 51.174 | 30.429 | 52.564 | 1.00 | 45.01 | C | |
| ATOM | 7351 | CD1 | LEU | B | 428 | 52.486 | 29.666 | 52.813 | 1.00 | 46.52 | C | |
| ATOM | 7355 | CD2 | LEU | B | 428 | 50.029 | 29.630 | 53.231 | 1.00 | 45.52 | C | |
| ATOM | 7359 | C | LEU | B | 428 | 52.962 | 32.708 | 51.642 | 1.00 | 44.29 | C | |
| ATOM | 7360 | O | LEU | B | 428 | 53.845 | 31.915 | 51.263 | 1.00 | 45.65 | O | |
| ATOM | 7362 | N | LEU | B | 429 | 52.469 | 33.663 | 50.862 | 1.00 | 43.82 | N | |
| ATOM | 7363 | CA | LEU | B | 429 | 52.914 | 33.770 | 49.485 | 1.00 | 43.60 | C | |
| ATOM | 7365 | CB | LEU | B | 429 | 52.122 | 34.824 | 48.722 | 1.00 | 43.53 | C | |
| ATOM | 7368 | CG | LEU | B | 429 | 50.752 | 34.486 | 48.209 | 1.00 | 42.53 | C | |
| ATOM | 7370 | CD1 | LEU | B | 429 | 50.241 | 35.683 | 47.456 | 1.00 | 43.70 | C | |
| ATOM | 7374 | CD2 | LEU | B | 429 | 50.817 | 33.249 | 47.285 | 1.00 | 42.23 | C | |
| ATOM | 7378 | C | LEU | B | 429 | 54.376 | 34.135 | 49.422 | 1.00 | 44.23 | C | |
| ATOM | 7379 | O | LEU | B | 429 | 55.088 | 33.695 | 48.492 | 1.00 | 44.56 | O | |
| ATOM | 7381 | N | TRP | B | 430 | 54.790 | 34.963 | 50.392 | 1.00 | 44.50 | N | |
| ATOM | 7382 | CA | TRP | B | 430 | 56.149 | 35.466 | 50.486 | 1.00 | 44.13 | C | |
| ATOM | 7384 | CB | TRP | B | 430 | 56.278 | 36.578 | 51.556 | 1.00 | 45.43 | C | |
| ATOM | 7387 | CG | TRP | B | 430 | 57.602 | 37.287 | 51.497 | 1.00 | 45.49 | C | |
| ATOM | 7388 | CD1 | TRP | B | 430 | 57.918 | 38.322 | 50.703 | 1.00 | 46.10 | C | |
| ATOM | 7390 | NE1 | TRP | B | 430 | 59.233 | 38.684 | 50.878 | 1.00 | 46.68 | N | |
| ATOM | 7392 | CE2 | TRP | B | 430 | 59.805 | 37.841 | 51.787 | 1.00 | 45.24 | C | |
| ATOM | 7393 | CD2 | TRP | B | 430 | 58.795 | 36.939 | 52.206 | 1.00 | 45.96 | C | |
| ATOM | 7394 | CE3 | TRP | B | 430 | 59.116 | 35.952 | 53.166 | 1.00 | 46.57 | C | |
| ATOM | 7396 | CZ3 | TRP | B | 430 | 60.409 | 35.916 | 53.675 | 1.00 | 46.75 | C | |
| ATOM | 7398 | CH2 | TRP | B | 430 | 61.401 | 36.864 | 53.230 | 1.00 | 46.23 | C | |
| ATOM | 7400 | CZ2 | TRP | B | 430 | 61.105 | 37.823 | 52.298 | 1.00 | 45.00 | C | |
| ATOM | 7402 | C | TRP | B | 430 | 57.072 | 34.358 | 50.804 | 1.00 | 42.97 | C | |
| ATOM | 7403 | O | TRP | B | 430 | 58.144 | 34.331 | 50.280 | 1.00 | 41.66 | O | |
| ATOM | 7405 | N | GLU | B | 431 | 56.647 | 33.443 | 51.662 | 1.00 | 43.57 | N | |
| ATOM | 7406 | CA | GLU | B | 431 | 57.500 | 32.285 | 52.048 | 1.00 | 44.58 | C | |
| ATOM | 7408 | CB | GLU | B | 431 | 56.921 | 31.499 | 53.217 | 1.00 | 43.57 | C | |
| ATOM | 7411 | CG | GLU | B | 431 | 56.872 | 32.230 | 54.558 | 1.00 | 45.47 | C | |
| ATOM | 7414 | CD | GLU | B | 431 | 56.223 | 31.363 | 55.657 | 1.00 | 48.63 | C | |
| ATOM | 7415 | OE1 | GLU | B | 431 | 54.937 | 31.250 | 55.709 | 1.00 | 50.27 | O | |
| ATOM | 7416 | OE2 | GLU | B | 431 | 57.023 | 30.744 | 56.443 | 1.00 | 54.96 | O | |
| ATOM | 7417 | C | GLU | B | 431 | 57.667 | 31.347 | 50.884 | 1.00 | 44.43 | C | |
| ATOM | 7418 | O | GLU | B | 431 | 58.723 | 30.745 | 50.727 | 1.00 | 45.17 | O | |
| ATOM | 7420 | N | ILE | B | 432 | 56.613 | 31.213 | 50.076 | 1.00 | 44.85 | N | |
| ATOM | 7421 | CA | ILE | B | 432 | 56.627 | 30.325 | 48.900 | 1.00 | 44.73 | C | |
| ATOM | 7423 | CB | ILE | B | 432 | 55.170 | 30.162 | 48.341 | 1.00 | 44.71 | C | |
| ATOM | 7425 | CG1 | ILE | B | 432 | 54.386 | 29.291 | 49.294 | 1.00 | 44.20 | C | |
| ATOM | 7428 | CD1 | ILE | B | 432 | 52.932 | 29.328 | 49.080 | 1.00 | 46.03 | C | |
| ATOM | 7432 | CG2 | ILE | B | 432 | 55.139 | 29.575 | 46.912 | 1.00 | 44.97 | C | |
| ATOM | 7436 | C | ILE | B | 432 | 57.638 | 30.917 | 47.877 | 1.00 | 44.53 | C | |
| ATOM | 7437 | O | ILE | B | 432 | 58.600 | 30.193 | 47.437 | 1.00 | 44.41 | O | |
| ATOM | 7439 | N | ALA | B | 433 | 57.432 | 32.208 | 47.567 | 1.00 | 42.33 | N | |
| ATOM | 7440 | CA | ALA | B | 433 | 58.303 | 32.959 | 46.700 | 1.00 | 42.53 | C | |
| ATOM | 7442 | CB | ALA | B | 433 | 57.874 | 34.460 | 46.671 | 1.00 | 43.90 | C | |
| ATOM | 7446 | C | ALA | B | 433 | 59.797 | 32.889 | 47.022 | 1.00 | 42.39 | C | |
| ATOM | 7447 | O | ALA | B | 433 | 60.628 | 32.843 | 46.112 | 1.00 | 44.01 | O | |
| ATOM | 7449 | N | THR | B | 434 | 60.150 | 32.904 | 48.292 | 1.00 | 41.40 | N | |
| ATOM | 7450 | CA | THR | B | 434 | 61.538 | 32.810 | 48.692 | 1.00 | 40.51 | C | |
| ATOM | 7452 | CB | THR | B | 434 | 61.744 | 33.673 | 49.936 | 1.00 | 39.64 | C | |
| ATOM | 7454 | OG1 | THR | B | 434 | 60.861 | 33.275 | 50.996 | 1.00 | 38.03 | O | |
| ATOM | 7456 | CG2 | THR | B | 434 | 61.323 | 35.111 | 49.673 | 1.00 | 39.97 | C | |
| ATOM | 7460 | C | THR | B | 434 | 61.968 | 31.349 | 48.959 | 1.00 | 41.24 | C | |
| ATOM | 7461 | O | THR | B | 434 | 62.990 | 31.152 | 49.586 | 1.00 | 41.64 | O | |
| ATOM | 7463 | N | TYR | B | 435 | 61.209 | 30.320 | 48.547 | 1.00 | 41.60 | N | |
| ATOM | 7464 | CA | TYR | B | 435 | 61.531 | 28.902 | 48.933 | 1.00 | 41.87 | C | |
| ATOM | 7466 | CB | TYR | B | 435 | 62.670 | 28.341 | 48.072 | 1.00 | 40.58 | C | |
| ATOM | 7469 | CG | TYR | B | 435 | 62.278 | 28.202 | 46.638 | 1.00 | 39.86 | C | |
| ATOM | 7470 | CD1 | TYR | B | 435 | 62.362 | 29.303 | 45.747 | 1.00 | 38.77 | C | |
| ATOM | 7472 | CE1 | TYR | B | 435 | 61.971 | 29.189 | 44.422 | 1.00 | 37.75 | C | |
| ATOM | 7474 | CZ | TYR | B | 435 | 61.502 | 27.938 | 43.967 | 1.00 | 39.39 | C | |
| ATOM | 7475 | OH | TYR | B | 435 | 61.126 | 27.771 | 42.670 | 1.00 | 39.34 | O | |
| ATOM | 7477 | CE2 | TYR | B | 435 | 61.410 | 26.839 | 44.829 | 1.00 | 37.30 | C | |
| ATOM | 7479 | CD2 | TYR | B | 435 | 61.780 | 26.981 | 46.155 | 1.00 | 37.41 | C | |
| ATOM | 7481 | C | TYR | B | 435 | 61.842 | 28.732 | 50.438 | 1.00 | 43.41 | C | |
| ATOM | 7482 | O | TYR | B | 435 | 62.779 | 27.989 | 50.849 | 1.00 | 43.02 | O | |
| ATOM | 7484 | N | GLY | B | 436 | 61.067 | 29.472 | 51.239 | 1.00 | 44.71 | N | |
| ATOM | 7485 | CA | GLY | B | 436 | 61.017 | 29.303 | 52.684 | 1.00 | 46.27 | C | |
| ATOM | 7488 | C | GLY | B | 436 | 61.947 | 30.147 | 53.533 | 1.00 | 47.33 | C | |
| ATOM | 7489 | O | GLY | B | 436 | 62.348 | 29.748 | 54.636 | 1.00 | 48.29 | O | |
| ATOM | 7491 | N | MET | B | 437 | 62.285 | 31.331 | 53.071 | 1.00 | 48.59 | N | |
| ATOM | 7492 | CA | MET | B | 437 | 62.949 | 32.247 | 53.967 | 1.00 | 49.82 | C | |
| ATOM | 7494 | CB | MET | B | 437 | 63.387 | 33.490 | 53.264 | 1.00 | 49.54 | C | |
| ATOM | 7497 | CG | MET | B | 437 | 64.617 | 33.284 | 52.389 | 1.00 | 52.30 | C | |
| ATOM | 7500 | SD | MET | B | 437 | 65.234 | 34.919 | 51.796 | 1.00 | 55.51 | S | |
| ATOM | 7501 | CE | MET | B | 437 | 65.384 | 35.857 | 53.375 | 1.00 | 58.82 | C | |
| ATOM | 7505 | C | MET | B | 437 | 62.029 | 32.627 | 55.103 | 1.00 | 48.51 | C | |
| ATOM | 7506 | O | MET | B | 437 | 60.857 | 32.759 | 54.919 | 1.00 | 49.50 | O | |
| ATOM | 7508 | N | SER | B | 438 | 62.603 | 32.790 | 56.279 | 1.00 | 48.19 | N | |
| ATOM | 7509 | CA | SER | B | 438 | 61.949 | 33.390 | 57.432 | 1.00 | 47.57 | C | |
| ATOM | 7511 | CB | SER | B | 438 | 62.906 | 33.253 | 58.609 | 1.00 | 47.24 | C | |
| ATOM | 7514 | OG | SER | B | 438 | 62.259 | 33.689 | 59.777 | 1.00 | 52.44 | O | |
| ATOM | 7516 | C | SER | B | 438 | 61.598 | 34.907 | 57.174 | 1.00 | 46.76 | C | |
| ATOM | 7517 | O | SER | B | 438 | 62.484 | 35.664 | 56.763 | 1.00 | 46.75 | O | |
| ATOM | 7519 | N | PRO | B | 439 | 60.343 | 35.333 | 57.326 | 1.00 | 45.74 | N | |
| ATOM | 7520 | CA | PRO | B | 439 | 59.949 | 36.745 | 57.049 | 1.00 | 45.91 | C | |
| ATOM | 7522 | CB | PRO | B | 439 | 58.420 | 36.797 | 57.333 | 1.00 | 45.79 | C | |
| ATOM | 7525 | CG | PRO | B | 439 | 58.012 | 35.479 | 57.802 | 1.00 | 46.45 | C | |
| ATOM | 7528 | CD | PRO | B | 439 | 59.186 | 34.501 | 57.681 | 1.00 | 46.24 | C | |
| ATOM | 7531 | C | PRO | B | 439 | 60.682 | 37.859 | 57.831 | 1.00 | 45.00 | C | |
| ATOM | 7532 | O | PRO | B | 439 | 61.295 | 37.614 | 58.875 | 1.00 | 45.64 | O | |
| ATOM | 7533 | N | TYR | B | 440 | 60.634 | 39.056 | 57.274 | 1.00 | 43.58 | N | |
| ATOM | 7534 | CA | TYR | B | 440 | 61.341 | 40.215 | 57.787 | 1.00 | 43.59 | C | |
| ATOM | 7536 | CB | TYR | B | 440 | 60.533 | 40.869 | 58.897 | 1.00 | 43.85 | C | |
| ATOM | 7539 | CG | TYR | B | 440 | 59.109 | 41.156 | 58.513 | 1.00 | 45.19 | C | |
| ATOM | 7540 | CD1 | TYR | B | 440 | 58.745 | 42.332 | 57.850 | 1.00 | 48.24 | C | |
| ATOM | 7542 | CE1 | TYR | B | 440 | 57.416 | 42.572 | 57.496 | 1.00 | 46.93 | C | |
| ATOM | 7544 | CZ | TYR | B | 440 | 56.464 | 41.632 | 57.826 | 1.00 | 45.57 | C | |
| ATOM | 7545 | OH | TYR | B | 440 | 55.138 | 41.789 | 57.542 | 1.00 | 47.40 | O | |
| ATOM | 7547 | CE2 | TYR | B | 440 | 56.803 | 40.509 | 58.484 | 1.00 | 47.26 | C | |
| ATOM | 7549 | CD2 | TYR | B | 440 | 58.117 | 40.264 | 58.808 | 1.00 | 47.98 | C | |
| ATOM | 7551 | C | TYR | B | 440 | 62.753 | 39.898 | 58.249 | 1.00 | 42.40 | C | |
| ATOM | 7552 | O | TYR | B | 440 | 63.159 | 40.328 | 59.316 | 1.00 | 41.59 | O | |
| ATOM | 7554 | N | PRO | B | 441 | 63.507 | 39.155 | 57.439 | 1.00 | 41.60 | N | |
| ATOM | 7555 | CA | PRO | B | 441 | 64.761 | 38.512 | 57.925 | 1.00 | 41.97 | C | |
| ATOM | 7557 | CB | PRO | B | 441 | 65.323 | 37.833 | 56.678 | 1.00 | 41.25 | C | |
| ATOM | 7560 | CG | PRO | B | 441 | 64.672 | 38.557 | 55.539 | 1.00 | 41.33 | C | |
| ATOM | 7563 | CD | PRO | B | 441 | 63.302 | 38.941 | 55.997 | 1.00 | 41.06 | C | |
| ATOM | 7566 | C | PRO | B | 441 | 65.692 | 39.594 | 58.449 | 1.00 | 42.48 | C | |
| ATOM | 7567 | O | PRO | B | 441 | 65.757 | 40.689 | 57.825 | 1.00 | 44.88 | O | |
| ATOM | 7568 | N | GLY | B | 442 | 66.284 | 39.377 | 59.620 | 1.00 | 42.00 | N | |
| ATOM | 7569 | CA | GLY | B | 442 | 67.190 | 40.354 | 60.207 | 1.00 | 42.52 | C | |
| ATOM | 7572 | C | GLY | B | 442 | 66.545 | 41.421 | 61.066 | 1.00 | 42.98 | C | |
| ATOM | 7573 | O | GLY | B | 442 | 67.246 | 42.073 | 61.816 | 1.00 | 43.85 | O | |
| ATOM | 7575 | N | ILE | B | 443 | 65.223 | 41.583 | 60.991 | 1.00 | 43.59 | N | |
| ATOM | 7576 | CA | ILE | B | 443 | 64.467 | 42.596 | 61.729 | 1.00 | 43.65 | C | |
| ATOM | 7578 | CB | ILE | B | 443 | 63.274 | 43.076 | 60.912 | 1.00 | 43.19 | C | |
| ATOM | 7580 | CG1 | LE | B | 443 | 63.781 | 43.664 | 59.594 | 1.00 | 44.00 | C | |
| ATOM | 7583 | CD1 | ILE | B | 443 | 62.727 | 43.878 | 58.504 | 1.00 | 45.01 | C | |
| ATOM | 7587 | CG2 | ILE | B | 443 | 62.455 | 44.126 | 61.687 | 1.00 | 43.94 | C | |
| ATOM | 7591 | C | ILE | B | 443 | 63.977 | 41.980 | 63.042 | 1.00 | 44.13 | C | |
| ATOM | 7592 | O | ILE | B | 443 | 63.406 | 40.930 | 63.021 | 1.00 | 44.69 | O | |
| ATOM | 7594 | N | ASP | B | 444 | 64.176 | 42.678 | 64.165 | 1.00 | 45.09 | N | |
| ATOM | 7595 | CA | ASP | B | 444 | 63.785 | 42.217 | 65.479 | 1.00 | 45.27 | C | |
| ATOM | 7597 | CB | ASP | B | 444 | 64.455 | 43.059 | 66.580 | 1.00 | 46.01 | C | |
| ATOM | 7600 | CG | ASP | B | 444 | 64.394 | 42.383 | 67.976 | 1.00 | 50.38 | C | |
| ATOM | 7601 | OD1 | ASP | B | 444 | 65.232 | 41.480 | 68.279 | 1.00 | 54.48 | O | |
| ATOM | 7602 | OD2 | ASP | B | 444 | 63.532 | 42.693 | 68.846 | 1.00 | 56.55 | O | |
| ATOM | 7603 | C | ASP | B | 444 | 62.252 | 42.284 | 65.609 | 1.00 | 45.10 | C | |
| ATOM | 7604 | O | ASP | B | 444 | 61.603 | 43.248 | 65.149 | 1.00 | 45.64 | O | |
| ATOM | 7606 | N | LEU | B | 445 | 61.691 | 41.263 | 66.265 | 1.00 | 44.14 | N | |
| ATOM | 7607 | CA | LEU | B | 445 | 60.263 | 41.178 | 66.522 | 1.00 | 43.02 | C | |
| ATOM | 7609 | CB | LEU | B | 445 | 59.897 | 39.974 | 67.410 | 1.00 | 42.94 | C | |
| ATOM | 7612 | CG | LEU | B | 445 | 60.355 | 38.578 | 67.000 | 1.00 | 39.91 | C | |
| ATOM | 7614 | CD1 | LEU | B | 445 | 59.679 | 37.598 | 67.838 | 1.00 | 39.50 | C | |
| ATOM | 7618 | CD2 | LEU | B | 445 | 60.056 | 38.313 | 65.593 | 1.00 | 39.23 | C | |
| ATOM | 7622 | C | LEU | B | 445 | 59.739 | 42.397 | 67.185 | 1.00 | 42.88 | C | |
| ATOM | 7623 | O | LEU | B | 445 | 58.654 | 42.778 | 66.854 | 1.00 | 43.96 | O | |
| ATOM | 7625 | N | SER | B | 446 | 60.510 | 43.031 | 68.076 | 1.00 | 42.48 | N | |
| ATOM | 7626 | CA | SER | B | 446 | 60.078 | 44.261 | 68.708 | 1.00 | 43.16 | C | |
| ATOM | 7628 | CB | SER | B | 446 | 61.125 | 44.808 | 69.675 | 1.00 | 43.32 | C | |
| ATOM | 7631 | OG | SER | B | 446 | 62.318 | 45.279 | 69.026 | 1.00 | 46.55 | O | |
| ATOM | 7633 | C | SER | B | 446 | 59.696 | 45.358 | 67.734 | 1.00 | 43.33 | C | |
| ATOM | 7634 | O | SER | B | 446 | 58.947 | 46.232 | 68.121 | 1.00 | 43.82 | O | |
| ATOM | 7636 | N | GLN | B | 447 | 60.201 | 45.311 | 66.490 | 1.00 | 43.85 | N | |
| ATOM | 7637 | CA | GLN | B | 447 | 60.074 | 46.422 | 65.502 | 1.00 | 43.96 | C | |
| ATOM | 7639 | CB | GLN | B | 447 | 61.444 | 46.701 | 64.829 | 1.00 | 44.59 | C | |
| ATOM | 7642 | CG | GLN | B | 447 | 62.549 | 47.135 | 65.804 | 1.00 | 44.79 | C | |
| ATOM | 7645 | CD | GLN | B | 447 | 62.113 | 48.281 | 66.696 | 1.00 | 46.24 | C | |
| ATOM | 7646 | OE1 | GLN | B | 447 | 62.196 | 49.447 | 66.282 | 1.00 | 50.99 | O | |
| ATOM | 7647 | NE2 | GLN | B | 447 | 61.599 | 47.959 | 67.897 | 1.00 | 41.42 | N | |
| ATOM | 7650 | C | GLN | B | 447 | 59.020 | 46.256 | 64.446 | 1.00 | 43.16 | C | |
| ATOM | 7651 | O | GLN | B | 447 | 58.540 | 47.242 | 63.898 | 1.00 | 42.27 | O | |
| ATOM | 7653 | N | VAL | B | 448 | 58.601 | 45.015 | 64.256 | 1.00 | 43.37 | N | |
| ATOM | 7654 | CA | VAL | B | 448 | 57.756 | 44.597 | 63.149 | 1.00 | 43.31 | C | |
| ATOM | 7656 | CB | VAL | B | 448 | 57.659 | 43.063 | 63.125 | 1.00 | 43.23 | C | |
| ATOM | 7658 | CG1 | VAL | B | 448 | 56.556 | 42.551 | 62.179 | 1.00 | 45.60 | C | |
| ATOM | 7662 | CG2 | VAL | B | 448 | 59.015 | 42.492 | 62.716 | 1.00 | 42.14 | C | |
| ATOM | 7666 | C | VAL | B | 448 | 56.418 | 45.326 | 63.165 | 1.00 | 43.28 | C | |
| ATOM | 7667 | O | VAL | B | 448 | 56.092 | 45.929 | 62.166 | 1.00 | 44.31 | O | |
| ATOM | 7669 | N | TYR | B | 449 | 55.689 | 45.383 | 64.276 | 1.00 | 43.80 | N | |
| ATOM | 7670 | CA | TYR | B | 449 | 54.501 | 46.281 | 64.301 | 1.00 | 45.14 | C | |
| ATOM | 7672 | CB | TYR | B | 449 | 53.691 | 46.215 | 65.614 | 1.00 | 44.90 | C | |
| ATOM | 7675 | CG | TYR | B | 449 | 52.313 | 46.898 | 65.541 | 1.00 | 44.12 | C | |
| ATOM | 7676 | CD1 | TYR | B | 449 | 52.189 | 48.255 | 65.779 | 1.00 | 46.97 | C | |
| ATOM | 7678 | CE1 | TYR | B | 449 | 50.943 | 48.910 | 65.720 | 1.00 | 46.64 | C | |
| ATOM | 7680 | CZ | TYR | B | 449 | 49.815 | 48.190 | 65.447 | 1.00 | 45.85 | C | |
| ATOM | 7681 | OH | TYR | B | 449 | 48.665 | 48.911 | 65.395 | 1.00 | 46.60 | O | |
| ATOM | 7683 | CE2 | TYR | B | 449 | 49.885 | 46.829 | 65.221 | 1.00 | 44.32 | C | |
| ATOM | 7685 | CD2 | TYR | B | 449 | 51.136 | 46.187 | 65.266 | 1.00 | 44.41 | C | |
| ATOM | 7687 | C | TYR | B | 449 | 54.789 | 47.778 | 63.894 | 1.00 | 46.61 | C | |
| ATOM | 7688 | O | TYR | B | 449 | 54.067 | 48.298 | 63.000 | 1.00 | 47.56 | O | |
| ATOM | 7690 | N | GLU | B | 450 | 55.802 | 48.442 | 64.500 | 1.00 | 46.75 | N | |
| ATOM | 7691 | CA | GLU | B | 450 | 56.031 | 49.886 | 64.271 | 1.00 | 46.77 | C | |
| ATOM | 7693 | CB | GLU | B | 450 | 57.232 | 50.432 | 65.055 | 1.00 | 47.02 | C | |
| ATOM | 7696 | CG | GLU | B | 450 | 57.189 | 51.947 | 65.277 | 1.00 | 47.82 | C | |
| ATOM | 7699 | CD | GLU | B | 450 | 58.484 | 52.545 | 65.899 | 1.00 | 49.42 | C | |
| ATOM | 7700 | OE1 | GLU | B | 450 | 59.260 | 51.671 | 66.456 | 1.00 | 51.76 | O | |
| ATOM | 7701 | OE2 | GLU | B | 450 | 58.720 | 53.847 | 65.828 | 1.00 | 42.12 | O | |
| ATOM | 7702 | C | GLU | B | 450 | 56.266 | 50.117 | 62.807 | 1.00 | 46.93 | C | |
| ATOM | 7703 | O | GLU | B | 450 | 55.646 | 50.986 | 62.191 | 1.00 | 46.71 | O | |
| ATOM | 7705 | N | LEU | B | 451 | 57.138 | 49.276 | 62.251 | 1.00 | 46.89 | N | |
| ATOM | 7706 | CA | LEU | B | 451 | 57.477 | 49.294 | 60.816 | 1.00 | 46.73 | C | |
| ATOM | 7708 | CB | LEU | B | 451 | 58.482 | 48.152 | 60.492 | 1.00 | 46.28 | C | |
| ATOM | 7711 | CG | LEU | B | 451 | 59.903 | 48.347 | 61.060 | 1.00 | 44.24 | C | |
| ATOM | 7713 | CD1 | LEU | B 451 | 60.763 | 47.101 | 60.918 | 1.00 | 42.25 | C | | |
| ATOM | 7717 | CD2 | LEU | B | 451 | 60.600 | 49.581 | 60.468 | 1.00 | 42.74 | C | |
| ATOM | 7721 | C | LEU | B | 451 | 56.228 | 49.244 | 59.919 | 1.00 | 47.03 | C | |
| ATOM | 7722 | O | LEU | B | 451 | 56.016 | 50.176 | 59.149 | 1.00 | 47.32 | O | |
| ATOM | 7724 | N | LEU | B | 452 | 55.376 | 48.218 | 60.099 | 1.00 | 47.10 | N | |
| ATOM | 7725 | CA | LEU | B | 452 | 54.067 | 48.137 | 59.379 | 1.00 | 46.82 | C | |
| ATOM | 7727 | CB | LEU | B | 452 | 53.324 | 46.825 | 59.660 | 1.00 | 45.75 | C | |
| ATOM | 7730 | CG | LEU | B | 452 | 54.040 | 45.512 | 59.327 | 1.00 | 46.16 | C | |
| ATOM | 7732 | CD1 | LEU | B | 452 | 53.516 | 44.364 | 60.152 | 1.00 | 43.64 | C | |
| ATOM | 7736 | CD2 | LEU | B | 452 | 53.973 | 45.160 | 57.846 | 1.00 | 45.86 | C | |
| ATOM | 7740 | C | LEU | B | 452 | 53.134 | 49.344 | 59.645 | 1.00 | 46.45 | C | |
| ATOM | 7741 | O | LEU | B | 452 | 52.453 | 49.735 | 58.761 | 1.00 | 46.63 | O | |
| ATOM | 7743 | N | GLU | B | 453 | 53.111 | 49.942 | 60.832 | 1.00 | 47.02 | N | |
| ATOM | 7744 | CA | GLU | B | 453 | 52.306 | 51.169 | 61.015 | 1.00 | 47.90 | C | |
| ATOM | 7746 | CB | GLU | B | 453 | 52.112 | 51.543 | 62.504 | 1.00 | 48.34 | C | |
| ATOM | 7749 | CG | GLU | B | 453 | 51.057 | 52.632 | 62.779 | 1.00 | 49.22 | C | |
| ATOM | 7752 | CD | GLU | B | 453 | 50.411 | 52.592 | 64.195 | 1.00 | 52.09 | C | |
| ATOM | 7753 | OE1 | GLU | B | 453 | 51.162 | 52.344 | 65.194 | 1.00 | 58.06 | O | |
| ATOM | 7754 | OE2 | GLU | B | 453 | 49.143 | 52.831 | 64.318 | 1.00 | 55.89 | O | |
| ATOM | 7755 | C | GLU | B | 453 | 52.869 | 52.355 | 60.211 | 1.00 | 46.91 | C | |
| ATOM | 7756 | O | GLU | B | 453 | 52.130 | 53.220 | 59.837 | 1.00 | 46.20 | O | |
| ATOM | 7758 | N | LYS | B | 454 | 54.166 | 52.338 | 59.909 | 1.00 | 47.09 | N | |
| ATOM | 7759 | CA | LYS | B | 454 | 54.834 | 53.390 | 59.145 | 1.00 | 47.08 | C | |
| ATOM | 7761 | CB | LYS | B | 454 | 56.171 | 53.760 | 59.815 | 1.00 | 47.46 | C | |
| ATOM | 7764 | CG | LYS | B | 454 | 56.225 | 53.921 | 61.415 | 1.00 | 47.21 | C | |
| ATOM | 7767 | CD | LYS | B | 454 | 55.745 | 55.278 | 61.931 | 1.00 | 48.77 | C | |
| ATOM | 7770 | CE | LYS | B | 454 | 56.629 | 55.854 | 63.085 | 1.00 | 50.17 | C | |
| ATOM | 7773 | NZ | LYS | B | 454 | 56.923 | 54.873 | 64.195 | 1.00 | 51.07 | N | |
| ATOM | 7777 | C | LYS | B | 454 | 55.034 | 52.974 | 57.677 | 1.00 | 47.19 | C | |
| ATOM | 7778 | O | LYS | B | 454 | 55.969 | 53.404 | 57.020 | 1.00 | 48.03 | O | |
| ATOM | 7780 | N | ASP | B | 455 | 54.151 | 52.123 | 57.163 | 1.00 | 47.51 | N | |
| ATOM | 7781 | CA | ASP | B | 455 | 54.164 | 51.640 | 55.768 | 1.00 | 47.30 | C | |
| ATOM | 7783 | CB | ASP | B | 455 | 53.938 | 52.815 | 54.813 | 1.00 | 47.68 | C | |
| ATOM | 7786 | CG | ASP | B | 455 | 52.559 | 53.454 | 54.985 | 1.00 | 49.87 | C | |
| ATOM | 7787 | OD1 | ASP | B | 455 | 51.545 | 52.693 | 55.063 | 1.00 | 50.99 | O | |
| ATOM | 7788 | OD2 | ASP | B | 455 | 52.385 | 54.708 | 55.004 | 1.00 | 52.92 | O | |
| ATOM | 7789 | C | ASP | B | 455 | 55.312 | 50.716 | 55.278 | 1.00 | 47.15 | C | |
| ATOM | 7790 | O | ASP | B | 455 | 55.389 | 50.416 | 54.075 | 1.00 | 46.98 | O | |
| ATOM | 7792 | N | TYR | B | 456 | 56.155 | 50.209 | 56.183 | 1.00 | 46.38 | N | |
| ATOM | 7793 | CA | TYR | B | 456 | 57.193 | 49.272 | 55.763 | 1.00 | 46.01 | C | |
| ATOM | 7795 | CB | TYR | B | 456 | 58.177 | 48.922 | 56.880 | 1.00 | 45.30 | C | |
| ATOM | 7798 | CG | TYR | B | 456 | 59.257 | 47.970 | 56.385 | 1.00 | 43.90 | C | |
| ATOM | 7799 | CD1 | TYR | B | 456 | 60.371 | 48.432 | 55.661 | 1.00 | 43.05 | C | |
| ATOM | 7801 | CE1 | TYR | B | 456 | 61.309 | 47.535 | 55.153 | 1.00 | 44.00 | C | |
| ATOM | 7803 | CZ | TYR | B | 456 | 61.103 | 46.157 | 55.386 | 1.00 | 43.33 | C | |
| ATOM | 7804 | OH | TYR | B | 456 | 61.934 | 45.186 | 54.973 | 1.00 | 41.19 | O | |
| ATOM | 7806 | CE2 | TYR | B | 456 | 60.028 | 45.724 | 56.105 | 1.00 | 44.07 | C | |
| ATOM | 7808 | CD2 | TYR | B | 456 | 59.122 | 46.613 | 56.584 | 1.00 | 42.69 | C | |
| ATOM | 7810 | C | TYR | B | 456 | 56.532 | 47.988 | 55.299 | 1.00 | 46.18 | C | |
| ATOM | 7811 | O | TYR | B | 456 | 55.739 | 47.438 | 56.010 | 1.00 | 47.32 | O | |
| ATOM | 7813 | N | ARG | B | 457 | 56.863 | 47.516 | 54.115 | 1.00 | 45.87 | N | |
| ATOM | 7814 | CA | ARG | B | 457 | 56.539 | 46.162 | 53.718 | 1.00 | 45.60 | C | |
| ATOM | 7816 | CB | ARG | B | 457 | 55.344 | 46.125 | 52.767 | 1.00 | 45.00 | C | |
| ATOM | 7819 | CG | ARG | B | 457 | 54.072 | 46.825 | 53.212 | 1.00 | 44.26 | C | |
| ATOM | 7822 | CD | ARG | B | 457 | 53.132 | 45.939 | 54.022 | 1.00 | 44.61 | C | |
| ATOM | 7825 | NE | ARG | B | 457 | 51.867 | 46.571 | 54.412 | 1.00 | 43.28 | N | |
| ATOM | 7827 | CZ | ARG | B | 457 | 51.750 | 47.491 | 55.356 | 1.00 | 43.74 | C | |
| ATOM | 7828 | NH1 | ARG | B | 457 | 52.821 | 47.969 | 55.988 | 1.00 | 44.12 | N | |
| ATOM | 7831 | NH2 | ARG | B | 457 | 50.551 | 47.985 | 55.648 | 1.00 | 43.36 | N | |
| ATOM | 7834 | C | ARG | B | 457 | 57.767 | 45.558 | 53.025 | 1.00 | 46.59 | C | |
| ATOM | 7835 | O | ARG | B | 457 | 58.593 | 46.286 | 52.414 | 1.00 | 46.60 | O | |
| ATOM | 7837 | N | MET | B | 458 | 57.823 | 44.223 | 53.098 | 1.00 | 47.07 | N | |
| ATOM | 7838 | CA | MET | B | 458 | 58.823 | 43.403 | 52.448 | 1.00 | 47.52 | C | |
| ATOM | 7840 | CB | MET | B | 458 | 58.515 | 41.912 | 52.654 | 1.00 | 46.61 | C | |
| ATOM | 7843 | CG | MET | B | 458 | 58.680 | 41.536 | 54.097 | 1.00 | 46.82 | C | |
| ATOM | 7846 | SD | MET | B | 458 | 58.772 | 39.806 | 54.462 | 1.00 | 49.50 | S | |
| ATOM | 7847 | CE | MET | B | 458 | 57.034 | 39.367 | 54.436 | 1.00 | 52.19 | C | |
| ATOM | 7851 | C | MET | B | 458 | 58.901 | 43.704 | 50.969 | 1.00 | 48.40 | C | |
| ATOM | 7852 | O | MET | B | 458 | 57.869 | 43.829 | 50.313 | 1.00 | 47.93 | O | |
| ATOM | 7854 | N | GLU | B | 459 | 60.136 | 43.863 | 50.482 | 1.00 | 49.94 | N | |
| ATOM | 7855 | CA | GLU | B | 459 | 60.465 | 43.939 | 49.059 | 1.00 | 51.52 | C | |
| ATOM | 7857 | CB | GLU | B | 459 | 61.978 | 44.017 | 48.864 | 1.00 | 51.77 | C | |
| ATOM | 7860 | CG | GLU | B | 459 | 62.653 | 45.350 | 49.118 | 1.00 | 53.89 | C | |
| ATOM | 7863 | CD | GLU | B | 459 | 64.184 | 45.195 | 49.212 | 1.00 | 56.32 | C | |
| ATOM | 7864 | OE1 | GLU | B | 459 | 64.651 | 44.347 | 50.059 | 1.00 | 61.08 | O | |
| ATOM | 7865 | OE2 | GLU | B | 459 | 64.922 | 45.939 | 48.476 | 1.00 | 61.82 | O | |
| ATOM | 7866 | C | GLU | B | 459 | 60.040 | 42.681 | 48.308 | 1.00 | 51.00 | C | |
| ATOM | 7867 | O | GLU | B | 459 | 59.965 | 41.589 | 48.878 | 1.00 | 50.53 | O | |
| ATOM | 7869 | N | ARG | B | 460 | 59.824 | 42.852 | 47.008 | 1.00 | 51.42 | N | |
| ATOM | 7870 | CA | ARG | B | 460 | 59.517 | 41.747 | 46.112 | 1.00 | 52.12 | C | |
| ATOM | 7872 | CB | ARG | B | 460 | 59.191 | 42.238 | 44.703 | 1.00 | 51.56 | C | |
| ATOM | 7875 | CG | ARG | B | 460 | 58.867 | 41.133 | 43.748 | 1.00 | 53.27 | C | |
| ATOM | 7878 | CD | ARG | B | 460 | 58.227 | 41.557 | 42.401 | 1.00 | 55.17 | C | |
| ATOM | 7881 | NE | ARG | B | 460 | 59.137 | 42.265 | 41.499 | 1.00 | 58.14 | N | |
| ATOM | 7883 | CZ | ARG | B | 460 | 60.200 | 41.729 | 40.875 | 1.00 | 60.71 | C | |
| ATOM | 7884 | NH1 | ARG | B | 460 | 60.555 | 40.436 | 41.024 | 1.00 | 63.60 | N | |
| ATOM | 7887 | NH2 | ARG | B | 460 | 60.939 | 42.502 | 40.084 | 1.00 | 59.53 | N | |
| ATOM | 7890 | C | ARG | B | 460 | 60.735 | 40.817 | 46.110 | 1.00 | 51.66 | C | |
| ATOM | 7891 | O | ARG | B | 460 | 61.842 | 41.308 | 45.908 | 1.00 | 52.31 | O | |
| ATOM | 7893 | N | PRO | B | 461 | 60.564 | 39.527 | 46.404 | 1.00 | 50.97 | N | |
| ATOM | 7894 | CA | PRO | B | 461 | 61.670 | 38.586 | 46.289 | 1.00 | 51.56 | C | |
| ATOM | 7896 | CB | PRO | B | 461 | 61.064 | 37.273 | 46.789 | 1.00 | 51.60 | C | |
| ATOM | 7899 | CG | PRO | B | 461 | 59.939 | 37.687 | 47.600 | 1.00 | 51.63 | C | |
| ATOM | 7902 | CD | PRO | B | 461 | 59.360 | 38.881 | 46.949 | 1.00 | 50.26 | C | |
| ATOM | 7905 | C | PRO | B | 461 | 62.135 | 38.391 | 44.870 | 1.00 | 52.44 | C | |
| ATOM | 7906 | O | PRO | B | 461 | 61.319 | 38.473 | 43.965 | 1.00 | 53.32 | O | |
| ATOM | 7907 | N | GLU | B | 462 | 63.410 | 38.080 | 44.675 | 1.00 | 53.32 | N | |
| ATOM | 7908 | CA | GLU | B | 462 | 63.896 | 37.742 | 43.347 | 1.00 | 55.00 | C | |
| ATOM | 7910 | CB | GLU | B | 462 | 65.366 | 37.234 | 43.365 | 1.00 | 56.22 | C | |
| ATOM | 7913 | CG | GLU | B | 462 | 66.410 | 38.183 | 43.949 | 1.00 | 60.07 | C | |
| ATOM | 7916 | CD | GLU | B | 462 | 66.221 | 39.622 | 43.490 | 1.00 | 65.95 | C | |
| ATOM | 7917 | OE1 | GLU | B | 462 | 66.238 | 39.824 | 42.249 | 1.00 | 71.53 | O | |
| ATOM | 7918 | OE2 | GLU | B | 462 | 66.020 | 40.538 | 44.347 | 1.00 | 68.75 | O | |
| ATOM | 7919 | C | GLU | B | 462 | 63.029 | 36.654 | 42.741 | 1.00 | 54.43 | C | |
| ATOM | 7920 | O | GLU | B | 462 | 62.689 | 35.715 | 43.414 | 1.00 | 54.88 | O | |
| ATOM | 7922 | N | GLY | B | 463 | 62.644 | 36.816 | 41.490 | 1.00 | 54.46 | N | |
| ATOM | 7923 | CA | GLY | B | 463 | 61.936 | 35.782 | 40.759 | 1.00 | 54.77 | C | |
| ATOM | 7926 | C | GLY | B | 463 | 60.438 | 35.916 | 40.849 | 1.00 | 55.50 | C | |
| ATOM | 7927 | O | GLY | B | 463 | 59.733 | 35.283 | 40.076 | 1.00 | 56.51 | O | |
| ATOM | 7929 | N | CYS | B | 464 | 59.937 | 36.745 | 41.764 | 1.00 | 55.65 | N | |
| ATOM | 7930 | CA | CYS | B | 464 | 58.516 | 36.818 | 42.024 | 1.00 | 55.75 | C | |
| ATOM | 7932 | CB | CYS | B | 464 | 58.234 | 37.571 | 43.317 | 1.00 | 55.49 | C | |
| ATOM | 7935 | SG | CYS | B | 464 | 56.483 | 37.585 | 43.734 | 1.00 | 55.19 | S | |
| ATOM | 7937 | C | CYS | B | 464 | 57.867 | 37.558 | 40.872 | 1.00 | 55.99 | C | |
| ATOM | 7938 | O | CYS | B | 464 | 58.243 | 38.693 | 40.633 | 1.00 | 57.00 | O | |
| ATOM | 7940 | N | PRO | B | 465 | 56.906 | 36.954 | 40.155 | 1.00 | 55.91 | N | |
| ATOM | 7941 | CA | PRO | B | 465 | 56.206 | 37.657 | 39.086 | 1.00 | 55.39 | C | |
| ATOM | 7943 | CB | PRO | B | 465 | 55.098 | 36.677 | 38.734 | 1.00 | 55.24 | C | |
| ATOM | 7946 | CG | PRO | B | 465 | 55.657 | 35.352 | 39.061 | 1.00 | 54.51 | C | |
| ATOM | 7949 | CD | PRO | B | 465 | 56.393 | 35.571 | 40.294 | 1.00 | 55.51 | C | |
| ATOM | 7952 | C | PRO | B | 465 | 55.609 | 38.942 | 39.589 | 1.00 | 55.73 | C | |
| ATOM | 7953 | O | PRO | B | 465 | 55.166 | 38.954 | 40.707 | 1.00 | 56.93 | O | |
| ATOM | 7954 | N | GLU | B | 466 | 55.606 | 40.009 | 38.807 | 1.00 | 56.49 | N | |
| ATOM | 7955 | CA | GLU | B | 466 | 54.979 | 41.266 | 39.245 | 1.00 | 57.21 | C | |
| ATOM | 7957 | CB | GLU | B | 466 | 55.014 | 42.369 | 38.153 | 1.00 | 57.15 | C | |
| ATOM | 7960 | CG | GLU | B | 466 | 54.784 | 43.809 | 38.667 | 1.00 | 57.94 | C | |
| ATOM | 7963 | CD | GLU | B | 466 | 54.686 | 44.919 | 37.545 | 1.00 | 60.39 | C | |
| ATOM | 7964 | OE1 | GLU | B | 466 | 55.141 | 44.722 | 36.373 | 1.00 | 63.82 | O | |
| ATOM | 7965 | OE2 | GLU | B | 466 | 54.140 | 46.036 | 37.836 | 1.00 | 62.32 | O | |
| ATOM | 7966 | C | GLU | B | 466 | 53.533 | 41.095 | 39.782 | 1.00 | 56.91 | C | |
| ATOM | 7967 | O | GLU | B | 466 | 53.201 | 41.717 | 40.805 | 1.00 | 57.75 | O | |
| ATOM | 7969 | N | LYS | B | 467 | 52.695 | 40.279 | 39.128 | 1.00 | 55.96 | N | |
| ATOM | 7970 | CA | LYS | B | 467 | 51.253 | 40.223 | 39.480 | 1.00 | 55.82 | C | |
| ATOM | 7972 | CB | LYS | B | 467 | 50.407 | 39.508 | 38.409 | 1.00 | 56.45 | C | |
| ATOM | 7975 | CG | LYS | B | 467 | 50.496 | 40.106 | 36.968 | 1.00 | 58.90 | C | |
| ATOM | 7978 | CD | LYS | B | 467 | 49.677 | 41.455 | 36.725 | 1.00 | 61.23 | C | |
| ATOM | 7981 | CE | LYS | B | 467 | 50.190 | 42.236 | 35.437 | 1.00 | 60.98 | C | |
| ATOM | 7984 | NZ | LYS | B | 467 | 49.391 | 43.432 | 34.959 | 1.00 | 60.57 | N | |
| ATOM | 7988 | C | LYS | B | 467 | 51.042 | 39.580 | 40.865 | 1.00 | 54.38 | C | |
| ATOM | 7989 | O | LYS | B | 467 | 50.220 | 40.057 | 41.665 | 1.00 | 54.26 | O | |
| ATOM | 7991 | N | VAL | B | 468 | 51.817 | 38.532 | 41.151 | 1.00 | 52.45 | N | |
| ATOM | 7992 | CA | VAL | B | 468 | 51.845 | 37.905 | 42.480 | 1.00 | 50.89 | C | |
| ATOM | 7994 | CB | VAL | B | 468 | 52.756 | 36.667 | 42.556 | 1.00 | 49.97 | C | |
| ATOM | 7996 | CG1 | VAL | B | 468 | 52.663 | 36.048 | 43.927 | 1.00 | 49.26 | C | |
| ATOM | 8000 | CG2 | VAL | B | 468 | 52.377 | 35.630 | 41.523 | 1.00 | 49.97 | C | |
| ATOM | 8004 | C | VAL | B | 468 | 52.327 | 38.902 | 43.543 | 1.00 | 50.42 | C | |
| ATOM | 8005 | O | VAL | B | 468 | 51.791 | 38.933 | 44.641 | 1.00 | 50.86 | O | |
| ATOM | 8007 | N | TYR | B | 469 | 53.341 | 39.702 | 43.241 | 1.00 | 49.56 | N | |
| ATOM | 8008 | CA | TYR | B | 469 | 53.744 | 40.711 | 44.203 | 1.00 | 49.49 | C | |
| ATOM | 8010 | CB | TYR | B | 469 | 55.086 | 41.340 | 43.854 | 1.00 | 48.31 | C | |
| ATOM | 8013 | CG | TYR | B | 469 | 55.537 | 42.273 | 44.950 | 1.00 | 46.63 | C | |
| ATOM | 8014 | CD1 | TYR | B | 469 | 56.017 | 41.773 | 46.159 | 1.00 | 44.76 | C | |
| ATOM | 8016 | CE1 | TYR | B | 469 | 56.417 | 42.620 | 47.191 | 1.00 | 45.47 | C | |
| ATOM | 8018 | CZ | TYR | B | 469 | 56.318 | 43.999 | 47.018 | 1.00 | 45.67 | C | |
| ATOM | 8019 | OH | TYR | B | 469 | 56.707 | 44.844 | 48.020 | 1.00 | 45.88 | O | |
| ATOM | 8021 | CE2 | TYR | B | 469 | 55.823 | 44.521 | 45.831 | 1.00 | 44.67 | C | |
| ATOM | 8023 | CD2 | TYR | B | 469 | 55.423 | 43.651 | 44.805 | 1.00 | 44.44 | C | |
| ATOM | 8025 | C | TYR | B | 469 | 52.650 | 41.777 | 44.455 | 1.00 | 49.56 | C | |
| ATOM | 8026 | O | TYR | B | 469 | 52.408 | 42.098 | 45.605 | 1.00 | 49.84 | O | |
| ATOM | 8028 | N | GLU | B | 470 | 51.975 | 42.280 | 43.412 | 1.00 | 50.08 | N | |
| ATOM | 8029 | CA | GLU | B | 470 | 50.776 | 43.167 | 43.604 | 1.00 | 51.03 | C | |
| ATOM | 8031 | CB | GLU | B | 470 | 50.042 | 43.479 | 42.294 | 1.00 | 51.25 | C | |
| ATOM | 8034 | CG | GLU | B | 470 | 50.761 | 44.178 | 41.155 | 1.00 | 52.80 | C | |
| ATOM | 8037 | CD | GLU | B | 470 | 49.904 | 44.177 | 39.865 | 1.00 | 54.27 | C | |
| ATOM | 8038 | OE1 | GLU | B | 470 | 48.620 | 44.263 | 39.963 | 1.00 | 57.20 | O | |
| ATOM | 8039 | OE2 | GLU | B | 470 | 50.512 | 44.065 | 38.748 | 1.00 | 57.98 | O | |
| ATOM | 8040 | C | GLU | B | 470 | 49.693 | 42.555 | 44.530 | 1.00 | 50.19 | C | |
| ATOM | 8041 | O | GLU | B | 470 | 48.992 | 43.248 | 45.228 | 1.00 | 49.47 | O | |
| ATOM | 8043 | N | LEU | B | 471 | 49.555 | 41.246 | 44.468 | 1.00 | 50.05 | N | |
| ATOM | 8044 | CA | LEU | B | 471 | 48.568 | 40.516 | 45.245 | 1.00 | 50.26 | C | |
| ATOM | 8046 | CB | LEU | B | 471 | 48.442 | 39.125 | 44.632 | 1.00 | 50.49 | C | |
| ATOM | 8049 | CG | LEU | B | 471 | 47.186 | 38.353 | 44.948 | 1.00 | 52.40 | C | |
| ATOM | 8051 | CD1 | LEU | B | 471 | 46.063 | 38.682 | 44.017 | 1.00 | 53.17 | C | |
| ATOM | 8055 | CD2 | LEU | B | 471 | 47.481 | 36.903 | 44.856 | 1.00 | 54.74 | C | |
| ATOM | 8059 | C | LEU | B | 471 | 48.995 | 40.438 | 46.719 | 1.00 | 49.96 | C | |
| ATOM | 8060 | O | LEU | B | 471 | 48.180 | 40.571 | 47.639 | 1.00 | 49.37 | O | |
| ATOM | 8062 | N | MET | B | 472 | 50.288 | 40.192 | 46.932 | 1.00 | 50.03 | N | |
| ATOM | 8063 | CA | MET | B | 472 | 50.899 | 40.318 | 48.270 | 1.00 | 49.75 | C | |
| ATOM | 8065 | CB | MET | B | 472 | 52.400 | 40.062 | 48.205 | 1.00 | 49.58 | C | |
| ATOM | 8068 | CG | MET | B | 472 | 52.765 | 38.643 | 48.319 | 1.00 | 50.04 | C | |
| ATOM | 8071 | SD | MET | B | 472 | 54.588 | 38.660 | 48.414 | 1.00 | 51.23 | S | |
| ATOM | 8072 | CE | MET | B | 472 | 54.948 | 37.798 | 46.897 | 1.00 | 50.71 | C | |
| ATOM | 8076 | C | MET | B | 472 | 50.643 | 41.681 | 48.954 | 1.00 | 48.16 | C | |
| ATOM | 8077 | O | MET | B | 472 | 50.235 | 41.719 | 50.119 | 1.00 | 48.22 | O | |
| ATOM | 8079 | N | ARG | B | 473 | 50.845 | 42.765 | 48.231 | 1.00 | 46.63 | N | |
| ATOM | 8080 | CA | ARG | B | 473 | 50.634 | 44.086 | 48.813 | 1.00 | 47.12 | C | |
| ATOM | 8082 | CB | ARG | B | 473 | 51.240 | 45.172 | 47.934 | 1.00 | 46.99 | C | |
| ATOM | 8085 | CG | ARG | B | 473 | 52.727 | 45.018 | 47.741 | 1.00 | 46.95 | C | |
| ATOM | 8088 | CD | ARG | B | 473 | 53.568 | 45.322 | 48.972 | 1.00 | 48.50 | C | |
| ATOM | 8091 | NE | ARG | B | 473 | 54.309 | 46.574 | 48.824 | 1.00 | 49.47 | N | |
| ATOM | 8093 | CZ | ARG | B | 473 | 53.871 | 47.795 | 49.135 | 1.00 | 51.77 | C | |
| ATOM | 8094 | NH1 | ARG | B | 473 | 52.659 | 48.006 | 49.626 | 1.00 | 57.09 | N | |
| ATOM | 8097 | NH2 | ARG | B | 473 | 54.670 | 48.841 | 48.960 | 1.00 | 51.95 | N | |
| ATOM | 8100 | C | ARG | B | 473 | 49.143 | 44.358 | 49.091 | 1.00 | 47.30 | C | |
| ATOM | 8101 | O | ARG | B | 473 | 48.779 | 44.882 | 50.154 | 1.00 | 48.08 | O | |
| ATOM | 8103 | N | ALA | B | 474 | 48.296 | 43.963 | 48.148 | 1.00 | 47.28 | N | |
| ATOM | 8104 | CA | ALA | B | 474 | 46.858 | 43.844 | 48.357 | 1.00 | 47.10 | C | |
| ATOM | 8106 | CB | ALA | B | 474 | 46.203 | 43.046 | 47.217 | 1.00 | 46.89 | C | |
| ATOM | 8110 | C | ALA | B | 474 | 46.547 | 43.180 | 49.676 | 1.00 | 47.08 | C | |
| ATOM | 8111 | O | ALA | B | 474 | 45.931 | 43.801 | 50.502 | 1.00 | 48.05 | O | |
| ATOM | 8113 | N | CYS | B | 475 | 47.006 | 41.943 | 49.877 | 1.00 | 46.53 | N | |
| ATOM | 8114 | CA | CYS | B | 475 | 46.862 | 41.252 | 51.155 | 1.00 | 46.29 | C | |
| ATOM | 8116 | CB | CYS | B | 475 | 47.602 | 39.930 | 51.108 | 1.00 | 47.03 | C | |
| ATOM | 8119 | SG | CYS | B | 475 | 46.791 | 38.773 | 50.037 | 1.00 | 46.81 | S | |
| ATOM | 8121 | C | CYS B | 475 | 47.370 | 41.986 | 52.394 | 1.00 | 45.96 | C | | |
| ATOM | 8122 | O | CYS | B | 475 | 46.812 | 41.803 | 53.470 | 1.00 | 46.32 | O | |
| ATOM | 8124 | N | TRP | B | 476 | 48.435 | 42.774 | 52.253 | 1.00 | 45.16 | N | |
| ATOM | 8125 | CA | TRP | B | 476 | 49.005 | 43.505 | 53.373 | 1.00 | 44.35 | C | |
| ATOM | 8127 | CB | TRP | B | 476 | 50.538 | 43.489 | 53.294 | 1.00 | 43.69 | C | |
| ATOM | 8130 | CG | TRP | B | 476 | 51.166 | 42.135 | 53.267 | 1.00 | 42.95 | C | |
| ATOM | 8131 | CD1 | TRP | B | 476 | 50.685 | 40.958 | 53.826 | 1.00 | 42.95 | C | |
| ATOM | 8133 | NE1 | TRP | B | 476 | 51.552 | 39.915 | 53.589 | 1.00 | 41.25 | N | |
| ATOM | 8135 | CE2 | TRP | B | 476 | 52.626 | 40.400 | 52.890 | 1.00 | 42.10 | C | |
| ATOM | 8136 | CD2 | TRP | B | 476 | 52.413 | 41.810 | 52.688 | 1.00 | 41.95 | C | |
| ATOM | 8137 | CE3 | TRP | B | 476 | 53.383 | 42.551 | 51.997 | 1.00 | 40.35 | C | |
| ATOM | 8139 | CZ3 | TRP | B | 476 | 54.509 | 41.906 | 51.542 | 1.00 | 41.78 | C | |
| ATOM | 8141 | CH2 | TRP | B | 476 | 54.705 | 40.507 | 51.761 | 1.00 | 42.29 | C | |
| ATOM | 8143 | CZ2 | TRP | B | 476 | 53.774 | 39.745 | 52.431 | 1.00 | 42.28 | C | |
| ATOM | 8145 | C | TRP | B | 476 | 48.475 | 44.933 | 53.528 | 1.00 | 44.73 | C | |
| ATOM | 8146 | O | TRP | B | 476 | 49.132 | 45.766 | 54.161 | 1.00 | 45.30 | O | |
| ATOM | 8148 | N | GLN | B | 477 | 47.279 | 45.232 | 53.011 | 1.00 | 45.10 | N | |
| ATOM | 8149 | CA | GLN | B | 477 | 46.660 | 46.557 | 53.191 | 1.00 | 44.33 | C | |
| ATOM | 8151 | CB | GLN | B | 477 | 45.328 | 46.697 | 52.439 | 1.00 | 44.15 | C | |
| ATOM | 8154 | CG | GLN | B | 477 | 45.390 | 46.869 | 50.951 | 1.00 | 44.14 | C | |
| ATOM | 8157 | CD | GLN | B | 477 | 46.267 | 47.992 | 50.544 | 1.00 | 45.70 | C | |
| ATOM | 8158 | OE1 | GLN | B | 477 | 45.975 | 49.174 | 50.840 | 1.00 | 48.54 | O | |
| ATOM | 8159 | NE2 | GLN | B | 477 | 47.374 | 47.654 | 49.883 | 1.00 | 45.18 | N | |
| ATOM | 8162 | C | GLN | B | 477 | 46.369 | 46.770 | 54.666 | 1.00 | 44.57 | C | |
| ATOM | 8163 | O | GLN | B | 477 | 45.794 | 45.920 | 55.300 | 1.00 | 42.91 | O | |
| ATOM | 8165 | N | TRP | B | 478 | 46.739 | 47.949 | 55.170 | 1.00 | 45.68 | N | |
| ATOM | 8166 | CA | TRP | B | 478 | 46.503 | 48.331 | 56.549 | 1.00 | 46.05 | C | |
| ATOM | 8168 | CB | TRP | B | 478 | 46.877 | 49.800 | 56.805 | 1.00 | 44.79 | C | |
| ATOM | 8171 | CG | TRP | B | 478 | 46.815 | 50.101 | 58.282 | 1.00 | 44.68 | C | |
| ATOM | 8172 | CD1 | TRP | B | 478 | 45.826 | 50.765 | 58.950 | 1.00 | 43.03 | C | |
| ATOM | 8174 | NE1 | TRP | B | 478 | 46.086 | 50.754 | 60.301 | 1.00 | 42.19 | N | |
| ATOM | 8176 | CE2 | TRP | B | 478 | 47.267 | 50.096 | 60.533 | 1.00 | 42.05 | C | |
| ATOM | 8177 | CD2 | TRP | B | 478 | 47.743 | 49.642 | 59.288 | 1.00 | 43.64 | C | |
| ATOM | 8178 | CE3 | TRP | B | 478 | 48.950 | 48.912 | 59.253 | 1.00 | 42.44 | C | |
| ATOM | 8180 | CZ3 | TRP | B | 478 | 49.624 | 48.656 | 60.432 | 1.00 | 41.99 | C | |
| ATOM | 8182 | CH2 | TRP | B | 478 | 49.119 | 49.106 | 61.652 | 1.00 | 43.86 | C | |
| ATOM | 8184 | CZ2 | TRP | B | 478 | 47.951 | 49.851 | 61.724 | 1.00 | 43.39 | C | |
| ATOM | 8186 | C | TRP | B | 478 | 45.066 | 48.075 | 56.984 | 1.00 | 46.83 | C | |
| ATOM | 8187 | O | TRP | B | 478 | 44.876 | 47.405 | 58.001 | 1.00 | 48.39 | O | |
| ATOM | 8189 | N | ASN | B | 479 | 44.081 | 48.548 | 56.210 | 1.00 | 47.58 | N | |
| ATOM | 8190 | CA | ASN | B | 479 | 42.655 | 48.343 | 56.531 | 1.00 | 48.38 | C | |
| ATOM | 8192 | CB | ASN | B | 479 | 41.773 | 49.455 | 55.969 | 1.00 | 49.73 | C | |
| ATOM | 8195 | CG | ASN | B | 479 | 40.468 | 49.605 | 56.752 | 1.00 | 54.19 | C | |
| ATOM | 8196 | OD1 | ASN | B | 479 | 40.455 | 50.208 | 57.827 | 1.00 | 61.13 | O | |
| ATOM | 8197 | ND2 | ASN | B | 479 | 39.377 | 49.021 | 56.241 | 1.00 | 57.99 | N | |
| ATOM | 8200 | C | ASN | B | 479 | 42.104 | 46.992 | 56.012 | 1.00 | 48.60 | C | |
| ATOM | 8201 | O | ASN | B | 479 | 42.282 | 46.670 | 54.822 | 1.00 | 49.02 | O | |
| ATOM | 8203 | N | PRO | B | 480 | 41.410 | 46.222 | 56.850 | 1.00 | 47.90 | N | |
| ATOM | 8204 | CA | PRO | B | 480 | 40.840 | 44.936 | 56.406 | 1.00 | 47.89 | C | |
| ATOM | 8206 | CB | PRO | B | 480 | 40.102 | 44.449 | 57.643 | 1.00 | 47.90 | C | |
| ATOM | 8209 | CG | PRO | B | 480 | 40.791 | 45.134 | 58.780 | 1.00 | 47.61 | C | |
| ATOM | 8212 | CD | PRO | B | 480 | 41.119 | 46.488 | 58.270 | 1.00 | 47.35 | C | |
| ATOM | 8215 | C | PRO | B | 480 | 39.873 | 45.005 | 55.208 | 1.00 | 48.18 | C | |
| ATOM | 8216 | O | PRO | B | 480 | 39.925 | 44.141 | 54.331 | 1.00 | 48.11 | O | |
| ATOM | 8217 | N | SER | B | 481 | 39.018 | 46.032 | 55.158 | 1.00 | 48.48 | N | |
| ATOM | 8218 | CA | SER | B | 481 | 38.078 | 46.196 | 54.032 | 1.00 | 48.57 | C | |
| ATOM | 8220 | CB | SER | B | 481 | 37.100 | 47.322 | 54.277 | 1.00 | 48.17 | C | |
| ATOM | 8223 | OG | SER | B | 481 | 37.739 | 48.562 | 54.140 | 1.00 | 49.08 | O | |
| ATOM | 8225 | C | SER | B | 481 | 38.771 | 46.377 | 52.677 | 1.00 | 49.17 | C | |
| ATOM | 8226 | O | SER | B | 481 | 38.201 | 45.959 | 51.659 | 1.00 | 49.48 | O | |
| ATOM | 8228 | N | ASP | B | 482 | 40.004 | 46.938 | 52.679 | 1.00 | 49.58 | N | |
| ATOM | 8229 | CA | ASP | B | 482 | 40.827 | 47.113 | 51.447 | 1.00 | 49.26 | C | |
| ATOM | 8231 | CB | ASP | B | 482 | 42.024 | 48.071 | 51.609 | 1.00 | 49.40 | C | |
| ATOM | 8234 | CG | ASP | B | 482 | 41.651 | 49.465 | 52.133 | 1.00 | 50.76 | C | |
| ATOM | 8235 | OD1 | ASP | B | 482 | 40.531 | 49.993 | 51.875 | 1.00 | 51.54 | O | |
| ATOM | 8236 | OD2 | ASP | B | 482 | 42.497 | 50.105 | 52.813 | 1.00 | 51.94 | O | |
| ATOM | 8237 | C | ASP | B | 482 | 41.410 | 45.834 | 50.882 | 1.00 | 48.65 | C | |
| ATOM | 8238 | O | ASP | B | 482 | 41.828 | 45.838 | 49.734 | 1.00 | 48.82 | O | |
| ATOM | 8240 | N | ARG | B | 483 | 41.482 | 44.761 | 51.655 | 1.00 | 48.25 | N | |
| ATOM | 8241 | CA | ARG | B | 483 | 42.185 | 43.571 | 51.187 | 1.00 | 48.07 | C | |
| ATOM | 8243 | CB | ARG | B | 483 | 42.632 | 42.728 | 52.350 | 1.00 | 48.03 | C | |
| ATOM | 8246 | CG | ARG | B | 483 | 43.693 | 43.359 | 53.210 | 1.00 | 48.47 | C | |
| ATOM | 8249 | CD | ARG | B | 483 | 43.895 | 42.633 | 54.535 | 1.00 | 48.62 | C | |
| ATOM | 8252 | NE | ARG | B | 483 | 44.537 | 43.512 | 55.501 | 1.00 | 49.16 | N | |
| ATOM | 8254 | CZ | ARG | B | 483 | 44.435 | 43.400 | 56.824 | 1.00 | 51.38 | C | |
| ATOM | 8255 | NH1 | ARG | B | 483 | 43.804 | 42.358 | 57.380 | 1.00 | 52.50 | N | |
| ATOM | 8258 | NH2 | ARG | B | 483 | 45.004 | 44.314 | 57.618 | 1.00 | 49.87 | N | |
| ATOM | 8261 | C | ARG | B | 483 | 41.197 | 42.774 | 50.385 | 1.00 | 48.15 | C | |
| ATOM | 8262 | O | ARG | B | 483 | 40.048 | 42.787 | 50.764 | 1.00 | 48.56 | O | |
| ATOM | 8264 | N | PRO | B | 484 | 41.609 | 42.029 | 49.350 | 1.00 | 48.11 | N | |
| ATOM | 8265 | CA | PRO | B | 484 | 40.670 | 41.164 | 48.603 | 1.00 | 48.16 | C | |
| ATOM | 8267 | CB | PRO | B | 484 | 41.561 | 40.497 | 47.539 | 1.00 | 47.97 | C | |
| ATOM | 8270 | CG | PRO | B | 484 | 42.861 | 40.506 | 48.145 | 1.00 | 47.84 | C | |
| ATOM | 8273 | CD | PRO | B | 484 | 42.973 | 41.879 | 48.813 | 1.00 | 48.09 | C | |
| ATOM | 8276 | C | PRO | B | 484 | 40.040 | 40.051 | 49.414 | 1.00 | 47.96 | C | |
| ATOM | 8277 | O | PRO | B | 484 | 40.546 | 39.719 | 50.492 | 1.00 | 48.09 | O | |
| ATOM | 8278 | N | SER | B | 485 | 38.965 | 39.485 | 48.867 | 1.00 | 47.74 | N | |
| ATOM | 8279 | CA | SER | B | 485 | 38.386 | 38.228 | 49.351 | 1.00 | 47.65 | C | |
| ATOM | 8281 | CB | SER | B | 485 | 36.955 | 38.066 | 48.844 | 1.00 | 47.31 | C | |
| ATOM | 8284 | OC | SER | B | 485 | 36.897 | 37.963 | 47.424 | 1.00 | 46.10 | O | |
| ATOM | 8286 | C | SER | B | 485 | 39.222 | 37.055 | 48.839 | 1.00 | 48.37 | C | |
| ATOM | 8287 | O | SER | B | 485 | 39.935 | 37.184 | 47.856 | 1.00 | 48.58 | O | |
| ATOM | 8289 | N | PHE | B | 486 | 39.106 | 35.903 | 49.490 | 1.00 | 49.19 | N | |
| ATOM | 8290 | CA | PHE | B | 486 | 39.660 | 34.647 | 48.950 | 1.00 | 49.76 | C | |
| ATOM | 8292 | CB | PHE | B | 486 | 39.599 | 33.515 | 49.981 | 1.00 | 49.30 | C | |
| ATOM | 8295 | CG | PHE | B | 486 | 40.568 | 33.703 | 51.121 | 1.00 | 50.33 | C | |
| ATOM | 8296 | CD1 | PHE | B | 486 | 41.954 | 33.747 | 50.883 | 1.00 | 48.94 | C | |
| ATOM | 8298 | CE1 | PHE | B | 486 | 42.839 | 33.875 | 51.903 | 1.00 | 47.01 | C | |
| ATOM | 8300 | CZ | PHE | B | 486 | 42.382 | 33.986 | 53.195 | 1.00 | 48.38 | C | |
| ATOM | 8302 | CE2 | PHE | B | 486 | 41.028 | 33.983 | 53.462 | 1.00 | 48.35 | C | |
| ATOM | 8304 | CD2 | PHE | B | 486 40.124 | 33.834 | 52.430 | 1.00 | 50.31 | C | | |
| ATOM | 8306 | C | PHE | B | 486 | 39.044 | 34.192 | 47.628 | 1.00 | 50.28 | C | |
| ATOM | 8307 | O | PHE | B | 486 | 39.702 | 33.471 | 46.863 | 1.00 | 51.07 | O | |
| ATOM | 8309 | N | ALA | B | 487 | 37.806 | 34.588 | 47.341 | 1.00 | 50.36 | N | |
| ATOM | 8310 | CA | ALA | B | 487 | 37.247 | 34.302 | 46.016 | 1.00 | 50.69 | C | |
| ATOM | 8312 | CB | ALA | B | 487 | 35.755 | 34.530 | 46.003 | 1.00 | 50.39 | C | |
| ATOM | 8316 | C | ALA | B | 487 | 37.979 | 35.102 | 44.898 | 1.00 | 50.78 | C | |
| ATOM | 8317 | O | ALA | B | 487 | 38.420 | 34.493 | 43.928 | 1.00 | 49.54 | O | |
| ATOM | 8319 | N | GLU | B | 488 | 38.135 | 36.431 | 45.085 | 1.00 | 51.52 | N | |
| ATOM | 8320 | CA | GLU | B | 488 | 39.005 | 37.289 | 44.240 | 1.00 | 52.17 | C | |
| ATOM | 8322 | CB | GLU | B | 488 | 39.145 | 38.688 | 44.813 | 1.00 | 51.76 | C | |
| ATOM | 8325 | CG | GLU | B | 488 | 37.907 | 39.516 | 44.683 | 1.00 | 53.55 | C | |
| ATOM | 8328 | CD | GLU | B | 488 | 38.055 | 40.919 | 45.282 | 1.00 | 54.38 | C | |
| ATOM | 8329 | OE1 | GLU | B | 488 38.337 | 41.060 | 46.496 | 1.00 | 55.44 | O | | |
| ATOM | 8330 | OE2 | GLU | B | 488 | 37.839 | 41.891 | 44.537 | 1.00 | 57.21 | O | |
| ATOM | 8331 | C | GLU | B | 488 | 40.422 | 36.736 | 44.054 | 1.00 | 52.57 | C | |
| ATOM | 8332 | O | GLU | B | 488 | 40.920 | 36.674 | 42.921 | 1.00 | 52.92 | O | |
| ATOM | 8334 | N | ILE | B | 489 | 41.051 | 36.333 | 45.158 | 1.00 | 52.71 | N | |
| ATOM | 8335 | CA | ILE | B | 489 | 42.428 | 35.817 | 45.131 | 1.00 | 53.14 | C | |
| ATOM | 8337 | CB | ILE | B | 489 | 42.987 | 35.627 | 46.603 | 1.00 | 53.22 | C | |
| ATOM | 8339 | CG1 | ILE | B | 489 | 43.368 | 36.993 | 47.203 | 1.00 | 52.71 | C | |
| ATOM | 8342 | CD1 | ILE | B | 489 | 43.384 | 37.040 | 48.717 | 1.00 | 51.77 | C | |
| ATOM | 8346 | CG2 | ILE | B | 489 | 44.204 | 34.702 | 46.652 | 1.00 | 52.99 | C | |
| ATOM | 8350 | C | ILE | B | 489 | 42.562 | 34.534 | 44.263 | 1.00 | 53.84 | C | |
| ATOM | 8351 | O | ILE | B | 489 | 43.386 | 34.477 | 43.355 | 1.00 | 54.34 | O | |
| ATOM | 8353 | N | HIS | B | 490 | 41.751 | 33.528 | 44.549 | 1.00 | 54.21 | N | |
| ATOM | 8354 | CA | HIS | B | 490 | 41.764 | 32.273 | 43.810 | 1.00 | 54.31 | C | |
| ATOM | 8356 | CB | HIS | B | 490 | 40.716 | 31.326 | 44.397 | 1.00 | 55.03 | C | |
| ATOM | 8359 | CG | HIS | B | 490 | 40.501 | 30.087 | 43.597 | 1.00 | 54.84 | C | |
| ATOM | 8360 | ND1 | HIS | B | 490 | 41.315 | 28.977 | 43.714 | 1.00 | 56.14 | N | |
| ATOM | 8362 | CE1 | HIS | B | 490 | 40.889 | 28.039 | 42.890 | 1.00 | 57.22 | C | |
| ATOM | 8364 | NE2 | HIS | B | 490 | 39.842 | 28.508 | 42.234 | 1.00 | 57.57 | N | |
| ATOM | 8366 | CD2 | HIS | B | 490 | 39.582 | 29.790 | 42.658 | 1.00 | 54.81 | C | |
| ATOM | 8368 | C | HIS | B | 490 | 41.483 | 32.468 | 42.345 | 1.00 | 54.84 | C | |
| ATOM | 8369 | O | HIS | B | 490 | 42.070 | 31.800 | 41.533 | 1.00 | 55.20 | O | |
| ATOM | 8371 | N | GLN | B | 491 | 40.554 | 33.359 | 42.017 | 1.00 | 55.89 | N | |
| ATOM | 8372 | CA | GLN | B | 491 | 40.334 | 33.791 | 40.648 | 1.00 | 56.45 | C | |
| ATOM | 8374 | CB | GLN | B | 491 | 39.240 | 34.837 | 40.598 | 1.00 | 57.05 | C | |
| ATOM | 8377 | CG | GLN | B | 491 | 38.861 | 35.359 | 39.224 | 1.00 | 59.32 | C | |
| ATOM | 8380 | CD | GLN | B | 491 | 37.944 | 34.413 | 38.494 | 1.00 | 62.51 | C | |
| ATOM | 8381 | OE1 | GLN | B | 491 | 38.357 | 33.291 | 38.086 | 1.00 | 62.20 | O | |
| ATOM | 8382 | NE2 | GLN | B | 491 | 36.678 | 34.851 | 38.321 | 1.00 | 63.92 | N | |
| ATOM | 8385 | C | GLN | B | 491 | 41.625 | 34.362 | 40.082 | 1.00 | 56.93 | C | |
| ATOM | 8386 | O | GLN | B | 491 | 42.054 | 33.934 | 39.034 | 1.00 | 56.92 | O | |
| ATOM | 8388 | N | ALA | B | 492 | 42.277 | 35.291 | 40.772 | 1.00 | 57.65 | N | |
| ATOM | 8389 | CA | ALA | B | 492 | 43.565 | 35.795 | 40.265 | 1.00 | 58.86 | C | |
| ATOM | 8391 | CB | ALA | B | 492 | 44.256 | 36.723 | 41.272 | 1.00 | 58.37 | C | |
| ATOM | 8395 | C | ALA | B | 492 | 44.506 | 34.646 | 39.863 | 1.00 | 59.63 | C | |
| ATOM | 8396 | O | ALA | B | 492 | 45.083 | 34.693 | 38.783 | 1.00 | 60.12 | O | |
| ATOM | 8398 | N | PHE | B | 493 | 44.596 | 33.612 | 40.715 | 1.00 | 60.62 | N | |
| ATOM | 8399 | CA | PHE | B | 493 | 45.524 | 32.474 | 40.544 | 1.00 | 61.20 | C | |
| ATOM | 8401 | CB | PHE | B | 493 | 45.811 | 31.756 | 41.872 | 1.00 | 60.84 | C | |
| ATOM | 8404 | CG | PHE | B | 493 | 46.787 | 32.481 | 42.736 | 1.00 | 60.62 | C | |
| ATOM | 8405 | CD1 | PHE | B | 493 | 48.144 | 32.458 | 42.438 | 1.00 | 60.75 | C | |
| ATOM | 8407 | CE1 | PHE | B | 493 | 49.063 | 33.153 | 43.247 | 1.00 | 60.89 | C | |
| ATOM | 8409 | CZ | PHE | B | 493 | 48.614 | 33.853 | 44.357 | 1.00 | 59.58 | C | |
| ATOM | 8411 | CE2 | PHE | B | 493 | 47.270 | 33.854 | 44.653 | 1.00 | 58.93 | C | |
| ATOM | 8413 | CD2 | PHE | B | 493 | 46.367 | 33.198 | 43.841 | 1.00 | 59.95 | C | |
| ATOM | 8415 | C | PHE | B | 493 | 45.095 | 31.444 | 39.520 | 1.00 | 63.06 | C | |
| ATOM | 8416 | O | PHE | B | 493 | 45.962 | 30.859 | 38.849 | 1.00 | 63.12 | O | |
| ATOM | 8418 | N | GLU | B | 494 | 43.782 | 31.191 | 39.433 | 1.00 | 64.65 | N | |
| ATOM | 8419 | CA | GLU | B | 494 | 43.219 | 30.444 | 38.314 | 1.00 | 65.94 | C | |
| ATOM | 8421 | CB | GLU | B | 494 | 41.690 | 30.316 | 38.405 | 1.00 | 65.82 | C | |
| ATOM | 8424 | CG | GLU | B | 494 | 41.198 | 28.897 | 38.755 | 1.00 | 67.78 | C | |
| ATOM | 8427 | CD | GLU | B | 494 | 39.682 | 28.697 | 38.542 | 1.00 | 68.60 | C | |
| ATOM | 8428 | OE1 | GLU | B | 494 | 39.075 | 29.320 | 37.621 | 1.00 | 69.70 | O | |
| ATOM | 8429 | OE2 | GLU | B | 494 | 39.080 | 27.899 | 39.305 | 1.00 | 72.77 | O | |
| ATOM | 8430 | C | GLU | B | 494 | 43.698 | 31.046 | 36.963 | 1.00 | 65.78 | C | |
| ATOM | 8431 | O | GLU | B | 494 | 44.485 | 30.390 | 36.285 | 1.00 | 66.61 | O | |
| ATOM | 8433 | N | THR | B | 495 | 43.305 | 32.281 | 36.638 | 1.00 | 65.70 | N | |
| ATOM | 8434 | CA | THR | B | 495 | 43.771 | 33.022 | 35.445 | 1.00 | 66.27 | C | |
| ATOM | 8436 | CB | THR | B | 495 | 43.591 | 34.550 | 35.668 | 1.00 | 66.55 | C | |
| ATOM | 8438 | OG1 | THR | B | 495 | 42.235 | 34.865 | 36.035 | 1.00 | 67.15 | O | |
| ATOM | 8440 | CG2 | THR | B | 495 | 43.865 | 35.370 | 34.391 | 1.00 | 66.40 | C | |
| ATOM | 8444 | C | THR | B | 495 | 45.253 | 32.777 | 35.097 | 1.00 | 67.37 | C | |
| ATOM | 8445 | O | THR | B | 495 | 45.593 | 32.295 | 33.992 | 1.00 | 68.13 | O | |
| ATOM | 8447 | N | MET | B | 496 | 46.113 | 33.062 | 36.081 | 1.00 | 68.02 | N | |
| ATOM | 8448 | CA | MET | B | 496 | 47.569 | 32.920 | 35.961 | 1.00 | 67.97 | C | |
| ATOM | 8450 | CB | MET | B | 496 | 48.274 | 33.481 | 37.192 | 1.00 | 67.57 | C | |
| ATOM | 8453 | CG | MET | B | 496 | 48.155 | 34.951 | 37.332 | 1.00 | 65.98 | C | |
| ATOM | 8456 | SD | MET | B | 496 | 49.115 | 35.559 | 38.741 | 1.00 | 65.69 | S | |
| ATOM | 8457 | CE | MET | B | 496 | 48.124 | 35.222 | 40.160 | 1.00 | 62.73 | C | |
| ATOM | 8461 | C | MET | B | 496 | 48.018 | 31.471 | 35.754 | 1.00 | 68.66 | C | |
| ATOM | 8462 | O | MET | B | 496 | 48.618 | 31.199 | 34.750 | 1.00 | 69.04 | O | |
| ATOM | 8464 | N | PHE | B | 497 | 47.716 | 30.547 | 36.673 | 1.00 | 69.72 | N | |
| ATOM | 8465 | CA | PHE | B | 497 | 48.080 | 29.094 | 36.512 | 1.00 | 70.34 | C | |
| ATOM | 8467 | CB | PHE | B | 497 | 47.719 | 28.262 | 37.806 | 1.00 | 70.36 | C | |
| ATOM | 8470 | CG | PHE | B | 497 | 47.975 | 26.747 | 37.700 | 1.00 | 70.15 | C | |
| ATOM | 8471 | CD1 | PHE | B | 497 | 49.278 | 26.237 | 37.610 | 1.00 | 70.56 | C | |
| ATOM | 8473 | CE1 | PHE | B | 497 | 49.515 | 24.808 | 37.482 | 1.00 | 70.09 | C | |
| ATOM | 8475 | CZ | PHE | B | 497 | 48.439 | 23.906 | 37.448 | 1.00 | 69.28 | C | |
| ATOM | 8477 | CE2 | PHE | B | 497 | 47.129 | 24.400 | 37.545 | 1.00 | 69.73 | C | |
| ATOM | 8479 | CD2 | PHE | B | 497 | 46.902 | 25.825 | 37.670 | 1.00 | 71.15 | C | |
| ATOM | 8481 | C | PHE | B | 497 | 47.540 | 28.450 | 35.169 | 1.00 | 71.18 | C | |
| ATOM | 8482 | O | PHE | B | 497 | 47.983 | 27.338 | 34.786 | 1.00 | 71.42 | O | |
| ATOM | 8484 | N | GLN | B | 498 | 46.615 | 29.140 | 34.469 | 1.00 | 71.40 | N | |
| ATOM | 8485 | CA | GLN | B | 498 | 46.359 | 28.898 | 33.037 | 1.00 | 71.65 | C | |
| ATOM | 8487 | CB | GLN | B | 498 | 44.851 | 28.828 | 32.725 | 1.00 | 72.74 | C | |
| ATOM | 8490 | CG | GLN | B | 498 | 43.847 | 28.239 | 33.798 | 1.00 | 74.51 | C | |
| ATOM | 8493 | CD | GLN | B | 498 | 44.388 | 27.082 | 34.648 | 1.00 | 77.50 | C | |
| ATOM | 8494 | OE1 | GLN | B | 498 | 45.270 | 26.323 | 34.203 | 1.00 | 79.28 | O | |
| ATOM | 8495 | NE2 | GLN | B | 498 | 43.854 | 26.947 | 35.882 | 1.00 | 77.78 | N | |
| ATOM | 8498 | C | GLN | B | 498 | 47.014 | 29.997 | 32.193 | 1.00 | 71.23 | C | |
| ATOM | 8499 | O | GLN | B | 498 | 48.215 | 29.953 | 31.932 | 1.00 | 70.66 | O | |
| ATOM | 8501 | N | LYS | C | 234 | 51.161 | 5.079 | −3.312 | 1.00 | 84.86 | N | |
| ATOM | 8502 | CA | LYS | C | 234 | 50.027 | 4.587 | −4.152 | 1.00 | 84.92 | C | |
| ATOM | 8504 | CB | LYS | C | 234 | 50.371 | 4.575 | −5.645 | 1.00 | 84.78 | C | |
| ATOM | 8507 | CG | LYS | C | 234 | 50.545 | 5.970 | −6.208 | 1.00 | 84.60 | C | |
| ATOM | 8510 | CD | LYS | C | 234 | 50.956 | 5.950 | −7.682 | 1.00 | 84.78 | C | |
| ATOM | 8513 | CE | LYS | C | 234 | 51.581 | 7.283 | −8.093 | 1.00 | 84.70 | C | |
| ATOM | 8516 | NZ | LYS | C | 234 | 51.871 | 7.382 | −9.548 | 1.00 | 84.28 | N | |
| ATOM | 8520 | C | LYS | C | 234 | 49.515 | 3.216 | −3.700 | 1.00 | 85.24 | C | |
| ATOM | 8521 | O | LYS | C | 234 | 50.263 | 2.365 | −3.200 | 1.00 | 84.78 | O | |
| ATOM | 8525 | N | TRP | C | 235 | 48.224 | 3.016 | −3.958 | 1.00 | 85.50 | N | |
| ATOM | 8526 | CA | TRP | C | 235 | 47.381 | 2.035 | −3.268 | 1.00 | 85.45 | C | |
| ATOM | 8528 | CB | TRP | C | 235 | 45.952 | 2.617 | −3.092 | 1.00 | 84.79 | C | |
| ATOM | 8531 | CG | TRP | C | 235 | 45.869 | 3.990 | −2.438 | 1.00 | 83.98 | C | |
| ATOM | 8532 | CD1 | TRP | C | 235 | 46.366 | 5.193 | −2.912 | 1.00 | 83.64 | C | |
| ATOM | 8534 | NE1 | TRP | C | 235 | 46.083 | 6.211 | −2.035 | 1.00 | 83.11 | N | |
| ATOM | 8536 | CE2 | TRP | C | 235 | 45.389 | 5.693 | −0.979 | 1.00 | 82.49 | C | |
| ATOM | 8537 | CD2 | TRP | C | 235 | 45.232 | 4.293 | −1.209 | 1.00 | 82.73 | C | |
| ATOM | 8538 | CE3 | TRP | C | 235 | 44.547 | 3.524 | −0.256 | 1.00 | 82.49 | C | |
| ATOM | 8540 | CZ3 | TRP | C | 235 | 44.046 | 4.153 | 0.870 | 1.00 | 83.44 | C | |
| ATOM | 8542 | CH2 | TRP | C | 235 | 44.206 | 5.550 | 1.060 | 1.00 | 84.32 | C | |
| ATOM | 8544 | CZ2 | TRP | C | 235 | 44.883 | 6.328 | 0.145 | 1.00 | 83.50 | C | |
| ATOM | 8546 | C | TRP | C | 235 | 47.322 | 0.719 | −4.064 | 1.00 | 86.06 | C | |
| ATOM | 8547 | O | TRP | C | 235 | 46.552 | −0.210 | −3.737 | 1.00 | 86.09 | O | |
| ATOM | 8549 | N | GLU | C | 236 | 48.166 | 0.629 | −5.086 | 1.00 | 86.66 | N | |
| ATOM | 8550 | CA | GLU | C | 236 | 47.990 | −0.326 | −6.142 | 1.00 | 87.32 | C | |
| ATOM | 8552 | CB | GLU | C | 236 | 48.817 | 0.068 | −7.355 | 1.00 | 87.50 | C | |
| ATOM | 8555 | CG | GLU | C | 236 | 48.494 | 1.443 | −7.921 | 1.00 | 88.15 | C | |
| ATOM | 8558 | CD | GLU | C | 236 | 47.187 | 1.514 | −8.696 | 1.00 | 88.82 | C | |
| ATOM | 8559 | OE1 | GLU | C | 236 | 46.855 | 0.570 | −9.457 | 1.00 | 87.60 | O | |
| ATOM | 8560 | OE2 | GLU | C | 236 | 46.509 | 2.556 | −8.571 | 1.00 | 90.25 | O | |
| ATOM | 8561 | C | GLU | C | 236 | 48.490 | −1.618 | −5.585 | 1.00 | 87.71 | C | |
| ATOM | 8562 | O | GLU | C | 236 | 49.439 | −1.609 | −4.823 | 1.00 | 87.64 | O | |
| ATOM | 8564 | N | MET | C | 237 | 47.822 | −2.710 | −5.922 | 1.00 | 88.54 | N | |
| ATOM | 8565 | CA | MET | C | 237 | 48.209 | −4.032 | −5.447 | 1.00 | 89.45 | C | |
| ATOM | 8567 | CB | MET | C | 237 | 47.386 | −4.415 | −4.218 | 1.00 | 89.64 | C | |
| ATOM | 8570 | CG | MET | C | 237 | 45.857 | −4.382 | −4.406 | 1.00 | 89.90 | C | |
| ATOM | 8573 | SD | MET | C | 237 | 45.033 | −5.712 | −3.492 | 1.00 | 90.93 | S | |
| ATOM | 8574 | CE | MET | C | 237 | 45.433 | −7.061 | −4.384 | 1.00 | 90.25 | C | |
| ATOM | 8578 | C | MET | C | 237 | 48.009 | −5.048 | −6.528 | 1.00 | 89.63 | C | |
| ATOM | 8579 | O | MET | C | 237 | 47.304 | −4.783 | −7.487 | 1.00 | 89.93 | O | |
| ATOM | 8581 | N | GLU | C | 238 | 48.590 | −6.223 | −6.331 | 1.00 | 90.33 | N | |
| ATOM | 8582 | CA | GLU | C | 238 | 48.585 | −7.291 | −7.333 | 1.00 | 90.87 | C | |
| ATOM | 8584 | CB | GLU | C | 238 | 49.825 | −8.188 | −7.129 | 1.00 | 90.62 | C | |
| ATOM | 8587 | CG | GLU | C | 238 | 49.892 | −9.462 | −7.957 | 1.00 | 90.54 | C | |
| ATOM | 8590 | CD | GLU | C | 238 | 49.932 | −9.205 | −9.453 | 1.00 | 90.68 | C | |
| ATOM | 8591 | OE1 | GLU | C | 238 | 48.896 | −8.788 | −10.040 | 1.00 | 92.10 | O | |
| ATOM | 8592 | OE2 | GLU | C | 238 | 51.006 | −9.429 | −10.046 | 1.00 | 88.72 | O | |
| ATOM | 8593 | C | GLU | C | 238 | 47.302 | −8.097 | −7.171 | 1.00 | 91.22 | C | |
| ATOM | 8594 | O | GLU | C | 238 | 47.096 | −8.642 | −6.092 | 1.00 | 91.62 | O | |
| ATOM | 8596 | N | ARG | C | 239 | 46.479 | −8.229 | −8.221 | 1.00 | 91.41 | N | |
| ATOM | 8597 | CA | ARG | C | 239 | 45.197 | −8.969 | −8.097 | 1.00 | 91.90 | C | |
| ATOM | 8599 | CB | ARG | C | 239 | 44.330 | −8.842 | −9.358 | 1.00 | 91.80 | C | |
| ATOM | 8602 | CG | ARG | C | 239 | 44.650 | −9.812 | −10.504 | 1.00 | 91.37 | C | |
| ATOM | 8605 | CD | ARG | C | 239 | 43.938 | −9.499 | −11.809 | 1.00 | 91.13 | C | |
| ATOM | 8608 | NE | ARG | C | 239 | 44.486 | −8.314 | −12.483 | 1.00 | 90.38 | N | |
| ATOM | 8610 | CZ | ARG | C | 239 | 44.040 | −7.045 | −12.398 | 1.00 | 89.51 | C | |
| ATOM | 8611 | NH1 | ARG | C | 239 | 42.993 | −6.708 | −11.645 | 1.00 | 88.88 | N | |
| ATOM | 8614 | NH2 | ARG | C | 239 | 44.668 | −6.086 | −13.088 | 1.00 | 89.27 | N | |
| ATOM | 8617 | C | ARG | C | 239 | 45.336 | −10.459 | −7.666 | 1.00 | 92.76 | C | |
| ATOM | 8618 | O | ARG | C | 239 | 44.423 | −10.983 | −7.017 | 1.00 | 93.07 | O | |
| ATOM | 8620 | N | THR | C | 240 | 46.456 | −11.110 | −8.032 | 1.00 | 93.35 | N | |
| ATOM | 8621 | CA | THR | C | 240 | 46.890 | −12.431 | −7.489 | 1.00 | 93.82 | C | |
| ATOM | 8623 | CB | THR | C | 240 | 48.349 | −12.753 | −7.912 | 1.00 | 93.80 | C | |
| ATOM | 8625 | OG1 | THR | C | 240 | 48.529 | −12.458 | −9.302 | 1.00 | 93.48 | O | |
| ATOM | 8627 | CG2 | THR | C | 240 | 48.678 | −14.266 | −7.766 | 1.00 | 93.29 | C | |
| ATOM | 8631 | C | THR | C | 240 | 46.849 | −12.587 | −5.962 | 1.00 | 94.63 | C | |
| ATOM | 8632 | O | THR | C | 240 | 46.531 | −13.682 | −5.464 | 1.00 | 95.01 | O | |
| ATOM | 8634 | N | ASP | C | 241 | 47.217 | −11.520 | −5.236 | 1.00 | 95.30 | N | |
| ATOM | 8635 | CA | ASP | C | 241 | 47.161 | −11.491 | −3.745 | 1.00 | 95.66 | C | |
| ATOM | 8637 | CB | ASP | C | 241 | 47.511 | −10.103 | −3.171 | 1.00 | 95.85 | C | |
| ATOM | 8640 | CG | ASP | C | 241 | 48.958 | −9.702 | −3.402 | 1.00 | 96.55 | C | |
| ATOM | 8641 | OD1 | ASP | C | 241 | 49.661 | −10.326 | 4.240 | 1.00 | 97.52 | O | |
| ATOM | 8642 | OD2 | ASP | C | 241 | 49.468 | −8.745 | −2.779 | 1.00 | 97.25 | O | |
| ATOM | 8643 | C | ASP | C | 241 | 45.800 | −11.868 | −3.181 | 1.00 | 96.34 | C | |
| ATOM | 8644 | O | ASP | C | 241 | 45.731 | −12.486 | −2.122 | 1.00 | 96.64 | O | |
| ATOM | 8646 | N | ILE | C | 242 | 44.739 | −11.471 | −3.891 | 1.00 | 97.13 | N | |
| ATOM | 8647 | CA | ILE | C | 242 | 43.357 | −11.621 | −3.449 | 1.00 | 97.68 | C | |
| ATOM | 8649 | CB | ILE | C | 242 | 42.579 | −10.273 | −3.727 | 1.00 | 97.53 | C | |
| ATOM | 8651 | CG1 | ILE | C | 242 | 42.828 | −9.326 | −2.539 | 1.00 | 97.60 | C | |
| ATOM | 8654 | CD1 | ILE | C | 242 | 42.359 | −7.914 | −2.715 | 1.00 | 97.66 | C | |
| ATOM | 8658 | CG2 | ILE | C | 242 | 41.070 | −10.486 | −3.975 | 1.00 | 97.64 | C | |
| ATOM | 8662 | C | ILE | C | 242 | 42.688 | −12.892 | −4.035 | 1.00 | 98.04 | C | |
| ATOM | 8663 | O | ILE | C | 242 | 42.739 | −13.128 | −5.251 | 1.00 | 98.30 | O | |
| ATOM | 8665 | N | THR | C | 243 | 42.086 | −13.698 | −3.144 | 1.00 | 98.38 | N | |
| ATOM | 8666 | CA | THR | C | 243 | 41.250 | −14.847 | −3.508 | 1.00 | 98.44 | C | |
| ATOM | 8668 | CB | THR | C | 243 | 41.141 | −15.895 | −2.322 | 1.00 | 98.54 | C | |
| ATOM | 8670 | OG1 | THR | C | 243 | 42.442 | −16.311 | −1.868 | 1.00 | 97.80 | O | |
| ATOM | 8672 | CG2 | THR | C | 243 | 40.488 | −17.202 | 2.780 | 1.00 | 98.33 | C | |
| ATOM | 8676 | C | THR | C | 243 | 39.867 | −14.288 | −3.841 | 1.00 | 98.81 | C | |
| ATOM | 8677 | O | THR | C | 243 | 39.232 | −13.673 | −2.981 | 1.00 | 98.69 | O | |
| ATOM | 8679 | N | MET | C | 244 | 39.417 | −14.475 | −5.080 | 1.00 | 99.22 | N | |
| ATOM | 8680 | CA | MET | C | 244 | 38.030 | −14.169 | −5.444 | 1.00 | 99.77 | C | |
| ATOM | 8682 | CB | MET | C | 244 | 37.907 | −13.907 | −6.950 | 1.00 | 100.08 | C | |
| ATOM | 8685 | CG | MET | C | 244 | 38.687 | −12.680 | −7.415 | 1.00 | 100.91 | C | |
| ATOM | 8688 | SD | MET | C | 244 | 38.463 | −11.154 | −6.350 | 1.00 | 103.39 | S | |
| ATOM | 8689 | CE | MET | C | 244 | 36.662 | −10.947 | −6.325 | 1.00 | 102.89 | C | |
| ATOM | 8693 | C | MET | C | 244 | 37.116 | −15.304 | −4.992 | 1.00 | 99.81 | C | |
| ATOM | 8694 | O | MET | C | 244 | 37.443 | −16.478 | −5.213 | 1.00 | 99.88 | O | |
| ATOM | 8696 | N | LYS | C | 245 | 35.991 | −14.943 | −4.351 | 1.00 | 99.88 | N | |
| ATOM | 8697 | CA | LYS | C | 245 | 35.077 | −15.899 | −3.684 | 1.00 | 99.95 | C | |
| ATOM | 8699 | CB | LYS | C | 245 | 35.139 | −15.733 | −2.163 | 1.00 | 99.90 | C | |
| ATOM | 8702 | CG | LYS | C | 245 | 36.526 | −15.978 | 1.559 | 1.00 | 99.53 | C | |
| ATOM | 8705 | CD | LYS | C | 245 | 36.455 | −16.198 | −0.025 | 1.00 | 99.67 | C | |
| ATOM | 8708 | CE | LYS | C | 245 | 37.304 | −17.390 | 0.454 | 1.00 | 98.83 | C | |
| ATOM | 8711 | NZ | LYS | C | 245 | 37.046 | −17.716 | 1.894 | 1.00 | 98.04 | N | |
| ATOM | 8715 | C | LYS | C | 245 | 33.603 | −15.885 | −4.190 | 1.00 | 100.18 | C | |
| ATOM | 8716 | O | LYS | C | 245 | 33.170 | −16.886 | −4.793 | 1.00 | 100.45 | O | |
| ATOM | 8718 | N | HIS | C | 246 | 32.832 | −14.811 | −3.936 | 1.00 | 100.00 | N | |
| ATOM | 8719 | CA | HIS | C | 246 | 31.441 | −14.722 | −4.456 | 1.00 | 99.79 | C | |
| ATOM | 8721 | CB | HIS | C | 246 | 30.484 | −15.648 | −3.673 | 1.00 | 99.90 | C | |
| ATOM | 8724 | CG | HIS | C | 246 | 30.236 | −15.239 | −2.252 | 1.00 | 100.29 | C | |
| ATOM | 8725 | ND1 | HIS | C | 246 | 29.176 | −14.436 | 1.886 | 1.00 | 100.70 | N | |
| ATOM | 8727 | CE1 | HIS | C | 246 | 29.187 | −14.262 | 0.576 | 1.00 | 100.31 | C | |
| ATOM | 8729 | NE2 | HIS | C | 246 | 30.210 | −14.933 | −0.076 | 1.00 | 100.44 | N | |
| ATOM | 8731 | CD2 | HIS | C | 246 | 30.876 | −15.562 | 1.102 | 1.00100.68 | C | | |
| ATOM | 8733 | C | HIS | C | 246 | 30.827 | −13.321 | −4.569 | 1.00 | 99.47 | C | |
| ATOM | 8734 | O | HIS | C | 246 | 31.271 | −12.390 | −3.915 | 1.00 | 99.03 | O | |
| ATOM | 8736 | N | LYS | C | 247 | 29.802 | −13.228 | −5.427 | 1.00 | 99.43 | N | |
| ATOM | 8737 | CA | LYS | C | 247 | 28.957 | −12.017 | −5.661 | 1.00 | 99.45 | C | |
| ATOM | 8739 | CB | LYS | C | 247 | 27.947 | −12.289 | −6.822 | 1.00 | 99.46 | C | |
| ATOM | 8742 | CG | LYS | C | 247 | 26.634 | −11.451 | −6.936 | 1.00 | 99.38 | C | |
| ATOM | 8745 | CD | LYS | C | 247 | 25.562 | −12.201 | −7.758 | 1.00 | 99.19 | C | |
| ATOM | 8748 | CE | LYS | C | 247 | 24.199 | −11.546 | −7.634 | 1.00 | 98.82 | C | |
| ATOM | 8751 | NZ | LYS | C | 247 | 24.245 | −10.137 | −8.111 | 1.00 | 98.80 | N | |
| ATOM | 8755 | C | LYS | C | 247 | 28.239 | −11.559 | −4.384 | 1.00 | 99.32 | C | |
| ATOM | 8756 | O | LYS | C | 247 | 27.975 | −12.357 | −3.483 | 1.00 | 99.35 | O | |
| ATOM | 8758 | N | LEU | C | 248 | 27.922 | −10.268 | −4.329 | 1.00 | 99.34 | N | |
| ATOM | 8759 | CA | LEU | C | 248 | 27.407 | −9.629 | −3.114 | 1.00 | 99.22 | C | |
| ATOM | 8761 | CB | LEU | C | 248 | 28.536 | −9.560 | −2.059 | 1.00 | 99.35 | C | |
| ATOM | 8764 | CG | LEU | C | 248 | 28.159 | −9.351 | −0.580 | 1.00 | 99.81 | C | |
| ATOM | 8766 | CD1 | LEU | C | 248 | 29.043 | −10.214 | 0.371 | 1.00 | 99.31 | C | |
| ATOM | 8770 | CD2 | LEU | C | 248 | 28.172 | −7.838 | −0.173 | 1.00 | 100.61 | C | |
| ATOM | 8774 | C | LEU | C | 248 | 26.857 | −8.218 | −3.407 | 1.00 | 99.06 | C | |
| ATOM | 8775 | O | LEU | C | 248 | 25.662 | −7.946 | −3.277 | 1.00 | 98.76 | O | |
| ATOM | 8777 | N | GLY | C | 254 | 24.384 | −6.467 | −7.591 | 1.00 | 108.36 | N | |
| ATOM | 8778 | CA | GLY | C | 254 | 25.451 | −5.599 | −8.104 | 1.00 | 108.51 | C | |
| ATOM | 8781 | C | GLY | C | 254 | 26.846 | −6.211 | −8.142 | 1.00 | 108.59 | C | |
| ATOM | 8782 | O | GLY | C | 254 | 27.044 | −7.356 | −7.686 | 1.00 | 108.70 | O | |
| ATOM | 8784 | N | GLU | C | 255 | 27.812 | −5.418 | −8.639 | 1.00 | 108.45 | N | |
| ATOM | 8785 | CA | GLU | C | 255 | 29.218 | −5.854 | −8.911 | 1.00 | 108.34 | C | |
| ATOM | 8787 | CB | GLU | C | 255 | 29.758 | −5.083 | −10.140 | 1.00 | 108.26 | C | |
| ATOM | 8790 | CG | GLU | C | 255 | 28.939 | −5.286 | −11.415 | 1.00 | 109.15 | C | |
| ATOM | 8793 | CD | GLU | C | 255 | 28.803 | −6.756 | −11.842 | 1.00 | 110.72 | C | |
| ATOM | 8794 | OE1 | GLU | C | 255 | 29.692 | −7.591 | −11.506 | 1.00 | 110.51 | O | |
| ATOM | 8795 | OE2 | GLU | C | 255 | 27.793 | −7.085 | −12.515 | 1.00 | 111.23 | O | |
| ATOM | 8796 | C | GLU | C | 255 | 30.227 | −5.811 | −7.714 | 1.00 | 107.99 | C | |
| ATOM | 8797 | O | GLU | C | 255 | 31.435 | −5.555 | −7.877 | 1.00 | 107.25 | O | |
| ATOM | 8799 | N | VAL | C | 256 | 29.703 | −6.122 | −6.526 | 1.00 | 107.88 | N | |
| ATOM | 8800 | CA | VAL | C | 256 | 30.454 | −6.183 | −5.272 | 1.00 | 107.97 | C | |
| ATOM | 8802 | CB | VAL | C | 256 | 29.633 | −5.545 | −4.096 | 1.00 | 107.99 | C | |
| ATOM | 8804 | CG1 | VAL | C | 256 | 30.308 | −5.763 | −2.713 | 1.00 | 108.35 | C | |
| ATOM | 8808 | CG2 | VAL | C | 256 | 29.400 | −4.045 | −4.348 | 1.00 | 108.16 | C | |
| ATOM | 8812 | C | VAL | C | 256 | 30.768 | −7.671 | −5.004 | 1.00 | 107.74 | C | |
| ATOM | 8813 | O | VAL | C | 256 | 29.921 | −8.554 | −5.256 | 1.00 | 107.77 | O | |
| ATOM | 8815 | N | TYR | C | 257 | 31.983 | −7.934 | −4.514 | 1.00 | 107.19 | N | |
| ATOM | 8816 | CA | TYR | C | 257 | 32.472 | −9.292 | −4.302 | 1.00 | 106.96 | C | |
| ATOM | 8818 | CB | TYR | C | 257 | 33.331 | −9.729 | −5.513 | 1.00 | 107.49 | C | |
| ATOM | 8821 | CG | TYR | C | 257 | 32.518 | −10.065 | −6.777 | 1.00 | 108.24 | C | |
| ATOM | 8822 | CD | TYR | C | 257 | 32.095 | −9.049 | −7.668 | 1.00 | 108.98 | C | |
| ATOM | 8824 | CE1 | TYR | C | 257 | 31.328 | −9.353 | −8.833 | 1.00 | 108.46 | C | |
| ATOM | 8826 | CZ | TYR | C | 257 | 30.997 | −10.681 | −9.111 | 1.00 | 108.09 | C | |
| ATOM | 8827 | OH | TYR | C | 257 | 30.263 | −10.958 | −10.234 | 1.00 | 108.03 | O | |
| ATOM | 8829 | CE2 | TYR | C | 257 | 31.406 | −11.710 | −8.253 | 1.00 | 108.05 | C | |
| ATOM | 8831 | CD2 | TYR | C | 257 | 32.166 | −11.400 | −7.089 | 1.00 | 108.45 | C | |
| ATOM | 8833 | C | TYR | C | 257 | 33.252 | −9.402 | −2.979 | 1.00 | 106.19 | C | |
| ATOM | 8834 | O | TYR | C | 257 | 34.246 | −8.701 | −2.790 | 1.00 | 105.86 | O | |
| ATOM | 8836 | N | GLU | C | 258 | 32.784 | −10.259 | −2.062 | 1.00 | 105.32 | N | |
| ATOM | 8837 | CA | GLU | C | 258 | 33.590 | −10.648 | −0.907 | 1.00 | 104.86 | C | |
| ATOM | 8839 | CB | GLU | C | 258 | 32.860 | −11.570 | 0.080 | 1.00 | 104.83 | C | |
| ATOM | 8842 | CG | GLU | C | 258 | 33.647 | −11.806 | 1.379 | 1.00 | 104.93 | C | |
| ATOM | 8845 | CD | GLU | C | 258 | 33.613 | −13.241 | 1.910 | 1.00 | 104.95 | C | |
| ATOM | 8846 | OE1 | GLU | C | 258 | 33.985 | −14.192 | 1.188 | 1.00 | 105.36 | O | |
| ATOM | 8847 | OE2 | GLU | C | 258 | 33.249 | −13.421 | 3.085 | 1.00 | 104.39 | O | |
| ATOM | 8848 | C | GLU | C | 258 | 34.774 | −11.397 | −1.471 | 1.00 | 104.59 | C | |
| ATOM | 8849 | O | GLU | C | 258 | 34.597 | −12.419 | −2.137 | 1.00 | 104.59 | O | |
| ATOM | 8851 | N | GLY | C | 259 | 35.959 | −10.841 | −1.265 | 1.00 | 104.01 | N | |
| ATOM | 8852 | CA | GLY | C | 259 | 37.198 | −11.559 | −1.483 | 1.00 | 103.54 | C | |
| ATOM | 8855 | C | GLY | C | 259 | 37.780 | −11.997 | −0.157 | 1.00 | 103.14 | C | |
| ATOM | 8856 | O | GLY | C | 259 | 37.152 | −11.880 | 0.902 | 1.00 | 103.00 | O | |
| ATOM | 8858 | N | VAL | C | 260 | 38.976 | −12.562 | −0.251 | 1.00 | 102.66 | N | |
| ATOM | 8859 | CA | VAL | C | 260 | 39.933 | −12.614 | 0.852 | 1.00 | 102.10 | C | |
| ATOM | 8861 | CB | VAL | C | 260 | 40.083 | −14.086 | 1.401 | 1.00 | 102.14 | C | |
| ATOM | 8863 | CG1 | VAL | C | 260 | 41.466 | −14.380 | 2.010 | 1.00 | 101.62 | C | |
| ATOM | 8867 | CG2 | VAL | C | 260 | 38.974 | −14.371 | 2.424 | 1.00 | 101.91 | C | |
| ATOM | 8871 | C | VAL | C | 260 | 41.224 | −11.982 | 0.301 | 1.00 | 101.87 | C | |
| ATOM | 8872 | O | VAL | C | 260 | 41.472 | −12.057 | −0.901 | 1.00 | 101.80 | O | |
| ATOM | 8874 | N | TRP | C | 261 | 41.979 | −11.306 | 1.172 | 1.00 | 101.46 | N | |
| ATOM | 8875 | CA | TRP | C | 261 | 43.363 | −10.883 | 0.906 | 1.00 | 101.15 | C | |
| ATOM | 8877 | CB | TRP | C | 261 | 43.607 | −9.452 | 1.424 | 1.00 | 100.56 | C | |
| ATOM | 8880 | CG | TRP | C | 261 | 44.820 | −8.751 | 0.859 | 1.00 | 100.08 | C | |
| ATOM | 8881 | CD1 | TRP | C | 261 | 46.019 | −9.314 | 0.508 | 1.00 | 99.76 | C | |
| ATOM | 8883 | NE1 | TRP | C | 261 | 46.878 | −8.349 | 0.038 | 1.00 | 99.88 | N | |
| ATOM | 8885 | CE2 | TRP | C | 261 | 46.257 | −7.128 | 0.090 | 1.00 | 99.91 | C | |
| ATOM | 8886 | CD2 | TRP | C | 261 | 44.954 | −7.343 | 0.609 | 1.00 | 100.10 | C | |
| ATOM | 8887 | CE3 | TRP | C | 261 | 44.095 | −6.230 | 0.755 | 1.00 | 99.78 | C | |
| ATOM | 8889 | CZ3 | TRP | C | 261 | 44.568 | −4.948 | 0.385 | 1.00 | 99.66 | C | |
| ATOM | 8891 | CH2 | TRP | C | 261 | 45.877 | −4.776 | −0.121 | 1.00 | 99.48 | C | |
| ATOM | 8893 | CZ2 | TRP | C | 261 | 46.731 | −5.844 | −0.276 | 1.00 | 99.65 | C | |
| ATOM | 8895 | C | TRP | C | 261 | 44.226 | −11.944 | 1.612 | 1.00 | 101.19 | C | |
| ATOM | 8896 | O | TRP | C | 261 | 44.649 | −11.759 | 2.763 | 1.00 | 101.16 | O | |
| ATOM | 8898 | N | LYS | C | 262 | 44.470 | −13.051 | 0.892 | 1.00 | 101.20 | N | |
| ATOM | 8899 | CA | LYS | C | 262 | 45.018 | −14.337 | 1.437 | 1.00 | 100.96 | C | |
| ATOM | 8901 | CB | LYS | C | 262 | 45.228 | −15.345 | 0.272 | 1.00 | 100.90 | C | |
| ATOM | 8904 | CG | LYS | C | 262 | 45.455 | −16.811 | 0.659 | 1.00 | 100.96 | C | |
| ATOM | 8907 | CD | LYS | C | 262 | 44.226 | −17.514 | 1.252 | 1.00 | 100.64 | C | |
| ATOM | 8910 | CE | LYS | C | 262 | 44.633 | −18.893 | 1.816 | 1.00 | 100.87 | C | |
| ATOM | 8913 | NZ | LYS | C | 262 | 43.535 | −19.675 | 2.475 | 1.00 | 100.56 | N | |
| ATOM | 8917 | C | LYS | C | 262 | 46.296 | −14.217 | 2.320 | 1.00 | 100.80 | C | |
| ATOM | 8918 | O | LYS | C | 262 | 46.505 | −15.024 | 3.228 | 1.00 | 100.85 | O | |
| ATOM | 8920 | N | LYS | C | 263 | 47.124 | −13.211 | 2.028 | 1.00 | 100.60 | N | |
| ATOM | 8921 | CA | LYS | C | 263 | 48.274 | −12.755 | 2.866 | 1.00 | 100.26 | C | |
| ATOM | 8923 | CB | LYS | C | 263 | 48.863 | −11.503 | 2.191 | 1.00 | 100.47 | C | |
| ATOM | 8926 | CG | LYS | C | 263 | 50.044 | −10.758 | 2.825 | 1.00 | 100.44 | C | |
| ATOM | 8929 | CD | LYS | C | 263 | 50.305 | −9.545 | 1.911 | 1.00 | 100.63 | C | |
| ATOM | 8932 | CE | LYS | C | 263 | 51.438 | −8.650 | 2.334 | 1.00 | 100.87 | C | |
| ATOM | 8935 | NZ | LYS | C | 263 | 51.427 | −7.416 | 1.478 | 1.00 | 100.34 | N | |
| ATOM | 8939 | C | LYS | C | 263 | 47.950 | −12.444 | 4.347 | 1.00 | 99.91 | C | |
| ATOM | 8940 | O | LYS | C | 263 | 48.749 | −12.766 | 5.226 | 1.00 | 99.93 | O | |
| ATOM | 8942 | N | TYR | C | 264 | 46.804 | −11.805 | 4.599 | 1.00 | 99.27 | N | |
| ATOM | 8943 | CA | TYR | C | 264 | 46.383 | −11.395 | 5.945 | 1.00 | 98.82 | C | |
| ATOM | 8945 | CB | TYR | C | 264 | 45.985 | −9.913 | 5.942 | 1.00 | 98.51 | C | |
| ATOM | 8948 | CG | TYR | C | 264 | 46.981 | −8.964 | 5.277 | 1.00 | 98.09 | C | |
| ATOM | 8949 | CD1 | TYR | C | 264 | 48.279 | −8.790 | 5.778 | 1.00 | 97.70 | C | |
| ATOM | 8951 | CE1 | TYR | C | 264 | 49.196 | −7.886 | 5.159 | 1.00 | 97.83 | C | |
| ATOM | 8953 | CZ | TYR | C | 264 | 48.804 | −7.154 | 4.037 | 1.00 | 97.99 | C | |
| ATOM | 8954 | OH | TYR | C | 264 | 49.659 | −6.270 | 3.408 | 1.00 | 97.04 | O | |
| ATOM | 8956 | CE2 | TYR | C | 264 | 47.524 | −7.322 | 3.530 | 1.00 | 98.60 | C | |
| ATOM | 8958 | CD2 | TYR | C | 264 | 46.615 | −8.215 | 4.155 | 1.00 | 98.55 | C | |
| ATOM | 8960 | C | TYR | C | 264 | 45.220 | −12.285 | 6.406 | 1.00 | 98.17 | C | |
| ATOM | 8961 | O | TYR | C | 264 | 44.418 | −11.920 | 7.266 | 1.00 | 97.16 | O | |
| ATOM | 8963 | N | THR | C | 267 | 39.439 | −11.242 | 5.086 | 1.00 | 99.88 | N | |
| ATOM | 8964 | CA | THR | C | 267 | 38.257 | −10.991 | 4.264 | 1.00 | 99.96 | C | |
| ATOM | 8966 | CB | THR | C | 267 | 36.983 | −11.599 | 4.941 | 1.00 | 99.78 | C | |
| ATOM | 8968 | OG1 | THR | C | 267 | 37.096 | −13.029 | 4.981 | 1.00 | 99.22 | O | |
| ATOM | 8970 | CG2 | THR | C | 267 | 35.719 | −11.381 | 4.112 | 1.00 | 99.73 | C | |
| ATOM | 8974 | C | THR | C | 267 | 38.110 | −9.482 | 3.952 | 1.00 | 100.07 | C | |
| ATOM | 8975 | O | THR | C | 267 | 38.279 | −8.631 | 4.839 | 1.00 | 99.68 | O | |
| ATOM | 8977 | N | VAL | C | 268 | 37.819 | −9.182 | 2.678 | 1.00 | 100.39 | N | |
| ATOM | 8978 | CA | VAL | C | 268 | 37.606 | −7.806 | 2.172 | 1.00 | 100.41 | C | |
| ATOM | 8980 | CB | VAL | C | 268 | 38.913 | −7.181 | 1.584 | 1.00 | 100.39 | C | |
| ATOM | 8982 | CG1 VAL | C | 268 | 39.827 | −6.698 | 2.699 | 1.00 | 100.54 | C | | |
| ATOM | 8986 | CG2 | VAL | C | 268 | 39.654 | −8.154 | 0.667 | 1.00 | 100.69 | C | |
| ATOM | 8990 | C | VAL | C | 268 | 36.471 | −7.719 | 1.127 | 1.00 | 100.62 | C | |
| ATOM | 8991 | O | VAL | C | 268 | 36.095 | −8.730 | 0.514 | 1.00 | 100.73 | O | |
| ATOM | 8993 | N | ALA | C | 269 | 35.937 | −6.506 | 0.955 | 1.00 | 100.66 | N | |
| ATOM | 8994 | CA | ALA | C | 269 | 34.895 | −6.222 | −0.021 | 1.00 | 100.97 | C | |
| ATOM | 8996 | CB | ALA | C | 269 | 33.877 | −5.299 | 0.567 | 1.00 | 101.03 | C | |
| ATOM | 9000 | C | ALA | C | 269 | 35.572 | −5.600 | −1.235 | 1.00 | 101.13 | C | |
| ATOM | 9001 | O | ALA | C | 269 | 36.315 | −4.637 | −1.089 | 1.00 | 101.10 | O | |
| ATOM | 9003 | N | VAL | C | 270 | 35.324 | −6.169 | −2.420 | 1.00 | 101.70 | N | |
| ATOM | 9004 | CA | VAL | C | 270 | 36.025 | −5.817 | −3.676 | 1.00 | 102.13 | C | |
| ATOM | 9006 | CB | VAL | C | 270 | 36.998 | −6.957 | −4.133 | 1.00 | 102.14 | C | |
| ATOM | 9008 | CG1 | VAL | C | 270 | 37.547 | −6.727 | −5.542 | 1.00 | 101.98 | C | |
| ATOM | 9012 | CG2 | VAL | C | 270 | 38.151 | −7.090 | −3.151 | 1.00 | 102.21 | C | |
| ATOM | 9016 | C | VAL | C | 270 | 35.020 | −5.481 | −4.796 | 1.00 | 102.60 | C | |
| ATOM | 9017 | O | VAL | C | 270 | 34.208 | −6.337 | −5.167 | 1.00 | 102.80 | O | |
| ATOM | 9019 | N | LYS | C | 271 | 35.109 | −4.258 | −5.338 | 1.00 | 103.03 | N | |
| ATOM | 9020 | CA | LYS | C | 271 | 34.179 | −3.746 | −6.359 | 1.00 | 103.33 | C | |
| ATOM | 9022 | CB | LYS | C | 271 | 33.759 | −2.290 | −6.035 | 1.00 | 103.24 | C | |
| ATOM | 9025 | CG | LYS | C | 271 | 33.252 | −1.490 | −7.262 | 1.00 | 103.58 | C | |
| ATOM | 9028 | CD | LYS | C | 271 | 32.318 | −0.341 | −6.950 | 1.00 | 103.33 | C | |
| ATOM | 9031 | CE | LYS | C | 271 | 31.850 | 0.292 | −8.250 | 1.00 | 102.48 | C | |
| ATOM | 9034 | NZ | LYS | C | 271 | 30.685 | 1.115 | −7.974 | 1.00 | 103.09 | N | |
| ATOM | 9038 | C | LYS | C | 271 | 34.833 | −3.833 | −7.751 | 1.00 | 103.68 | C | |
| ATOM | 9039 | O | LYS | C | 271 | 35.926 | −3.315 | −7.922 | 1.00 | 103.32 | O | |
| ATOM | 9041 | N | THR | C | 272 | 34.121 | −4.421 | −8.729 | 1.00 | 104.18 | N | |
| ATOM | 9042 | CA | THR | C | 272 | 34.618 | −4.642 | −10.115 | 1.00 | 104.48 | C | |
| ATOM | 9044 | CB | THR | C | 272 | 34.731 | −6.195 | −10.452 | 1.00 | 104.62 | C | |
| ATOM | 9046 | OG1 | THR | C | 272 | 35.540 | −6.390 | −11.620 | 1.00 | 104.99 | O | |
| ATOM | 9048 | CG2 | THR | C | 272 | 33.376 | −6.856 | −10.830 | 1.00 | 104.16 | C | |
| ATOM | 9052 | C | THR | C | 272 | 33.798 | −3.895 | −11.194 | 1.00 | 104.84 | C | |
| ATOM | 9053 | O | THR | C | 272 | 32.619 | −3.570 | −10.976 | 1.00 | 104.74 | O | |
| ATOM | 9055 | N | LEU | C | 273 | 34.447 | −3.662 | −12.349 | 1.00 | 105.16 | N | |
| ATOM | 9056 | CA | LEU | C | 273 | 33.908 | −2.897 | −13.493 | 1.00 | 105.15 | C | |
| ATOM | 9058 | CB | LEU | C | 273 | 34.895 | −1.787 | −13.890 | 1.00 | 105.19 | C | |
| ATOM | 9061 | CG | LEU | C | 273 | 34.521 | −0.752 | −14.975 | 1.00 | 105.28 | C | |
| ATOM | 9063 | CD1 | LEU | C | 273 | 33.257 | 0.030 | −14.681 | 1.00 | 105.26 | C | |
| ATOM | 9067 | CD2 | LEU | C | 273 | 35.670 | 0.225 | −15.203 | 1.00 | 105.19 | C | |
| ATOM | 9071 | C | LEU | C | 273 | 33.668 | −3.836 | −14.684 | 1.00 | 105.40 | C | |
| ATOM | 9072 | O | LEU | C | 273 | 34.472 | −4.753 | −14.934 | 1.00 | 105.48 | O | |
| ATOM | 9074 | N | LYS | C | 274 | 32.592 | −3.573 | −15.438 | 1.00 | 105.39 | N | |
| ATOM | 9075 | CA | LYS | C | 274 | 32.065 | −4.521 | −16.448 | 1.00 | 105.24 | C | |
| ATOM | 9077 | CB | LYS | C | 274 | 30.558 | −4.266 | −16.698 | 1.00 | 105.27 | C | |
| ATOM | 9080 | CG | LYS | C | 274 | 29.675 | −4.302 | −15.411 | 1.00 | 105.20 | C | |
| ATOM | 9083 | CD | LYS | C | 274 | 28.191 | −4.025 | −15.698 | 1.00 | 105.02 | C | |
| ATOM | 9086 | CE | LYS | C | 274 | 27.350 | −4.012 | −14.428 | 1.00 | 104.29 | C | |
| ATOM | 9089 | NZ | LYS | C | 274 | 27.736 | −2.929 | −13.483 | 1.00 | 103.53 | N | |
| ATOM | 9093 | C | LYS | C | 274 | 32.869 | −4.464 | −17.753 | 1.00 | 105.09 | C | |
| ATOM | 9094 | O | LYS | C | 274 | 32.383 | −4.826 | −18.830 | 1.00 | 104.84 | O | |
| ATOM | 9096 | N | ASP | C | 276 | 35.241 | −3.799 | −19.931 | 1.00 | 100.89 | N | |
| ATOM | 9097 | CA | ASP | C | 276 | 35.626 | −2.705 | −20.846 | 1.00 | 100.82 | C | |
| ATOM | 9099 | CB | ASP | C | 276 | 35.925 | −3.262 | −22.265 | 1.00 | 100.90 | C | |
| ATOM | 9102 | CG | ASP | C | 276 | 34.764 | −4.076 | −22.860 | 1.00 | 100.82 | C | |
| ATOM | 9103 | OD1 | ASP | C | 276 | 34.413 | −5.145 | −22.302 | 1.00 | 100.23 | O | |
| ATOM | 9104 | OD2 | ASP | C | 276 | 34.162 | −3.730 | −23.897 | 1.00 | 100.56 | O | |
| ATOM | 9105 | C | ASP | C | 276 | 34.644 | −1.498 | −20.939 | 1.00 | 100.83 | C | |
| ATOM | 9106 | O | ASP | C | 276 | 34.994 | −0.466 | −21.530 | 1.00 | 100.85 | O | |
| ATOM | 9108 | N | THR | C | 277 | 33.464 | −1.594 | −20.316 | 1.00 | 100.64 | N | |
| ATOM | 9109 | CA | THR | C | 277 | 32.297 | −0.797 | −20.735 | 1.00 | 100.50 | C | |
| ATOM | 9111 | CB | THR | C | 277 | 30.975 | −1.542 | −20.343 | 1.00 | 100.49 | C | |
| ATOM | 9113 | OG1 | THR | C | 277 | 30.951 | −2.833 | −20.975 | 1.00 | 100.09 | O | |
| ATOM | 9115 | CG2 | THR | C | 277 | 29.706 | −0.850 | −20.894 | 1.00 | 100.30 | C | |
| ATOM | 9119 | C | THR | C | 277 | 32.292 | 0.694 | −20.285 | 1.00 | 100.56 | C | |
| ATOM | 9120 | O | THR | C | 277 | 32.822 | 1.047 | −19.219 | 1.00 | 100.64 | O | |
| ATOM | 9122 | N | MET | C | 278 | 31.705 | 1.516 | −21.174 | 1.00 | 100.38 | N | |
| ATOM | 9123 | CA | MET | C | 278 | 31.380 | 2.979 | −21.083 | 1.00 | 100.16 | C | |
| ATOM | 9125 | CB | MET | C | 278 | 29.871 | 3.166 | −21.318 | 1.00 | 100.17 | C | |
| ATOM | 9128 | CG | MET | C | 278 | 29.471 | 3.092 | −22.784 | 1.00 | 101.06 | C | |
| ATOM | 9131 | SD | MET | C | 278 | 27.691 | 3.284 | −22.990 | 1.00 | 102.76 | S | |
| ATOM | 9132 | CE | MET | C | 278 | 27.050 | 1.702 | −22.153 | 1.00 | 102.46 | C | |
| ATOM | 9136 | C | MET | C | 278 | 31.775 | 3.893 | −19.913 | 1.00 | 99.41 | C | |
| ATOM | 9137 | O | MET | C | 278 | 32.518 | 4.861 | −20.112 | 1.00 | 99.48 | O | |
| ATOM | 9139 | N | GLU | C | 279 | 31.235 | 3.626 | −18.725 | 1.00 | 98.50 | N | |
| ATOM | 9140 | CA | GLU | C | 279 | 31.389 | 4.522 | −17.553 | 1.00 | 97.55 | C | |
| ATOM | 9142 | CB | GLU | C | 279 | 30.063 | 4.561 | −16.754 | 1.00 | 97.90 | C | |
| ATOM | 9145 | CG | GLU | C | 279 | 28.897 | 5.263 | −17.484 | 1.00 | 98.44 | C | |
| ATOM | 9148 | CD | GLU | C | 279 | 27.518 | 4.724 | −17.074 | 1.00 | 99.13 | C | |
| ATOM | 9149 | OE1 | GLU | C 279 | 27.188 | 3.580 | −17.489 | 1.00 | 101.98 | O | | |
| ATOM | 9150 | OE2 | GLU | C 279 | 26.762 | 5.422 | −16.339 | 1.00 | 99.66 | O | | |
| ATOM | 9151 | C | GLU | C | 279 | 32.632 | 4.140 | −16.692 | 1.00 | 96.00 | C | |
| ATOM | 9152 | O | GLU | C | 279 | 32.555 | 4.014 | −15.470 | 1.00 | 95.28 | O | |
| ATOM | 9154 | N | VAL | C | 280 | 33.775 | 4.001 | −17.374 | 1.00 | 94.45 | N | |
| ATOM | 9155 | CA | VAL | C | 280 | 35.097 | 3.737 | −16.777 | 1.00 | 93.59 | C | |
| ATOM | 9157 | CB | VAL | C | 280 | 36.198 | 3.456 | −17.881 | 1.00 | 93.13 | C | |
| ATOM | 9159 | CG1 | VAL | C | 280 | 37.595 | 3.243 | −17.266 | 1.00 | 92.50 | C | |
| ATOM | 9163 | CG2 | VAL | C | 280 | 35.806 | 2.256 | −18.785 | 1.00 | 93.44 | C | |
| ATOM | 9167 | C | VAL | C | 280 | 35.546 | 4.933 | −15.940 | 1.00 | 91.75 | C | |
| ATOM | 9168 | O | VAL | C | 280 | 36.192 | 4.775 | −14.907 | 1.00 | 91.50 | O | |
| ATOM | 9170 | N | GLU | C | 281 | 35.224 | 6.133 | −16.405 | 1.00 | 90.30 | N | |
| ATOM | 9171 | CA | GLU | C | 281 | 35.582 | 7.332 | −15.686 | 1.00 | 88.98 | C | |
| ATOM | 9173 | CB | GLU | C | 281 | 35.353 | 8.560 | −16.572 | 1.00 | 88.85 | C | |
| ATOM | 9176 | CG | GLU | C | 281 | 35.815 | 9.872 | −15.959 | 1.00 | 90.21 | C | |
| ATOM | 9179 | CD | GLU | C | 281 | 37.148 | 9.766 | −15.214 | 1.00 | 91.90 | C | |
| ATOM | 9180 | OE1 | GLU | C | 281 | 38.147 | 9.338 | −15.874 | 1.00 | 92.80 | O | |
| ATOM | 9181 | OE2 | GLU | C | 281 | 37.179 | 10.100 | −13.988 | 1.00 | 90.02 | O | |
| ATOM | 9182 | C | GLU | C | 281 | 34.867 | 7.450 | −14.324 | 1.00 | 87.72 | C | |
| ATOM | 9183 | O | GLU | C | 281 | 35.468 | 7.900 | −13.350 | 1.00 | 87.59 | O | |
| ATOM | 9185 | N | GLU | C | 282 | 33.613 | 7.011 | −14.253 | 1.00 | 86.25 | N | |
| ATOM | 9186 | CA | GLU | C | 282 | 32.860 | 7.029 | −13.003 | 1.00 | 85.24 | C | |
| ATOM | 9188 | CB | GLU | C | 282 | 31.431 | 6.515 | −13.180 | 1.00 | 85.21 | C | |
| ATOM | 9191 | CG | GLU | C | 282 | 30.548 | 7.259 | −14.171 | 1.00 | 85.95 | C | |
| ATOM | 9194 | CD | GLU | C | 282 | 30.460 | 8.745 | −13.908 | 1.00 | 87.00 | C | |
| ATOM | 9195 | OE1 | GLU | C | 282 | 30.426 | 9.147 | −12.725 | 1.00 | 87.53 | O | |
| ATOM | 9196 | OE2 | GLU | C | 282 | 30.421 | 9.514 | −14.895 | 1.00 | 88.12 | O | |
| ATOM | 9197 | C | GLU | C | 282 | 33.532 | 6.151 | −11.975 | 1.00 | 84.64 | C | |
| ATOM | 9198 | O | GLU | C | 282 | 33.686 | 6.547 | −10.825 | 1.00 | 85.03 | O | |
| ATOM | 9200 | N | PHE | C | 283 | 33.925 | 4.957 | −12.401 | 1.00 | 83.65 | N | |
| ATOM | 9201 | CA | PHE | C | 283 | 34.620 | 3.995 | −11.544 | 1.00 | 82.99 | C | |
| ATOM | 9203 | CB | PHE | C | 283 | 34.991 | 2.782 | −12.374 | 1.00 | 83.26 | C | |
| ATOM | 9206 | CG | PHE | C | 283 | 35.366 | 1.600 | −11.578 | 1.00 | 83.65 | C | |
| ATOM | 9207 | CD1 | PHE | C | 283 | 34.394 | 0.682 | −11.186 | 1.00 | 84.25 | C | |
| ATOM | 9209 | CE1 | PHE | C | 283 | 34.749 | −0.459 | −10.470 | 1.00 | 85.52 | C | |
| ATOM | 9211 | CZ | PHE | C | 283 | 36.105 | −0.685 | −10.142 | 1.00 | 85.31 | C | |
| ATOM | 9213 | CE2 | PHE | C | 283 | 37.079 | 0.224 | −10.551 | 1.00 | 84.69 | C | |
| ATOM | 9215 | CD2 | PHE | C | 283 | 36.704 | 1.359 | −11.263 | 1.00 | 84.21 | C | |
| ATOM | 9217 | C | PHE | C | 283 | 35.872 | 4.613 | −10.934 | 1.00 | 82.29 | C | |
| ATOM | 9218 | O | PHE | C | 283 | 36.105 | 4.566 | −9.712 | 1.00 | 81.85 | O | |
| ATOM | 9220 | N | LEU | C | 284 | 36.653 | 5.225 | −11.811 | 1.00 | 81.41 | N | |
| ATOM | 9221 | CA | LEU | C | 284 | 37.845 | 5.923 | −11.401 | 1.00 | 80.93 | C | |
| ATOM | 9223 | CB | LEU | C | 284 | 38.697 | 6.302 | −12.616 | 1.00 | 80.65 | C | |
| ATOM | 9226 | CG | LEU | C | 284 | 39.420 | 5.138 | −13.302 | 1.00 | 78.38 | C | |
| ATOM | 9228 | CD1 | LEU | C | 284 | 39.918 | 5.580 | −14.627 | 1.00 | 76.78 | C | |
| ATOM | 9232 | CD2 | LEU | C | 284 | 40.571 | 4.623 | −12.459 | 1.00 | 77.49 | C | |
| ATOM | 9236 | C | LEU | C | 284 | 37.584 | 7.139 | −10.495 | 1.00 | 81.17 | C | |
| ATOM | 9237 | O | LEU | C | 284 | 38.293 | 7.269 | −9.507 | 1.00 | 81.27 | O | |
| ATOM | 9239 | N | LYS | C | 285 | 36.596 | 8.007 | −10.793 | 1.00 | 81.33 | N | |
| ATOM | 9240 | CA | LYS | C | 285 | 36.178 | 9.061 | −9.812 | 1.00 | 81.30 | C | |
| ATOM | 9242 | CB | LYS | C | 285 | 35.007 | 9.937 | −10.302 | 1.00 | 81.06 | C | |
| ATOM | 9245 | CG | LYS | C | 285 | 35.422 | 11.235 | −11.006 | 1.00 | 81.70 | C | |
| ATOM | 9248 | CD | LYS | C | 285 | 34.722 | 12.505 | −10.454 | 1.00 | 80.22 | C | |
| ATOM | 9251 | CE | LYS | C | 285 | 35.368 | 13.793 | −11.032 | 1.00 | 79.42 | C | |
| ATOM | 9254 | NZ | LYS | C | 285 | 35.194 | 14.966 | −10.156 | 1.00 | 77.51 | N | |
| ATOM | 9258 | C | LYS | C | 285 | 35.840 | 8.493 | −8.406 | 1.00 | 81.57 | C | |
| ATOM | 9259 | O | LYS | C | 285 | 36.190 | 9.109 | −7.401 | 1.00 | 81.77 | O | |
| ATOM | 9261 | N | GLU | C | 286 | 35.224 | 7.311 | −8.352 | 1.00 | 81.38 | N | |
| ATOM | 9262 | CA | GLU | C | 286 | 34.880 | 6.684 | −7.091 | 1.00 | 81.53 | C | |
| ATOM | 9264 | CB | GLU | C | 286 | 33.965 | 5.480 | −7.316 | 1.00 | 81.68 | C | |
| ATOM | 9267 | CG | GLU | C | 286 | 33.207 | 5.051 | −6.081 | 1.00 | 81.50 | C | |
| ATOM | 9270 | CD | GLU | C | 286 | 32.427 | 3.760 | −6.256 | 1.00 | 82.69 | C | |
| ATOM | 9271 | OE1 | GLU | C | 286 | 32.273 | 3.269 | −7.400 | 1.00 | 81.77 | O | |
| ATOM | 9272 | OE2 | GLU | C | 286 | 31.944 | 3.233 | −5.217 | 1.00 | 87.14 | O | |
| ATOM | 9273 | C | GLU | C | 286 | 36.129 | 6.271 | −6.290 | 1.00 | 81.65 | C | |
| ATOM | 9274 | O | GLU | C | 286 | 36.240 | 6.612 | −5.106 | 1.00 | 81.94 | O | |
| ATOM | 9276 | N | ALA | C | 287 | 37.056 | 5.548 | −6.930 | 1.00 | 81.64 | N | |
| ATOM | 9277 | CA | ALA | C | 287 | 38.339 | 5.171 | −6.299 | 1.00 | 81.16 | C | |
| ATOM | 9279 | CB | ALA | C | 287 | 39.245 | 4.470 | −7.292 | 1.00 | 80.91 | C | |
| ATOM | 9283 | C | ALA | C | 287 | 39.035 | 6.407 | −5.730 | 1.00 | 81.03 | C | |
| ATOM | 9284 | O | ALA | C | 287 | 39.408 | 6.426 | −4.563 | 1.00 | 81.57 | O | |
| ATOM | 9286 | N | ALA | C | 288 | 39.122 | 7.455 | −6.546 | 1.00 | 80.81 | N | |
| ATOM | 9287 | CA | ALA | C | 288 | 39.813 | 8.696 | −6.206 | 1.00 | 80.72 | C | |
| ATOM | 9289 | CB | ALA | C | 288 | 39.877 | 9.580 | −7.419 | 1.00 | 80.90 | C | |
| ATOM | 9293 | C | ALA | C | 288 | 39.209 | 9.475 | −5.043 | 1.00 | 80.79 | C | |
| ATOM | 9294 | O | ALA | C | 288 | 39.939 | 10.133 | −4.317 | 1.00 | 80.49 | O | |
| ATOM | 9296 | N | VAL | C | 289 | 37.886 | 9.436 | −4.887 | 1.00 | 81.36 | N | |
| ATOM | 9297 | CA | VAL | C | 289 | 37.220 | 9.983 | −3.683 | 1.00 | 81.52 | C | |
| ATOM | 9299 | CB | VAL | C | 289 | 35.658 | 10.008 | −3.795 | 1.00 | 81.36 | C | |
| ATOM | 9301 | CG1 | VAL | C | 289 | 34.962 | 10.338 | −2.406 | 1.00 | 81.72 | C | |
| ATOM | 9305 | CG2 | VAL | C | 289 | 35.220 | 11.026 | −4.844 | 1.00 | 81.07 | C | |
| ATOM | 9309 | C | VAL | C | 289 | 37.652 | 9.191 | −2.445 | 1.00 | 81.83 | C | |
| ATOM | 9310 | O | VAL | C | 289 | 38.088 | 9.792 | −1.461 | 1.00 | 81.84 | O | |
| ATOM | 9312 | N | MET | C | 290 | 37.542 | 7.863 | −2.507 | 1.00 | 82.18 | N | |
| ATOM | 9313 | CA | MET | C | 290 | 37.907 | 6.987 | −1.378 | 1.00 | 82.68 | C | |
| ATOM | 9315 | CB | MET | C | 290 | 37.620 | 5.538 | −1.731 | 1.00 | 82.45 | C | |
| ATOM | 9318 | CG | MET | C | 290 | 36.156 | 5.229 | −1.871 | 1.00 | 82.92 | C | |
| ATOM | 9321 | SD | MET | C | 290 | 35.864 | 3.459 | −1.850 | 1.00 | 82.66 | S | |
| ATOM | 9322 | CE | MET | C | 290 | 34.174 | 3.303 | −2.618 | 1.00 | 83.15 | C | |
| ATOM | 9326 | C | MET | C | 290 | 39.366 | 7.121 | −0.849 | 1.00 | 83.48 | C | |
| ATOM | 9327 | O | MET | C | 290 | 39.639 | 6.856 | 0.342 | 1.00 | 84.08 | O | |
| ATOM | 9329 | N | LYS | C | 291 | 40.278 | 7.544 | −1.729 | 1.00 | 83.75 | N | |
| ATOM | 9330 | CA | LYS | C | 291 | 41.650 | 7.895 | −1.357 | 1.00 | 83.84 | C | |
| ATOM | 9332 | CB | LYS | C | 291 | 42.520 | 8.030 | −2.632 | 1.00 | 83.93 | C | |
| ATOM | 9335 | CG | LYS | C | 291 | 42.704 | 6.740 | −3.451 | 1.00 | 83.65 | C | |
| ATOM | 9338 | CD | LYS | C | 291 | 43.407 | 7.008 | −4.791 | 1.00 | 83.63 | C | |
| ATOM | 9341 | CE | LYS | C | 291 | 43.795 | 5.708 | −5.536 | 1.00 | 83.90 | C | |
| ATOM | 9344 | NZ | LYS | C | 291 | 44.183 | 5.879 | −7.001 | 1.00 | 82.56 | N | |
| ATOM | 9348 | C | LYS | C | 291 | 41.815 | 9.178 | −0.494 | 1.00 | 84.12 | C | |
| ATOM | 9349 | O | LYS | C | 291 | 42.919 | 9.433 | −0.030 | 1.00 | 84.75 | O | |
| ATOM | 9351 | N | GLU | C | 292 | 40.771 | 9.988 | −0.294 | 1.00 | 84.59 | N | |
| ATOM | 9352 | CA | GLU | C | 292 | 40.839 | 11.265 | 0.502 | 1.00 | 85.16 | C | |
| ATOM | 9354 | CB | GLU | C | 292 | 40.205 | 12.440 | −0.297 | 1.00 | 85.84 | C | |
| ATOM | 9357 | CG | GLU | C | 292 | 40.640 | 12.654 | −1.760 | 1.00 | 87.07 | C | |
| ATOM | 9360 | CD | GLU | C | 292 | 42.015 | 13.272 | −1.934 | 1.00 | 88.47 | C | |
| ATOM | 9361 | OE1 | GLU | C | 292 | 42.611 | 13.696 | −0.926 | 1.00 | 89.14 | O | |
| ATOM | 9362 | OE2 | GLU | C | 292 | 42.499 | 13.331 | −3.099 | 1.00 | 90.13 | O | |
| ATOM | 9363 | C | GLU | C | 292 | 40.109 | 11.224 | 1.892 | 1.00 | 85.46 | C | |
| ATOM | 9364 | O | GLU | C | 292 | 40.016 | 12.271 | 2.588 | 1.00 | 85.32 | O | |
| ATOM | 9366 | N | ILE | C | 293 | 39.579 | 10.051 | 2.276 | 1.00 | 85.38 | N | |
| ATOM | 9367 | CA | ILE | C | 293 | 38.576 | 9.943 | 3.345 | 1.00 | 85.03 | C | |
| ATOM | 9369 | CB | ILE | C | 293 | 37.117 | 9.688 | 2.767 | 1.00 | 84.91 | C | |
| ATOM | 9371 | CG1 | ILE | C | 293 | 36.957 | 8.296 | 2.117 | 1.00 | 84.84 | C | |
| ATOM | 9374 | CD1 | ILE | C | 293 | 35.563 | 8.007 | 1.542 | 1.00 | 84.81 | C | |
| ATOM | 9378 | CG2 | ILE | C | 293 | 36.728 | 10.815 | 1.796 | 1.00 | 84.36 | C | |
| ATOM | 9382 | C | ILE | C | 293 | 39.011 | 8.869 | 4.326 | 1.00 | 85.06 | C | |
| ATOM | 9383 | O | ILE | C | 293 | 39.334 | 7.741 | 3.908 | 1.00 | 85.46 | O | |
| ATOM | 9385 | N | LYS | C | 294 | 39.035 | 9.239 | 5.613 | 1.00 | 84.87 | N | |
| ATOM | 9386 | CA | LYS | C | 294 | 39.474 | 8.368 | 6.708 | 1.00 | 84.53 | C | |
| ATOM | 9388 | CB | LYS | C | 294 | 40.950 | 8.663 | 7.056 | 1.00 | 84.40 | C | |
| ATOM | 9391 | CG | LYS | C | 294 | 41.664 | 7.516 | 7.716 | 1.00 | 84.15 | C | |
| ATOM | 9394 | CD | LYS | C | 294 | 43.006 | 7.922 | 8.303 | 1.00 | 84.31 | C | |
| ATOM | 9397 | CE | LYS | C | 294 | 43.656 | 6.763 | 9.084 | 1.00 | 83.87 | C | |
| ATOM | 9400 | NZ | LYS | C | 294 | 45.123 | 6.933 | 9.192 | 1.00 | 83.28 | N | |
| ATOM | 9404 | C | LYS | C | 294 | 38.552 | 8.636 | 7.893 | 1.00 | 84.42 | C | |
| ATOM | 9405 | O | LYS | C | 294 | 38.587 | 9.734 | 8.459 | 1.00 | 84.14 | O | |
| ATOM | 9407 | N | HIS | C | 295 | 37.696 | 7.659 | 8.217 | 1.00 | 84.45 | N | |
| ATOM | 9408 | CA | HIS | C | 295 | 36.768 | 7.741 | 9.370 | 1.00 | 84.30 | C | |
| ATOM | 9410 | CB | HIS | C | 295 | 35.633 | 8.732 | 9.066 | 1.00 | 84.68 | C | |
| ATOM | 9413 | CG | HIS | C | 295 | 34.858 | 9.145 | 10.270 | 1.00 | 84.83 | C | |
| ATOM | 9414 | ND1 | HIS | C | 295 | 33.906 | 8.334 | 10.840 | 1.00 | 84.57 | N | |
| ATOM | 9416 | CE1 | HIS | C | 295 | 33.390 | 8.937 | 11.890 | 1.00 | 85.47 | C | |
| ATOM | 9418 | NE2 | HIS | C | 295 | 33.974 | 10.114 | 12.026 | 1.00 | 85.78 | N | |
| ATOM | 9420 | CD2 | HIS | C | 295 | 34.899 | 10.268 | 11.023 | 1.00 | 85.29 | C | |
| ATOM | 9422 | C | HIS | C | 295 | 36.174 | 6.343 | 9.689 | 1.00 | 84.03 | C | |
| ATOM | 9423 | O | HIS | C | 295 | 35.891 | 5.585 | 8.756 | 1.00 | 84.23 | O | |
| ATOM | 9425 | N | PRO | C | 296 | 35.994 | 5.975 | 10.961 | 1.00 | 83.71 | N | |
| ATOM | 9426 | CA | PRO | C | 296 | 35.501 | 4.621 | 11.272 | 1.00 | 83.67 | C | |
| ATOM | 9428 | CB | PRO | C | 296 | 35.956 | 4.396 | 12.732 | 1.00 | 83.24 | C | |
| ATOM | 9431 | CG | PRO | C | 296 | 36.028 | 5.738 | 13.322 | 1.00 | 83.35 | C | |
| ATOM | 9434 | CD | PRO | C | 296 | 36.326 | 6.708 | 12.200 | 1.00 | 83.94 | C | |
| ATOM | 9437 | C | PRO | C | 296 | 33.995 | 4.371 | 11.093 | 1.00 | 83.72 | C | |
| ATOM | 9438 | O | PRO | C | 296 | 33.537 | 3.290 | 11.468 | 1.00 | 84.53 | O | |
| ATOM | 9439 | N | ASN | C | 297 | 33.234 | 5.335 | 10.568 | 1.00 | 83.07 | N | |
| ATOM | 9440 | CA | ASN | C | 297 | 31.884 | 5.081 | 10.031 | 1.00 | 82.79 | C | |
| ATOM | 9442 | CB | ASN | C | 297 | 30.834 | 5.758 | 10.874 | 1.00 | 82.41 | C | |
| ATOM | 9445 | CG | ASN | C | 297 | 30.949 | 5.387 | 12.295 | 1.00 | 80.98 | C | |
| ATOM | 9446 | OD1 | ASN | C | 297 | 31.191 | 6.246 | 13.133 | 1.00 | 80.80 | O | |
| ATOM | 9447 | ND2 | ASN | C | 297 | 30.821 | 4.099 | 12.591 | 1.00 | 77.48 | N | |
| ATOM | 9450 | C | ASN | C | 297 | 31.753 | 5.465 | 8.560 | 1.00 | 82.84 | C | |
| ATOM | 9451 | O | ASN | C | 297 | 30.671 | 5.855 | 8.104 | 1.00 | 82.20 | O | |
| ATOM | 9453 | N | LEU | C | 298 | 32.888 | 5.356 | 7.848 | 1.00 | 83.10 | N | |
| ATOM | 9454 | CA | LEU | C | 298 | 32.971 | 5.276 | 6.389 | 1.00 | 83.04 | C | |
| ATOM | 9456 | CB | LEU | C | 298 | 33.848 | 6.399 | 5.847 | 1.00 | 82.80 | C | |
| ATOM | 9459 | CG | LEU | C | 298 | 33.422 | 7.831 | 6.152 | 1.00 | 83.15 | C | |
| ATOM | 9461 | CD1 | LEU | C | 298 | 34.525 | 8.812 | 5.783 | 1.00 | 83.39 | C | |
| ATOM | 9465 | CD2 | LEU | C | 298 | 32.156 | 8.195 | 5.409 | 1.00 | 83.91 | C | |
| ATOM | 9469 | C | LEU | C | 298 | 33.642 | 3.956 | 6.096 | 1.00 | 83.10 | C | |
| ATOM | 9470 | O | LEU | C | 298 | 34.708 | 3.687 | 6.661 | 1.00 | 83.47 | O | |
| ATOM | 9472 | N | VAL | C | 299 | 33.033 | 3.117 | 5.265 | 1.00 | 83.23 | N | |
| ATOM | 9473 | CA | VAL | C | 299 | 33.712 | 1.899 | 4.810 | 1.00 | 83.99 | C | |
| ATOM | 9475 | CB | VAL | C | 299 | 32.786 | 0.994 | 3.935 | 1.00 | 83.47 | C | |
| ATOM | 9477 | CG1 | VAL | C | 299 | 32.363 | 1.660 | 2.668 | 1.00 | 84.36 | C | |
| ATOM | 9481 | CG2 | VAL | C | 299 | 33.458 | −0.313 | 3.603 | 1.00 | 83.89 | C | |
| ATOM | 9485 | C | VAL | C | 299 | 35.086 | 2.293 | 4.157 | 1.00 | 84.34 | C | |
| ATOM | 9486 | O | VAL | C | 299 | 35.114 | 3.102 | 3.236 | 1.00 | 84.19 | O | |
| ATOM | 9488 | N | GLN | C | 300 | 36.202 | 1.793 | 4.724 | 1.00 | 85.15 | N | |
| ATOM | 9489 | CA | GLN | C | 300 | 37.594 | 2.247 | 4.393 | 1.00 | 85.42 | C | |
| ATOM | 9491 | CB | GLN | C | 300 | 38.551 | 2.012 | 5.561 | 1.00 | 85.44 | C | |
| ATOM | 9494 | CG | GLN | C | 300 | 40.012 | 2.492 | 5.361 | 1.00 | 85.30 | C | |
| ATOM | 9497 | CD | GLN | C | 300 | 40.191 | 3.984 | 5.534 | 1.00 | 86.17 | C | |
| ATOM | 9498 | OE1 | GLN | C | 300 | 39.772 | 4.773 | 4.689 | 1.00 | 85.60 | O | |
| ATOM | 9499 | NE2 | GLN | C | 300 | 40.828 | 4.377 | 6.640 | 1.00 | 87.22 | N | |
| ATOM | 9502 | C | GLN | C | 300 | 38.222 | 1.575 | 3.169 | 1.00 | 86.32 | C | |
| ATOM | 9503 | O | GLN | C | 300 | 38.295 | 0.352 | 3.109 | 1.00 | 86.15 | O | |
| ATOM | 9505 | N | LEU | C | 301 | 38.739 | 2.403 | 2.255 | 1.00 | 87.35 | N | |
| ATOM | 9506 | CA | LEU | C | 301 | 39.483 | 1.955 | 1.084 | 1.00 | 87.93 | C | |
| ATOM | 9508 | CB | LEU | C | 301 | 39.600 | 3.079 | 0.048 | 1.00 | 87.94 | C | |
| ATOM | 9511 | CG | LEU | C | 301 | 40.529 | 2.915 | −1.179 | 1.00 | 87.74 | C | |
| ATOM | 9513 | CD1 | LEU | C | 301 | 39.862 | 2.041 | −2.211 | 1.00 | 87.12 | C | |
| ATOM | 9517 | CD2 | LEU | C | 301 | 40.999 | 4.272 | −1.781 | 1.00 | 87.33 | C | |
| ATOM | 9521 | C | LEU | C | 301 | 40.863 | 1.533 | 1.554 | 1.00 | 88.89 | C | |
| ATOM | 9522 | O | LEU | C | 301 | 41.549 | 2.317 | 2.223 | 1.00 | 88.74 | O | |
| ATOM | 9524 | N | LEU | C | 302 | 41.248 | 0.304 | 1.188 | 1.00 | 89.99 | N | |
| ATOM | 9525 | CA | LEU | C | 302 | 42.517 | −0.319 | 1.596 | 1.00 | 91.16 | C | |
| ATOM | 9527 | CB | LEU | C | 302 | 42.293 | −1.774 | 2.074 | 1.00 | 91.12 | C | |
| ATOM | 9530 | CG | LEU | C | 302 | 41.389 | −2.061 | 3.288 | 1.00 | 91.10 | C | |
| ATOM | 9532 | CD1 | LEU | C | 302 | 41.454 | −3.549 | 3.654 | 1.00 | 91.13 | C | |
| ATOM | 9536 | CD2 | LEU | C | 302 | 41.752 | −1.209 | 4.495 | 1.00 | 90.37 | C | |
| ATOM | 9540 | C | LEU | C | 302 | 43.513 | −0.301 | 0.426 | 1.00 | 91.99 | C | |
| ATOM | 9541 | O | LEU | C | 302 | 44.646 | 0.156 | 0.563 | 1.00 | 92.67 | O | |
| ATOM | 9543 | N | GLY | C | 303 | 43.085 | −0.834 | −0.711 | 1.00 | 92.59 | N | |
| ATOM | 9544 | CA | GLY | C | 303 | 43.860 | −0.796 | −1.943 | 1.00 | 92.82 | C | |
| ATOM | 9547 | C | GLY | C | 303 | 42.980 | −0.508 | −3.134 | 1.00 | 93.16 | C | |
| ATOM | 9548 | O | GLY | C | 303 | 41.748 | −0.367 | −3.005 | 1.00 | 93.17 | O | |
| ATOM | 9550 | N | VAL | C | 304 | 43.636 | −0.394 | −4.290 | 1.00 | 93.28 | N | |
| ATOM | 9551 | CA | VAL | C | 304 | 42.973 | −0.369 | −5.604 | 1.00 | 93.02 | C | |
| ATOM | 9553 | CB | VAL | C | 304 | 42.904 | 1.096 | −6.214 | 1.00 | 93.03 | C | |
| ATOM | 9555 | CG1 | VAL | C | 304 | 42.186 | 2.059 | −5.269 | 1.00 | 93.17 | C | |
| ATOM | 9559 | CG2 | VAL | C | 304 | 44.291 | 1.645 | −6.591 | 1.00 | 92.49 | C | |
| ATOM | 9563 | C | VAL | C | 304 | 43.736 | −1.303 | −6.534 | 1.00 | 92.96 | C | |
| ATOM | 9564 | O | VAL | C | 304 | 44.642 | −2.044 | −6.106 | 1.00 | 93.24 | O | |
| ATOM | 9566 | N | CYS | C | 305 | 43.328 | −1.289 | −7.796 | 1.00 | 92.69 | N | |
| ATOM | 9567 | CA | CYS | C | 305 | 44.228 | −1.603 | −8.911 | 1.00 | 92.47 | C | |
| ATOM | 9569 | CB | CYS | C | 305 | 44.451 | −3.103 | −9.056 | 1.00 | 92.39 | C | |
| ATOM | 9572 | SC | CYS | C | 305 | 43.066 | −4.049 | −8.444 | 1.00 | 90.89 | S | |
| ATOM | 9574 | C | CYS | C | 305 | 43.662 | −0.947 | −10.184 | 1.00 | 92.60 | C | |
| ATOM | 9575 | O | CYS | C | 305 | 42.544 | −1.268 | −10.615 | 1.00 | 92.41 | O | |
| ATOM | 9577 | N | THR | C | 306 | 44.416 | 0.021 | −10.712 | 1.00 | 92.67 | N | |
| ATOM | 9578 | CA | THR | C | 306 | 44.011 | 0.850 | −11.848 | 1.00 | 92.76 | C | |
| ATOM | 9580 | CB | THR | C | 306 | 43.475 | 2.261 | −11.355 | 1.00 | 92.56 | C | |
| ATOM | 9582 | OG1 | THR | C | 306 | 44.459 | 2.938 | −10.573 | 1.00 | 93.21 | O | |
| ATOM | 9584 | CG2 | THR | C | 306 | 42.300 | 2.139 | −10.374 | 1.00 | 91.24 | C | |
| ATOM | 9588 | C | THR | C | 306 | 45.204 | 0.913 | −12.849 | 1.00 | 92.99 | C | |
| ATOM | 9589 | O | THR | C | 306 | 45.743 | 1.988 | −13.163 | 1.00 | 92.97 | O | |
| ATOM | 9591 | N | ARG | C | 307 | 45.604 | −0.286 | −13.304 | 1.00 | 93.31 | N | |
| ATOM | 9592 | CA | ARG | C | 307 | 46.707 | −0.517 | −14.258 | 1.00 | 93.28 | C | |
| ATOM | 9594 | CB | ARG | C | 307 | 47.810 | −1.358 | −13.609 | 1.00 | 93.84 | C | |
| ATOM | 9597 | CG | ARG | C | 307 | 48.458 | −0.723 | −12.382 | 1.00 | 95.22 | C | |
| ATOM | 9600 | CD | ARG | C | 307 | 49.180 | 0.592 | −12.670 | 1.00 | 97.34 | C | |
| ATOM | 9603 | NE | ARG | C | 307 | 49.671 | 1.286 | −11.474 | 1.00 | 97.61 | N | |
| ATOM | 9605 | CZ | ARG | C | 307 | 50.232 | 2.509 | −11.476 | 1.00 | 99.72 | C | |
| ATOM | 9606 | NH1 | ARG | C | 307 | 50.403 | 3.196 | −12.617 | 1.00 | 101.42 | N | |
| ATOM | 9609 | NH2 | ARG | C | 307 | 50.644 | 3.062 | −10.332 | 1.00 | 99.75 | N | |
| ATOM | 9612 | C | ARG | C | 307 | 46.217 | −1.222 | −15.525 | 1.00 | 93.16 | C | |
| ATOM | 9613 | O | ARG | C | 307 | 46.427 | −0.710 | −16.625 | 1.00 | 93.34 | O | |
| ATOM | 9615 | N | GLU | C | 308 | 45.581 | −2.388 | −15.367 | 1.00 | 92.61 | N | |
| ATOM | 9616 | CA | GLU | C | 308 | 44.915 | −3.114 | −16.465 | 1.00 | 92.40 | C | |
| ATOM | 9618 | CB | GLU | C | 308 | 45.757 | −4.334 | −16.867 | 1.00 | 92.53 | C | |
| ATOM | 9621 | CG | GLU | C | 308 | 47.184 | −4.034 | −17.373 | 1.00 | 92.41 | C | |
| ATOM | 9624 | CD | GLU | C | 308 | 48.320 | −4.730 | −16.597 | 1.00 | 91.68 | C | |
| ATOM | 9625 | OE1 | GLU | C | 308 | 48.223 | −4.918 | −15.347 | 1.00 | 90.95 | O | |
| ATOM | 9626 | OE2 | GLU | C | 308 | 49.342 | −5.052 | −17.245 | 1.00 | 88.64 | O | |
| ATOM | 9627 | C | GLU | C | 308 | 43.511 | −3.592 | −16.022 | 1.00 | 92.15 | C | |
| ATOM | 9628 | O | GLU | C | 308 | 43.261 | −3.674 | −14.823 | 1.00 | 92.01 | O | |
| ATOM | 9630 | N | PRO | C | 309 | 42.591 | −3.887 | −16.954 | 1.00 | 92.14 | N | |
| ATOM | 9631 | CA | PRO | C | 309 | 41.321 | −4.592 | −16.630 | 1.00 | 92.09 | C | |
| ATOM | 9633 | CE | PRO | C | 309 | 40.690 | −4.826 | −18.008 | 1.00 | 91.93 | C | |
| ATOM | 9636 | CG | PRO | C | 309 | 41.192 | −3.701 | −18.823 | 1.00 | 92.00 | C | |
| ATOM | 9639 | CD | PRO | C | 309 | 42.616 | −3.493 | −18.376 | 1.00 | 92.16 | C | |
| ATOM | 9642 | C | PRO | C | 309 | 41.458 | −5.945 | −15.912 | 1.00 | 92.14 | C | |
| ATOM | 9643 | O | PRO | C | 309 | 42.584 | −6.437 | −15.765 | 1.00 | 92.29 | O | |
| ATOM | 9644 | N | PRO | C | 310 | 40.332 | −6.545 | −15.489 | 1.00 | 92.18 | N | |
| ATOM | 9645 | CA | PRO | C | 310 | 39.238 | −5.811 | −14.839 | 1.00 | 92.25 | C | |
| ATOM | 9647 | CE | PRO | C | 310 | 38.274 | −6.926 | −14.412 | 1.00 | 92.12 | C | |
| ATOM | 9650 | CG | PRO | C | 310 | 39.140 | −8.108 | −14.185 | 1.00 | 91.93 | C | |
| ATOM | 9653 | CD | PRO | C | 310 | 40.248 | −7.989 | −15.183 | 1.00 | 92.22 | C | |
| ATOM | 9656 | C | PRO | C | 310 | 39.724 | −5.018 | −13.621 | 1.00 | 92.32 | C | |
| ATOM | 9657 | O | PRO | C | 310 | 40.419 | −5.584 | −12.772 | 1.00 | 92.31 | O | |
| ATOM | 9658 | N | PHE | C | 311 | 39.375 | −3.729 | −13.555 | 1.00 | 92.21 | N | |
| ATOM | 9659 | CA | PHE | C | 311 | 39.746 | −2.882 | −12.413 | 1.00 | 92.09 | C | |
| ATOM | 9661 | CB | PHE | C | 311 | 39.547 | −1.388 | −12.700 | 1.00 | 92.42 | C | |
| ATOM | 9664 | CG | PHE | C | 311 | 40.360 | −0.869 | −13.864 | 1.00 | 93.34 | C | |
| ATOM | 9665 | CD1 | PHE | C | 311 | 41.756 | −1.048 | −13.905 | 1.00 | 93.57 | C | |
| ATOM | 9667 | CE1 | PHE | C | 311 | 42.533 | −0.548 | −14.976 | 1.00 | 92.67 | C | |
| ATOM | 9669 | CZ | PHE | C | 311 | 41.917 | 0.122 | −16.023 | 1.00 | 92.85 | C | |
| ATOM | 9671 | CE2 | PHE | C | 311 | 40.512 | 0.312 | −16.004 | 1.00 | 93.36 | C | |
| ATOM | 9673 | CD2 | PHE | C | 311 | 39.742 | −0.182 | −14.923 | 1.00 | 93.83 | C | |
| ATOM | 9675 | C | PHE | C | 311 | 38.903 | −3.273 | −11.202 | 1.00 | 91.71 | C | |
| ATOM | 9676 | O | PHE | C | 311 | 37.706 | −3.574 | −11.336 | 1.00 | 91.53 | O | |
| ATOM | 9678 | N | TYR | C | 312 | 39.554 | −3.288 | −10.038 | 1.00 | 91.20 | N | |
| ATOM | 9679 | CA | TYR | C | 312 | 38.928 | −3.593 | −8.769 | 1.00 | 90.49 | C | |
| ATOM | 9681 | CE | TYR | C | 312 | 39.545 | −4.847 | −8.144 | 1.00 | 90.91 | C | |
| ATOM | 9684 | CG | TYR | C | 312 | 39.337 | −6.184 | −8.834 | 1.00 | 90.84 | C | |
| ATOM | 9685 | CD1 | TYR | C | 312 | 38.110 | −6.518 | −9.458 | 1.00 | 91.22 | C | |
| ATOM | 9687 | CE1 | TYR | C | 312 | 37.926 | −7.792 | −10.072 | 1.00 | 92.03 | C | |
| ATOM | 9689 | CZ | TYR | C | 312 | 38.968 | −8.745 | −10.027 | 1.00 | 92.15 | C | |
| ATOM | 9690 | OH | TYR | C | 312 | 38.771 | −9.973 | −10.618 | 1.00 | 91.23 | O | |
| ATOM | 9692 | CE2 | TYR | C | 312 | 40.190 | −8.440 | −9.388 | 1.00 | 91.24 | C | |
| ATOM | 9694 | CD2 | TYR | C | 312 | 40.358 | −7.172 | −8.788 | 1.00 | 90.88 | C | |
| ATOM | 9696 | C | TYR | C | 312 | 39.170 | −2.433 | −7.808 | 1.00 | 90.16 | C | |
| ATOM | 9697 | O | TYR | C | 312 | 40.242 | −1.808 | −7.867 | 1.00 | 90.00 | O | |
| ATOM | 9699 | N | ILE | C | 313 | 38.194 | −2.176 | −6.924 | 1.00 | 89.63 | N | |
| ATOM | 9700 | CA | ILE | C | 313 | 38.387 | −1.366 | −5.682 | 1.00 | 89.39 | C | |
| ATOM | 9702 | CE | ILE | C | 313 | 37.358 | −0.178 | −5.591 | 1.00 | 89.36 | C | |
| ATOM | 9704 | CG1 | ILE | C | 313 | 37.629 | 0.838 | −6.735 | 1.00 | 89.57 | C | |
| ATOM | 9707 | CD1 | ILE | C | 313 | 36.619 | 2.005 | −6.926 | 1.00 | 89.51 | C | |
| ATOM | 9711 | CG2 | ILE | C | 313 | 37.443 | 0.498 | −4.208 | 1.00 | 89.15 | C | |
| ATOM | 9715 | C | ILE | C | 313 | 38.311 | −2.311 | −4.454 | 1.00 | 89.06 | C | |
| ATOM | 9716 | O | ILE | C | 313 | 37.475 | −3.207 | −4.453 | 1.00 | 89.05 | O | |
| ATOM | 9718 | N | ILE | C | 314 | 39.172 | −2.079 | −3.438 | 1.00 | 88.44 | N | |
| ATOM | 9719 | CA | ILE | C | 314 | 39.324 | −2.934 | −2.224 | 1.00 | 87.99 | C | |
| ATOM | 9721 | CE | ILE | C | 314 | 40.779 | −3.598 | −2.172 | 1.00 | 87.83 | C | |
| ATOM | 9723 | CG1 | ILE | C | 314 | 41.060 | −4.429 | −3.441 | 1.00 | 86.23 | C | |
| ATOM | 9726 | CD1 | ILE | C | 314 | 41.724 | −3.697 | −4.562 | 1.00 | 84.95 | C | |
| ATOM | 9730 | CG2 | ILE | C | 314 | 40.951 | −4.475 | −0.887 | 1.00 | 88.16 | C | |
| ATOM | 9734 | C | ILE | C | 314 | 39.003 | −2.136 | −0.921 | 1.00 | 87.48 | C | |
| ATOM | 9735 | O | ILE | C | 314 | 39.612 | −1.108 | −0.644 | 1.00 | 87.48 | O | |
| ATOM | 9737 | N | THR | C | 315 | 38.040 | −2.620 | −0.141 | 1.00 | 87.12 | N | |
| ATOM | 9738 | CA | THR | C | 315 | 37.628 | −1.978 | 1.120 | 1.00 | 86.89 | C | |
| ATOM | 9740 | CB | THR | C | 315 | 36.275 | −1.256 | 0.960 | 1.00 | 86.53 | C | |
| ATOM | 9742 | OG1 | THR | C | 315 | 35.257 | −2.195 | 0.584 | 1.00 | 86.33 | O | |
| ATOM | 9744 | CG2 | THR | C | 315 | 36.294 | −0.257 | −0.170 | 1.00 | 86.32 | C | |
| ATOM | 9748 | C | THR | C | 315 | 37.534 | −3.012 | 2.246 | 1.00 | 86.80 | C | |
| ATOM | 9749 | O | THR | C | 315 | 37.877 | −4.179 | 2.051 | 1.00 | 86.76 | O | |
| ATOM | 9751 | N | GLU | C | 316 | 37.113 | −2.571 | 3.433 | 1.00 | 86.79 | N | |
| ATOM | 9752 | CA | GLU | C | 316 | 36.806 | −3.495 | 4.538 | 1.00 | 86.54 | C | |
| ATOM | 9754 | CB | GLU | C | 316 | 36.905 | −2.827 | 5.916 | 1.00 | 86.52 | C | |
| ATOM | 9757 | CG | GLU | C | 316 | 35.863 | −1.772 | 6.245 | 1.00 | 86.61 | C | |
| ATOM | 9760 | CD | GLU | C | 316 | 36.200 | −1.022 | 7.517 | 1.00 | 86.68 | C | |
| ATOM | 9761 | OE1 | GLU | C | 316 | 36.892 | −1.616 | 8.394 | 1.00 | 85.47 | O | |
| ATOM | 9762 | OE2 | GLU | C | 316 | 35.777 | 0.158 | 7.624 | 1.00 | 85.42 | O | |
| ATOM | 9763 | C | GLU | C | 316 | 35.442 | −4.155 | 4.366 | 1.00 | 86.06 | C | |
| ATOM | 9764 | O | GLU | C | 316 | 34.471 | −3.507 | 3.953 | 1.00 | 86.19 | O | |
| ATOM | 9766 | N | PHE | C | 317 | 35.392 | −5.443 | 4.709 | 1.00 | 85.49 | N | |
| ATOM | 9767 | CA | PHE | C | 317 | 34.172 | −6.230 | 4.678 | 1.00 | 84.92 | C | |
| ATOM | 9769 | CB | PHE | C | 317 | 34.495 | −7.745 | 4.633 | 1.00 | 84.90 | C | |
| ATOM | 9772 | CG | PHE | C | 317 | 33.302 | −8.621 | 4.311 | 1.00 | 85.05 | C | |
| ATOM | 9773 | CD1 | PHE | C | 317 | 32.553 | −8.403 | 3.135 | 1.00 | 85.38 | C | |
| ATOM | 9775 | CE1 | PHE | C | 317 | 31.431 | −9.200 | 2.826 | 1.00 | 85.11 | C | |
| ATOM | 9777 | CZ | PHE | C | 317 | 31.062 | −10.249 | 3.684 | 1.00 | 85.25 | C | |
| ATOM | 9779 | CE2 | PHE | C | 317 | 31.814 | −10.487 | 4.870 | 1.00 | 84.79 | C | |
| ATOM | 9781 | CD2 | PHE | C | 317 | 32.927 | −9.674 | 5.172 | 1.00 | 84.54 | C | |
| ATOM | 9783 | C | PHE | C | 317 | 33.376 | −5.846 | 5.923 | 1.00 | 84.24 | C | |
| ATOM | 9784 | O | PHE | C | 317 | 33.946 | −5.733 | 7.003 | 1.00 | 83.85 | O | |
| ATOM | 9786 | N | MET | C | 318 | 32.087 | −5.570 | 5.731 | 1.00 | 83.83 | N | |
| ATOM | 9787 | CA | MET | C | 318 | 31.137 | −5.298 | 6.798 | 1.00 | 83.70 | C | |
| ATOM | 9789 | CB | MET | C | 318 | 30.418 | −3.955 | 6.565 | 1.00 | 83.48 | C | |
| ATOM | 9792 | CG | MET | C | 318 | 31.346 | −2.682 | 6.394 | 1.00 | 83.11 | C | |
| ATOM | 9795 | SD | MET | C | 318 | 32.376 | −2.013 | 7.823 | 1.00 | 81.83 | S | |
| ATOM | 9796 | CE | MET | C | 318 | 31.217 | −2.174 | 9.211 | 1.00 | 81.59 | C | |
| ATOM | 9800 | C | MET | C | 318 | 30.167 | −6.495 | 6.759 | 1.00 | 83.94 | C | |
| ATOM | 9801 | O | MET | C | 318 | 29.463 | −6.693 | 5.748 | 1.00 | 84.32 | O | |
| ATOM | 9803 | N | THR | C | 319 | 30.142 | −7.261 | 7.861 | 1.00 | 83.69 | N | |
| ATOM | 9804 | CA | THR | C | 319 | 29.595 | −8.639 | 7.931 | 1.00 | 83.56 | C | |
| ATOM | 9806 | CB | THR | C | 319 | 29.991 | −9.265 | 9.310 | 1.00 | 83.70 | C | |
| ATOM | 9808 | OG1 | THR | C | 319 | 31.420 | −9.355 | 9.387 | 1.00 | 83.22 | O | |
| ATOM | 9810 | CG2 | THR | C | 319 | 29.516 | −10.729 | 9.486 | 1.00 | 83.74 | C | |
| ATOM | 9814 | C | THR | C | 319 | 28.087 | −8.825 | 7.683 | 1.00 | 83.59 | C | |
| ATOM | 9815 | O | THR | C | 319 | 27.685 | −9.863 | 7.136 | 1.00 | 83.60 | O | |
| ATOM | 9817 | N | TYR | C | 320 | 27.268 | −7.852 | 8.091 | 1.00 | 83.42 | N | |
| ATOM | 9818 | CA | TYR | C | 320 | 25.806 | −7.958 | 7.978 | 1.00 | 83.17 | C | |
| ATOM | 9820 | CB | TYR | C | 320 | 25.157 | −7.657 | 9.333 | 1.00 | 83.46 | C | |
| ATOM | 9823 | CG | TYR | C | 320 | 25.235 | −8.774 | 10.363 | 1.00 | 84.01 | C | |
| ATOM | 9824 | CD1 | TYR | C | 320 | 24.911 | −10.111 | 10.030 | 1.00 | 84.90 | C | |
| ATOM | 9826 | CE1 | TYR | C | 320 | 24.960 | −11.154 | 11.001 | 1.00 | 84.37 | C | |
| ATOM | 9828 | CZ | TYR | C | 320 | 25.306 | −10.855 | 12.322 | 1.00 | 84.33 | C | |
| ATOM | 9829 | OH | TYR | C | 320 | 25.341 | −11.873 | 13.248 | 1.00 | 83.75 | O | |
| ATOM | 9831 | CE2 | TYR | C | 320 | 25.614 | −9.530 | 12.689 | 1.00 | 84.41 | C | |
| ATOM | 9833 | CD2 | TYR | C | 320 | 25.574 | −8.496 | 11.707 | 1.00 | 84.47 | C | |
| ATOM | 9835 | C | TYR | C | 320 | 25.142 | −7.144 | 6.847 | 1.00 | 83.05 | C | |
| ATOM | 9836 | O | TYR | C | 320 | 23.930 | −7.282 | 6.637 | 1.00 | 83.20 | O | |
| ATOM | 9838 | N | GLY | C | 321 | 25.896 | −6.322 | 6.115 | 1.00 | 82.56 | N | |
| ATOM | 9839 | CA | GLY | C | 321 | 25.395 | −5.753 | 4.846 | 1.00 | 82.09 | C | |
| ATOM | 9842 | C | GLY | C | 321 | 24.358 | −4.640 | 4.957 | 1.00 | 81.49 | C | |
| ATOM | 9843 | O | GLY | C | 321 | 24.407 | −3.865 | 5.901 | 1.00 | 81.37 | O | |
| ATOM | 9845 | N | ASN | C | 322 | 23.409 | −4.585 | 4.008 | 1.00 | 81.02 | N | |
| ATOM | 9846 | CA | ASN | C | 322 | 22.479 | −3.429 | 3.866 | 1.00 | 80.75 | C | |
| ATOM | 9848 | CB | ASN | C | 322 | 21.835 | −3.339 | 2.465 | 1.00 | 80.57 | C | |
| ATOM | 9851 | CG | ASN | C | 322 | 20.545 | −4.113 | 2.350 | 1.00 | 80.95 | C | |
| ATOM | 9852 | OD1 | ASN | C | 322 | 19.456 | −3.541 | 2.458 | 1.00 | 80.49 | O | |
| ATOM | 9853 | ND2 | ASN | C | 322 | 20.656 | −5.427 | 2.177 | 1.00 | 81.05 | N | |
| ATOM | 9856 | C | ASN | C | 322 | 21.413 | −3.251 | 4.981 | 1.00 | 80.02 | C | |
| ATOM | 9857 | O | ASN | C | 322 | 20.684 | −4.195 | 5.381 | 1.00 | 80.29 | O | |
| ATOM | 9859 | N | LEU | C | 323 | 21.312 | −2.015 | 5.445 | 1.00 | 79.02 | N | |
| ATOM | 9860 | CA | LEU | C | 323 | 20.506 | −1.684 | 6.597 | 1.00 | 78.19 | C | |
| ATOM | 9862 | CB | LEU | C | 323 | 20.880 | −0.296 | 7.089 | 1.00 | 77.86 | C | |
| ATOM | 9865 | CG | LEU | C | 323 | 20.253 | 0.236 | 8.348 | 1.00 | 77.61 | C | |
| ATOM | 9867 | CD1 | LEU | C | 323 | 20.475 | −0.736 | 9.479 | 1.00 | 78.62 | C | |
| ATOM | 9871 | CD2 | LEU | C | 323 | 20.857 | 1.583 | 8.661 | 1.00 | 77.66 | C | |
| ATOM | 9875 | C | LEU | C | 323 | 19.001 | −1.797 | 6.329 | 1.00 | 78.14 | C | |
| ATOM | 9876 | O | LEU | C | 323 | 18.295 | −2.294 | 7.187 | 1.00 | 77.83 | O | |
| ATOM | 9878 | N | LEU | C | 324 | 18.528 | −1.403 | 5.135 | 1.00 | 78.17 | N | |
| ATOM | 9879 | CA | LEU | C | 324 | 17.086 | −1.479 | 4.784 | 1.00 | 78.17 | C | |
| ATOM | 9881 | CB | LEU | C | 324 | 16.827 | −1.074 | 3.333 | 1.00 | 77.78 | C | |
| ATOM | 9884 | CG | LEU | C | 324 | 15.365 | −1.026 | 2.833 | 1.00 | 78.07 | C | |
| ATOM | 9886 | CD1 | LEU | C | 324 | 14.469 | −0.175 | 3.718 | 1.00 | 79.07 | C | |
| ATOM | 9890 | CD2 | LEU | C | 324 | 15.282 | −0.487 | 1.410 | 1.00 | 78.50 | C | |
| ATOM | 9894 | C | LEU | C | 324 | 16.502 | −2.870 | 4.997 | 1.00 | 78.66 | C | |
| ATOM | 9895 | O | LEU | C | 324 | 15.457 | −3.018 | 5.631 | 1.00 | 78.63 | O | |
| ATOM | 9897 | N | ASP | C | 325 | 17.193 | −3.877 | 4.465 | 1.00 | 79.37 | N | |
| ATOM | 9898 | CA | ASP | C | 325 | 16.776 | −5.271 | 4.605 | 1.00 | 79.67 | C | |
| ATOM | 9900 | CB | ASP | C | 325 | 17.438 | −6.167 | 3.551 | 1.00 | 79.61 | C | |
| ATOM | 9903 | CG | ASP | C | 325 | 16.722 | −6.108 | 2.200 | 1.00 | 79.91 | C | |
| ATOM | 9904 | OD1 | ASP | C | 325 | 15.468 | −6.143 | 2.187 | 1.00 | 79.60 | O | |
| ATOM | 9905 | OD2 | ASP | C | 325 | 17.321 | −6.040 | 1.095 | 1.00 | 79.90 | O | |
| ATOM | 9906 | C | ASP | C | 325 | 17.019 | −5.779 | 6.016 | 1.00 | 80.19 | C | |
| ATOM | 9907 | O | ASP | C | 325 | 16.120 | −6.397 | 6.603 | 1.00 | 80.11 | O | |
| ATOM | 9909 | N | TYR | C | 326 | 18.192 | −5.477 | 6.578 | 1.00 | 80.58 | N | |
| ATOM | 9910 | CA | TYR | C | 326 | 18.494 | −5.820 | 7.986 | 1.00 | 80.89 | C | |
| ATOM | 9912 | CB | TYR | C | 326 | 19.780 | −5.127 | 8.424 | 1.00 | 81.17 | C | |
| ATOM | 9915 | CG | TYR | C | 326 | 20.129 | −5.348 | 9.861 | 1.00 | 81.76 | C | |
| ATOM | 9916 | CD1 | TYR | C | 326 | 20.414 | −6.632 | 10.339 | 1.00 | 82.98 | C | |
| ATOM | 9918 | CE1 | TYR | C | 326 | 20.742 | −6.857 | 11.696 | 1.00 | 82.86 | C | |
| ATOM | 9920 | CZ | TYR | C | 326 | 20.786 | −5.773 | 12.576 | 1.00 | 82.65 | C | |
| ATOM | 9921 | OH | TYR | C | 326 | 21.110 | −5.955 | 13.897 | 1.00 | 82.36 | O | |
| ATOM | 9923 | CE2 | TYR | C | 326 | 20.501 | −4.486 | 12.116 | 1.00 | 83.06 | C | |
| ATOM | 9925 | CD2 | TYR | C | 326 | 20.171 | −4.282 | 10.763 | 1.00 | 83.08 | C | |
| ATOM | 9927 | C | TYR | C | 326 | 17.346 | −5.487 | 8.962 | 1.00 | 80.83 | C | |
| ATOM | 9928 | O | TYR | C | 326 | 16.886 | −6.353 | 9.691 | 1.00 | 80.90 | O | |
| ATOM | 9930 | N | LEU | C | 327 | 16.865 | −4.248 | 8.899 | 1.00 | 80.99 | N | |
| ATOM | 9931 | CA | LEU | C | 327 | 15.818 | −3.747 | 9.790 | 1.00 | 80.99 | C | |
| ATOM | 9933 | CB | LEU | C | 327 | 15.716 | −2.224 | 9.710 | 1.00 | 80.94 | C | |
| ATOM | 9936 | CG | LEU | C | 327 | 16.940 | −1.388 | 10.066 | 1.00 | 80.79 | C | |
| ATOM | 9938 | CD1 | LEU | C | 327 | 16.554 | 0.115 | 9.947 | 1.00 | 80.32 | C | |
| ATOM | 9942 | CD2 | LEU | C | 327 | 17.565 | −1.734 | 11.426 | 1.00 | 79.34 | C | |
| ATOM | 9946 | C | LEU | C | 327 | 14.425 | −4.301 | 9.564 | 1.00 | 81.13 | C | |
| ATOM | 9947 | O | LEU | C | 327 | 13.681 | −4.459 | 10.525 | 1.00 | 81.44 | O | |
| ATOM | 9949 | N | ARG | C | 328 | 14.048 | −4.549 | 8.314 | 1.00 | 81.25 | N | |
| ATOM | 9950 | CA | ARG | C | 328 | 12.784 | −5.263 | 8.019 | 1.00 | 81.26 | C | |
| ATOM | 9952 | CB | ARG | C | 328 | 12.502 | −5.295 | 6.508 | 1.00 | 81.52 | C | |
| ATOM | 9955 | CG | ARG | C | 328 | 12.184 | −3.983 | 5.867 | 1.00 | 81.59 | C | |
| ATOM | 9958 | CD | ARG | C | 328 | 12.262 | −4.028 | 4.326 | 1.00 | 81.90 | C | |
| ATOM | 9961 | NE | ARG | C | 328 | 11.474 | −2.945 | 3.734 | 1.00 | 82.65 | N | |
| ATOM | 9963 | CZ | ARG | C | 328 | 11.581 | −2.467 | 2.493 | 1.00 | 83.03 | C | |
| ATOM | 9964 | NH1 | ARG | C | 328 | 12.429 | −2.992 | 1.604 | 1.00 | 84.29 | N | |
| ATOM | 9967 | NH2 | ARG | C | 328 | 10.793 | −1.449 | 2.127 | 1.00 | 82.82 | N | |
| ATOM | 9970 | C | ARG | C | 328 | 12.744 | −6.722 | 8.537 | 1.00 | 80.91 | C | |
| ATOM | 9971 | O | ARG | C | 328 | 11.656 | −7.248 | 8.754 | 1.00 | 80.39 | O | |
| ATOM | 9973 | N | GLU | C | 329 | 13.918 | −7.350 | 8.701 | 1.00 | 80.81 | N | |
| ATOM | 9974 | CA | GLU | C | 329 | 14.063 | −8.790 | 8.965 | 1.00 | 80.82 | C | |
| ATOM | 9976 | CB | GLU | C | 329 | 14.887 | −9.419 | 7.812 | 1.00 | 80.80 | C | |
| ATOM | 9979 | CG | GLU | C | 329 | 14.088 | −9.542 | 6.498 | 1.00 | 81.37 | C | |
| ATOM | 9982 | CD | GLU | C | 329 | 14.928 | −9.587 | 5.207 | 1.00 | 81.66 | C | |
| ATOM | 9983 | OE1 | GLU | C | 329 | 15.730 | −8.671 | 4.942 | 1.00 | 82.08 | O | |
| ATOM | 9984 | OE2 | GLU | C | 329 | 14.764 | −10.539 | 4.406 | 1.00 | 84.53 | O | |
| ATOM | 9985 | C | GLU | C | 329 | 14.628 | −9.163 | 10.372 | 1.00 | 80.55 | C | |
| ATOM | 9986 | O | GLU | C | 329 | 14.541 | −10.332 | 10.772 | 1.00 | 80.06 | O | |
| ATOM | 9988 | N | CYS | C | 330 | 15.155 | −8.188 | 11.129 | 1.00 | 80.37 | N | |
| ATOM | 9989 | CA | CYS | C | 330 | 15.718 | −8.442 | 12.488 | 1.00 | 80.24 | C | |
| ATOM | 9991 | CB | CYS | C | 330 | 16.596 | −7.274 | 12.948 | 1.00 | 80.30 | C | |
| ATOM | 9994 | SG | CYS | C | 330 | 15.644 | −5.808 | 13.446 | 1.00 | 81.52 | S | |
| ATOM | 9996 | C | CYS | C | 330 | 14.676 | −8.663 | 13.589 | 1.00 | 79.48 | C | |
| ATOM | 9997 | O | CYS | C | 330 | 13.488 | −8.409 | 13.401 | 1.00 | 79.59 | O | |
| ATOM | 9999 | N | ASN | C | 331 | 15.168 | −9.104 | 14.749 | 1.00 | 78.73 | N | |
| ATOM | 10000 | CA | ASN | C | 331 | 14.391 | −9.184 | 16.008 | 1.00 | 77.75 | C | |
| ATOM | 10002 | CB | ASN | C | 331 | 14.915 | −10.353 | 16.899 | 1.00 | 77.66 | C | |
| ATOM | 10005 | CG | ASN | C | 331 | 14.625 | −10.167 | 18.386 | 1.00 | 77.54 | C | |
| ATOM | 10006 | OD1 | ASN | C | 331 | 15.512 | −10.333 | 9.220 | 1.00 | 77.10 | O | |
| ATOM | 10007 | ND2 | ASN | C | 331 | 13.391 | −9.817 | 18.720 | 1.00 | 77.59 | N | |
| ATOM | 10010 | C | ASN | C | 331 | 14.448 | −7.796 | 16.691 | 1.00 | 77.16 | C | |
| ATOM | 10011 | O | ASN | C | 331 | 15.529 | −7.347 | 17.117 | 1.00 | 76.53 | O | |
| ATOM | 10013 | N | ARG | C | 332 | 13.284 | −7.142 | 16.796 | 1.00 | 76.69 | N | |
| ATOM | 10014 | CA | ARG | C | 332 | 13.203 | −5.711 | 17.226 | 1.00 | 76.31 | C | |
| ATOM | 10016 | CB | ARG | C | 332 | 11.859 | −5.081 | 16.828 | 1.00 | 75.91 | C | |
| ATOM | 10019 | CG | ARG | C | 332 | 11.649 | −4.872 | 15.325 | 1.00 | 75.57 | C | |
| ATOM | 10022 | CD | ARG | C | 332 | 10.316 | −4.214 | 15.008 | 1.00 | 74.80 | C | |
| ATOM | 10025 | NE | ARG | C | 332 | 9.983 | −4.122 | 13.586 | 1.00 | 73.35 | N | |
| ATOM | 10027 | CZ | ARG | C | 332 | 8.838 | −3.614 | 13.084 | 1.00 | 71.59 | C | |
| ATOM | 10028 | NH1 | ARG | C | 332 | 7.871 | −3.132 | 13.873 | 1.00 | 69.71 | N | |
| ATOM | 10031 | NH2 | ARG | C | 332 | 8.655 | −3.578 | 11.764 | 1.00 | 72.20 | N | |
| ATOM | 10034 | C | ARG | C | 332 | 13.450 | −5.468 | 18.724 | 1.00 | 76.10 | C | |
| ATOM | 10035 | O | ARG | C | 332 | 13.802 | −4.362 | 19.115 | 1.00 | 75.59 | O | |
| ATOM | 10037 | N | GLN | C | 333 | 13.277 | −6.502 | 19.547 | 1.00 | 76.33 | N | |
| ATOM | 10038 | CA | GLN | C | 333 | 13.566 | −6.418 | 20.996 | 1.00 | 76.44 | C | |
| ATOM | 10040 | CB | GLN | C | 333 | 13.025 | −7.623 | 21.833 | 1.00 | 76.35 | C | |
| ATOM | 10043 | CG | GLN | C | 333 | 11.688 | −8.350 | 21.423 | 1.00 | 75.65 | C | |
| ATOM | 10046 | CD | GLN | C | 333 | 10.584 | −7.486 | 20.776 | 1.00 | 74.62 | C | |
| ATOM | 10047 | OE1 | GLN | C | 333 | 10.422 | −6.299 | 21.073 | 1.00 | 72.82 | O | |
| ATOM | 10048 | NE2 | GLN | C | 333 | 9.822 | −8.111 | 19.889 | 1.00 | 74.15 | N | |
| ATOM | 10051 | C | GLN | C | 333 | 15.087 | −6.269 | 21.203 | 1.00 | 76.50 | C | |
| ATOM | 10052 | O | GLN | C | 333 | 15.529 | −5.465 | 22.034 | 1.00 | 76.59 | O | |
| ATOM | 10054 | N | GLU | C | 334 | 15.862 | −7.050 | 20.442 | 1.00 | 76.41 | N | |
| ATOM | 10055 | CA | GLU | C | 334 | 17.320 | −6.913 | 20.396 | 1.00 | 76.20 | C | |
| ATOM | 10057 | CB | GLU | C | 334 | 17.939 | −8.078 | 19.623 | 1.00 | 76.25 | C | |
| ATOM | 10060 | CG | GLU | C | 334 | 19.451 | −8.183 | 19.705 | 1.00 | 76.71 | C | |
| ATOM | 10063 | CD | GLU | C | 334 | 20.020 | −9.130 | 18.658 | 1.00 | 76.69 | C | |
| ATOM | 10064 | OE1 | GLU | C | 334 | 19.728 | −10.345 | 18.756 | 1.00 | 77.74 | O | |
| ATOM | 10065 | OE2 | GLU | C | 334 | 20.754 | −8.664 | 17.747 | 1.00 | 76.51 | O | |
| ATOM | 10066 | C | GLU | C | 334 | 17.712 | −5.560 | 19.781 | 1.00 | 75.98 | C | |
| ATOM | 10067 | O | GLU | C | 334 | 18.501 | −4.824 | 20.381 | 1.00 | 76.15 | O | |
| ATOM | 10069 | N | VAL | C | 335 | 17.130 | −5.215 | 18.629 | 1.00 | 75.62 | N | |
| ATOM | 10070 | CA | VAL | C | 335 | 17.397 | −3.915 | 17.952 | 1.00 | 75.32 | C | |
| ATOM | 10072 | CB | VAL | C | 335 | 17.297 | −4.066 | 16.375 | 1.00 | 75.27 | C | |
| ATOM | 10074 | CG1 | VAL | C | 335 | 17.718 | −2.807 | 15.658 | 1.00 | 75.23 | C | |
| ATOM | 10078 | CG2 | VAL | C | 335 | 18.180 | −5.229 | 15.866 | 1.00 | 75.52 | C | |
| ATOM | 10082 | C | VAL | C | 335 | 16.482 | −2.791 | 18.547 | 1.00 | 75.06 | C | |
| ATOM | 10083 | O | VAL | C | 335 | 15.638 | −2.212 | 17.867 | 1.00 | 75.10 | O | |
| ATOM | 10085 | N | ASN | C | 336 | 16.674 | −2.490 | 19.832 | 1.00 | 74.73 | N | |
| ATOM | 10086 | CA | ASN | C | 336 | 15.763 | −1.607 | 20.611 | 1.00 | 74.22 | C | |
| ATOM | 10088 | CE | ASN | C | 336 | 15.884 | −1.893 | 22.138 | 1.00 | 74.35 | C | |
| ATOM | 10091 | CG | ASN | C | 336 | 17.267 | −1.521 | 22.738 | 1.00 | 75.94 | C | |
| ATOM | 10092 | OD1 | ASN | C | 336 | 17.971 | −0.647 | 22.236 | 1.00 | 79.80 | O | |
| ATOM | 10093 | ND2 | ASN | C | 336 | 17.647 | −2.203 | 23.820 | 1.00 | 78.06 | N | |
| ATOM | 10096 | C | ASN | C | 336 | 15.973 | −0.123 | 20.314 | 1.00 | 73.19 | C | |
| ATOM | 10097 | O | ASN | C | 336 | 16.865 | 0.224 | 19.570 | 1.00 | 72.58 | O | |
| ATOM | 10099 | N | ALA | C | 337 | 15.186 | 0.734 | 20.956 | 1.00 | 72.76 | N | |
| ATOM | 10100 | CA | ALA | C | 337 | 15.293 | 2.203 | 20.813 | 1.00 | 73.06 | C | |
| ATOM | 10102 | CB | ALA | C | 337 | 14.387 | 2.911 | 21.840 | 1.00 | 72.88 | C | |
| ATOM | 10106 | C | ALA | C | 337 | 16.707 | 2.766 | 20.913 | 1.00 | 72.75 | C | |
| ATOM | 10107 | O | ALA | C | 337 | 17.073 | 3.670 | 20.148 | 1.00 | 72.35 | O | |
| ATOM | 10109 | N | VAL | C | 338 | 17.480 | 2.210 | 21.847 | 1.00 | 72.56 | N | |
| ATOM | 10110 | CA | VAL | C | 338 | 18.890 | 2.591 | 22.063 | 1.00 | 72.58 | C | |
| ATOM | 10112 | CE | VAL | C | 338 | 19.493 | 1.957 | 23.383 | 1.00 | 72.88 | C | |
| ATOM | 10114 | CG1 | VAL | C | 338 | 20.966 | 2.362 | 23.575 | 1.00 | 73.53 | C | |
| ATOM | 10118 | CG2 | VAL | C | 338 | 18.665 | 2.365 | 24.613 | 1.00 | 73.40 | C | |
| ATOM | 10122 | C | VAL | C | 338 | 19.802 | 2.245 | 20.859 | 1.00 | 72.27 | C | |
| ATOM | 10123 | O | VAL | C | 338 | 20.727 | 3.014 | 20.548 | 1.00 | 71.86 | O | |
| ATOM | 10125 | N | VAL | C | 339 | 19.529 | 1.110 | 20.200 | 1.00 | 71.90 | N | |
| ATOM | 10126 | CA | VAL | C | 339 | 20.356 | 0.604 | 19.082 | 1.00 | 71.33 | C | |
| ATOM | 10128 | CE | VAL | C | 339 | 20.026 | −0.892 | 18.716 | 1.00 | 71.06 | C | |
| ATOM | 10130 | CG1 | VAL | C | 339 | 20.961 | −1.414 | 17.614 | 1.00 | 71.19 | C | |
| ATOM | 10134 | CG2 | VAL | C | 339 | 20.111 | −1.808 | 19.923 | 1.00 | 70.45 | C | |
| ATOM | 10138 | C | VAL | C | 339 | 20.169 | 1.534 | 17.857 | 1.00 | 71.09 | C | |
| ATOM | 10139 | O | VAL | C | 339 | 21.152 | 2.029 | 17.296 | 1.00 | 70.89 | O | |
| ATOM | 10141 | N | LEU | C | 340 | 18.906 | 1.790 | 17.492 | 1.00 | 70.69 | N | |
| ATOM | 10142 | CA | LEU | C | 340 | 18.519 | 2.804 | 16.472 | 1.00 | 70.43 | C | |
| ATOM | 10144 | CE | LEU | C | 340 | 16.995 | 2.970 | 16.406 | 1.00 | 70.31 | C | |
| ATOM | 10147 | CG | LEU | C | 340 | 16.056 | 1.753 | 16.257 | 1.00 | 69.85 | C | |
| ATOM | 10149 | CD1 | LEU | C | 340 | 14.685 | 2.196 | 15.764 | 1.00 | 69.59 | C | |
| ATOM | 10153 | CD2 | LEU | C | 340 | 16.609 | 0.664 | 15.349 | 1.00 | 68.71 | C | |
| ATOM | 10157 | C | LEU | C | 340 | 19.129 | 4.184 | 16.726 | 1.00 | 70.14 | C | |
| ATOM | 10158 | O | LEU | C | 340 | 19.513 | 4.877 | 15.801 | 1.00 | 70.05 | O | |
| ATOM | 10160 | N | LEU | C | 341 | 19.214 | 4.551 | 17.999 | 1.00 | 70.16 | N | |
| ATOM | 10161 | CA | LEU | C | 341 | 19.878 | 5.783 | 18.433 | 1.00 | 70.26 | C | |
| ATOM | 10163 | CE | LEU | C | 341 | 19.663 | 6.006 | 19.937 | 1.00 | 70.28 | C | |
| ATOM | 10166 | CG | LEU | C | 341 | 19.355 | 7.363 | 20.574 | 1.00 | 70.49 | C | |
| ATOM | 10168 | CD1 | LEU | C | 341 | 18.625 | 8.345 | 19.687 | 1.00 | 70.21 | C | |
| ATOM | 10172 | CD2 | LEU | C | 341 | 18.556 | 7.138 | 21.884 | 1.00 | 70.41 | C | |
| ATOM | 10176 | C | LEU | C | 341 | 21.358 | 5.748 | 18.127 | 1.00 | 70.36 | C | |
| ATOM | 10177 | O | LEU | C | 341 | 21.909 | 6.762 | 17.709 | 1.00 | 70.74 | O | |
| ATOM | 10179 | N | TYR | C | 342 | 21.981 | 4.581 | 18.337 | 1.00 | 70.76 | N | |
| ATOM | 10180 | CA | TYR | C | 342 | 23.438 | 4.360 | 18.135 | 1.00 | 70.71 | C | |
| ATOM | 10182 | CE | TYR | C | 342 | 23.887 | 3.049 | 18.842 | 1.00 | 70.78 | C | |
| ATOM | 10185 | CG | TYR | C | 342 | 25.372 | 2.652 | 18.725 | 1.00 | 71.23 | C | |
| ATOM | 10186 | CD1 | TYR | C | 342 | 26.417 | 3.570 | 19.031 | 1.00 | 71.51 | C | |
| ATOM | 10188 | CE1 | TYR | C | 342 | 27.794 | 3.201 | 18.909 | 1.00 | 70.48 | C | |
| ATOM | 10190 | CZ | TYR | C | 342 | 28.124 | 1.903 | 18.498 | 1.00 | 71.24 | C | |
| ATOM | 10191 | OH | TYR | C | 342 | 29.446 | 1.519 | 18.373 | 1.00 | 70.81 | O | |
| ATOM | 10193 | CE2 | TYR | C | 342 | 27.112 | 0.974 | 18.202 | 1.00 | 71.30 | C | |
| ATOM | 10195 | CD2 | TYR | C | 342 | 25.743 | 1.343 | 18.323 | 1.00 | 70.62 | C | |
| ATOM | 10197 | C | TYR | C | 342 | 23.792 | 4.358 | 16.646 | 1.00 | 70.80 | C | |
| ATOM | 10198 | O | TYR | C | 342 | 24.716 | 5.074 | 16.215 | 1.00 | 70.45 | O | |
| ATOM | 10200 | N | MET | C | 343 | 23.021 | 3.587 | 15.870 | 1.00 | 70.97 | N | |
| ATOM | 10201 | CA | MET | C | 343 | 23.162 | 3.515 | 14.390 | 1.00 | 70.93 | C | |
| ATOM | 10203 | CE | MET | C | 343 | 22.140 | 2.545 | 13.770 | 1.00 | 70.47 | C | |
| ATOM | 10206 | CG | MET | C | 343 | 22.282 | 1.068 | 14.308 | 1.00 | 71.90 | C | |
| ATOM | 10209 | SD | MET | C | 343 | 21.151 | −0.272 | 13.701 | 1.00 | 72.79 | S | |
| ATOM | 10210 | CE | MET | C | 343 | 19.689 | 0.704 | 13.188 | 1.00 | 72.30 | C | |
| ATOM | 10214 | C | MET | C | 343 | 23.047 | 4.912 | 13.780 | 1.00 | 70.21 | C | |
| ATOM | 10215 | O | MET | C | 343 | 23.850 | 5.283 | 12.959 | 1.00 | 70.22 | O | |
| ATOM | 10217 | N | ALA | C | 344 | 22.092 | 5.701 | 14.255 | 1.00 | 70.12 | N | |
| ATOM | 10218 | CA | ALA | C | 344 | 21.969 | 7.125 | 13.886 | 1.00 | 70.11 | C | |
| ATOM | 10220 | CB | ALA | C | 344 | 20.672 | 7.692 | 14.444 | 1.00 | 70.28 | C | |
| ATOM | 10224 | C | ALA | C | 344 | 23.131 | 8.006 | 14.318 | 1.00 | 69.84 | C | |
| ATOM | 10225 | O | ALA | C | 344 | 23.493 | 8.930 | 13.616 | 1.00 | 69.08 | O | |
| ATOM | 10227 | N | THR | C | 345 | 23.691 | 7.738 | 15.485 | 1.00 | 70.52 | N | |
| ATOM | 10228 | CA | THR | C | 345 | 24.843 | 8.521 | 15.972 | 1.00 | 71.46 | C | |
| ATOM | 10230 | CB | THR | C | 345 | 25.074 | 8.262 | 17.470 | 1.00 | 71.52 | C | |
| ATOM | 10232 | OG1 | THR | C | 345 | 23.839 | 8.468 | 18.174 | 1.00 | 71.80 | O | |
| ATOM | 10234 | CG2 | THR | C | 345 | 26.024 | 9.311 | 18.080 | 1.00 | 71.23 | C | |
| ATOM | 10238 | C | THR | C | 345 | 26.133 | 8.289 | 15.159 | 1.00 | 71.79 | C | |
| ATOM | 10239 | O | THR | C | 345 | 26.860 | 9.237 | 14.857 | 1.00 | 71.96 | O | |
| ATOM | 10241 | N | GLN | C | 346 | 26.382 | 7.034 | 14.801 | 1.00 | 72.01 | N | |
| ATOM | 10242 | CA | GLN | C | 346 | 27.511 | 6.654 | 13.944 | 1.00 | 72.36 | C | |
| ATOM | 10244 | CB | GLN | C | 346 | 27.539 | 5.134 | 13.830 | 1.00 | 72.56 | C | |
| ATOM | 10247 | CG | GLN | C | 346 | 27.932 | 4.469 | 15.128 | 1.00 | 73.45 | C | |
| ATOM | 10250 | CD | GLN | C | 346 | 27.904 | 2.990 | 15.014 | 1.00 | 73.76 | C | |
| ATOM | 10251 | OE1 | GLN | C | 346 | 28.916 | 2.387 | 14.674 | 1.00 | 73.65 | O | |
| ATOM | 10252 | NE2 | GLN | C | 346 | 26.740 | 2.394 | 15.236 | 1.00 | 73.05 | N | |
| ATOM | 10255 | C | GLN | C | 346 | 27.494 | 7.278 | 12.523 | 1.00 | 72.65 | C | |
| ATOM | 10256 | O | GLN | C | 346 | 28.533 | 7.702 | 11.971 | 1.00 | 72.77 | O | |
| ATOM | 10258 | N | ILE | C | 347 | 26.304 | 7.328 | 11.944 | 1.00 | 72.39 | N | |
| ATOM | 10259 | CA | ILE | C | 347 | 26.118 | 7.905 | 10.630 | 1.00 | 72.43 | C | |
| ATOM | 10261 | CB | ILE | C | 347 | 24.815 | 7.336 | 9.978 | 1.00 | 72.24 | C | |
| ATOM | 10263 | CG1 | ILE | C | 347 | 25.001 | 5.817 | 9.826 | 1.00 | 72.40 | C | |
| ATOM | 10266 | CD1 | ILE | C | 347 | 23.817 | 5.074 | 9.312 | 1.00 | 73.30 | C | |
| ATOM | 10270 | CG2 | ILE | C | 347 | 24.541 | 7.954 | 8.594 | 1.00 | 71.56 | C | |
| ATOM | 10274 | C | ILE | C | 347 | 26.226 | 9.449 | 10.694 | 1.00 | 72.26 | C | |
| ATOM | 10275 | O | ILE | C | 347 | 26.833 | 10.028 | 9.806 | 1.00 | 72.44 | O | |
| ATOM | 10277 | N | SER | C | 348 | 25.714 | 10.093 | 11.747 | 1.00 | 72.15 | N | |
| ATOM | 10278 | CA | SER | C | 348 | 25.851 | 11.560 | 11.911 | 1.00 | 72.19 | C | |
| ATOM | 10280 | CB | SER | C | 348 | 24.971 | 12.077 | 13.075 | 1.00 | 72.10 | C | |
| ATOM | 10283 | OG | SER | C | 348 | 25.368 | 11.574 | 14.344 | 1.00 | 71.81 | O | |
| ATOM | 10285 | C | SER | C | 348 | 27.328 | 12.074 | 12.043 | 1.00 | 72.34 | C | |
| ATOM | 10286 | O | SER | C | 348 | 27.621 | 13.241 | 11.687 | 1.00 | 72.39 | O | |
| ATOM | 10288 | N | SER | C | 349 | 28.236 | 11.208 | 12.517 | 1.00 | 72.20 | N | |
| ATOM | 10289 | CA | SER | C | 349 | 29.665 | 11.549 | 12.701 | 1.00 | 72.36 | C | |
| ATOM | 10291 | CB | SER | C | 349 | 30.323 | 10.638 | 13.752 | 1.00 | 72.44 | C | |
| ATOM | 10294 | OG | SER | C | 349 | 30.655 | 9.362 | 13.232 | 1.00 | 72.96 | O | |
| ATOM | 10296 | C | SER | C | 349 | 30.458 | 11.471 | 11.399 | 1.00 | 72.29 | C | |
| ATOM | 10297 | O | SER | C | 349 | 31.247 | 12.367 | 11.102 | 1.00 | 72.14 | O | |
| ATOM | 10299 | N | ALA | C | 350 | 30.270 | 10.376 | 10.657 | 1.00 | 72.27 | N | |
| ATOM | 10300 | CA | ALA | C | 350 | 30.787 | 10.254 | 9.283 | 1.00 | 72.61 | C | |
| ATOM | 10302 | CB | ALA | C | 350 | 30.339 | 8.953 | 8.643 | 1.00 | 72.71 | C | |
| ATOM | 10306 | C | ALA | C | 350 | 30.335 | 11.422 | 8.419 | 1.00 | 72.60 | C | |
| ATOM | 10307 | O | ALA | C | 350 | 31.152 | 12.036 | 7.743 | 1.00 | 72.58 | O | |
| ATOM | 10309 | N | MET | C | 351 | 29.041 | 11.741 | 8.483 | 1.00 | 72.70 | N | |
| ATOM | 10310 | CA | MET | C | 351 | 28.504 | 12.923 | 7.797 | 1.00 | 72.96 | C | |
| ATOM | 10312 | CB | MET | C | 351 | 26.968 | 12.955 | 7.806 | 1.00 | 73.10 | C | |
| ATOM | 10315 | CG | MET | C | 351 | 26.247 | 11.814 | 7.071 | 1.00 | 73.38 | C | |
| ATOM | 10318 | SD | MET | C | 351 | 26.945 | 11.372 | 5.455 | 1.00 | 75.16 | S | |
| ATOM | 10319 | CE | MET | C | 351 | 26.928 | 12.955 | 4.573 | 1.00 | 75.75 | C | |
| ATOM | 10323 | C | MET | C | 351 | 29.044 | 14.251 | 8.307 | 1.00 | 72.97 | C | |
| ATOM | 10324 | O | MET | C | 351 | 29.214 | 15.163 | 7.505 | 1.00 | 73.31 | O | |
| ATOM | 10326 | N | GLU | C | 352 | 29.312 | 14.375 | 9.607 | 1.00 | 72.98 | N | |
| ATOM | 10327 | CA | GLU | C | 352 | 30.055 | 15.544 | 10.107 | 1.00 | 72.93 | C | |
| ATOM | 10329 | CB | GLU | C | 352 | 30.152 | 15.584 | 11.647 | 1.00 | 73.13 | C | |
| ATOM | 10332 | CG | GLU | C | 352 | 31.057 | 16.710 | 12.172 | 1.00 | 72.88 | C | |
| ATOM | 10335 | CD | GLU | C | 352 | 30.852 | 17.082 | 13.616 | 1.00 | 73.46 | C | |
| ATOM | 10336 | OE1 | GLU | C | 352 | 30.371 | 16.263 | 14.423 | 1.00 | 75.64 | O | |
| ATOM | 10337 | OE2 | GLU | C | 352 | 31.207 | 18.224 | 13.954 | 1.00 | 75.68 | O | |
| ATOM | 10338 | C | GLU | C | 352 | 31.464 | 15.615 | 9.510 | 1.00 | 72.87 | C | |
| ATOM | 10339 | O | GLU | C | 352 | 31.947 | 16.732 | 9.244 | 1.00 | 72.91 | O | |
| ATOM | 10341 | N | TYR | C | 353 | 32.117 | 14.449 | 9.336 | 1.00 | 72.62 | N | |
| ATOM | 10342 | CA | TYR | C | 353 | 33.496 | 14.382 | 8.790 | 1.00 | 72.32 | C | |
| ATOM | 10344 | CB | TYR | C | 353 | 34.094 | 12.973 | 8.912 | 1.00 | 72.24 | C | |
| ATOM | 10347 | CG | TYR | C | 353 | 35.396 | 12.801 | 8.154 | 1.00 | 72.49 | C | |
| ATOM | 10348 | CD1 | TYR | C | 353 | 36.608 | 13.294 | 8.670 | 1.00 | 72.59 | C | |
| ATOM | 10350 | CE1 | TYR | C | 353 | 37.818 | 13.140 | 7.976 | 1.00 | 72.54 | C | |
| ATOM | 10352 | CZ | TYR | C | 353 | 37.814 | 12.499 | 6.733 | 1.00 | 73.30 | C | |
| ATOM | 10353 | OH | TYR | C | 353 | 38.986 | 12.324 | 6.016 | 1.00 | 72.72 | O | |
| ATOM | 10355 | CE2 | TYR | C | 353 | 36.614 | 12.007 | 6.204 | 1.00 | 73.19 | C | |
| ATOM | 10357 | CD2 | TYR | C | 353 | 35.418 | 12.168 | 6.914 | 1.00 | 72.62 | C | |
| ATOM | 10359 | C | TYR | C | 353 | 33.555 | 14.862 | 7.338 | 1.00 | 71.93 | C | |
| ATOM | 10360 | O | TYR | C | 353 | 34.338 | 15.753 | 7.008 | 1.00 | 71.48 | O | |
| ATOM | 10362 | N | LEU | C | 354 | 32.715 | 14.255 | 6.497 | 1.00 | 71.67 | N | |
| ATOM | 10363 | CA | LEU | C | 354 | 32.591 | 14.624 | 5.094 | 1.00 | 71.36 | C | |
| ATOM | 10365 | CB | LEU | C | 354 | 31.526 | 13.777 | 4.410 | 1.00 | 70.90 | C | |
| ATOM | 10368 | CG | LEU | C | 354 | 31.820 | 12.279 | 4.253 | 1.00 | 70.54 | C | |
| ATOM | 10370 | CD1 | LEU | C | 354 | 30.573 | 11.504 | 3.917 | 1.00 | 68.88 | C | |
| ATOM | 10374 | CD2 | LEU | C | 354 | 32.848 | 12.013 | 3.183 | 1.00 | 71.21 | C | |
| ATOM | 10378 | C | LEU | C | 354 | 32.273 | 16.113 | 4.935 | 1.00 | 71.44 | C | |
| ATOM | 10379 | O | LEU | C | 354 | 32.910 | 16.789 | 4.129 | 1.00 | 71.35 | O | |
| ATOM | 10381 | N | GLU | C | 355 | 31.329 | 16.614 | 5.730 | 1.00 | 71.78 | N | |
| ATOM | 10382 | CA | GLU | C | 355 | 30.929 | 18.043 | 5.708 | 1.00 | 72.31 | C | |
| ATOM | 10384 | CB | GLU | C | 355 | 29.827 | 18.307 | 6.747 | 1.00 | 72.29 | C | |
| ATOM | 10387 | CG | GLU | C | 355 | 29.172 | 19.691 | 6.685 | 1.00 | 72.38 | C | |
| ATOM | 10390 | CD | GLU | C | 355 | 28.545 | 20.086 | 8.015 | 1.00 | 73.12 | C | |
| ATOM | 10391 | OE1 | GLU | C | 355 | 29.304 | 20.313 | 8.990 | 1.00 | 75.54 | O | |
| ATOM | 10392 | OE2 | GLU | C | 355 | 27.305 | 20.165 | 8.093 | 1.00 | 72.31 | O | |
| ATOM | 10393 | C | GLU | C | 355 | 32.115 | 19.007 | 5.942 | 1.00 | 72.72 | C | |
| ATOM | 10394 | O | GLU | C | 355 | 32.291 | 19.999 | 5.219 | 1.00 | 72.01 | O | |
| ATOM | 10396 | N | LYS | C | 356 | 32.908 | 18.685 | 6.963 | 1.00 | 73.41 | N | |
| ATOM | 10397 | CA | LYS | C | 356 | 34.162 | 19.383 | 7.264 | 1.00 | 73.76 | C | |
| ATOM | 10399 | CB | LYS | C | 356 | 34.844 | 18.757 | 8.495 | 1.00 | 73.70 | C | |
| ATOM | 10402 | CG | LYS | C | 356 | 35.861 | 19.656 | 9.159 | 1.00 | 74.42 | C | |
| ATOM | 10405 | CD | LYS | C | 356 | 36.885 | 18.875 | 10.002 | 1.00 | 74.54 | C | |
| ATOM | 10408 | CE | LYS | C | 356 | 38.243 | 19.608 | 10.103 | 1.00 | 74.52 | C | |
| ATOM | 10411 | NZ | LYS | C | 356 | 39.325 | 18.699 | 10.610 | 1.00 | 74.51 | N | |
| ATOM | 10415 | C | LYS | C | 356 | 35.105 | 19.387 | 6.043 | 1.00 | 73.73 | C | |
| ATOM | 10416 | O | LYS | C | 356 | 35.679 | 20.430 | 5.728 | 1.00 | 73.92 | O | |
| ATOM | 10418 | N | LYS | C | 357 | 35.204 | 18.244 | 5.344 | 1.00 | 73.81 | N | |
| ATOM | 10419 | CA | LYS | C | 357 | 36.031 | 18.095 | 4.109 | 1.00 | 73.75 | C | |
| ATOM | 10421 | CB | LYS | C | 357 | 36.407 | 16.609 | 3.882 | 1.00 | 73.68 | C | |
| ATOM | 10424 | CG | LYS | C | 357 | 37.355 | 15.991 | 4.901 | 1.00 | 73.75 | C | |
| ATOM | 10427 | CD | LYS | C | 357 | 38.412 | 15.095 | 4.208 | 1.00 | 75.02 | C | |
| ATOM | 10430 | CE | LYS | C | 357 | 39.583 | 15.925 | 3.544 | 1.00 | 75.77 | C | |
| ATOM | 10433 | NZ | LYS | C | 357 | 40.144 | 15.386 | 2.221 | 1.00 | 74.30 | N | |
| ATOM | 10437 | C | LYS | C | 357 | 35.431 | 18.676 | 2.785 | 1.00 | 73.60 | C | |
| ATOM | 10438 | O | LYS | C | 357 | 36.046 | 18.553 | 1.718 | 1.00 | 73.29 | O | |
| ATOM | 10440 | N | ASN | C | 358 | 34.262 | 19.309 | 2.859 | 1.00 | 73.62 | N | |
| ATOM | 10441 | CA | ASN | C | 358 | 33.518 | 19.796 | 1.689 | 1.00 | 73.61 | C | |
| ATOM | 10443 | CB | ASN | C | 358 | 34.198 | 21.059 | 1.114 | 1.00 | 73.51 | C | |
| ATOM | 10446 | CG | ASN | C | 358 | 33.189 | 22.118 | 0.630 | 1.00 | 73.98 | C | |
| ATOM | 10447 | OD1 | ASN | C | 358 | 32.285 | 22.523 | 1.366 | 1.00 | 73.68 | O | |
| ATOM | 10448 | ND2 | ASN | C | 358 | 33.361 | 22.583 | −0.606 | 1.00 | 74.99 | N | |
| ATOM | 10451 | C | ASN | C | 358 | 33.223 | 18.711 | 0.622 | 1.00 | 73.66 | C | |
| ATOM | 10452 | O | ASN | C | 358 | 33.336 | 18.966 | −0.576 | 1.00 | 73.82 | O | |
| ATOM | 10454 | N | PHE | C | 359 | 32.846 | 17.507 | 1.097 | 1.00 | 73.84 | N | |
| ATOM | 10455 | CA | PHE | C | 359 | 32.330 | 16.385 | 0.269 | 1.00 | 73.50 | C | |
| ATOM | 10457 | CB | PHE | C | 359 | 32.934 | 15.039 | 0.691 | 1.00 | 74.08 | C | |
| ATOM | 10460 | CG | PHE | C | 359 | 34.325 | 14.844 | 0.215 | 1.00 | 74.97 | C | |
| ATOM | 10461 | CD1 | PHE | C | 359 | 35.358 | 15.531 | 0.823 | 1.00 | 76.55 | C | |
| ATOM | 10463 | CE1 | PHE | C | 359 | 36.685 | 15.383 | 0.390 | 1.00 | 76.77 | C | |
| ATOM | 10465 | CZ | PHE | C | 359 | 36.975 | 14.541 | −0.691 | 1.00 | 76.55 | C | |
| ATOM | 10467 | CE2 | PHE | C | 359 | 35.942 | 13.847 | −1.317 | 1.00 | 77.48 | C | |
| ATOM | 10469 | CD2 | PHE | C | 359 | 34.616 | 13.997 | −0.855 | 1.00 | 77.20 | C | |
| ATOM | 10471 | C | PHE | C | 359 | 30.816 | 16.241 | 0.371 | 1.00 | 72.79 | C | |
| ATOM | 10472 | O | PHE | C | 359 | 30.250 | 16.539 | 1.430 | 1.00 | 73.12 | O | |
| ATOM | 10474 | N | ILE | C | 360 | 30.191 | 15.789 | −0.733 | 1.00 | 71.60 | N | |
| ATOM | 10475 | CA | ILE | C | 360 | 28.814 | 15.278 | −0.781 | 1.00 | 70.28 | C | |
| ATOM | 10477 | CB | ILE | C | 360 | 28.011 | 15.872 | −1.958 | 1.00 | 70.65 | C | |
| ATOM | 10479 | CG1 | ILE | C | 360 | 28.187 | 17.361 | −2.091 | 1.00 | 70.74 | C | |
| ATOM | 10482 | CD1 | ILE | C | 360 | 27.859 | 18.118 | −0.801 | 1.00 | 71.56 | C | |
| ATOM | 10486 | CG2 | ILE | C | 360 | 26.498 | 15.586 | −1.750 | 1.00 | 73.25 | C | |
| ATOM | 10490 | C | ILE | C | 360 | 28.853 | 13.782 | −1.011 | 1.00 | 68.87 | C | |
| ATOM | 10491 | O | ILE | C | 360 | 29.685 | 13.310 | −1.769 | 1.00 | 68.78 | O | |
| ATOM | 10493 | N | HIS | C | 361 | 27.920 | 13.052 | −0.412 | 1.00 | 67.13 | N | |
| ATOM | 10494 | CA | HIS | C | 361 | 27.726 | 11.631 | −0.711 | 1.00 | 65.94 | C | |
| ATOM | 10496 | CB | HIS | C | 361 | 27.259 | 10.873 | 0.529 | 1.00 | 65.39 | C | |
| ATOM | 10499 | CG | HIS | C | 361 | 27.172 | 9.399 | 0.342 | 1.00 | 62.87 | C | |
| ATOM | 10500 | ND1 | HIS | C | 361 | 26.174 | 8.802 | −0.393 | 1.00 | 62.74 | N | |
| ATOM | 10502 | CE1 | HIS | C | 361 | 26.344 | 7.497 | −0.396 | 1.00 | 62.57 | C | |
| ATOM | 10504 | NE2 | HIS | C | 361 | 27.410 | 7.225 | 0.328 | 1.00 | 62.24 | N | |
| ATOM | 10506 | CD2 | HIS | C | 361 | 27.952 | 8.400 | 0.792 | 1.00 | 61.19 | C | |
| ATOM | 10508 | C | HIS | C | 361 | 26.725 | 11.419 | −1.832 | 1.00 | 65.91 | C | |
| ATOM | 10509 | O | HIS | C | 361 | 26.912 | 10.526 | −2.648 | 1.00 | 65.95 | O | |
| ATOM | 10511 | N | ARG | C | 362 | 25.610 | 12.151 | −1.799 | 1.00 | 65.73 | N | |
| ATOM | 10512 | CA | ARG | C | 362 | 24.551 | 12.109 | −2.852 | 1.00 | 65.41 | C | |
| ATOM | 10514 | CB | ARG | C | 362 | 25.102 | 12.519 | −4.242 | 1.00 | 65.29 | C | |
| ATOM | 10517 | CG | ARG | C | 362 | 25.276 | 14.025 | −4.434 | 1.00 | 64.75 | C | |
| ATOM | 10520 | CD | ARG | C | 362 | 25.170 | 14.478 | −5.830 | 1.00 | 64.51 | C | |
| ATOM | 10523 | NE | ARG | C | 362 | 26.181 | 13.800 | −6.623 | 1.00 | 63.60 | N | |
| ATOM | 10525 | CZ | ARG | C | 362 | 26.090 | 13.564 | −7.919 | 1.00 | 65.57 | C | |
| ATOM | 10526 | NH1 | ARG | C | 362 | 25.044 | 13.989 | −8.636 | 1.00 | 65.63 | N | |
| ATOM | 10529 | NH2 | ARG | C | 362 | 27.079 | 12.909 | −8.529 | 1.00 | 66.33 | N | |
| ATOM | 10532 | C | ARG | C | 362 | 23.716 | 10.841 | −2.982 | 1.00 | 65.05 | C | |
| ATOM | 10533 | O | ARG | C | 362 | 22.864 | 10.770 | −3.837 | 1.00 | 64.64 | O | |
| ATOM | 10535 | N | ASP | C | 363 | 23.916 | 9.859 | −2.122 | 1.00 | 66.16 | N | |
| ATOM | 10536 | CA | ASP | C | 363 | 23.138 | 8.580 | −2.178 | 1.00 | 66.80 | C | |
| ATOM | 10538 | CB | ASP | C | 363 | 23.724 | 7.584 | −3.209 | 1.00 | 67.10 | C | |
| ATOM | 10541 | CG | ASP | C | 363 | 22.657 | 6.736 | −3.891 | 1.00 | 67.76 | C | |
| ATOM | 10542 | OD1 | ASP | C | 363 | 21.472 | 7.119 | −3.971 | 1.00 | 69.17 | O | |
| ATOM | 10543 | OD2 | ASP | C | 363 | 22.937 | 5.648 | −4.402 | 1.00 | 73.12 | O | |
| ATOM | 10544 | C | ASP | C | 363 | 22.960 | 7.926 | −0.825 | 1.00 | 66.80 | C | |
| ATOM | 10545 | O | ASP | C | 363 | 22.964 | 6.712 | −0.691 | 1.00 | 67.07 | O | |
| ATOM | 10547 | N | LEU | C | 364 | 22.700 | 8.779 | 0.150 | 1.00 | 67.11 | N | |
| ATOM | 10548 | CA | LEU | C | 364 | 22.548 | 8.401 | 1.528 | 1.00 | 67.33 | C | |
| ATOM | 10550 | CB | LEU | C | 364 | 22.754 | 9.631 | 2.383 | 1.00 | 67.25 | C | |
| ATOM | 10553 | CG | LEU | C | 364 | 22.844 | 9.456 | 3.874 | 1.00 | 68.89 | C | |
| ATOM | 10555 | CD1 | LEU | C | 364 | 24.041 | 8.596 | 4.243 | 1.00 | 69.80 | C | |
| ATOM | 10559 | CD2 | LEU | C | 364 | 22.929 | 10.869 | 4.506 | 1.00 | 68.61 | C | |
| ATOM | 10563 | C | LEU | C | 364 | 21.144 | 7.808 | 1.729 | 1.00 | 66.91 | C | |
| ATOM | 10564 | O | LEU | C | 364 | 20.124 | 8.484 | 1.554 | 1.00 | 67.30 | O | |
| ATOM | 10566 | N | ALA | C | 365 | 21.115 | 6.531 | 2.070 | 1.00 | 66.28 | N | |
| ATOM | 10567 | CA | ALA | C | 365 | 19.890 | 5.777 | 2.236 | 1.00 | 65.79 | C | |
| ATOM | 10569 | CB | ALA | C | 365 | 19.366 | 5.381 | 0.892 | 1.00 | 65.60 | C | |
| ATOM | 10573 | C | ALA | C | 365 | 20.212 | 4.541 | 3.073 | 1.00 | 65.63 | C | |
| ATOM | 10574 | O | ALA | C | 365 | 21.365 | 4.140 | 3.175 | 1.00 | 65.09 | O | |
| ATOM | 10576 | N | ALA | C | 366 | 19.191 | 3.955 | 3.679 | 1.00 | 66.17 | N | |
| ATOM | 10577 | CA | ALA | C | 366 | 19.353 | 2.743 | 4.498 | 1.00 | 66.60 | C | |
| ATOM | 10579 | CB | ALA | C | 366 | 18.080 | 2.387 | 5.214 | 1.00 | 66.17 | C | |
| ATOM | 10583 | C | ALA | C | 366 | 19.820 | 1.582 | 3.629 | 1.00 | 67.39 | C | |
| ATOM | 10584 | O | ALA | C | 366 | 20.728 | 0.864 | 4.006 | 1.00 | 66.90 | O | |
| ATOM | 10586 | N | ARG | C | 367 | 19.265 | 1.461 | 2.429 | 1.00 | 68.85 | N | |
| ATOM | 10587 | CA | ARG | C | 367 | 19.756 | 0.458 | 1.457 | 1.00 | 70.33 | C | |
| ATOM | 10589 | CB | ARG | C | 367 | 18.864 | 0.397 | 0.178 | 1.00 | 70.25 | C | |
| ATOM | 10592 | CG | ARG | C | 367 | 19.265 | 1.360 | −0.917 | 1.00 | 70.79 | C | |
| ATOM | 10595 | CD | ARG | C | 367 | 18.181 | 1.725 | −1.920 | 1.00 | 71.07 | C | |
| ATOM | 10598 | NE | ARG | C | 367 | 18.608 | 2.979 | −2.535 | 1.00 | 70.84 | N | |
| ATOM | 10600 | CZ | ARG | C | 367 | 18.080 | 4.191 | −2.371 | 1.00 | 70.63 | C | |
| ATOM | 10601 | NH1 | ARG | C | 367 | 16.980 | 4.418 | −1.668 | 1.00 | 70.61 | N | |
| ATOM | 10604 | NH2 | ARG | C | 367 | 18.660 | 5.223 | −2.988 | 1.00 | 73.40 | N | |
| ATOM | 10607 | C | ARG | C | 367 | 21.273 | 0.583 | 1.108 | 1.00 | 70.73 | C | |
| ATOM | 10608 | O | ARG | C | 367 | 21.873 | −0.421 | 0.677 | 1.00 | 70.95 | O | |
| ATOM | 10610 | N | ASN | C | 368 | 21.843 | 1.797 | 1.286 | 1.00 | 71.08 | N | |
| ATOM | 10611 | CA | ASN | C | 368 | 23.283 | 2.097 | 1.122 | 1.00 | 71.28 | C | |
| ATOM | 10613 | CB | ASN | C | 368 | 23.455 | 3.417 | 0.334 | 1.00 | 71.74 | C | |
| ATOM | 10616 | CG | ASN | C | 368 | 23.137 | 3.275 | −1.149 | 1.00 | 73.25 | C | |
| ATOM | 10617 | OD1 | ASN | C | 368 | 23.290 | 2.201 | −1.746 | 1.00 | 75.56 | O | |
| ATOM | 10618 | ND2 | ASN | C | 368 | 22.707 | 4.375 | −1.760 | 1.00 | 74.86 | N | |
| ATOM | 10621 | C | ASN | C | 368 | 24.130 | 2.159 | 2.408 | 1.00 | 71.83 | C | |
| ATOM | 10622 | O | ASN | C | 368 | 25.313 | 2.532 | 2.356 | 1.00 | 71.11 | O | |
| ATOM | 10624 | N | CYS | C | 369 | 23.562 | 1.766 | 3.549 | 1.00 | 72.84 | N | |
| ATOM | 10625 | CA | CYS | C | 369 | 24.367 | 1.535 | 4.759 | 1.00 | 73.90 | C | |
| ATOM | 10627 | CB | CYS | C | 369 | 23.640 | 2.028 | 5.973 | 1.00 | 74.12 | C | |
| ATOM | 10630 | SG | CYS | C | 369 | 23.632 | 3.814 | 5.925 | 1.00 | 78.42 | S | |
| ATOM | 10632 | C | CYS | C | 369 | 24.843 | 0.088 | 4.969 | 1.00 | 74.14 | C | |
| ATOM | 10633 | O | CYS | C | 369 | 24.259 | −0.877 | 4.468 | 1.00 | 73.66 | O | |
| ATOM | 10635 | N | LEU | C | 370 | 25.943 | −0.022 | 5.706 | 1.00 | 74.47 | N | |
| ATOM | 10636 | CA | LEU | C | 370 | 26.578 | −1.286 | 5.976 | 1.00 | 74.69 | C | |
| ATOM | 10638 | CB | LEU | C | 370 | 27.912 | −1.380 | 5.235 | 1.00 | 74.94 | C | |
| ATOM | 10641 | CG | LEU | C | 370 | 27.828 | −1.627 | 3.713 | 1.00 | 74.82 | C | |
| ATOM | 10643 | CD1 | LEU | C | 370 | 29.253 | −1.663 | 3.119 | 1.00 | 75.15 | C | |
| ATOM | 10647 | CD2 | LEU | C | 370 | 27.085 | −2.897 | 3.325 | 1.00 | 74.34 | C | |
| ATOM | 10651 | C | LEU | C | 370 | 26.744 | −1.451 | 7.471 | 1.00 | 74.91 | C | |
| ATOM | 10652 | O | LEU | C | 370 | 27.175 | −0.515 | 8.155 | 1.00 | 74.32 | O | |
| ATOM | 10654 | N | VAL | C | 371 | 26.395 | −2.658 | 7.944 | 1.00 | 75.51 | N | |
| ATOM | 10655 | CA | VAL | C | 371 | 26.285 | −3.003 | 9.358 | 1.00 | 75.78 | C | |
| ATOM | 10657 | CB | VAL | C | 371 | 24.894 | −3.598 | 9.672 | 1.00 | 75.87 | C | |
| ATOM | 10659 | CG1 | VAL | C | 371 | 24.737 | −3.873 | 11.190 | 1.00 | 76.53 | C | |
| ATOM | 10663 | CG2 | VAL | C | 371 | 23.761 | −2.682 | 9.164 | 1.00 | 76.14 | C | |
| ATOM | 10667 | C | VAL | C | 371 | 27.345 | −4.052 | 9.668 | 1.00 | 76.10 | C | |
| ATOM | 10668 | O | VAL | C | 371 | 27.380 | −5.104 | 9.017 | 1.00 | 75.96 | O | |
| ATOM | 10670 | N | GLY | C | 372 | 28.216 | −3.740 | 10.628 | 1.00 | 76.55 | N | |
| ATOM | 10671 | CA | GLY | C | 372 | 29.107 | −4.719 | 11.235 | 1.00 | 77.27 | C | |
| ATOM | 10674 | C | GLY | C | 372 | 28.423 | −5.477 | 12.380 | 1.00 | 77.98 | C | |
| ATOM | 10675 | O | GLY | C | 372 | 27.396 | −5.025 | 12.914 | 1.00 | 78.29 | O | |
| ATOM | 10677 | N | GLU | C | 373 | 28.999 | −6.620 | 12.773 | 1.00 | 78.31 | N | |
| ATOM | 10678 | CA | GLU | C | 373 | 28.630 | −7.290 | 14.025 | 1.00 | 78.90 | C | |
| ATOM | 10680 | CB | GLU | C | 373 | 29.574 | −8.460 | 14.335 | 1.00 | 79.37 | C | |
| ATOM | 10683 | CG | GLU | C | 373 | 29.536 | −9.627 | 13.339 | 1.00 | 80.54 | C | |
| ATOM | 10686 | CD | GLU | C | 373 | 28.849 | −10.904 | 13.837 | 1.00 | 81.77 | C | |
| ATOM | 10687 | OE1 | GLU | C | 373 | 28.742 | −11.152 | 15.063 | 1.00 | 81.02 | O | |
| ATOM | 10688 | OE2 | GLU | C | 373 | 28.440 | −11.707 | 12.965 | 1.00 | 83.70 | O | |
| ATOM | 10689 | C | GLU | C | 373 | 28.703 | −6.273 | 15.180 | 1.00 | 79.06 | C | |
| ATOM | 10690 | O | GLU | C | 373 | 29.473 | −5.293 | 15.102 | 1.00 | 79.11 | O | |
| ATOM | 10692 | N | ASN | C | 374 | 27.887 | −6.499 | 16.218 | 1.00 | 78.78 | N | |
| ATOM | 10693 | CA | ASN | C | 374 | 27.778 | −5.593 | 17.376 | 1.00 | 78.46 | C | |
| ATOM | 10695 | CB | ASN | C | 374 | 29.088 | −5.614 | 18.201 | 1.00 | 78.69 | C | |
| ATOM | 10698 | CG | ASN | C | 374 | 29.434 | −7.000 | 18.745 | 1.00 | 79.41 | C | |
| ATOM | 10699 | OD1 | ASN | C | 374 | 28.619 | −7.924 | 18.715 | 1.00 | 80.93 | O | |
| ATOM | 10700 | ND2 | ASN | C | 374 | 30.638 | −7.131 | 19.289 | 1.00 | 80.02 | N | |
| ATOM | 10703 | C | ASN | C | 374 | 27.375 | −4.158 | 17.009 | 1.00 | 77.73 | C | |
| ATOM | 10704 | O | ASN | C | 374 | 27.845 | −3.181 | 17.608 | 1.00 | 77.21 | O | |
| ATOM | 10706 | N | HIS | C | 375 | 26.537 | −4.045 | 15.981 | 1.00 | 77.30 | N | |
| ATOM | 10707 | CA | HIS | C | 375 | 25.909 | −2.782 | 15.588 | 1.00 | 77.05 | C | |
| ATOM | 10709 | CB | HIS | C | 375 | 24.803 | −2.437 | 16.595 | 1.00 | 77.01 | C | |
| ATOM | 10712 | CG | HIS | C | 375 | 23.865 | −3.576 | 16.838 | 1.00 | 77.05 | C | |
| ATOM | 10713 | ND1 | HIS | C | 375 | 23.476 | −3.971 | 18.099 | 1.00 | 76.76 | N | |
| ATOM | 10715 | CE1 | HIS | C | 375 | 22.676 | −5.019 | 18.003 | 1.00 | 77.09 | C | |
| ATOM | 10717 | NE2 | HIS | C | 375 | 22.547 | −5.328 | 16.725 | 1.00 | 77.39 | N | |
| ATOM | 10719 | CD2 | HIS | C | 375 | 23.291 | −4.449 | 15.976 | 1.00 | 77.16 | C | |
| ATOM | 10721 | C | HIS | C | 375 | 26.854 | −1.592 | 15.335 | 1.00 | 76.87 | C | |
| ATOM | 10722 | O | HIS | C | 375 | 26.455 | −0.434 | 15.512 | 1.00 | 77.07 | O | |
| ATOM | 10724 | N | LEU | C | 376 | 28.089 | −1.881 | 14.889 | 1.00 | 76.49 | N | |
| ATOM | 10725 | CA | LEU | C | 376 | 28.924 | −0.871 | 14.198 | 1.00 | 75.58 | C | |
| ATOM | 10727 | CB | LEU | C | 376 | 30.389 | −1.317 | 14.033 | 1.00 | 75.43 | C | |
| ATOM | 10730 | CG | LEU | C | 376 | 31.433 | −0.190 | 14.069 | 1.00 | 75.58 | C | |
| ATOM | 10732 | CD1 | LEU | C | 376 | 32.865 | −0.754 | 14.175 | 1.00 | 75.63 | C | |
| ATOM | 10736 | CD2 | LEU | C | 376 | 31.320 | 0.730 | 12.864 | 1.00 | 75.97 | C | |
| ATOM | 10740 | C | LEU | C | 376 | 28.247 | −0.622 | 12.842 | 1.00 | 75.01 | C | |
| ATOM | 10741 | O | LEU | C | 376 | 27.794 | −1.570 | 12.197 | 1.00 | 74.60 | O | |
| ATOM | 10743 | N | VAL | C | 377 | 28.130 | 0.645 | 12.450 | 1.00 | 74.55 | N | |
| ATOM | 10744 | CA | VAL | C | 377 | 27.545 | 1.036 | 11.149 | 1.00 | 74.53 | C | |
| ATOM | 10746 | CB | VAL | C | 377 | 26.158 | 1.697 | 11.350 | 1.00 | 74.35 | C | |
| ATOM | 10748 | CG1 | VAL | C | 377 | 25.383 | 1.752 | 10.052 | 1.00 | 74.07 | C | |
| ATOM | 10752 | CG2 | VAL | C | 377 | 25.353 | 0.911 | 12.363 | 1.00 | 74.99 | C | |
| ATOM | 10756 | C | VAL | C | 377 | 28.491 | 1.980 | 10.366 | 1.00 | 74.29 | C | |
| ATOM | 10757 | O | VAL | C | 377 | 29.067 | 2.905 | 10.957 | 1.00 | 74.83 | O | |
| ATOM | 10759 | N | LYS | C | 378 | 28.673 | 1.728 | 9.061 | 1.00 | 73.71 | N | |
| ATOM | 10760 | CA | LYS | C | 378 | 29.444 | 2.635 | 8.164 | 1.00 | 73.37 | C | |
| ATOM | 10762 | CB | LYS | C | 378 | 30.814 | 2.062 | 7.770 | 1.00 | 72.70 | C | |
| ATOM | 10765 | CG | LYS | C | 378 | 31.599 | 1.521 | 8.945 | 1.00 | 72.86 | C | |
| ATOM | 10768 | CD | LYS | C | 378 | 33.097 | 1.644 | 8.812 | 1.00 | 72.42 | C | |
| ATOM | 10771 | CE | LYS | C | 378 | 33.810 | 0.793 | 9.861 | 1.00 | 71.56 | C | |
| ATOM | 10774 | NZ | LYS | C | 378 | 35.156 | 1.343 | 10.150 | 1.00 | 71.67 | N | |
| ATOM | 10778 | C | LYS | C | 378 | 28.661 | 2.974 | 6.903 | 1.00 | 73.28 | C | |
| ATOM | 10779 | O | LYS | C | 378 | 27.950 | 2.130 | 6.340 | 1.00 | 73.06 | O | |
| ATOM | 10781 | N | VAL | C | 379 | 28.817 | 4.218 | 6.454 | 1.00 | 73.58 | N | |
| ATOM | 10782 | CA | VAL | C | 379 | 28.185 | 4.692 | 5.226 | 1.00 | 73.90 | C | |
| ATOM | 10784 | CB | VAL | C | 379 | 28.190 | 6.240 | 5.116 | 1.00 | 73.92 | C | |
| ATOM | 10786 | CG1 | VAL | C | 379 | 27.599 | 6.705 | 3.767 | 1.00 | 73.30 | C | |
| ATOM | 10790 | CG2 | VAL | C | 379 | 27.419 | 6.856 | 6.303 | 1.00 | 74.15 | C | |
| ATOM | 10794 | C | VAL | C | 379 | 28.969 | 4.077 | 4.070 | 1.00 | 74.27 | C | |
| ATOM | 10795 | O | VAL | C | 379 | 30.213 | 4.164 | 4.048 | 1.00 | 74.38 | O | |
| ATOM | 10797 | N | ALA | C | 380 | 28.237 | 3.449 | 3.144 | 1.00 | 74.28 | N | |
| ATOM | 10798 | CA | ALA | C | 380 | 28.809 | 2.844 | 1.946 | 1.00 | 74.06 | C | |
| ATOM | 10800 | CB | ALA | C | 380 | 28.313 | 1.399 | 1.793 | 1.00 | 73.97 | C | |
| ATOM | 10804 | C | ALA | C | 380 | 28.498 | 3.659 | 0.680 | 1.00 | 73.73 | C | |
| ATOM | 10805 | O | ALA | C | 380 | 27.893 | 4.728 | 0.733 | 1.00 | 73.31 | O | |
| ATOM | 10807 | N | ASP | C | 381 | 29.020 | 3.135 | −0.429 | 1.00 | 73.47 | N | |
| ATOM | 10808 | CA | ASP | C | 381 | 28.584 | 3.382 | −1.796 | 1.00 | 72.91 | C | |
| ATOM | 10810 | CB | ASP | C | 381 | 27.106 | 3.040 | −2.002 | 1.00 | 73.22 | C | |
| ATOM | 10813 | CG | ASP | C | 381 | 26.776 | 2.794 | −3.467 | 1.00 | 74.76 | C | |
| ATOM | 10814 | OD1 | ASP | C | 381 | 27.282 | 3.542 | −4.354 | 1.00 | 75.68 | O | |
| ATOM | 10815 | OD2 | ASP | C | 381 | 26.040 | 1.839 | −3.818 | 1.00 | 77.52 | O | |
| ATOM | 10816 | C | ASP | C | 381 | 28.879 | 4.766 | −2.232 | 1.00 | 72.08 | C | |
| ATOM | 10817 | O | ASP | C | 381 | 27.959 | 5.598 | −2.326 | 1.00 | 72.39 | O | |
| ATOM | 10819 | N | PHE | C | 382 | 30.156 | 5.004 | −2.534 | 1.00 | 71.02 | N | |
| ATOM | 10820 | CA | PHE | C | 382 | 30.617 | 6.353 | −2.851 | 1.00 | 70.16 | C | |
| ATOM | 10822 | CB | PHE | C | 382 | 31.970 | 6.601 | −2.205 | 1.00 | 69.45 | C | |
| ATOM | 10825 | CG | PHE | C | 382 | 31.903 | 6.582 | −0.712 | 1.00 | 68.96 | C | |
| ATOM | 10826 | CD1 | PHE | C | 382 | 31.587 | 7.743 | 0.000 | 1.00 | 68.39 | C | |
| ATOM | 10828 | CE1 | PHE | C | 382 | 31.500 | 7.723 | 1.385 | 1.00 | 67.82 | C | |
| ATOM | 10830 | CZ | PHE | C | 382 | 31.705 | 6.530 | 2.066 | 1.00 | 68.20 | C | |
| ATOM | 10832 | CE2 | PHE | C | 382 | 32.001 | 5.363 | 1.359 | 1.00 | 67.57 | C | |
| ATOM | 10834 | CD2 | PHE | C | 382 | 32.080 | 5.392 | −0.008 | 1.00 | 68.35 | C | |
| ATOM | 10836 | C | PHE | C | 382 | 30.559 | 6.713 | −4.344 | 1.00 | 70.25 | C | |
| ATOM | 10837 | O | PHE | C | 382 | 30.979 | 7.799 | −4.717 | 1.00 | 70.36 | O | |
| ATOM | 10839 | N | GLY | C | 383 | 29.954 | 5.846 | −5.169 | 1.00 | 70.23 | N | |
| ATOM | 10840 | CA | GLY | C | 383 | 29.744 | 6.062 | −6.607 | 1.00 | 70.11 | C | |
| ATOM | 10843 | C | GLY | C | 383 | 29.261 | 7.402 | −7.128 | 1.00 | 70.15 | C | |
| ATOM | 10844 | O | GLY | C | 383 | 29.461 | 7.684 | −8.312 | 1.00 | 70.61 | O | |
| ATOM | 10846 | N | LEU | C | 384 | 28.597 | 8.197 | −6.281 | 1.00 | 69.98 | N | |
| ATOM | 10847 | CA | LEU | C | 384 | 28.139 | 9.559 | −6.625 | 1.00 | 69.89 | C | |
| ATOM | 10849 | CB | LEU | C | 384 | 26.610 | 9.651 | −6.474 | 1.00 | 69.79 | C | |
| ATOM | 10852 | CG | LEU | C | 384 | 25.830 | 8.862 | −7.506 | 1.00 | 69.31 | C | |
| ATOM | 10854 | CD1 | LEU | C | 384 | 24.400 | 8.774 | −7.093 | 1.00 | 69.27 | C | |
| ATOM | 10858 | CD2 | LEU | C | 384 | 25.970 | 9.511 | −8.880 | 1.00 | 70.35 | C | |
| ATOM | 10862 | C | LEU | C | 384 | 28.762 | 10.639 | −5.777 | 1.00 | 69.86 | C | |
| ATOM | 10863 | O | LEU | C | 384 | 28.367 | 11.803 | −5.878 | 1.00 | 69.99 | O | |
| ATOM | 10865 | N | SER | C | 385 | 29.735 | 10.278 | −4.953 | 1.00 | 69.52 | N | |
| ATOM | 10866 | CA | SER | C | 385 | 30.379 | 11.250 | −4.087 | 1.00 | 69.55 | C | |
| ATOM | 10868 | CB | SER | C | 385 | 31.167 | 10.544 | −3.006 | 1.00 | 69.26 | C | |
| ATOM | 10871 | OG | SER | C | 385 | 30.332 | 9.564 | −2.437 | 1.00 | 69.75 | O | |
| ATOM | 10873 | C | SER | C | 385 | 31.267 | 12.203 | −4.879 | 1.00 | 69.52 | C | |
| ATOM | 10874 | O | SER | C | 385 | 31.755 | 11.854 | −5.930 | 1.00 | 68.74 | O | |
| ATOM | 10876 | N | ARG | C | 386 | 31.398 | 13.426 | −4.379 | 1.00 | 70.34 | N | |
| ATOM | 10877 | CA | ARG | C | 386 | 32.196 | 14.475 | −4.999 | 1.00 | 71.15 | C | |
| ATOM | 10879 | CB | ARG | C | 386 | 31.304 | 15.433 | −5.794 | 1.00 | 71.46 | C | |
| ATOM | 10882 | CG | ARG | C | 386 | 30.439 | 14.805 | −6.875 | 1.00 | 72.73 | C | |
| ATOM | 10885 | CD | ARG | C | 386 | 31.220 | 14.228 | −8.046 | 1.00 | 74.32 | C | |
| ATOM | 10888 | NE | ARG | C | 386 | 30.539 | 13.065 | −8.607 | 1.00 | 74.51 | N | |
| ATOM | 10890 | CZ | ARG | C | 386 | 30.572 | 12.689 | −9.876 | 1.00 | 76.36 | C | |
| ATOM | 10891 | NH1 | ARG | C | 386 | 31.252 | 13.366 | −10.800 | 1.00 | 79.35 | N | |
| ATOM | 10894 | NH2 | ARG | C | 386 | 29.883 | 11.616 | −10.248 | 1.00 | 77.73 | N | |
| ATOM | 10897 | C | ARG | C | 386 | 32.856 | 15.296 | −3.927 | 1.00 | 71.72 | C | |
| ATOM | 10898 | O | ARG | C | 386 | 32.207 | 15.587 | −2.945 | 1.00 | 71.34 | O | |
| ATOM | 10900 | N | LEU | C | 387 | 34.124 | 15.681 | −4.128 | 1.00 | 73.15 | N | |
| ATOM | 10901 | CA | LEU | C | 387 | 34.720 | 16.828 | −3.416 | 1.00 | 73.54 | C | |
| ATOM | 10903 | CB | LEU | C | 387 | 36.251 | 16.863 | −3.510 | 1.00 | 73.60 | C | |
| ATOM | 10906 | CG | LEU | C | 387 | 36.900 | 18.178 | −2.998 | 1.00 | 73.83 | C | |
| ATOM | 10908 | CD1 | LEU | C | 387 | 36.768 | 18.339 | −1.476 | 1.00 | 74.41 | C | |
| ATOM | 10912 | CD2 | LEU | C | 387 | 38.360 | 18.305 | −3.433 | 1.00 | 73.71 | C | |
| ATOM | 10916 | C | LEU | C | 387 | 34.174 | 18.079 | −4.056 | 1.00 | 73.78 | C | |
| ATOM | 10917 | O | LEU | C | 387 | 34.561 | 18.398 | −5.170 | 1.00 | 74.01 | O | |
| ATOM | 10919 | N | MET | C | 388 | 33.302 | 18.794 | −3.361 | 1.00 | 74.42 | N | |
| ATOM | 10920 | CA | MET | C | 388 | 32.767 | 20.044 | −3.905 | 1.00 | 75.15 | C | |
| ATOM | 10922 | CB | MET | C | 388 | 31.679 | 20.612 | −3.017 | 1.00 | 75.17 | C | |
| ATOM | 10925 | CG | MET | C | 388 | 30.451 | 19.808 | −2.933 | 1.00 | 74.40 | C | |
| ATOM | 10928 | SD | MET | C | 388 | 29.102 | 20.928 | −2.553 | 1.00 | 75.17 | S | |
| ATOM | 10929 | CE | MET | C | 388 | 29.417 | 21.270 | −0.715 | 1.00 | 74.55 | C | |
| ATOM | 10933 | C | MET | C | 388 | 33.827 | 21.135 | −4.025 | 1.00 | 75.66 | C | |
| ATOM | 10934 | O | MET | C | 388 | 34.751 | 21.177 | −3.219 | 1.00 | 75.82 | O | |
| ATOM | 10936 | N | THR | C | 389 | 33.654 | 22.017 | −5.011 | 1.00 | 76.25 | N | |
| ATOM | 10937 | CA | THR | C | 389 | 34.418 | 23.268 | −5.117 | 1.00 | 76.88 | C | |
| ATOM | 10939 | CB | THR | C | 389 | 35.008 | 23.414 | −6.535 | 1.00 | 76.76 | C | |
| ATOM | 10941 | OG1 | THR | C | 389 | 35.764 | 22.243 | −6.875 | 1.00 | 76.73 | O | |
| ATOM | 10943 | CG2 | THR | C | 389 | 36.018 | 24.535 | −6.586 | 1.00 | 76.81 | C | |
| ATOM | 10947 | C | THR | C | 389 | 33.472 | 24.416 | −4.775 | 1.00 | 77.50 | C | |
| ATOM | 10948 | O | THR | C | 389 | 33.001 | 25.140 | −5.655 | 1.00 | 77.75 | O | |
| ATOM | 10950 | N | GLY | C | 390 | 33.184 | 24.555 | −3.483 | 1.00 | 78.25 | N | |
| ATOM | 10951 | CA | GLY | C | 390 | 32.197 | 25.517 | −2.975 | 1.00 | 78.69 | C | |
| ATOM | 10954 | C | GLY | C | 390 | 30.979 | 24.790 | −2.434 | 1.00 | 79.20 | C | |
| ATOM | 10955 | O | GLY | C | 390 | 31.119 | 23.718 | −1.829 | 1.00 | 79.08 | O | |
| ATOM | 10957 | N | ASP | C | 391 | 29.789 | 25.348 | −2.697 | 1.00 | 79.60 | N | |
| ATOM | 10958 | CA | ASP | C | 391 | 28.526 | 24.998 | −1.983 | 1.00 | 79.61 | C | |
| ATOM | 10960 | CB | ASP | C | 391 | 28.034 | 26.214 | −1.135 | 1.00 | 80.08 | C | |
| ATOM | 10963 | CG | ASP | C | 391 | 27.940 | 27.537 | −1.942 | 1.00 | 82.30 | C | |
| ATOM | 10964 | OD1 | ASP | C | 391 | 27.487 | 27.497 | −3.131 | 1.00 | 85.80 | O | |
| ATOM | 10965 | OD2 | ASP | C | 391 | 28.318 | 28.658 | −1.465 | 1.00 | 83.20 | O | |
| ATOM | 10966 | C | ASP | C | 391 | 27.421 | 24.480 | −2.922 | 1.00 | 79.02 | C | |
| ATOM | 10967 | O | ASP | C | 391 | 26.227 | 24.621 | −2.623 | 1.00 | 79.50 | O | |
| ATOM | 10969 | N | THR | C | 392 | 27.819 | 23.860 | −4.034 | 1.00 | 77.94 | N | |
| ATOM | 10970 | CA | THR | C | 392 | 26.911 | 23.557 | −5.138 | 1.00 | 77.36 | C | |
| ATOM | 10972 | CB | THR | C | 392 | 26.555 | 24.870 | −5.872 | 1.00 | 77.79 | C | |
| ATOM | 10974 | OG1 | THR | C | 392 | 25.635 | 25.633 | −5.088 | 1.00 | 78.85 | O | |
| ATOM | 10976 | CG2 | THR | C | 392 | 25.765 | 24.645 | −7.176 | 1.00 | 79.48 | C | |
| ATOM | 10980 | C | THR | C | 392 | 27.638 | 22.630 | −6.083 | 1.00 | 76.29 | C | |
| ATOM | 10981 | O | THR | C | 392 | 28.693 | 23.010 | −6.589 | 1.00 | 76.52 | O | |
| ATOM | 10983 | N | TYR | C | 393 | 27.103 | 21.424 | −6.287 | 1.00 | 74.87 | N | |
| ATOM | 10984 | CA | TYR | C | 393 | 27.511 | 20.533 | −7.385 | 1.00 | 73.85 | C | |
| ATOM | 10986 | CB | TYR | C | 393 | 28.140 | 19.238 | −6.894 | 1.00 | 73.35 | C | |
| ATOM | 10989 | CG | TYR | C | 393 | 28.539 | 18.283 | −8.026 | 1.00 | 74.10 | C | |
| ATOM | 10990 | CD1 | TYR | C | 393 | 27.597 | 17.374 | −8.610 | 1.00 | 73.98 | C | |
| ATOM | 10992 | CE1 | TYR | C | 393 | 27.974 | 16.496 | −9.669 | 1.00 | 73.05 | C | |
| ATOM | 10994 | CZ | TYR | C | 393 | 29.308 | 16.514 | −10.140 | 1.00 | 72.95 | C | |
| ATOM | 10995 | OH | TYR | C | 393 | 29.723 | 15.674 | −11.135 | 1.00 | 72.05 | O | |
| ATOM | 10997 | CE2 | TYR | C | 393 | 30.252 | 17.386 | −9.578 | 1.00 | 73.02 | C | |
| ATOM | 10999 | CD2 | TYR | C | 393 | 29.865 | 18.267 | −8.523 | 1.00 | 73.85 | C | |
| ATOM | 11001 | C | TYR | C | 393 | 26.272 | 20.199 | −8.191 | 1.00 | 73.27 | C | |
| ATOM | 11002 | O | TYR | C | 393 | 25.302 | 19.655 | −7.643 | 1.00 | 73.14 | O | |
| ATOM | 11004 | N | THR | C | 394 | 26.352 | 20.452 | −9.498 | 1.00 | 72.58 | N | |
| ATOM | 11005 | CA | THR | C | 394 | 25.208 | 20.425 | −10.387 | 1.00 | 71.86 | C | |
| ATOM | 11007 | CB | THR | C | 394 | 25.176 | 21.689 | −11.259 | 1.00 | 71.93 | C | |
| ATOM | 11009 | OG1 | THR | C | 394 | 25.353 | 22.860 | −10.442 | 1.00 | 70.42 | O | |
| ATOM | 11011 | CG2 | THR | C | 394 | 23.787 | 21.853 | −11.943 | 1.00 | 72.24 | C | |
| ATOM | 11015 | C | THR | C | 394 | 25.329 | 19.195 | −11.248 | 1.00 | 71.33 | C | |
| ATOM | 11016 | O | THR | C | 394 | 26.324 | 19.027 | −11.978 | 1.00 | 71.13 | O | |
| ATOM | 11018 | N | ALA | C | 395 | 24.321 | 18.325 | −11.145 | 1.00 | 70.98 | N | |
| ATOM | 11019 | CA | ALA | C | 395 | 24.228 | 17.136 | −12.002 | 1.00 | 70.55 | C | |
| ATOM | 11021 | CB | ALA | C | 395 | 23.264 | 16.075 | −11.389 | 1.00 | 70.05 | C | |
| ATOM | 11025 | C | ALA | C | 395 | 23.782 | 17.571 | −13.408 | 1.00 | 69.91 | C | |
| ATOM | 11026 | O | ALA | C | 395 | 23.225 | 18.653 | −13.586 | 1.00 | 70.86 | O | |
| ATOM | 11028 | N | HIS | C | 396 | 24.078 | 16.746 | −14.401 | 1.00 | 69.20 | N | |
| ATOM | 11029 | CA | HIS | C | 396 | 23.505 | 16.847 | −15.752 | 1.00 | 68.85 | C | |
| ATOM | 11031 | CB | HIS | C | 396 | 24.065 | 15.686 | −16.596 | 1.00 | 68.90 | C | |
| ATOM | 11034 | CG | HIS | C | 396 | 23.862 | 15.805 | −18.069 | 1.00 | 67.91 | C | |
| ATOM | 11035 | ND1 | HIS | C | 396 | 24.410 | 16.820 | −18.808 | 1.00 | 69.95 | N | |
| ATOM | 11037 | CE1 | HIS | C | 396 | 24.127 | 16.636 | −20.084 | 1.00 | 69.30 | C | |
| ATOM | 11039 | NE2 | HIS | C | 396 | 23.412 | 15.536 | −20.200 | 1.00 | 68.35 | N | |
| ATOM | 11041 | CD2 | HIS | C | 396 | 23.276 | 14.972 | −18.959 | 1.00 | 68.13 | C | |
| ATOM | 11043 | C | HIS | C | 396 | 21.958 | 16.770 | −15.686 | 1.00 | 68.93 | C | |
| ATOM | 11044 | O | HIS | C | 396 | 21.388 | 16.187 | −14.745 | 1.00 | 68.51 | O | |
| ATOM | 11046 | N | ALA | C | 397 | 21.297 | 17.409 | −16.663 | 1.00 | 69.13 | N | |
| ATOM | 11047 | CA | ALA | C | 397 | 19.879 | 17.141 | −16.992 | 1.00 | 68.99 | C | |
| ATOM | 11049 | CB | ALA | C | 397 | 19.431 | 18.012 | −18.156 | 1.00 | 68.62 | C | |
| ATOM | 11053 | C | ALA | C | 397 | 19.805 | 15.681 | −17.393 | 1.00 | 69.26 | C | |
| ATOM | 11054 | O | ALA | C | 397 | 20.767 | 15.146 | −17.877 | 1.00 | 69.54 | O | |
| ATOM | 11056 | N | GLY | C | 398 | 18.706 | 14.990 | −17.167 | 1.00 | 69.84 | N | |
| ATOM | 11057 | CA | GLY | C | 398 | 18.670 | 13.539 | −17.489 | 1.00 | 70.32 | C | |
| ATOM | 11060 | C | GLY | C | 398 | 19.658 | 12.642 | −16.733 | 1.00 | 70.29 | C | |
| ATOM | 11061 | O | GLY | C | 398 | 20.011 | 11.569 | −17.204 | 1.00 | 70.14 | O | |
| ATOM | 11063 | N | ALA | C | 399 | 20.153 | 13.129 | −15.598 | 1.00 | 70.65 | N | |
| ATOM | 11064 | CA | ALA | C | 399 | 20.597 | 12.289 | −14.501 | 1.00 | 70.68 | C | |
| ATOM | 11066 | CB | ALA | C | 399 | 21.698 | 12.961 | −13.703 | 1.00 | 70.53 | C | |
| ATOM | 11070 | C | ALA | C | 399 | 19.316 | 12.190 | −13.704 | 1.00 | 70.69 | C | |
| ATOM | 11071 | O | ALA | C | 399 | 18.622 | 13.204 | −13.536 | 1.00 | 70.86 | O | |
| ATOM | 11073 | N | LYS | C | 400 | 18.987 | 10.974 | −13.280 | 1.00 | 70.64 | N | |
| ATOM | 11074 | CA | LYS | C | 400 | 17.739 | 10.662 | −12.605 | 1.00 | 70.72 | C | |
| ATOM | 11076 | CB | LYS | C | 400 | 16.939 | 9.709 | −13.484 | 1.00 | 70.39 | C | |
| ATOM | 11079 | CG | LYS | C | 400 | 16.344 | 10.387 | −14.742 | 1.00 | 69.61 | C | |
| ATOM | 11085 | C | LYS | C | 400 | 18.062 | 10.077 | −11.219 | 1.00 | 71.47 | C | |
| ATOM | 11086 | O | LYS | C | 400 | 19.159 | 9.522 | −11.011 | 1.00 | 72.39 | O | |
| ATOM | 11088 | N | PHE | C | 401 | 17.136 | 10.221 | −10.264 | 1.00 | 71.61 | N | |
| ATOM | 11089 | CA | PHE | C | 401 | 17.444 | 10.011 | −8.833 | 1.00 | 71.70 | C | |
| ATOM | 11091 | CB | PHE | C | 401 | 17.774 | 11.362 | −8.183 | 1.00 | 72.45 | C | |
| ATOM | 11094 | CG | PHE | C | 401 | 19.085 | 11.921 | −8.646 | 1.00 | 73.32 | C | |
| ATOM | 11095 | CD1 | PHE | C | 401 | 20.301 | 11.378 | −8.149 | 1.00 | 72.55 | C | |
| ATOM | 11097 | CE1 | PHE | C | 401 | 21.549 | 11.858 | −8.584 | 1.00 | 72.75 | C | |
| ATOM | 11099 | CZ | PHE | C | 401 | 21.592 | 12.874 | −9.566 | 1.00 | 73.50 | C | |
| ATOM | 11101 | CE2 | PHE | C | 401 | 20.367 | 13.422 | −10.090 | 1.00 | 74.20 | C | |
| ATOM | 11103 | CD2 | PHE | C | 401 | 19.123 | 12.929 | −9.633 | 1.00 | 73.60 | C | |
| ATOM | 11105 | C | PHE | C | 401 | 16.325 | 9.360 | −8.051 | 1.00 | 71.66 | C | |
| ATOM | 11106 | O | PHE | C | 401 | 15.184 | 9.375 | −8.540 | 1.00 | 71.81 | O | |
| ATOM | 11108 | N | PRO | C | 402 | 16.634 | 8.790 | −6.859 | 1.00 | 71.00 | N | |
| ATOM | 11109 | CA | PRO | C | 402 | 15.576 | 8.365 | −5.893 | 1.00 | 70.25 | C | |
| ATOM | 11111 | CB | PRO | C | 402 | 16.356 | 7.729 | −4.727 | 1.00 | 70.20 | C | |
| ATOM | 11114 | CG | PRO | C | 402 | 17.805 | 8.042 | −4.962 | 1.00 | 70.93 | C | |
| ATOM | 11117 | CD | PRO | C | 402 | 17.990 | 8.440 | −6.382 | 1.00 | 70.75 | C | |
| ATOM | 11120 | C | PRO | C | 402 | 14.765 | 9.564 | −5.421 | 1.00 | 69.78 | C | |
| ATOM | 11121 | O | PRO | C | 402 | 15.288 | 10.402 | −4.716 | 1.00 | 69.93 | O | |
| ATOM | 11122 | N | ILE | C | 403 | 13.516 | 9.646 | −5.864 | 1.00 | 69.19 | N | |
| ATOM | 11123 | CA | ILE | C | 403 | 12.581 | 10.755 | −5.554 | 1.00 | 68.73 | C | |
| ATOM | 11125 | CB | ILE | C | 403 | 11.183 | 10.428 | −6.224 | 1.00 | 68.84 | C | |
| ATOM | 11127 | CG1 | ILE | C | 403 | 11.235 | 10.562 | −7.753 | 1.00 | 68.58 | C | |
| ATOM | 11130 | CD1 | ILE | C | 403 | 11.256 | 11.996 | −8.261 | 1.00 | 69.05 | C | |
| ATOM | 11134 | CG2 | ILE | C | 403 | 10.016 | 11.267 | −5.635 | 1.00 | 69.41 | C | |
| ATOM | 11138 | C | ILE | C | 403 | 12.379 | 10.958 | −4.047 | 1.00 | 67.74 | C | |
| ATOM | 11139 | O | ILE | C | 403 | 12.536 | 12.075 | −3.516 | 1.00 | 67.49 | O | |
| ATOM | 11141 | N | LYS | C | 404 | 11.986 | 9.859 | −3.398 | 1.00 | 67.02 | N | |
| ATOM | 11142 | CA | LYS | C | 404 | 11.675 | 9.800 | −1.957 | 1.00 | 66.38 | C | |
| ATOM | 11144 | CB | LYS | C | 404 | 11.238 | 8.362 | −1.537 | 1.00 | 67.26 | C | |
| ATOM | 11147 | CG | LYS | C | 404 | 9.679 | 8.043 | −1.625 | 1.00 | 67.27 | C | |
| ATOM | 11150 | CD | LYS | C | 404 | 9.274 | 7.204 | −2.843 | 1.00 | 67.48 | C | |
| ATOM | 11153 | CE | LYS | C | 404 | 7.829 | 6.564 | −2.722 | 1.00 | 68.82 | C | |
| ATOM | 11156 | NZ | LYS | C | 404 | 6.592 | 7.470 | −2.719 | 1.00 | 67.65 | N | |
| ATOM | 11160 | C | LYS | C | 404 | 12.789 | 10.296 | −1.025 | 1.00 | 65.74 | C | |
| ATOM | 11161 | O | LYS | C | 404 | 12.488 | 10.806 | 0.040 | 1.00 | 65.72 | O | |
| ATOM | 11163 | N | TRP | C | 405 | 14.047 | 10.166 | −1.445 | 1.00 | 65.01 | N | |
| ATOM | 11164 | CA | TRP | C | 405 | 15.240 | 10.572 | −0.670 | 1.00 | 64.62 | C | |
| ATOM | 11166 | CB | TRP | C | 405 | 16.323 | 9.513 | −0.823 | 1.00 | 64.67 | C | |
| ATOM | 11169 | CG | TRP | C | 405 | 16.049 | 8.320 | −0.095 | 1.00 | 64.45 | C | |
| ATOM | 11170 | CD1 | TRP | C | 405 | 16.520 | 8.012 | 1.120 | 1.00 | 65.58 | C | |
| ATOM | 11172 | NE1 | TRP | C | 405 | 16.051 | 6.786 | 1.510 | 1.00 | 65.01 | N | |
| ATOM | 11174 | CE2 | TRP | C | 405 | 15.250 | 6.290 | 0.527 | 1.00 | 63.89 | C | |
| ATOM | 11175 | CD2 | TRP | C | 405 | 15.226 | 7.242 | −0.500 | 1.00 | 64.50 | C | |
| ATOM | 11176 | CE3 | TRP | C | 405 | 14.450 | 6.974 | −1.639 | 1.00 | 67.12 | C | |
| ATOM | 11178 | CZ3 | TRP | C | 405 | 13.739 | 5.750 | −1.714 | 1.00 | 65.92 | C | |
| ATOM | 11180 | CH2 | TRP | C | 405 | 13.802 | 4.824 | −0.670 | 1.00 | 65.18 | C | |
| ATOM | 11182 | CZ2 | TRP | C | 405 | 14.545 | 5.077 | 0.458 | 1.00 | 65.58 | C | |
| ATOM | 11184 | C | TRP | C | 405 | 15.861 | 11.904 | −1.082 | 1.00 | 63.51 | C | |
| ATOM | 11185 | O | TRP | C | 405 | 16.639 | 12.463 | −0.340 | 1.00 | 63.56 | O | |
| ATOM | 11187 | N | THR | C | 406 | 15.537 | 12.394 | −2.266 | 1.00 | 62.80 | N | |
| ATOM | 11188 | CA | THR | C | 406 | 16.234 | 13.526 | −2.872 | 1.00 | 62.12 | C | |
| ATOM | 11190 | CB | THR | C | 406 | 16.311 | 13.308 | −4.359 | 1.00 | 61.77 | C | |
| ATOM | 11192 | OG1 | THR | C | 406 | 16.999 | 12.068 | −4.619 | 1.00 | 62.35 | O | |
| ATOM | 11194 | CG2 | THR | C | 406 | 17.162 | 14.374 | −5.014 | 1.00 | 62.34 | C | |
| ATOM | 11198 | C | THR | C | 406 | 15.523 | 14.838 | −2.590 | 1.00 | 61.42 | C | |
| ATOM | 11199 | O | THR | C | 406 | 14.342 | 14.942 | −2.881 | 1.00 | 61.79 | O | |
| ATOM | 11201 | N | ALA | C | 407 | 16.284 | 15.823 | −2.101 | 1.00 | 60.77 | N | |
| ATOM | 11202 | CA | ALA | C | 407 | 15.812 | 17.123 | −1.636 | 1.00 | 60.47 | C | |
| ATOM | 11204 | CB | ALA | C | 407 | 16.955 | 17.906 | −1.038 | 1.00 | 60.58 | C | |
| ATOM | 11208 | C | ALA | C | 407 | 15.239 | 17.915 | −2.770 | 1.00 | 60.58 | C | |
| ATOM | 11209 | O | ALA | C | 407 | 15.708 | 17.750 | −3.867 | 1.00 | 60.96 | O | |
| ATOM | 11211 | N | PRO | C | 408 | 14.280 | 18.803 | −2.522 | 1.00 | 61.15 | N | |
| ATOM | 11212 | CA | PRO | C | 408 | 13.472 | 19.377 | −3.608 | 1.00 | 61.80 | C | |
| ATOM | 11214 | CB | PRO | C | 408 | 12.451 | 20.271 | −2.881 | 1.00 | 61.86 | C | |
| ATOM | 11217 | CG | PRO | C | 408 | 12.507 | 19.862 | −1.453 | 1.00 | 62.01 | C | |
| ATOM | 11220 | CD | PRO | C | 408 | 13.913 | 19.389 | −1.220 | 1.00 | 61.47 | C | |
| ATOM | 11223 | C | PRO | C | 408 | 14.297 | 20.189 | −4.601 | 1.00 | 62.35 | C | |
| ATOM | 11224 | O | PRO | C | 408 | 14.228 | 19.910 | −5.812 | 1.00 | 62.82 | O | |
| ATOM | 11225 | N | GLU | C | 409 | 15.114 | 21.127 | −4.109 | 1.00 | 62.31 | N | |
| ATOM | 11226 | CA | GLU | C | 409 | 16.021 | 21.857 | −5.012 | 1.00 | 62.90 | C | |
| ATOM | 11228 | CB | GLU | C | 409 | 16.902 | 22.887 | −4.293 | 1.00 | 63.27 | C | |
| ATOM | 11231 | CG | GLU | C | 409 | 18.113 | 22.355 | −3.525 | 1.00 | 64.22 | C | |
| ATOM | 11234 | CD | GLU | C | 409 | 17.762 | 21.616 | −2.264 | 1.00 | 65.78 | C | |
| ATOM | 11235 | OE1 | GLU | C | 409 | 16.585 | 21.657 | −1.816 | 1.00 | 67.23 | O | |
| ATOM | 11236 | OE2 | GLU | C | 409 | 18.701 | 21.013 | −1.718 | 1.00 | 66.21 | O | |
| ATOM | 11237 | C | GLU | C | 409 | 16.888 | 20.948 | −5.896 | 1.00 | 62.99 | C | |
| ATOM | 11238 | O | GLU | C | 409 | 17.278 | 21.347 | −6.989 | 1.00 | 62.76 | O | |
| ATOM | 11240 | N | SER | C | 410 | 17.192 | 19.743 | −5.415 | 1.00 | 63.15 | N | |
| ATOM | 11241 | CA | SER | C | 410 | 17.865 | 18.753 | −6.240 | 1.00 | 63.25 | C | |
| ATOM | 11243 | CB | SER | C | 410 | 18.477 | 17.663 | −5.410 | 1.00 | 63.19 | C | |
| ATOM | 11246 | OG | SER | C | 410 | 19.269 | 18.227 | −4.376 | 1.00 | 63.97 | O | |
| ATOM | 11248 | C | SER | C | 410 | 16.941 | 18.209 | −7.300 | 1.00 | 63.52 | C | |
| ATOM | 11249 | O | SER | C | 410 | 17.303 | 18.201 | −8.465 | 1.00 | 63.50 | O | |
| ATOM | 11251 | N | LEU | C | 411 | 15.731 | 17.821 | −6.926 | 1.00 | 63.76 | N | |
| ATOM | 11252 | CA | LEU | C | 411 | 14.762 | 17.304 | −7.917 | 1.00 | 64.03 | C | |
| ATOM | 11254 | CB | LEU | C | 411 | 13.449 | 16.909 | −7.253 | 1.00 | 64.14 | C | |
| ATOM | 11257 | CG | LEU | C | 411 | 13.414 | 15.864 | −6.162 | 1.00 | 64.13 | C | |
| ATOM | 11259 | CD1 | LEU | C | 411 | 11.990 | 15.856 | −5.602 | 1.00 | 64.88 | C | |
| ATOM | 11263 | CD2 | LEU | C | 411 | 13.827 | 14.511 | −6.689 | 1.00 | 63.93 | C | |
| ATOM | 11267 | C | LEU | C | 411 | 14.416 | 18.309 | −9.027 | 1.00 | 64.15 | C | |
| ATOM | 11268 | O | LEU | C | 411 | 14.305 | 17.931 | −10.190 | 1.00 | 63.79 | O | |
| ATOM | 11270 | N | ALA | C | 412 | 14.217 | 19.571 | −8.647 | 1.00 | 64.50 | N | |
| ATOM | 11271 | CA | ALA | C | 412 | 13.785 | 20.597 | −9.591 | 1.00 | 64.93 | C | |
| ATOM | 11273 | CB | ALA | C | 412 | 12.992 | 21.694 | −8.887 | 1.00 | 65.10 | C | |
| ATOM | 11277 | C | ALA | C | 412 | 15.013 | 21.156 | −10.279 | 1.00 | 65.29 | C | |
| ATOM | 11278 | O | ALA | C | 412 | 15.191 | 20.925 | −11.470 | 1.00 | 65.59 | O | |
| ATOM | 11280 | N | TYR | C | 413 | 15.889 | 21.802 | −9.493 | 1.00 | 65.68 | N | |
| ATOM | 11281 | CA | TYR | C | 413 | 17.092 | 22.540 | −9.975 | 1.00 | 65.29 | C | |
| ATOM | 11283 | CB | TYR | C | 413 | 17.447 | 23.672 | −8.945 | 1.00 | 66.55 | C | |
| ATOM | 11286 | CG | TYR | C | 413 | 18.031 | 24.943 | −9.522 | 1.00 | 67.76 | C | |
| ATOM | 11287 | CD1 | TYR | C | 413 | 19.430 | 25.064 | −9.731 | 1.00 | 69.64 | C | |
| ATOM | 11289 | CE1 | TYR | C | 413 | 20.002 | 26.240 | −10.304 | 1.00 | 69.05 | C | |
| ATOM | 11291 | CZ | TYR | C | 413 | 19.167 | 27.314 | −10.662 | 1.00 | 68.46 | C | |
| ATOM | 11292 | OH | TYR | C | 413 | 19.744 | 28.441 | −11.203 | 1.00 | 67.66 | O | |
| ATOM | 11294 | CE2 | TYR | C | 413 | 17.769 | 27.226 | −10.462 | 1.00 | 68.13 | C | |
| ATOM | 11296 | CD2 | TYR | C | 413 | 17.207 | 26.039 | −9.873 | 1.00 | 68.59 | C | |
| ATOM | 11298 | C | TYR | C | 413 | 18.332 | 21.635 | −10.303 | 1.00 | 64.82 | C | |
| ATOM | 11299 | O | TYR | C | 413 | 19.337 | 22.147 | −10.793 | 1.00 | 63.99 | O | |
| ATOM | 11301 | N | ASN | C | 414 | 18.247 | 20.309 | −10.043 | 1.00 | 64.70 | N | |
| ATOM | 11302 | CA | ASN | C | 414 | 19.322 | 19.296 | −10.289 | 1.00 | 64.39 | C | |
| ATOM | 11304 | CB | ASN | C | 414 | 19.393 | 18.954 | −11.797 | 1.00 | 64.22 | C | |
| ATOM | 11307 | CG | ASN | C | 414 | 19.396 | 17.421 | −12.092 | 1.00 | 65.28 | C | |
| ATOM | 11308 | OD1 | ASN | C | 414 | 19.724 | 16.597 | −11.254 | 1.00 | 69.93 | O | |
| ATOM | 11309 | ND2 | ASN | C | 414 | 19.037 | 17.061 | −13.300 | 1.00 | 66.74 | N | |
| ATOM | 11312 | C | ASN | C | 414 | 20.702 | 19.644 | −9.632 | 1.00 | 64.39 | C | |
| ATOM | 11313 | O | ASN | C | 414 | 21.788 | 19.360 | −10.171 | 1.00 | 64.68 | O | |
| ATOM | 11315 | N | LYS | C | 415 | 20.611 | 20.251 | −8.445 | 1.00 | 63.86 | N | |
| ATOM | 11316 | CA | LYS | C | 415 | 21.721 | 20.904 | −7.742 | 1.00 | 63.81 | C | |
| ATOM | 11318 | CB | LYS | C | 415 | 21.437 | 22.421 | −7.596 | 1.00 | 63.79 | C | |
| ATOM | 11321 | CG | LYS | C | 415 | 22.135 | 23.135 | −6.425 | 1.00 | 63.57 | C | |
| ATOM | 11324 | CD | LYS | C | 415 | 22.243 | 24.640 | −6.601 | 1.00 | 64.50 | C | |
| ATOM | 11327 | CE | LYS | C | 415 | 20.908 | 25.402 | −6.506 | 1.00 | 65.40 | C | |
| ATOM | 11330 | NZ | LYS | C | 415 | 21.060 | 26.771 | −7.204 | 1.00 | 63.55 | N | |
| ATOM | 11334 | C | LYS | C | 415 | 21.845 | 20.248 | −6.388 | 1.00 | 63.23 | C | |
| ATOM | 11335 | O | LYS | C | 415 | 20.854 | 20.063 | −5.710 | 1.00 | 63.74 | O | |
| ATOM | 11337 | N | PHE | C | 416 | 23.060 | 19.934 | −5.980 | 1.00 | 62.99 | N | |
| ATOM | 11338 | CA | PHE | C | 416 | 23.292 | 19.177 | −4.764 | 1.00 | 62.98 | C | |
| ATOM | 11340 | CB | PHE | C | 416 | 23.854 | 17.811 | −5.116 | 1.00 | 63.16 | C | |
| ATOM | 11343 | CG | PHE | C | 416 | 22.850 | 16.910 | −5.759 | 1.00 | 61.73 | C | |
| ATOM | 11344 | CD1 | PHE | C | 416 | 22.690 | 16.893 | −7.125 | 1.00 | 62.22 | C | |
| ATOM | 11346 | CE1 | PHE | C | 416 | 21.758 | 16.076 | −7.730 | 1.00 | 61.97 | C | |
| ATOM | 11348 | CZ | PHE | C | 416 | 20.981 | 15.232 | −6.962 | 1.00 | 63.09 | C | |
| ATOM | 11350 | CE2 | PHE | C | 416 | 21.135 | 15.225 | −5.590 | 1.00 | 64.18 | C | |
| ATOM | 11352 | CD2 | PHE | C | 416 | 22.065 | 16.084 | −4.995 | 1.00 | 63.42 | C | |
| ATOM | 11354 | C | PHE | C | 416 | 24.230 | 19.944 | −3.872 | 1.00 | 63.57 | C | |
| ATOM | 11355 | O | PHE | C | 416 | 25.089 | 20.686 | −4.353 | 1.00 | 63.90 | O | |
| ATOM | 11357 | N | SER | C | 417 | 24.012 | 19.811 | −2.568 | 1.00 | 64.17 | N | |
| ATOM | 11358 | CA | SER | C | 417 | 24.756 | 20.573 | −1.528 | 1.00 | 64.65 | C | |
| ATOM | 11360 | CB | SER | C | 417 | 24.024 | 21.868 | −1.089 | 1.00 | 64.47 | C | |
| ATOM | 11363 | OG | SER | C | 417 | 22.686 | 21.628 | −0.683 | 1.00 | 63.36 | O | |
| ATOM | 11365 | C | SER | C | 417 | 24.949 | 19.641 | −0.373 | 1.00 | 64.77 | C | |
| ATOM | 11366 | O | SER | C | 417 | 24.451 | 18.516 | −0.393 | 1.00 | 66.61 | O | |
| ATOM | 11368 | N | ILE | C | 418 | 25.695 | 20.056 | 0.620 | 1.00 | 64.58 | N | |
| ATOM | 11369 | CA | ILE | C | 418 | 25.841 | 19.221 | 1.803 | 1.00 | 65.24 | C | |
| ATOM | 11371 | CB | ILE | C | 418 | 27.126 | 19.634 | 2.597 | 1.00 | 65.52 | C | |
| ATOM | 11373 | CG | ILE | C | 418 | 27.825 | 18.414 | 3.190 | 1.00 | 65.99 | C | |
| ATOM | 11376 | CD1 | ILE | C | 418 | 26.993 | 17.587 | 4.069 | 1.00 | 66.69 | C | |
| ATOM | 11380 | CG2 | ILE | C | 418 | 26.866 | 20.795 | 3.589 | 1.00 | 66.57 | C | |
| ATOM | 11384 | C | ILE | C | 418 | 24.496 | 19.256 | 2.574 | 1.00 | 65.30 | C | |
| ATOM | 11385 | O | ILE | C | 418 | 24.092 | 18.278 | 3.197 | 1.00 | 65.56 | O | |
| ATOM | 11387 | N | LYS | C | 419 | 23.794 | 20.387 | 2.455 | 1.00 | 65.61 | N | |
| ATOM | 11388 | CA | LYS | C | 419 | 22.362 | 20.529 | 2.822 | 1.00 | 65.17 | C | |
| ATOM | 11390 | CB | LYS | C | 419 | 21.902 | 22.011 | 2.643 | 1.00 | 64.70 | C | |
| ATOM | 11393 | CG | LYS | C | 419 | 22.002 | 22.901 | 3.904 | 1.00 | 64.72 | C | |
| ATOM | 11396 | CD | LYS | C | 419 | 23.183 | 22.584 | 4.862 | 1.00 | 65.40 | C | |
| ATOM | 11399 | CE | LYS | C | 419 | 23.324 | 23.640 | 5.973 | 1.00 | 65.56 | C | |
| ATOM | 11402 | NZ | LYS | C | 419 | 24.144 | 23.236 | 7.162 | 1.00 | 65.98 | N | |
| ATOM | 11406 | C | LYS | C | 419 | 21.390 | 19.537 | 2.147 | 1.00 | 64.78 | C | |
| ATOM | 11407 | O | LYS | C | 419 | 20.423 | 19.128 | 2.771 | 1.00 | 64.66 | O | |
| ATOM | 11409 | N | SER | C | 420 | 21.643 | 19.155 | 0.902 | 1.00 | 64.79 | N | |
| ATOM | 11410 | CA | SER | C | 420 | 20.840 | 18.127 | 0.244 | 1.00 | 65.00 | C | |
| ATOM | 11412 | CB | SER | C | 420 | 21.082 | 18.090 | −1.256 | 1.00 | 65.01 | C | |
| ATOM | 11415 | OG | SER | C | 420 | 22.237 | 17.329 | −1.552 | 1.00 | 65.99 | O | |
| ATOM | 11417 | C | SER | C | 420 | 21.108 | 16.741 | 0.832 | 1.00 | 65.13 | C | |
| ATOM | 11418 | O | SER | C | 420 | 20.180 | 15.937 | 0.877 | 1.00 | 65.68 | O | |
| ATOM | 11420 | N | ASP | C | 421 | 22.359 | 16.452 | 1.232 | 1.00 | 64.87 | N | |
| ATOM | 11421 | CA | ASP | C | 421 | 22.668 | 15.238 | 2.063 | 1.00 | 64.62 | C | |
| ATOM | 11423 | CB | ASP | C | 421 | 24.188 | 15.056 | 2.284 | 1.00 | 64.75 | C | |
| ATOM | 11426 | CG | ASP | C | 421 | 24.875 | 14.179 | 1.210 | 1.00 | 66.91 | C | |
| ATOM | 11427 | OD1 | ASP | C | 421 | 24.308 | 13.811 | 0.143 | 1.00 | 65.03 | O | |
| ATOM | 11428 | OD2 | ASP | C | 421 | 26.070 | 13.823 | 1.363 | 1.00 | 71.00 | O | |
| ATOM | 11429 | C | ASP | C | 421 | 21.967 | 15.247 | 3.435 | 1.00 | 64.01 | C | |
| ATOM | 11430 | O | ASP | C | 421 | 21.677 | 14.179 | 3.999 | 1.00 | 62.96 | O | |
| ATOM | 11432 | N | VAL | C | 422 | 21.731 | 16.451 | 3.972 | 1.00 | 63.60 | N | |
| ATOM | 11433 | CA | VAL | C | 422 | 21.019 | 16.628 | 5.252 | 1.00 | 63.45 | C | |
| ATOM | 11435 | CB | VAL | C | 422 | 21.172 | 18.101 | 5.807 | 1.00 | 63.50 | C | |
| ATOM | 11437 | CG1 | VAL | C | 422 | 20.125 | 18.478 | 6.875 | 1.00 | 63.36 | C | |
| ATOM | 11441 | CG2 | VAL | C | 422 | 22.569 | 18.282 | 6.354 | 1.00 | 63.53 | C | |
| ATOM | 11445 | C | VAL | C | 422 | 19.570 | 16.166 | 5.108 | 1.00 | 62.50 | C | |
| ATOM | 11446 | O | VAL | C | 422 | 19.112 | 15.324 | 5.869 | 1.00 | 62.85 | O | |
| ATOM | 11448 | N | TRP | C | 423 | 18.877 | 16.670 | 4.106 | 1.00 | 61.37 | N | |
| ATOM | 11449 | CA | TRP | C | 423 | 17.556 | 16.158 | 3.768 | 1.00 | 60.68 | C | |
| ATOM | 11451 | CB | TRP | C | 423 | 17.077 | 16.748 | 2.448 | 1.00 | 60.79 | C | |
| ATOM | 11454 | CG | TRP | C | 423 | 15.678 | 16.387 | 2.096 | 1.00 | 60.20 | C | |
| ATOM | 11455 | CD1 | TRP | C | 423 | 15.198 | 15.157 | 1.722 | 1.00 | 60.05 | C | |
| ATOM | 11457 | NE1 | TRP | C | 423 | 13.849 | 15.226 | 1.496 | 1.00 | 60.53 | N | |
| ATOM | 11459 | CE2 | TRP | C | 423 | 13.445 | 16.518 | 1.699 | 1.00 | 60.68 | C | |
| ATOM | 11460 | CD2 | TRP | C | 423 | 14.585 | 17.265 | 2.075 | 1.00 | 59.41 | C | |
| ATOM | 11461 | CE3 | TRP | C | 423 | 14.435 | 18.619 | 2.353 | 1.00 | 60.05 | C | |
| ATOM | 11463 | CZ3 | TRP | C | 423 | 13.167 | 19.192 | 2.247 | 1.00 | 60.92 | C | |
| ATOM | 11465 | CH2 | TRP | C | 423 | 12.046 | 18.422 | 1.866 | 1.00 | 60.42 | C | |
| ATOM | 11467 | CZ2 | TRP | C | 423 | 12.164 | 17.092 | 1.584 | 1.00 | 60.72 | C | |
| ATOM | 11469 | C | TRP | C | 423 | 17.564 | 14.630 | 3.665 | 1.00 | 60.18 | C | |
| ATOM | 11470 | O | TRP | C | 423 | 16.725 | 13.966 | 4.300 | 1.00 | 60.57 | O | |
| ATOM | 11472 | N | ALA | C | 424 | 18.488 | 14.088 | 2.872 | 1.00 | 59.04 | N | |
| ATOM | 11473 | CA | ALA | C | 424 | 18.608 | 12.633 | 2.728 | 1.00 | 59.02 | C | |
| ATOM | 11475 | CB | ALA | C | 424 | 19.723 | 12.287 | 1.796 | 1.00 | 58.35 | C | |
| ATOM | 11479 | C | ALA | C | 424 | 18.804 | 11.917 | 4.089 | 1.00 | 59.41 | C | |
| ATOM | 11480 | O | ALA | C | 424 | 18.193 | 10.865 | 4.343 | 1.00 | 59.00 | O | |
| ATOM | 11482 | N | PHE | C | 425 | 19.641 | 12.510 | 4.960 | 1.00 | 59.99 | N | |
| ATOM | 11483 | CA | PHE | C | 425 | 19.869 | 11.987 | 6.331 | 1.00 | 60.03 | C | |
| ATOM | 11485 | CB | PHE | C | 425 | 20.888 | 12.820 | 7.128 | 1.00 | 59.91 | C | |
| ATOM | 11488 | CG | PHE | C | 425 | 21.224 | 12.231 | 8.475 | 1.00 | 59.16 | C | |
| ATOM | 11489 | CD1 | PHE | C | 425 | 22.074 | 11.138 | 8.577 | 1.00 | 57.62 | C | |
| ATOM | 11491 | CE1 | PHE | C | 425 | 22.385 | 10.583 | 9.830 | 1.00 | 57.67 | C | |
| ATOM | 11493 | CZ | PHE | C | 425 | 21.812 | 11.107 | 10.977 | 1.00 | 58.54 | C | |
| ATOM | 11495 | CE2 | PHE | C | 425 | 20.953 | 12.192 | 10.892 | 1.00 | 58.25 | C | |
| ATOM | 11497 | CD2 | PHE | C | 425 | 20.683 | 12.768 | 9.642 | 1.00 | 59.67 | C | |
| ATOM | 11499 | C | PHE | C | 425 | 18.569 | 11.931 | 7.110 | 1.00 | 60.64 | C | |
| ATOM | 11500 | O | PHE | C | 425 | 18.291 | 10.899 | 7.761 | 1.00 | 61.54 | O | |
| ATOM | 11502 | N | GLY | C | 426 | 17.782 | 13.017 | 7.019 | 1.00 | 60.36 | N | |
| ATOM | 11503 | CA | GLY | C | 426 | 16.415 | 13.066 | 7.557 | 1.00 | 60.23 | C | |
| ATOM | 11506 | C | GLY | C | 426 | 15.568 | 11.865 | 7.162 | 1.00 | 60.14 | C | |
| ATOM | 11507 | O | GLY | C | 426 | 14.963 | 11.247 | 8.014 | 1.00 | 59.96 | O | |
| ATOM | 11509 | N | VAL | C | 427 | 15.569 | 11.520 | 5.872 | 1.00 | 60.35 | N | |
| ATOM | 11510 | CA | VAL | C | 427 | 14.756 | 10.425 | 5.349 | 1.00 | 60.65 | C | |
| ATOM | 11512 | CB | VAL | C | 427 | 14.727 | 10.377 | 3.798 | 1.00 | 60.79 | C | |
| ATOM | 11514 | CG1 | VAL | C | 427 | 14.020 | 9.087 | 3.261 | 1.00 | 60.27 | C | |
| ATOM | 11518 | CG2 | VAL | C | 427 | 14.028 | 11.611 | 3.252 | 1.00 | 59.88 | C | |
| ATOM | 11522 | C | VAL | C | 427 | 15.304 | 9.143 | 5.865 | 1.00 | 61.60 | C | |
| ATOM | 11523 | O | VAL | C | 427 | 14.541 | 8.252 | 6.252 | 1.00 | 61.44 | O | |
| ATOM | 11525 | N | LEU | C | 428 | 16.633 | 9.048 | 5.869 | 1.00 | 63.04 | N | |
| ATOM | 11526 | CA | LEU | C | 428 | 17.318 | 7.884 | 6.450 | 1.00 | 63.78 | C | |
| ATOM | 11528 | CB | LEU | C | 428 | 18.837 | 8.032 | 6.316 | 1.00 | 63.91 | C | |
| ATOM | 11531 | CG | LEU | C | 428 | 19.753 | 7.050 | 7.039 | 1.00 | 63.83 | C | |
| ATOM | 11533 | CD1 | LEU | C | 428 | 19.481 | 5.669 | 6.545 | 1.00 | 65.35 | C | |
| ATOM | 11537 | CD2 | LEU | C | 428 | 21.219 | 7.426 | 6.782 | 1.00 | 64.37 | C | |
| ATOM | 11541 | C | LEU | C | 428 | 16.912 | 7.659 | 7.905 | 1.00 | 64.10 | C | |
| ATOM | 11542 | O | LEU | C | 428 | 16.733 | 6.515 | 8.301 | 1.00 | 64.36 | O | |
| ATOM | 11544 | N | LEU | C | 429 | 16.741 | 8.732 | 8.692 | 1.00 | 64.22 | N | |
| ATOM | 11545 | CA | LEU | C | 429 | 16.229 | 8.553 | 10.056 | 1.00 | 64.70 | C | |
| ATOM | 11547 | CB | LEU | C | 429 | 16.039 | 9.852 | 10.831 | 1.00 | 64.27 | C | |
| ATOM | 11550 | CG | LEU | C | 429 | 17.265 | 10.653 | 11.206 | 1.00 | 64.56 | C | |
| ATOM | 11552 | CD1 | LEU | C | 429 | 16.773 | 11.829 | 12.074 | 1.00 | 66.15 | C | |
| ATOM | 11556 | CD2 | LEU | C | 429 | 18.326 | 9.836 | 11.951 | 1.00 | 62.69 | C | |
| ATOM | 11560 | C | LEU | C | 429 | 14.900 | 7.836 | 10.030 | 1.00 | 65.09 | C | |
| ATOM | 11561 | O | LEU | C | 429 | 14.733 | 6.855 | 10.750 | 1.00 | 65.48 | O | |
| ATOM | 11563 | N | TRP | C | 430 | 13.996 | 8.329 | 9.176 | 1.00 | 65.33 | N | |
| ATOM | 11564 | CA | TRP | C | 430 | 12.631 | 7.820 | 9.059 | 1.00 | 65.18 | C | |
| ATOM | 11566 | CB | TRP | C | 430 | 11.816 | 8.641 | 8.039 | 1.00 | 65.11 | C | |
| ATOM | 11569 | CG | TRP | C | 430 | 10.343 | 8.417 | 8.125 | 1.00 | 65.31 | C | |
| ATOM | 11570 | CD1 | TRP | C | 430 | 9.414 | 9.164 | 8.812 | 1.00 | 64.90 | C | |
| ATOM | 11572 | NE1 | TRP | C | 430 | 8.157 | 8.628 | 8.652 | 1.00 | 64.23 | N | |
| ATOM | 11574 | CE2 | TRP | C | 430 | 8.253 | 7.516 | 7.856 | 1.00 | 64.09 | C | |
| ATOM | 11575 | CD2 | TRP | C | 430 | 9.618 | 7.359 | 7.501 | 1.00 | 65.33 | C | |
| ATOM | 11576 | CE3 | TRP | C | 430 | 9.985 | 6.281 | 6.667 | 1.00 | 64.19 | C | |
| ATOM | 11578 | CZ3 | TRP | C | 430 | 9.006 | 5.425 | 6.216 | 1.00 | 64.96 | C | |
| ATOM | 11580 | CH2 | TRP | C | 430 | 7.652 | 5.616 | 6.584 | 1.00 | 65.28 | C | |
| ATOM | 11582 | CZ2 | TRP | C | 430 | 7.264 | 6.658 | 7.399 | 1.00 | 64.42 | C | |
| ATOM | 11584 | C | TRP | C | 430 | 12.670 | 6.338 | 8.734 | 1.00 | 65.08 | C | |
| ATOM | 11585 | O | TRP | C | 430 | 12.027 | 5.565 | 9.424 | 1.00 | 65.21 | O | |
| ATOM | 11587 | N | GLU | C | 431 | 13.493 | 5.931 | 7.771 | 1.00 | 65.64 | N | |
| ATOM | 11588 | CA | GLU | C | 431 | 13.718 | 4.484 | 7.514 | 1.00 | 66.77 | C | |
| ATOM | 11590 | CB | GLU | C | 431 | 14.783 | 4.217 | 6.449 | 1.00 | 66.67 | C | |
| ATOM | 11593 | CG | GLU | C | 431 | 14.378 | 4.435 | 4.994 | 1.00 | 67.87 | C | |
| ATOM | 11596 | CD | GLU | C | 431 | 15.589 | 4.299 | 4.062 | 1.00 | 69.49 | C | |
| ATOM | 11597 | OE1 | GLU | C | 431 | 16.544 | 5.131 | 4.187 | 1.00 | 72.37 | O | |
| ATOM | 11598 | OE2 | GLU | C | 431 | 15.630 | 3.342 | 3.230 | 1.00 | 74.52 | O | |
| ATOM | 11599 | C | GLU | C | 431 | 14.135 | 3.764 | 8.790 | 1.00 | 66.48 | C | |
| ATOM | 11600 | O | GLU | C | 431 | 13.500 | 2.773 | 9.144 | 1.00 | 66.86 | O | |
| ATOM | 11602 | N | ILE | C | 432 | 15.153 | 4.285 | 9.493 | 1.00 | 66.47 | N | |
| ATOM | 11603 | CA | ILE | C | 432 | 15.586 | 3.679 | 10.766 | 1.00 | 66.46 | C | |
| ATOM | 11605 | CB | ILE | C | 432 | 16.751 | 4.416 | 11.469 | 1.00 | 66.66 | C | |
| ATOM | 11607 | CG1 | ILE | C | 432 | 18.040 | 4.318 | 10.652 | 1.00 | 67.24 | C | |
| ATOM | 11610 | CD1 | ILE | C | 432 | 19.270 | 5.023 | 11.265 | 1.00 | 66.69 | C | |
| ATOM | 11614 | CG2 | ILE | C | 432 | 17.019 | 3.767 | 12.860 | 1.00 | 66.74 | C | |
| ATOM | 11618 | C | ILE | C | 432 | 14.438 | 3.580 | 11.741 | 1.00 | 66.30 | C | |
| ATOM | 11619 | O | ILE | C | 432 | 14.201 | 2.504 | 12.247 | 1.00 | 66.62 | O | |
| ATOM | 11621 | N | ALA | C | 433 | 13.776 | 4.714 | 12.008 | 1.00 | 66.57 | N | |
| ATOM | 11622 | CA | ALA | C | 433 | 12.605 | 4.827 | 12.911 | 1.00 | 66.43 | C | |
| ATOM | 11624 | CB | ALA | C | 433 | 11.978 | 6.198 | 12.808 | 1.00 | 66.42 | C | |
| ATOM | 11628 | C | ALA | C | 433 | 11.551 | 3.768 | 12.660 | 1.00 | 66.85 | C | |
| ATOM | 11629 | O | ALA | C | 433 | 11.168 | 3.039 | 13.578 | 1.00 | 67.30 | O | |
| ATOM | 11631 | N | THR | C | 434 | 11.167 | 3.609 | 11.397 | 1.00 | 67.29 | N | |
| ATOM | 11632 | CA | THR | C | 434 | 10.130 | 2.621 | 11.000 | 1.00 | 67.68 | C | |
| ATOM | 11634 | CB | THR | C | 434 | 9.569 | 3.023 | 9.653 | 1.00 | 67.35 | C | |
| ATOM | 11636 | OG1 | THR | C | 434 | 10.649 | 3.146 | 8.720 | 1.00 | 65.05 | O | |
| ATOM | 11638 | CG2 | THR | C | 434 | 8.922 | 4.411 | 9.720 | 1.00 | 67.46 | C | |
| ATOM | 11642 | C | THR | C | 434 | 10.579 | 1.156 | 10.872 | 1.00 | 68.37 | C | |
| ATOM | 11643 | O | THR | C | 434 | 9.754 | 0.314 | 10.524 | 1.00 | 68.47 | O | |
| ATOM | 11645 | N | TYR | C | 435 | 11.859 | 0.860 | 11.140 | 1.00 | 69.30 | N | |
| ATOM | 11646 | CA | TYR | C | 435 | 12.511 | −0.399 | 10.767 | 1.00 | 69.71 | C | |
| ATOM | 11648 | CB | TYR | C | 435 | 12.085 | −1.533 | 11.703 | 1.00 | 70.66 | C | |
| ATOM | 11651 | CG | TYR | C | 435 | 12.405 | −1.389 | 13.176 | 1.00 | 71.44 | C | |
| ATOM | 11652 | CD1 | TYR | C | 435 | 11.530 | −0.717 | 14.046 | 1.00 | 71.73 | C | |
| ATOM | 11654 | CE1 | TYR | C | 435 | 11.801 | −0.635 | 15.422 | 1.00 | 71.50 | C | |
| ATOM | 11656 | CZ | TYR | C | 435 | 12.955 | −1.240 | 15.942 | 1.00 | 71.43 | C | |
| ATOM | 11657 | OH | TYR | C | 435 | 13.233 | −1.159 | 17.285 | 1.00 | 70.91 | O | |
| ATOM | 11659 | CE2 | TYR | C | 435 | 13.837 | −1.908 | 15.102 | 1.00 | 71.38 | C | |
| ATOM | 11661 | CD2 | TYR | C | 435 | 13.551 | −1.994 | 13.725 | 1.00 | 71.86 | C | |
| ATOM | 11663 | C | TYR | C | 435 | 12.242 | −0.812 | 9.310 | 1.00 | 70.16 | C | |
| ATOM | 11664 | O | TYR | C | 435 | 11.748 | −1.917 | 9.038 | 1.00 | 70.08 | O | |
| ATOM | 11666 | N | GLY | C | 436 | 12.522 | 0.107 | 8.389 | 1.00 | 70.74 | N | |
| ATOM | 11667 | CA | GLY | C | 436 | 12.554 | −0.182 | 6.942 | 1.00 | 71.05 | C | |
| ATOM | 11670 | C | GLY | C | 436 | 11.288 | −0.010 | 6.112 | 1.00 | 71.37 | C | |
| ATOM | 11671 | O | GLY | C | 436 | 11.169 | −0.636 | 5.068 | 1.00 | 71.52 | O | |
| ATOM | 11673 | N | MET | C | 437 | 10.354 | 0.846 | 6.532 | 1.00 | 71.88 | N | |
| ATOM | 11674 | CA | MET | C | 437 | 9.205 | 1.230 | 5.669 | 1.00 | 72.07 | C | |
| ATOM | 11676 | CB | MET | C | 437 | 8.093 | 1.942 | 6.466 | 1.00 | 72.35 | C | |
| ATOM | 11679 | CG | MET | C | 437 | 7.304 | 1.032 | 7.388 | 1.00 | 73.14 | C | |
| ATOM | 11682 | SD | MET | C | 437 | 5.718 | 0.449 | 6.710 | 1.00 | 77.41 | S | |
| ATOM | 11683 | CE | MET | C | 437 | 6.067 | −0.178 | 4.944 | 1.00 | 76.82 | C | |
| ATOM | 11687 | C | MET | C | 437 | 9.624 | 2.111 | 4.462 | 1.00 | 72.11 | C | |
| ATOM | 11688 | O | MET | C | 437 | 10.638 | 2.812 | 4.486 | 1.00 | 70.63 | O | |
| ATOM | 11690 | N | SER | C | 438 | 8.811 | 2.028 | 3.410 | 1.00 | 72.63 | N | |
| ATOM | 11691 | CA | SER | C | 438 | 9.010 | 2.809 | 2.205 | 1.00 | 72.91 | C | |
| ATOM | 11693 | CB | SER | C | 438 | 8.191 | 2.267 | 1.024 | 1.00 | 73.08 | C | |
| ATOM | 11696 | OG | SER | C | 438 | 9.050 | 1.809 | −0.003 | 1.00 | 74.83 | O | |
| ATOM | 11698 | C | SER | C | 438 | 8.593 | 4.236 | 2.534 | 1.00 | 72.89 | C | |
| ATOM | 11699 | O | SER | C | 438 | 7.476 | 4.461 | 3.031 | 1.00 | 73.11 | O | |
| ATOM | 11701 | N | PRO | C | 439 | 9.470 | 5.203 | 2.296 | 1.00 | 72.80 | N | |
| ATOM | 11702 | CA | PRO | C | 439 | 9.097 | 6.606 | 2.504 | 1.00 | 72.72 | C | |
| ATOM | 11704 | CB | PRO | C | 439 | 10.348 | 7.387 | 2.125 | 1.00 | 72.54 | C | |
| ATOM | 11707 | CG | PRO | C | 439 | 11.441 | 6.388 | 1.993 | 1.00 | 72.87 | C | |
| ATOM | 11710 | CD | PRO | C | 439 | 10.877 | 5.047 | 1.887 | 1.00 | 72.60 | C | |
| ATOM | 11713 | C | PRO | C | 439 | 7.895 | 7.063 | 1.685 | 1.00 | 72.94 | C | |
| ATOM | 11714 | O | PRO | C | 439 | 7.567 | 6.447 | 0.667 | 1.00 | 72.60 | O | |
| ATOM | 11715 | N | TYR | C | 440 | 7.244 | 8.120 | 2.187 | 1.00 | 73.33 | N | |
| ATOM | 11716 | CA | TYR | C | 440 | 5.986 | 8.680 | 1.650 | 1.00 | 73.29 | C | |
| ATOM | 11718 | CB | TYR | C | 440 | 6.280 | 9.555 | 0.460 | 1.00 | 72.16 | C | |
| ATOM | 11721 | CG | TYR | C | 440 | 7.163 | 10.684 | 0.865 | 1.00 | 70.93 | C | |
| ATOM | 11722 | CD1 | TYR | C | 440 | 6.627 | 11.844 | 1.412 | 1.00 | 69.86 | C | |
| ATOM | 11724 | CE1 | TYR | C | 440 | 7.445 | 12.891 | 1.799 | 1.00 | 69.75 | C | |
| ATOM | 11726 | CZ | TYR | C | 440 | 8.807 | 12.755 | 1.657 | 1.00 | 69.27 | C | |
| ATOM | 11727 | OH | TYR | C | 440 | 9.648 | 13.757 | 2.022 | 1.00 | 70.36 | O | |
| ATOM | 11729 | CE2 | TYR | C | 440 | 9.348 | 11.613 | 1.125 | 1.00 | 68.81 | C | |
| ATOM | 11731 | CD2 | TYR | C | 440 | 8.541 | 10.588 | 0.741 | 1.00 | 69.21 | C | |
| ATOM | 11733 | C | TYR | C | 440 | 4.973 | 7.566 | 1.341 | 1.00 | 74.44 | C | |
| ATOM | 11734 | O | TYR | C | 440 | 4.634 | 7.306 | 0.177 | 1.00 | 74.58 | O | |
| ATOM | 11736 | N | PRO | C | 441 | 4.499 | 6.922 | 2.414 | 1.00 | 75.25 | N | |
| ATOM | 11737 | CA | PRO | C | 441 | 4.168 | 5.498 | 2.356 | 1.00 | 75.45 | C | |
| ATOM | 11739 | CB | PRO | C | 441 | 3.908 | 5.136 | 3.829 | 1.00 | 75.52 | C | |
| ATOM | 11742 | CG | PRO | C | 441 | 3.434 | 6.432 | 4.472 | 1.00 | 75.73 | C | |
| ATOM | 11745 | CD | PRO | C | 441 | 4.162 | 7.523 | 3.726 | 1.00 | 75.29 | C | |
| ATOM | 11748 | C | PRO | C | 441 | 2.943 | 5.272 | 1.487 | 1.00 | 75.26 | C | |
| ATOM | 11749 | O | PRO | C | 441 | 1.896 | 5.849 | 1.785 | 1.00 | 75.41 | O | |
| ATOM | 11750 | N | GLY | C | 442 | 3.107 | 4.516 | 0.390 | 1.00 | 74.66 | N | |
| ATOM | 11751 | CA | GLY | C | 442 | 2.006 | 4.244 | −0.553 | 1.00 | 73.91 | C | |
| ATOM | 11754 | C | GLY | C | 442 | 1.546 | 5.350 | −1.512 | 1.00 | 73.50 | C | |
| ATOM | 11755 | O | GLY | C | 442 | 0.620 | 5.106 | −2.264 | 1.00 | 73.42 | O | |
| ATOM | 11757 | N | ILE | C | 443 | 2.173 | 6.536 | −1.484 | 1.00 | 72.83 | N | |
| ATOM | 11758 | CA | ILE | C | 443 | 1.964 | 7.623 | −2.465 | 1.00 | 72.68 | C | |
| ATOM | 11760 | CB | ILE | C | 443 | 2.508 | 8.994 | −1.877 | 1.00 | 72.68 | C | |
| ATOM | 11762 | CG1 | ILE | C | 443 | 1.696 | 9.415 | −0.643 | 1.00 | 73.33 | C | |
| ATOM | 11765 | CD1 | ILE | C | 443 | 2.469 | 10.303 | 0.333 | 1.00 | 73.03 | C | |
| ATOM | 11769 | CG2 | ILE | C | 443 | 2.533 | 10.148 | −2.908 | 1.00 | 71.95 | C | |
| ATOM | 11773 | C | ILE | C | 443 | 2.702 | 7.278 | −3.778 | 1.00 | 72.68 | C | |
| ATOM | 11774 | O | ILE | C | 443 | 3.798 | 6.689 | −3.736 | 1.00 | 73.04 | O | |
| ATOM | 11776 | N | ASP | C | 444 | 2.103 | 7.653 | −4.924 | 1.00 | 72.20 | N | |
| ATOM | 11777 | CA | ASP | C | 444 | 2.781 | 7.666 | −6.249 | 1.00 | 71.48 | C | |
| ATOM | 11779 | CB | ASP | C | 444 | 1.748 | 7.630 | −7.388 | 1.00 | 71.56 | C | |
| ATOM | 11782 | CG | ASP | C | 444 | 2.346 | 7.189 | −8.740 | 1.00 | 71.74 | C | |
| ATOM | 11783 | OD1 | ASP | C | 444 | 3.204 | 7.909 | −9.324 | 1.00 | 70.76 | O | |
| ATOM | 11784 | OD2 | ASP | C | 444 | 1.971 | 6.134 | −9.304 | 1.00 | 70.66 | O | |
| ATOM | 11785 | C | ASP | C | 444 | 3.702 | 8.901 | −6.427 | 1.00 | 71.12 | C | |
| ATOM | 11786 | O | ASP | C | 444 | 3.314 | 10.059 | −6.125 | 1.00 | 71.08 | O | |
| ATOM | 11788 | N | LEU | C | 445 | 4.869 | 8.639 | −7.030 | 1.00 | 70.44 | N | |
| ATOM | 11789 | CA | LEU | C | 445 | 6.037 | 9.556 | −7.048 | 1.00 | 69.70 | C | |
| ATOM | 11791 | CB | LEU | C | 445 | 7.239 | 8.884 | −7.743 | 1.00 | 69.72 | C | |
| ATOM | 11794 | CG | LEU | C | 445 | 7.709 | 7.414 | −7.577 | 1.00 | 69.95 | C | |
| ATOM | 11796 | CD1 | LEU | C | 445 | 8.511 | 6.945 | −8.799 | 1.00 | 70.38 | C | |
| ATOM | 11800 | CD2 | LEU | C | 445 | 8.549 | 7.202 | −6.353 | 1.00 | 70.42 | C | |
| ATOM | 11804 | C | LEU | C | 445 | 5.808 | 10.916 | −7.736 | 1.00 | 69.14 | C | |
| ATOM | 11805 | O | LEU | C | 445 | 6.550 | 11.872 | −7.499 | 1.00 | 68.61 | O | |
| ATOM | 11807 | N | SER | C | 446 | 4.818 | 10.973 | −8.628 | 1.00 | 68.94 | N | |
| ATOM | 11808 | CA | SER | C | 446 | 4.480 | 12.185 | −9.408 | 1.00 | 68.68 | C | |
| ATOM | 11810 | CB | SER | C | 446 | 3.322 | 11.878 | −10.377 | 1.00 | 68.33 | C | |
| ATOM | 11813 | OG | SER | C | 446 | 2.610 | 10.707 | −9.993 | 1.00 | 66.71 | O | |
| ATOM | 11815 | C | SER | C | 446 | 4.179 | 13.435 | −8.562 | 1.00 | 68.68 | C | |
| ATOM | 11816 | O | SER | C | 446 | 4.671 | 14.521 | −8.878 | 1.00 | 68.00 | O | |
| ATOM | 11818 | N | GLN | C | 447 | 3.412 | 13.250 | −7.477 | 1.00 | 69.14 | N | |
| ATOM | 11819 | CA | GLN | C | 447 | 2.890 | 14.370 | −6.643 | 1.00 | 69.10 | C | |
| ATOM | 11821 | CB | GLN | C | 447 | 1.428 | 14.121 | −6.220 | 1.00 | 69.68 | C | |
| ATOM | 11824 | CG | GLN | C | 447 | 0.381 | 14.835 | −7.112 | 1.00 | 69.97 | C | |
| ATOM | 11827 | CD | GLN | C | 447 | 0.572 | 13.881 | −7.777 | 1.00 | 71.82 | C | |
| ATOM | 11828 | OE1 | GLN | C | 447 | 1.789 | 14.051 | −7.689 | 1.00 | 74.10 | O | |
| ATOM | 11829 | NE2 | GLN | C | 447 | 0.023 | 12.880 | −8.468 | 1.00 | 72.26 | N | |
| ATOM | 11832 | C | GLN | C | 447 | 3.716 | 14.718 | −5.427 | 1.00 | 68.57 | C | |
| ATOM | 11833 | O | GLN | C | 447 | 3.575 | 15.845 | −4.897 | 1.00 | 67.56 | O | |
| ATOM | 11835 | N | VAL | C | 448 | 4.567 | 13.767 | −5.013 | 1.00 | 68.40 | N | |
| ATOM | 11836 | CA | VAL | C | 448 | 5.646 | 13.994 | −4.024 | 1.00 | 68.20 | C | |
| ATOM | 11838 | CB | VAL | C | 448 | 6.845 | 12.975 | −4.162 | 1.00 | 67.98 | C | |
| ATOM | 11840 | CG1 | VAL | C | 448 | 8.033 | 13.380 | −3.272 | 1.00 | 68.25 | C | |
| ATOM | 11844 | CG2 | VAL | C | 448 | 6.436 | 11.526 | −3.795 | 1.00 | 67.75 | C | |
| ATOM | 11848 | C | VAL | C | 448 | 6.167 | 15.436 | −4.063 | 1.00 | 68.40 | C | |
| ATOM | 11849 | O | VAL | C | 448 | 6.131 | 16.118 | −3.043 | 1.00 | 68.13 | O | |
| ATOM | 11851 | N | TYR | C | 449 | 6.591 | 15.907 | −5.236 | 1.00 | 68.99 | N | |
| ATOM | 11852 | CA | TYR | C | 449 | 7.249 | 17.211 | −5.302 | 1.00 | 69.83 | C | |
| ATOM | 11854 | CB | TYR | C | 449 | 7.898 | 17.492 | −6.659 | 1.00 | 69.69 | C | |
| ATOM | 11857 | CG | TYR | C | 449 | 8.546 | 18.863 | −6.639 | 1.00 | 69.63 | C | |
| ATOM | 11858 | CD1 | TYR | C | 449 | 9.765 | 19.070 | −5.995 | 1.00 | 69.64 | C | |
| ATOM | 11860 | CE1 | TYR | C | 449 | 10.356 | 20.342 | −5.938 | 1.00 | 70.72 | C | |
| ATOM | 11862 | CZ | TYR | C | 449 | 9.706 | 21.433 | −6.525 | 1.00 | 70.88 | C | |
| ATOM | 11863 | OH | TYR | C | 449 | 10.264 | 22.683 | −6.483 | 1.00 | 70.21 | O | |
| ATOM | 11865 | CE2 | TYR | C | 449 | 8.486 | 21.258 | −7.164 | 1.00 | 71.25 | C | |
| ATOM | 11867 | CD2 | TYR | C | 449 | 7.905 | 19.973 | −7.207 | 1.00 | 71.50 | C | |
| ATOM | 11869 | C | TYR | C | 449 | 6.370 | 18.408 | −4.855 | 1.00 | 70.32 | C | |
| ATOM | 11870 | O | TYR | C | 449 | 6.768 | 19.125 | −3.928 | 1.00 | 70.47 | O | |
| ATOM | 11872 | N | GLU | C | 450 | 5.225 | 18.630 | −5.521 | 1.00 | 70.76 | N | |
| ATOM | 11873 | CA | GLU | C | 450 | 4.280 | 19.732 | −5.165 | 1.00 | 70.83 | C | |
| ATOM | 11875 | CB | GLU | C | 450 | 2.957 | 19.618 | −5.961 | 1.00 | 71.40 | C | |
| ATOM | 11878 | CG | GLU | C | 450 | 3.006 | 20.014 | −7.440 | 1.00 | 71.50 | C | |
| ATOM | 11881 | CD | GLU | C | 450 | 1.624 | 20.038 | −8.111 | 1.00 | 71.27 | C | |
| ATOM | 11882 | OE1 | GLU | C | 450 | 0.693 | 19.345 | −7.614 | 1.00 | 70.67 | O | |
| ATOM | 11883 | OE2 | GLU | C | 450 | 1.468 | 20.751 | −9.146 | 1.00 | 71.43 | O | |
| ATOM | 11884 | C | GLU | C | 450 | 3.928 | 19.764 | −3.665 | 1.00 | 70.68 | C | |
| ATOM | 11885 | O | GLU | C | 450 | 3.851 | 20.841 | −3.056 | 1.00 | 70.17 | O | |
| ATOM | 11887 | N | LEU | C | 451 | 3.721 | 18.570 | −3.100 | 1.00 | 70.51 | N | |
| ATOM | 11888 | CA | LEU | C | 451 | 3.449 | 18.394 | −1.655 | 1.00 | 70.44 | C | |
| ATOM | 11890 | CB | LEU | C | 451 | 3.351 | 16.895 | −1.272 | 1.00 | 70.04 | C | |
| ATOM | 11893 | CG | LEU | C | 451 | 2.254 | 16.027 | −1.918 | 1.00 | 69.02 | C | |
| ATOM | 11895 | CD1 | LEU | C | 451 | 2.368 | 14.526 | −1.632 | 1.00 | 67.63 | C | |
| ATOM | 11899 | CD2 | LEU | C | 451 | 0.933 | 16.539 | −1.478 | 1.00 | 69.55 | C | |
| ATOM | 11903 | C | LEU | C | 451 | 4.470 | 19.080 | −0.738 | 1.00 | 70.53 | C | |
| ATOM | 11904 | O | LEU | C | 451 | 4.075 | 19.780 | 0.218 | 1.00 | 70.27 | O | |
| ATOM | 11906 | N | LEU | C | 452 | 5.761 | 18.886 | −1.034 | 1.00 | 70.54 | N | |
| ATOM | 11907 | CA | LEU | C | 452 | 6.832 | 19.363 | −0.142 | 1.00 | 70.96 | C | |
| ATOM | 11909 | CB | LEU | C | 452 | 8.207 | 18.767 | −0.516 | 1.00 | 71.07 | C | |
| ATOM | 11912 | CG | LEU | C | 452 | 8.374 | 17.222 | −0.627 | 1.00 | 72.07 | C | |
| ATOM | 11914 | CD1 | LEU | C | 452 | 9.673 | 16.841 | −1.365 | 1.00 | 72.21 | C | |
| ATOM | 11918 | CD2 | LEU | C | 452 | 8.326 | 16.466 | 0.699 | 1.00 | 71.84 | C | |
| ATOM | 11922 | C | LEU | C | 452 | 6.841 | 20.898 | −0.177 | 1.00 | 70.95 | C | |
| ATOM | 11923 | O | LEU | C | 452 | 6.716 | 21.548 | 0.863 | 1.00 | 70.56 | O | |
| ATOM | 11925 | N | GLU | C | 453 | 6.933 | 21.442 | −1.397 | 1.00 | 71.21 | N | |
| ATOM | 11926 | CA | GLU | C | 453 | 6.697 | 22.881 | −1.749 | 1.00 | 70.99 | C | |
| ATOM | 11928 | CB | GLU | C | 453 | 6.450 | 22.966 | −3.278 | 1.00 | 71.38 | C | |
| ATOM | 11931 | CG | GLU | C | 453 | 5.913 | 24.262 | −3.895 | 1.00 | 71.65 | C | |
| ATOM | 11934 | CD | GLU | C | 453 | 4.792 | 23.980 | −4.882 | 1.00 | 73.32 | C | |
| ATOM | 11935 | OE1 | GLU | C | 453 | 5.035 | 23.209 | −5.847 | 1.00 | 74.16 | O | |
| ATOM | 11936 | OE2 | GLU | C | 453 | 3.671 | 24.512 | −4.680 | 1.00 | 73.67 | O | |
| ATOM | 11937 | C | GLU | C | 453 | 5.564 | 23.566 | −0.987 | 1.00 | 70.18 | C | |
| ATOM | 11938 | O | GLU | C | 453 | 5.691 | 24.727 | −0.572 | 1.00 | 69.70 | O | |
| ATOM | 11940 | N | LYS | C | 454 | 4.472 | 22.831 | −0.815 | 1.00 | 69.51 | N | |
| ATOM | 11941 | CA | LYS | C | 454 | 3.303 | 23.317 | −0.100 | 1.00 | 69.10 | C | |
| ATOM | 11943 | CB | LYS | C | 454 | 2.058 | 22.969 | −0.952 | 1.00 | 69.19 | C | |
| ATOM | 11946 | CG | LYS | C | 454 | 1.065 | 24.130 | −1.167 | 1.00 | 69.12 | C | |
| ATOM | 11949 | CD | LYS | C | 454 | 1.209 | 25.098 | −2.339 | 1.00 | 68.69 | C | |
| ATOM | 11952 | CE | LYS | C | 454 | 0.105 | 26.203 | −2.160 | 1.00 | 69.15 | C | |
| ATOM | 11955 | NZ | LYS | C | 454 | 0.032 | 27.209 | −3.262 | 1.00 | 68.95 | N | |
| ATOM | 11959 | C | LYS | C | 454 | 3.246 | 22.823 | 1.401 | 1.00 | 69.04 | C | |
| ATOM | 11960 | O | LYS | C | 454 | 2.178 | 22.740 | 1.997 | 1.00 | 69.22 | O | |
| ATOM | 11962 | N | ASP | C | 455 | 4.409 | 22.519 | 1.995 | 1.00 | 68.69 | N | |
| ATOM | 11963 | CA | ASP | C | 455 | 4.631 | 22.334 | 3.451 | 1.00 | 68.14 | C | |
| ATOM | 11965 | CB | ASP | C | 455 | 3.957 | 23.472 | 4.274 | 1.00 | 68.19 | C | |
| ATOM | 11968 | CG | ASP | C | 455 | 4.923 | 24.172 | 5.225 | 1.00 | 68.33 | C | |
| ATOM | 11969 | OD1 | ASP | C | 455 | 5.255 | 23.636 | 6.292 | 1.00 | 68.67 | O | |
| ATOM | 11970 | OD2 | ASP | C | 455 | 5.404 | 25.285 | 4.984 | 1.00 | 69.69 | O | |
| ATOM | 11971 | C | ASP | C | 455 | 4.366 | 20.941 | 4.084 | 1.00 | 67.67 | C | |
| ATOM | 11972 | O | ASP | C | 455 | 4.740 | 20.740 | 5.245 | 1.00 | 67.12 | O | |
| ATOM | 11974 | N | TYR | C | 456 | 3.786 | 19.986 | 3.346 | 1.00 | 67.16 | N | |
| ATOM | 11975 | CA | TYR | C | 456 | 3.597 | 18.598 | 3.867 | 1.00 | 67.09 | C | |
| ATOM | 11977 | CB | TYR | C | 456 | 2.656 | 17.787 | 2.963 | 1.00 | 67.19 | C | |
| ATOM | 11980 | CG | TYR | C | 456 | 2.590 | 16.288 | 3.222 | 1.00 | 67.17 | C | |
| ATOM | 11981 | CD1 | TYR | C | 456 | 1.757 | 15.737 | 4.208 | 1.00 | 67.80 | C | |
| ATOM | 11983 | CE1 | TYR | C | 456 | 1.712 | 14.316 | 4.418 | 1.00 | 67.65 | C | |
| ATOM | 11985 | CZ | TYR | C | 456 | 2.503 | 13.469 | 3.627 | 1.00 | 68.06 | C | |
| ATOM | 11986 | OH | TYR | C | 456 | 2.491 | 12.097 | 3.781 | 1.00 | 68.80 | O | |
| ATOM | 11988 | CE2 | TYR | C | 456 | 3.320 | 13.998 | 2.662 | 1.00 | 67.61 | C | |
| ATOM | 11990 | CD2 | TYR | C | 456 | 3.360 | 15.404 | 2.462 | 1.00 | 68.37 | C | |
| ATOM | 11992 | C | TYR | C | 456 | 4.911 | 17.820 | 4.047 | 1.00 | 67.06 | C | |
| ATOM | 11993 | O | TYR | C | 456 | 5.767 | 17.863 | 3.171 | 1.00 | 67.59 | O | |
| ATOM | 11995 | N | ARG | C | 457 | 5.022 | 17.082 | 5.158 | 1.00 | 66.75 | N | |
| ATOM | 11996 | CA | ARG | C | 457 | 6.137 | 16.169 | 5.463 | 1.00 | 66.44 | C | |
| ATOM | 11998 | CB | ARG | C | 457 | 6.932 | 16.726 | 6.615 | 1.00 | 66.18 | C | |
| ATOM | 12001 | CG | ARG | C | 457 | 7.534 | 18.069 | 6.355 | 1.00 | 65.80 | C | |
| ATOM | 12004 | CD | ARG | C | 457 | 8.660 | 18.096 | 5.332 | 1.00 | 64.15 | C | |
| ATOM | 12007 | NE | ARG | C | 457 | 9.058 | 19.492 | 5.153 | 1.00 | 62.26 | N | |
| ATOM | 12009 | CZ | ARG | C | 457 | 8.821 | 20.270 | 4.088 | 1.00 | 60.48 | C | |
| ATOM | 12010 | NH1 | ARG | C | 457 | 8.243 | 19.823 | 2.979 | 1.00 | 58.51 | N | |
| ATOM | 12013 | NH2 | ARG | C | 457 | 9.228 | 21.538 | 4.132 | 1.00 | 61.69 | N | |
| ATOM | 12016 | C | ARG | C | 457 | 5.605 | 14.815 | 5.895 | 1.00 | 66.76 | C | |
| ATOM | 12017 | O | ARG | C | 457 | 4.389 | 14.639 | 5.979 | 1.00 | 67.72 | O | |
| ATOM | 12019 | N | MET | C | 458 | 6.487 | 13.850 | 6.163 | 1.00 | 66.57 | N | |
| ATOM | 12020 | CA | MET | C | 458 | 6.034 | 12.584 | 6.743 | 1.00 | 66.37 | C | |
| ATOM | 12022 | CB | MET | C | 458 | 7.068 | 11.479 | 6.604 | 1.00 | 66.36 | C | |
| ATOM | 12025 | CG | MET | C | 458 | 7.355 | 11.139 | 5.176 | 1.00 | 65.86 | C | |
| ATOM | 12028 | SD | MET | C | 458 | 8.109 | 9.513 | 4.975 | 1.00 | 64.58 | S | |
| ATOM | 12029 | CE | MET | C | 458 | 9.858 | 9.899 | 5.056 | 1.00 | 64.48 | C | |
| ATOM | 12033 | C | MET | C | 458 | 5.701 | 12.817 | 8.201 | 1.00 | 67.09 | C | |
| ATOM | 12034 | O | MET | C | 458 | 6.309 | 13.647 | 8.859 | 1.00 | 67.08 | O | |
| ATOM | 12036 | N | GLU | C | 459 | 4.691 | 12.110 | 8.681 | 1.00 | 68.27 | N | |
| ATOM | 12037 | CA | GLU | C | 459 | 4.270 | 12.183 | 10.090 | 1.00 | 69.09 | C | |
| ATOM | 12039 | CB | GLU | C | 459 | 2.801 | 11.718 | 10.259 | 1.00 | 69.05 | C | |
| ATOM | 12042 | CG | GLU | C | 459 | 2.512 | 10.212 | 10.330 | 1.00 | 70.70 | C | |
| ATOM | 12045 | CD | GLU | C | 459 | 2.699 | 9.472 | 9.015 | 1.00 | 73.90 | C | |
| ATOM | 12046 | OE1 | GLU | C | 459 | 3.878 | 9.284 | 8.570 | 1.00 | 77.47 | O | |
| ATOM | 12047 | OE2 | GLU | C | 459 | 1.668 | 9.061 | 8.419 | 1.00 | 73.86 | O | |
| ATOM | 12048 | C | GLU | C | 459 | 5.264 | 11.387 | 10.957 | 1.00 | 69.28 | C | |
| ATOM | 12049 | O | GLU | C | 459 | 5.896 | 10.429 | 10.468 | 1.00 | 69.90 | O | |
| ATOM | 12051 | N | ARG | C | 460 | 5.406 | 11.803 | 12.216 | 1.00 | 69.16 | N | |
| ATOM | 12052 | CA | ARG | C | 460 | 6.280 | 11.135 | 13.212 | 1.00 | 69.20 | C | |
| ATOM | 12054 | CB | ARG | C | 460 | 6.142 | 11.874 | 14.557 | 1.00 | 68.99 | C | |
| ATOM | 12057 | CG | ARG | C | 460 | 7.000 | 11.363 | 15.729 | 1.00 | 70.15 | C | |
| ATOM | 12060 | CD | ARG | C | 460 | 6.706 | 11.935 | 17.153 | 1.00 | 71.58 | C | |
| ATOM | 12063 | NE | ARG | C | 460 | 5.288 | 12.203 | 17.298 | 1.00 | 75.72 | N | |
| ATOM | 12065 | CZ | ARG | C | 460 | 4.298 | 11.269 | 17.264 | 1.00 | 79.30 | C | |
| ATOM | 12066 | NH1 | ARG | C | 460 | 4.578 | 9.946 | 17.144 | 1.00 | 81.87 | N | |
| ATOM | 12069 | NH2 | ARG | C | 460 | 3.007 | 11.667 | 17.378 | 1.00 | 76.78 | N | |
| ATOM | 12072 | C | ARG | C | 460 | 5.881 | 9.676 | 13.385 | 1.00 | 68.21 | C | |
| ATOM | 12073 | O | ARG | C | 460 | 4.777 | 9.423 | 13.820 | 1.00 | 69.06 | O | |
| ATOM | 12075 | N | PRO | C | 461 | 6.733 | 8.708 | 13.072 | 1.00 | 67.64 | N | |
| ATOM | 12076 | CA | PRO | C | 461 | 6.346 | 7.298 | 13.220 | 1.00 | 67.50 | C | |
| ATOM | 12078 | CB | PRO | C | 461 | 7.504 | 6.533 | 12.559 | 1.00 | 67.37 | C | |
| ATOM | 12081 | CG | PRO | C | 461 | 8.644 | 7.403 | 12.621 | 1.00 | 67.48 | C | |
| ATOM | 12084 | CD | PRO | C | 461 | 8.123 | 8.829 | 12.611 | 1.00 | 67.94 | C | |
| ATOM | 12087 | C | PRO | C | 461 | 6.182 | 6.896 | 14.697 | 1.00 | 67.77 | C | |
| ATOM | 12088 | O | PRO | C | 461 | 6.638 | 7.639 | 15.583 | 1.00 | 68.41 | O | |
| ATOM | 12089 | N | GLU | C | 462 | 5.518 | 5.755 | 14.941 | 1.00 | 67.31 | N | |
| ATOM | 12090 | CA | GLU | C | 462 | 5.213 | 5.279 | 16.294 | 1.00 | 66.76 | C | |
| ATOM | 12092 | CB | GLU | C | 462 | 4.457 | 3.943 | 16.269 | 1.00 | 66.38 | C | |
| ATOM | 12095 | CG | GLU | C | 462 | 3.833 | 3.562 | 17.614 | 1.00 | 66.74 | C | |
| ATOM | 12098 | CD | GLU | C | 462 | 2.325 | 3.796 | 17.750 | 1.00 | 69.26 | C | |
| ATOM | 12099 | OE1 | GLU | C | 462 | 1.587 | 3.914 | 16.728 | 1.00 | 70.12 | O | |
| ATOM | 12100 | OE2 | GLU | C | 462 | 1.840 | 3.828 | 18.921 | 1.00 | 71.30 | O | |
| ATOM | 12101 | C | GLU | C | 462 | 6.496 | 5.142 | 17.143 | 1.00 | 66.47 | C | |
| ATOM | 12102 | O | GLU | C | 462 | 7.492 | 4.587 | 16.696 | 1.00 | 66.02 | O | |
| ATOM | 12104 | N | GLY | C | 463 | 6.454 | 5.695 | 18.352 | 1.00 | 66.38 | N | |
| ATOM | 12105 | CA | GLY | C | 463 | 7.542 | 5.590 | 19.297 | 1.00 | 66.62 | C | |
| ATOM | 12108 | C | GLY | C | 463 | 8.801 | 6.338 | 18.955 | 1.00 | 66.75 | C | |
| ATOM | 12109 | O | GLY | C | 463 | 9.857 | 6.016 | 19.447 | 1.00 | 67.13 | O | |
| ATOM | 12111 | N | CYS | C | 464 | 8.691 | 7.348 | 18.115 | 1.00 | 67.75 | N | |
| ATOM | 12112 | CA | CYS | C | 464 | 9.825 | 8.169 | 17.752 | 1.00 | 67.86 | C | |
| ATOM | 12114 | CB | CYS | C | 464 | 9.634 | 8.710 | 16.355 | 1.00 | 67.42 | C | |
| ATOM | 12117 | SC | CYS | C | 464 | 11.197 | 9.330 | 15.788 | 1.00 | 69.17 | S | |
| ATOM | 12119 | C | CYS | C | 464 | 9.993 | 9.334 | 18.759 | 1.00 | 67.83 | C | |
| ATOM | 12120 | O | CYS | C | 464 | 9.058 | 10.150 | 18.903 | 1.00 | 67.66 | O | |
| ATOM | 12122 | N | PRO | C | 465 | 11.146 | 9.421 | 19.440 | 1.00 | 67.97 | N | |
| ATOM | 12123 | CA | PRO | C | 465 | 11.409 | 10.500 | 20.401 | 1.00 | 68.39 | C | |
| ATOM | 12125 | CB | PRO | C | 465 | 12.908 | 10.371 | 20.683 | 1.00 | 68.08 | C | |
| ATOM | 12128 | CG | PRO | C | 465 | 13.238 | 8.975 | 20.406 | 1.00 | 68.19 | C | |
| ATOM | 12131 | CD | PRO | C | 465 | 12.288 | 8.492 | 19.355 | 1.00 | 67.90 | C | |
| ATOM | 12134 | C | PRO | C | 465 | 11.131 | 11.930 | 19.897 | 1.00 | 69.79 | C | |
| ATOM | 12135 | O | PRO | C | 465 | 11.483 | 12.291 | 18.758 | 1.00 | 69.38 | O | |
| ATOM | 12136 | N | GLU | C | 466 | 10.462 | 12.707 | 20.755 | 1.00 | 71.40 | N | |
| ATOM | 12137 | CA | GLU | C | 466 | 10.374 | 14.174 | 20.667 | 1.00 | 71.53 | C | |
| ATOM | 12139 | CB | GLU | C | 466 | 10.379 | 14.769 | 22.103 | 1.00 | 71.76 | C | |
| ATOM | 12142 | CG | GLU | C | 466 | 10.123 | 16.278 | 22.199 | 1.00 | 72.25 | C | |
| ATOM | 12145 | CD | GLU | C | 466 | 8.677 | 16.680 | 21.877 | 1.00 | 73.86 | C | |
| ATOM | 12146 | OE1 | GLU | C | 466 | 7.817 | 16.620 | 22.810 | 1.00 | 73.77 | O | |
| ATOM | 12147 | OE2 | GLU | C | 466 | 8.404 | 17.080 | 20.708 | 1.00 | 73.17 | O | |
| ATOM | 12148 | C | GLU | C | 466 | 11.507 | 14.791 | 19.813 | 1.00 | 71.19 | C | |
| ATOM | 12149 | O | GLU | C | 466 | 11.256 | 15.308 | 18.722 | 1.00 | 71.29 | O | |
| ATOM | 12151 | N | LYS | C | 467 | 12.744 | 14.660 | 20.286 | 1.00 | 70.44 | N | |
| ATOM | 12152 | CA | LYS | C | 467 | 13.873 | 15.383 | 19.685 | 1.00 | 70.16 | C | |
| ATOM | 12154 | CB | LYS | C | 467 | 15.091 | 15.489 | 20.653 | 1.00 | 70.52 | C | |
| ATOM | 12157 | CG | LYS | C | 467 | 14.897 | 16.529 | 21.834 | 1.00 | 70.46 | C | |
| ATOM | 12160 | CD | LYS | C | 467 | 16.244 | 17.098 | 22.383 | 1.00 | 70.57 | C | |
| ATOM | 12163 | CE | LYS | C | 467 | 17.029 | 16.136 | 23.315 | 1.00 | 69.61 | C | |
| ATOM | 12166 | NZ | LYS | C | 467 | 17.144 | 16.586 | 24.724 | 1.00 | 67.96 | N | |
| ATOM | 12170 | C | LYS | C | 467 | 14.273 | 14.842 | 18.314 | 1.00 | 69.43 | C | |
| ATOM | 12171 | O | LYS | C | 467 | 14.567 | 15.639 | 17.417 | 1.00 | 68.74 | O | |
| ATOM | 12173 | N | VAL | C | 468 | 14.232 | 13.513 | 18.164 | 1.00 | 69.21 | N | |
| ATOM | 12174 | CA | VAL | C | 468 | 14.552 | 12.809 | 16.903 | 1.00 | 69.27 | C | |
| ATOM | 12176 | CB | VAL | C | 468 | 14.448 | 11.229 | 17.031 | 1.00 | 68.89 | C | |
| ATOM | 12178 | CG1 | VAL | C | 468 | 14.752 | 10.519 | 15.700 | 1.00 | 68.40 | C | |
| ATOM | 12182 | CG2 | VAL | C | 468 | 15.382 | 10.659 | 18.063 | 1.00 | 68.24 | C | |
| ATOM | 12186 | C | VAL | C | 468 | 13.648 | 13.296 | 15.758 | 1.00 | 69.33 | C | |
| ATOM | 12187 | O | VAL | C | 468 | 14.129 | 13.519 | 14.671 | 1.00 | 69.32 | O | |
| ATOM | 12189 | N | TYR | C | 469 | 12.357 | 13.468 | 16.019 | 1.00 | 70.46 | N | |
| ATOM | 12190 | CA | TYR | C | 469 | 11.410 | 13.999 | 15.027 | 1.00 | 70.99 | C | |
| ATOM | 12192 | CB | TYR | C | 469 | 9.949 | 13.802 | 15.463 | 1.00 | 71.48 | C | |
| ATOM | 12195 | CG | TYR | C | 469 | 8.939 | 14.134 | 14.360 | 1.00 | 71.27 | C | |
| ATOM | 12196 | CD1 | TYR | C | 469 | 8.949 | 13.412 | 13.161 | 1.00 | 71.53 | C | |
| ATOM | 12198 | CE1 | TYR | C | 469 | 8.045 | 13.698 | 12.121 | 1.00 | 72.46 | C | |
| ATOM | 12200 | CZ | TYR | C | 469 | 7.096 | 14.708 | 12.263 | 1.00 | 72.16 | C | |
| ATOM | 12201 | OH | TYR | C | 469 | 6.256 | 14.894 | 11.183 | 1.00 | 71.86 | O | |
| ATOM | 12203 | CE2 | TYR | C | 469 | 7.042 | 15.466 | 13.455 | 1.00 | 71.21 | C | |
| ATOM | 12205 | CD2 | TYR | C | 469 | 7.983 | 15.179 | 14.500 | 1.00 | 71.42 | C | |
| ATOM | 12207 | C | TYR | C | 469 | 11.578 | 15.467 | 14.728 | 1.00 | 71.93 | C | |
| ATOM | 12208 | O | TYR | C | 469 | 11.388 | 15.869 | 13.609 | 1.00 | 72.06 | O | |
| ATOM | 12210 | N | GLU | C | 470 | 11.852 | 16.287 | 15.734 | 1.00 | 73.68 | N | |
| ATOM | 12211 | CA | GLU | C | 470 | 12.160 | 17.720 | 15.501 | 1.00 | 74.92 | C | |
| ATOM | 12213 | CB | GLU | C | 470 | 12.503 | 18.436 | 16.801 | 1.00 | 75.20 | C | |
| ATOM | 12216 | CG | GLU | C | 470 | 11.422 | 18.556 | 17.878 | 1.00 | 76.43 | C | |
| ATOM | 12219 | CD | GLU | C | 470 | 12.030 | 18.974 | 19.245 | 1.00 | 77.35 | C | |
| ATOM | 12220 | OE1 | GLU | C | 470 | 13.208 | 19.476 | 19.281 | 1.00 | 79.25 | O | |
| ATOM | 12221 | OE2 | GLU | C | 470 | 11.341 | 18.814 | 20.295 | 1.00 | 80.28 | O | |
| ATOM | 12222 | C | GLU | C | 470 | 13.383 | 17.857 | 14.583 | 1.00 | 74.65 | C | |
| ATOM | 12223 | O | GLU | C | 470 | 13.410 | 18.735 | 13.711 | 1.00 | 74.84 | O | |
| ATOM | 12225 | N | LEU | C | 471 | 14.379 | 16.987 | 14.828 | 1.00 | 74.33 | N | |
| ATOM | 12226 | CA | LEU | C | 471 | 15.585 | 16.853 | 13.996 | 1.00 | 74.11 | C | |
| ATOM | 12228 | CB | LEU | C | 471 | 16.599 | 15.856 | 14.604 | 1.00 | 74.28 | C | |
| ATOM | 12231 | CG | LEU | C | 471 | 18.068 | 16.300 | 14.577 | 1.00 | 75.50 | C | |
| ATOM | 12233 | CD | LEU | C | 471 | 18.355 | 17.503 | 15.530 | 1.00 | 75.50 | C | |
| ATOM | 12237 | CD2 | LEU | C | 471 | 18.962 | 15.098 | 14.927 | 1.00 | 75.06 | C | |
| ATOM | 12241 | C | LEU | C | 471 | 15.224 | 16.459 | 12.557 | 1.00 | 73.85 | C | |
| ATOM | 12242 | O | LEU | C | 471 | 15.440 | 17.265 | 11.626 | 1.00 | 74.55 | O | |
| ATOM | 12244 | N | MET | C | 472 | 14.635 | 15.273 | 12.373 | 1.00 | 72.81 | N | |
| ATOM | 12245 | CA | MET | C | 472 | 14.066 | 14.865 | 11.080 | 1.00 | 72.36 | C | |
| ATOM | 12247 | CB | MET | C | 472 | 12.905 | 13.874 | 11.270 | 1.00 | 72.08 | C | |
| ATOM | 12250 | CG | MET | C | 472 | 13.287 | 12.430 | 11.308 | 1.00 | 71.91 | C | |
| ATOM | 12253 | SD | MET | C | 472 | 11.855 | 11.396 | 11.046 | 1.00 | 71.34 | S | |
| ATOM | 12254 | CE | MET | C | 472 | 12.366 | 9.958 | 11.937 | 1.00 | 72.31 | C | |
| ATOM | 12258 | C | MET | C | 472 | 13.516 | 16.052 | 10.291 | 1.00 | 72.27 | C | |
| ATOM | 12259 | O | MET | C | 472 | 13.913 | 16.293 | 9.147 | 1.00 | 73.38 | O | |
| ATOM | 12261 | N | ARG | C | 473 | 12.627 | 16.794 | 10.951 | 1.00 | 71.39 | N | |
| ATOM | 12262 | CA | ARG | C | 473 | 11.841 | 17.851 | 10.345 | 1.00 | 70.33 | C | |
| ATOM | 12264 | CB | ARG | C | 473 | 10.665 | 18.194 | 11.272 | 1.00 | 70.31 | C | |
| ATOM | 12267 | CG | ARG | C | 473 | 9.604 | 17.112 | 11.314 | 1.00 | 70.11 | C | |
| ATOM | 12270 | CD | ARG | C | 473 | 8.584 | 17.201 | 10.216 | 1.00 | 72.05 | C | |
| ATOM | 12273 | NE | ARG | C | 473 | 7.575 | 18.243 | 10.502 | 1.00 | 72.85 | N | |
| ATOM | 12275 | CZ | ARG | C | 473 | 7.559 | 19.508 | 10.030 | 1.00 | 73.03 | C | |
| ATOM | 12276 | NH1 | ARG | C | 473 | 8.492 | 19.989 | 9.191 | 1.00 | 74.44 | N | |
| ATOM | 12279 | NH2 | ARG | C | 473 | 6.580 | 20.323 | 10.412 | 1.00 | 73.45 | N | |
| ATOM | 12282 | C | ARG | C | 473 | 12.645 | 19.099 | 10.019 | 1.00 | 69.73 | C | |
| ATOM | 12283 | O | ARG | C | 473 | 12.229 | 19.869 | 9.150 | 1.00 | 68.83 | O | |
| ATOM | 12285 | N | ALA | C | 474 | 13.749 | 19.309 | 10.744 | 1.00 | 69.65 | N | |
| ATOM | 12286 | CA | ALA | C | 474 | 14.685 | 20.437 | 10.502 | 1.00 | 70.05 | C | |
| ATOM | 12288 | CB | ALA | C | 474 | 15.608 | 20.669 | 11.692 | 1.00 | 69.86 | C | |
| ATOM | 12292 | C | ALA | C | 474 | 15.523 | 20.214 | 9.248 | 1.00 | 70.15 | C | |
| ATOM | 12293 | O | ALA | C | 474 | 15.889 | 21.175 | 8.561 | 1.00 | 69.64 | O | |
| ATOM | 12295 | N | CYS | C | 475 | 15.840 | 18.943 | 8.977 | 1.00 | 70.10 | N | |
| ATOM | 12296 | CA | CYS | C | 475 | 16.419 | 18.546 | 7.685 | 1.00 | 69.60 | C | |
| ATOM | 12298 | CB | CYS | C | 475 | 16.917 | 17.102 | 7.703 | 1.00 | 69.70 | C | |
| ATOM | 12301 | SG | CYS | C | 475 | 17.878 | 16.729 | 9.138 | 1.00 | 68.18 | S | |
| ATOM | 12303 | C | CYS | C | 475 | 15.443 | 18.631 | 6.547 | 1.00 | 69.09 | C | |
| ATOM | 12304 | O | CYS | C | 475 | 15.879 | 18.750 | 5.414 | 1.00 | 70.18 | O | |
| ATOM | 12306 | N | TRP | C | 476 | 14.153 | 18.472 | 6.812 | 1.00 | 68.08 | N | |
| ATOM | 12307 | CA | TRP | C | 476 | 13.183 | 18.593 | 5.752 | 1.00 | 68.22 | C | |
| ATOM | 12309 | CB | TRP | C | 476 | 12.051 | 17.606 | 5.937 | 1.00 | 67.38 | C | |
| ATOM | 12312 | CG | TRP | C | 476 | 12.453 | 16.191 | 6.015 | 1.00 | 66.38 | C | |
| ATOM | 12313 | CD1 | TRP | C | 476 | 13.592 | 15.618 | 5.510 | 1.00 | 66.01 | C | |
| ATOM | 12315 | NE1 | TRP | C | 476 | 13.594 | 14.272 | 5.751 | 1.00 | 65.52 | N | |
| ATOM | 12317 | CE2 | TRP | C | 476 | 12.435 | 13.937 | 6.400 | 1.00 | 65.24 | C | |
| ATOM | 12318 | CD2 | TRP | C | 476 | 11.685 | 15.134 | 6.561 | 1.00 | 65.93 | C | |
| ATOM | 12319 | CE3 | TRP | C | 476 | 10.426 | 15.061 | 7.183 | 1.00 | 66.49 | C | |
| ATOM | 12321 | CZ3 | TRP | C | 476 | 9.960 | 13.792 | 7.628 | 1.00 | 66.31 | C | |
| ATOM | 12323 | CH2 | TRP | C | 476 | 10.740 | 12.625 | 7.448 | 1.00 | 65.24 | C | |
| ATOM | 12325 | CZ2 | TRP | C | 476 | 11.972 | 12.681 | 6.842 | 1.00 | 64.81 | C | |
| ATOM | 12327 | C | TRP | C | 476 | 12.654 | 20.022 | 5.580 | 1.00 | 69.63 | C | |
| ATOM | 12328 | O | TRP | C | 476 | 11.665 | 20.207 | 4.866 | 1.00 | 69.57 | O | |
| ATOM | 12330 | N | GLN | C | 477 | 13.304 | 21.035 | 6.184 | 1.00 | 70.75 | N | |
| ATOM | 12331 | CA | GLN | C | 477 | 12.910 | 22.404 | 5.933 | 1.00 | 71.84 | C | |
| ATOM | 12333 | CB | GLN | C | 477 | 13.539 | 23.419 | 6.905 | 1.00 | 72.13 | C | |
| ATOM | 12336 | CG | GLN | C | 477 | 12.784 | 23.544 | 8.248 | 1.00 | 73.41 | C | |
| ATOM | 12339 | CD | GLN | C | 477 | 11.260 | 23.795 | 8.093 | 1.00 | 73.82 | C | |
| ATOM | 12340 | OE1 | GLN | C | 477 | 10.856 | 24.741 | 7.418 | 1.00 | 73.55 | O | |
| ATOM | 12341 | NE2 | GLN | C | 477 | 10.435 | 22.925 | 8.690 | 1.00 | 72.81 | N | |
| ATOM | 12344 | C | GLN | C | 477 | 13.198 | 22.736 | 4.473 | 1.00 | 72.57 | C | |
| ATOM | 12345 | O | GLN | C | 477 | 14.325 | 22.623 | 4.023 | 1.00 | 72.58 | O | |
| ATOM | 12347 | N | TRP | C | 478 | 12.121 | 23.095 | 3.769 | 1.00 | 73.54 | N | |
| ATOM | 12348 | CA | TRP | C | 478 | 12.055 | 23.424 | 2.332 | 1.00 | 73.97 | C | |
| ATOM | 12350 | CB | TRP | C | 478 | 10.848 | 24.347 | 2.115 | 1.00 | 73.47 | C | |
| ATOM | 12353 | CG | TRP | C | 478 | 10.754 | 24.864 | 0.756 | 1.00 | 73.34 | C | |
| ATOM | 12354 | CD | TRP | C 478 | 10.940 | 26.170 | 0.331 | 1.00 | 72.98 | C | | |
| ATOM | 12356 | NE1 | TRP | C 478 | 10.756 | 26.245 | −1.027 | 1.00 | 72.16 | N | | |
| ATOM | 12358 | CE2 | TRP | C 478 | 10.469 | 24.988 | −1.506 | 1.00 | 72.89 | C | | |
| ATOM | 12359 | CD2 | TRP | C 478 | 10.465 | 24.099 | −0.398 | 1.00 | 72.99 | C | | |
| ATOM | 12360 | CE3 | TRP | C 478 | 10.177 | 22.740 | −0.620 | 1.00 | 72.89 | C | | |
| ATOM | 12362 | CZ3 | TRP | C 478 | 9.932 | 22.302 | −1.933 | 1.00 | 72.88 | C | | |
| ATOM | 12364 | CH2 | TRP | C 478 | 9.952 | 23.208 | −3.012 | 1.00 | 72.80 | C | | |
| ATOM | 12366 | CZ2 | TRP | C 478 | 10.218 | 24.550 | −2.820 | 1.00 | 73.06 | C | | |
| ATOM | 12368 | C | TRP | C | 478 | 13.276 | 24.124 | 1.754 | 1.00 | 74.71 | C | |
| ATOM | 12369 | O | TRP | C | 478 | 13.869 | 23.681 | 0.754 | 1.00 | 75.24 | O | |
| ATOM | 12371 | N | ASN | C | 479 | 13.629 | 25.228 | 2.395 | 1.00 | 75.11 | N | |
| ATOM | 12372 | CA | ASN | C | 479 | 14.726 | 26.036 | 1.937 | 1.00 | 75.48 | C | |
| ATOM | 12374 | CB | ASN | C | 479 | 14.473 | 27.488 | 2.355 | 1.00 | 75.37 | C | |
| ATOM | 12377 | CG | ASN | C | 479 | 15.418 | 28.449 | 1.731 | 1.00 | 74.29 | C | |
| ATOM | 12378 | OD1 | ASN | C | 479 | 15.844 | 28.291 | 0.590 | 1.00 | 73.05 | O | |
| ATOM | 12379 | ND2 | ASN | C | 479 | 15.758 | 29.473 | 2.481 | 1.00 | 73.74 | N | |
| ATOM | 12382 | C | ASN | C | 479 | 16.024 | 25.437 | 2.539 | 1.00 | 75.90 | C | |
| ATOM | 12383 | O | ASN | C | 479 | 16.097 | 25.337 | 3.770 | 1.00 | 75.97 | O | |
| ATOM | 12385 | N | PRO | C | 480 | 17.000 | 24.990 | 1.708 | 1.00 | 76.07 | N | |
| ATOM | 12386 | CA | PRO | C | 480 | 18.353 | 24.611 | 2.187 | 1.00 | 76.06 | C | |
| ATOM | 12388 | CB | PRO | C | 480 | 19.233 | 24.807 | 0.934 | 1.00 | 75.67 | C | |
| ATOM | 12391 | CG | PRO | C | 480 | 18.345 | 24.568 | −0.213 | 1.00 | 75.45 | C | |
| ATOM | 12394 | CD | PRO | C | 480 | 16.914 | 24.769 | 0.246 | 1.00 | 76.08 | C | |
| ATOM | 12397 | C | PRO | C | 480 | 18.926 | 25.437 | 3.341 | 1.00 | 76.36 | C | |
| ATOM | 12398 | O | PRO | C | 480 | 19.365 | 24.847 | 4.340 | 1.00 | 75.80 | O | |
| ATOM | 12399 | N | SER | C | 481 | 18.869 | 26.777 | 3.199 | 1.00 | 76.68 | N | |
| ATOM | 12400 | CA | SER | C | 481 | 19.385 | 27.743 | 4.191 | 1.00 | 77.15 | C | |
| ATOM | 12402 | CB | SER | C | 481 | 18.914 | 29.166 | 3.891 | 1.00 | 77.22 | C | |
| ATOM | 12405 | OG | SER | C | 481 | 19.306 | 29.598 | 2.605 | 1.00 | 77.53 | O | |
| ATOM | 12407 | C | SER | C | 481 | 18.992 | 27.420 | 5.632 | 1.00 | 77.64 | C | |
| ATOM | 12408 | O | SER | C | 481 | 19.786 | 27.634 | 6.551 | 1.00 | 77.97 | 0 | |
| ATOM | 12410 | N | ASP | C | 482 | 17.767 | 26.911 | 5.806 | 1.00 | 78.01 | N | |
| ATOM | 12411 | CA | ASP | C | 482 | 17.201 | 26.556 | 7.128 | 1.00 | 78.31 | C | |
| ATOM | 12413 | CB | ASP | C | 482 | 15.763 | 25.992 | 7.004 | 1.00 | 78.82 | C | |
| ATOM | 12416 | CG | ASP | C | 482 | 14.806 | 26.925 | 6.261 | 1.00 | 80.70 | C | |
| ATOM | 12417 | OD1 | ASP | C | 482 | 14.795 | 28.169 | 6.514 | 1.00 | 79.60 | O | |
| ATOM | 12418 | OD2 | ASP | C | 482 | 14.036 | 26.458 | 5.385 | 1.00 | 84.01 | 0 | |
| ATOM | 12419 | C | ASP | C | 482 | 18.013 | 25.490 | 7.858 | 1.00 | 77.92 | C | |
| ATOM | 12420 | O | ASP | C | 482 | 18.431 | 25.692 | 8.994 | 1.00 | 78.44 | 0 | |
| ATOM | 12422 | N | ARG | C | 483 | 18.263 | 24.391 | 7.152 | 1.00 | 77.04 | N | |
| ATOM | 12423 | CA | ARG | C | 483 | 18.598 | 23.096 | 7.739 | 1.00 | 76.48 | C | |
| ATOM | 12425 | CB | ARG | C | 483 | 18.844 | 22.077 | 6.613 | 1.00 | 76.55 | C | |
| ATOM | 12428 | CG | ARG | C | 483 | 17.615 | 21.728 | 5.743 | 1.00 | 76.73 | C | |
| ATOM | 12431 | CD | ARG | C | 483 | 17.972 | 21.022 | 4.423 | 1.00 | 77.29 | C | |
| ATOM | 12434 | NE | ARG | C | 483 | 16.899 | 21.219 | 3.455 | 1.00 | 78.39 | N | |
| ATOM | 12436 | CZ | ARG | C | 483 | 17.019 | 21.221 | 2.116 | 1.00 | 79.19 | C | |
| ATOM | 12437 | NH1 | ARG | C | 483 | 18.171 | 20.962 | 1.501 | 1.00 | 78.83 | N | |
| ATOM | 12440 | NH2 | ARG | C | 483 | 15.934 | 21.472 | 1.376 | 1.00 | 79.47 | N | |
| ATOM | 12443 | C | ARG | C | 483 | 19.816 | 23.131 | 8.649 | 1.00 | 75.73 | C | |
| ATOM | 12444 | O | ARG | C | 483 | 20.659 | 23.998 | 8.488 | 1.00 | 75.54 | O | |
| ATOM | 12446 | N | PRO | C | 484 | 19.940 | 22.183 | 9.579 | 1.00 | 75.37 | N | |
| ATOM | 12447 | CA | PRO | C | 484 | 21.086 | 22.173 | 10.472 | 1.00 | 75.01 | C | |
| ATOM | 12449 | CB | PRO | C | 484 | 20.677 | 21.181 | 11.564 | 1.00 | 75.00 | C | |
| ATOM | 12452 | CG | PRO | C | 484 | 19.846 | 20.200 | 10.856 | 1.00 | 75.41 | C | |
| ATOM | 12455 | CD | PRO | C | 484 | 19.065 | 21.021 | 9.845 | 1.00 | 75.69 | C | |
| ATOM | 12458 | C | PRO | C | 484 | 22.307 | 21.663 | 9.736 | 1.00 | 74.47 | C | |
| ATOM | 12459 | O | PRO | C | 484 | 22.203 | 21.202 | 8.610 | 1.00 | 74.36 | O | |
| ATOM | 12460 | N | SER | C | 485 | 23.449 | 21.783 | 10.389 | 1.00 | 74.07 | N | |
| ATOM | 12461 | CA | SER | C | 485 | 24.686 | 21.199 | 9.930 | 1.00 | 73.51 | C | |
| ATOM | 12463 | CB | SER | C | 485 | 25.881 | 22.030 | 10.419 | 1.00 | 73.50 | C | |
| ATOM | 12466 | OG | SER | C | 485 | 25.722 | 22.407 | 11.770 | 1.00 | 72.87 | O | |
| ATOM | 12468 | C | SER | C | 485 | 24.746 | 19.802 | 10.496 | 1.00 | 73.26 | C | |
| ATOM | 12469 | O | SER | C | 485 | 24.110 | 19.507 | 11.502 | 1.00 | 72.75 | O | |
| ATOM | 12471 | N | PHE | C | 486 | 25.526 | 18.950 | 9.839 | 1.00 | 73.47 | N | |
| ATOM | 12472 | CA | PHE | C | 486 | 25.917 | 17.650 | 10.392 | 1.00 | 73.80 | C | |
| ATOM | 12474 | CB | PHE | C | 486 | 26.637 | 16.777 | 9.360 | 1.00 | 73.84 | C | |
| ATOM | 12477 | CG | PHE | C | 486 | 25.704 | 16.128 | 8.370 | 1.00 | 74.33 | C | |
| ATOM | 12478 | CD1 | PHE | C | 486 | 24.739 | 15.207 | 8.804 | 1.00 | 74.58 | C | |
| ATOM | 12480 | CE1 | PHE | C | 486 | 23.874 | 14.586 | 7.900 | 1.00 | 73.37 | C | |
| ATOM | 12482 | CZ | PHE | C | 486 | 23.957 | 14.870 | 6.565 | 1.00 | 72.96 | C | |
| ATOM | 12484 | CE2 | PHE | C | 486 | 24.915 | 15.779 | 6.109 | 1.00 | 73.34 | C | |
| ATOM | 12486 | CD2 | PHE | C | 486 | 25.787 | 16.405 | 7.011 | 1.00 | 73.39 | C | |
| ATOM | 12488 | C | PHE | C | 486 | 26.776 | 17.767 | 11.631 | 1.00 | 74.22 | C | |
| ATOM | 12489 | O | PHE | C | 486 | 26.900 | 16.777 | 12.371 | 1.00 | 74.51 | O | |
| ATOM | 12491 | N | ALA | C | 487 | 27.404 | 18.943 | 11.809 | 1.00 | 74.41 | N | |
| ATOM | 12492 | CA | ALA | C | 487 | 27.946 | 19.402 | 13.099 | 1.00 | 74.38 | C | |
| ATOM | 12494 | CB | ALA | C | 487 | 28.663 | 20.742 | 12.910 | 1.00 | 74.38 | C | |
| ATOM | 12498 | C | ALA | C | 487 | 26.884 | 19.511 | 14.213 | 1.00 | 74.17 | C | |
| ATOM | 12499 | O | ALA | C | 487 | 27.055 | 18.956 | 15.294 | 1.00 | 73.60 | O | |
| ATOM | 12501 | N | GLU | C | 488 | 25.796 | 20.224 | 13.934 | 1.00 | 74.48 | N | |
| ATOM | 12502 | CA | GLU | C | 488 | 24.611 | 20.232 | 14.816 | 1.00 | 75.02 | C | |
| ATOM | 12504 | CB | GLU | C | 488 | 23.537 | 21.228 | 14.299 | 1.00 | 75.18 | C | |
| ATOM | 12507 | CG | GLU | C | 488 | 23.884 | 22.709 | 14.492 | 1.00 | 75.87 | C | |
| ATOM | 12510 | CD | GLU | C | 488 | 23.055 | 23.678 | 13.633 | 1.00 | 75.88 | C | |
| ATOM | 12511 | OE1 | GLU | C | 488 | 21.796 | 23.548 | 13.591 | 1.00 | 75.64 | O | |
| ATOM | 12512 | OE2 | GLU | C | 488 | 23.674 | 24.595 | 13.016 | 1.00 | 76.33 | O | |
| ATOM | 12513 | C | GLU | C | 488 | 23.981 | 18.825 | 15.021 | 1.00 | 75.06 | C | |
| ATOM | 12514 | O | GLU | C | 488 | 23.632 | 18.458 | 16.146 | 1.00 | 74.98 | O | |
| ATOM | 12516 | N | ILE | C | 489 | 23.862 | 18.046 | 13.944 | 1.00 | 75.01 | N | |
| ATOM | 12517 | CA | ILE | C | 489 | 23.168 | 16.743 | 13.985 | 1.00 | 75.07 | C | |
| ATOM | 12519 | CB | ILE | C | 489 | 22.870 | 16.224 | 12.523 | 1.00 | 75.14 | C | |
| ATOM | 12521 | CG1 | ILE | C | 489 | 21.895 | 17.158 | 11.784 | 1.00 | 74.44 | C | |
| ATOM | 12524 | CD1 | ILE | C | 489 | 22.041 | 17.116 | 10.282 | 1.00 | 74.50 | C | |
| ATOM | 12528 | CG2 | ILE | C | 489 | 22.303 | 14.788 | 12.512 | 1.00 | 75.22 | C | |
| ATOM | 12532 | C | ILE | C | 489 | 23.936 | 15.704 | 14.833 | 1.00 | 75.35 | C | |
| ATOM | 12533 | O | ILE | C | 489 | 23.339 | 14.968 | 15.598 | 1.00 | 75.25 | O | |
| ATOM | 12535 | N | HIS | C | 490 | 25.250 | 15.647 | 14.687 | 1.00 | 75.80 | N | |
| ATOM | 12536 | CA | HIS | C | 490 | 26.088 | 14.850 | 15.583 | 1.00 | 76.30 | C | |
| ATOM | 12538 | CB | HIS | C | 490 | 27.563 | 14.905 | 15.147 | 1.00 | 76.62 | C | |
| ATOM | 12541 | CG | HIS | C | 490 | 28.450 | 13.918 | 15.855 | 1.00 | 77.40 | C | |
| ATOM | 12542 | ND1 | HIS | C | 490 | 28.200 | 12.559 | 15.865 | 1.00 | 78.16 | N | |
| ATOM | 12544 | CE1 | HIS | C | 490 | 29.142 | 11.946 | 16.562 | 1.00 | 77.55 | C | |
| ATOM | 12546 | NE2 | HIS | C | 490 | 30.006 | 12.852 | 16.982 | 1.00 | 77.45 | N | |
| ATOM | 12548 | CD2 | HIS | C | 490 | 29.596 | 14.093 | 16.557 | 1.00 | 77.25 | C | |
| ATOM | 12550 | C | HIS | C | 490 | 25.976 | 15.328 | 17.033 | 1.00 | 76.29 | C | |
| ATOM | 12551 | O | HIS | C | 490 | 26.026 | 14.510 | 17.949 | 1.00 | 76.24 | O | |
| ATOM | 12553 | N | GLN | C | 491 | 25.831 | 16.641 | 17.232 | 1.00 | 76.39 | N | |
| ATOM | 12554 | CA | GLN | C | 491 | 25.763 | 17.228 | 18.586 | 1.00 | 76.44 | C | |
| ATOM | 12556 | CB | GLN | C | 491 | 25.732 | 18.773 | 18.542 | 1.00 | 76.44 | C | |
| ATOM | 12559 | CG | GLN | C | 491 | 25.997 | 19.472 | 19.858 | 1.00 | 76.35 | C | |
| ATOM | 12562 | CD | GLN | C | 491 | 27.330 | 19.112 | 20.475 | 1.00 | 76.73 | C | |
| ATOM | 12563 | OE1 | GLN | C | 491 | 27.474 | 19.155 | 21.689 | 1.00 | 77.27 | O | |
| ATOM | 12564 | NE2 | GLN | C | 491 | 28.305 | 18.751 | 19.646 | 1.00 | 77.64 | N | |
| ATOM | 12567 | C | GLN | C | 491 | 24.556 | 16.718 | 19.354 | 1.00 | 76.13 | C | |
| ATOM | 12568 | O | GLN | C | 491 | 24.700 | 16.209 | 20.460 | 1.00 | 76.22 | O | |
| ATOM | 12570 | N | ALA | C | 492 | 23.388 | 16.853 | 18.738 | 1.00 | 75.69 | N | |
| ATOM | 12571 | CA | ALA | C | 492 | 22.165 | 16.288 | 19.250 | 1.00 | 75.71 | C | |
| ATOM | 12573 | CB | ALA | C | 492 | 21.039 | 16.526 | 18.278 | 1.00 | 75.71 | C | |
| ATOM | 12577 | C | ALA | C | 492 | 22.322 | 14.795 | 19.507 | 1.00 | 76.10 | C | |
| ATOM | 12578 | O | ALA | C | 492 | 22.096 | 14.329 | 20.630 | 1.00 | 76.68 | O | |
| ATOM | 12580 | N | PHE | C | 493 | 22.746 | 14.061 | 18.486 | 1.00 | 76.26 | N | |
| ATOM | 12581 | CA | PHE | C | 493 | 22.816 | 12.596 | 18.566 | 1.00 | 76.75 | C | |
| ATOM | 12583 | CB | PHE | C | 493 | 22.950 | 11.929 | 17.172 | 1.00 | 76.94 | C | |
| ATOM | 12586 | CG | PHE | C | 493 | 21.636 | 11.535 | 16.567 | 1.00 | 76.69 | C | |
| ATOM | 12587 | CD1 | PHE | C | 493 | 20.961 | 10.400 | 17.042 | 1.00 | 78.13 | C | |
| ATOM | 12589 | CE1 | PHE | C | 493 | 19.711 | 10.024 | 16.520 | 1.00 | 77.96 | C | |
| ATOM | 12591 | CZ | PHE | C | 493 | 19.144 | 10.772 | 15.515 | 1.00 | 77.10 | C | |
| ATOM | 12593 | CE2 | PHE | C | 493 | 19.810 | 11.913 | 15.040 | 1.00 | 77.24 | C | |
| ATOM | 12595 | CD2 | PHE | C | 493 | 21.049 | 12.289 | 15.562 | 1.00 | 76.21 | C | |
| ATOM | 12597 | C | PHE | C | 493 | 23.880 | 12.049 | 19.506 | 1.00 | 76.93 | C | |
| ATOM | 12598 | O | PHE | C | 493 | 23.703 | 10.939 | 19.989 | 1.00 | 76.69 | O | |
| ATOM | 12600 | N | GLU | C | 494 | 24.973 | 12.790 | 19.745 | 1.00 | 77.61 | N | |
| ATOM | 12601 | CA | GLU | C | 494 | 25.955 | 12.420 | 20.801 | 1.00 | 77.86 | C | |
| ATOM | 12603 | CB | GLU | C | 494 | 27.312 | 13.194 | 20.695 | 1.00 | 77.92 | C | |
| ATOM | 12606 | CG | GLU | C | 494 | 28.551 | 12.383 | 21.147 | 1.00 | 78.10 | C | |
| ATOM | 12609 | CD | GLU | C | 494 | 29.859 | 13.185 | 21.333 | 1.00 | 78.45 | C | |
| ATOM | 12610 | OE1 | GLU | C | 494 | 29.861 | 14.231 | 22.018 | 1.00 | 79.49 | O | |
| ATOM | 12611 | OE2 | GLU | C | 494 | 30.924 | 12.752 | 20.831 | 1.00 | 78.61 | O | |
| ATOM | 12612 | C | GLU | C | 494 | 25.261 | 12.606 | 22.175 | 1.00 | 77.87 | C | |
| ATOM | 12613 | O | GLU | C | 494 | 25.228 | 11.666 | 22.977 | 1.00 | 77.35 | O | |
| ATOM | 12615 | N | THR | C | 495 | 24.658 | 13.787 | 22.388 | 1.00 | 78.20 | N | |
| ATOM | 12616 | CA | THR | C | 495 | 23.856 | 14.098 | 23.593 | 1.00 | 78.66 | C | |
| ATOM | 12618 | CB | THR | C | 495 | 23.254 | 15.537 | 23.530 | 1.00 | 78.21 | C | |
| ATOM | 12620 | OG1 | THR | C | 495 | 24.249 | 16.469 | 23.101 | 1.00 | 77.25 | O | |
| ATOM | 12622 | CG2 | THR | C | 495 | 22.871 | 16.045 | 24.907 | 1.00 | 77.26 | C | |
| ATOM | 12626 | C | THR | C | 495 | 22.747 | 13.060 | 23.861 | 1.00 | 79.40 | C | |
| ATOM | 12627 | O | THR | C | 495 | 22.860 | 12.310 | 24.836 | 1.00 | 79.36 | O | |
| ATOM | 12629 | N | MET | C | 496 | 21.733 | 12.980 | 22.983 | 1.00 | 80.22 | N | |
| ATOM | 12630 | CA | MET | C | 496 | 20.602 | 12.025 | 23.134 | 1.00 | 81.03 | C | |
| ATOM | 12632 | CB | MET | C | 496 | 19.734 | 11.959 | 21.877 | 1.00 | 81.25 | C | |
| ATOM | 12635 | CG | MET | C | 496 | 18.940 | 13.203 | 21.537 | 1.00 | 82.06 | C | |
| ATOM | 12638 | SD | MET | C | 496 | 17.636 | 12.796 | 20.286 | 1.00 | 82.93 | S | |
| ATOM | 12639 | CE | MET | C | 496 | 18.091 | 13.928 | 18.988 | 1.00 | 82.16 | C | |
| ATOM | 12643 | C | MET | C | 496 | 21.005 | 10.583 | 23.441 | 1.00 | 81.63 | C | |
| ATOM | 12644 | O | MET | C | 496 | 20.317 | 9.902 | 24.181 | 1.00 | 81.71 | O | |
| ATOM | 12646 | N | PHE | C | 497 | 22.102 | 10.133 | 22.843 | 1.00 | 82.65 | N | |
| ATOM | 12647 | CA | PHE | C | 497 | 22.613 | 8.762 | 23.010 | 1.00 | 83.43 | C | |
| ATOM | 12649 | CB | PHE | C | 497 | 23.761 | 8.518 | 22.025 | 1.00 | 83.60 | C | |
| ATOM | 12652 | CG | PHE | C | 497 | 24.385 | 7.164 | 22.131 | 1.00 | 83.70 | C | |
| ATOM | 12653 | CD1 | PHE | C | 497 | 23.603 | 6.015 | 21.985 | 1.00 | 83.53 | C | |
| ATOM | 12655 | CE1 | PHE | C | 497 | 24.180 | 4.748 | 22.055 | 1.00 | 83.37 | C | |
| ATOM | 12657 | CZ | PHE | C | 497 | 25.554 | 4.622 | 22.248 | 1.00 | 83.54 | C | |
| ATOM | 12659 | CE2 | PHE | C | 497 | 26.355 | 5.773 | 22.397 | 1.00 | 83.62 | C | |
| ATOM | 12661 | CD2 | PHE | C | 497 | 25.764 | 7.032 | 22.336 | 1.00 | 84.04 | C | |
| ATOM | 12663 | C | PHE | C | 497 | 23.063 | 8.396 | 24.432 | 1.00 | 83.69 | C | |
| ATOM | 12664 | O | PHE | C | 497 | 22.361 | 7.635 | 25.092 | 1.00 | 83.94 | O | |
| ATOM | 12666 | N | GLN | C | 498 | 24.213 | 8.917 | 24.890 | 1.00 | 83.93 | N | |
| ATOM | 12667 | CA | GLN | C | 498 | 24.795 | 8.523 | 26.211 | 1.00 | 83.93 | C | |
| ATOM | 12669 | CB | GLN | C | 498 | 26.308 | 8.849 | 26.298 | 1.00 | 84.04 | C | |
| ATOM | 12672 | CG | GLN | C | 498 | 26.682 | 10.349 | 26.294 | 1.00 | 83.90 | C | |
| ATOM | 12675 | CD | GLN | C | 498 | 28.121 | 10.635 | 25.841 | 1.00 | 83.20 | C | |
| ATOM | 12676 | OE1 | GLN | C | 498 | 28.516 | 10.274 | 24.737 | 1.00 | 80.32 | O | |
| ATOM | 12677 | NE2 | GLN | C | 498 | 28.886 | 11.309 | 26.689 | 1.00 | 82.58 | N | |
| ATOM | 12680 | C | GLN | C | 498 | 24.009 | 9.016 | 27.459 | 1.00 | 84.24 | C | |
| ATOM | 12681 | O | GLN | C | 498 | 24.255 | 8.538 | 28.575 | 1.00 | 84.05 | O | |
| ATOM | 12683 | N | GLN | C | 499 | 23.082 | 9.963 | 27.262 | 1.00 | 84.51 | N | |
| ATOM | 12684 | CA | GLN | C | 499 | 21.974 | 10.190 | 28.200 | 1.00 | 84.91 | C | |
| ATOM | 12686 | CB | GLN | C | 499 | 21.136 | 11.414 | 27.819 | 1.00 | 84.75 | C | |
| ATOM | 12689 | CG | GLU | C | 499 | 21.851 | 12.736 | 28.050 | 1.00 | 84.75 | C | |
| ATOM | 12692 | CD | GLU | C | 499 | 20.996 | 13.945 | 27.686 | 1.00 | 84.95 | C | |
| ATOM | 12693 | OE1 | GLU | C | 499 | 20.417 | 13.974 | 26.573 | 1.00 | 86.01 | O | |
| ATOM | 12694 | OE2 | GLU | C | 499 | 20.907 | 14.884 | 28.504 | 1.00 | 83.89 | O | |
| ATOM | 12695 | C | GLU | C | 499 | 21.080 | 8.954 | 28.247 | 1.00 | 85.30 | C | |
| ATOM | 12696 | O | GLU | C | 499 | 20.837 | 8.413 | 29.326 | 1.00 | 85.69 | O | |
| ATOM | 12698 | N | SER | C | 500 | 20.615 | 8.514 | 27.078 | 1.00 | 85.74 | N | |
| ATOM | 12699 | CA | SER | C | 500 | 19.830 | 7.265 | 26.939 | 1.00 | 86.35 | C | |
| ATOM | 12701 | CB | SER | C | 500 | 19.173 | 7.153 | 25.534 | 1.00 | 86.69 | C | |
| ATOM | 12704 | OG | SER | C | 500 | 18.794 | 5.823 | 25.197 | 1.00 | 87.87 | O | |
| ATOM | 12706 | C | SER | C | 500 | 20.697 | 6.044 | 27.233 | 1.00 | 86.23 | C | |
| ATOM | 12707 | O | SER | C | 500 | 20.791 | 5.621 | 28.378 | 1.00 | 86.31 | O | |
| ATOM | 12709 | O27 | INH | I | 501 | 50.633 | 14.353 | 58.105 | 1.00 | 47.28 | O | |
| ATOM | 12710 | C26 | INH | I | 501 | 50.065 | 15.076 | 58.915 | 1.00 | 47.55 | C | |
| ATOM | 12711 | N24 | INH | I | 501 | 48.744 | 15.160 | 59.089 | 1.00 | 47.40 | N | |
| ATOM | 12713 | C21 | INH | I | 501 | 48.278 | 16.088 | 60.124 | 1.00 | 47.21 | C | |
| ATOM | 12716 | C9 | INH | I | 501 | 49.607 | 16.598 | 60.592 | 1.00 | 47.20 | C | |
| ATOM | 12717 | C10 | INH | I | 501 | 49.830 | 17.559 | 61.607 | 1.00 | 47.36 | C | |
| ATOM | 12718 | C2 | INH | I | 501 | 51.202 | 17.896 | 61.884 | 1.00 | 47.25 | C | |
| ATOM | 12719 | C8 | INH | I | 501 | 50.655 | 16.010 | 59.876 | 1.00 | 47.56 | C | |
| ATOM | 12720 | C7 | INH | I | 501 | 52.013 | 16.352 | 60.146 | 1.00 | 47.51 | C | |
| ATOM | 12721 | C3 | INH | I | 501 | 52.224 | 17.321 | 61.199 | 1.00 | 47.43 | C | |
| ATOM | 12722 | C6 | INH | I | 501 | 53.330 | 15.986 | 59.668 | 1.00 | 47.48 | C | |
| ATOM | 12723 | C28 | INH | I | 501 | 53.690 | 15.014 | 58.565 | 1.00 | 47.38 | C | |
| ATOM | 12726 | C31 | INH | I | 501 | 55.197 | 14.809 | 58.362 | 1.00 | 46.70 | C | |
| ATOM | 12729 | C34 | INH | I | 501 | 56.003 | 16.072 | 58.728 | 1.00 | 46.96 | C | |
| ATOM | 12732 | C37 | INH | I | 501 | 55.723 | 16.581 | 60.155 | 1.00 | 47.49 | C | |
| ATOM | 12735 | C5 | INH | I | 501 | 54.233 | 16.697 | 60.372 | 1.00 | 47.31 | C | |
| ATOM | 12736 | N4 | INH | I | 501 | 53.599 | 17.5O5 | 61.275 | 1.00 | 47.53 | N | |
| ATOM | 12737 | C40 | INH | I | 501 | 54.292 | 18.373 | 62.200 | 1.00 | 47.38 | C | |
| ATOM | 12739 | C48 | INH | I | 501 | 54.785 | 19.665 | 61.519 | 1.00 | 46.93 | C | |
| ATOM | 12742 | C46 | INH | I | 501 | 54.362 | 20.848 | 62.393 | 1.00 | 47.05 | C | |
| ATOM | 12744 | NS1 | INH | I | 501 | 54.763 | 22.190 | 61.909 | 1.00 | 48.18 | N | |
| ATOM | 12746 | C53 | INH | I | 501 | 56.125 | 22.499 | 61.406 | 1.00 | 48.59 | C | |
| ATOM | 127S0 | C44 | INH | I | 501 | 52.842 | 20.770 | 62.664 | 1.00 | 47.40 | C | |
| ATOM | 12752 | O57 | INH | I | 501 | 51.888 | 21.195 | 61.632 | 1.00 | 45.70 | O | |
| ATOM | 12753 | C58 | INH | I | 501 | 51.800 | 20.708 | 60.301 | 1.00 | 42.85 | C | |
| ATOM | 12757 | C43 | INH | I | 501 | 52.442 | 19.448 | 63.439 | 1.00 | 47.56 | C | |
| ATOM | 12758 | O42 | INH | I | 501 | 53.515 | 18.461 | 63.411 | 1.00 | 45.24 | O | |
| ATOM | 12759 | C62 | INH | I | 501 | 52.390 | 19.715 | 64.974 | 1.00 | 47.79 | C | |
| ATOM | 12763 | N1 | INH | I | 501 | 51.174 | 18.860 | 62.912 | 1.00 | 47.89 | N | |
| ATOM | 12764 | C12 | INH | I | 501 | 49.815 | 19.129 | 63.256 | 1.00 | 47.31 | C | |
| ATOM | 12765 | C11 | INH | I | 501 | 48.994 | 18.348 | 62.500 | 1.00 | 47.60 | C | |
| ATOM | 12766 | C19 | INH | I | 501 | 47.577 | 18.361 | 62.613 | 1.00 | 47.54 | C | |
| ATOM | 12768 | C17 | INH | I | 501 | 46.986 | 19.219 | 63.567 | 1.00 | 47.59 | C | |
| ATOM | 12770 | C15 | INH | I | 501 | 47.814 | 20.022 | 64.375 | 1.00 | 47.42 | C | |
| ATOM | 12772 | C13 | INH | I | 501 | 49.219 | 19.978 | 64.214 | 1.00 | 47.09 | C | |
| ATOM | 12774 | O27 | INH | J | 501 | 31.430 | 6.054 | 23.367 | 1.00 | 65.83 | O | |
| ATOM | 12775 | C26 | INH | J | 501 | 30.619 | 5.142 | 23.223 | 1.00 | 65.94 | C | |
| ATOM | 12776 | N24 | INH | J | 501 | 30.418 | 4.380 | 22.131 | 1.00 | 65.75 | N | |
| ATOM | 12778 | C21 | INH | J | 501 | 29.363 | 3.368 | 22.235 | 1.00 | 65.64 | C | |
| ATOM | 12781 | C9 | INH | J | 501 | 28.946 | 3.650 | 23.658 | 1.00 | 65.97 | C | |
| ATOM | 12782 | C10 | INH | J | 501 | 27.927 | 3.002 | 24.392 | 1.00 | 65.87 | C | |
| ATOM | 12783 | C2 | INH | J | 501 | 27.699 | 3.458 | 25.741 | 1.00 | 65.78 | C | |
| ATOM | 12784 | C8 | INH | J | 501 | 29.680 | 4.697 | 24.228 | 1.00 | 66.06 | C | |
| ATOM | 1278S | C7 | INH | J | 501 | 29.473 | 5.161 | 25.562 | 1.00 | 65.85 | C | |
| ATOM | 12786 | C3 | INH | J | 501 | 28.431 | 4.475 | 26.290 | 1.00 | 65.75 | C | |
| ATOM | 12787 | C6 | INH | J | 501 | 30.017 | 6.167 | 26.456 | 1.00 | 65.73 | C | |
| ATOM | 12788 | C28 | INH | J | 501 | 31.121 | 7.174 | 26.228 | 1.00 | 65.76 | C | |
| ATOM | 12791 | C31 | INH | J | 501 | 31.413 | 8.124 | 27.424 | 1.00 | 66.61 | C | |
| ATOM | 12794 | C34 | INH | J | 501 | 30.214 | 8.311 | 28.377 | 1.00 | 66.11 | C | |
| ATOM | 12797 | C37 | INH | J | 501 | 29.605 | 6.968 | 28.813 | 1.00 | 65.84 | C | |
| ATOM | 12800 | C5 | INH | J | 501 | 29.326 | 6.082 | 27.612 | 1.00 | 65.78 | C | |
| ATOM | 12801 | N4 | INH | J | 501 | 28.370 | 5.096 | 27.533 | 1.00 | 65.85 | N | |
| ATOM | 12802 | C40 | INH | J | 501 | 27.535 | 4.687 | 28.654 | 1.00 | 65.93 | C | |
| ATOM | 12804 | C48 | INH | J | 501 | 26.385 | 5.605 | 29.134 | 1.00 | 66.61 | C | |
| ATOM | 12807 | C46 | INH | J | 501 | 25.090 | 4.773 | 29.114 | 1.00 | 68.12 | C | |
| ATOM | 12809 | N51 | INH | J | 501 | 23.828 | 5.541 | 29.276 | 1.00 | 70.21 | N | |
| ATOM | 12811 | C53 | INH | J | 501 | 23.236 | 5.886 | 30.583 | 1.00 | 70.99 | C | |
| ATOM | 12815 | C44 | INH | J | 501 | 25.082 | 4.032 | 27.776 | 1.00 | 67.83 | C | |
| ATOM | 12817 | O57 | INH | J | 501 | 25.080 | 5.052 | 26.758 | 1.00 | 69.18 | O | |
| ATOM | 12818 | C58 | INH | J | 501 | 24.560 | 4.758 | 25.465 | 1.00 | 71.16 | C | |
| ATOM | 12822 | C43 | INH | J | 501 | 26.159 | 2.889 | 27.624 | 1.00 | 66.11 | C | |
| ATOM | 12823 | O42 | INH | J | 501 | 27.265 | 3.274 | 28.459 | 1.00 | 64.71 | O | |
| ATOM | 12824 | C62 | INH | J | 501 | 25.767 | 1.588 | 28.354 | 1.00 | 66.69 | C | |
| ATOM | 12828 | N1 | INH | J | 501 | 26.659 | 2.665 | 26.247 | 1.00 | 65.99 | N | |
| ATOM | 12829 | C12 | INH | J | 501 | 26.251 | 1.760 | 25.235 | 1.00 | 65.93 | C | |
| ATOM | 12830 | C11 | INH | J | 501 | 27.011 | 1.935 | 24.122 | 1.00 | 65.86 | C | |
| ATOM | 12831 | C19 | INH | J | 501 | 26.855 | 1.173 | 22.927 | 1.00 | 66.04 | C | |
| ATOM | 12833 | C17 | INH | J | 501 | 25.863 | 0.151 | 22.866 | 1.00 | 65.95 | C | |
| ATOM | 12835 | C15 | INH | J | 501 | 25.063 | −0.078 | 24.014 | 1.00 | 66.05 | C | |
| ATOM | 12837 | C13 | INH | J | 501 | 25.255 | 0.728 | 25.186 | 1.00 | 66.17 | C | |
| ATOM | 12839 | O27 | INH | K | 501 | 43.601 | 2.446 | 9.301 | 1.00 | 77.97 | O | |
| ATOM | 12840 | C26 | INH | K | 501 | 43.897 | 1.265 | 9.367 | 1.00 | 78.00 | C | |
| ATOM | 12841 | N24 | INH | K | 501 | 43.794 | 0.503 | 10.446 | 1.00 | 77.96 | N | |
| ATOM | 12843 | C21 | INH | K | 501 | 44.213 | −0.892 | 10.274 | 1.00 | 78.01 | C | |
| ATOM | 12846 | C9 | INH | K | 501 | 44.615 | −0.853 | 8.822 | 1.00 | 78.18 | C | |
| ATOM | 12847 | C10 | INH | K | 501 | 45.134 | −1.918 | 8.017 | 1.00 | 78.28 | C | |
| ATOM | 12848 | C2 | INH | K | 501 | 45.446 | −1.616 | 6.640 | 1.00 | 78.12 | C | |
| ATOM | 12849 | C8 | INH | K | 501 | 44.417 | 0.425 | 8.307 | 1.00 | 78.13 | C | |
| ATOM | 12850 | C7 | INH | K | 501 | 44.728 | 0.735 | 6.960 | 1.00 | 78.08 | C | |
| ATOM | 12851 | C3 | INH | K | 501 | 45.250 | −0.354 | 6.160 | 1.00 | 77.92 | C | |
| ATOM | 128S2 | C6 | INH | K | 501 | 44.663 | 1.901 | 6.098 | 1.00 | 78.20 | C | |
| ATOM | 128S3 | C28 | INH | K | 501 | 44.195 | 3.321 | 6.391 | 1.00 | 78.22 | C | |
| ATOM | 12856 | C31 | INH | K | 501 | 44.099 | 4.248 | 5.162 | 1.00 | 78.11 | C | |
| ATOM | 12859 | C34 | INH | K | 501 | 45.265 | 3.941 | 4.212 | 1.00 | 78.58 | C | |
| ATOM | 12862 | C37 | INH | K | 501 | 45.184 | 2.479 | 3.721 | 1.00 | 78.23 | C | |
| ATOM | 1286S | C5 | INH | K | 501 | 45.115 | 1.515 | 4.893 | 1.00 | 78.13 | C | |
| ATOM | 12866 | N4 | INH | K | 501 | 45.483 | 0.183 | 4.908 | 1.00 | 78.01 | N | |
| ATOM | 12867 | C40 | INH | K | 501 | 45.973 | −0.554 | 3.749 | 1.00 | 78.06 | C | |
| ATOM | 12869 | C48 | INH | K | 501 | 47.444 | −0.321 | 3.349 | 1.00 | 77.48 | C | |
| ATOM | 12872 | C46 | INH | K | 501 | 48.109 | −1.688 | 3.091 | 1.00 | 77.40 | C | |
| ATOM | 12874 | N51 | INH | K | 501 | 49.532 | −1.613 | 2.693 | 1.00 | 76.83 | N | |
| ATOM | 12876 | C53 | INH | K | 501 | 49.991 | −1.553 | 1.286 | 1.00 | 76.02 | C | |
| ATOM | 12880 | C44 | INH | K | 501 | 47.890 | −2.661 | 4.287 | 1.00 | 77.97 | C | |
| ATOM | 12882 | O57 | INH | K | 501 | 48.747 | −2.548 | 5.462 | 1.00 | 78.78 | O | |
| ATOM | 12883 | C58 | INH | K | 501 | 49.004 | −1.304 | 6.139 | 1.00 | 78.56 | C | |
| ATOM | 12887 | C43 | INH | K | 501 | 46.362 | −2.896 | 4.617 | 1.00 | 78.08 | C | |
| ATOM | 12888 | O42 | INH | K | 501 | 45.574 | −1.935 | 3.867 | 1.00 | 78.81 | O | |
| ATOM | 12889 | C62 | INH | K | 501 | 45.792 | −4.164 | 3.906 | 1.00 | 77.58 | C | |
| ATOM | 12893 | N1 | INH | K | 501 | 45.942 | −2.807 | 6.050 | 1.00 | 78.18 | N | |
| ATOM | 12894 | C12 | INH | K | 501 | 45.935 | −3.816 | 7.036 | 1.00 | 78.13 | C | |
| ATOM | 12895 | C11 | INH | K | 501 | 45.458 | −3.313 | 8.208 | 1.00 | 78.12 | C | |
| ATOM | 12896 | C19 | INH | K | 501 | 45.334 | −4.091 | 9.380 | 1.00 | 78.17 | C | |
| ATOM | 12898 | C17 | INH | K | 501 | 45.719 | −5.454 | 9.337 | 1.00 | 78.18 | C | |
| ATOM | 12900 | C15 | INH | K | 501 | 46.213 | −6.008 | 8.130 | 1.00 | 78.05 | C | |
| ATOM | 12902 | C13 | INH | K | 501 | 46.324 | −5.193 | 6.985 | 1.00 | 78.09 | C | |
| ATOM | 12904 | OW0 | HOH | W | 1 | 57.692 | 14.791 | 31.508 | 1.00 | 41.12 | O | |
| ATOM | 12907 | OW0 | HOH | W | 3 | 28.042 | 8.415 | −3.283 | 1.00 | 59.59 | O | |
| ATOM | 12910 | OW0 | NOH | W | 4 | 22.748 | −4.409 | −8.825 | 1.00 | 75.73 | O | |
| ATOM | 12913 | OW0 | HOH | W | 5 | 56.632 | 35.487 | 64.325 | 1.00 | 33.00 | O | |
| ATOM | 12916 | OW0 | HOH | W | 7 | 66.942 | 23.762 | 47.746 | 1.00 | 30.02 | O | |
| ATOM | 12919 | OW0 | HOH | W | 8 | 21.929 | 26.143 | 5.046 | 1.00 | 70.15 | O | |
| ATOM | 12922 | OW0 | HOH | W | 9 | 40.405 | 38.256 | 40.391 | 1.00 | 43.52 | O | |
| ATOM | 12925 | OW0 | HOH | W | 13 | 43.665 | 20.198 | 63.123 | 1.00 | 45.69 | O | |
| ATOM | 12928 | OW0 | HOH | W | 14 | 0.193 | 20.597 | 3.471 | 1.00 | 53.03 | O | |
| ATOM | 12931 | OW0 | HOH | W | 16 | 53.621 | 7.095 | −11.697 | 1.00 | 60.56 | O | |
| ATOM | 12934 | OW0 | HOH | W | 17 | 22.336 | 11.823 | 0.006 | 1.00 | 39.36 | O | |
| ATOM | 12937 | OW0 | HOH | W | 18 | 57.603 | 27.959 | 35.248 | 1.00 | 51.37 | O | |
| ATOM | 12940 | OW0 | HOH | W | 19 | 38.197 | 44.115 | 47.189 | 1.00 | 53.19 | O | |
| ATOM | 12943 | OW0 | HOH | W | 24 | 12.584 | 7.112 | −4.961 | 1.00 | 53.80 | O | |
| ATOM | 12946 | OW0 | HOH | W | 25 | 36.268 | 42.784 | 58.457 | 1.00 | 56.23 | O | |
| ATOM | 12949 | OW0 | HOH | W | 26 | 48.815 | 45.938 | 45.437 | 1.00 | 38.06 | O | |
| ATOM | 12952 | OW0 | HOH | W | 28 | 50.349 | 38.734 | 70.174 | 1.00 | 51.33 | O | |
| ATOM | 12955 | OW0 | HOH | W | 29 | 43.332 | 33.410 | 75.800 | 1.00 | 65.57 | O | |
| ATOM | 12958 | OW0 | HOH | W | 30 | 40.879 | −10.564 | 17.504 | 1.00 | 68.76 | O | |

### EXAMPLE 3

Identification and Design of Specific Inhibitors to c-Abl by Virtual Screening

Using the three dimensional information provided herein, in whole or in part, and conventional techniques of molecular modeling and/or molecular replacement, one can design a specific inhibitor of c-Abl which does not bind to other tyrosine kinases. This may be achieved, e.g., by generation of a model structure for high-throughput docking (HTD).

Generation of the Model Structure for HTD

The program ICM v3.0-25d [see Abagyan and Totrov, Curr Opin Chem Biol, Vol. 5, pp. 375-382 (2001)] is used for creation of the HTD model from a pdb file containing the 2.8 Å resolution structure of human c-Abl kinase co-crystallized with Compound A described in Table 6. The pdb file is read into ICM and all water molecules are deleted. The monomer of a c-Abl kinase (chain B) in the pdb file containing an inhibitor (chain I) bound to the ATP binding site is selected as the model for HTD. The remaining monomers (chains A and C) and inhibitors (chains J and K) are deleted. Hydrogen atoms are added to the remaining c-Abl/inhibitor complex and their coordinates are optimized using ICM. The resulting complex is converted to an ICM-formatted object in preparation of the receptor for HTD. The binding site is defined by selecting all residues of c-Abl kinase with any atoms within 5.0 Å of the inhibitor. The size and location of the box used to define the volume of the electrostatic maps for docking is refined manually in order to focus on the ATP binding site. The electrostatic maps are generated using a grid cell size of 0.5 Å.

Testing the Accuracy of the Receptor Docking Model in ICM

A critical means of assessing the accuracy of HTD algorithms is their ability to reproduce experimentally determined binding poses for compounds which bind to the active site of the receptor. The crystallographically determined coordinates of the ligand, Compound A are extracted from the complex structure used to generate the docking model. Covalent bonds are edited interactively in ICM to generate correct connectivity and bond order. Hydrogen atoms are then added and partial charges are assigned. Docking is carried out against the receptor model through the graphical user interface. The resulting docked structure closely matched the crystallographically determined crystal structure, yielding an RMSD of 0.55 Å. As a result, the receptor model is judged to be sufficiently accurate to proceed with HTD on large databases.

Pre-Processing of the Compound Databases for HTD

The Available Chemicals Directory (ACD) (MDL Information Systems, Inc. Available Chemicals Directory, 2003. San Leandro, Calif.) and a proprietary Novartis Corporate Database (CDB) are screened for compounds with potential inhibitory activity against human c-Abl kinase. These compounds are filtered so that salts are stripped and duplicates are removed. Unity v4.3 (Tripos, Inc. 2003. St. Louis, Mo.) is then used to filter compounds which are either present in mixtures, contain metals, are isotopes, do not possess any carbon atoms, have a ClogP greater than 5.0, or do not possess a molecular weight between 50 Daltons and 500 Daltons. This results in a database containing 140,219 compounds from the ACD and 686,100 compounds from the CDB. The CDB is further processed with Pipeline Pilot v3.0 (Scitegic, Inc. Pipeline Pilot 3.0 2003, San Diego, Calif.) to generate a maximally diverse subset containing 343,050 compounds.

HTD with ICM

All HTD calculations are carried out on a 92 processor Linux cluster consisting of 46 dual-processor Xeon CPUs (2.4 GHz) using the Linux 2.4.20 operating system. The ACD and CDB databases are submitted to docking with ICM against the receptor model of human c-Abl kinase. Partial charges are assigned to the atoms of each compound at the time of docking within ICM. Only the top scoring pose for each compound is retained. The resulting docked poses are written to binary output files and the scores for each compound are written to text files.

Analysis of HTD Results

The top scoring compounds from each database are taken to be those with a score corresponding to three standard deviations below the mean score. This resulted in 76 compounds and 724 compounds selected from the ACD and CDB, respectively. Extended connectivity fingerprints (ECFPs) (Scitegic, Inc. Pipeline Pilot 3.0 2003, San Diego, Calif.) with a neighborhood size of 6 are calculated, as implemented in Pipeline Pilot v3.0. The top scoring compounds from each database are clustered according to their ECFPs in Pipeline Pilot, yielding a total of 4 clusters and 37 clusters for the ACD and CDB, respectively. The top 200 scoring compounds from the combined ACD and CDB datasets are evaluated interactively using molecular graphics, and 48 were selected for further testing.

The top scoring poses are evaluated interactively using molecular graphics to select those compounds which formed favorable intermolecular hydrogen bonds and van der Waals interactions with the c-Abl kinase receptor model, as well as to eliminate compounds containing cis-amides and non-aromatic tautomers. Compounds are also selected for desirable drug-like properties, such as size, lipophilicity and chemical stability. The resulting “hit” compounds are confirmed as leads for drug optimization, by their in vitro evaluation in a test for activity against c-Abl protein tyrosine kinase. The test is conducted as a filter binding assay as follows: The His-tagged kinase domain of c-Abl is cloned and expressed in the baculovirus/Sf9 system. See Bhat et al., J Biol Chem, Vol. 27, pp. 16170-16175 (1997). A protein of 37 kD (c-Abl kinase) is purified by a two-step procedure over a Cobalt metal chelate column followed by an anion exchange column with a yield of 1-2 mg/L of Sf9 cells. The purity of the c-Abl kinase is >90% as judged by SDS-PAGE after Coomassie blue staining. The assay contains: c-Abl kinase (50 ng), 20 mM Tris.HCl, pH 7.5, 10 mM MgCl₂, 10 μM Na₃VO₄, 1 mM DTT and 0.06 μCi/assay [³²P]-ATP (5 μM ATP) using 30 μg/mL poly-Ala,Glu,Lys,Tyr-6:2:5:1 (Poly-AEKY, Sigma P1152) in the presence of 1% DMSO, total volume of 30 μL. Reactions are terminated by adding 10 μL of 250 mM EDTA, and 30 μL of the reaction mixture is transferred onto Immobilon-PVDF membrane (Millipore, Bedford, Mass., USA) previously soaked for 5 minutes with methanol, rinsed with water, then soaked for 5 minutes with 0.5% H₃PO₄ and mounted on vacuum manifold with disconnected vacuum source. After spotting all samples, vacuum is connected and each well-rinsed with 200 μL 0.5% H₃PO₄. Membranes are removed and washed on a shaker with 0.5% H₃PO₄ (4 times) and once with ethanol. Membranes are counted after drying at ambient temperature, mounting in Packard TopCount 96-well frame, and addition of 10 μL/well of Microscint™ (Packard). The activity of the compounds is quantified as IC₅₀ and IC₉₀ values, as determined from their dose response curves by graphical extrapolation. Thus, IC₅₀ values are determined by linear regression analysis of the percentages for the inhibition of each compound in at least four concentrations (as a rule 0.01, 0.1, 1.0 and 10 mol).

Results

Results of virtual screening has identified at least three specific inhibitors of c-Abl kinase. One of these 2-[[2-[3-[4-amino-5-(3-methoxyphenyl)-5H-pyrrolo[3,2-d]pyrimidin-7-yl]phenoxy]ethyl]amino]-ethanol, is disclosed in WO 97/49706 and is known to be a tyrosine kinase inhibitor, thus validating the methods disclosed herein.

1. A crystal of c-Abl comprising unit cell dimensions: a=182.6 Å, b=58.3 Å, c=104.0 Å, beta=119.2°, with space group C2.

2. A three-dimensional structure of c-Abl comprising the atomic structure coordinates of Table 6.

3. A crystal of c-Abl comprising an active conformation of DFG motif.

4. A crystal of claim 3 bound to at least one ligand or low molecular weight compound.

5. A computer readable medium comprising data storage material encoded with computer readable data, wherein said data comprises the atomic coordinates of c-Abl as disclosed in Table 6.

6. A method of determining the three-dimensional structure of c-Abl comprising:
(a) crystallization of a polypeptide of c-Abl, the amino acid sequence of which comprises SEQ ID NO. 4 or a mutant, fragment or homologue thereof; and
(b) utilizing the atomic coordinates of Table 6 in whole or in part to determine the three-dimensional structure of said polypeptide.

7. A method of determining the three-dimensional structure of a complex comprising a polypeptide of c-Abl, the amino acid sequence of which comprises SEQ ID NO. 4, a mutant, fragment or homologue thereof, bound to at least one ligand comprising:
(a) obtaining x-ray diffraction data for crystals of the complex; and
(b) utilizing the atomic coordinates of Table 6 in whole or in part to define the three-dimensional structure of the complex.

8. A method of identifying a ligand or low molecular weight compound that binds to c-Abl comprising:
(a) utilizing the three-dimensional structure of c-Abl derived in whole or in part from the set of atomic coordinates in Table 6 to identify a potential ligand or low molecular weight compound that binds to c-Abl; and
(b) selecting those ligands or low molecular weight compounds that bind to c-Abl.

9. The method of claim 8, wherein the ligand or low molecular weight compound is identified in silico.

10. A method of identifying a ligand or low molecular weight compound that selectively binds to c-Abl comprising:
(a) utilizing the three-dimensional structure of c-Abl derived in whole or in part from the set of atomic coordinates in Table 6 to identify a potential ligand or low molecular weight compound that binds selectively to c-Abl; and
(b) selecting only those ligands or low molecular weight compounds which bind selectively to c-Abl and to no other tyrosine kinase.

11. The method of claim 10, wherein the ligand or low molecular weight compound is identified in silico.

12. A method of designing a ligand or low molecular weight compound capable of binding to c-Abl comprising:
(a) using the atomic coordinates of Table 6 in whole or in part to determine the three dimensional structure of c-Abl;
(b) screening said c-Abl with candidate ligands or low molecular weight compounds to determine which bind to c-Abl;
(c) selecting those ligands or low molecular weight compounds which bind to c-Abl; and
(d) modifying those ligands or low molecular weight compounds which bind to maximize physical properties such as solubility, affinity, specificity or potency.

13. The method of claim 12, wherein the ligand or low molecular weight compound is designed in silico.

14. A method of designing a ligand or low molecular weight compound capable of binding selectively to c-Abl comprising:
(a) using the atomic coordinates of Table 6 in whole or in part to determine the three-dimensional structure of c-Abl;
(b) screening said c-Abl with candidate ligands or low molecular weight compounds to determine which bind selectively to c-Abl and not to any other tyrosine kinase;
(c) selecting those ligands or low molecular weight compounds which bind selectively to c-Abl; and
(d) modifying those ligands or low molecular weight compounds which bind to maximize physical properties, such as solubility, affinity, specificity or potency.

15. The method of claim 14, wherein the ligand or low molecular weight compound is identified in silico.
